(19) United States
(12) Patent Application Publication
(10) Pub. No.: US 2024/0002543 A1
(43) Pub. Date: Jan. 4, 2024

(54) MULTIFUNCTIONAL MOLECULES THAT BIND TO CALRETICULIN AND USES THEREOF

(71) Applicant: Marengo Therapeutics, Inc., Cambridge, MA (US)

(72) Inventors: Andreas Loew, Boston, MA (US); Madan Katragadda, Acton, MA (US); Saeyoung Park, Cambridge, MA (US); Roya Servattalab, Brighton, MA (US)

(21) Appl. No.: 18/173,995

(22) Filed: Feb. 24, 2023

Related U.S. Application Data
(63) Continuation of application No. PCT/US2021/047571, filed on Aug. 25, 2021.
(60) Provisional application No. 63/070,769, filed on Aug. 26, 2020.

(51) Int. Cl. C07K 16/46; A61P 35/00

(52) U.S. Cl. CPC C07K 16/468; A61P 35/00; C07K 2317/52; C07K 2317/565; C07K 2317/31

(57) ABSTRACT

Multifunctional molecules that include i) an antigen binding domain that binds to a calreticulin protein; and one, two or all of: (ii) an immune cell engager (e.g., chosen from an NK cell engager, a T cell engager, a B cell engager, a dendritic cell engager, or a macrophage cell engager); (iii) a cytokine molecule; and/or (iv) a stromal modifying moiety are disclosed. Additionally disclosed are nucleic acids encoding the same, methods of producing the aforesaid molecules, and methods of treating a cancer using the aforesaid molecules.

### RELATED APPLICATIONS

This application is a continuation of International Patent Application No., PCT/US2021/047571 filed Aug. 25, 2021 which claims the benefit of U.S. Provisional Patent Application No. 63/070,769 filed on Aug. 26, 2020, the entire contents of which are hereby incorporated by reference.

### REFERENCE TO A SEQUENCE LISTING XML

The instant application contains a Sequence Listing which has been submitted electronically in XML format and is hereby incorporated by reference in its entirety. Said XML file, created on Feb. 16, 2023, is named 53676-740_301_SL.xml and is 2,710,544 bytes in size.

### BACKGROUND

Myeloproliferative neoplasms (MPNs) are a group of conditions that cause blood cells to grow abnormally in the bone marrow. Common myeloproliferative neoplasms include primary or idiopathic myelofibrosis (MF), essential thrombocytosis (ET), polycythemia vera (PV), and chronic myelogenous leukemia (CML). Primary myelofibrosis is a chronic blood cancer in which excessive scar tissue forms in the bone marrow and impairs its ability to produce normal blood cells. Given the ongoing need for improved treatment of myeloproliferative neoplasms such as myelofibrosis, new compositions and treatments targeting myeloproliferative neoplasms are highly desirable.

### SUMMARY OF THE INVENTION

Provided herein, inter alia, in an aspect, is a composition comprising a polypeptide molecule comprising: (i) a first antigen binding domain that binds to a calreticulin protein (e.g., a wild-type or mutant calreticulin protein), e.g., a calreticulin-targeting antigen binding domain disclosed in any one of Table 4, Table 5, Table 6, Table 24, Table 25, Table 16, Table 17, Table 18, or Table 19, and (ii) a second antigen binding domain that binds to TCRβV, e.g., an anti-TCRβV antigen binding domain disclosed in any one of Table 30, Table 31, Table 32, Table 10, Table 11, Table 12, or Table 13, or a second antigen binding domain that binds to NKp30, e.g., an anti-NKp30 antigen binding domain disclosed in Table 7, Table 8, Table 35, Table 36, Table 9, Table 10, or Table 34.

In some embodiments, the polypeptide molecule is a multifunctional polypeptide molecule.

In some embodiments, the polypeptide molecule is a multispecific polypeptide molecule.

In some embodiments, the second antigen binding domain binds to TCRβV.

In some embodiments, the second antigen binding domain activates a T cell or the second antigen binding domain does not activate a T cell.

In some embodiments, the second antigen binding domain binds to TCRβV12 or TCRβV6 (e.g., comprising the amino acid sequence of SEQ ID NO: 1044).

In some embodiments, the second antigen binding domain comprises one or more amino acid sequences as listed in Table 30, Table 31, Table 32, Table 10, Table 11, Table 12, or Table 13.

In some embodiments, the second antigen binding domain comprises: (a) a heavy chain variable region (VH) and/or a light chain variable region (VL), wherein: (i) the VH comprises a heavy chain complementarity determining region 1 (VHCDR1) having an amino acid sequence of a VHCDR1 in Table 30, Table 31, Table 10, Table 11, Table 12, or Table 13 (or a sequence with no more than 1, 2, 3, or 4 mutations, e.g., substitutions, additions, or deletions), a VHCDR2 having an amino acid sequence of a VHCDR2 in Table 30, Table 31, Table 10, Table 11, Table 12, or Table 13 (or a sequence with no more than 1, 2, 3, or 4 mutations, e.g., substitutions, additions, or deletions), and/or a VHCDR3 having an amino acid sequence of a VHCDR3 in Table 30, Table 31, Table 10, Table 11, Table 12, or Table 13 (or a sequence with no more than 1, 2, 3, or 4 mutations, e.g., substitutions, additions, or deletions), (ii) the VL comprises a light chain complementarity determining region 1 (VLCDR1) having an amino acid sequence of a VLCDR1 in Table 30, Table 31, Table 10, Table 11, Table 12, or Table 13 (or a sequence with no more than 1, 2, 3, or 4 mutations, e.g., substitutions, additions, or deletions), a VLCDR2 having an amino acid sequence of a VLCDR2 in Table 30, Table 31, Table 10, Table 11, Table 12, or Table 13 (or a sequence with no more than 1, 2, 3, or 4 mutations, e.g., substitutions, additions, or deletions), and/or a VLCDR3 having an amino acid sequence of a VLCDR3 in Table 30, Table 31, Table 10, Table 11, Table 12, or Table 13 (or a sequence with no more than 1, 2, 3, or 4 mutations, e.g., substitutions, additions, or deletions); (b) a heavy chain variable region (VH) and/or a light chain variable region (VL), wherein: (i) the VH comprises a heavy chain complementarity determining region 1 (VHCDR1) amino acid sequence of SEQ ID NO: 3 (or a sequence with no more than 1, 2, 3, or 4 mutations, e.g., substitutions, additions, or deletions), a VHCDR2 amino acid sequence of SEQ ID NO: 4 (or a sequence with no more than 1, 2, 3, or 4 mutations, e.g., substitutions, additions, or deletions), and/or a VHCDR3 amino acid sequence of SEQ ID NO: 5 (or a sequence with no more than 1, 2, 3, or 4 mutations, e.g., substitutions, additions, or deletions), and/or (ii) the VL comprises a light chain complementarity determining region 1 (VLCDR1) amino acid sequence of SEQ ID NO: 6 (or a sequence with no more than 1, 2, 3, or 4 mutations, e.g., substitutions, additions, or deletions), a VLCDR2 amino acid sequence of SEQ ID NO: 7 (or a sequence with no more than 1, 2, 3, or 4 mutations, e.g., substitutions, additions, or deletions), and/or a VLCDR3 amino acid sequence of SEQ ID NO: 8 (or a sequence with no more than 1, 2, 3, or 4 mutations, e.g., substitutions, additions, or deletions); (c) a heavy chain variable region (VH) and/or a light chain variable region (VL), wherein: (i) the VH comprises a heavy chain complementarity determining region 1 (VHCDR1) amino acid sequence of SEQ ID NO: 45 (or a sequence with no more than 1, 2, 3, or 4 mutations, e.g., substitutions, additions, or deletions), a VHCDR2 amino acid sequence of SEQ ID NO: 46 (or a sequence with no more than 1, 2, 3, or 4 mutations, e.g., substitutions, additions, or deletions), and/or a VHCDR3 amino acid sequence of SEQ ID NO: 47 (or a sequence with no more than 1, 2, 3, or 4 mutations, e.g., substitutions, additions, or deletions), and/or (ii) the VL comprises a light chain complementarity determining region 1 (VLCDR1) amino acid sequence of SEQ ID NO: 51 (or a sequence with no more than 1, 2, 3, or 4 mutations, e.g., substitutions, additions, or deletions), a VLCDR2 amino acid sequence of SEQ ID NO: 52 (or a sequence with no more than 1, 2, 3, or 4 mutations, e.g., substitutions, additions, or deletions), and/or a VLCDR3 amino acid sequence of SEQ ID NO: 53 (or a sequence with no more than 1, 2, 3, or 4 mutations, e.g., substitutions, additions, or deletions); and/or (d) a heavy chain variable region (VH) and/or a light chain variable region (VL), wherein: (i) the VH comprises a heavy chain complementarity determining region 1 (VHCDR1) amino acid sequence of SEQ ID NO: 48 (or a sequence with no more than 1, 2, 3, or 4 mutations, e.g., substitutions, additions, or deletions), a VHCDR2 amino acid sequence of SEQ ID NO: 49 (or a sequence with no more than 1, 2, 3, or 4 mutations, e.g., substitutions, additions, or deletions), and/or a VHCDR3 amino acid sequence of SEQ ID NO: 50 (or a sequence with no more than 1, 2, 3, or 4 mutations, e.g., substitutions, additions, or deletions), and/or (ii) the VL comprises a light chain complementarity determining region 1 (VLCDR1) amino acid sequence of SEQ ID NO: 54 (or a sequence with no more than 1, 2, 3, or 4 mutations, e.g., substitutions, additions, or deletions), a VLCDR2 amino acid sequence of SEQ ID NO: 55 (or a sequence with no more than 1, 2, 3, or 4 mutations, e.g., substitutions, additions, or deletions), and/or a VLCDR3 amino acid sequence of SEQ ID NO: 56 (or a sequence with no more than 1, 2, 3, or 4 mutations, e.g., substitutions, additions, or deletions).

In some embodiments, the second antigen binding domain comprises: (a) a heavy chain variable region (VH) and/or a light chain variable region (VL), wherein: (i) the VH comprises the amino acid sequence of a VH in Table 30, Table 31, Table 10, Table 11, Table 12, or Table 13 (or an amino acid sequence having at least about 77%, 80%, 85%, 90%, 95%, or 99% sequence identity thereto), and/or (ii) the VL comprises the amino acid sequence of a VL in Table 30, Table 31, Table 10, Table 11, Table 12, or Table 13 (or an amino acid sequence having at least about 77%, 80%, 85%, 90%, 95%, or 99% sequence identity thereto) (iii) the VH comprises the amino acid sequence of SEQ ID NO: 9 (or an amino acid sequence having at least about 77%, 80%, 85%, 90%, 95%, or 99% sequence identity thereto), and/or (iv) the VL comprises the amino acid sequence of SEQ ID NO: 10 (or an amino acid sequence having at least about 77%, 80%, 85%, 90%, 95%, or 99% sequence identity thereto); (b) a heavy chain variable region (VH) and/or a light chain variable region (VL), wherein: (i) the VH comprises the amino acid sequence of SEQ ID NO: 9 (or an amino acid sequence having at least about 77%, 80%, 85%, 90%, 95%, or 99% sequence identity thereto), and/or (ii) the VL comprises the amino acid sequence of SEQ ID NO: 11 (or an amino acid sequence having at least about 77%, 80%, 85%, 90%, 95%, or 99% sequence identity thereto); and/or (c) a heavy chain variable region (VH) and/or a light chain variable region (VL), wherein: (i) the VH comprises the amino acid sequence of SEQ ID NO: 1312 (or an amino acid sequence having at least about 77%, 80%, 85%, 90%, 95%, or 99% sequence identity thereto), and/or (ii) the VL comprises the amino acid sequence of SEQ ID NO: 1314 (or an amino acid sequence having at least about 77%, 80%, 85%, 90%, 95%, or 99% sequence identity thereto).

In some embodiments, the second antigen binding domain comprises: (a) a heavy chain variable region (VH) and/or a light chain variable region (VL), wherein: (i) the VH comprises a heavy chain complementarity determining region 1 (VHCDR1) amino acid sequence of SEQ ID NO: 17 (or a sequence with no more than 1, 2, 3, or 4 mutations, e.g., substitutions, additions, or deletions), a VHCDR2 amino acid sequence of SEQ ID NO: 18 (or a sequence with no more than 1, 2, 3, or 4 mutations, e.g., substitutions, additions, or deletions), and/or a VHCDR3 amino acid sequence of SEQ ID NO: 19 (or a sequence with no more than 1, 2, 3, or 4 mutations, e.g., substitutions, additions, or deletions), and/or (ii) the VL comprises a light chain complementarity determining region 1 (VLCDR1) amino acid sequence of SEQ ID NO: 20 (or a sequence with no more than 1, 2, 3, or 4 mutations, e.g., substitutions, additions, or deletions), a VLCDR2 amino acid sequence of SEQ ID NO: 21 (or a sequence with no more than 1, 2, 3, or 4 mutations, e.g., substitutions, additions, or deletions), and/or a VLCDR3 amino acid sequence of SEQ ID NO: 22 (or a sequence with no more than 1, 2, 3, or 4 mutations, e.g., substitutions, additions, or deletions); (b) a heavy chain variable region (VH) and/or a light chain variable region (VL), wherein: (i) the VH comprises a heavy chain complementarity determining region 1 (VHCDR1) amino acid sequence of SEQ ID NO: 57 (or a sequence with no more than 1, 2, 3, or 4 mutations, e.g., substitutions, additions, or deletions), a VHCDR2 amino acid sequence of SEQ ID NO: 58 (or a sequence with no more than 1, 2, 3, or 4 mutations, e.g., substitutions, additions, or deletions), and/or a VHCDR3 amino acid sequence of SEQ ID NO: 59 (or a sequence with no more than 1, 2, 3, or 4 mutations, e.g., substitutions, additions, or deletions), and/or (ii) the VL comprises a light chain complementarity determining region 1 (VLCDR1) amino acid sequence of SEQ ID NO: 63 (or a sequence with no more than 1, 2, 3, or 4 mutations, e.g., substitutions, additions, or deletions), a VLCDR2 amino acid sequence of SEQ ID NO: 64 (or a sequence with no more than 1, 2, 3, or 4 mutations, e.g., substitutions, additions, or deletions), and/or a VLCDR3 amino acid sequence of SEQ ID NO: 65 (or a sequence with no more than 1, 2, 3, or 4 mutations, e.g., substitutions, additions, or deletions); and/or (c) a heavy chain variable region (VH) and/or a light chain variable region (VL), wherein: (i) the VH comprises a heavy chain complementarity determining region 1 (VHCDR1) amino acid sequence of SEQ ID NO: 60 (or a sequence with no more than 1, 2, 3, or 4 mutations, e.g., substitutions, additions, or deletions), a VHCDR2 amino acid sequence of SEQ ID NO: 61 (or a sequence with no more than 1, 2, 3, or 4 mutations, e.g., substitutions, additions, or deletions), and/or a VHCDR3 amino acid sequence of SEQ ID NO: 62 (or a sequence with no more than 1, 2, 3, or 4 mutations, e.g., substitutions, additions, or deletions), and/or (ii) the VL comprises a light chain complementarity determining region 1 (VLCDR1) amino acid sequence of SEQ ID NO: 66 (or a sequence with no more than 1, 2, 3, or 4 mutations, e.g., substitutions, additions, or deletions), a VLCDR2 amino acid sequence of SEQ ID NO: 67 (or a sequence with no more than 1, 2, 3, or 4 mutations, e.g., substitutions, additions, or deletions), and/or a VLCDR3 amino acid sequence of SEQ ID NO: 68 (or a sequence with no more than 1, 2, 3, or 4 mutations, e.g., substitutions, additions, or deletions).

In some embodiments, the second antigen binding domain comprises: (a) a heavy chain variable region (VH) and/or a light chain variable region (VL), wherein: (i) the VH comprises the amino acid sequence of SEQ ID NO: 15 (or an amino acid sequence having at least about 77%, 80%, 85%, 90%, 95%, or 99% sequence identity thereto), and/or (ii) the VL comprises the amino acid sequence of SEQ ID NO: 16 (or an amino acid sequence having at least about 77%, 80%, 85%, 90%, 95%, or 99% sequence identity thereto); and/or (b) a heavy chain variable region (VH) and/or a light chain variable region (VL), wherein: (i) the VH comprises: the amino acid sequence of SEQ ID NO: 23 (or an amino acid sequence having at least about 77%, 80%, 85%, 90%, 95%, or 99% sequence identity thereto), the amino acid sequence of SEQ ID NO: 24 (or an amino acid sequence having at least about 77%, 80%, 85%, 90%, 95%, or 99% sequence identity thereto), or the amino acid sequence of SEQ ID NO: 25 (or an amino acid sequence having at least about 77%, 80%, 85%, 90%, 95%, or 99% sequence identity thereto); and/or (ii) the VL comprises: the amino acid sequence of SEQ ID NO: 26 (or an amino acid sequence having at least about 77%, 80%, 85%, 90%, 95%, or 99% sequence identity thereto), the amino acid sequence of SEQ ID NO: 27 (or an amino acid sequence having at least about 77%, 80%, 85%, 90%, 95%, or 99% sequence identity thereto), the amino acid sequence of SEQ ID NO: 28 (or an amino acid sequence having at least about 77%, 80%, 85%, 90%, 95%, or 99% sequence identity thereto), the amino acid sequence of SEQ ID NO: 29 (or an amino acid sequence having at least about 77%, 80%, 85%, 90%, 95%, or 99% sequence identity thereto), or the amino acid sequence of SEQ ID NO: 30 (or an amino acid sequence having at least about 77%, 80%, 85%, 90%, 95%, or 99% sequence identity thereto).

In some embodiments, the composition as describe herein comprises: a first polypeptide comprising, e.g., from N-terminus to C-terminus, a first VL and a first CL, a second polypeptide comprising, e.g., from N-terminus to C-terminus, a first VH, a first CH1, a first dimerization domain (e.g., a first Fc), and a first moiety that binds to TCR (e.g., TCRVβ) (e.g., a first scFv that binds to TCR (e.g., TCRVβ), a third polypeptide comprising, e.g., from N-terminus to C-terminus, a second VH, a second CH1, a second dimerization domain (e.g., a second Fc), and optionally a second moiety that binds to TCR (e.g., TCRVβ) (e.g., a second scFv that binds to TCR (e.g., TCRVβ), a fourth polypeptide comprising, e.g., from N-terminus to C-terminus, a second VL and a second CL, wherein: the first VL and the first VH form a first antigen binding domain that binds to a first calreticulin protein, and the second VL and the second VH form a third antigen binding domain that binds to a second calreticulin protein, optionally wherein the first and second calreticulin proteins comprise the amino acid sequence of SEQ ID NO: 6285, 1001, or 6286, optionally wherein the first and second calreticulin mutant proteins are each independently chosen from: a molecule comprising the amino acid sequence of SEQ ID NO: 6313, or a molecule comprising the amino acid sequence of SEQ ID NO: 6314, optionally wherein the multifunctional molecule comprises the configuration of FIG. 3A or 3B.

In some embodiments, the second antigen binding domain binds to NKp30.

In some embodiments, the second antigen binding domain is chosen from an antibody molecule, e.g., an antigen binding domain, or ligand that binds to (e.g., activates) NKp30, e.g., the second antigen binding domain is an antibody molecule or ligand that binds to (e.g., activates) NKp30.

In some embodiments, the second antigen binding domain comprises: (i) a heavy chain variable region (VH) comprising a heavy chain complementarity determining region 1 (VHCDR1) having an amino acid sequence of a VHCDR1 of Table 7, Table 35, Table 9, Table 10, or Table 34 (or a sequence with no more than 1, 2, 3, or 4 mutations, e.g., substitutions, additions, or deletions), a VHCDR2 having an amino acid sequence of a VHCDR2 of Table 7, Table 35, Table 9, Table 10, or Table 34 (or a sequence with no more than 1, 2, 3, or 4 mutations, e.g., substitutions, additions, or deletions), and/or a VHCDR3 having an amino acid sequence of a VHCDR3 of Table 7, Table 35, Table 9, Table 10, or Table 34 (or a sequence with no more than 1, 2, 3, or 4 mutations, e.g., substitutions, additions, or deletions), and/or (ii) a light chain variable region (VL) comprising a light chain complementarity determining region 1 (VLCDR1) having an amino acid sequence of a VLCDR1 of Table 8, Table 36, Table 9, Table 10, or Table 34 (or a sequence with no more than 1, 2, 3, or 4 mutations, e.g., substitutions, additions, or deletions), a VLCDR2 having an amino acid sequence of a VLCDR2 of Table 8, Table 36, Table 9, Table 10, or Table 34 (or a sequence with no more than 1, 2, 3, or 4 mutations, e.g., substitutions, additions, or deletions), and/or a VLCDR3 having an amino acid sequence of a VLCDR3 of Table 8, Table 36, Table 9, Table 10, or Table 34 (or a sequence with no more than 1, 2, 3, or 4 mutations, e.g., substitutions, additions, or deletions).

In some embodiments, the second antigen binding domain comprises: (i) a heavy chain variable region (VH) comprising a heavy chain complementarity determining region 1 (VHCDR1) amino acid sequence of SEQ ID NO: 7313 (or a sequence with no more than 1, 2, 3, or 4 mutations, e.g., substitutions, additions, or deletions), a VHCDR2 amino acid sequence of SEQ ID NO: 6001 (or a sequence with no more than 1, 2, 3, or 4 mutations, e.g., substitutions, additions, or deletions, and/or a VHCDR3 amino acid sequence of SEQ ID NO: 7315 (or a sequence with no more than 1, 2, 3, or 4 mutations, e.g., substitutions, additions, or deletions; and/or (ii) a light chain variable region (VL) comprising a light chain complementarity determining region 1 (VLCDR1) amino acid sequence of SEQ ID NO: 7326 (or a sequence with no more than 1, 2, 3, or 4 mutations, e.g., substitutions, additions, or deletions), a VLCDR2 amino acid sequence of SEQ ID NO: 7327 (or a sequence with no more than 1, 2, 3, or 4 mutations, e.g., substitutions, additions, or deletions), and/or a VLCDR3 amino acid sequence of SEQ ID NO: 7329 (or a sequence with no more than 1, 2, 3, or 4 mutations, e.g., substitutions, additions, or deletions).

In some embodiments, the second antigen binding domain comprises: (i) a VH comprising the amino acid sequence of any of SEQ ID NOs: 7298 or 7300-7304 (or an amino acid sequence having at least about 75%, 80%, 85%, 90%, 95%, or 99% sequence identity to any of SEQ ID NOs: 7298 or 7300-7304); and/or (ii) a VL comprising the amino acid sequence of any of SEQ ID NOs: 7299 or 7305-7309 (or an amino acid sequence having at least about 93%, 95%, or 99% sequence identity to any of SEQ ID NOs: 7299 or 7305-7309).

In some embodiments, the second antigen binding domain comprises: (i) a VH comprising the amino acid sequence of SEQ ID NO: 7302 (or an amino acid sequence having at least about 75%, 80%, 85%, 90%, 95%, or 99% sequence identity to 7302), and a VL comprising the amino acid sequence of SEQ ID NO: 7305 (or an amino acid sequence having at least about 75%, 80%, 85%, 90%, 95%, or 99% sequence identity to 7305); or (ii) a VH comprising the amino acid sequence of SEQ ID NO: 7302 (or an amino acid sequence having at least about 75%, 80%, 85%, 90%, 95%, or 99% sequence identity to 7302), and a VL comprising the amino acid sequence of SEQ ID NO: 7309 (or an amino acid sequence having at least about 75%, 80%, 85%, 90%, 95%, or 99% sequence identity to 7309).

In some embodiments, the second antigen binding domain comprises: (i) an amino acid sequence of SEQ ID NO: 7310 (or an amino acid sequence having at least about 75%, 80%, 85%, 90%, 95%, or 99% sequence identity to 7310); or (ii) an amino acid sequence of SEQ ID NO: 7311 (or an amino acid sequence having at least about 75%, 80%, 85%, 90%, 95%, or 99% sequence identity to 7311).

In some embodiments, the second antigen binding domain comprises: a heavy chain variable region (VH) comprising a heavy chain complementarity determining region 1 (VHCDR1) amino acid sequence of SEQ ID NO: 6000, a VHCDR2 amino acid sequence of SEQ ID NO: 6001, and/or a VHCDR3 amino acid sequence of SEQ ID NO: 6002, and (ii) a light chain variable region (VL) comprising a light chain complementarity determining region 1 (VLCDR1) amino acid sequence of SEQ ID NO: 6063, a VLCDR2 amino acid sequence of SEQ ID NO: 6064, and/or a VLCDR3 amino acid sequence of SEQ ID NO: 7293.

In some embodiments, the second antigen binding domain comprises: (1) a heavy chain variable region (VH) comprising a heavy chain framework region 1 (VHFWR1) having an amino acid sequence of a VHFWR1 of Table 7, Table 35, Table 9, Table 10, or Table 34 (or a sequence with no more than 1, 2, 3, 4, 5, or 6 mutations, e.g., substitutions, additions, or deletions, therefrom), a VHFWR2 having an amino acid sequence of a VHFWR2 of Table 7, Table 35, Table 9, Table 10, or Table 34 (or a sequence with no more than 1, 2, 3, 4, 5, or 6 mutations, e.g., substitutions, additions, or deletions, therefrom), a VHFWR3 having an amino acid sequence of a VHFWR3 of Table 7, Table 35, Table 9, Table 10, or Table 34 (or a sequence with no more than 1, 2, 3, 4, 5, or 6 mutations, e.g., substitutions, additions, or deletions, therefrom), or a VHFWR4 having an amino acid sequence of a VHFWR4 of Table 7, Table 35, Table 9, Table 10, or Table 34 (or a sequence with no more than 1, 2, 3, 4, 5, or 6 mutations, e.g., substitutions, additions, or deletions, therefrom), and/or (2) a light chain variable region (VL) comprising a light chain framework region 1 (VLFWR1) having an amino acid sequence of a VLFWR1 of Table 8, Table 36, Table 9, Table 10, or Table 34 (or a sequence with no more than 1, 2, 3, 4, 5, or 6 mutations, e.g., substitutions, additions, or deletions, therefrom), a VLFWR2 having an amino acid sequence of a VLFWR2 of Table 8, Table 36, Table 9, Table 10, or Table 34 (or a sequence with no more than 1, 2, 3, 4, 5, or 6 mutations, e.g., substitutions, additions, or deletions, therefrom), a VLFWR3 having an amino acid sequence of a VLFWR3 of Table 8, Table 36, Table 9, Table 10, or Table 34 (or a sequence with no more than 1, 2, 3, 4, 5, or 6 mutations, e.g., substitutions, additions, or deletions, therefrom), or a VLFWR4 having an amino acid sequence of a VLFWR4 of Table 8, Table 36, Table 9, Table 10, or Table 34 (or a sequence with no more than 1, 2, 3, 4, 5, or 6 mutations, e.g., substitutions, additions, or deletions, therefrom).

In some embodiments, the second antigen binding domain comprises: (1) a heavy chain variable region (VH) comprising a heavy chain framework region 1 (VHFWR1) amino acid sequence of SEQ ID NO: 6003, a VHFWR2 amino acid sequence of SEQ ID NO: 6004, a VHFWR3 amino acid sequence of SEQ ID NO: 6005, or a VHFWR4 amino acid sequence of SEQ ID NO: 6006, and (3) a light chain variable region (VL) comprising a light chain framework region 1 (VLFWR1) amino acid sequence of SEQ ID NO: 6066, a VLFWR2 amino acid sequence of SEQ ID NO: 6067, a VLFWR3 amino acid sequence of SEQ ID NO: 7292, or a VLFWR4 amino acid sequence of SEQ ID NO: 6069.

In some embodiments, the second antigen binding domain comprises: (i) a VH comprising the amino acid sequence of a VH of Table 7, Table 35, Table 9, Table 10, or Table 34 (or an amino acid sequence having at least about 75%, 80%, 85%, 90%, 95%, or 99% sequence identity thereto), and/or (ii) a VL comprising the amino acid sequence of a VL of Table 8, Table 36, Table 9, Table 10, or Table 34 (or an amino acid sequence having at least about 93%, 95%, or 99% sequence identity thereto).

In some embodiments, the second antigen binding domain comprises a heavy chain comprising the amino acid sequence of a heavy chain of Table 10 (or an amino acid sequence having at least about 75%, 80%, 85%, 90%, 95%, or 99% sequence identity thereto).

In some embodiments, the second antigen binding domain comprises a light chain comprising the amino acid sequence of a light chain of Table 10 (or an amino acid sequence having at least about 75%, 80%, 85%, 90%, 95%, or 99% sequence identity thereto).

In some embodiments, the second antigen binding domain comprises a heavy chain comprising the amino acid sequence of a heavy chain of Table 10 (or an amino acid sequence having at least about 75%, 80%, 85%, 90%, 95%, or 99% sequence identity thereto), and a light chain comprising the amino acid sequence of a light chain of Table 10 (or an amino acid sequence having at least about 75%, 80%, 85%, 90%, 95%, or 99% sequence identity thereto).

In some embodiments, the composition as described herein comprises: a first polypeptide comprising, e.g., from N-terminus to C-terminus, a first VL and a first CL, a second polypeptide comprising, e.g., from N-terminus to C-terminus, a first VH, a first CH1, a first dimerization domain (e.g., a first Fc), and a first moiety that binds to NKp30 (e.g., a first antibody molecule or ligand that binds to NKp30), a third polypeptide comprising, e.g., from N-terminus to C-terminus, a second VH, a second CH1, a second dimerization domain (e.g., a second Fc), and optionally a second moiety that binds to NKp30 (e.g., a second antibody molecule or ligand that binds to NKp30), a fourth polypeptide comprising, e.g., from N-terminus to C-terminus, a second VL and a second CL, wherein: the first VL and the first VH form a first antigen binding domain that binds to a first calreticulin protein, and the second VL and the second VH from a third antigen binding domain that binds to a second calreticulin protein, optionally wherein the first and second calreticulin proteins comprise the amino acid sequence of SEQ ID NO: 6285, 1001, or 6286, optionally wherein the first and second calreticulin mutant proteins are each independently chosen from: a molecule comprising the amino acid sequence of SEQ ID NO: 6313, or a molecule comprising the amino acid sequence of SEQ ID NO: 6314, optionally wherein the multifunctional molecule comprises the configuration of FIG. 3A or 3B.

In some embodiments, the calreticulin protein comprises an amino acid sequence chosen from SEQ ID NOs: 6285-6312 or 1001, optionally wherein the calreticulin protein comprises an amino acid sequence chosen from SEQ ID NOs: 6313-6346 or 1002-1003.

In some embodiments, the calreticulin protein comprises the amino acid sequence of SEQ ID NO: 6285 or 1001.

In some embodiments, the calreticulin protein comprises the amino acid sequence of SEQ ID NO: 6286.

In some embodiments, the first antigen binding domain binds to an epitope located within the C-terminus of the calreticulin protein, optionally wherein the first antigen binding domain binds to an epitope located within the amino acid sequence of SEQ ID NO: 6285, 1001, or 6286.

In some embodiments, the composition as described herein further comprises a third antigen binding domain that binds to a second calreticulin protein, e.g., wherein the second calreticulin mutant protein comprises the amino acid sequence of SEQ ID NO: 6285, 1001, or 6286, optionally wherein: (i) the third antigen binding domain is different from the first antigen binding domain, or (ii) the third antigen binding domain is the same as the first antigen binding domain.

In some embodiments, the second calreticulin molecule is the same as the calreticulin molecule bound by the first antigen binding domain.

In some embodiments, the second calreticulin molecule is different from the calreticulin molecule bound by the first antigen binding domain.

In some embodiments, the second calreticulin protein comprises an amino acid sequence chosen from SEQ ID NOs: 6285-6312 or 1001, optionally wherein the second calreticulin protein comprises an amino acid sequence chosen from SEQ ID NOs: 6313-6346 or 1002-1003.

In some embodiments, the calreticulin protein bound by the first antigen binding domain comprises the amino acid sequence of SEQ ID NO: 6285 or 1001, and the second calreticulin protein comprises the amino acid sequence of SEQ ID NO: 6286.

In some embodiments, the third antigen binding domain binds to an epitope located within the C-terminus of the second calreticulin protein, optionally wherein the third antigen binding domain binds to an epitope located within the amino acid sequence of SEQ ID NO: 6285, 1001, or 6286.

In some embodiments, the first antigen binding domain comprises: (i) a heavy chain variable region (VH) comprising a heavy chain complementarity determining region 1 (VHCDR1) having an amino acid sequence of a VHCDR1 in Table 4, Table 24, Table 25, or Table 17 (or a sequence with no more than 1, 2, 3, or 4 mutations, e.g., substitutions, additions, or deletions), a VHCDR2 having an amino acid sequence of a VHCDR2 in Table 4, Table 24, Table 25, or Table 17 (or a sequence with no more than 1, 2, 3, or 4 mutations, e.g., substitutions, additions, or deletions), and/or a VHCDR3 having an amino acid sequence of a VHCDR3 in Table 4, Table 24, Table 25, or Table 17 (or a sequence with no more than 1, 2, 3, or 4 mutations, e.g., substitutions, additions, or deletions); (ii) a light chain variable region (VL) comprising a light chain complementarity determining region 1 (VLCDR1) having an amino acid sequence of a VLCDR1 in Table 5, Table 24, Table 25, or Table 18 (or a sequence with no more than 1, 2, 3, or 4 mutations, e.g., substitutions, additions, or deletions), a VLCDR2 having an amino acid sequence of a VLCDR2 in Table 5, Table 24, Table 25, or Table 18 (or a sequence with no more than 1, 2, 3, or 4 mutations, e.g., substitutions, additions, or deletions), and/or a VLCDR3 having an amino acid sequence of a VLCDR3 in Table 5, Table 24, Table 25, or Table 18 (or a sequence with no more than 1, 2, 3, or 4 mutations, e.g., substitutions, additions, or deletions); (iii) a VH comprising the amino acid sequence of a VH in Table 24, Table 25, or Table 16 (or an amino acid sequence having at least about 77%, 80%, 85%, 90%, 95%, or 99% sequence identity thereto); (iv) a VL comprising the amino acid sequence of a VL in Table 24, Table 25, or Table 16 (or an amino acid sequence having at least about 93%, 95%, or 99% sequence identity thereto); (v) a VH comprising a heavy chain framework region 1 (VHFWR1) having an amino acid sequence of a VHFWR1 in Table 4 or Table 6 (or a sequence with no more than 1, 2, 3, 4, 5, 6, 7, 8, or 9 mutations, e.g., substitutions, additions, or deletions), a VHFWR2 having an amino acid sequence of a VHFWR2 in Table 4 or Table 6 (or a sequence with no more than 1, 2, 3, 4, 5, 6, 7, 8, or 9 mutations, e.g., substitutions, additions, or deletions), a VHFWR3 having an amino acid sequence of a VHFWR3 in Table 4 or Table 6 (or a sequence with no more than 1, 2, 3, 4, 5, 6, 7, 8, or 9 mutations, e.g., substitutions, additions, or deletions), and/or a VHFWR4 having an amino acid sequence of a VHFWR4 in Table 4 or Table 6 (or a sequence with no more than 1, 2, 3, 4, 5, 6, 7, 8, or 9 mutations, e.g., substitutions, additions, or deletions), and/or (vi) a VL comprising a light chain framework region 1 (VLFWR1) having an amino acid sequence of a VLFWR1 in Table 5 or Table 6 (or a sequence with no more than 1, 2, 3, 4, 5, 6, 7, 8, or 9 mutations, e.g., substitutions, additions, or deletions), a VLFWR2 having an amino acid sequence of a VLFWR2 in Table 5 or Table 6 (or a sequence with no more than 1, 2, 3, 4, 5, 6, 7, 8, or 9 mutations, e.g., substitutions, additions, or deletions), a VLFWR3 having an amino acid sequence of a VLFWR3 in Table 5 or Table 6 (or a sequence with no more than 1, 2, 3, 4, 5, 6, 7, 8, or 9 mutations, e.g., substitutions, additions, or deletions), and/or a VLFWR4 having an amino acid sequence of a VLFWR4 in Table 5 or Table 6 (or a sequence with no more than 1, 2, 3, 4, 5, 6, 7, 8, or 9 mutations, e.g., substitutions, additions, or deletions).

In some embodiments, the multifunctional molecule further comprises a tumor-targeting moiety.

In some embodiments, the tumor-targeting moiety binds to a tumor antigen.

In some embodiments, the tumor antigen is selected from G6B, CD34, CD41, P-selectin, Clec2, cKIT, FLT3, MPL, ITGB3, ITGB2, GP5, GP6, GP9, GP1BA, DSC2, FCGR2A, TNFRSF10A, TNFRSF10B, or TM4SF1.

In some embodiments, the tumor-targeting moiety comprises an antibody molecule, e.g., that binds to a tumor antigen selected from G6B, CD34, CD41, P-selectin, Clec2, cKIT, FLT3, MPL, ITGB3, ITGB2, GP5, GP6, GP9, GP1BA, DSC2, FCGR2A, TNFRSF10A, TNFRSF10B, or TM4SF1.

In some embodiments, the tumor-targeting moiety comprises a VH and/or VL sequence, e.g., as listed in Table 38 or Table 20.

In some embodiments, the multifunctional molecule preferentially binds to a myeloproliferative neoplasm cell over a non-tumor cell, optionally wherein the binding between the multifunctional molecule and the myeloproliferative neoplasm cell is more than 10, 20, 30, 40, 50-fold greater than the binding between the multifunctional molecule and a non-tumor cell.

In some embodiments, the myeloproliferative neoplasm cell is chosen from a myelofibrosis cell, an essential thrombocythemia cell, a polycythemia vera cell, or a chronic myeloid cancer cell, optionally wherein: the myeloproliferative neoplasm cell does not comprise a JAK2 V617F mutation, or the myeloproliferative neoplasm cell does not comprise a MPL mutation.

In some embodiments, the composition as described herein further comprises a linker, e.g., a linker between the first antigen binding domain and the second antigen binding domain.

In some embodiments, the linker is chosen from: a cleavable linker, a non-cleavable linker, a peptide linker, a flexible linker, a rigid linker, a helical linker, or a non-helical linker.

In some embodiments, the linker is a peptide linker.

In some embodiments, the peptide linker comprises Gly and Ser.

In some embodiments, the peptide linker comprises an amino acid sequence chosen from SEQ ID NOs: 6214-6217 or 6220-6221 and 77-78.

In another aspect, provides herein is a multifunctional molecule comprising: (i) a first antigen binding domain that binds to a calreticulin protein (e.g., a wild-type or mutant calreticulin protein), e.g., a calreticulin-targeting antigen binding domain disclosed in any one of Table 4, Table 5, Table 6, Table 24, Table 25, Table 16, Table 17, Table 18, or Table 19, and (ii) a second antigen binding domain that binds to TCRβV, e.g., an anti-TCRβV antigen binding domain disclosed in any one of Table 30, Table 31, Table 32, Table 10, Table 11, Table 12, or Table 13, or a second antigen binding domain that binds to NKp30, e.g., an anti-NKp30 antigen binding domain disclosed in Table 7, Table 8, Table 35, Table 36, Table 9, Table 10, or Table 34.

In some embodiments, the second antigen binding domain binds to TCRβV.

In some embodiments, the second antigen binding domain activates a T cell or the second antigen binding domain does not activate a T cell.

In some embodiments, the second antigen binding domain binds to TCRβV12 or TCRβV6 (e.g., comprising the amino acid sequence of SEQ ID NO: 1044).

In some embodiments, the second antigen binding domain comprises one or more amino acid sequences as listed in Table 30, Table 31, Table 32, Table 10, Table 11, Table 12, or Table 13.

In some embodiments, the second antigen binding domain comprises: (a) a heavy chain variable region (VH) and/or a light chain variable region (VL), wherein: (i) the VH comprises a heavy chain complementarity determining region 1 (VHCDR1) having an amino acid sequence of a VHCDR1 in Table 30, Table 31, Table 10, Table 11, Table 12, or Table 13 (or a sequence with no more than 1, 2, 3, or 4 mutations, e.g., substitutions, additions, or deletions), a VHCDR2 having an amino acid sequence of a VHCDR2 in Table 30, Table 31, Table 10, Table 11, Table 12, or Table 13 (or a sequence with no more than 1, 2, 3, or 4 mutations, e.g., substitutions, additions, or deletions), and/or a VHCDR3 having an amino acid sequence of a VHCDR3 in Table 30, Table 31, Table 10, Table 11, Table 12, or Table 13 (or a sequence with no more than 1, 2, 3, or 4 mutations, e.g., substitutions, additions, or deletions), (ii) the VL comprises a light chain complementarity determining region 1 (VLCDR1) having an amino acid sequence of a VLCDR1 in Table 30, Table 31, Table 10, Table 11, Table 12, or Table 13 (or a sequence with no more than 1, 2, 3, or 4 mutations, e.g., substitutions, additions, or deletions), a VLCDR2 having an amino acid sequence of a VLCDR2 in Table 30, Table 31, Table 10, Table 11, Table 12, or Table 13 (or a sequence with no more than 1, 2, 3, or 4 mutations, e.g., substitutions, additions, or deletions), and/or a VLCDR3 having an amino acid sequence of a VLCDR3 in Table 30, Table 31, Table 10, Table 11, Table 12, or Table 13 (or a sequence with no more than 1, 2, 3, or 4 mutations, e.g., substitutions, additions, or deletions); (b) a heavy chain variable region (VH) and/or a light chain variable region (VL), wherein: (i) the VH comprises a heavy chain complementarity determining region 1 (VHCDR1) amino acid sequence of SEQ ID NO: 3 (or a sequence with no more than 1, 2, 3, or 4 mutations, e.g., substitutions, additions, or deletions), a VHCDR2 amino acid sequence of SEQ ID NO: 4 (or a sequence with no more than 1, 2, 3, or 4 mutations, e.g., substitutions, additions, or deletions), and/or a VHCDR3 amino acid sequence of SEQ ID NO: 5 (or a sequence with no more than 1, 2, 3, or 4 mutations, e.g., substitutions, additions, or deletions), and/or (ii) the VL comprises a light chain complementarity determining region 1 (VLCDR1) amino acid sequence of SEQ ID NO: 6 (or a sequence with no more than 1, 2, 3, or 4 mutations, e.g., substitutions, additions, or deletions), a VLCDR2 amino acid sequence of SEQ ID NO: 7 (or a sequence with no more than 1, 2, 3, or 4 mutations, e.g., substitutions, additions, or deletions), and/or a VLCDR3 amino acid sequence of SEQ ID NO: 8 (or a sequence with no more than 1, 2, 3, or 4 mutations, e.g., substitutions, additions, or deletions); (c) a heavy chain variable region (VH) and/or a light chain variable region (VL), wherein: (i) the VH comprises a heavy chain complementarity determining region 1 (VHCDR1) amino acid sequence of SEQ ID NO: 45 (or a sequence with no more than 1, 2, 3, or 4 mutations, e.g., substitutions, additions, or deletions), a VHCDR2 amino acid sequence of SEQ ID NO: 46 (or a sequence with no more than 1, 2, 3, or 4 mutations, e.g., substitutions, additions, or deletions), and/or a VHCDR3 amino acid sequence of SEQ ID NO: 47 (or a sequence with no more than 1, 2, 3, or 4 mutations, e.g., substitutions, additions, or deletions), and/or (ii) the VL comprises a light chain complementarity determining region 1 (VLCDR1) amino acid sequence of SEQ ID NO: 51 (or a sequence with no more than 1, 2, 3, or 4 mutations, e.g., substitutions, additions, or deletions), a VLCDR2 amino acid sequence of SEQ ID NO: 52 (or a sequence with no more than 1, 2, 3, or 4 mutations, e.g., substitutions, additions, or deletions), and/or a VLCDR3 amino acid sequence of SEQ ID NO: 53 (or a sequence with no more than 1, 2, 3, or 4 mutations, e.g., substitutions, additions, or deletions); and/or (d) a heavy chain variable region (VH) and/or a light chain variable region (VL), wherein: (i) the VH comprises a heavy chain complementarity determining region 1 (VHCDR1) amino acid sequence of SEQ ID NO: 48 (or a sequence with no more than 1, 2, 3, or 4 mutations, e.g., substitutions, additions, or deletions), a VHCDR2 amino acid sequence of SEQ ID NO: 49 (or a sequence with no more than 1, 2, 3, or 4 mutations, e.g., substitutions, additions, or deletions), and/or a VHCDR3 amino acid sequence of SEQ ID NO: 50 (or a sequence with no more than 1, 2, 3, or 4 mutations, e.g., substitutions, additions, or deletions), and/or (ii) the VL comprises a light chain complementarity determining region 1 (VLCDR1) amino acid sequence of SEQ ID NO: 54 (or a sequence with no more than 1, 2, 3, or 4 mutations, e.g., substitutions, additions, or deletions), a VLCDR2 amino acid sequence of SEQ ID NO: 55 (or a sequence with no more than 1, 2, 3, or 4 mutations, e.g., substitutions, additions, or deletions), and/or a VLCDR3 amino acid sequence of SEQ ID NO: 56 (or a sequence with no more than 1, 2, 3, or 4 mutations, e.g., substitutions, additions, or deletions).

In some embodiments, the second antigen binding domain comprises: (a) a heavy chain variable region (VH) and/or a light chain variable region (VL), wherein: (i) the VH comprises the amino acid sequence of a VH in Table 30, Table 31, Table 10, Table 11, Table 12, or Table 13 (or an amino acid sequence having at least about 77%, 80%, 85%, 90%, 95%, or 99% sequence identity thereto), and/or (ii) the VL comprises the amino acid sequence of a VL in Table 30, Table 31, Table 10, Table 11, Table 12, or Table 13 (or an amino acid sequence having at least about 77%, 80%, 85%, 90%, 95%, or 99% sequence identity thereto) (iii) the VH comprises the amino acid sequence of SEQ ID NO: 9 (or an amino acid sequence having at least about 77%, 80%, 85%, 90%, 95%, or 99% sequence identity thereto), and/or (iv) the VL comprises the amino acid sequence of SEQ ID NO: 10 (or an amino acid sequence having at least about 77%, 80%, 85%, 90%, 95%, or 99% sequence identity thereto); (b) a heavy chain variable region (VH) and/or a light chain variable region (VL), wherein: (i) the VH comprises the amino acid sequence of SEQ ID NO: 9 (or an amino acid sequence having at least about 77%, 80%, 85%, 90%, 95%, or 99% sequence identity thereto), and/or (ii) the VL comprises the amino acid sequence of SEQ ID NO: 11 (or an amino acid sequence having at least about 77%, 80%, 85%, 90%, 95%, or 99% sequence identity thereto); and/or (c) a heavy chain variable region (VH) and/or a light chain variable region (VL), wherein: (i) the VH comprises the amino acid sequence of SEQ ID NO: 1312 (or an amino acid sequence having at least about 77%, 80%, 85%, 90%, 95%, or 99% sequence identity thereto), and/or (ii) the VL comprises the amino acid sequence of SEQ ID NO: 1314 (or an amino acid sequence having at least about 77%, 80%, 85%, 90%, 95%, or 99% sequence identity thereto).

In some embodiments, the second antigen binding domain comprises: (a) a heavy chain variable region (VH) and/or a light chain variable region (VL), wherein: (i) the VH comprises a heavy chain complementarity determining region 1 (VHCDR1) amino acid sequence of SEQ ID NO: 17 (or a sequence with no more than 1, 2, 3, or 4 mutations, e.g., substitutions, additions, or deletions), a VHCDR2 amino acid sequence of SEQ ID NO: 18 (or a sequence with no more than 1, 2, 3, or 4 mutations, e.g., substitutions, additions, or deletions), and/or a VHCDR3 amino acid sequence of SEQ ID NO: 19 (or a sequence with no more than 1, 2, 3, or 4 mutations, e.g., substitutions, additions, or deletions), and/or (ii) the VL comprises a light chain complementarity determining region 1 (VLCDR1) amino acid sequence of SEQ ID NO: 20 (or a sequence with no more than 1, 2, 3, or 4 mutations, e.g., substitutions, additions, or deletions), a VLCDR2 amino acid sequence of SEQ ID NO: 21 (or a sequence with no more than 1, 2, 3, or 4 mutations, e.g., substitutions, additions, or deletions), and/or a VLCDR3 amino acid sequence of SEQ ID NO: 22 (or a sequence with no more than 1, 2, 3, or 4 mutations, e.g., substitutions, additions, or deletions); (b) a heavy chain variable region (VH) and/or a light chain variable region (VL), wherein: (i) the VH comprises a heavy chain complementarity determining region 1 (VHCDR1) amino acid sequence of SEQ ID NO: 57 (or a sequence with no more than 1, 2, 3, or 4 mutations, e.g., substitutions, additions, or deletions), a VHCDR2 amino acid sequence of SEQ ID NO: 58 (or a sequence with no more than 1, 2, 3, or 4 mutations, e.g., substitutions, additions, or deletions), and/or a VHCDR3 amino acid sequence of SEQ ID NO: 59 (or a sequence with no more than 1, 2, 3, or 4 mutations, e.g., substitutions, additions, or deletions), and/or (ii) the VL comprises a light chain complementarity determining region 1 (VLCDR1) amino acid sequence of SEQ ID NO: 63 (or a sequence with no more than 1, 2, 3, or 4 mutations, e.g., substitutions, additions, or deletions), a VLCDR2 amino acid sequence of SEQ ID NO: 64 (or a sequence with no more than 1, 2, 3, or 4 mutations, e.g., substitutions, additions, or deletions), and/or a VLCDR3 amino acid sequence of SEQ ID NO: 65 (or a sequence with no more than 1, 2, 3, or 4 mutations, e.g., substitutions, additions, or deletions); and/or (c) a heavy chain variable region (VH) and/or a light chain variable region (VL), wherein: (i) the VH comprises a heavy chain complementarity determining region 1 (VHCDR1) amino acid sequence of SEQ ID NO: 60 (or a sequence with no more than 1, 2, 3, or 4 mutations, e.g., substitutions, additions, or deletions), a VHCDR2 amino acid sequence of SEQ ID NO: 61 (or a sequence with no more than 1, 2, 3, or 4 mutations, e.g., substitutions, additions, or deletions), and/or a VHCDR3 amino acid sequence of SEQ ID NO: 62 (or a sequence with no more than 1, 2, 3, or 4 mutations, e.g., substitutions, additions, or deletions), and/or (ii) the VL comprises a light chain complementarity determining region 1 (VLCDR1) amino acid sequence of SEQ ID NO: 66 (or a sequence with no more than 1, 2, 3, or 4 mutations, e.g., substitutions, additions, or deletions), a VLCDR2 amino acid sequence of SEQ ID NO: 67 (or a sequence with no more than 1, 2, 3, or 4 mutations, e.g., substitutions, additions, or deletions), and/or a VLCDR3 amino acid sequence of SEQ ID NO: 68 (or a sequence with no more than 1, 2, 3, or 4 mutations, e.g., substitutions, additions, or deletions).

In some embodiments, the second antigen binding domain comprises: (a) a heavy chain variable region (VH) and/or a light chain variable region (VL), wherein: (i) the VH comprises the amino acid sequence of SEQ ID NO: 15 (or an amino acid sequence having at least about 77%, 80%, 85%, 90%, 95%, or 99% sequence identity thereto), and/or (ii) the VL comprises the amino acid sequence of SEQ ID NO: 16 (or an amino acid sequence having at least about 77%, 80%, 85%, 90%, 95%, or 99% sequence identity thereto); and/or (b) a heavy chain variable region (VH) and/or a light chain variable region (VL), wherein: (i) the VH comprises: the amino acid sequence of SEQ ID NO: 23 (or an amino acid sequence having at least about 77%, 80%, 85%, 90%, 95%, or 99% sequence identity thereto), the amino acid sequence of SEQ ID NO: 24 (or an amino acid sequence having at least about 77%, 80%, 85%, 90%, 95%, or 99% sequence identity thereto), or the amino acid sequence of SEQ ID NO: 25 (or an amino acid sequence having at least about 77%, 80%, 85%, 90%, 95%, or 99% sequence identity thereto); and/or (ii) the VL comprises: the amino acid sequence of SEQ ID NO: 26 (or an amino acid sequence having at least about 77%, 80%, 85%, 90%, 95%, or 99% sequence identity thereto), the amino acid sequence of SEQ ID NO: 27 (or an amino acid sequence having at least about 77%, 80%, 85%, 90%, 95%, or 99% sequence identity thereto), the amino acid sequence of SEQ ID NO: 28 (or an amino acid sequence having at least about 77%, 80%, 85%, 90%, 95%, or 99% sequence identity thereto), the amino acid sequence of SEQ ID NO: 29 (or an amino acid sequence having at least about 77%, 80%, 85%, 90%, 95%, or 99% sequence identity thereto), or the amino acid sequence of SEQ ID NO: 30 (or an amino acid sequence having at least about 77%, 80%, 85%, 90%, 95%, or 99% sequence identity thereto).

In some embodiments, the multifunctional molecule as described herein comprises: a first polypeptide comprising, e.g., from N-terminus to C-terminus, a first VL and a first CL, a second polypeptide comprising, e.g., from N-terminus to C-terminus, a first VH, a first CH1, a first dimerization domain (e.g., a first Fc), and a first moiety that binds to TCR (e.g., TCRVβ) (e.g., a first scFv that binds to TCR (e.g., TCRVβ), a third polypeptide comprising, e.g., from N-terminus to C-terminus, a second VH, a second CH1, a second dimerization domain (e.g., a second Fc), and optionally a second moiety that binds to TCR (e.g., TCRVβ) (e.g., a second scFv that binds to TCR (e.g., TCRV 0), a fourth polypeptide comprising, e.g., from N-terminus to C-terminus, a second VL and a second CL, wherein: the first VL and the first VH form a first antigen binding domain that binds to a first calreticulin protein, and the second VL and the second VH form a third antigen binding domain that binds to a second calreticulin protein, optionally wherein the first and second calreticulin proteins comprise the amino acid sequence of SEQ ID NO: 6285, 1001, or 6286, optionally wherein the first and second calreticulin mutant proteins are each independently chosen from: a molecule comprising the amino acid sequence of SEQ ID NO: 6313, or a molecule comprising the amino acid sequence of SEQ ID NO: 6314, optionally wherein the multifunctional molecule comprises the configuration of FIG. 3A or 3B.

In some embodiments, the second antigen binding domain binds to NKp30.

In some embodiments, the second antigen binding domain is chosen from an antibody molecule, e.g., an antigen binding domain, or ligand that binds to (e.g., activates) NKp30, e.g., the second antigen binding domain is an antibody molecule or ligand that binds to (e.g., activates) NKp30.

In some embodiments, the second antigen binding domain comprises: a heavy chain variable region (VH) comprising a heavy chain complementarity determining region 1 (VHCDR1) having an amino acid sequence of a VHCDR1 of Table 7, Table 35, Table 9, Table 10, or Table 34 (or a sequence with no more than 1, 2, 3, or 4 mutations, e.g., substitutions, additions, or deletions), a VHCDR2 having an amino acid sequence of a VHCDR2 of Table 7, Table 35, Table 9, Table 10, or Table 34 (or a sequence with no more than 1, 2, 3, or 4 mutations, e.g., substitutions, additions, or deletions), and/or a VHCDR3 having an amino acid sequence of a VHCDR3 of Table 7, Table 35, Table 9, Table 10, or Table 34 (or a sequence with no more than 1, 2, 3, or 4 mutations, e.g., substitutions, additions, or deletions), and/or (ii) a light chain variable region (VL) comprising a light chain complementarity determining region 1 (VLCDR1) having an amino acid sequence of a VLCDR1 of Table 8, Table 36, Table 9, Table 10, or Table 34 (or a sequence with no more than 1, 2, 3, or 4 mutations, e.g., substitutions, additions, or deletions), a VLCDR2 having an amino acid sequence of a VLCDR2 of Table 8, Table 36, Table 9, Table 10, or Table 34 (or a sequence with no more than 1, 2, 3, or 4 mutations, e.g., substitutions, additions, or deletions), and/or a VLCDR3 having an amino acid sequence of a VLCDR3 of Table 8, Table 36, Table 9, Table 10, or Table 34 (or a sequence with no more than 1, 2, 3, or 4 mutations, e.g., substitutions, additions, or deletions).

In some embodiments, the second antigen binding domain comprises: (i) a heavy chain variable region (VH) comprising a heavy chain complementarity determining region 1 (VHCDR1) amino acid sequence of SEQ ID NO: 7313 (or a sequence with no more than 1, 2, 3, or 4 mutations, e.g., substitutions, additions, or deletions), a VHCDR2 amino acid sequence of SEQ ID NO: 6001 (or a sequence with no more than 1, 2, 3, or 4 mutations, e.g., substitutions, additions, or deletions, and/or a VHCDR3 amino acid sequence of SEQ ID NO: 7315 (or a sequence with no more than 1, 2, 3, or 4 mutations, e.g., substitutions, additions, or deletions; and/or (ii) a light chain variable region (VL) comprising a light chain complementarity determining region 1 (VLCDR1) amino acid sequence of SEQ ID NO: 7326 (or a sequence with no more than 1, 2, 3, or 4 mutations, e.g., substitutions, additions, or deletions), a VLCDR2 amino acid sequence of SEQ ID NO: 7327 (or a sequence with no more than 1, 2, 3, or 4 mutations, e.g., substitutions, additions, or deletions), and/or a VLCDR3 amino acid sequence of SEQ ID NO: 7329 (or a sequence with no more than 1, 2, 3, or 4 mutations, e.g., substitutions, additions, or deletions).

In some embodiments, the second antigen binding domain comprises: (i) a VH comprising the amino acid sequence of any of SEQ ID NOs: 7298 or 7300-7304 (or an amino acid sequence having at least about 75%, 80%, 85%, 90%, 95%, or 99% sequence identity to any of SEQ ID NOs: 7298 or 7300-7304); and/or (ii) a VL comprising the amino acid sequence of any of SEQ ID NOs: 7299 or 7305-7309 (or an amino acid sequence having at least about 93%, 95%, or 99% sequence identity to any of SEQ ID NOs: 7299 or 7305-7309).

In some embodiments, the second antigen binding domain comprises: (i) a VH comprising the amino acid sequence of SEQ ID NO: 7302 (or an amino acid sequence having at least about 75%, 80%, 85%, 90%, 95%, or 99% sequence identity to 7302), and a VL comprising the amino acid sequence of SEQ ID NO: 7305 (or an amino acid sequence having at least about 75%, 80%, 85%, 90%, 95%, or 99% sequence identity to 7305); or (ii) a VH comprising the amino acid sequence of SEQ ID NO: 7302 (or an amino acid sequence having at least about 75%, 80%, 85%, 90%, 95%, or 99% sequence identity to 7302), and a VL comprising the amino acid sequence of SEQ ID NO: 7309 (or an amino acid sequence having at least about 75%, 80%, 85%, 90%, 95%, or 99% sequence identity to 7309).

In some embodiments, the second antigen binding domain comprises: (i) an amino acid sequence of SEQ ID NO: 7310 (or an amino acid sequence having at least about 75%, 80%, 85%, 90%, 95%, or 99% sequence identity to 7310); or (ii) an amino acid sequence of SEQ ID NO: 7311 (or an amino acid sequence having at least about 75%, 80%, 85%, 90%, 95%, or 99% sequence identity to 7311).

In some embodiments, the second antigen binding domain comprises: (i) a heavy chain variable region (VH) comprising a heavy chain complementarity determining region 1 (VHCDR1) amino acid sequence of SEQ ID NO: 6000, a VHCDR2 amino acid sequence of SEQ ID NO: 6001, and/or a VHCDR3 amino acid sequence of SEQ ID NO: 6002, and (ii) a light chain variable region (VL) comprising a light chain complementarity determining region 1 (VLCDR1) amino acid sequence of SEQ ID NO: 6063, a VLCDR2 amino acid sequence of SEQ ID NO: 6064, and/or a VLCDR3 amino acid sequence of SEQ ID NO: 7293.

In some embodiments, the second antigen binding domain comprises: (1) a heavy chain variable region (VH) comprising a heavy chain framework region 1 (VHFWR1) having an amino acid sequence of a VHFWR1 of Table 7, Table 35, Table 9, Table 10, or Table 34 (or a sequence with no more than 1, 2, 3, 4, 5, or 6 mutations, e.g., substitutions, additions, or deletions, therefrom), a VHFWR2 having an amino acid sequence of a VHFWR2 of Table 7, Table 35, Table 9, Table 10, or Table 34 (or a sequence with no more than 1, 2, 3, 4, 5, or 6 mutations, e.g., substitutions, additions, or deletions, therefrom), a VHFWR3 having an amino acid sequence of a VHFWR3 of Table 7, Table 35, Table 9, Table 10, or Table 34 (or a sequence with no more than 1, 2, 3, 4, 5, or 6 mutations, e.g., substitutions, additions, or deletions, therefrom), or a VHFWR4 having an amino acid sequence of a VHFWR4 of Table 7, Table 35, Table 9, Table 10, or Table 34 (or a sequence with no more than 1, 2, 3, 4, 5, or 6 mutations, e.g., substitutions, additions, or deletions, therefrom), and/or (2) a light chain variable region (VL) comprising a light chain framework region 1 (VLFWR1) having an amino acid sequence of a VLFWR1 of Table 8, Table 36, Table 9, Table 10, or Table 34 (or a sequence with no more than 1, 2, 3, 4, 5, or 6 mutations, e.g., substitutions, additions, or deletions, therefrom), a VLFWR2 having an amino acid sequence of a VLFWR2 of Table 8, Table 36, Table 9, Table 10, or Table 34 (or a sequence with no more than 1, 2, 3, 4, 5, or 6 mutations, e.g., substitutions, additions, or deletions, therefrom), a VLFWR3 having an amino acid sequence of a VLFWR3 of Table 8, Table 36, Table 9, Table 10, or Table 34 (or a sequence with no more than 1, 2, 3, 4, 5, or 6 mutations, e.g., substitutions, additions, or deletions, therefrom), or a VLFWR4 having an amino acid sequence of a VLFWR4 of Table 8, Table 36, Table 9, Table 10, or Table 34 (or a sequence with no more than 1, 2, 3, 4, 5, or 6 mutations, e.g., substitutions, additions, or deletions, therefrom).

In some embodiments, the second antigen binding domain comprises: (1) a heavy chain variable region (VH) comprising a heavy chain framework region 1 (VHFWR1) amino acid sequence of SEQ ID NO: 6003, a VHFWR2 amino acid sequence of SEQ ID NO: 6004, a VHFWR3 amino acid sequence of SEQ ID NO: 6005, or a VHFWR4 amino acid sequence of SEQ ID NO: 6006, and (3) a light chain variable region (VL) comprising a light chain framework region 1 (VLFWR1) amino acid sequence of SEQ ID NO: 6066, a VLFWR2 amino acid sequence of SEQ ID NO: 6067, a VLFWR3 amino acid sequence of SEQ ID NO: 7292, or a VLFWR4 amino acid sequence of SEQ ID NO: 6069.

In some embodiments, the second antigen binding domain comprises: (i) a VH comprising the amino acid sequence of a VH of Table 7, Table 35, Table 9, Table 10, or Table 34 (or an amino acid sequence having at least about 75%, 80%, 85%, 90%, 95%, or 99% sequence identity thereto), and/or (ii) a VL comprising the amino acid sequence of a VL of Table 8, Table 36, Table 9, Table 10, or Table 34 (or an amino acid sequence having at least about 93%, 95%, or 99% sequence identity thereto).

In some embodiments, the second antigen binding domain comprises a heavy chain comprising the amino acid sequence of a heavy chain of Table 10 (or an amino acid sequence having at least about 75%, 80%, 85%, 90%, 95%, or 99% sequence identity thereto).

In some embodiments, the second antigen binding domain comprises a light chain comprising the amino acid sequence of a light chain of Table 10 (or an amino acid sequence having at least about 75%, 80%, 85%, 90%, 95%, or 99% sequence identity thereto).

In some embodiments, the second antigen binding domain comprises a heavy chain comprising the amino acid sequence of a heavy chain of Table 10 (or an amino acid sequence having at least about 75%, 80%, 85%, 90%, 95%, or 99% sequence identity thereto), and a light chain comprising the amino acid sequence of a light chain of Table 10 (or an amino acid sequence having at least about 75%, 80%, 85%, 90%, 95%, or 99% sequence identity thereto).

In some embodiments, the multifunctional molecule as described herein comprises: a first polypeptide comprising, e.g., from N-terminus to C-terminus, a first VL and a first CL, a second polypeptide comprising, e.g., from N-terminus to C-terminus, a first VH, a first CH1, a first dimerization domain (e.g., a first Fc), and a first moiety that binds to NKp30 (e.g., a first antibody molecule or ligand that binds to NKp30), a third polypeptide comprising, e.g., from N-terminus to C-terminus, a second VH, a second CH1, a second dimerization domain (e.g., a second Fc), and optionally a second moiety that binds to NKp30 (e.g., a second antibody molecule or ligand that binds to NKp30), a fourth polypeptide comprising, e.g., from N-terminus to C-terminus, a second VL and a second CL, wherein: the first VL and the first VH form a first antigen binding domain that binds to a first calreticulin protein, and the second VL and the second VH from a third antigen binding domain that binds to a second calreticulin protein, optionally wherein the first and second calreticulin proteins comprise the amino acid sequence of SEQ ID NO: 6285, 1001, or 6286, optionally wherein the first and second calreticulin mutant proteins are each independently chosen from: a molecule comprising the amino acid sequence of SEQ ID NO: 6313, or a molecule comprising the amino acid sequence of SEQ ID NO: 6314, optionally wherein the multifunctional molecule comprises the configuration of FIG. 3A or 3B.

In some embodiments, the calreticulin protein comprises an amino acid sequence chosen from SEQ ID NOs: 6285-6312 or 1001, optionally wherein the calreticulin protein comprises an amino acid sequence chosen from SEQ ID NOs: 6313-6346 or 1002-1003.

In some embodiments, the calreticulin protein comprises the amino acid sequence of SEQ ID NO: 6285 or 1001.

In some embodiments, the calreticulin protein comprises the amino acid sequence of SEQ ID NO: 6286.

In some embodiments, the first antigen binding domain binds to an epitope located within the C-terminus of the calreticulin protein, optionally wherein the first antigen binding domain binds to an epitope located within the amino acid sequence of SEQ ID NO: 6285, 1001, or 6286.

In some embodiments, the multifunctional molecule as described herein further comprises a third antigen binding domain that binds to a second calreticulin protein, e.g., wherein the second calreticulin mutant protein comprises the amino acid sequence of SEQ ID NO: 6285, 1001, or 6286, optionally wherein: (i) the third antigen binding domain is different from the first antigen binding domain, or (ii) the third antigen binding domain is the same as the first antigen binding domain.

In some embodiments, the second calreticulin molecule is the same as the calreticulin molecule bound by the first antigen binding domain.

In some embodiments, the second calreticulin molecule is different from the calreticulin molecule bound by the first antigen binding domain.

In some embodiments, the second calreticulin protein comprises an amino acid sequence chosen from SEQ ID NOs: 6285-6312 or 1001, optionally wherein the second calreticulin protein comprises an amino acid sequence chosen from SEQ ID NOs: 6313-6346 or 1002-1003.

In some embodiments, the calreticulin protein bound by the first antigen binding domain comprises the amino acid sequence of SEQ ID NO: 6285 or 1001, and the second calreticulin protein comprises the amino acid sequence of SEQ ID NO: 6286.

In some embodiments, the third antigen binding domain binds to an epitope located within the C-terminus of the second calreticulin protein, optionally wherein the third antigen binding domain binds to an epitope located within the amino acid sequence of SEQ ID NO: 6285, 1001, or 6286.

In some embodiments, the first antigen binding domain comprises: (i) a heavy chain variable region (VH) comprising a heavy chain complementarity determining region 1 (VHCDR1) having an amino acid sequence of a VHCDR1 in Table 4, Table 24, Table 25, or Table 17 (or a sequence with no more than 1, 2, 3, or 4 mutations, e.g., substitutions, additions, or deletions), a VHCDR2 having an amino acid sequence of a VHCDR2 in Table 4, Table 24, Table 25, or Table 17 (or a sequence with no more than 1, 2, 3, or 4 mutations, e.g., substitutions, additions, or deletions), and/or a VHCDR3 having an amino acid sequence of a VHCDR3 in Table 4, Table 24, Table 25, or Table 17 (or a sequence with no more than 1, 2, 3, or 4 mutations, e.g., substitutions, additions, or deletions); (ii) a light chain variable region (VL) comprising a light chain complementarity determining region 1 (VLCDR1) having an amino acid sequence of a VLCDR1 in Table 5, Table 24, Table 25, or Table 18 (or a sequence with no more than 1, 2, 3, or 4 mutations, e.g., substitutions, additions, or deletions), a VLCDR2 having an amino acid sequence of a VLCDR2 in Table 5, Table 24, Table 25, or Table 18 (or a sequence with no more than 1, 2, 3, or 4 mutations, e.g., substitutions, additions, or deletions), and/or a VLCDR3 having an amino acid sequence of a VLCDR3 in Table 5, Table 24, Table 25, or Table 18 (or a sequence with no more than 1, 2, 3, or 4 mutations, e.g., substitutions, additions, or deletions); (iii) a VH comprising the amino acid sequence of a VH in Table 24, Table 25, or Table 16 (or an amino acid sequence having at least about 77%, 80%, 85%, 90%, 95%, or 99% sequence identity thereto); (iv) a VL comprising the amino acid sequence of a VL in Table 24, Table 25, or Table 16 (or an amino acid sequence having at least about 93%, 95%, or 99% sequence identity thereto); (v) a VH comprising a heavy chain framework region 1 (VHFWR1) having an amino acid sequence of a VHFWR1 in Table 4 or Table 6 (or a sequence with no more than 1, 2, 3, 4, 5, 6, 7, 8, or 9 mutations, e.g., substitutions, additions, or deletions), a VHFWR2 having an amino acid sequence of a VHFWR2 in Table 4 or Table 6 (or a sequence with no more than 1, 2, 3, 4, 5, 6, 7, 8, or 9 mutations, e.g., substitutions, additions, or deletions), a VHFWR3 having an amino acid sequence of a VHFWR3 in Table 4 or Table 6 (or a sequence with no more than 1, 2, 3, 4, 5, 6, 7, 8, or 9 mutations, e.g., substitutions, additions, or deletions), and/or a VHFWR4 having an amino acid sequence of a VHFWR4 in Table 4 or Table 6 (or a sequence with no more than 1, 2, 3, 4, 5, 6, 7, 8, or 9 mutations, e.g., substitutions, additions, or deletions), and/or (vi) a VL comprising a light chain framework region 1 (VLFWR1) having an amino acid sequence of a VLFWR1 in Table 5 or Table 6 (or a sequence with no more than 1, 2, 3, 4, 5, 6, 7, 8, or 9 mutations, e.g., substitutions, additions, or deletions), a VLFWR2 having an amino acid sequence of a VLFWR2 in Table 5 or Table 6 (or a sequence with no more than 1, 2, 3, 4, 5, 6, 7, 8, or 9 mutations, e.g., substitutions, additions, or deletions), a VLFWR3 having an amino acid sequence of a VLFWR3 in Table 5 or Table 6 (or a sequence with no more than 1, 2, 3, 4, 5, 6, 7, 8, or 9 mutations, e.g., substitutions, additions, or deletions), and/or a VLFWR4 having an amino acid sequence of a VLFWR4 in Table 5 or Table 6 (or a sequence with no more than 1, 2, 3, 4, 5, 6, 7, 8, or 9 mutations, e.g., substitutions, additions, or deletions).

In some embodiments, the multifunctional molecule further comprises a tumor-targeting moiety.

In some embodiments, the tumor-targeting moiety binds to a tumor antigen.

In some embodiments, the tumor antigen is selected from G6B, CD34, CD41, P-selectin, Clec2, cKIT, FLT3, MPL, ITGB3, ITGB2, GP5, GP6, GP9, GP1BA, DSC2, FCGR2A, TNFRSF10A, TNFRSF10B, or TM4SF1.

In some embodiments, the tumor-targeting moiety comprises an antibody molecule, e.g., that binds to a tumor antigen selected from G6B, CD34, CD41, P-selectin, Clec2, cKIT, FLT3, MPL, ITGB3, ITGB2, GP5, GP6, GP9, GP1BA, DSC2, FCGR2A, TNFRSF10A, TNFRSF10B, or TM4SF1.

In some embodiments, the tumor-targeting moiety comprises a VH and/or VL sequence, e.g., as listed in Table 38 or Table 20.

In some embodiments, the multifunctional molecule preferentially binds to a myeloproliferative neoplasm cell over a non-tumor cell, optionally wherein the binding between the multifunctional molecule and the myeloproliferative neoplasm cell is more than 10, 20, 30, 40, 50-fold greater than the binding between the multifunctional molecule and a non-tumor cell.

In some embodiments, the myeloproliferative neoplasm cell is chosen from a myelofibrosis cell, an essential thrombocythemia cell, a polycythemia vera cell, or a chronic myeloid cancer cell, optionally wherein: the myeloproliferative neoplasm cell does not comprise a JAK2 V617F mutation, or the myeloproliferative neoplasm cell does not comprise a MPL mutation.

In some embodiments, the multifunctional molecule as described herein further comprises a linker, e.g., a linker between the first antigen binding domain and the second antigen binding domain.

In some embodiments, the linker is chosen from: a cleavable linker, a non-cleavable linker, a peptide linker, a flexible linker, a rigid linker, a helical linker, or a non-helical linker.

In some embodiments, the linker is a peptide linker.

In some embodiments, the peptide linker comprises Gly and Ser.

In some embodiments, the peptide linker comprises an amino acid sequence chosen from SEQ ID NOs: 6214-6217 or 6220-6221 and 77-78.

In another aspect, provides herein is a nucleic acid molecule encoding the multifunctional molecule as described herein.

In another aspect, provides herein is a vector, e.g., an expression vector, comprising the nucleic acid molecule as described herein.

In another aspect, provides herein is a cell comprising the nucleic acid molecule as described herein or the vector as described herein.

In another aspect, provides herein is a method of making, e.g., producing, the multifunctional molecule as described herein, comprising culturing the cell as described herein, under suitable conditions, e.g., conditions suitable for gene expression and/or homo- or heterodimerization.

In another aspect, provides herein is a pharmaceutical composition comprising the composition as described herein, the multifunctional molecule as described herein, the nucleic acid molecule as described herein, the vector as described herein, or the cell as described herein, and a pharmaceutically acceptable carrier, excipient, diluent, or stabilizer.

In another aspect, provides herein is a method of treating a cancer, comprising administering to a subject in need thereof the composition as described herein, the multifunctional molecule as described herein, the nucleic acid molecule as described herein, the vector as described herein, the cell as described herein, or the pharmaceutical composition as described herein, wherein the multifunctional molecule is administered in an amount effective to treat the cancer.

In another aspect, provides herein is a use of the composition as described herein, the multifunctional molecule as described herein, the nucleic acid molecule as described herein, the vector as described herein, or the cell as described herein for the manufacture of a medicament for treating a cancer.

In some embodiments, the subject has cancer cells that express the first and/or second calreticulin protein.

In some embodiments, the subject has the JAK2 V617F mutation.

In some embodiments, the subject does not have the JAK2 V617F mutation.

In some embodiments, the subject has a MPL mutation.

In some embodiments, the subject does not have a MPL mutation.

In some embodiments, the cancer is a hematological cancer, optionally wherein the cancer is a myeloproliferative neoplasm, e.g., primary or idiopathic myelofibrosis (MF), essential thrombocytosis (ET), polycythemia vera (PV), or chronic myelogenous leukemia (CML), optionally wherein the cancer is myelofibrosis.

In some embodiments, the cancer is a solid tumor cancer.

In some embodiments, the method as described herein or the use as described herein further comprises administering a second therapeutic treatment.

In some embodiments, the second therapeutic treatment comprises a therapeutic agent (e.g., a chemotherapeutic agent, a biologic agent, hormonal therapy), radiation, or surgery.

In some embodiments, the therapeutic agent is selected from: a chemotherapeutic agent, or a biologic agent.

In another aspect, provides herein is a method of detecting calreticulin (e.g., wild-type and/or mutant calreticulin) in a sample or subject, comprising: contacting the sample or subject with an anti-calreticulin (e.g., wild-type and/or mutant calreticulin) antibody molecule described herein; and detecting formation of a complex between the antibody molecule and the sample or subject, thereby detecting calreticulin (e.g., wild-type and/or mutant calreticulin).

In some embodiments, calreticulin (e.g., wild-type and/or mutant calreticulin) is detected in vitro or in vivo.

In some embodiments, the method as described herein further comprises contacting a reference sample or subject with the antibody molecule; and detecting formation of a complex between the antibody molecule and the reference sample or subject, wherein a change, e.g., a statistically significant change, in the formation of the complex in the sample or subject, relative to the reference sample or subject is indicative of the presence of calreticulin (e.g., wild-type and/or mutant calreticulin) in the sample or subject.

In some embodiments, the method as described herein further comprises obtaining a sample from a subject.

In some embodiments, sample comprises one or more of plasma, tissue (e.g., cancerous tissue), biopsy, blood (e.g., whole blood), PBMCs, bone marrow, and/or lymphatic tissue, e.g., lymph node.

In some embodiments, the sample has not been frozen and/or fixed.

In some embodiments, the sample has been frozen (e.g., snap frozen) and/or fixed (e.g., formalin-fixed paraffin-embedded (FFPE)).

In some embodiments, the subject has, or is at risk of having, a disease or disorder described herein (e.g., cancer, e.g., myelofibrosis).

In some embodiments, the method as described herein further comprises performing a flow analysis, e.g., using a multi-panel method.

In some embodiments, the method as described herein further comprises assessing T-cell clonality, e.g., to determine the presence and/or level of T cell malignancy.

In some embodiments, the method as described herein further comprises measuring the level of calreticulin+(e.g., wild-type calreticulin+ and/or mutant calreticulin+) cells from the biological sample (e.g., determining if the calreticulin+ cells are depleted, e.g., relative to a reference sample or subject).

In some embodiments, the method as described herein further comprises measuring the intracellular level of calreticulin (e.g., wild-type and/or mutant calreticulin).

In some embodiments, the method as described herein further comprises measuring the membrane level of calreticulin (e.g., wild-type and/or mutant calreticulin).

In some embodiments, the method as described herein further comprises evaluating the subject for a change in prognosis, severity, or presence or absence of a disease or disorder (e.g., cancer, e.g., myelofibrosis), e.g., after treatment (e.g., with an antibody molecule described herein).

In some embodiments, the antibody molecule is detectably labeled.

In another aspect, provides herein is a method of evaluating a subject, comprising: contacting a sample (e.g., a sample described herein) from the subject with an anti-calreticulin (e.g., wild-type and/or mutant calreticulin) antibody molecule described herein; and detecting formation of a complex between the antibody molecule and the sample, thereby evaluating the subject.

In some embodiments, the subject has, or is at risk of having, a disease or disorder described herein (e.g., cancer, e.g., myelofibrosis).

In some embodiments, the subject has not been treated with an antibody molecule described herein.

In some embodiments, the subject has been treated with an antibody molecule described herein.

In another aspect, provides herein is a kit comprising an anti-calreticulin (e.g., wild-type and/or mutant calreticulin) antibody molecule described herein and instructions for use in a method of detecting calreticulin (e.g., wild-type and/or mutant calreticulin) in a sample or subject.

Those skilled in the art will recognize, or be able to ascertain using no more than routine experimentation, many equivalents to the specific embodiments of the invention described herein. Such equivalents are intended to be encompassed by the following embodiments.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and are not intended to be limiting.

Other features and advantages of the invention will be apparent from the following detailed description and claims.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1B shows the alignment of the Antibody A source mouse VH and VL framework 1, CDR 1, framework 2, CDR 2, framework 3, CDR3, and framework 4 regions with their respective humanized sequences. Kabat CDRs are shown in bold, Chothia CDRs are shown in italics, and combined CDRs are shown in boxes. The framework positions that were back mutated are double underlined. FIG. 1A shows VH sequences for murine Antibody A (SEQ ID NO: 1) and humanized Antibody A-H (SEQ ID NO: 9). FIG. 1B shows VL sequences for murine Antibody A (SEQ ID NO: 2) and humanized Antibody A-H (SEQ ID NO: 10 and SEQ ID NO: 11).

FIGS. 2A-2B shows the alignment of the Antibody B source mouse VH and VL framework 1, CDR 1, framework 2, CDR 2, framework 3, CDR3, and framework 4 regions with their respective humanized sequences. Kabat CDRs are shown in bold, Chothia CDRs are shown in italics, and combined CDRs are shown in boxes. The framework positions that were back mutated are double underlined. FIG. 2A shows the VH sequence for murine Antibody B (SEQ ID NO: 15) and humanized VH sequences B-H. 1A to B-H.1C (SEQ ID NOs: 23-25). FIG. 2B shows the VL sequence for murine Antibody B (SEQ ID NO: 16) and humanized VL sequences B-H.1D to B-H.1H (SEQ ID NOs: 26-30).

FIG. 3 depicts the phylogenetic tree of TCRBV gene family and subfamilies with corresponding antibodies mapped. Subfamily identities are as follows: Subfamily A: TCRβ V6; Subfamily B: TCRβ V10; Subfamily C: TCRβ V12; Subfamily D: TCRβ V5; Subfamily E: TCRβ V7; Subfamily F: TCRβ V11; Subfamily G: TCRβ V14; Subfamily H: TCRβ V16; Subfamily I: TCRβ V18; Subfamily J: TCRβ V9; Subfamily K: TCRβ V13; Subfamily L: TCRβ V4; Subfamily M: TCRβ V3; Subfamily N: TCRβ V2; Subfamily O: TCRβ V15; Subfamily P: TCRβ V30; Subfamily Q: TCRβ V19; Subfamily R: TCRβ V27; Subfamily S: TCRβ V28; Subfamily T: TCRβ V24; Subfamily U: TCRβ V20; Subfamily V: TCRβ V25; and Subfamily W: TCRβ V29 subfamily. Subfamily members are described in detail herein in the Section titled “TCR beta V (TCRβV)”.

FIGS. 4A-4C show human CD3+ T cells activated by anti-TCR Vβ13.1 antibody (A-H.1) for 6-days. Human CD3+ T cells were isolated using magnetic-bead separation (negative selection) and activated with immobilized (plate-coated) anti-TCR Vβ13.1 (A-H.1) or anti-CD3 E (OKT3) antibodies at 100 nM for 6 days. FIG. 4A shows two scatter plots (left: activated with OKT3; and right: activated with A-H.1) of expanded T cells assessed for TCR Vβ13.1 surface expression using anti-TCR Vβ13.1 (A-H.1) followed by a secondary fluorochrome-conjugated antibody for flow cytometry analysis. FIG. 4B shows percentage (%) of TCR Vβ13.1 positive T cells activated by anti-TCR Vβ13.1 (A-H.1) or anti-CD3e (OKT3) plotted against total T cells (CD3+). FIG. 4C shows relative cell count acquired by counting the number of events in each T cell subset gate (CD3 or TCR Vβ13.1) for 20 seconds at a constant rate of 60 μl/min. Data shown as mean value from 3 donors.

FIGS. 5A-5B show cytolytic activity of human CD3+ T cells activated by anti-TCR Vβ13.1 antibody (A-H.1) against transformed cell line RPMI 8226. FIG. 5A depicts target cell lysis of human CD3+ T cells activated with A-H.1 or OKT3. Human CD3+ T cells were isolated using magnetic-bead separation (negative selection) and activated with immobilized (plate-coated) A-H.1 or OKT3 at the indicated concentrations for 4 days prior to co-culture with RPMI 8226 cells at a (E:T) ratio of 5:1 for 2 days. Samples were next analyzed for cell lysis of RPMI 8226 cells by FACS staining for CFSE/CD138-labeled, and membrane-impermeable DNA dyes (DRAQ7) using flow cytometry analysis. FIG. 5B shows target cell lysis of human CD3+ T cells activated with A-H.1 or OKT3 incubated with RPMI-8226 at a (E:T) ratio of 5:1 for 6 days followed by cell lysis analysis of RPMI 8226 cells as described above. Percentage (%) target cell lysis was determined by normalizing to basal target cell lysis (i.e. without antibody treatment) using the following formula, [(x−basal)/(100%−basal), where x is cell lysis of sample]. Data shown is a representative of n=1 donor.

FIGS. 6A-6B show IFNg production by human PBMCs activated with the indicated antibodies. Human PBMCs were isolated from whole blood from the indicated number of donors, followed by solid-phase (plate-coated) stimulation with the indicated antibodies at 100 Nm. Supernatant was collected on Days 1, 2, 3, 5, or 6. FIG. 6A is a graph comparing the production of IFNg in human PBMCs activated with the antibodies indicated activated with anti-TCR Vβ13.1 antibodies (A-H.1 or A-H.2) or anti-CD3e antibodies (OKT3 or SP34-2) on Day 1, 2, 3, 5, or 6 post-activation. FIG. 6B shows IFNg production in human PBMCs activated with the antibodies indicated activated with the indicated anti-TCR Vβ13.1 antibodies or anti-CD3e antibody (OKT3) on Day 1, 2, 3, 5, or 6 post-activation.

FIGS. 7A-7B show IL-2 production by human PBMCs activated with the indicated antibodies. A similar experimental setup as described for FIGS. 6A-6B was used.

FIGS. 8A-8B show IL-6 production by human PBMCs activated with the indicated antibodies. A similar experimental setup as described for FIGS. 6A-6B was used.

FIGS. 9A-9B show TNF-alpha production by human PBMCs activated with the indicated antibodies. A similar experimental setup as described for FIGS. 6A-6B was used.

FIGS. 10A-10B show IL-1beta production by human PBMCs activated with the indicated antibodies. A similar experimental setup as described for FIGS. 6A-6B was used.

FIGS. 11A-11B are graphs showing delayed kinetics of IFNg secretion in human PMBCs activated by anti-TCR Vβ13.1 antibody A-H.1 when compared to PBMCs activated by anti-CD3e antibody OKT3. FIG. 11A shows IFNg secretion data from 4 donors. FIG. 11B shows IFNg secretion data from 4 additional donors. Data shown is representative of n=8 donors.

FIG. 12 depicts increased CD8+ TSCM and Temra T cell subsets in human PBMCs activated by anti-TCR Vβ13.1 antibodies (A-H.1 or A-H.2) compared to PBMCs activated by anti-CD3e antibodies (OKT3 or SP34-2).

FIGS. 13A-13F show characterization of an anti-TCRVb antibody. FIG. 13A is a graph depicting proliferation of T cells activated with anti-CD3 (OKT3) antibody or anti-TCRVb antibody. FIG. 13B shows selective expansion of CD45RA+ effector memory CD8+ and CD4+ T cells (TEMRA) cells with anti-TCRVb antibodies. Tn=naïve T cell; Tscm=stem cell memory T cell; Tcm=central memory T cell; Tem=effector memory T cell; Temra=effector memory CD45RA+ T cell. FIG. 13C is a graph showing IFN-g secretion by PBMCs stimulated with an anti-TCRVb antibody, or anti-CD3 antibodies. FIG. 13D shows target cell lysis by T cells stimulated with an anti-TCRVb antibody, or anti-CD3 antibodies. Cells were stimulated for 4 days followed by 2 days incubation with multiple myeloma target cells for assessment of cell killing. FIG. 13E is a graph showing perforin secretion by T cells stimulated with an anti-TCRVb antibody, or an anti-CD3 antibody. Perforin was analyzed by FACS staining in TCRVB-positive and TCRVB-negative T cells in PBMCs after 5 days of stimulation with 100 ng/ml plate-bound antibody. FIG. 13F is a graph showing Granzyme B by T cells stimulated with an anti-TCRVb antibody, or an anti-CD3 antibody. Granzyme B was analyzed by FACS staining in TCRVB-positive and TCRVB-negative T cells in PBMCs after 5 days of stimulation with 100 ng/ml plate-bound antibody.

FIGS. 14A-14B show production of IL-2 and IL-15 and expansion of human NK cells by stimulation of PBMCs with anti-TCRVb antibody for 6 days at a dose of 100 nM. FIG. 14A shows secretion of IL-2 or IL-15 in T cells stimulated with an anti-TCRVb antibody, or anti-CD3 antibodies. FIG. 14B depicts flow cytometry dot plots showing NKp46 staining vs CD56 antibody staining in cells stimulated with an anti-TCRVb antibody or an anti-CD3 antibody or a control sample.

FIGS. 15A-15C show secretion of cytokines in PBMCs stimulated with an anti-TCRVb antibody, or anti-CD3 antibodies.

FIGS. 16A-16B show killing of MM cells by dual targeting BCMA-TCRvb antibody molecules. FIG. 16A shows in vitro killing by one of the following dual-targeting antibody molecules: BCMA-TCRVb, BCMA-CD3, or Control-TCRVb; or an isotype control. FIG. 16B shows in vivo killing of MM cells by a dual-targeting BCM-TCRVb antibody.

FIG. 17 shows lysis of MM target cells with a dual targeting antibody which recognized FcRH5 on one arm and TCRVb on the other arm.

FIGS. 18A-18C are schematic representations of exemplary formats and configurations of functional moieties attached to a dimerization module, e.g., an immunoglobulin constant domain. FIG. 18A depicts moieties A, B, C and D, covalently linked to a heterodimeric Fc domain. FIG. 18B depicts moieties A, B, C and D, covalently linked to a homodimeric Fc domain. FIG. 18C depicts moieties A, B, C and D, covalently linked to heterodimeric heavy and light constant domains (e.g., a Fab CH₁ and a Fab CL). In some embodiments, the functional moiety is an antigen binding domain that binds to a calreticulin protein (e.g., a wild-type calreticulin protein and/or a calreticulin mutant protein). In some embodiments, the functional moiety is an antigen binding domain that binds to a wild-type calreticulin protein and a calreticulin mutant protein with approximately the same affinity. In some embodiments, the functional moiety is an antigen binding domain that preferentially binds to a calreticulin mutant protein over a wild type calreticulin protein, e.g., wherein the first calreticulin mutant protein comprises the amino acid sequence of SEQ ID NO: 6286 and the wild type calreticulin protein comprises the amino acid sequence of SEQ ID NO: 6285 or 1001. In some embodiments, the functional moiety is an immune cell engager chosen from a T cell engager, an NK cell engager, a B cell engager, a dendritic cell engager, or a macrophage cell engager. In some embodiments, the functional moiety is a cytokine molecule. In some embodiments, the functional moiety is a stromal modifying moiety.

FIGS. 19A and 19B are schematic representations of exemplary formats and configurations of a multifunctional molecule comprising a first antigen binding domain (e.g., a first Fab) that binds to a calreticulin protein (e.g., a wild-type calreticulin protein and/or a calreticulin mutant protein), a second antigen binding domain (e.g., a second Fab) that binds to a calreticulin protein (e.g., a wild-type calreticulin protein and/or a calreticulin mutant protein), and one or more moieties that bind to CD3 (e.g., an scFv that binds to CD3). In one embodiment, the first antigen binding domain (e.g., the first Fab) binds to a calreticulin protein (e.g., a wild-type calreticulin protein and/or a calreticulin mutant protein) disclosed herein, e.g., a calreticulin mutant protein disclosed in Table 2 or 3, e.g., Type 1 or Type 2 calreticulin mutant protein disclosed in Table 2 or 3, e.g., a calreticulin mutant protein comprising the amino acid sequence of SEQ ID NO: 6113 or 6314. In one embodiment, the second antigen binding domain (e.g., the second Fab) binds to a calreticulin protein (e.g., a wild-type calreticulin protein and/or a calreticulin mutant protein) disclosed herein, e.g., a calreticulin mutant protein disclosed in Table 2 or 3, e.g., Type 1 or Type 2 calreticulin mutant protein disclosed in Table 2 or 3, e.g., a calreticulin mutant protein comprising the amino acid sequence of SEQ ID NO: 6313 or 6314.

FIGS. 20A and 20B are schematic representations of exemplary formats and configurations of a multifunctional molecule comprising a first antigen binding domain (e.g., a first Fab) that binds to a calreticulin protein (e.g., a wild-type calreticulin protein and/or a calreticulin mutant protein), a second antigen binding domain (e.g., a second Fab) that binds to a calreticulin protein (e.g., a wild-type calreticulin protein and/or a calreticulin mutant protein), and one or more moieties that bind to TCR (e.g., TCR p) (e.g., an scFv that binds to TCR (e.g., TCR P)). In one embodiment, the first antigen binding domain (e.g., the first Fab) binds to a calreticulin protein (e.g., a wild-type calreticulin protein and/or a calreticulin mutant protein) disclosed herein, e.g., a calreticulin mutant protein disclosed in Table 2 or 3, e.g., Type 1 or Type 2 calreticulin mutant protein disclosed in Table 2 or 3, e.g., a calreticulin mutant protein comprising the amino acid sequence of SEQ ID NO: 6313 or 6314. In one embodiment, the second antigen binding domain (e.g., the second Fab) binds to a calreticulin protein (e.g., a wild-type calreticulin protein and/or a calreticulin mutant protein) disclosed herein, e.g., a calreticulin mutant protein disclosed in Table 2 or 3, e.g., Type 1 or Type 2 calreticulin mutant protein disclosed in Table 2 or 3, e.g., a calreticulin mutant protein comprising the amino acid sequence of SEQ ID NO: 6313 or 6314.

FIGS. 21A and 21B are schematic representations of exemplary formats and configurations of a multifunctional molecule comprising a first antigen binding domain (e.g., a first Fab) that binds to a calreticulin protein (e.g., a wild-type calreticulin protein and/or a calreticulin mutant protein), a second antigen binding domain (e.g., a second Fab) that binds to a calreticulin protein (e.g., a wild-type calreticulin protein and/or a calreticulin mutant protein), and one or more moieties that bind to NKp30 (e.g., an antibody molecule or ligand that binds to NKp30). In one embodiment, the first antigen binding domain (e.g., the first Fab) binds to a calreticulin protein (e.g., a wild-type calreticulin protein and/or a calreticulin mutant protein) disclosed herein, e.g., a calreticulin mutant protein disclosed in Table 2 or 3, e.g., Type 1 or Type 2 calreticulin mutant protein disclosed in Table 2 or 3, e.g., a calreticulin mutant protein comprising the amino acid sequence of SEQ ID NO: 6313 or 6314. In one embodiment, the second antigen binding domain (e.g., the second Fab) binds to a calreticulin protein (e.g., a wild-type calreticulin protein and/or a calreticulin mutant protein) disclosed herein, e.g., a calreticulin mutant protein disclosed in Table 2 or 3, e.g., Type 1 or Type 2 calreticulin mutant protein disclosed in Table 2 or 3, e.g., a calreticulin mutant protein comprising the amino acid sequence of SEQ ID NO: 6313 or 6314.

FIG. 22 is a graph showing binding of NKp30 antibodies to NK92 cells. Data was calculated as the percent-AF747 positive population.

FIG. 23 is a graph showing activation of NK92 cells by NKp30 antibodies. Data were generated using hamster anti-NKp30 mAbs.

FIGS. 24A-24D are schematics showing exemplary multispecific molecules comprising a TGFβ inhibitor. In some embodiments, the TGFβ inhibitor comprises a TGF-beta receptor ECD homodimer. In some embodiments, the TGFβ inhibitor comprises a TGFBR2 ECD heterodimer. In FIGS. 24A and 24B, the two TGFBR ECD domains are linked to the C-terminus of two Fc regions. In some embodiments, the CH1-Fc-TGFBR ECD region shown in FIG. 24A or 24B comprises the amino acid sequence of SEQ ID NO: 6405 or 3193. In some embodiments, the Fc-TGFBR ECD region shown in FIG. 24A or 24B comprises the amino acid sequence of SEQ ID NO: 6407 or 6408. In FIGS. 24C and 24D, the two TGFBR ECD domains are linked to CH1 and CL, respectively. In some embodiments, the TGFBR ECD-CH1-Fc region shown in FIG. 24C or 24D comprises the amino acid sequence of SEQ ID NO: 6409 or 6410. In some embodiments, the TGFBR ECD-CL region shown in FIG. 24C or 24D comprises the amino acid sequence of SEQ ID NO: 6411 or 6412. In some embodiments, the multispecific molecule comprises a binding moiety A and a binding moiety B. In some embodiments, the binding moiety A or binding moiety B is a calreticulin-targeting antigen binding domain disclosed herein.

FIGS. 25A-25B are a series of graphs showing enzyme-linked immunosorbent assay (ELISA) results showing the level of binding of the parental IgG form of antibody 6C10 (BKM0106) to wild-type calreticulin (CALR WT) and two calreticulin mutants (CALR ins and CALR del, as described herein). FIG. 25A shows ELISA results when the indicated antigen (CALR WT, CALR ins, or CALR del) was coated on the plate. FIG. 25B shows ELISA results when the BKM0106 antibody was coated on the plate.

FIGS. 26A-26B are a series of graphs showing binding of the parental IgG form of antibody 6C10 (BKM0106) to cells expressing one of two calreticulin mutants (CALR ins and CALR del, as described herein), as assessed by FACS.

FIG. 27 is a graph showing therapeutic efficacy of various antibody molecules in an in vivo murine model of myelofibrosis. Antibody molecules tested included ADCC-enabled antibody molecules against mutant calreticulin (mtCalR), bispecific antibodies comprising a mtCalR-binding domain and a second binding domain specific to another target (i.e., TCRvβ or CD3) and an LALAPG variant Fc region. Naïve mouse spleen and vehicle were used as controls.

FIG. 28 is a table showing in vitro binding of exemplary anti-CD3 antibody molecules BKM0020, BKM0025, BKM0028, BKM0038, as described herein, to human CD3e (huCD3e) and cynomolgus CD3e (cCD3e).

FIG. 29 is a graph showing binding of exemplary anti-CD3 antibody molecule BKM0020, as described herein, to Jurkat cells expressing human CD3e (huCD3e).

FIGS. 30A and 30B are schematics showing the alignments of affinity matured humanized Antibody A-H sequences. FIG. 30A shows the alignment of affinity matured humanized Antibody A-H VL sequences (SEQ ID NOs: 3377-3389, respectively, in order of appearance). FIG. 30B shows the alignment of affinity matured humanized Antibody A-H VH sequences (SEQ ID NOS 3390-3436, respectively, in order of appearance).

### DETAILED DESCRIPTION OF THE INVENTION

Disclosed herein are multifunctional molecules (also referred to herein as “multispecific molecules”) that include a plurality of (e.g., two or more) functionalities (or binding specificities), comprising (i) an antigen binding domain that binds to a calreticulin protein (e.g., a wild-type calreticulin protein and/or a calreticulin mutant protein), e.g., wherein the calreticulin protein comprises the amino acid sequence of SEQ ID NO: 6285, 1001, or 6286, and (ii) one, two, or all of: (a) an immune cell engager chosen from a T cell engager, an NK cell engager, a B cell engager, a dendritic cell engager, or a macrophage cell engager; (b) a cytokine molecule; (c) a stromal modifying moiety, and (d) a tumor-targeting moiety (e.g., which binds to a tumor antigen chosen from: G6B, CD34, CD41, P-selectin, Clec2, cKIT, FLT3, MPL, ITGB3, ITGB2, GP5, GP6, GP9, GP1BA, DSC2, FCGR2A, TNFRSF10A, TNFRSF10B, or TM4SF1). In some embodiments, the antigen binding domain binds to a calreticulin protein (e.g., a wild-type calreticulin protein or a mutant calreticulin protein, e.g., as described herein). In some embodiments, the antigen binding domain binds to a calreticulin mutant protein disclosed in Table 2 or Table 3. In some embodiments, the antigen binding domain binds to Type 1 calreticulin mutant protein disclosed in Table 2 or Table 3. In some embodiments, the antigen binding domain binds to Type 2 calreticulin mutant protein disclosed in Table 2 or Table 3. In some embodiments, the antigen binding domain binds to both Type 1 and Type 2 calreticulin mutant proteins disclosed in Table 2 or Table 3. In some embodiments, the T cell engager comprises an additional antigen binding domain that binds to the variable chain of the beta subunit of TCR (TCR PV), e.g., a TCR p V6 or TCR p V12.

In an embodiment, the multispecific or multifunctional molecule is a bispecific (or bifunctional) molecule, a trispecific (or trifunctional) molecule, or a tetraspecific (or tetrafunctional) molecule. In an embodiment, the multispecific or multifunctional molecule is a bispecific molecule.

Without being bound by theory, the multispecific or multifunctional molecules disclosed herein are expected to localize (e.g., bridge) and/or activate an immune cell (e.g., an immune effector cell chosen from a T cell, an NK cell, a B cell, a dendritic cell or a macrophage), in the presence of a cell expressing the calreticulin protein, e.g., on the surface. Increasing the proximity and/or activity of the immune cell, in the presence of the cell expressing the calreticulin protein, using the multispecific or multifunctional molecules described herein is expected to enhance an immune response against the target cell, thereby providing a more effective therapy.

Novel multifunctional, e.g., multispecific, molecules that include (i) a stromal modifying moiety and (ii) an antigen binding domain that binds to a calreticulin protein (e.g., a wild-type calreticulin protein and/or a calreticulin mutant protein), e.g., wherein the calreticulin protein comprises the amino acid sequence of SEQ ID NO: 6285, 1001, or 6286 are disclosed. Without being bound by theory, the multifunctional molecules disclosed herein are believed to inter alia target (e.g., localize to) a cancer site, and alter the tumor stroma, e.g., alter the tumor microenvironment near the cancer site. The multifunctional molecules can further include one or both of: an immune cell engager (e.g., chosen from one, two, three, or all of a T cell engager, NK cell engager, a B cell engager, a dendritic cell engager, or a macrophage cell engager); and/or a cytokine molecule. Accordingly, provided herein are, inter alia, multifunctional, e.g., multispecific molecules, that include the aforesaid moieties, nucleic acids encoding the same, methods of producing the aforesaid molecules, and methods of treating a cancer using the aforesaid molecules.

Accordingly, provided herein are, inter alia, multispecific or multifunctional molecules (e.g., multispecific or multifunctional antibody molecules) that include the aforesaid moieties, nucleic acids encoding the same, methods of producing the aforesaid molecules, and methods of treating a disease or disorder, e.g., cancer, using the aforesaid molecules.

### Definitions

In some embodiments, the multifunctional molecule includes an immune cell engager. “An immune cell engager” refers to one or more binding specificities that bind and/or activate an immune cell, e.g., a cell involved in an immune response. In some embodiments, the immune cell is chosen from a T cell, an NK cell, a B cell, a dendritic cell, and/or the macrophage cell. The immune cell engager can be an antibody molecule, a receptor molecule (e.g., a full length receptor, receptor fragment, or fusion thereof (e.g., a receptor-Fc fusion)), or a ligand molecule (e.g., a full length ligand, ligand fragment, or fusion thereof (e.g., a ligand-Fc fusion)) that binds to the immune cell antigen (e.g., the T cell, the NK cell antigen, the B cell antigen, the dendritic cell antigen, and/or the macrophage cell antigen). In some embodiments, the immune cell engager specifically binds to the target immune cell, e.g., binds preferentially to the target immune cell. For example, when the immune cell engager is an antibody molecule, it binds to an immune cell antigen (e.g., a T cell antigen, an NK cell antigen, a B cell antigen, a dendritic cell antigen, and/or a macrophage cell antigen) with a dissociation constant of less than about 10 nM.

As used herein, the terms “T cell receptor beta variable chain,” “TCRVβ,” “TCRVb,” and “TCRβV” are used interchangeably to refer to an extracellular region of the T cell receptor beta chain which comprises the antigen recognition domain of the T cell receptor. The term TCRVβ or TCRβV includes isoforms, mammalian, e.g., human TCRBV, species homologs of human and analogs comprising at least one common epitope with TCRBV. Human TCRβV comprises a gene family comprising subfamilies including, but not limited to: a TCRβ V6 subfamily, a TCRβ V10 subfamily, a TCRβ V12 subfamily, a TCRβ V5 subfamily, a TCRβ V7 subfamily, a TCRβ V11 subfamily, a TCRβ V14 subfamily, a TCRβ V16 subfamily, a TCRβ V18 subfamily, a TCRβ V9 subfamily, a TCRβ V13 subfamily, a TCRβ V4 subfamily, a TCRβ V3 subfamily, a TCRβ V2 subfamily, a TCRβ V15 subfamily, a TCRβ V30 subfamily, a TCRβ V19 subfamily, a TCRβ V27 subfamily, a TCRβ V28 subfamily, a TCRβ V24 subfamily, a TCRβ V20 subfamily, TCRβ V25 subfamily, or a TCRβ V29 subfamily. In some embodiments, the TCRβ V6 subfamily comprises: TCRβ V6-4*01, TCRβ V6-4*02, TCRβ V6-9*01, TCRβ V6-8*01, TCRβ V6-5*01, TCRβ V6-6*02, TCRβ V6-6*01, TCRβ V6-2*01, TCRβ V6-3*01 or TCRβ V6-1*01. In some embodiments, TCRβV comprises TCRβ V6-5*01. TCRβ V6-5*01 is also known as TRBV65; TCRBV6S5; TCRBV13S1, or TCRβ V13.1. The amino acid sequence of TCRβ V6-5*01, e.g., human TCRβ V6-5*01, is known in that art, e.g., as provided by IMGT ID L36092. In some embodiments, TCRβ V6-5*01 is encoded by the nucleic acid sequence of SEQ ID NO: 1043, or a sequence having 85%, 90%, 95%, 99% or more identity thereof. In some embodiments, TCRβ V6-5*01 comprises the amino acid sequence of SEQ ID NO: 1044, or a sequence having 85%, 90%, 95%, 99% or more identity thereof.

In some embodiments, the multifunctional molecule includes a cytokine molecule. As used herein, a “cytokine molecule” refers to full length, a fragment or a variant of a cytokine; a cytokine further comprising a receptor domain, e.g., a cytokine receptor dimerizing domain; or an agonist of a cytokine receptor, e.g., an antibody molecule (e.g., an agonistic antibody) to a cytokine receptor, that elicits at least one activity of a naturally-occurring cytokine. In some embodiments the cytokine molecule is chosen from interleukin-2 (IL-2), interleukin-7 (IL-7), interleukin-12 (IL-12), interleukin-15 (IL-15), interleukin-18 (IL-18), interleukin-21 (IL-21), or interferon gamma, or a fragment or variant thereof, or a combination of any of the aforesaid cytokines. The cytokine molecule can be a monomer or a dimer. In some embodiments, the cytokine molecule can further include a cytokine receptor dimerizing domain. In other embodiments, the cytokine molecule is an agonist of a cytokine receptor, e.g., an antibody molecule (e.g., an agonistic antibody) to a cytokine receptor chosen from an IL-15Ra or IL-21R.

As used herein, the term “molecule” as used in, e.g., antibody molecule, cytokine molecule, receptor molecule, includes full-length, naturally-occurring molecules, as well as variants, e.g., functional variants (e.g., truncations, fragments, mutated (e.g., substantially similar sequences) or derivatized form thereof), so long as at least one function and/or activity of the unmodified (e.g., naturally-occurring) molecule remains.

In some embodiments, the multifunctional molecule includes a stromal modifying moiety. A “stromal modifying moiety,” as used herein refers to an agent, e.g., a protein (e.g., an enzyme), that is capable of altering, e.g., degrading a component of, the stroma. In some embodiments, the component of the stroma is chosen from, e.g., an ECM component, e.g., a glycosaminoglycan, e.g., hyaluronan (also known as hyaluronic acid or HA), chondroitin sulfate, chondroitin, dermatan sulfate, heparin sulfate, heparin, entactin, tenascin, aggrecan and keratin sulfate; or an extracellular protein, e.g., collagen, laminin, elastin, fibrinogen, fibronectin, and vitronectin.

Certain terms are defined below.

As used herein, the articles “a” and “an” refer to one or more than one, e.g., to at least one, of the grammatical object of the article. The use of the words “a” or “an” when used in conjunction with the term “comprising” herein may mean “one,” but it is also consistent with the meaning of “one or more,” “at least one,” and “one or more than one.”

As used herein, “about” and “approximately” generally mean an acceptable degree of error for the quantity measured given the nature or precision of the measurements. Exemplary degrees of error are within 20 percent (%), typically, within 10%, and more typically, within 5% of a given range of values.

“Antibody molecule” as used herein refers to a protein, e.g., an immunoglobulin chain or fragment thereof, comprising at least one immunoglobulin variable domain sequence. An antibody molecule encompasses antibodies (e.g., full-length antibodies) and antibody fragments. In an embodiment, an antibody molecule comprises an antigen binding or functional fragment of a full-length antibody, or a full-length immunoglobulin chain. For example, a full-length antibody is an immunoglobulin (Ig) molecule (e.g., an IgG antibody) that is naturally occurring or formed by normal immunoglobulin gene fragment recombinatorial processes). In some embodiments, an antibody molecule refers to an immunologically active, antigen-binding portion of an immunoglobulin molecule, such as an antibody fragment. An antibody fragment, e.g., functional fragment, is a portion of an antibody, e.g., Fab, Fab′, F(ab′)₂, F(ab)₂, variable fragment (Fv), domain antibody (dAb), or single chain variable fragment (scFv). A functional antibody fragment binds to the same antigen as that recognized by the intact (e.g., full-length) antibody. The terms “antibody fragment” or “functional fragment” also include isolated fragments consisting of the variable regions, such as the “Fv” fragments consisting of the variable regions of the heavy and light chains or recombinant single chain polypeptide molecules in which light and heavy variable regions are connected by a peptide linker (“scFv proteins”). In some embodiments, an antibody fragment does not include portions of antibodies without antigen binding activity, such as Fc fragments or single amino acid residues. Exemplary antibody molecules include full length antibodies and antibody fragments, e.g., dAb (domain antibody), single chain, Fab, Fab′, and F(ab′)₂ fragments, and single chain variable fragments (scFvs).

As used herein, an “immunoglobulin variable domain sequence” refers to an amino acid sequence which can form the structure of an immunoglobulin variable domain. For example, the sequence may include all or part of the amino acid sequence of a naturally-occurring variable domain. For example, the sequence may or may not include one, two, or more N- or C-terminal amino acids, or may include other alterations that are compatible with formation of the protein structure.

In some embodiments, an antibody molecule is monospecific, e.g., it comprises binding specificity for a single epitope. In some embodiments, an antibody molecule is multispecific, e.g., it comprises a plurality of immunoglobulin variable domain sequences, where a first immunoglobulin variable domain sequence has binding specificity for a first epitope and a second immunoglobulin variable domain sequence has binding specificity for a second epitope. In some embodiments, an antibody molecule is a bispecific antibody molecule. “Bispecific antibody molecule” as used herein refers to an antibody molecule that has specificity for more than one (e.g., two, three, four, or more) epitope and/or antigen.

“Antigen” (Ag) as used herein refers to a molecule that can provoke an immune response, e.g., involving activation of certain immune cells and/or antibody generation. Any macromolecule, including almost all proteins or peptides, can be an antigen. Antigens can also be derived from genomic recombinant or DNA. For example, any DNA comprising a nucleotide sequence or a partial nucleotide sequence that encodes a protein capable of eliciting an immune response encodes an “antigen.” In some embodiments, an antigen does not need to be encoded solely by a full-length nucleotide sequence of a gene, nor does an antigen need to be encoded by a gene at all. In some embodiments, an antigen can be synthesized or can be derived from a biological sample, e.g., a tissue sample, a tumor sample, a cell, or a fluid with other biological components. As used, herein a “tumor antigen” or interchangeably, a “cancer antigen” includes any molecule present on, or associated with, a cancer, e.g., a cancer cell or a tumor microenvironment that can provoke an immune response. As used, herein an “immune cell antigen” includes any molecule present on, or associated with, an immune cell that can provoke an immune response.

The “antigen-binding site,” or “binding portion” of an antibody molecule refers to the part of an antibody molecule, e.g., an immunoglobulin (Ig) molecule, that participates in antigen binding. In some embodiments, the antigen binding site is formed by amino acid residues of the variable (V) regions of the heavy (H) and light (L) chains. Three highly divergent stretches within the variable regions of the heavy and light chains, referred to as hypervariable regions, are disposed between more conserved flanking stretches called “framework regions,” (FRs). FRs are amino acid sequences that are naturally found between, and adjacent to, hypervariable regions in immunoglobulins. In some embodiments, in an antibody molecule, the three hypervariable regions of a light chain and the three hypervariable regions of a heavy chain are disposed relative to each other in three dimensional space to form an antigen-binding surface, which is complementary to the three-dimensional surface of a bound antigen. The three hypervariable regions of each of the heavy and light chains are referred to as “complementarity-determining regions,” or “CDRs.” The framework region and CDRs have been defined and described, e.g., in Kabat, E. A., et al. (1991) Sequences of Proteins of Immunological Interest, Fifth Edition, U.S. Department of Health and Human Services, NIH Publication No. 91-3242, and Chothia, C. et al. (1987) J. Mol. Biol. 196:901-917. Each variable chain (e.g., variable heavy chain and variable light chain) is typically made up of three CDRs and four FRs, arranged from amino-terminus to carboxy-terminus in the amino acid order: FR1, CDR1, FR2, CDR2, FR3, CDR3, and FR4.

“Cancer” as used herein can encompass all types of oncogenic processes and/or cancerous growths. In some embodiments, cancer includes primary tumors as well as metastatic tissues or malignantly transformed cells, tissues, or organs. In some embodiments, cancer encompasses all histopathologies and stages, e.g., stages of invasiveness/severity, of a cancer. In some embodiments, cancer includes relapsed and/or resistant cancer. The terms “cancer” and “tumor” can be used interchangeably. For example, both terms encompass solid and liquid tumors. As used herein, the term “cancer” or “tumor” includes premalignant, as well as malignant cancers and tumors.

As used herein, an “immune cell” refers to any of various cells that function in the immune system, e.g., to protect against agents of infection and foreign matter. In some embodiments, this term includes leukocytes, e.g., neutrophils, eosinophils, basophils, lymphocytes, and monocytes. Innate leukocytes include phagocytes (e.g., macrophages, neutrophils, and dendritic cells), mast cells, eosinophils, basophils, and natural killer cells. Innate leukocytes identify and eliminate pathogens, either by attacking larger pathogens through contact or by engulfing and then killing microorganisms, and are mediators in the activation of an adaptive immune response. The cells of the adaptive immune system are special types of leukocytes, called lymphocytes. B cells and T cells are important types of lymphocytes and are derived from hematopoietic stem cells in the bone marrow. B cells are involved in the humoral immune response, whereas T cells are involved in cell-mediated immune response. The term “immune cell” includes immune effector cells.

“Immune effector cell,” as that term is used herein, refers to a cell that is involved in an immune response, e.g., in the promotion of an immune effector response. Examples of immune effector cells include, but are not limited to, T cells, e.g., alpha/beta T cells and gamma/delta T cells, B cells, natural killer (NK) cells, natural killer T (NK T) cells, and mast cells.

The term “effector function” or “effector response” refers to a specialized function of a cell. Effector function of a T cell, for example, may be cytolytic activity or helper activity including the secretion of cytokines.

The compositions and methods of the present invention encompass polypeptides and nucleic acids having the sequences specified, or sequences substantially identical or similar thereto, e.g., sequences at least 80%, 85%, 90%, 95% identical or higher to the sequence specified. In the context of an amino acid sequence, the term “substantially identical” is used herein to refer to a first amino acid that contains a sufficient or minimum number of amino acid residues that are i) identical to, or ii) conservative substitutions of aligned amino acid residues in a second amino acid sequence such that the first and second amino acid sequences can have a common structural domain and/or common functional activity. For example, amino acid sequences that contain a common structural domain having at least about 80%, 85%, 90%. 91%, 92%, 93%, 94%, 95%, 96%, 97%, 98% or 99% identity to a reference sequence, e.g., a sequence provided herein.

In the context of nucleotide sequence, the term “substantially identical” is used herein to refer to a first nucleic acid sequence that contains a sufficient or minimum number of nucleotides that are identical to aligned nucleotides in a second nucleic acid sequence such that the first and second nucleotide sequences encode a polypeptide having common functional activity, or encode a common structural polypeptide domain or a common functional polypeptide activity. For example, nucleotide sequences having at least about 80%, 85%, 90%, 91%, 92%, 93%, 94%, 95%, 96%, 97%, 98% or 99% identity to a reference sequence, e.g., a sequence provided herein.

The term “variant” refers to a polypeptide that has a substantially identical amino acid sequence to a reference amino acid sequence, or is encoded by a substantially identical nucleotide sequence. In some embodiments, the variant is a functional variant.

The term “functional variant” refers to a polypeptide that has a substantially identical amino acid sequence to a reference amino acid sequence, or is encoded by a substantially identical nucleotide sequence, and is capable of having one or more activities of the reference amino acid sequence.

Calculations of homology or sequence identity between sequences (the terms are used interchangeably herein) are performed as follows.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 30%, preferably at least 40%, more preferably at least 50%, 60%, and even more preferably at least 70%, 80%, 90%, 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”).

The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In a preferred embodiment, the percent identity between two amino acid sequences is determined using the Needleman and Wunsch ((1970) J. Mol. Biol. 48:444-453) algorithm which has been incorporated into the GAP program in the GCG software package (available at http://www.gcg.com), using either a Blossum 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package (available at http://www.gcg.com), using a NWSgapdna.CMP matrix and a gap weight of 40, 50, 60, 70, or 80 and a length weight of 1, 2, 3, 4, 5, or 6. A particularly preferred set of parameters (and the one that should be used unless otherwise specified) are a Blossum 62 scoring matrix with a gap penalty of 12, a gap extend penalty of 4, and a frameshift gap penalty of 5.

The percent identity between two amino acid or nucleotide sequences can be determined using the algorithm of E. Meyers and W. Miller ((1989) CABIOS, 4:11-17) which has been incorporated into the ALIGN program (version 2.0), using a PAM120 weight residue table, a gap length penalty of 12 and a gap penalty of 4.

The nucleic acid and protein sequences described herein can be used as a “query sequence” to perform a search against public databases to, for example, identify other family members or related sequences. Such searches can be performed using the NBLAST and XBLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to a nucleic acid molecule of the invention. BLAST protein searches can be performed with the XBLAST program, score=50, wordlength=3 to obtain amino acid sequences homologous to protein molecules of the invention. To obtain gapped alignments for comparison purposes, Gapped BLAST can be utilized as described in Altschul et al., (1997) Nucleic Acids Res. 25:3389-3402. When utilizing BLAST and Gapped BLAST programs, the default parameters of the respective programs (e.g., XBLAST and NBLAST) can be used. See http://www.ncbi.nlm.nih.gov.

It is understood that the molecules of the present invention may have additional conservative or non-essential amino acid substitutions, which do not have a substantial effect on their functions.

The term “amino acid” is intended to embrace all molecules, whether natural or synthetic, which include both an amino functionality and an acid functionality and capable of being included in a polymer of naturally-occurring amino acids. Exemplary amino acids include naturally-occurring amino acids; analogs, derivatives and congeners thereof, amino acid analogs having variant side chains; and all stereoisomers of any of any of the foregoing. As used herein the term “amino acid” includes both the D- or L-optical isomers and peptidomimetics.

A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

The terms “polypeptide”, “peptide” and “protein” (if single chain) are used interchangeably herein to refer to polymers of amino acids of any length. The polymer may be linear or branched, it may comprise modified amino acids, and it may be interrupted by non-amino acids. The terms also encompass an amino acid polymer that has been modified; for example, disulfide bond formation, glycosylation, lipidation, acetylation, phosphorylation, or any other manipulation, such as conjugation with a labeling component. The polypeptide can be isolated from natural sources, can be a produced by recombinant techniques from a eukaryotic or prokaryotic host, or can be a product of synthetic procedures.

The terms “nucleic acid,” “nucleic acid sequence,” “nucleotide sequence,” or “polynucleotide sequence,” and “polynucleotide” are used interchangeably. They refer to a polymeric form of nucleotides of any length, either deoxyribonucleotides or ribonucleotides, or analogs thereof. The polynucleotide may be either single-stranded or double-stranded, and if single-stranded may be the coding strand or non-coding (antisense) strand. A polynucleotide may comprise modified nucleotides, such as methylated nucleotides and nucleotide analogs. The sequence of nucleotides may be interrupted by non-nucleotide components. A polynucleotide may be further modified after polymerization, such as by conjugation with a labeling component. The nucleic acid may be a recombinant polynucleotide, or a polynucleotide of genomic, cDNA, semisynthetic, or synthetic origin which either does not occur in nature or is linked to another polynucleotide in a non-natural arrangement.

The term “isolated,” as used herein, refers to material that is removed from its original or native environment (e.g., the natural environment if it is naturally occurring). For example, a naturally-occurring polynucleotide or polypeptide present in a living animal is not isolated, but the same polynucleotide or polypeptide, separated by human intervention from some or all of the co-existing materials in the natural system, is isolated. Such polynucleotides could be part of a vector and/or such polynucleotides or polypeptides could be part of a composition, and still be isolated in that such vector or composition is not part of the environment in which it is found in nature.

As used herein, the term “transforming growth factor beta-1 (TGF-beta 1)” refers to a protein that in humans is encoded by the gene TGFB1, or its orthologs. Swiss-Prot accession number P01137 provides exemplary human TGF-beta 1 amino acid sequences. An exemplary immature human TGF-beta 1 amino acid sequence is provided in SEQ ID NO: 6378. An exemplary mature human TGF-beta 1 amino acid sequence is provided in SEQ ID NO: 6395.

As used herein, the term “transforming growth factor beta-2 (TGF-beta 2)” refers to a protein that in humans is encoded by the gene TGFB2, or its orthologs. Swiss-Prot accession number P61812 provides exemplary human TGF-beta 2 amino acid sequences. An exemplary immature human TGF-beta 2 amino acid sequence is provided in SEQ ID NO: 6379. An exemplary mature human TGF-beta 2 amino acid sequence is provided in SEQ ID NO: 6396.

As used herein, the term “transforming growth factor beta-3 (TGF-beta 3)” refers to a protein that in humans is encoded by the gene TGFB3, or its orthologs. Swiss-Prot accession number P10600 provides exemplary human TGF-beta 3 amino acid sequences. An exemplary immature human TGF-beta 3 amino acid sequence is provided in SEQ ID NO: 6380. An exemplary mature human TGF-beta 3 amino acid sequence is provided in SEQ ID NO: 6397.

As used herein, a “TGF-beta receptor polypeptide” refers to a TGF-beta receptor (e.g., TGFBR1, TGFBR2, or TGFBR3) or its fragment, or variant thereof.

As used herein, the term “transforming growth factor beta receptor type 1 (TGFBR1)” (also known as ALK-5 or SKR4) refers to a protein that in humans is encoded by the gene TGFBR1, or its orthologs. Swiss-Prot accession number P36897 provides exemplary human TGFBR1 amino acid sequences. Exemplary immature human TGFBR1 amino acid sequences are provided in SEQ ID NOs: 6381, 6382, and 6383. Exemplary mature human TGFBR1 amino acid sequences are provided in SEQ ID NOs: 6398, 6399, and 6400. As used herein, a “TGFBR1 polypeptide” refers to a TGFBR1 or its fragment, or variant thereof.

As used herein, the term “transforming growth factor beta receptor type 2 (TGFBR2)” refers to a protein that in humans is encoded by the gene TGFBR2, or its orthologs. Swiss-Prot accession number P37173 provides exemplary human TGFBR2 amino acid sequences. Exemplary immature human TGFBR2 amino acid sequences are provided in SEQ ID NOs: 6384 and 6385. Exemplary mature human TGFBR2 amino acid sequences are provided in SEQ ID NOs: 6401 and 6402. As used herein, a “TGFBR2 polypeptide” refers to a TGFBR2 or its fragment, or variant thereof.

As used herein, the term “transforming growth factor beta receptor type 3 (TGFBR3)” refers to a protein that in humans is encoded by the gene TGFBR3, or its orthologs. Swiss-Prot accession number Q03167 provides exemplary human TGFBR3 amino acid sequences. Exemplary immature human TGFBR3 amino acid sequences are provided in SEQ ID NOs: 6392 and 6393. Exemplary mature human TGFBR3 amino acid sequences are provided in SEQ ID NOs: 6403 and 6404. As used herein, a “TGFBR3 polypeptide” refers to a TGFBR3 or its fragment, or variant thereof.

Various aspects of the invention are described in further detail below. Additional definitions are set out throughout the specification.

### Antibody Molecules

In some embodiments, a multifunctional molecule, multispecific molecule, and/or an antigen binding domain as described herein comprises an antibody molecule. In one embodiment, the antibody molecule binds to a cancer antigen, e.g., a tumor antigen or a stromal antigen. In some embodiments, the cancer antigen is, e.g., a mammalian, e.g., a human, cancer antigen. In other embodiments, the antibody molecule binds to an immune cell antigen, e.g., a mammalian, e.g., a human, immune cell antigen. For example, the antibody molecule binds specifically to an epitope, e.g., linear or conformational epitope, on the cancer antigen or the immune cell antigen.

In an embodiment, an antibody molecule is a monospecific antibody molecule and binds a single epitope. E.g., a monospecific antibody molecule having a plurality of immunoglobulin variable domain sequences, each of which binds the same epitope.

In an embodiment an antibody molecule is a multispecific or multifunctional antibody molecule, e.g., it comprises a plurality of immunoglobulin variable domains sequences, wherein a first immunoglobulin variable domain sequence of the plurality has binding specificity for a first epitope and a second immunoglobulin variable domain sequence of the plurality has binding specificity for a second epitope. In an embodiment the first and second epitopes are on the same antigen, e.g., the same protein (or subunit of a multimeric protein). In an embodiment the first and second epitopes overlap. In an embodiment the first and second epitopes do not overlap. In an embodiment the first and second epitopes are on different antigens, e.g., the different proteins (or different subunits of a multimeric protein). In an embodiment a multispecific antibody molecule comprises a third, fourth or fifth immunoglobulin variable domain. In an embodiment, a multispecific antibody molecule is a bispecific antibody molecule, a trispecific antibody molecule, or a tetraspecific antibody molecule.

In an embodiment a multispecific antibody molecule is a bispecific antibody molecule. A bispecific antibody has specificity for no more than two antigens. A bispecific antibody molecule is characterized by a first immunoglobulin variable domain sequence which has binding specificity for a first epitope and a second immunoglobulin variable domain sequence that has binding specificity for a second epitope. In an embodiment the first and second epitopes are on the same antigen, e.g., the same protein (or subunit of a multimeric protein). In an embodiment the first and second epitopes overlap. In an embodiment the first and second epitopes do not overlap. In an embodiment the first and second epitopes are on different antigens, e.g., the different proteins (or different subunits of a multimeric protein). In an embodiment a bispecific antibody molecule comprises a heavy chain variable domain sequence and a light chain variable domain sequence which have binding specificity for a first epitope and a heavy chain variable domain sequence and a light chain variable domain sequence which have binding specificity for a second epitope. In an embodiment a bispecific antibody molecule comprises a half antibody having binding specificity for a first epitope and a half antibody having binding specificity for a second epitope. In an embodiment a bispecific antibody molecule comprises a half antibody, or fragment thereof, having binding specificity for a first epitope and a half antibody, or fragment thereof, having binding specificity for a second epitope. In an embodiment a bispecific antibody molecule comprises a scFv or a Fab, or fragment thereof, have binding specificity for a first epitope and a scFv or a Fab, or fragment thereof, have binding specificity for a second epitope.

In an embodiment, an antibody molecule comprises a diabody, and a single-chain molecule, as well as an antigen-binding fragment of an antibody (e.g., Fab, F(ab′)₂, and Fv). For example, an antibody molecule can include a heavy (H) chain variable domain sequence (abbreviated herein as VH), and a light (L) chain variable domain sequence (abbreviated herein as VL). In an embodiment an antibody molecule comprises or consists of a heavy chain and a light chain (referred to herein as a half antibody. In another example, an antibody molecule includes two heavy (H) chain variable domain sequences and two light (L) chain variable domain sequence, thereby forming two antigen binding sites, such as Fab, Fab′, F(ab′)₂, Fc, Fd, Fd′, Fv, single chain antibodies (scFv for example), single variable domain antibodies, diabodies (Dab) (bivalent and bispecific), and chimeric (e.g., humanized) antibodies, which may be produced by the modification of whole antibodies or those synthesized de novo using recombinant DNA technologies. These functional antibody fragments retain the ability to selectively bind with their respective antigen or receptor. Antibodies and antibody fragments can be from any class of antibodies including, but not limited to, IgG, IgA, IgM, IgD, and IgE, and from any subclass (e.g., IgG1, IgG2, IgG3, and IgG4) of antibodies. The a preparation of antibody molecules can be monoclonal or polyclonal. An antibody molecule can also be a human, humanized, CDR-grafted, or in vitro generated antibody. The antibody can have a heavy chain constant region chosen from, e.g., IgG1, IgG2, IgG3, or IgG4. The antibody can also have a light chain chosen from, e.g., kappa or lambda. The term “immunoglobulin” (Ig) is used interchangeably with the term “antibody” herein.

Examples of antigen-binding fragments of an antibody molecule include: (i) a Fab fragment, a monovalent fragment consisting of the VL, VH, CL and CH1 domains; (ii) a F(ab′)₂ fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a diabody (dAb) fragment, which consists of a VH domain; (vi) a camelid or camelized variable domain; (vii) a single chain Fv (scFv), see e.g., Bird et al. (1988) Science 242:423-426; and Huston et al. (1988) Proc. Natl. Acad. Sci. USA 85:5879-5883); (viii) a single domain antibody. These antibody fragments are obtained using conventional techniques known to those with skill in the art, and the fragments are screened for utility in the same manner as are intact antibodies.

Antibody molecules include intact molecules as well as functional fragments thereof. Constant regions of the antibody molecules can be altered, e.g., mutated, to modify the properties of the antibody (e.g., to increase or decrease one or more of: Fc receptor binding, antibody glycosylation, the number of cysteine residues, effector cell function, or complement function).

Antibody molecules can also be single domain antibodies. Single domain antibodies can include antibodies whose complementary determining regions are part of a single domain polypeptide. Examples include, but are not limited to, heavy chain antibodies, antibodies naturally devoid of light chains, single domain antibodies derived from conventional 4-chain antibodies, engineered antibodies and single domain scaffolds other than those derived from antibodies. Single domain antibodies may be any of the art, or any future single domain antibodies. Single domain antibodies may be derived from any species including, but not limited to mouse, human, camel, llama, fish, shark, goat, rabbit, and bovine. According to another aspect of the invention, a single domain antibody is a naturally occurring single domain antibody known as heavy chain antibody devoid of light chains. Such single domain antibodies are disclosed in WO 9404678, for example. For clarity reasons, this variable domain derived from a heavy chain antibody naturally devoid of light chain is known herein as a VHH or nanobody to distinguish it from the conventional VH of four chain immunoglobulins. Such a VHH molecule can be derived from antibodies raised in Camelidae species, for example in camel, llama, dromedary, alpaca and guanaco. Other species besides Camelidae may produce heavy chain antibodies naturally devoid of light chain; such VHHs are within the scope of the invention.

The VH and VL regions can be subdivided into regions of hypervariability, termed “complementarity determining regions” (CDR), interspersed with regions that are more conserved, termed “framework regions” (FR or FW).

The extent of the framework region and CDRs has been precisely defined by a number of methods (see, Kabat, E. A., et al. (1991) Sequences of Proteins of Immunological Interest, Fifth Edition, U.S. Department of Health and Human Services, NIH Publication No. 91-3242; Chothia, C. et al. (1987) J. Mol. Biol. 196:901-917; and the AbM definition used by Oxford Molecular's AbM antibody modeling software. See, generally, e.g., Protein Sequence and Structure Analysis of Antibody Variable Domains. In: Antibody Engineering Lab Manual (Ed.: Duebel, S. and Kontermann, R., Springer-Verlag, Heidelberg).

The terms “complementarity determining region,” and “CDR,” as used herein refer to the sequences of amino acids within antibody variable regions which confer antigen specificity and binding affinity. In general, there are three CDRs in each heavy chain variable region (HCDR1, HCDR2, HCDR3) and three CDRs in each light chain variable region (LCDR1, LCDR2, LCDR3).

The precise amino acid sequence boundaries of a given CDR can be determined using any of a number of known schemes, including those described by Kabat et al. (1991), “Sequences of Proteins of Immunological Interest,” 5th Ed. Public Health Service, National Institutes of Health, Bethesda, MD (“Kabat” numbering scheme), Al-Lazikani et al., (1997) JMB 273,927-948 (“Chothia” numbering scheme). As used herein, the CDRs defined according the “Chothia” number scheme are also sometimes referred to as “hypervariable loops.”

For example, under Kabat, the CDR amino acid residues in the heavy chain variable domain (VH) are numbered 31-35 (HCDR1), 50-65 (HCDR2), and 95-102 (HCDR3); and the CDR amino acid residues in the light chain variable domain (VL) are numbered 24-34 (LCDR1), 50-56 (LCDR2), and 89-97 (LCDR3). Under Chothia, the CDR amino acids in the VH are numbered 26-32 (HCDR1), 52-56 (HCDR2), and 95-102 (HCDR3); and the amino acid residues in VL are numbered 26-32 (LCDR1), 50-52 (LCDR2), and 91-96 (LCDR3).

Each VH and VL typically includes three CDRs and four FRs, arranged from amino-terminus to carboxy-terminus in the following order: FR1, CDR1, FR2, CDR2, FR3, CDR3, FR4.

The antibody molecule can be a polyclonal or a monoclonal antibody.

The terms “monoclonal antibody” or “monoclonal antibody composition” as used herein refer to a preparation of antibody molecules of single molecular composition. A monoclonal antibody composition displays a single binding specificity and affinity for a particular epitope. A monoclonal antibody can be made by hybridoma technology or by methods that do not use hybridoma technology (e.g., recombinant methods).

The antibody can be recombinantly produced, e.g., produced by phage display or by combinatorial methods.

Phage display and combinatorial methods for generating antibodies are known in the art (as described in, e.g., Ladner et al. U.S. Pat. No. 5,223,409; Kang et al. International Publication No. WO 92/18619; Dower et al. International Publication No. WO 91/17271; Winter et al. International Publication WO 92/20791; Markland et al. International Publication No. WO 92/15679; Breitling et al. International Publication WO 93/01288; McCafferty et al. International Publication No. WO 92/01047; Garrard et al. International Publication No. WO 92/09690; Ladner et al. International Publication No. WO 90/02809; Fuchs et al. (1991) Bio Technology 9:1370-1372; Hay et al. (1992) Hum Antibod Hybridomas 3:81-85; Huse et al. (1989) Science 246:1275-1281; Griffths et al. (1993) EMBO J 12:725-734; Hawkins et al. (1992) JMol Biol 226:889-896; Clackson et al. (1991) Nature 352:624-628; Gram et al. (1992) PNAS 89:3576-3580; Garrad et al. (1991) Bio Technology 9:1373-1377; Hoogenboom et al. (1991) Nuc Acid Res 19:4133-4137; and Barbas et al. (1991) PNAS 88:7978-7982, the contents of all of which are incorporated by reference herein).

In one embodiment, the antibody is a fully human antibody (e.g., an antibody made in a mouse which has been genetically engineered to produce an antibody from a human immunoglobulin sequence), or a non-human antibody, e.g., a rodent (mouse or rat), goat, primate (e.g., monkey), camel antibody. Preferably, the non-human antibody is a rodent (mouse or rat antibody). Methods of producing rodent antibodies are known in the art.

Human monoclonal antibodies can be generated using transgenic mice carrying the human immunoglobulin genes rather than the mouse system. Splenocytes from these transgenic mice immunized with the antigen of interest are used to produce hybridomas that secrete human mAbs with specific affinities for epitopes from a human protein (see, e.g., Wood et al. International Application WO 91/00906, Kucherlapati et al. PCT publication WO 91/10741; Lonberg et al. International Application WO 92/03918; Kay et al. International Application 92/03917; Lonberg, N. et al. 1994 Nature 368:856-859; Green, L. L. et al. 1994 Nature Genet. 7:13-21; Morrison, S. L. et al. 1994 Proc. Natl. Acad. Sci. USA 81:6851-6855; Bruggeman et al. 1993 Year Immunol 7:33-40; Tuaillon et al. 1993 PNAS 90:3720-3724; Bruggeman et al. 1991 Eur J Immunol 21:1323-1326).

An antibody molecule can be one in which the variable region, or a portion thereof, e.g., the CDRs, are generated in a non-human organism, e.g., a rat or mouse. Chimeric, CDR-grafted, and humanized antibodies are within the invention. Antibody molecules generated in a non-human organism, e.g., a rat or mouse, and then modified, e.g., in the variable framework or constant region, to decrease antigenicity in a human are within the invention.

An “effectively human” protein is a protein that does substantially not evoke a neutralizing antibody response, e.g., the human anti-murine antibody (HAMA) response. HAMA can be problematic in a number of circumstances, e.g., if the antibody molecule is administered repeatedly, e.g., in treatment of a chronic or recurrent disease condition. A HAMA response can make repeated antibody administration potentially ineffective because of an increased antibody clearance from the serum (see, e.g., Saleh et al., Cancer Immunol. Immunother., 32:180-190 (1990)) and also because of potential allergic reactions (see, e.g., LoBuglio et al., Hybridoma, 5:5117-5123 (1986)).

Chimeric antibodies can be produced by recombinant DNA techniques known in the art (see Robinson et al., International Patent Publication PCT/US86/02269; Akira, et al., European Patent Application 184,187; Taniguchi, M., European Patent Application 171,496; Morrison et al., European Patent Application 173,494; Neuberger et al., International Application WO 86/01533; Cabilly et al. U.S. Pat. No. 4,816,567; Cabilly et al., European Patent Application 125,023; Better et al. (1988 Science 240:1041-1043); Liu et al. (1987) PNAS 84:3439-3443; Liu et al., 1987, J. Immunol. 139:3521-3526; Sun et al. (1987) PNAS 84:214-218; Nishimura et al., 1987, Canc. Res. 47:999-1005; Wood et al. (1985) Nature 314:446-449; and Shaw et al., 1988, J Natl Cancer Inst. 80:1553-1559).

A humanized or CDR-grafted antibody will have at least one or two but generally all three recipient CDRs (of heavy and or light immuoglobulin chains) replaced with a donor CDR. The antibody may be replaced with at least a portion of a non-human CDR or only some of the CDRs may be replaced with non-human CDRs. It is only necessary to replace the number of CDRs required for binding to the antigen. Preferably, the donor will be a rodent antibody, e.g., a rat or mouse antibody, and the recipient will be a human framework or a human consensus framework. Typically, the immunoglobulin providing the CDRs is called the “donor” and the immunoglobulin providing the framework is called the “acceptor.” In one embodiment, the donor immunoglobulin is a non-human (e.g., rodent). The acceptor framework is a naturally-occurring (e.g., a human) framework or a consensus framework, or a sequence about 85% or higher, preferably 90%, 95%, 99% or higher identical thereto.

As used herein, the term “consensus sequence” refers to the sequence formed from the most frequently occurring amino acids (or nucleotides) in a family of related sequences (See e.g., Winnaker, From Genes to Clones (Verlagsgesellschaft, Weinheim, Germany 1987). In a family of proteins, each position in the consensus sequence is occupied by the amino acid occurring most frequently at that position in the family. If two amino acids occur equally frequently, either can be included in the consensus sequence. A “consensus framework” refers to the framework region in the consensus immunoglobulin sequence.

An antibody molecule can be humanized by methods known in the art (see e.g., Morrison, S. L., 1985, Science 229:1202-1207, by Oi et al., 1986, BioTechniques 4:214, and by Queen et al. U.S. Pat. Nos. 5,585,089, 5,693,761 and 5,693,762, the contents of all of which are hereby incorporated by reference).

Humanized or CDR-grafted antibody molecules can be produced by CDR-grafting or CDR substitution, wherein one, two, or all CDRs of an immunoglobulin chain can be replaced. See e.g., U.S. Pat. No. 5,225,539; Jones et al. 1986 Nature 321:552-525; Verhoeyan et al. 1988 Science 239:1534; Beidler et al. 1988 J. Immunol. 141:4053-4060; Winter U.S. Pat. No. 5,225,539, the contents of all of which are hereby expressly incorporated by reference. Winter describes a CDR-grafting method which may be used to prepare the humanized antibodies of the present invention (UK Patent Application GB 2188638A, filed on Mar. 26, 1987; Winter U.S. Pat. No. 5,225,539), the contents of which is expressly incorporated by reference.

Also within the scope of the invention are humanized antibody molecules in which specific amino acids have been substituted, deleted or added. Criteria for selecting amino acids from the donor are described in U.S. Pat. No. 5,585,089, e.g., columns 12-16 of U.S. Pat. No. 5,585,089, e.g., columns 12-16 of U.S. Pat. No. 5,585,089, the contents of which are hereby incorporated by reference. Other techniques for humanizing antibodies are described in Padlan et al. EP 519596 A1, published on Dec. 23, 1992.

The antibody molecule can be a single chain antibody. A single-chain antibody (scFv) may be engineered (see, for example, Colcher, D. et al. (1999) Ann N YAcad Sci 880:263-80; and Reiter, Y. (1996) Clin Cancer Res 2:245-52). The single chain antibody can be dimerized or multimerized to generate multivalent antibodies having specificities for different epitopes of the same target protein.

In yet other embodiments, the antibody molecule has a heavy chain constant region chosen from, e.g., the heavy chain constant regions of IgG1, IgG2, IgG3, IgG4, IgM, IgA1, IgA2, IgD, and IgE; particularly, chosen from, e.g., the (e.g., human) heavy chain constant regions of IgG1, IgG2, IgG3, and IgG4. In another embodiment, the antibody molecule has a light chain constant region chosen from, e.g., the (e.g., human) light chain constant regions of kappa or lambda. The constant region can be altered, e.g., mutated, to modify the properties of the antibody (e.g., to increase or decrease one or more of: Fc receptor binding, antibody glycosylation, the number of cysteine residues, effector cell function, and/or complement function). In one embodiment the antibody has: effector function; and can fix complement. In other embodiments the antibody does not; recruit effector cells; or fix complement. In another embodiment, the antibody has reduced or no ability to bind an Fc receptor. For example, it is a isotype or subtype, fragment or other mutant, which does not support binding to an Fc receptor, e.g., it has a mutagenized or deleted Fc receptor binding region.

Methods for altering an antibody constant region are known in the art. Antibodies with altered function, e.g. altered affinity for an effector ligand, such as FcR on a cell, or the C1 component of complement can be produced by replacing at least one amino acid residue in the constant portion of the antibody with a different residue (see e.g., EP 388,151 Al, U.S. Pat. Nos. 5,624,821 and 5,648,260, the contents of all of which are hereby incorporated by reference). Similar type of alterations could be described which if applied to the murine, or other species immunoglobulin would reduce or eliminate these functions.

An antibody molecule can be derivatized or linked to another functional molecule (e.g., another peptide or protein). As used herein, a “derivatized” antibody molecule is one that has been modified. Methods of derivatization include but are not limited to the addition of a fluorescent moiety, a radionucleotide, a toxin, an enzyme or an affinity ligand such as biotin. Accordingly, the antibody molecules of the invention are intended to include derivatized and otherwise modified forms of the antibodies described herein, including immunoadhesion molecules. For example, an antibody molecule can be functionally linked (by chemical coupling, genetic fusion, noncovalent association or otherwise) to one or more other molecular entities, such as another antibody (e.g., a bispecific antibody or a diabody), a detectable agent, a cytotoxic agent, a pharmaceutical agent, and/or a protein or peptide that can mediate association of the antibody or antibody portion with another molecule (such as a streptavidin core region or a polyhistidine tag).

One type of derivatized antibody molecule is produced by crosslinking two or more antibodies (of the same type or of different types, e.g., to create bispecific antibodies). Suitable crosslinkers include those that are heterobifunctional, having two distinctly reactive groups separated by an appropriate spacer (e.g., m-maleimidobenzoyl-N-hydroxysuccinimide ester) or homobifunctional (e.g., disuccinimidyl suberate). Such linkers are available from Pierce Chemical Company, Rockford, Ill.

### Multispecific or Multifunctional Antibody Molecules

Exemplary structures of multispecific and multifunctional molecules defined herein are described throughout. Exemplary structures are further described in: Weidle U et al. (2013) The Intriguing Options of Multispecific Antibody Formats for Treatment of Cancer. Cancer Genomics & Proteomics 10: 1-18 (2013); and Spiess C et al. (2015) Alternative molecular formats and therapeutic applications for bispecific antibodies. Molecular Immunology 67: 95-106; the full contents of each of which is incorporated by reference herein).

In some embodiments, multispecific antibody molecules can comprise more than one antigen-binding site, where different sites are specific for different antigens. In some embodiments, multispecific antibody molecules can bind more than one (e.g., two or more) epitopes on the same antigen. In some embodiments, multispecific antibody molecules comprise an antigen-binding site specific for a target cell (e.g., cancer cell) and a different antigen-binding site specific for an immune effector cell. In some embodiments, the multispecific antibody molecule is a bispecific, trispecific, or tetraspecific antibody molecule. In one embodiment, the multispecific antibody molecule is a bispecific antibody molecule. Bispecific antibody molecules can be classified into five different structural groups: (i) bispecific immunoglobulin G (BsIgG); (ii) IgG appended with an additional antigen-binding moiety; (iii) bispecific antibody fragments; (iv) bispecific fusion proteins; and (v) bispecific antibody conjugates.

BsIgG is a format that is monovalent for each antigen. Exemplary BsIgG formats include but are not limited to crossMab, DAF (two-in-one), DAF (four-in-one), DutaMab, DT-IgG, knobs-in-holes common LC, knobs-in-holes assembly, charge pair, Fab-arm exchange, SEEDbody, triomab, LUZ-Y, Fcab, κλ-body, orthogonal Fab. See Spiess et al. Mol. Immunol. 67(2015):95-106. Exemplary BsIgGs include catumaxomab (Fresenius Biotech, Trion Pharma, Neopharm), which contains an anti-CD3 arm and an anti-EpCAM arm; and ertumaxomab (Neovii Biotech, Fresenius Biotech), which targets CD3 and HER2. In some embodiments, BsIgG comprises heavy chains that are engineered for heterodimerization. For example, heavy chains can be engineered for heterodimerization using a “knobs-into-holes” strategy, a SEED platform, a common heavy chain (e.g., in κλ-bodies), and use of heterodimeric Fc regions. See Spiess et al. Mol. Immunol. 67(2015):95-106. Strategies that have been used to avoid heavy chain pairing of homodimers in BsIgG include knobs-in-holes, duobody, azymetric, charge pair, HA-TF, SEEDbody, and differential protein A affinity. See Id. BsIgG can be produced by separate expression of the component antibodies in different host cells and subsequent purification/assembly into a BsIgG. BsIgG can also be produced by expression of the component antibodies in a single host cell. BsIgG can be purified using affinity chromatography, e.g., using protein A and sequential pH elution.

IgG appended with an additional antigen-binding moiety is another format of bispecific antibody molecules. For example, monospecific IgG can be engineered to have bispecificity by appending an additional antigen-binding unit onto the monospecific IgG, e.g., at the N- or C-terminus of either the heavy or light chain. Exemplary additional antigen-binding units include single domain antibodies (e.g., variable heavy chain or variable light chain), engineered protein scaffolds, and paired antibody variable domains (e.g., single chain variable fragments or variable fragments). See Id. Examples of appended IgG formats include dual variable domain IgG (DVD-Ig), IgG(H)-scFv, scFv-(H)IgG, IgG(L)-scFv, scFv-(L)IgG, IgG(L,H)-Fv, IgG(H)-V, V(H)-IgG, IgG(L)-V, V(L)-IgG, KIH IgG-scFab, 2scFv-IgG, IgG-2scFv, scFv4-Ig, zybody, and DVI-IgG (four-in-one). See Spiess et al. Mol. Immunol. 67(2015):95-106. An example of an IgG-scFv is MM-141 (Merrimack Pharmaceuticals), which binds IGF-1R and HER3. Examples of DVD-Ig include ABT-981 (AbbVie), which binds IL-1α and IL-1β; and ABT-122 (AbbVie), which binds TNF and IL-17A.

Bispecific antibody fragments (BsAb) are a format of bispecific antibody molecules that lack some or all of the antibody constant domains. For example, some BsAb lack an Fc region. In some embodiments, bispecific antibody fragments include heavy and light chain regions that are connected by a peptide linker that permits efficient expression of the BsAb in a single host cell. Exemplary bispecific antibody fragments include but are not limited to nanobody, nanobody-HAS, BiTE, Diabody, DART, TandAb, scDiabody, scDiabody-CH3, Diabody-CH3, triple body, miniantibody, minibody, TriBi minibody, scFv-CH3 KIH, Fab-scFv, scFv-CH-CL-scFv, F(ab′)₂, F(ab′)₂-scFv2, scFv-KIH, Fab-scFv-Fc, tetravalent HCAb, scDiabody-Fc, Diabody-Fc, tandem scFv-Fc, and intrabody. See Id. For example, the BiTE format comprises tandem scFvs, where the component scFvs bind to CD3 on T cells and a surface antigen on cancer cells

Bispecific fusion proteins include antibody fragments linked to other proteins, e.g., to add additional specificity and/or functionality. An example of a bispecific fusion protein is an immTAC, which comprises an anti-CD3 scFv linked to an affinity-matured T-cell receptor that recognizes HLA-presented peptides. In some embodiments, the dock-and-lock (DNL) method can be used to generate bispecific antibody molecules with higher valency. Also, fusions to albumin binding proteins or human serum albumin can be extend the serum half-life of antibody fragments. See Id.

In some embodiments, chemical conjugation, e.g., chemical conjugation of antibodies and/or antibody fragments, can be used to create BsAb molecules. See Id. An exemplary bispecific antibody conjugate includes the CovX-body format, in which a low molecular weight drug is conjugated site-specifically to a single reactive lysine in each Fab arm or an antibody or fragment thereof. In some embodiments, the conjugation improves the serum half-life of the low molecular weight drug. An exemplary CovX-body is CVX-241 (NCT01004822), which comprises an antibody conjugated to two short peptides inhibiting either VEGF or Ang2. See Id.

The antibody molecules can be produced by recombinant expression, e.g., of at least one or more component, in a host system. Exemplary host systems include eukaryotic cells (e.g., mammalian cells, e.g., CHO cells, or insect cells, e.g., SF9 or S2 cells) and prokaryotic cells (e.g., E. coli). Bispecific antibody molecules can be produced by separate expression of the components in different host cells and subsequent purification/assembly. Alternatively, the antibody molecules can be produced by expression of the components in a single host cell. Purification of bispecific antibody molecules can be performed by various methods such as affinity chromatography, e.g., using protein A and sequential pH elution. In other embodiments, affinity tags can be used for purification, e.g., histidine-containing tag, myc tag, or streptavidin tag.

### CDR-Grafted Scaffolds

In some embodiments, the antibody molecule is a CDR-grafted scaffold domain. In some embodiments, the scaffold domain is based on a fibronectin domain, e.g., fibronectin type III domain. The overall fold of the fibronectin type III (Fn3) domain is closely related to that of the smallest functional antibody fragment, the variable domain of the antibody heavy chain. There are three loops at the end of Fn3; the positions of BC, DE and FG loops approximately correspond to those of CDR1, 2 and 3 of the VH domain of an antibody. Fn3 does not have disulfide bonds; and therefore Fn3 is stable under reducing conditions, unlike antibodies and their fragments (see, e.g., WO 98/56915; WO 01/64942; WO 00/34784). An Fn3 domain can be modified (e.g., using CDRs or hypervariable loops described herein) or varied, e.g., to select domains that bind to an antigen/marker/cell described herein.

In some embodiments, a scaffold domain, e.g., a folded domain, is based on an antibody, e.g., a “minibody” scaffold created by deleting three beta strands from a heavy chain variable domain of a monoclonal antibody (see, e.g., Tramontano et al., 1994, J Mol. Recognit. 7:9; and Martin et al., 1994, EMBO J. 13:5303-5309). The “minibody” can be used to present two hypervariable loops. In some embodiments, the scaffold domain is a V-like domain (see, e.g., Coia et al. WO 99/45110) or a domain derived from tendamistatin, which is a 74 residue, six-strand beta sheet sandwich held together by two disulfide bonds (see, e.g., McConnell and Hoess, 1995, J Mol. Biol. 250:460). For example, the loops of tendamistatin can be modified (e.g., using CDRs or hypervariable loops) or varied, e.g., to select domains that bind to a marker/antigen/cell described herein. Another exemplary scaffold domain is a beta-sandwich structure derived from the extracellular domain of CTLA-4 (see, e.g., WO 00/60070).

Other exemplary scaffold domains include but are not limited to T-cell receptors; MHC proteins; extracellular domains (e.g., fibronectin Type III repeats, EGF repeats); protease inhibitors (e.g., Kunitz domains, ecotin, BPTI, and so forth); TPR repeats; trifoil structures; zinc finger domains; DNA-binding proteins; particularly monomeric DNA binding proteins; RNA binding proteins; enzymes, e.g., proteases (particularly inactivated proteases), RNase; chaperones, e.g., thioredoxin, and heat shock proteins; and intracellular signaling domains (such as SH2 and SH3 domains). See, e.g., US 20040009530 and U.S. Pat. No. 7,501,121, incorporated herein by reference.

In some embodiments, a scaffold domain is evaluated and chosen, e.g., by one or more of the following criteria: (1) amino acid sequence, (2) sequences of several homologous domains, (3) 3-dimensional structure, and/or (4) stability data over a range of pH, temperature, salinity, organic solvent, oxidant concentration. In some embodiments, the scaffold domain is a small, stable protein domain, e.g., a protein of less than 100, 70, 50, 40 or 30 amino acids. The domain may include one or more disulfide bonds or may chelate a metal, e.g., zinc.

### Antibody-Based Fusions

A variety of formats can be generated which contain additional binding entities attached to the N or C terminus of antibodies. These fusions with single chain or disulfide stabilized Fvs or Fabs result in the generation of tetravalent molecules with bivalent binding specificity for each antigen. Combinations of scFvs and scFabs with IgGs enable the production of molecules which can recognize three or more different antigens.

### Antibody-Fab Fusion

Antibody-Fab fusions are bispecific antibodies comprising a traditional antibody to a first target and a Fab to a second target fused to the C terminus of the antibody heavy chain. Commonly the antibody and the Fab will have a common light chain. Antibody fusions can be produced by (1) engineering the DNA sequence of the target fusion, and (2) transfecting the target DNA into a suitable host cell to express the fusion protein. It seems like the antibody-scFv fusion may be linked by a (Gly)-Ser linker between the C-terminus of the CH3 domain and the N-terminus of the scFv, as described by Coloma, J. et al. (1997) Nature Biotech 15:159.

### Antibody-scFv Fusion

Antibody-scFv Fusions are bispecific antibodies comprising a traditional antibody and a scFv of unique specificity fused to the C terminus of the antibody heavy chain. The scFv can be fused to the C terminus through the Heavy Chain of the scFv either directly or through a linker peptide. Antibody fusions can be produced by (1) engineering the DNA sequence of the target fusion, and (2) transfecting the target DNA into a suitable host cell to express the fusion protein. It seems like the antibody-scFv fusion may be linked by a (Gly)-Ser linker between the C-terminus of the CH3 domain and the N-terminus of the scFv, as described by Coloma, J. et al. (1997) Nature Biotech 15:159.

### Variable Domain Immunoglobulin DVD

A related format is the dual variable domain immunoglobulin (DVD), which are composed of VH and VL domains of a second specificity place upon the N termini of the V domains by shorter linker sequences.

Other exemplary multispecific antibody formats include, e.g., those described in the following US20160114057A1, US20130243775A1, US20140051833, US20130022601, US20150017187A1, US20120201746A1, US20150133638A1, US20130266568A1, US20160145340A1, WO2015127158A1, US20150203591A1, US20140322221A1, US20130303396A1, US20110293613, US20130017200A1, US20160102135A1, WO2015197598A2, WO2015197582A1, U.S. Pat. No. 9,359,437, US20150018529, WO2016115274A1, WO2016087416A1, US20080069820A1, U.S. Pat. Nos. 9,145,588B, 7,919,257, and US20150232560A1. Exemplary multispecific molecules utilizing a full antibody-Fab/scFab format include those described in the following, U.S. Pat. No. 9,382,323B2, US20140072581A1, US20140308285A1, US20130165638A1, US20130267686A1, US20140377269A1, U.S. Pat. No. 7,741,446B2, and WO1995009917A1. Exemplary multispecific molecules utilizing a domain exchange format include those described in the following, US20150315296A1, WO2016087650A1, US20160075785A1, WO2016016299A1, US20160130347A1, US20150166670, U.S. Pat. No. 8,703,132B2, US20100316645, U.S. Pat. No. 8,227,577B2, US20130078249.

### Fc-Containing Entities (Mini-Antibodies)

Fc-containing entities, also known as mini-antibodies, can be generated by fusing scFv to the C-termini of constant heavy region domain 3 (CH3-scFv) and/or to the hinge region (scFv-hinge-Fc) of an antibody with a different specificity. Trivalent entities can also be made which have disulfide stabilized variable domains (without peptide linker) fused to the C-terminus of CH3 domains of IgGs.

### Fc-Containing Multispecific Molecules

In some embodiments, the multispecific molecules disclosed herein includes an immunoglobulin constant region (e.g., an Fc region). Exemplary Fc regions can be chosen from the heavy chain constant regions of IgG1, IgG2, IgG3 or IgG4; more particularly, the heavy chain constant region of human IgG1, IgG2, IgG3, or IgG4.

In some embodiments, the immunoglobulin chain constant region (e.g., the Fc region) is altered, e.g., mutated, to increase or decrease one or more of: Fc receptor binding, antibody glycosylation, the number of cysteine residues, effector cell function, or complement function.

In other embodiments, an interface of a first and second immunoglobulin chain constant regions (e.g., a first and a second Fc region) is altered, e.g., mutated, to increase or decrease dimerization, e.g., relative to a non-engineered interface, e.g., a naturally-occurring interface. For example, dimerization of the immunoglobulin chain constant region (e.g., the Fc region) can be enhanced by providing an Fc interface of a first and a second Fc region with one or more of: a paired protuberance-cavity (“knob-in-a hole”), an electrostatic interaction, or a strand-exchange, such that a greater ratio of heteromultimer to homomultimer forms, e.g., relative to a non-engineered interface.

In some embodiments, the multispecific molecules include a paired amino acid substitution at a position chosen from one or more of 347, 349, 350, 351, 366, 368, 370, 392, 394, 395, 397, 398, 399, 405, 407, or 409, e.g., of the Fc region of human IgG1 For example, the immunoglobulin chain constant region (e.g., Fc region) can include a paired an amino acid substitution chosen from: T366S, L368A, or Y407V (e.g., corresponding to a cavity or hole), and T366W (e.g., corresponding to a protuberance or knob).

In other embodiments, the multifunctional molecule includes a half-life extender, e.g., a human serum albumin or an antibody molecule to human serum albumin.

### Heterodimerized Antibody Molecules & Methods of Making

Various methods of producing multispecific antibodies have been disclosed to address the problem of incorrect heavy chain pairing. Exemplary methods are described below. Exemplary multispecific antibody formats and methods of making said multispecific antibodies are also disclosed in e.g., Speiss et al. Molecular Immunology 67 (2015) 95-106; and Klein et al mAbs 4:6, 653-663; November/December 2012; the entire contents of each of which are incorporated by reference herein.

Heterodimerized bispecific antibodies are based on the natural IgG structure, wherein the two binding arms recognize different antigens. IgG derived formats that enable defined monovalent (and simultaneous) antigen binding are generated by forced heavy chain heterodimerization, combined with technologies that minimize light chain mispairing (e.g., common light chain). Forced heavy chain heterodimerization can be obtained using, e.g., knob-in-hole OR strand exchange engineered domains (SEED).

### Knob-In-Hole

Knob-in-Hole as described in U.S. Pat. Nos. 5,731,116, 7,476,724 and Ridgway, J. et al. (1996) Prot. Engineering 9(7): 617-621, broadly involves: (1) mutating the CH3 domain of one or both antibodies to promote heterodimerization; and (2) combining the mutated antibodies under conditions that promote heterodimerization. “Knobs” or “protuberances” are typically created by replacing a small amino acid in a parental antibody with a larger amino acid (e.g., T366Y or T366W); “Holes” or “cavities” are created by replacing a larger residue in a parental antibody with a smaller amino acid (e.g., Y407T, T366S, L368A and/or Y407V).

For bispecific antibodies including an Fc domain, introduction of specific mutations into the constant region of the heavy chains to promote the correct heterodimerization of the Fc portion can be utilized. Several such techniques are reviewed in Klein et al. (mAbs (2012) 4:6, 1-11), the contents of which are incorporated herein by reference in their entirety. These techniques include the “knobs-into-holes” (KiH) approach which involves the introduction of a bulky residue into one of the CH3 domains of one of the antibody heavy chains. This bulky residue fits into a complementary “hole” in the other CH3 domain of the paired heavy chain so as to promote correct pairing of heavy chains (see e.g., U.S. Pat. No. 7,642,228).

Exemplary KiH mutations include S354C, T366W in the “knob” heavy chain and Y349C, T366S, L368A, Y407V in the “hole” heavy chain. Other exemplary KiH mutations are provided in Table 1, with additional optional stabilizing Fc cysteine mutations.

TABLE 1
Exemplary Fc KiH mutations and optional Cysteine mutations

| Position | Knob Mutation | Hole Mutation |
|---|---|---|
| T366 | T366W | T366S |
| L368 | — | L368A |
| Y407 | — | Y407V |

Additional Cysteine Mutations to form a stabilizing disulfide bridge

| Position | Knob CH3 | Hole CH3 |
|---|---|---|
| S354 | S354C | — |
| Y349 | — | Y349C |

Other Fe mutations are provided by Igawa and Tsunoda who identified 3 negatively charged residues in the CH3 domain of one chain that pair with three positively charged residues in the CH3 domain of the other chain. These specific charged residue pairs are: E356-K439, E357-K370, D399-K409 and vice versa. By introducing at least two of the following three mutations in chain A: E356K, E357K and D399K, as well as K370E, K409D, K439E in chain B, alone or in combination with newly identified disulfide bridges, they were able to favor very efficient heterodimerization while suppressing homodimerization at the same time (Martens T et al. A novel one-armed antic-Met antibody inhibits glioblastoma growth in vivo. Clin Cancer Res 2006; 12:6144-52; PMID: 17062691). Xencor defined 41 variant pairs based on combining structural calculations and sequence information that were subsequently screened for maximal heterodimerization, defining the combination of S364H, F405A (HA) on chain A and Y349T, T394F on chain B (TF) (Moore G L et al. A novel bispecific antibody format enables simultaneous bivalent and monovalent co-engagement of distinct target antigens. MAbs 2011; 3:546-57; PMID: 22123055).

Other exemplary Fc mutations to promote heterodimerization of multispecific antibodies include those described in the following references, the contents of each of which is incorporated by reference herein, WO2016071377A1, US20140079689A1, US20160194389A1, US20160257763, WO2016071376A2, WO2015107026A1, WO2015107025A1, WO2015107015A1, US20150353636A1, US20140199294A1, U.S. Pat. No. 7,750,128B2, US20160229915A1, US20150344570A1, U.S. Pat. No. 8,003,774A1, US20150337049A1, US20150175707A1, US20140242075A1, US20130195849A1, US20120149876A1, US20140200331A1, U.S. Pat. No. 9,309,311B2, U.S. Pat. No. 8,586,713, US20140037621A1, US20130178605A1, US20140363426A1, US20140051835A1 and US20110054151A1.

Stabilizing cysteine mutations have also been used in combination with KiH and other Fc heterodimerization promoting variants, see e.g., U.S. Pat. No. 7,183,076. Other exemplary cysteine modifications include, e.g., those disclosed in US20140348839A1, U.S. Pat. No. 7,855,275B2, and U.S. Pat. No. 9,000,130B2.

### Strand Exchange Engineered Domains (SEED)

Heterodimeric Fc platform that support the design of bispecific and asymmetric fusion proteins by devising strand-exchange engineered domain (SEED) C(H)3 heterodimers are known. These derivatives of human IgG and IgA C(H)3 domains create complementary human SEED C(H)3 heterodimers that are composed of alternating segments of human IgA and IgG C(H)3 sequences. The resulting pair of SEED C(H)3 domains preferentially associates to form heterodimers when expressed in mammalian cells. SEEDbody (Sb) fusion proteins consist of [IgG1 hinge]-C(H)2-[SEED C(H)3], that may be genetically linked to one or more fusion partners (see e.g., Davis J H et al. SEEDbodies: fusion proteins based on strand exchange engineered domain (SEED) CH3 heterodimers in an Fc analogue platform for asymmetric binders or immunofusions and bispecific antibodies. Protein Eng Des Sel 2010; 23:195-202; PMID:20299542 and U.S. Pat. No. 8,871,912. The contents of each of which are incorporated by reference herein).

### Duobody

“Duobody” technology to produce bispecific antibodies with correct heavy chain pairing are known. The DuoBody technology involves three basic steps to generate stable bispecific human IgG1 antibodies in a post-production exchange reaction. In a first step, two IgG1s, each containing single matched mutations in the third constant (CH3) domain, are produced separately using standard mammalian recombinant cell lines. Subsequently, these IgG1 antibodies are purified according to standard processes for recovery and purification. After production and purification (post-production), the two antibodies are recombined under tailored laboratory conditions resulting in a bispecific antibody product with a very high yield (typically >95%) (see e.g., Labrijn et al, PNAS 2013; 110(13):5145-5150 and Labrijn et al. Nature Protocols 2014; 9(10):2450-63, the contents of each of which are incorporated by reference herein).

### Electrostatic Interactions

Methods of making multispecific antibodies using CH3 amino acid changes with charged amino acids such that homodimer formation is electrostatically unfavorable are disclosed. EP1870459 and WO 2009089004 describe other strategies for favoring heterodimer formation upon co-expression of different antibody domains in a host cell. In these methods, one or more residues that make up the heavy chain constant domain 3 (CH3), CH3-CH3 interfaces in both CH3 domains are replaced with a charged amino acid such that homodimer formation is electrostatically unfavorable and heterodimerization is electrostatically favorable. Additional methods of making multispecific molecules using electrostatic interactions are described in the following references, the contents of each of which is incorporated by reference herein, include US20100015133, U.S. Pat. No. 8,592,562B2, U.S. Pat. No. 9,200,060B2, US20140154254A1, and U.S. Pat. No. 9,358,286A1.

### Common Light Chain

Light chain mispairing needs to be avoided to generate homogenous preparations of bispecific IgGs. One way to achieve this is through the use of the common light chain principle, i.e. combining two binders that share one light chain but still have separate specificities. An exemplary method of enhancing the formation of a desired bispecific antibody from a mixture of monomers is by providing a common variable light chain to interact with each of the heteromeric variable heavy chain regions of the bispecific antibody. Compositions and methods of producing bispecific antibodies with a common light chain as disclosed in, e.g., U.S. Pat. No. 7,183,076B2, US20110177073A1, EP2847231A1, WO2016079081A1, and EP3055329A1, the contents of each of which is incorporated by reference herein.

### CrossMab

Another option to reduce light chain mispairing is the CrossMab technology which avoids non-specific L chain mispairing by exchanging CH1 and CL domains in the Fab of one half of the bispecific antibody. Such crossover variants retain binding specificity and affinity, but make the two arms so different that L chain mispairing is prevented. The CrossMab technology (as reviewed in Klein et al. Supra) involves domain swapping between heavy and light chains so as to promote the formation of the correct pairings. Briefly, to construct a bispecific IgG-like CrossMab antibody that could bind to two antigens by using two distinct light chain-heavy chain pairs, a two-step modification process is applied. First, a dimerization interface is engineered into the C-terminus of each heavy chain using a heterodimerization approach, e.g., Knob-into-hole (KiH) technology, to ensure that only a heterodimer of two distinct heavy chains from one antibody (e.g., Antibody A) and a second antibody (e.g., Antibody B) is efficiently formed. Next, the constant heavy 1 (CH1) and constant light (CL) domains of one antibody are exchanged (Antibody A), keeping the variable heavy (VH) and variable light (VL) domains consistent. The exchange of the CH1 and CL domains ensured that the modified antibody (Antibody A) light chain would only efficiently dimerize with the modified antibody (antibody A) heavy chain, while the unmodified antibody (Antibody B) light chain would only efficiently dimerize with the unmodified antibody (Antibody B) heavy chain; and thus only the desired bispecific CrossMab would be efficiently formed (see e.g., Cain, C. SciBX 4(28); doi:10.1038/scibx.2011.783, the contents of which are incorporated by reference herein).

### Common Heavy Chain

An exemplary method of enhancing the formation of a desired bispecific antibody from a mixture of monomers is by providing a common variable heavy chain to interact with each of the heteromeric variable light chain regions of the bispecific antibody. Compositions and methods of producing bispecific antibodies with a common heavy chain are disclosed in, e.g., US20120184716, US20130317200, and US20160264685A1, the contents of each of which is incorporated by reference herein.

### Amino Acid Modifications

Alternative compositions and methods of producing multispecific antibodies with correct light chain pairing include various amino acid modifications. For example, Zymeworks describes heterodimers with one or more amino acid modifications in the CH1 and/or CL domains, one or more amino acid modifications in the VH and/or VL domains, or a combination thereof, which are part of the interface between the light chain and heavy chain and create preferential pairing between each heavy chain and a desired light chain such that when the two heavy chains and two light chains of the heterodimer pair are co-expressed in a cell, the heavy chain of the first heterodimer preferentially pairs with one of the light chains rather than the other (see e.g., WO2015181805). Other exemplary methods are described in WO2016026943 (Argen-X), US20150211001, US20140072581A1, US20160039947A1, and US20150368352.

### Lambda Kappa Formats

Multispecific molecules (e.g., multispecific antibody molecules) that include the lambda light chain polypeptide and a kappa light chain polypeptides, can be used to allow for heterodimerization. Methods for generating bispecific antibody molecules comprising the lambda light chain polypeptide and a kappa light chain polypeptides are disclosed in PCT Publication No. WO2018057955 (corresponding to PCT/US17/53053, filed on Sep. 22, 2017), incorporated herein by reference in its entirety.

In some embodiments, the multispecific molecules includes a multispecific antibody molecule, e.g., an antibody molecule comprising two binding specificities, e.g., a bispecific antibody molecule. The multispecific antibody molecule includes:
a lambda light chain polypeptide 1 (LLCP1) specific for a first epitope;
   a heavy chain polypeptide 1 (HCP1) specific for the first epitope;
   a kappa light chain polypeptide 2 (KLCP2) specific for a second epitope; and
   a heavy chain polypeptide 2 (HCP2) specific for the second epitope.

“Lambda light chain polypeptide 1 (LLCP1)”, as that term is used herein, refers to a polypeptide comprising sufficient light chain (LC) sequence, such that when combined with a cognate heavy chain variable region, can mediate specific binding to its epitope and complex with an HCP1. In an embodiment it comprises all or a fragment of a CH1 region. In an embodiment, an LLCP1 comprises LC-CDR1, LC-CDR2, LC-CDR3, FR1, FR2, FR3, FR4, and CH1, or sufficient sequence therefrom to mediate specific binding of its epitope and complex with an HCP1. LLCP1, together with its HCP1, provide specificity for a first epitope (while KLCP2, together with its HCP2, provide specificity for a second epitope). As described elsewhere herein, LLCP1 has a higher affinity for HCP1 than for HCP2.

“Kappa light chain polypeptide 2 (KLCP2)”, as that term is used herein, refers to a polypeptide comprising sufficient light chain (LC) sequence, such that when combined with a cognate heavy chain variable region, can mediate specific binding to its epitope and complex with an HCP2. In an embodiments it comprises all or a fragment of a CH1 region. In an embodiment, a KLCP2 comprises LC-CDR1, LC-CDR2, LC-CDR3, FR1, FR2, FR3, FR4, and CH1, or sufficient sequence therefrom to mediate specific binding of its epitope and complex with an HCP2. KLCP2, together with its HCP2, provide specificity for a second epitope (while LLCP1, together with its HCP1, provide specificity for a first epitope).

“Heavy chain polypeptide 1 (HCP1)”, as that term is used herein, refers to a polypeptide comprising sufficient heavy chain (HC) sequence, e.g., HC variable region sequence, such that when combined with a cognate LLCP1, can mediate specific binding to its epitope and complex with an HCP1. In an embodiments it comprises all or a fragment of a CH1 region. In an embodiment, it comprises all or a fragment of a CH2 and/or CH3 region. In an embodiment an HCP1 comprises HC-CDR1, HC-CDR2, HC-CDR3, FR1, FR2, FR3, FR4, CH1, CH2, and CH3, or sufficient sequence therefrom to: (i) mediate specific binding of its epitope and complex with an LLCP1, (ii) to complex preferentially, as described herein to LLCP1 as opposed to KLCP2; and (iii) to complex preferentially, as described herein, to an HCP2, as opposed to another molecule of HCP1. HCP1, together with its LLCP1, provide specificity for a first epitope (while KLCP2, together with its HCP2, provide specificity for a second epitope).

“Heavy chain polypeptide 2 (HCP2)”, as that term is used herein, refers to a polypeptide comprising sufficient heavy chain (HC) sequence, e.g., HC variable region sequence, such that when combined with a cognate LLCP1, can mediate specific binding to its epitope and complex with an HCP1. In an embodiments it comprises all or a fragment of a CH1 region. In an embodiments it comprises all or a fragment of a CH2 and/or CH3 region. In an embodiment an HCP1 comprises HC-CDR1, HC-CDR2, HC-CDR3, FR1, FR2, FR3, FR4, CH1, CH2, and CH3, or sufficient sequence therefrom to: (i) mediate specific binding of its epitope and complex with an KLCP2, (ii) to complex preferentially, as described herein to KLCP2 as opposed to LLCP1; and (iii) to complex preferentially, as described herein, to an HCP1, as opposed to another molecule of HCP2. HCP2, together with its KLCP2, provide specificity for a second epitope (while LLCP1, together with its HCP1, provide specificity for a first epitope).

In some embodiments of the multispecific antibody molecule disclosed herein:
LLCP1 has a higher affinity for HCP1 than for HCP2; and/or
KLCP2 has a higher affinity for HCP2 than for HCP1.

In some embodiments, the affinity of LLCP1 for HCP1 is sufficiently greater than its affinity for HCP2, such that under preselected conditions, e.g., in aqueous buffer, e.g., at pH 7, in saline, e.g., at pH 7, or under physiological conditions, at least 75, 80, 90, 95, 98, 99, 99.5, or 99.9% of the multispecific antibody molecule molecules have a LLCP1 complexed, or interfaced with, a HCP1.

In some embodiments of the multispecific antibody molecule disclosed herein: the HCP1 has a greater affinity for HCP2, than for a second molecule of HCP1; and/or
the HCP2 has a greater affinity for HCP1, than for a second molecule of HCP2.

In some embodiments, the affinity of HCP1 for HCP2 is sufficiently greater than its affinity for a second molecule of HCP1, such that under preselected conditions, e.g., in aqueous buffer, e.g., at pH 7, in saline, e.g., at pH 7, or under physiological conditions, at least 75%, 80, 90, 95, 98, 99 99.5 or 99.9% of the multispecific antibody molecule molecules have a HCP1 complexed, or interfaced with, a HCP2.

In another aspect, disclosed herein is a method for making, or producing, a multispecific antibody molecule. The method includes:
(i) providing a first heavy chain polypeptide (e.g., a heavy chain polypeptide comprising one, two, three or all of a first heavy chain variable region (first VH), a first CH1, a first heavy chain constant region (e.g., a first CH2, a first CH3, or both));
(ii) providing a second heavy chain polypeptide (e.g., a heavy chain polypeptide comprising one, two, three or all of a second heavy chain variable region (second VH), a second CH1, a second heavy chain constant region (e.g., a second CH2, a second CH3, or both));
(iii) providing a lambda chain polypeptide (e.g., a lambda light variable region (VL□), a lambda light constant chain (VL□), or both) that preferentially associates with the first heavy chain polypeptide (e.g., the first VH); and
(iv) providing a kappa chain polypeptide (e.g., a lambda light variable region (VL□), a lambda light constant chain (VL□), or both) that preferentially associates with the second heavy chain polypeptide (e.g., the second VH), under conditions where (i)-(iv) associate.

In some embodiments, the first and second heavy chain polypeptides form an Fc interface that enhances heterodimerization.

In some embodiments, (i)-(iv) (e.g., nucleic acid encoding (i)-(iv)) are introduced in a single cell, e.g., a single mammalian cell, e.g., a CHO cell. In some embodiments, (i)-(iv) are expressed in the cell.

In some embodiments, (i)-(iv) (e.g., nucleic acid encoding (i)-(iv)) are introduced in different cells, e.g., different mammalian cells, e.g., two or more CHO cell. In some embodiments, (i)-(iv) are expressed in the cells.

In one embodiment, the method further comprises purifying a cell-expressed antibody molecule, e.g., using a lambda- and/or kappa-specific purification, e.g., affinity chromatography.

In some embodiments, the method further comprises evaluating the cell-expressed multispecific antibody molecule. For example, the purified cell-expressed multispecific antibody molecule can be analyzed by techniques known in the art, include mass spectrometry. In one embodiment, the purified cell-expressed antibody molecule is cleaved, e.g., digested with papain to yield the Fab moieties and evaluated using mass spectrometry.

In some embodiments, the method produces correctly paired kappa/lambda multispecific, e.g., bispecific, antibody molecules in a high yield, e.g., at least 75%, 80, 90, 95, 98, 99 99.5 or 99.9%.

In other embodiments, the multispecific, e.g., a bispecific, antibody molecule that includes:
(i) a first heavy chain polypeptide (HCP1) (e.g., a heavy chain polypeptide comprising one, two, three or all of a first heavy chain variable region (first VH), a first CH1, a first heavy chain constant region (e.g., a first CH2, a first CH3, or both)), e.g., wherein the HCP1 binds to a first epitope;
(ii) a second heavy chain polypeptide (HCP2) (e.g., a heavy chain polypeptide comprising one, two, three or all of a second heavy chain variable region (second VH), a second CH1, a second heavy chain constant region (e.g., a second CH2, a second CH3, or both)), e.g., wherein the HCP2 binds to a second epitope;
(iii) a lambda light chain polypeptide (LLCP1) (e.g., a lambda light variable region (VL1), a lambda light constant chain (VL1), or both) that preferentially associates with the first heavy chain polypeptide (e.g., the first VH), e.g., wherein the LLCP1 binds to a first epitope; and
(iv) a kappa light chain polypeptide (KLCP2) (e.g., a lambda light variable region (VLk), a lambda light constant chain (VLk), or both) that preferentially associates with the second heavy chain polypeptide (e.g., the second VH), e.g., wherein the KLCP2 binds to a second epitope.

In some embodiments, the first and second heavy chain polypeptides form an Fc interface that enhances heterodimerization. In some embodiments, the multispecific antibody molecule has a first binding specificity that includes a hybrid VL1-CL1 heterodimerized to a first heavy chain variable region connected to the Fc constant, CH2-CH3 domain (having a knob modification) and a second binding specificity that includes a hybrid VLk-CLk heterodimerized to a second heavy chain variable region connected to the Fc constant, CH2-CH3 domain (having a hole modification).

### Calreticulin-Targeting Antigen Binding Domains

The present disclosure provides, inter alia, multispecific (e.g., bi-, tri-, tetra-specific) or multifunctional molecules, that include, e.g., are engineered to contain, one or more antigen binding domains that bind to calreticulin, e.g., a wild-type calreticulin protein or a calreticulin mutant protein. In some embodiments, the multifunctional molecule binds to a wild-type calreticulin protein and a calreticulin mutant protein with similar affinity. In some embodiments, the multifunctional molecule preferentially binds to a calreticulin mutant protein over a wild type calreticulin protein.

An exemplary wild type human calreticulin is shown as SEQ ID NO: 6285.

(SEQ ID NO: 6285)
EPAVYFKEQFLDGDGWTSRWIESKHKSDFGKFVLSSGKFYGDEEKDKGLQ
TSQDARFYALSASFEPFSNKGQTLVVQFTVKHEQNIDCGGGYVKLFPNSL
DQTDMHGDSEYNIMFGPDICGPGTKKVHVIFNYKGKNVLINKDIRCKDDE
FTHLYTLIVRPDNTYEVKIDNSQVESGSLEDDWDFLPPKKIKDPDASKPE
DWDERAKIDDPTDSKPEDWDKPEHIPDPDAKKPEDWDEEMDGEWEPPVIQ
NPEYKGEWKPRQIDNPDYKGTWIHPEIDNPEYSPDPSIYAYDNFGVLGLD
LWQVKSGTIFDNFLITNDEAYAEEFGNETWGVTKAAEKQMKDKQDEEQRL
KEEEEDKKRKEEEEAEDKEDDEDKDEDEEDEEDKEEDEEEDVPGQAKDEL

An additional exemplary wild type human calreticulin is shown as SEQ ID NO: 1001:

(SEQ ID NO: 1001)
MLLSVPLLLGLLGLAVAHHHHHHHHGGGGSEPAVYFKEQFLDGDGWTSRW
IESKHKSDFGKFVLSSGKFYGDEEKDKGLQTSQDARFYALSASFEPFSNK
GQTLVVQFTVKHEQNIDCGGGYVKLFPNSLDQTDMHGDSEYNIMFGPDIC
GPGTKKVHVIFNYKGKNVLINKDIRCKDDEFTHLYTLIVRPDNTYEVKID
NSQVESGSLEDDWDFLPPKKIKDPDASKPEDWDERAKIDDPTDSKPEDWD
KPEHIPDPDAKKPEDWDEEMDGEWEPPVIQNPEYKGEWKPRQIDNPDYKG
TWIHPEIDNPEYSPDPSIYAYDNFGVLGLDLWQVKSGTIFDNFLITNDEA
YAEEFGNETWGVTKAAEKQMKDKQDEEQRLKEEEEDKKRKEEEEAEDKED
DEDKDEDEEDEEDKEEDEEEDVPGQA

Calreticulin mutant proteins have been identified and found to be associated with myeloid cancers, e.g., see Nangalia et al., N Engl J Med. 2013 Dec. 19; 369(25):2391-2405, Klampfl et al., N Engl J Med. 2013 Dec. 19; 369(25):2379-90, and US20170269092, herein incorporated by reference in their entirety. Mutant calreticulin has a frameshift in exon 9 of the coding sequence of wild type calreticulin, resulting in the replacement of the C-terminal negatively charged amino acids of wild type calreticulin by a predominantly positively charged polypeptide. Table 2 discloses full-length amino acid sequences of 38 calreticulin mutant proteins. Table 3 discloses the C-terminal amino acid sequences of the 36 calreticulin mutant proteins. All 38 calreticulin mutant proteins comprise the amino acid sequence of RRKMSPARPRTSCREACLQGWTEA (SEQ ID NO: 6286).

The predominant mutations of calreticulin are Type 1 and Type 2 mutations (see Tables 2 and 3). Type 1 mutation is a 52-bp deletion (c.1092_1143del) whereas Type 2 mutation is a 5-bp insertion (c.1154_1155insTTGTC).

TABLE 2
Full-length amino acid sequences of calreticulin mutants

| SEQ | | |
|---|---|---|
| ID NO | Type | Full length sequences of insertion/deletion frameshift mutations of calreticulin |
| SEQ | Type 1 | EPAVYFKEQFLDGDGWTSRWIESKHKSDFGKFVLSSGKFYGDEEKDKGL |
| ID NO: | | QTSQDARFYALSASFEPFSNKGQTLVVQFTVKHEQNIDCGGGYVKLFPNS |
| 6313 | | LDQTDMHGDSEYNIMFGPDICGPGTKKVHVIFNYKGKNVLINKDIRCKD |
| | | DEFTHLYTLIVRPDNTYEVKIDNSQVESGSLEDDWDFLPPKKIKDPDASK |
| | | PEDWDERAKIDDPTDSKPEDWDKPEHIPDPDAKKPEDWDEEMDGEWEP |
| | | PVIQNPEYKGEWKPRQIDNPDYKGTWIHPEIDNPEYSPDPSIYAYDNFGV |
| | | LGLDLWQVKSGTIFDNFLITNDEAYAEEFGNETWGVTKAAEKQMKDKQ |
| | | DEEQRTRRMMRTKMRMRRMRRTRRKMRRKMSPARPRTSCREACLQG |
| | | WTEA |
| SEQ | Type 2 | EPAVYFKEQFLDGDGWTSRWIESKHKSDFGKFVLSSGKFYGDEEKDKGL |
| ID NO: | | QTSQDARFYALSASFEPFSNKGQTLVVQFTVKHEQNIDCGGGYVKLFPNS |
| 6314 | | LDQTDMHGDSEYNIMFGPDICGPGTKKVHVIFNYKGKNVLINKDIRCKD |
| | | DEFTHLYTLIVRPDNTYEVKIDNSQVESGSLEDDWDFLPPKKIKDPDASK |
| | | PEDWDERAKIDDPTDSKPEDWDKPEHIPDPDAKKPEDWDEEMDGEWEP |
| | | PVIQNPEYKGEWKPRQIDNPDYKGTWIHPEIDNPEYSPDPSIYAYDNFGV |
| | | LGLDLWQVKSGTIFDNFLITNDEAYAEEFGNETWGVTKAAEKQMKDKQ |
| | | DEEQRLKEEEEDKKRKEEEEAEDNCRRMMRTKMRMRRMRRTRRKMRR |
| | | KMSPARPRTSCREACLQGWTEA |
| SEQ | Type 3 | EPAVYFKEQFLDGDGWTSRWIESKHKSDFGKFVLSSGKFYGDEEKDKGL |
| ID NO: | | QTSQDARFYALSASFEPFSNKGQTLVVQFTVKHEQNIDCGGGYVKLFPNS |
| 6315 | | LDQTDMHGDSEYNIMFGPDICGPGTKKVHVIFNYKGKNVLINKDIRCKD |
| | | DEFTHLYTLIVRPDNTYEVKIDNSQVESGSLEDDWDFLPPKKIKDPDASK |
| | | PEDWDERAKIDDPTDSKPEDWDKPEHIPDPDAKKPEDWDEEMDGEWEP |
| | | PVIQNPEYKGEWKPRQIDNPDYKGTWIHPEIDNPEYSPDPSIYAYDNFGV |
| | | LGLDLWQVKSGTIFDNFLITNDEAYAEEFGNETWGVTKAAEKQMKDKQ |
| | | DEEQRQRTRRMMRTKMRMRRMRRTRRKMRRKMSPARPRTSCREACLQ |
| | | GWTEA |
| SEQ | Type 4 | EPAVYFKEQFLDGDGWTSRWIESKHKSDFGKFVLSSGKFYGDEEKDKGL |
| ID NO: | | QTSQDARFYALSASFEPFSNKGQTLVVQFTVKHEQNIDCGGGYVKLFPNS |
| 6316 | | LDQTDMHGDSEYNIMFGPDICGPGTKKVHVIFNYKGKNVLINKDIRCKD |
| | | DEFTHLYTLIVRPDNTYEVKIDNSQVESGSLEDDWDFLPPKKIKDPDASK |
| | | PEDWDERAKIDDPTDSKPEDWDKPEHIPDPDAKKPEDWDEEMDGEWEP |
| | | PVIQNPEYKGEWKPRQIDNPDYKGTWIHPEIDNPEYSPDPSIYAYDNFGV |
| | | LGLDLWQVKSGTIFDNFLITNDEAYAEEFGNETWGVTKAAEKQMKDKQ |
| | | DEEQRLRRRQRTRRMMRTKMRMRRMRRTRRKMRRKMSPARPRTSCRE |
| | | ACLQGWTEA |
| SEQ | Type 5 | EPAVYFKEQFLDGDGWTSRWIESKHKSDFGKFVLSSGKFYGDEEKDKGL |
| ID NO: | | QTSQDARFYALSASFEPFSNKGQTLVVQFTVKHEQNIDCGGGYVKLFPNS |
| 6317 | | LDQTDMHGDSEYNIMFGPDICGPGTKKVHVIFNYKGKNVLINKDIRCKD |
| | | DEFTHLYTLIVRPDNTYEVKIDNSQVESGSLEDDWDFLPPKKIKDPDASK |
| | | PEDWDERAKIDDPTDSKPEDWDKPEHIPDPDAKKPEDWDEEMDGEWEP |
| | | PVIQNPEYKGEWKPRQIDNPDYKGTWIHPEIDNPEYSPDPSIYAYDNFGV |
| | | LGLDLWQVKSGTIFDNFLITNDEAYAEEFGNETWGVTKAAEKQMKDKQ |
| | | DEGQRTRRMMRTKMRMRRMRRTRRKMRRKMSPARPRTSCREACLQG |
| | | WTEA |
| SEQ | Type 6 | EPAVYFKEQFLDGDGWTSRWIESKHKSDFGKFVLSSGKFYGDEEKDKGL |
| ID NO: | | QTSQDARFYALSASFEPFSNKGQTLVVQFTVKHEQNIDCGGGYVKLFPNS |
| 6318 | | LDQTDMHGDSEYNIMFGPDICGPGTKKVHVIFNYKGKNVLINKDIRCKD |
| | | DEFTHLYTLIVRPDNTYEVKIDNSQVESGSLEDDWDFLPPKKIKDPDASK |
| | | PEDWDERAKIDDPTDSKPEDWDKPEHIPDPDAKKPEDWDEEMDGEWEP |
| | | PVIQNPEYKGEWKPRQIDNPDYKGTWIHPEIDNPEYSPDPSIYAYDNFGV |
| | | LGLDLWQVKSGTIFDNFLITNDEAYAEEFGNETWGVTKAAEKQMKDKQ |
| | | DEERRQRTRRMMRTKMRMRRMRRTRRKMRRKMSPARPRTSCREACLQ |
| | | GWTEA |
| SEQ | Type 7 | EPAVYFKEQFLDGDGWTSRWIESKHKSDFGKFVLSSGKFYGDEEKDKGL |
| ID NO: | | QTSQDARFYALSASFEPFSNKGQTLVVQFTVKHEQNIDCGGGYVKLFPNS |
| 6319 | | LDQTDMHGDSEYNIMFGPDICGPGTKKVHVIFNYKGKNVLINKDIRCKD |
| | | DEFTHLYTLIVRPDNTYEVKIDNSQVESGSLEDDWDFLPPKKIKDPDASK |
| | | PEDWDERAKIDDPTDSKPEDWDKPEHIPDPDAKKPEDWDEEMDGEWEP |
| | | PVIQNPEYKGEWKPRQIDNPDYKGTWIHPEIDNPEYSPDPSIYAYDNFGV |
| | | LGLDLWQVKSGTIFDNFLITNDEAYAEEFGNETWGVTKAAEKQMKDKQ |
| | | DEEQRLRRMMRTKMRMRRMRRTRRKMRRKMSPARPRTSCREACLQG |
| | | WTEA |
| SEQ | Type 8 | EPAVYFKEQFLDGDGWTSRWIESKHKSDFGKFVLSSGKFYGDEEKDKGL |
| ID NO: | | QTSQDARFYALSASFEPFSNKGQTLVVQFTVKHEQNIDCGGGYVKLFPNS |
| 6320 | | LDQTDMHGDSEYNIMFGPDICGPGTKKVHVIFNYKGKNVLINKDIRCKD |
| | | DEFTHLYTLIVRPDNTYEVKIDNSQVESGSLEDDWDFLPPKKIKDPDASK |
| | | PEDWDERAKIDDPTDSKPEDWDKPEHIPDPDAKKPEDWDEEMDGEWEP |
| | | PVIQNPEYKGEWKPRQIDNPDYKGTWIHPEIDNPEYSPDPSIYAYDNFGV |
| | | LGLDLWQVKSGTIFDNFLITNDEAYAEEFGNETWGVTKAAEKQMKDKQ |
| | | DEEQRLKRRQRTRRMMRTKMRMRRMRRTRRKMRRKMSPARPRTSCRE |
| | | ACLQGWTEA |
| SEQ | Type 9 | EPAVYFKEQFLDGDGWTSRWIESKHKSDFGKFVLSSGKFYGDEEKDKGL |
| ID NO: | | QTSQDARFYALSASFEPFSNKGQTLVVQFTVKHEQNIDCGGGYVKLFPNS |
| 6321 | | LDQTDMHGDSEYNIMFGPDICGPGTKKVHVIFNYKGKNVLINKDIRCKD |
| | | DEFTHLYTLIVRPDNTYEVKIDNSQVESGSLEDDWDFLPPKKIKDPDASK |
| | | PEDWDERAKIDDPTDSKPEDWDKPEHIPDPDAKKPEDWDEEMDGEWEP |
| | | PVIQNPEYKGEWKPRQIDNPDYKGTWIHPEIDNPEYSPDPSIYAYDNFGV |
| | | LGLDLWQVKSGTIFDNFLITNDEAYAEEFGNETWGVTKAAEKQMKDKQ |
| | | DEEQRLKEEEEDKKRKEEERQRTRRMMRTKMRMRRMRRTRRKMRRK |
| | | MSPARPRTSCREACLQGWTEA |
| SEQ | Type | EPAVYFKEQFLDGDGWTSRWIESKHKSDFGKFVLSSGKFYGDEEKDKGL |
| ID NO: | 10 | QTSQDARFYALSASFEPFSNKGQTLVVQFTVKHEQNIDCGGGYVKLFPNS |
| 6322 | | LDQTDMHGDSEYNIMFGPDICGPGTKKVHVIFNYKGKNVLINKDIRCKD |
| | | DEFTHLYTLIVRPDNTYEVKIDNSQVESGSLEDDWDFLPPKKIKDPDASK |
| | | PEDWDERAKIDDPTDSKPEDWDKPEHIPDPDAKKPEDWDEEMDGEWEP |
| | | PVIQNPEYKGEWKPRQIDNPDYKGTWIHPEIDNPEYSPDPSIYAYDNFGV |
| | | LGLDLWQVKSGTIFDNFLITNDEAYAEEFGNETWGVTKAAEKQMKDKQ |
| | | DEEQRLKEEEEDKKRKEEEEAEDMCRRMMRTKMRMRRMRRTRRKMRR |
| | | KMSPARPRTSCREACLQGWTEA |
| SEQ | Type | EPAVYFKEQFLDGDGWTSRWIESKHKSDFGKFVLSSGKFYGDEEKDKGL |
| ID NO: | 11 | QTSQDARFYALSASFEPFSNKGQTLVVQFTVKHEQNIDCGGGYVKLFPNS |
| 6323 | | LDQTDMHGDSEYNIMFGPDICGPGTKKVHVIFNYKGKNVLINKDIRCKD |
| | | DEFTHLYTLIVRPDNTYEVKIDNSQVESGSLEDDWDFLPPKKIKDPDASK |
| | | PEDWDERAKIDDPTDSKPEDWDKPEHIPDPDAKKPEDWDEEMDGEWEP |
| | | PVIQNPEYKGEWKPRQIDNPDYKGTWIHPEIDNPEYSPDPSIYAYDNFGV |
| | | LGLDLWQVKSGTIFDNFLITNDEAYAEEFGNETWGVTKAAEKQMKDKQ |
| | | DEDQRQRTRRMMRTKMRMRRMRRTRRKMRRKMSPARPRTSCREACLQ |
| | | GWTEA |
| SEQ | Type | EPAVYFKEQFLDGDGWTSRWIESKHKSDFGKFVLSSGKFYGDEEKDKGL |
| ID NO: | 12 | QTSQDARFYALSASFEPFSNKGQTLVVQFTVKHEQNIDCGGGYVKLFPNS |
| 6324 | | LDQTDMHGDSEYNIMFGPDICGPGTKKVHVIFNYKGKNVLINKDIRCKD |
| | | DEFTHLYTLIVRPDNTYEVKIDNSQVESGSLEDDWDFLPPKKIKDPDASK |
| | | PEDWDERAKIDDPTDSKPEDWDKPEHIPDPDAKKPEDWDEEMDGEWEP |
| | | PVIQNPEYKGEWKPRQIDNPDYKGTWIHPEIDNPEYSPDPSIYAYDNFGV |
| | | LGLDLWQVKSGTIFDNFLITNDEAYAEEFGNETWGVTKAAEKQMKDKQ |
| | | DEEQRRRRRQRTRRMMRTKMRMRRMRRTRRKMRRKMSPARPRTSCRE |
| | | ACLQGWTEA |
| SEQ | Type | EPAVYFKEQFLDGDGWTSRWIESKHKSDFGKFVLSSGKFYGDEEKDKGL |
| ID NO: | 13 | QTSQDARFYALSASFEPFSNKGQTLVVQFTVKHEQNIDCGGGYVKLFPNS |
| 6325 | | LDQTDMHGDSEYNIMFGPDICGPGTKKVHVIFNYKGKNVLINKDIRCKD |
| | | DEFTHLYTLIVRPDNTYEVKIDNSQVESGSLEDDWDFLPPKKIKDPDASK |
| | | PEDWDERAKIDDPTDSKPEDWDKPEHIPDPDAKKPEDWDEEMDGEWEP |
| | | PVIQNPEYKGEWKPRQIDNPDYKGTWIHPEIDNPEYSPDPSIYAYDNFGV |
| | | LGLDLWQVKSGTIFDNFLITNDEAYAEEFGNETWGVTKAAEKQMKDKQ |
| | | DEEQRQRRRQRTRRMMRTKMRMRRMRRTRRKMRRKMSPARPRTSCRE |
| | | ACLQGWTEA |
| SEQ | Type | EPAVYFKEQFLDGDGWTSRWIESKHKSDFGKFVLSSGKFYGDEEKDKGL |
| ID NO: | 14 | QTSQDARFYALSASFEPFSNKGQTLVVQFTVKHEQNIDCGGGYVKLFPNS |
| 6316 | | LDQTDMHGDSEYNIMFGPDICGPGTKKVHVIFNYKGKNVLINKDIRCKD |
| | | DEFTHLYTLIVRPDNTYEVKIDNSQVESGSLEDDWDFLPPKKIKDPDASK |
| | | PEDWDERAKIDDPTDSKPEDWDKPEHIPDPDAKKPEDWDEEMDGEWEP |
| | | PVIQNPEYKGEWKPRQIDNPDYKGTWIHPEIDNPEYSPDPSIYAYDNFGV |
| | | LGLDLWQVKSGTIFDNFLITNDEAYAEEFGNETWGVTKAAEKQMKDKQ |
| | | DEEQRLRRRQRTRRMMRTKMRMRRMRRTRRKMRRKMSPARPRTSCRE |
| | | ACLQGWTEA |
| SEQ | Type | EPAVYFKEQFLDGDGWTSRWIESKHKSDFGKFVLSSGKFYGDEEKDKGL |
| ID NO: | 15 | QTSQDARFYALSASFEPFSNKGQTLVVQFTVKHEQNIDCGGGYVKLFPNS |
| 6326 | | LDQTDMHGDSEYNIMFGPDICGPGTKKVHVIFNYKGKNVLINKDIRCKD |
| | | DEFTHLYTLIVRPDNTYEVKIDNSQVESGSLEDDWDFLPPKKIKDPDASK |
| | | PEDWDERAKIDDPTDSKPEDWDKPEHIPDPDAKKPEDWDEEMDGEWEP |
| | | PVIQNPEYKGEWKPRQIDNPDYKGTWIHPEIDNPEYSPDPSIYAYDNFGV |
| | | LGLDLWQVKSGTIFDNFLITNDEAYAEEFGNETWGVTKAAEKQMKDKQ |
| | | DEEQRLRRRERTRRMMRTKMRMRRMRRTRRKMRRKMSPARPRTSCRE |
| | | ACLQGWTEA |
| SEQ | Type | EPAVYFKEQFLDGDGWTSRWIESKHKSDFGKFVLSSGKFYGDEEKDKGL |
| ID NO: | 16 | QTSQDARFYALSASFEPFSNKGQTLVVQFTVKHEQNIDCGGGYVKLFPNS |
| 6327 | | LDQTDMHGDSEYNIMFGPDICGPGTKKVHVIFNYKGKNVLINKDIRCKD |
| | | DEFTHLYTLIVRPDNTYEVKIDNSQVESGSLEDDWDFLPPKKIKDPDASK |
| | | PEDWDERAKIDDPTDSKPEDWDKPEHIPDPDAKKPEDWDEEMDGEWEP |
| | | PVIQNPEYKGEWKPRQIDNPDYKGTWIHPEIDNPEYSPDPSIYAYDNFGV |
| | | LGLDLWQVKSGTIFDNFLITNDEAYAEEFGNETWGVTKAAEKQMKDKQ |
| | | DEEQRLQRRQRTRRMMRTKMRMRRMRRTRRKMRRKMSPARPRTSCRE |
| | | ACLQGWTEA |
| SEQ | Type | EPAVYFKEQFLDGDGWTSRWIESKHKSDFGKFVLSSGKFYGDEEKDKGL |
| ID NO: | 17 | QTSQDARFYALSASFEPFSNKGQTLVVQFTVKHEQNIDCGGGYVKLFPNS |
| 6328 | | LDQTDMHGDSEYNIMFGPDICGPGTKKVHVIFNYKGKNVLINKDIRCKD |
| | | DEFTHLYTLIVRPDNTYEVKIDNSQVESGSLEDDWDFLPPKKIKDPDASK |
| | | PEDWDERAKIDDPTDSKPEDWDKPEHIPDPDAKKPEDWDEEMDGEWEP |
| | | PVIQNPEYKGEWKPRQIDNPDYKGTWIHPEIDNPEYSPDPSIYAYDNFGV |
| | | LGLDLWQVKSGTIFDNFLITNDEAYAEEFGNETWGVTKAAEKQMKDKQ |
| | | DEEQRLKRRQWTRRMMRTKMRMRRMRRTRRKMRRKMSPARPRTSCRE |
| | | ACLQGWTEA |
| SEQ | Type | EPAVYFKEQFLDGDGWTSRWIESKHKSDFGKFVLSSGKFYGDEEKDKGL |
| ID NO: | 18 | QTSQDARFYALSASFEPFSNKGQTLVVQFTVKHEQNIDCGGGYVKLFPNS |
| 6329 | | LDQTDMHGDSEYNIMFGPDICGPGTKKVHVIFNYKGKNVLINKDIRCKD |
| | | DEFTHLYTLIVRPDNTYEVKIDNSQVESGSLEDDWDFLPPKKIKDPDASK |
| | | PEDWDERAKIDDPTDSKPEDWDKPEHIPDPDAKKPEDWDEEMDGEWEP |
| | | PVIQNPEYKGEWKPRQIDNPDYKGTWIHPEIDNPEYSPDPSIYAYDNFGV |
| | | LGLDLWQVKSGTIFDNFLITNDEAYAEEFGNETWGVTKAAEKQMKDKQ |
| | | DEEQRLKRMMRTKMRMRRMRRTRRKMRRKMSPARPRTSCREACLQG |
| | | WTEA |
| SEQ | Type | EPAVYFKEQFLDGDGWTSRWIESKHKSDFGKFVLSSGKFYGDEEKDKGL |
| ID NO: | 19 | QTSQDARFYALSASFEPFSNKGQTLVVQFTVKHEQNIDCGGGYVKLFPNS |
| 6330 | | LDQTDMHGDSEYNIMFGPDICGPGTKKVHVIFNYKGKNVLINKDIRCKD |
| | | DEFTHLYTLIVRPDNTYEVKIDNSQVESGSLEDDWDFLPPKKIKDPDASK |
| | | PEDWDERAKIDDPTDSKPEDWDKPEHIPDPDAKKPEDWDEEMDGEWEP |
| | | PVIQNPEYKGEWKPRQIDNPDYKGTWIHPEIDNPEYSPDPSIYAYDNFGV |
| | | LGLDLWQVKSGTIFDNFLITNDEAYAEEFGNETWGVTKAAEKQMKDKQ |
| | | DEEQRLKEERQRTRRMMRTKMRMRRMRRTRRKMRRKMSPARPRTSCR |
| | | EACLQGWTEA |
| SEQ | Type | EPAVYFKEQFLDGDGWTSRWIESKHKSDFGKFVLSSGKFYGDEEKDKGL |
| ID NO: | 20 | QTSQDARFYALSASFEPFSNKGQTLVVQFTVKHEQNIDCGGGYVKLFPNS |
| 6331 | | LDQTDMHGDSEYNIMFGPDICGPGTKKVHVIFNYKGKNVLINKDIRCKD |
| | | DEFTHLYTLIVRPDNTYEVKIDNSQVESGSLEDDWDFLPPKKIKDPDASK |
| | | PEDWDERAKIDDPTDSKPEDWDKPEHIPDPDAKKPEDWDEEMDGEWEP |
| | | PVIQNPEYKGEWKPRQIDNPDYKGTWIHPEIDNPEYSPDPSIYAYDNFGV |
| | | LGLDLWQVKSGTIFDNFLITNDEAYAEEFGNETWGVTKAAEKQMKDKQ |
| | | DEEQRLKEEEEGRRQRTRRMMRTKMRMRRMRRTRRKMRRKMSPARPR |
| | | TSCREACLQGWTEA |
| SEQ | Type | EPAVYFKEQFLDGDGWTSRWIESKHKSDFGKFVLSSGKFYGDEEKDKGL |
| ID NO: | 21 | QTSQDARFYALSASFEPFSNKGQTLVVQFTVKHEQNIDCGGGYVKLFPNS |
| 6332 | | LDQTDMHGDSEYNIMFGPDICGPGTKKVHVIFNYKGKNVLINKDIRCKD |
| | | DEFTHLYTLIVRPDNTYEVKIDNSQVESGSLEDDWDFLPPKKIKDPDASK |
| | | PEDWDERAKIDDPTDSKPEDWDKPEHIPDPDAKKPEDWDEEMDGEWEP |
| | | PVIQNPEYKGEWKPRQIDNPDYKGTWIHPEIDNPEYSPDPSIYAYDNFGV |
| | | LGLDLWQVKSGTIFDNFLITNDEAYAEEFGNETWGVTKAAEKQMKDKQ |
| | | DEEQRLKEEEEAFKRTRRMMRTKMRMRRMRRTRRKMRRKMSPARPRT |
| | | SCREACLQGWTEA |
| SEQ | Type | EPAVYFKEQFLDGDGWTSRWIESKHKSDFGKFVLSSGKFYGDEEKDKGL |
| ID NO: | 22 | QTSQDARFYALSASFEPFSNKGQTLVVQFTVKHEQNIDCGGGYVKLFPNS |
| 6333 | | LDQTDMHGDSEYNIMFGPDICGPGTKKVHVIFNYKGKNVLINKDIRCKD |
| | | DEFTHLYTLIVRPDNTYEVKIDNSQVESGSLEDDWDFLPPKKIKDPDASK |
| | | PEDWDERAKIDDPTDSKPEDWDKPEHIPDPDAKKPEDWDEEMDGEWEP |
| | | PVIQNPEYKGEWKPRQIDNPDYKGTWIHPEIDNPEYSPDPSIYAYDNFGV |
| | | LGLDLWQVKSGTIFDNFLITNDEAYAEEFGNETWGVTKAAEKQMKDKQ |
| | | DEEQRLKEEEEDNAKRRRRQRTRRMMRTKMRMRRMRRTRRKMRRKM |
| | | SPARPRTSCREACLQGWTEA |
| SEQ | Type | EPAVYFKEQFLDGDGWTSRWIESKHKSDFGKFVLSSGKFYGDEEKDKGL |
| ID NO: | 23 | QTSQDARFYALSASFEPFSNKGQTLVVQFTVKHEQNIDCGGGYVKLFPNS |
| 6334 | | LDQTDMHGDSEYNIMFGPDICGPGTKKVHVIFNYKGKNVLINKDIRCKD |
| | | DEFTHLYTLIVRPDNTYEVKIDNSQVESGSLEDDWDFLPPKKIKDPDASK |
| | | PEDWDERAKIDDPTDSKPEDWDKPEHIPDPDAKKPEDWDEEMDGEWEP |
| | | PVIQNPEYKGEWKPRQIDNPDYKGTWIHPEIDNPEYSPDPSIYAYDNFGV |
| | | LGLDLWQVKSGTIFDNFLITNDEAYAEEFGNETWGVTKAAEKQMKDKQ |
| | | DEEQRLKEEEEDCVRRRRQRTRRMMRTKMRMRRMRRTRRKMRRKMSP |
| | | ARPRTSCREACLQGWTEA |
| SEQ | Type | EPAVYFKEQFLDGDGWTSRWIESKHKSDFGKFVLSSGKFYGDEEKDKGL |
| ID NO: | 24 | QTSQDARFYALSASFEPFSNKGQTLVVQFTVKHEQNIDCGGGYVKLFPNS |
| 6335 | | LDQTDMHGDSEYNIMFGPDICGPGTKKVHVIFNYKGKNVLINKDIRCKD |
| | | DEFTHLYTLIVRPDNTYEVKIDNSQVESGSLEDDWDFLPPKKIKDPDASK |
| | | PEDWDERAKIDDPTDSKPEDWDKPEHIPDPDAKKPEDWDEEMDGEWEP |
| | | PVIQNPEYKGEWKPRQIDNPDYKGTWIHPEIDNPEYSPDPSIYAYDNFGV |
| | | LGLDLWQVKSGTIFDNFLITNDEAYAEEFGNETWGVTKAAEKQMKDKQ |
| | | DEEQRLKEEEEDRRQRTRRMMRTKMRMRRMRRTRRKMRRKMSPARPR |
| | | TSCREACLQGWTEA |
| SEQ | Type | EPAVYFKEQFLDGDGWTSRWIESKHKSDFGKFVLSSGKFYGDEEKDKGL |
| ID NO: | 25 | QTSQDARFYALSASFEPFSNKGQTLVVQFTVKHEQNIDCGGGYVKLFPNS |
| 6336 | | LDQTDMHGDSEYNIMFGPDICGPGTKKVHVIFNYKGKNVLINKDIRCKD |
| | | DEFTHLYTLIVRPDNTYEVKIDNSQVESGSLEDDWDFLPPKKIKDPDASK |
| | | PEDWDERAKIDDPTDSKPEDWDKPEHIPDPDAKKPEDWDEEMDGEWEP |
| | | PVIQNPEYKGEWKPRQIDNPDYKGTWIHPEIDNPEYSPDPSIYAYDNFGV |
| | | LGLDLWQVKSGTIFDNFLITNDEAYAEEFGNETWGVTKAAEKQMKDKQ |
| | | DEEQRLKEEEEDKRQRTRRMMRTKMRMRRMRRTRRKMRRKMSPARPR |
| | | TSCREACLQGWTEA |
| SEQ | Type | EPAVYFKEQFLDGDGWTSRWIESKHKSDFGKFVLSSGKFYGDEEKDKGL |
| ID NO: | 26 | QTSQDARFYALSASFEPFSNKGQTLVVQFTVKHEQNIDCGGGYVKLFPNS |
| 6337 | | LDQTDMHGDSEYNIMFGPDICGPGTKKVHVIFNYKGKNVLINKDIRCKD |
| | | DEFTHLYTLIVRPDNTYEVKIDNSQVESGSLEDDWDFLPPKKIKDPDASK |
| | | PEDWDERAKIDDPTDSKPEDWDKPEHIPDPDAKKPEDWDEEMDGEWEP |
| | | PVIQNPEYKGEWKPRQIDNPDYKGTWIHPEIDNPEYSPDPSIYAYDNFGV |
| | | LGLDLWQVKSGTIFDNFLITNDEAYAEEFGNETWGVTKAAEKQMKDKQ |
| | | DEEQRLKEEEEDKNAKRRRRQRTRRMMRTKMRMRRMRRTRRKMRRK |
| | | MSPARPRTSCREACLQGWTEA |
| SEQ | Type | EPAVYFKEQFLDGDGWTSRWIESKHKSDFGKFVLSSGKFYGDEEKDKGL |
| ID NO: | 27 | QTSQDARFYALSASFEPFSNKGQTLVVQFTVKHEQNIDCGGGYVKLFPNS |
| 6338 | | LDQTDMHGDSEYNIMFGPDICGPGTKKVHVIFNYKGKNVLINKDIRCKD |
| | | DEFTHLYTLIVRPDNTYEVKIDNSQVESGSLEDDWDFLPPKKIKDPDASK |
| | | PEDWDERAKIDDPTDSKPEDWDKPEHIPDPDAKKPEDWDEEMDGEWEP |
| | | PVIQNPEYKGEWKPRQIDNPDYKGTWIHPEIDNPEYSPDPSIYAYDNFGV |
| | | LGLDLWQVKSGTIFDNFLITNDEAYAEEFGNETWGVTKAAEKQMKDKQ |
| | | DEEQRLKEEEEDKCFAKRRRRQRTRRMMRTKMRMRRMRRTRRKMRRK |
| | | MSPARPRTSCREACLQGWTEA |
| SEQ | Type | EPAVYFKEQFLDGDGWTSRWIESKHKSDFGKFVLSSGKFYGDEEKDKGL |
| ID NO: | 28 | QTSQDARFYALSASFEPFSNKGQTLVVQFTVKHEQNIDCGGGYVKLFPNS |
| 6339 | | LDQTDMHGDSEYNIMFGPDICGPGTKKVHVIFNYKGKNVLINKDIRCKD |
| | | DEFTHLYTLIVRPDNTYEVKIDNSQVESGSLEDDWDFLPPKKIKDPDASK |
| | | PEDWDERAKIDDPTDSKPEDWDKPEHIPDPDAKKPEDWDEEMDGEWEP |
| | | PVIQNPEYKGEWKPRQIDNPDYKGTWIHPEIDNPEYSPDPSIYAYDNFGV |
| | | LGLDLWQVKSGTIFDNFLITNDEAYAEEFGNETWGVTKAAEKQMKDKQ |
| | | DEEQRLKEEEEDKKRKRRMMRTKMRMRRMRRTRRKMRRKMSPARPRT |
| | | SCREACLQGWTEA |
| SEQ | Type | EPAVYFKEQFLDGDGWTSRWIESKHKSDFGKFVLSSGKFYGDEEKDKGL |
| ID NO: | 29 | QTSQDARFYALSASFEPFSNKGQTLVVQFTVKHEQNIDCGGGYVKLFPNS |
| 6340 | | LDQTDMHGDSEYNIMFGPDICGPGTKKVHVIFNYKGKNVLINKDIRCKD |
| | | DEFTHLYTLIVRPDNTYEVKIDNSQVESGSLEDDWDFLPPKKIKDPDASK |
| | | PEDWDERAKIDDPTDSKPEDWDKPEHIPDPDAKKPEDWDEEMDGEWEP |
| | | PVIQNPEYKGEWKPRQIDNPDYKGTWIHPEIDNPEYSPDPSIYAYDNFGV |
| | | LGLDLWQVKSGTIFDNFLITNDEAYAEEFGNETWGVTKAAEKQMKDKQ |
| | | DEEQRLKEEEEDKKRKEPPLCLRRMMRTKMRMRRMRRTRRKMRRKMS |
| | | PARPRTSCREACLQGWTEA |
| SEQ | Type | EPAVYFKEQFLDGDGWTSRWIESKHKSDFGKFVLSSGKFYGDEEKDKGL |
| ID NO: | 30 | QTSQDARFYALSASFEPFSNKGQTLVVQFTVKHEQNIDCGGGYVKLFPNS |
| 6341 | | LDQTDMHGDSEYNIMFGPDICGPGTKKVHVIFNYKGKNVLINKDIRCKD |
| | | DEFTHLYTLIVRPDNTYEVKIDNSQVESGSLEDDWDFLPPKKIKDPDASK |
| | | PEDWDERAKIDDPTDSKPEDWDKPEHIPDPDAKKPEDWDEEMDGEWEP |
| | | PVIQNPEYKGEWKPRQIDNPDYKGTWIHPEIDNPEYSPDPSIYAYDNFGV |
| | | LGLDLWQVKSGTIFDNFLITNDEAYAEEFGNETWGVTKAAEKQMKDKQ |
| | | DEEQRLKEEEEDKKRKEDHPCRRMMRTKMRMRRMRRTRRKMRRKMSP |
| | | ARPRTSCREACLQGWTEA |
| SEQ | Type | EPAVYFKEQFLDGDGWTSRWIESKHKSDFGKFVLSSGKFYGDEEKDKGL |
| ID NO: | 31 | QTSQDARFYALSASFEPFSNKGQTLVVQFTVKHEQNIDCGGGYVKLFPNS |
| 6342 | | LDQTDMHGDSEYNIMFGPDICGPGTKKVHVIFNYKGKNVLINKDIRCKD |
| | | DEFTHLYTLIVRPDNTYEVKIDNSQVESGSLEDDWDFLPPKKIKDPDASK |
| | | PEDWDERAKIDDPTDSKPEDWDKPEHIPDPDAKKPEDWDEEMDGEWEP |
| | | PVIQNPEYKGEWKPRQIDNPDYKGTWIHPEIDNPEYSPDPSIYAYDNFGV |
| | | LGLDLWQVKSGTIFDNFLITNDEAYAEEFGNETWGVTKAAEKQMKDKQ |
| | | DEEQRLKEEEEDKKRKEEEEAEGNCRRMMRTKMRMRRMRRTRRKMRR |
| | | KMSPARPRTSCREACLQGWTEA |
| SEQ | Type | EPAVYFKEQFLDGDGWTSRWIESKHKSDFGKFVLSSGKFYGDEEKDKGL |
| ID NO: | 32 | QTSQDARFYALSASFEPFSNKGQTLVVQFTVKHEQNIDCGGGYVKLFPNS |
| 6343 | | LDQTDMHGDSEYNIMFGPDICGPGTKKVHVIFNYKGKNVLINKDIRCKD |
| | | DEFTHLYTLIVRPDNTYEVKIDNSQVESGSLEDDWDFLPPKKIKDPDASK |
| | | PEDWDERAKIDDPTDSKPEDWDKPEHIPDPDAKKPEDWDEEMDGEWEP |
| | | PVIQNPEYKGEWKPRQIDNPDYKGTWIHPEIDNPEYSPDPSIYAYDNFGV |
| | | LGLDLWQVKSGTIFDNFLITNDEAYAEEFGNETWGVTKAAEKQMKDKQ |
| | | DEEQRLKEEEEDKKRKEEEEAEDCRRMMRTKMRMRRMRRTRRKMRRK |
| | | MSPARPRTSCREACLQGWTEA |
| SEQ | Type | EPAVYFKEQFLDGDGWTSRWIESKHKSDFGKFVLSSGKFYGDEEKDKGL |
| ID NO: | 33 | QTSQDARFYALSASFEPFSNKGQTLVVQFTVKHEQNIDCGGGYVKLFPNS |
| 6344 | | LDQTDMHGDSEYNIMFGPDICGPGTKKVHVIFNYKGKNVLINKDIRCKD |
| | | DEFTHLYTLIVRPDNTYEVKIDNSQVESGSLEDDWDFLPPKKIKDPDASK |
| | | PEDWDERAKIDDPTDSKPEDWDKPEHIPDPDAKKPEDWDEEMDGEWEP |
| | | PVIQNPEYKGEWKPRQIDNPDYKGTWIHPEIDNPEYSPDPSIYAYDNFGV |
| | | LGLDLWQVKSGTIFDNFLITNDEAYAEEFGNETWGVTKAAEKQMKDKQ |
| | | DEEQRLKEEEEDKKRKEEEEAEDKCRRMMRTKMRMRRMRRTRRKMRR |
| | | KMSPARPRTSCREACLQGWTEA |
| SEQ | Type | EPAVYFKEQFLDGDGWTSRWIESKHKSDFGKFVLSSGKFYGDEEKDKGL |
| ID NO: | 34 | QTSQDARFYALSASFEPFSNKGQTLVVQFTVKHEQNIDCGGGYVKLFPNS |
| 6345 | | LDQTDMHGDSEYNIMFGPDICGPGTKKVHVIFNYKGKNVLINKDIRCKD |
| | | DEFTHLYTLIVRPDNTYEVKIDNSQVESGSLEDDWDFLPPKKIKDPDASK |
| | | PEDWDERAKIDDPTDSKPEDWDKPEHIPDPDAKKPEDWDEEMDGEWEP |
| | | PVIQNPEYKGEWKPRQIDNPDYKGTWIHPEIDNPEYSPDPSIYAYDNFGV |
| | | LGLDLWQVKSGTIFDNFLITNDEAYAEEFGNETWGVTKAAEKQMKDKQ |
| | | DEEQRLKEEEEDKKRKEEEEAEDTCRRMMRTKMRMRRMRRTRRKMRR |
| | | KMSPARPRTSCREACLQGWTEA |
| SEQ | Type | EPAVYFKEQFLDGDGWTSRWIESKHKSDFGKFVLSSGKFYGDEEKDKGL |
| ID NO: | 35 | QTSQDARFYALSASFEPFSNKGQTLVVQFTVKHEQNIDCGGGYVKLFPNS |
| 6346 | | LDQTDMHGDSEYNIMFGPDICGPGTKKVHVIFNYKGKNVLINKDIRCKD |
| | | DEFTHLYTLIVRPDNTYEVKIDNSQVESGSLEDDWDFLPPKKIKDPDASK |
| | | PEDWDERAKIDDPTDSKPEDWDKPEHIPDPDAKKPEDWDEEMDGEWEP |
| | | PVIQNPEYKGEWKPRQIDNPDYKGTWIHPEIDNPEYSPDPSIYAYDNFGV |
| | | LGLDLWQVKSGTIFDNFLITNDEAYAEEFGNETWGVTKAAEKQMKDKQ |
| | | DEEQRLKEEEEDKKRKEEEEAEDICRRMMRTKMRMRRMRRTRRKMRR |
| | | KMSPARPRTSCREACLQGWTEA |
| SEQ | Type | EPAVYFKEQFLDGDGWTSRWIESKHKSDFGKFVLSSGKFYGDEEKDKGL |
| ID NO: | 36 | QTSQDARFYALSASFEPFSNKGQTLVVQFTVKHEQNIDCGGGYVKLFPNS |
| 6344 | | LDQTDMHGDSEYNIMFGPDICGPGTKKVHVIFNYKGKNVLINKDIRCKD |
| | | DEFTHLYTLIVRPDNTYEVKIDNSQVESGSLEDDWDFLPPKKIKDPDASK |
| | | PEDWDERAKIDDPTDSKPEDWDKPEHIPDPDAKKPEDWDEEMDGEWEP |
| | | PVIQNPEYKGEWKPRQIDNPDYKGTWIHPEIDNPEYSPDPSIYAYDNFGV |
| | | LGLDLWQVKSGTIFDNFLITNDEAYAEEFGNETWGVTKAAEKQMKDKQ |
| | | DEEQRLKEEEEDKKRKEEEEAEDKCRRMMRTKMRMRRMRRTRRKMRR |
| | | KMSPARPRTSCREACLQGWTEA |
| SEQ | mutCal | MLLSVPLLLGLLGLAVAHHHHHHHHGGGGSEPAVYFKEQFLDGDGWTS |
| ID NO: | R ins | RWIESKHKSDFGKFVLSSGKFYGDEEKDKGLQTSQDARFYALSASFEPFS |
| 1002 | | NKGQTLVVQFTVKHEQNIDCGGGYVKLFPNSLDQTDMHGDSEYNIMFG |
| | | PDICGPGTKKVHVIFNYKGKNVLINKDIRCKDDEFTHLYTLIVRPDNTYE |
| | | VKIDNSQVESGSLEDDWDFLPPKKIKDPDASKPEDWDERAKIDDPTDSKP |
| | | EDWDKPEHIPDPDAKKPEDWDEEMDGEWEPPVIQNPEYKGEWKPRQID |
| | | NPDYKGTWIHPEIDNPEYSPDPSIYAYDNFGVLGLDLWQVKSGTIFDNFLI |
| | | TNDEAYAEEFGNETWGVTKAAEKQMKDKQDEEQRLKEEEEDKKRKEE |
| | | EEAEDNCRRMMRTKMRMRRMRRTRRKMRRKMSPARPRTSCREACLQG |
| | | WTEA |
| SEQ | mutCal | MLLSVPLLLGLLGLAVAHHHHHHHHGGGGSEPAVYFKEQFLDGDGWTS |
| ID NO: | R del | RWIESKHKSDFGKFVLSSGKFYGDEEKDKGLQTSQDARFYALSASFEPFS |
| 1003 | | NKGQTLVVQFTVKHEQNIDCGGGYVKLFPNSLDQTDMHGDSEYNIMFG |
| | | PDICGPGTKKVHVIFNYKGKNVLINKDIRCKDDEFTHLYTLIVRPDNTYE |
| | | VKIDNSQVESGSLEDDWDFLPPKKIKDPDASKPEDWDERAKIDDPTDSKP |
| | | EDWDKPEHIPDPDAKKPEDWDEEMDGEWEPPVIQNPEYKGEWKPRQID |
| | | NPDYKGTWIHPEIDNPEYSPDPSIYAYDNFGVLGLDLWQVKSGTIFDNFLI |
| | | TNDEAYAEEFGNETWGVTKAAEKQMKDKQDEEQRTRRMMRTKMRMR |
| | | RMRRTRRKMRRKMSPARPRTSCREACLQGWTEA |

TABLE 3
The C-terminal amino acid sequences of calreticulin mutants

| SEQ ID | | C-terminal sequences of insertion/deletion frameshift mutations of |
|---|---|---|
| NO | Type | calreticulin |
| SEQ ID | Type 1 | TRRMMRTKMRMRRMRRTRRKMRRKMSPARPRTSCREACLQGWTE |
| NO: 6287 | | A |
| SEQ ID | Type 2 | NCRRMMRTKMRMRRMRRTRRKMRRKMSPARPRTSCREACLQGWT |
| NO: 6288 | | EA |
| SEQ ID | Type 3 | QRTRRMMRTKMRMRRMRRTRRKMRRKMSPARPRTSCREACLQGW |
| NO: 6289 | | TEA |
| SEQ ID | Type 4 | RRRQRTRRMMRTKMRMRRMRRTRRKMRRKMSPARPRTSCREACL |
| NO: 6290 | | QGWTEA |
| SEQ ID | Type 5 | GQRTRRMMRTKMRMRRMRRTRRKMRRKMSPARPRTSCREACLQG |
| NO: 6291 | | WTEA |
| SEQ ID | Type 6 | RRQRTRRMMRTKMRMRRMRRTRRKMRRKMSPARPRTSCREACLQ |
| NO: 6292 | | GWTEA |
| SEQ ID | Type 7 | RRMMRTKMRMRRMRRTRRKMRRKMSPARPRTSCREACLQGWTEA |
| NO: 6293 | | |
| SEQ ID | Type 8 | RRQRTRRMMRTKMRMRRMRRTRRKMRRKMSPARPRTSCREACLQ |
| NO: 6292 | | GWTEA |
| SEQ ID | Type 9 | RQRTRRMMRTKMRMRRMRRTRRKMRRKMSPARPRTSCREACLQG |
| NO: 6294 | | WTEA |
| SEQ ID | Type 10 | MCRRMMRTKMRMRRMRRTRRKMRRKMSPARPRTSCREACLQGWT |
| NO: 6295 | | EA |
| SEQ ID | Type 11 | DQRQRTRRMMRTKMRMRRMRRTRRKMRRKMSPARPRTSCREACL |
| NO: 6296 | | QGWTEA |
| SEQ ID | Type 12 | RRRRQRTRRMMRTKMRMRRMRRTRRKMRRKMSPARPRTSCREACL |
| NO: 152 | | QGWTEA |
| SEQ ID | Type 13 | QRRRQRTRRMMRTKMRMRRMRRTRRKMRRKMSPARPRTSCREAC |
| NO: 6297 | | LQGWTEA |
| SEQ ID | Type 14 | RRRQRTRRMMRTKMRMRRMRRTRRKMRRKMSPARPRTSCREACL |
| NO: 6290 | | QGWTEA |
| SEQ ID | Type 15 | RRRERTRRMMRTKMRMRRMRRTRRKMRRKMSPARPRTSCREACLQ |
| NO: 6298 | | GWTEA |
| SEQ ID | Type 16 | QRRQRTRRMMRTKMRMRRMRRTRRKMRRKMSPARPRTSCREACL |
| NO: 6299 | | QGWTEA |
| SEQ ID | Type 17 | RRQWTRRMMRTKMRMRRMRRTRRKMRRKMSPARPRTSCREACLQ |
| NO: 6300 | | GWTEA |
| SEQ ID | Type 18 | RMMRTKMRMRRMRRTRRKMRRKMSPARPRTSCREACLQGWTEA |
| NO: 6301 | | |
| SEQ ID | Type 19 | RQRTRRMMRTKMRMRRMRRTRRKMRRKMSPARPRTSCREACLQG |
| NO: 6294 | | WTEA |
| SEQ ID | Type 20 | GRRQRTRRMMRTKMRMRRMRRTRRKMRRKMSPARPRTSCREACL |
| NO: 6302 | | QGWTEA |
| SEQ ID | Type 21 | AFKRTRRMMRTKMRMRRMRRTRRKMRRKMSPARPRTSCREACLQ |
| NO: 6303 | | GWTEA |
| SEQ ID | Type 22 | NAKRRRRQRTRRMMRTKMRMRRMRRTRRKMRRKMSPARPRTSCR |
| NO: 6304 | | EACLQGWTEA |
| SEQ ID | Type 23 | CVRRRRQRTRRMMRTKMRMRRMRRTRRKMRRKMSPARPRTSCRE |
| NO: 6305 | | ACLQGWTEA |
| SEQ ID | Type 24 | RRQRTRRMMRTKMRMRRMRRTRRKMRRKMSPARPRTSCREACLQ |
| NO: 6292 | | GWTEA |
| SEQ ID | Type 25 | RQRTRRMMRTKMRMRRMRRTRRKMRRKMSPARPRTSCREACLQG |
| NO: 6294 | | WTEA |
| SEQ ID | Type 26 | NAKRRRRQRTRRMMRTKMRMRRMRRTRRKMRRKMSPARPRTSCR |
| NO: 6304 | | EACLQGWTEA |
| SEQ ID | Type 27 | CFAKRRRRQRTRRMMRTKMRMRRMRRTRRKMRRKMSPARPRTSC |
| NO: 6306 | | REACLQGWTEA |
| SEQ ID | Type 28 | RRMMRTKMRMRRMRRTRRKMRRKMSPARPRTSCREACLQGWTEA |
| NO: 6293 | | |
| SEQ ID | Type 29 | PPLCLRRMMRTKMRMRRMRRTRRKMRRKMSPARPRTSCREACLQG |
| NO: 6307 | | WTEA |
| SEQ ID | Type 30 | DHPCRRMMRTKMRMRRMRRTRRKMRRKMSPARPRTSCREACLQG |
| NO: 6308 | | WTEA |
| SEQ ID | Type 31 | GNCRRMMRTKMRMRRMRRTRRKMRRKMSPARPRTSCREACLQGW |
| NO: 6309 | | TEA |
| SEQ ID | Type 32 | CRRMMRTKMRMRRMRRTRRKMRRKMSPARPRTSCREACLQGWTE |
| NO: 6310 | | A |
| SEQ ID | Type 33 | CRRMMRTKMRMRRMRRTRRKMRRKMSPARPRTSCREACLQGWTE |
| NO: 6310 | | A |
| SEQ ID | Type 34 | TCRRMMRTKMRMRRMRRTRRKMRRKMSPARPRTSCREACLQGWT |
| NO: 6311 | | EA |
| SEQ ID | Type 35 | ICRRMMRTKMRMRRMRRTRRKMRRKMSPARPRTSCREACLQGWTE |
| NO: 6312 | | A |
| SEQ ID | Type 36 | CRRMMRTKMRMRRMRRTRRKMRRKMSPARPRTSCREACLQGWTE |
| NO: 6310 | | A |

In some embodiments, the calreticulin-targeting antigen binding domain comprises any CDR amino acid sequence, framework region (FWR) amino acid sequence, or variable region amino acid sequence disclosed in Tables 4-7, Table 24, and Table 25.

In some embodiments, the calreticulin-targeting antigen binding domain comprises a VH comprising one, two, three CDRs from murine 16B11.1 antibody, e.g., as described in Table 4. For example, in some embodiments, the calreticulin-targeting antigen binding domain comprises a VH comprising a heavy chain complementarity determining region 1 (VHCDR1) amino acid sequence of SEQ ID NO: 6358 (or a sequence with no more than 1, 2, 3, or 4 mutations, e.g., substitutions, additions, or deletions), a VHCDR2 amino acid sequence of SEQ ID NO: 6360 (or a sequence with no more than 1, 2, 3, or 4 mutations, e.g., substitutions, additions, or deletions), and/or a VHCDR3 amino acid sequence of SEQ ID NO: 227 (or a sequence with no more than 1, 2, 3, or 4 mutations, e.g., substitutions, additions, or deletions). In some embodiments, the calreticulin-targeting antigen binding domain comprises a VH comprising a heavy chain complementarity determining region 1 (VHCDR1) amino acid sequence of SEQ ID NO: 6358 (or a sequence with no more than 1, 2, 3, or 4 mutations, e.g., substitutions, additions, or deletions), a VHCDR2 amino acid sequence of SEQ ID NO: 6360 (or a sequence with no more than 1, 2, 3, or 4 mutations, e.g., substitutions, additions, or deletions), and/or a VHCDR3 amino acid sequence of SEQ ID NO: 227 (or a sequence with no more than 1, 2, 3, or 4 mutations, e.g., substitutions, additions, or deletions). In some embodiments, the calreticulin-targeting antigen binding domain comprises a VH comprising a heavy chain complementarity determining region 1 (VHCDR1) amino acid sequence of SEQ ID NO: 6358 (or a sequence with no more than 1, 2, 3, or 4 mutations, e.g., substitutions, additions, or deletions), a VHCDR2 amino acid sequence of SEQ ID NO: 6360 (or a sequence with no more than 1, 2, 3, or 4 mutations, e.g., substitutions, additions, or deletions), and/or a VHCDR3 amino acid sequence of SEQ ID NO: 227 (or a sequence with no more than 1, 2, 3, or 4 mutations, e.g., substitutions, additions, or deletions). In some embodiments, the calreticulin-targeting antigen binding domain comprises a VH comprising a heavy chain complementarity determining region 1 (VHCDR1) amino acid sequence of SEQ ID NO: 6358 (or a sequence with no more than 1, 2, 3, or 4 mutations, e.g., substitutions, additions, or deletions), a VHCDR2 amino acid sequence of SEQ ID NO: 6360 (or a sequence with no more than 1, 2, 3, or 4 mutations, e.g., substitutions, additions, or deletions), and/or a VHCDR3 amino acid sequence of SEQ ID NO: 227 (or a sequence with no more than 1, 2, 3, or 4 mutations, e.g., substitutions, additions, or deletions).

Alternatively, or in combination with the calreticulin-targeting antigen binding domain comprising the VH comprising one, two, three CDRs from murine 16B11.1 antibody, the calreticulin-targeting antigen binding domain comprises a VL comprising one, two or three CDRs derived from murine 16B11.1 antibody, e.g., as described in Table 4. For example, in some embodiments, the calreticulin-targeting antigen binding domain comprises a VL comprising a light chain complementarity determining region 1 (VLCDR1) amino acid sequence of SEQ ID NO: 251 (or a sequence with no more than 1, 2, 3, or 4 mutations, e.g., substitutions, additions, or deletions), a VLCDR2 amino acid sequence of SEQ ID NO: 246 (or a sequence with no more than 1, 2, 3, or 4 mutations, e.g., substitutions, additions, or deletions), and/or a VLCDR3 amino acid sequence of SEQ ID NO: 248 (or a sequence with no more than 1, 2, 3, or 4 mutations, e.g., substitutions, additions, or deletions). In some embodiments, the calreticulin-targeting antigen binding domain comprises a VL comprising a VLCDR1 amino acid sequence of SEQ ID NO: 251, a VLCDR2 amino acid sequence of SEQ ID NO: 253, and a VLCDR3 amino acid sequence of SEQ ID NO: 255. In some embodiments, the calreticulin-targeting antigen binding domain comprises a VL comprising a VLCDR1 amino acid sequence of SEQ ID NO: 258, a VLCDR2 amino acid sequence of SEQ ID NO: 260, and a VLCDR3 amino acid sequence of SEQ ID NO: 262. In some embodiments, the calreticulin-targeting antigen binding domain comprises a VL comprising a VLCDR1 amino acid sequence of SEQ ID NO: 265, a VLCDR2 amino acid sequence of SEQ ID NO: 267, and a VLCDR3 amino acid sequence of SEQ ID NO: 269. In some embodiments, the calreticulin-targeting antigen binding domain comprises a VL comprising a VLCDR1 amino acid sequence of SEQ ID NO: 272, a VLCDR2 amino acid sequence of SEQ ID NO: 274, and a VLCDR3 amino acid sequence of SEQ ID NO: 276. In some embodiments, the calreticulin-targeting antigen binding domain comprises a VL comprising a VLCDR1 amino acid sequence of SEQ ID NO: 279, a VLCDR2 amino acid sequence of SEQ ID NO: 281, and a VLCDR3 amino acid sequence of SEQ ID NO: 283.

In some embodiments, the calreticulin-targeting antigen binding domain comprises a VH comprising a heavy chain complementarity determining region 1 (VHCDR1) amino acid sequence of SEQ ID NO: 6253 (or a sequence with no more than 1, 2, 3, or 4 mutations, e.g., substitutions, additions, or deletions), a VHCDR2 amino acid sequence of SEQ ID NO: 6254 (or a sequence with no more than 1, 2, 3, or 4 mutations, e.g., substitutions, additions, or deletions), and/or a VHCDR3 amino acid sequence of SEQ ID NO: 6255 (or a sequence with no more than 1, 2, 3, or 4 mutations, e.g., substitutions, additions, or deletions). In some embodiments, the calreticulin-targeting antigen binding domain comprises a VH comprising a VHCDR1 amino acid sequence of SEQ ID NO: 6253, a VHCDR2 amino acid sequence of SEQ ID NO: 6254, and/or a VHCDR3 amino acid sequence of SEQ ID NO: 6255.

In some embodiments, the calreticulin-targeting antigen binding domain comprises a VL comprising a light chain complementarity determining region 1 (VLCDR1) amino acid sequence of SEQ ID NO: 6259 (or a sequence with no more than 1, 2, 3, or 4 mutations, e.g., substitutions, additions, or deletions), a VLCDR2 amino acid sequence of SEQ ID NO: 6260 (or a sequence with no more than 1, 2, 3, or 4 mutations, e.g., substitutions, additions, or deletions), and/or a VLCDR3 amino acid sequence of SEQ ID NO: 6261 (or a sequence with no more than 1, 2, 3, or 4 mutations, e.g., substitutions, additions, or deletions). In some embodiments, the calreticulin-targeting antigen binding domain comprises a VL comprising a VLCDR1 amino acid sequence of SEQ ID NO: 6259, a VLCDR2 amino acid sequence of SEQ ID NO: 6260, and a VLCDR3 amino acid sequence of SEQ ID NO: 6261.

In some embodiments, the calreticulin-targeting antigen binding domain comprises a VH comprising one, two, three, or four framework regions from humanized 16B11.1 antibody, e.g., as described in Table 4. For example, in some embodiments, the calreticulin-targeting antigen binding domain comprises a VH comprising a heavy chain framework region 1 (VHFWR1) amino acid sequence of SEQ ID NO: 6357, a VHFWR2 amino acid sequence of SEQ ID NO: 6359, a VHFWR3 amino acid sequence of SEQ ID NO: 6361, and/or a VHFWR4 amino acid sequence of SEQ ID NO: 6273. In some embodiments, the calreticulin-targeting antigen binding domain comprises a VH comprising a heavy chain framework region 1 (VHFWR1) amino acid sequence of SEQ ID NO: 6362, a VHFWR2 amino acid sequence of SEQ ID NO: 6363, a VHFWR3 amino acid sequence of SEQ ID NO: 226, and/or a VHFWR4 amino acid sequence of SEQ ID NO: 228. In some embodiments, the calreticulin-targeting antigen binding domain comprises a VH comprising a heavy chain framework region 1 (VHFWR1) amino acid sequence of SEQ ID NO: 229, a VHFWR2 amino acid sequence of SEQ ID NO: 6369, a VHFWR3 amino acid sequence of SEQ ID NO: 6371, and/or a VHFWR4 amino acid sequence of SEQ ID NO: 228. In some embodiments, the calreticulin-targeting antigen binding domain comprises a VH comprising a heavy chain framework region 1 (VHFWR1) amino acid sequence of SEQ ID NO: 6373, a VHFWR2 amino acid sequence of SEQ ID NO: 6369, a VHFWR3 amino acid sequence of SEQ ID NO: 6371, and/or a VHFWR4 amino acid sequence of SEQ ID NO: 228.

In some embodiments, the calreticulin-targeting antigen binding domain comprises a VL comprising a light chain framework region 1 (VLFWR1) amino acid sequence of SEQ ID NO: 6374, a VLFWR2 amino acid sequence of SEQ ID NO: 6375, a VLFWR3 amino acid sequence of SEQ ID NO: 247, and/or a VLFWR4 amino acid sequence of SEQ ID NO: 249. In some embodiments, the calreticulin-targeting antigen binding domain comprises a VL comprising a light chain framework region 1 (VLFWR1) amino acid sequence of SEQ ID NO: 250, a VLFWR2 amino acid sequence of SEQ ID NO: 252, a VLFWR3 amino acid sequence of SEQ ID NO: 254, and/or a VLFWR4 amino acid sequence of SEQ ID NO: 256. In some embodiments, the calreticulin-targeting antigen binding domain comprises a VL comprising a light chain framework region 1 (VLFWR1) amino acid sequence of SEQ ID NO: 257, a VLFWR2 amino acid sequence of SEQ ID NO: 259, a VLFWR3 amino acid sequence of SEQ ID NO: 261, and/or a VLFWR4 amino acid sequence of SEQ ID NO: 263. In some embodiments, the calreticulin-targeting antigen binding domain comprises a VL comprising a light chain framework region 1 (VLFWR1) amino acid sequence of SEQ ID NO: 264, a VLFWR2 amino acid sequence of SEQ ID NO: 266, a VLFWR3 amino acid sequence of SEQ ID NO: 268, and/or a VLFWR4 amino acid sequence of SEQ ID NO: 270. In some embodiments, the calreticulin-targeting antigen binding domain comprises a VL comprising a light chain framework region 1 (VLFWR1) amino acid sequence of SEQ ID NO: 271, a VLFWR2 amino acid sequence of SEQ ID NO: 273, a VLFWR3 amino acid sequence of SEQ ID NO: 275, and/or a VLFWR4 amino acid sequence of SEQ ID NO: 277. In some embodiments, the calreticulin-targeting antigen binding domain comprises a VL comprising a light chain framework region 1 (VLFWR1) amino acid sequence of SEQ ID NO: 278, a VLFWR2 amino acid sequence of SEQ ID NO: 280, a VLFWR3 amino acid sequence of SEQ ID NO: 282, and/or a VLFWR4 amino acid sequence of SEQ ID NO: 284.

In some embodiments, the calreticulin-targeting antigen binding domain comprises a VH comprising a heavy chain framework region 1 (VHFWR1) amino acid sequence of SEQ ID NO: 6224, a VHFWR2 amino acid sequence of SEQ ID NO: 6226, a VHFWR3 amino acid sequence of SEQ ID NO: 6228, and/or a VHFWR4 amino acid sequence of SEQ ID NO: 6230. In some embodiments, the calreticulin-targeting antigen binding domain comprises a VL comprising a light chain framework region 1 (VLFWR1) amino acid sequence of SEQ ID NO: 6238, a VLFWR2 amino acid sequence of SEQ ID NO: 6240, a VLFWR3 amino acid sequence of SEQ ID NO: 6242, and/or a VLFWR4 amino acid sequence of SEQ ID NO: 6244. In some embodiments, the calreticulin-targeting antigen binding domain comprises a VH comprising a VHFWR1 amino acid sequence of SEQ ID NO: 6263 (or a sequence with no more than 1, 2, 3, 4, 5, or 6 mutations, e.g., substitutions, additions, or deletions), a VHFWR2 amino acid sequence of SEQ ID NO: 6264 (or a sequence with no more than 1, 2, 3, 4, 5, or 6 mutations, e.g., substitutions, additions, or deletions), a VHFWR3 amino acid sequence of SEQ ID NO: 6265 (or a sequence with no more than 1, 2, 3, 4, 5, 6, 7, 8, 9, 10, or 11 mutations, e.g., substitutions, additions, or deletions), and/or a VHFWR4 amino acid sequence of SEQ ID NO: 228. In some embodiments, the calreticulin-targeting antigen binding domain comprises a VL comprising a VLFWR1 amino acid sequence of SEQ ID NO: 6277 (or a sequence with no more than 1, 2, or 3 mutations, e.g., substitutions, additions, or deletions), a VLFWR2 amino acid sequence of SEQ ID NO: 6278 (or a sequence with no more than 1 mutation, e.g., substitution, addition, or deletion), a VLFWR3 amino acid sequence of SEQ ID NO: 6279 (or a sequence with no more than 1 mutation, e.g., substitution, addition, or deletion), and/or a VLFWR4 amino acid sequence of SEQ ID NO: 6280. In some embodiments, the calreticulin-targeting antigen binding domain comprises a VH comprising a VHFWR1 amino acid sequence of SEQ ID NO: 6263, a VHFWR2 amino acid sequence of SEQ ID NO: 6264, a VHFWR3 amino acid sequence of SEQ ID NO: 6265, and/or a VHFWR4 amino acid sequence of SEQ ID NO: 228. In some embodiments, the calreticulin-targeting antigen binding domain comprises a VL comprising a VLFWR1 amino acid sequence of SEQ ID NO: 6277, a VLFWR2 amino acid sequence of SEQ ID NO: 6278, a VLFWR3 amino acid sequence of SEQ ID NO: 6279, and/or a VLFWR4 amino acid sequence of SEQ ID NO: 6280.

In some embodiments, the calreticulin-targeting antigen binding domain comprises a VH comprising the amino acid sequence of SEQ ID NO: 6347 (or an amino acid sequence having at least about 77%, 80%, 85%, 90%, 95%, or 99% sequence identity to SEQ ID NO: 6347). In some embodiments, the calreticulin-targeting antigen binding domain comprises a VL comprising the amino acid sequence of SEQ ID NO: 6348 (or an amino acid sequence having at least about 77%, 80%, 85%, 90%, 95%, or 99% sequence identity to SEQ ID NO: 6348). In some embodiments, the calreticulin-targeting antigen binding domain comprises a VH comprising the amino acid sequence of SEQ ID NO: 6349 (or an amino acid sequence having at least about 77%, 80%, 85%, 90%, 95%, or 99% sequence identity to SEQ ID NO: 6349). In some embodiments, the calreticulin-targeting antigen binding domain comprises a VH comprising the amino acid sequence of SEQ ID NO: 6350 (or an amino acid sequence having at least about 77%, 80%, 85%, 90%, 95%, or 99% sequence identity to SEQ ID NO: 6350). In some embodiments, the calreticulin-targeting antigen binding domain comprises a VH comprising the amino acid sequence of SEQ ID NO: 6351 (or an amino acid sequence having at least about 77%, 80%, 85%, 90%, 95%, or 99% sequence identity to SEQ ID NO: 6351). In some embodiments, the calreticulin-targeting antigen binding domain comprises a VL comprising the amino acid sequence of SEQ ID NO: 6352 (or an amino acid sequence having at least about 93%, 95%, or 99% sequence identity to SEQ ID NO: 6352). In some embodiments, the calreticulin-targeting antigen binding domain comprises a VL comprising the amino acid sequence of SEQ ID NO: 6353 (or an amino acid sequence having at least about 93%, 95%, or 99% sequence identity to SEQ ID NO: 6353). In some embodiments, the calreticulin-targeting antigen binding domain comprises a VL comprising the amino acid sequence of SEQ ID NO: 6354 (or an amino acid sequence having at least about 93%, 95%, or 99% sequence identity to SEQ ID NO: 6354). In some embodiments, the calreticulin-targeting antigen binding domain comprises a VL comprising the amino acid sequence of SEQ ID NO: 6355 (or an amino acid sequence having at least about 93%, 95%, or 99% sequence identity to SEQ ID NO: 6355). In some embodiments, the calreticulin-targeting antigen binding domain comprises a VL comprising the amino acid sequence of SEQ ID NO: 6356 (or an amino acid sequence having at least about 93%, 95%, or 99% sequence identity to SEQ ID NO: 6356).

In some embodiments, the calreticulin-targeting antigen binding domain comprises a VH comprising the amino acid sequence of SEQ ID NO: 6247 (or an amino acid sequence having at least about 77%, 80%, 85%, 90%, 95%, or 99% sequence identity to SEQ ID NO: 6247). In some embodiments, the calreticulin-targeting antigen binding domain comprises a VL comprising the amino acid sequence of SEQ ID NO: 6249 (or an amino acid sequence having at least about 93%, 95%, or 99% sequence identity to SEQ ID NO: 6249). In some embodiments, the calreticulin-targeting antigen binding domain comprises a VH comprising the amino acid sequence of SEQ ID NO: 6247. In some embodiments, the calreticulin-targeting antigen binding domain comprises a VL comprising the amino acid sequence of SEQ ID NO: 6249. In some embodiments, the calreticulin-targeting antigen binding domain comprises a VH comprising the amino acid sequence of SEQ ID NO: 6247, and a VL comprising the amino acid sequence of SEQ ID NO: 6249.

In some embodiments, the calreticulin-targeting antigen binding domain comprises a VH comprising one, two, or all three CDR sequence as listed in a single row of Table 4, or an amino acid sequence having at least 75%, 80%, 85%, 90%, 95%, 96%, 97%, 98%, or 99% sequence identity thereto (e.g., to one, two, or all three of the CDR sequences). In some embodiments, the calreticulin-targeting antigen binding domain comprises a VL comprising one, two, or all three CDR sequence as listed in a single row of Table 5, or an amino acid sequence having at least 75%, 80%, 85%, 90%, 95%, 96%, 97%, 98%, or 99% sequence identity thereto (e.g., to one, two, or all three of the CDR sequences). In some embodiments, the calreticulin-targeting antigen binding domain comprises: (i) a VH comprising one, two, or all three CDR sequence as listed in a single row of Table 4, or an amino acid sequence having at least 75%, 80%, 85%, 90%, 95%, 96%, 97%, 98%, or 99% sequence identity thereto (e.g., to one, two, or all three of the CDR sequences); and (ii) a VL comprising one, two, or all three CDR sequence as listed in a single row of Table 5, or an amino acid sequence having at least 75%, 80%, 85%, 90%, 95%, 96%, 97%, 98%, or 99% sequence identity thereto (e.g., to one, two, or all three of the CDR sequences).

TABLE 4
Exemplary heavy chain CDRs and FWRs of calreticulin-targeting antigen binding domains

| Ab ID | VHFWR1 | VHCDR1 | VHFWR2 | VHCDR2 | VHFWR3 | VHCDR3 | VHFWR4 |
|---|---|---|---|---|---|---|---|
| AbH-1H | QVQLVQ | YSFTG | WVRQAP | YISCYN | RVTMTVDT | SSMDY | WGQG |
| | SGAEVK | YYIH | GQELGW | GASSY | SISTAYTEL | (SEQ | TLVTV |
| | KPGASVK | (SEQ | MG (SEQ | NQKFK | SSLRSEDT | ID NO: | SS (SEQ |
| | VSCKASG | ID NO: | ID NO: | G (SEQ | ATYYCA | 6255) | ID NO: |
| | (SEQ ID | 6253) | 6264) | ID NO: | (SEQ ID NO: | | 228) |
| | NO: 6263) | | | 6254) | 6265) | | |
| AbH-2H | QVTLKES | YSITSD | WIRQPP | YISYSG | RLSITKDTS | DPPYY | WGQG |
| | GPVLVKP | YAWN | GKALEW | STSYNP | KSQVVLTM | YGS | TTVTV |
| | TETLTLT | (SEQ | LA (SEQ | SLKS | TNMDPVDT | (SEQ | SS (SEQ |
| | CTVSG | ID NO: | ID NO: | (SEQ ID | ATYYCAR | ID NO: | ID NO: |
| | (SEQ ID | 6256) | 6267) | NO: | (SEQ ID NO: | 6258) | 6269) |
| | NO: 6266) | | | 6257) | 6268) | | |
| AbM-1H | EVQLEQS | YSFTG | WVKQS | YISCYN | KATFTVDT | SSMDY | WGQG |
| | GPELVKT | YYIH | HGKSLE | GASSY | SSSTAYMQ | (SEQ | TSVTV |
| | GASVKIS | (SEQ | WIG | NQKFK | FNSLTSGD | ID NO: | SS (SEQ |
| | CKASG | ID NO: | (SEQ ID | G (SEQ | SAVYYCA | 6255) | ID NO: |
| | (SEQ ID | 6253) | NO: 6271) | ID NO: | (SEQ ID NO: | | 6273) |
| | NO: 6270) | | | 6254) | 6272) | | |
| AbM-2H | DVQLQES | YSITSD | WIRQFP | YISYSG | RISITRDTS | DPPYY | WGQG |
| | GPGLVK | YAWN | GNKLEW | STSYNP | KNQFFLQL | YGSNG | TSVTV |
| | NSQSLSL | (SEQ | MG (SEQ | SLKS | NSVTPEDT | T (SEQ | SS (SEQ |
| | TCTVTG | ID NO: | ID NO: | (SEQ ID | ATYYCAR | ID NO: | ID NO: |
| | (SEQ ID | 6256) | 6275) | NO: | (SEQ ID NO: | 6262) | 6273) |
| | NO: 6274) | | | 6257) | 6276) | | |
| Murine | EVKLVES | FSRYD | WVRQTP | TISSGG | RFTISRDNA | HSAYY | WGQG |
| anti- | GGGLVK | MS | EKRLEW | SYTYYP | RNTLYLQM | VNYEN | TSVTV |
| calreticulin | PGGSLKL | (SEQ | VA (SEQ | DSVKG | SSLRSEDT | AMDY | SS (SEQ |
| antibody | SCAASGF | ID NO: | ID NO: | (SEQ ID | ALYYCAR | (SEQ | ID NO: |
| 16B11.1 | A | 6358) | 6359) | NO: | (SEQ ID NO: | ID NO: | 6273) |
| heavy chain | (SEQ ID | | | 6360) | 6361) | 227) | |
| variable | NO: 6357) | | | | | | |
| region | | | | | | | |
| Humanized | QVQLVES | FSRYD | WIRQAP | TISSGG | RFTISRDNA | HSAYY | WGQG |
| anti- | GGGLVK | MS | GKGLEW | SYTYYP | KNSLYLQM | VNYEN | TLVTV |
| calreticulin | PGGSLRL | (SEQ | VA (SEQ | DSVKG | NSLRAEDT | AMDY | SS |
| heavy chain | SCAASGF | ID NO: | ID NO: | (SEQ ID | AVYYCAR | (SEQ | (SEQ ID |
| variable | A (SEQ ID | 6358) | 6363) | NO: | (SEQ ID NO: | ID NO: | NO: |
| region | NO: 6362) | | | 6360) | 226) | 227) | 228) |
| variant 1 | | | | | | | |
| Humanized | QVQLVES | FSRYD | WVRQAP | TISSGG | RFTISRDNS | HSAYY | WGQG |
| anti- | GGGVVQ | MS | GKGLEW | SYTYYP | KNTLYLQ | VNYEN | TLVTV |
| calreticulin | PGRSLRL | (SEQ | VA (SEQ | DSVKG | MNSLRAED | AMDY | SS (SEQ |
| heavy chain | SCAASGF | ID NO: | ID NO: | (SEQ ID | TAVYYCAR | (SEQ | ID NO: |
| variable | A | 6358) | 6369) | NO: | (SEQ ID NO: | ID NO: | 228) |
| region | (SEQ ID | | | 6360) | 6371) | 227) | |
| variant 2 | NO: 229) | | | | | | |
| Humanized | EVQLVES | FSRYD | WVRQAP | TISSGG | RFTISRDNS | HSAYY | WGQG |
| anti- | GGGLVQ | MS | GKGLEW | SYTYYP | KNTLYLQ | VNYEN | TLVTV |
| calreticulin | PGGSLRL | (SEQ | VA (SEQ | DSVKG | MNSLRAED | AMDY | SS (SEQ |
| heavy chain | SCAASGF | ID NO: | ID NO: | (SEQ ID | TAVYYCAR | (SEQ | ID NO: |
| variable | A (SEQ ID | 6358) | 6369) | NO: | (SEQ ID NO: | ID NO: | 228) |
| region | NO: 6373) | | | 6360) | 6371) | 227) | |
| variant 3 | | | | | | | |
| 6C10 | EVQLVEK | FSEYW | WLRQAP | VIKYK | RFTISRDDS | GRDV | WGQG |
| Parental | GGGLVQ | MN | GKGLEW | YSNYA | KSSVYLQM | QDY | TMVTV |
| VH BK051 | PGKSLKL | (SEQ | VG (SEQ | TEFAES | TNLRAEDT | (SEQ | SS (SEQ |
| | SCTASGF | ID NO: | ID NO: | VKG | AIYYCAR | ID NO: | ID NO: |
| | T (SEQ ID | 7396) | 7398) | (SEQ ID | (SEQ ID NO: | 7403) | 6006) |
| | NO: 7394) | | | NO: | 7402) | | |
| | | | | 7400) | | | |
| 6C10 | EVQLVES | FSEYW | WLRQAP | VIKYK | RFTISRDDS | GRDV | WGQG |
| humanized | GGGLVQ | MN | GKGLEW | YSNYA | KSIVYLQM | QDY | TMVTV |
| heavy chain | PGPSLRL | (SEQ | VG (SEQ | TEFAES | NSLKTEDT | (SEQ | SS (SEQ |
| variable | SCTASGF | ID NO: | ID NO: | VKG | AVYYCAR | ID NO: | ID NO: |
| region | T (SEQ ID | 7396) | 7398) | (SEQ ID | (SEQ ID NO: | 7403) | 6006) |
| variant 1 | NO: 7423) | | | NO: | 7424) | | |
| (BK197) | | | | 7400) | | | |
| 6C10_hum1_ | EVQLVES | FSEYW | WLRQAP | VIKYK | RFTISRDDS | GRDV | WGQG |
| scFv VH | GGGLVQ | MN | GKGLEW | YSNYA | KSIVYLQM | QDY | TMVTV |
| (BKM0161) | PGRSLRL | (SEQ | VG (SEQ | TEFAES | NSLKTEDT | (SEQ | SS (SEQ |
| | SCTASGF | ID NO: | ID NO: | VKG | AVYYCAR | ID NO: | ID NO: |
| | T (SEQ ID | 7396) | 7398) | (SEQ ID | (SEQ ID NO: | 7403) | 6006) |
| | NO: 7436) | | | NO: | 7424) | | |
| | | | | 7400) | | | |
| 6C10_hum5_ | EVQLVES | SEYW | WLRQAP | VIKYK | RFTISRDDS | GRDV | WGQG |
| scFv VH | GGGLVQ | MN | GKGLEW | YSNYA | KSSVYLQM | QDY | TMVTV |
| (BKM0165) | PGGSLRL | (SEQ | VG (SEQ | TEFAES | NSLKTEDT | (SEQ | SS (SEQ |
| | SCAASGF | ID NO: | ID NO: | VKG | AVYYCAR | ID NO: | ID NO: |
| | TF (SEQ | 7444) | 7398) | (SEQ ID | (SEQ ID NO: | 7403) | 6006) |
| | ID NO: | | | NO: | 7445) | | |
| | 7443) | | | 7400) | | | |

TABLE 5
Exemplary light chain CDRs and FWRs of calreticulin-targeting antigen binding domains

| Ab ID | FWR1 | CDR1 | FWR2 | CDR2 | FWR3 | CDR3 | FWR4 |
|---|---|---|---|---|---|---|---|
| AbH-1L / | DVVMTQ | KSSQSLL | WLQQRP | LVSKL | GVPDRFSG | WQGTH | FGGG |
| AbH-2L | SPLSLPV | DSDGKT | GQSPRR | DS | SGSGTDFT | FPYT | TKVEI |
| | TLGQPAS | YLN | LIY (SEQ | (SEQ | LKISRVEA | (SEQ ID | K (SEQ |
| | ISC (SEQ | (SEQ ID | ID NO: | ID NO: | EDVGVYH | NO: | ID NO: |
| | ID NO: | NO: 6259) | 6278) | 6260) | C (SEQ ID | 6261) | 6280) |
| | 6277) | | | | NO: 6279) | | |
| AbM-1L / | DVVMTQ | KSSQSLL | WLLQRP | LVSKL | GVPDRFTG | WQGTH | FGGG |
| AbM-2L | TPLTLSV | DSDGKT | GQSPKR | DS | SGSGTDFT | FPYT | TKLEI |
| | TIGQPASI | YLN | LIY (SEQ | (SEQ | LKISRVEA | (SEQ ID | K (SEQ |
| | SC (SEQ | (SEQ ID | ID NO: | ID NO: | EDLGVYH | NO: | ID NO: |
| | ID NO: | NO: 6259) | 6282) | 6260) | C (SEQ ID | 6261) | 6284) |
| | 6281) | | | | NO: 6283) | | |
| Murine | NIVLTQS | RASESV | WYQQRP | LASNL | GVPARFSG | QQNNE | FGAG |
| anti- | PASLAVS | DSFGISF | GQPPKL | ES | SGSRTDFT | DPLT | TKLEL |
| calreticulin | LGQRATI | MH (SEQ | LIY (SEQ | (SEQ | LTIDPVEA | (SEQ ID | K (SEQ |
| antibody | SC (SEQ | ID NO: | ID NO: | ID NO: | DDAATYY | NO: 248) | ID NO: |
| 16B11.1 | ID NO: | 251) | 6375) | 246) | C (SEQ ID | | 249) |
| light chain | 6374) | | | | NO: 247) | | |
| variable | | | | | | | |
| region | | | | | | | |
| Humanized | DIVLTQT | RASESV | WYLQKP | LASNL | GVPDRFSG | QQNNE | FGQG |
| anti- | PLSLSVT | DSFGISF | GQSPQL | ES | SGSRTDFT | DPLT | TKLEI |
| calreticulin | PGQPASIS | MH (SEQ | LIY (SEQ | (SEQ | LKISRVEA | (SEQ ID | K (SEQ |
| light chain | C (SEQ ID | ID NO: | ID NO: | ID NO: | EDVGVYY | NO: 255) | ID NO: |
| variable | NO: 250) | 251) | 252) | 253) | C (SEQ ID | | 256) |
| region | | | | | NO: 254) | | |
| variant 1 | | | | | | | |
| Humanized | DIVLTQS | RASESV | WYQQRP | LASNL | GVPDRFSG | QQNNE | FGQG |
| anti- | PLSLPVT | DSFGISF | GQSPRL | ES | SGSRTDFT | DPLT | TKLEI |
| calreticulin | LGQPASI | MH (SEQ | LIY (SEQ | (SEQ | LKISRVEA | (SEQ ID | K (SEQ |
| light chain | SC (SEQ | ID NO: | ID NO: | ID NO: | EDVGVYY | NO: 262) | ID NO: |
| variable | ID NO: | 258) | 259) | 260) | C (SEQ ID | | 263) |
| region | 257) | | | | NO: 261) | | |
| variant 2 | | | | | | | |
| Humanized | DIVLTQT | RASESV | WYLQKP | LASNL | GVPDRFSG | QQNNE | FGQG |
| anti- | PLSLPVT | DSFGISF | GQSPQL | ES | SGSRTDFT | DPLT | TKLEI |
| calreticulin | PGEPASIS | MH (SEQ | LIY (SEQ | (SEQ | LKISRVEA | (SEQ ID | K (SEQ |
| light chain | C (SEQ ID | ID NO: | ID NO: | ID NO: | EDVGVYY | NO: 269) | ID NO: |
| variable | NO: 264) | 265) | 266) | 267) | C (SEQ ID | | 270) |
| region | | | | | NO: 268) | | |
| variant 3 | | | | | | | |
| Humanized | EIVLTQSP | RASESV | WYQQK | LASNL | GIPARFSG | QQNNE | FGQG |
| anti- | ATLSLSP | DSFGISF | PGQAPR | ES | SGSRTDFT | DPLT | TKLEI |
| calreticulin | GERATLS | MH (SEQ | LLIY | (SEQ | LTISSLEPE | (SEQ ID | K (SEQ |
| light chain | C (SEQ ID | ID NO: | (SEQ ID | ID NO: | DFAVYYC | NO: 276) | ID NO: |
| variable | NO: 271) | 272) | NO: 273) | 274) | (SEQ ID | | 277) |
| region | | | | | NO: 275) | | |
| variant 4 | | | | | | | |
| Humanized | DIQLTQS | RASESV | WYQQK | LASNL | GVPSRFSG | QQNNE | FGQG |
| anti- | PSSLSAS | DSFGISF | PGKAPK | ES | SGSRTDFT | DPLT | TKLEI |
| calreticulin | VGDRVTI | MH (SEQ | LLIY | (SEQ | FTISSLQPE | (SEQ ID | K (SEQ |
| light chain | TC (SEQ | ID NO: | (SEQ ID | ID NO: | DIATYYC | NO: 283) | ID NO: |
| variable | ID NO: | 279) | NO: 280) | 281) | (SEQ ID | | 284) |
| region | 278) | | | | NO: 282) | | |
| variant 5 | | | | | | | |
| 6C10 | EIVLTQSP | STSSSVT | WYQQK | STSNL | GVPTRFSG | QQCLSS | FGAG |
| Parental | ASKAASQ | TNYLH | PDTPPKL | AS | SGSGTSYS | PCT | TKLEI |
| VL BK052 | GEEVTIT | (SEQ ID | LIY (SEQ | (SEQ | LTISNMQG | (SEQ ID | K (SEQ |
| | C (SEQ ID | NO: 7387) | ID NO: | ID NO: | EDVATYY | NO: | ID NO: |
| | NO: 7386) | | 7388) | 7389) | C (SEQ ID | 7392) | 7393) |
| | | | | | NO: 7391) | | |
| 6C10 VL | EIVLTQSP | STSSSVT | WYQQK | STSNL | GVPTRFSG | QQSLSS | FGAG |
| BK210 - | ASKAASQ | TNYLH | PDTPPKL | AS | SGSGTSYS | PST | TKLEI |
| C91S/C96S | GEEVTIT | (SEQ ID | LIY (SEQ | (SEQ | LTISNMQG | (SEQ ID | K (SEQ |
| mutant | C (SEQ ID | NO: 7387) | ID NO: | ID NO: | EDVATYY | NO: | ID NO: |
| | NO: 7386) | | 7388) | 7389) | C (SEQ ID | 7410) | 7393) |
| | | | | | NO: 7391) | | |
| 6C10 VL | EIVLTQSP | STSSSVT | WYQQK | STSNL | GVPTRFSG | QQSLSS | FGAG |
| BK210 - | ASKAASQ | TNYLH | PDTPPKL | AS | SGSGTSYS | PCT | TKLEI |
| C91S | GEEVTIT | (SEQ ID | LIY (SEQ | (SEQ | LTISNMQG | (SEQ ID | K (SEQ |
| mutant | C (SEQ ID | NO: 7387) | ID NO: | ID NO | EDVATYY | NO | ID NO: |
| | NO: 7386) | | 7388) | 7389) | C (SEQ ID | 7415) | 7393) |
| | | | | | NO: 7391) | | |
| 6C10 VL | EIVLTQSP | STSSSVT | WYQQK | STSNL | GVPTRFSG | QQCLSS | FGAG |
| BK210 - | ASKAASQ | TNYLH | PDTPPKL | AS | SGSGTSYS | PST | TKLEI |
| C96S | GEEVTIT | (SEQ ID | LIY (SEQ | (SEQ | LTISNMQG | SEQ ID | K (SEQ |
| mutant | C (SEQ ID | NO: 7387) | ID NO: | ID NO: | EDVATYY | NO: | ID NO: |
| | NO: 7386) | | 7388) | 7389) | C (SEQ ID | 7417) | 7393) |
| | | | | | NO: 7391) | | |
| 6C10 | DIQLTQS | STSSSVT | WYQQK | STSNL | GVPSRFSG | QQCLSS | FGQG |
| humanized | PSFLSAS | TNYLH | PGKAPK | AS | SGSGTEYT | PCT | TKLEI |
| light chain | VGDRVTI | (SEQ ID | LLIY | (SEQ | LTISSLQPE | (SEQ ID | K (SEQ |
| variable | TC (SEQ | NO: 7387) | (SEQ ID | ID NO: | DFATYYC | NO: | ID NO: |
| region | ID NO: | | NO: 280) | 7389) | (SEQ ID | 7392) | 256) |
| variant 1 | 7426) | | | | NO: 7427) | | |
| (BK198) | | | | | | | |
| 6C10 | DIVLTQS | STSSSVT | WYQQK | STSNL | GVPDRFSG | QQCLSS | FGQG |
| humanized | PDSLAVS | TNYLH | PGQPPK | AS | SGSGTDYT | PCT | TKLEI |
| light chain | LGERATI | (SEQ ID | LLIY | (SEQ | LTISSLQAE | (SEQ ID | K (SEQ |
| variable | NC (SEQ | NO: 7387) | (SEQ ID | ID NO: | DVAVYYC | NO: | ID NO: |
| region | ID NO: | | NO: 7430) | 7389) | (SEQ ID | 7392) | 256) |
| variant 2 | 7429) | | | | NO: 7431) | | |
| (BK199) | | | | | | | |
| 6C10 | EIVLTQSP | STSSSVT | WYQQK | STSNL | GIPDRFSG | QQCLSS | FGQG |
| humanized | ATLSLSP | TNYLH | PGQAPK | AS | SGSGTDYT | PCT | TKLEI |
| light chain | GERATLS | (SEQ ID | LLIY | (SEQ | LTISRLEPE | (SEQ ID | K (SEQ |
| variable | C (SEQ ID | NO: 7387) | (SEQ ID | ID NO: | DFAVYYC | NO: | ID NO: |
| region | NO: 271) | | NO: 7433) | 7389) | (SEQ ID | 7392) | 256) |
| variant 3 | | | | | NO: 7434) | | |
| (BK200) | | | | | | | |
| 6C10_ | EIVLTQSP | STSSSVT | WYQQK | STSNL | GIPARFSG | QQCLSS | FGQG |
| hum2_scFv | ATLSLSP | TNYLH | PGQAPK | AS | SGSGTDYT | PCT | TKLEI |
| VL | GERATLS | (SEQ ID | LLIY | (SEQ | LTISSLQPE | (SEQ ID | K (SEQ |
| (BKM0162) | C (SEQ ID | NO: 7387) | (SEQ ID | ID NO: | DFAVYYC | NO: | ID NO: |
| | NO: 271) | | NO: 7433) | 7389) | (SEQ ID | 7392) | 256) |
| | | | | | NO: 7439) | | |
| 6C10_hum3_ | DIQLTQS | STSSSVT | WYQQK | STSNL | GVPSRFSG | QQSLSS | FGOG |
| scFv VL | PSFLSAS | TNYLH | PGKAPK | AS | SGSGTEYT | PST | TKLEI |
| (BKM0163) | VGDRVTI | (SEQ ID | LLIY | (SEQ | LTISSLQPE | (SEQ ID | K (SEQ |
| | TC (SEQ | NO: 7387) | (SEQ ID | ID NO: | DFATYYC | NO: | ID NO: |
| | ID NO: | | NO: 280) | 7389) | (SEQ ID | 7410) | 256) |
| | 7426) | | | | NO: 7427) | | |
| 6C10_hum4_ | EIVLTQSP | STSSSVT | WYQQK | STSNL | GIPARFSG | QQSLSS | FGQG |
| scFv VL | ATLSLSP | TNYLH | PGQAPK | AS | SGSGTDYT | PST | TKLEI |
| (BKM0164) | GERATLS | (SEQ ID | LLIY | (SEQ | LTISSLQPE | (SEQ ID | K (SEQ |
| | C (SEQ ID | NO: 7387) | (SEQ ID | ID NO: | DFAVYYC | NO: | ID NO: |
| | NO: 271) | | NO: 7433) | 7389) | (SEQ ID | 7410) | 256) |
| | | | | | NO: 7439) | | |

TABLE 6
Exemplary FWRs of calreticulin-targeting antigen binding

| SEQ ID NO | Description | Sequence |
|---|---|---|
| SEQ ID NO: | Ab-1 | X₁VQLX₂QSGX₃EX₄X_{5K}X₆GASVKX₇SCKASG, wherein: |
| 6224 | VHFWR1 | X₁ is not E, |
| | | X₂ is not E, |
| | | X₃ is not P, |
| | | X₄ is not L, |
| | | X₅ is not V, |
| | | X₆ is not T, or |
| | | X₇ is not I |
| SEQ ID NO: | Ab-1 | WVX₁QX₂X₃GX₄X₅LX₆WX₇G, wherein: |
| 6226 | VHFWR2 | X₁ is not K, |
| | | X₂ is not S, |
| | | X₃ is not H, |
| | | X₄ is not K, |
| | | X₅ is not S, |
| | | X₆ is not E, or |
| | | X₇ is not I |
| SEQ ID NO: | Ab-1 | X₁X₂TX₃TVDTSX₄STAYX₅X₆X₇X₈SLX₉SX₁₀DX₁₁AX₁₂YYCA, |
| 6228 | VHFWR3 | wherein: |
| | | X₁ is not K, |
| | | X₂ is not A, |
| | | X₃ is not F, |
| | | X₄ is not S, |
| | | X₅ is not M, |
| | | X₆ is not Q, |
| | | X₇ is not F, |
| | | X₈ is not N, |
| | | X₉ is not T, |
| | | X₁₀ is not G, |
| | | X₁₁ is not S, or |
| | | X₁₂ is not V |
| SEQ ID NO: | Ab-1 | WGQGTX₁VTVSS, wherein: |
| 6230 | VHFWR4 | X₁ is not S |
| SEQ ID NO: | Ab-2 | X₁VX₂LX₃ESGPX₄LVKX₅X₆X₇X₈LX₉LTCTVX₁₀G, wherein: |
| 6232 | VHFWR1 | X₁ is not D, |
| | | X₂ is not Q, |
| | | X₃ is not Q, |
| | | X₄ is not G, |
| | | X₅ is not N, |
| | | X₆ is not S, |
| | | X₇ is not Q, |
| | | X₈ is not S, |
| | | X₉ is not S, or |
| | | X₁₀ is not T |
| SEQ ID NO: | Ab-2 | WIRQX₁PGX₂X₃LEWX₄X₅, wherein: |
| 6234 | VHFWR2 | X₁ is not F, |
| | | X₂ is not N, |
| | | X₃ is not K, |
| | | X₄ is not M, or |
| | | X₅ is not G |
| SEQ ID NO: | Ab-2 | RX₁SITX₂DTSKX₃QX₄X₅LX₆X₇X₈X₉X₁₀X₁₁PX₁₂DTATYYCAR, |
| 6236 | VHFWR3 | wherein: |
| | | X₁ is not I, |
| | | X₂ is not R, |
| | | X₃ is not N, |
| | | X₄ is not F, |
| | | X₅ is not F, |
| | | X₆ is not Q, |
| | | X₇ is not L, |
| | | X₈ is not N, |
| | | X₉ is not S, |
| | | X₁₀ is not V, |
| | | X₁₁ is not T, or |
| | | X₁₂ is not E |
| SEQ ID NO: | Ab-2 | WGQGTX₁VTVSS, wherein: |
| 6230 | VHFWR4 | X₁ is not S |
| SEQ ID NO: | Ab-1/2 | DVVMTQX₁PLX₂LX₃VTX₄GQPASISC, wherein: |
| 6238 | VLFWR1 | X₁ is not T, |
| | | X₂ is not T, |
| | | X₃ is not S, or |
| | | X₄ is not I |
| SEQ ID NO: | Ab-1/2 | WLX₁QRPGQSPX₂RLIY, wherein: |
| 6240 | VLFWR2 | X₁ is not L, or |
| | | X₂ is not K |
| SEQ ID NO: | Ab-1/2 | GVPDRFX₁GSGSGTDFTLKISRVEAEDX₂GVYHC, wherein: |
| 6242 | VLFWR3 | X₁ is not T, or |
| | | X₂ is not L |
| SEQ ID NO: | Ab-1/2 | FGGGTKX₁EIK, wherein: |
| 6244 | VLFWR4 | X₁ is not L |

In some embodiments, the calreticulin-targeting antigen binding domain comprises a VH comprising a VH amino acid sequence as listed in Table 24, or an amino acid sequence having at least 75%, 80%, 85%, 90%, 95%, 96%, 97%, 98%, or 99% sequence identity thereto. In some embodiments, the calreticulin-targeting antigen binding domain comprises a VL comprising a VL amino acid sequence as listed in Table 24, or an amino acid sequence having at least 75%, 80%, 85%, 90%, 95%, 96%, 97%, 9800, or 9900 sequence identity thereto. In some embodiments, the calreticulin-targeting antigen binding domain comprises: (i) a VH comprising a VH amino acid sequence as listed in Table 24, or an amino acid sequence having at least 75%, 80%, 85%, 90%, 95%, 96%, 97%, 98%, or 99% sequence identity thereto, and (ii) a VL comprising a VL amino acid sequence as listed in Table 24, or an amino acid sequence having at least 75%, 80%, 85%, 90%, 95%, 96%, 97%, 98%, or 99% sequence identity thereto.

TABLE 24
Exemplary variable regions of calreticulin-targeting antigen binding (underlining
indicates CDR sequences)

| SEQ | | |
|---|---|---|
| ID NO | Description | Sequence |
| SEQ | AbH-1 heavy | QVQLVQSGAEVKKPGASVKVSCKASGYSFTGYYIHWVRQAPG |
| ID NO: | chain variable | QELGWMGYISCYNGASSYNQKFKGRVTMTVDTSISTAYTELSSL |
| 6247 | region | RSEDTATYYCA SSMDYWGQGTLVTVSS |
| SEQ | AbH-2 heavy | QVTLKESGPVLVKPTETLTLTCTVSGYSITSDYAWNWIRQPPGK |
| ID NO: | chain variable | ALEWLAYISYSGSTSYNPSLKSRLSITKDTSKSQVVLTMTNMDP |
| 6248 | region | VDTATYYCARDPPYYYGSWGQGTTVTVSS |
| SEQ | AbH-1 / AbH-2 | DVVMTQSPLSLPVTLGQPASISCKSSQSLLDSDGKTYLNWLQQR |
| ID NO: | light chain | PGQSPRRLIYLVSKLDSGVPDRFSGSGSGTDFTLKISRVEAEDVG |
| 6249 | variable region | VYHCWQGTHFPYTFGGGTKVEIK |
| SEQ | AbM-1 heavy | EVQLEQSGPELVKTGASVKISCKASGYSFTGYYIHWVKQSHGKS |
| ID NO: | chain variable | LEWIGYISCYNGASSYNQKFKGKATFTVDTSSSTAYMQFNSLTS |
| 6250 | region | GDSAVYYCA SSMDYWGQGTSVTVSS |
| SEQ | AbM-2 heavy | DVQLQESGPGLVKNSQSLSLTCTVTGYSITSDYAWNWIRQFPGN |
| ID NO: | chain variable | KLEWMGYISYSGSTSYNPSLKSRISITRDTSKNQFFLQLNSVTPED |
| 6251 | region | TATYYCARDPPYYYGSNGTWGQGTSVTVSS |
| SEQ | AbM-1 / AbM-2 | DVVMTQTPLTLSVTIGQPASISCKSSQSLLDSDGKTYLNWLLQRP |
| ID NO: | light chain | GQSPKRLIYLVSKLDSGVPDRFTGSGSGTDFTLKISRVEAEDLGV |
| 6252 | variable region | YHCWQGTHFPYTFGGGTKLEIK |
| SEQ | Murine anti- | EVKLVESGGGLVKPGGSLKLSCAASGFAFSRYDMSWVRQTPEK |
| ID NO: | calreticulin | RLEWVATISSGGSYTYYPDSVKGRFTISRDNARNTLYLQMSSLR |
| 6347 | antibody | SEDTALYYCARHSAYYVNYENAMDYWGQGTSVTVSS |
| | 16B11.1 heavy | |
| | chain variable | |
| | region | |
| SEQ | 6C10 Parental | EVQLVEKGGGLVQPGKSLKLSCTASGFTFSEYWMNWLRQAPG |
| ID NO: | VH BK051 | KGLEWVGVIKYKYSNYATEFAESVKGRFTISRDDSKSSVYLQMT |
| 7418 | | NLRAEDTAIYYCARGRDVQDYWGQGTMVTVSS |
| SEQ | 6C10 | EVQLVESGGGLVQPGPSLRLSCTASGFTFSEYWMNWLRQAPGK |
| ID NO: | humanized | GLEWVGVIKYKYSNYATEFAESVKGRFTISRDDSKSIVYLQMNS |
| 7425 | heavy chain | LKTEDTAVYYCARGRDVQDYWGQGTMVTVSS |
| | variable region | |
| | variant 1 | |
| | (BK197) | |
| SEQ | 6C10_hum1_sc | EVQLVESGGGLVQPGRSLRLSCTASGFTFSEYWMNWLRQAPGK |
| ID NO: | Fv VH | GLEWVGVIKYKYSNYATEFAESVKGRFTISRDDSKSIVYLQMNS |
| 7438 | (BKM0161) | LKTEDTAVYYCARGRDVQDYWGQGTMVTVSS |
| SEQ | 6C10_hum5_sc | EVQLVESGGGLVQPGGSLRLSCAASGFTFSEYWMNWLRQAPGK |
| ID NO: | Fv VH | GLEWVGVIKYKYSNYATEFAESVKGRFTISRDDSKSSVYLQMNS |
| 7446 | (BKM0165) | LKTEDTAVYYCARGRDVQDYWGQGTMVTVSS |
| SEQ | Murine anti- | NIVLTQSPASLAVSLGQRATISCRASESVDSFGISFMHWYQQRPG |
| ID NO: | calreticulin | QPPKLLIYLASNLESGVPARFSGSGSRTDFTLTIDPVEADDAATY |
| 6348 | antibody | YCQQNNEDPLTFGAGTKLELK |
| | 16B11.1 light | |
| | chain variable | |
| | region | |
| SEQ | Humanized anti- | QVQLVESGGGLVKPGGSLRLSCAASGFAFSRYDMSWIRQAPGK |
| ID NO: | calreticulin | GLEWVATISSGGSYTYYPDSVKGRFTISRDNAKNSLYLQMNSLR |
| 6349 | heavy chain | AEDTAVYYCARHSAYYVNYENAMDYWGQGTLVTVSS |
| | variable region | |
| | variant 1 | |
| SEQ | Humanized anti- | QVQLVESGGGVVQPGRSLRLSCAASGFAFSRYDMSWVRQAPGK |
| ID NO: | calreticulin | GLEWVATISSGGSYTYYPDSVKGRFTISRDNSKNTLYLQMNSLR |
| 6350 | heavy chain | AEDTAVYYCARHSAYYVNYENAMDYWGQGTLVTVSS |
| | variable region | |
| | variant 2 | |
| SEQ | Humanized anti- | EVQLVESGGGLVQPGGSLRLSCAASGFAFSRYDMSWVRQAPGK |
| ID NO: | calreticulin | GLEWVATISSGGSYTYYPDSVKGRFTISRDNSKNTLYLQMNSLR |
| 6351 | heavy chain | AEDTAVYYCARHSAYYVNYENAMDYWGQGTLVTVSS |
| | variable region | |
| | variant 3 | |
| SEQ | Humanized anti- | DIVLTQTPLSLSVTPGQPASISCRASESVDSFGISFMHWYLQKPG |
| ID NO: | calreticulin light | QSPQLLIYLASNLESGVPDRFSGSGSRTDFTLKISRVEAEDVGVY |
| 6352 | chain variable | YCQQNNEDPLTFGQGTKLEIK |
| | region variant 1 | |
| SEQ | Humanized anti- | DIVLTQSPLSLPVTLGQPASISCRASESVDSFGISFMHWYQQRPG |
| ID NO: | calreticulin light | QSPRLLIYLASNLESGVPDRFSGSGSRTDFTLKISRVEAEDVGVY |
| 6353 | chain variable | YCQQNNEDPLTFGQGTKLEIK |
| | region variant 2 | |
| SEQ | Humanized anti- | DIVLTQTPLSLPVTPGEPASISCRASESVDSFGISFMHWYLQKPGQ |
| ID NO: | calreticulin light | SPQLLIYLASNLESGVPDRFSGSGSRTDFTLKISRVEAEDVGVYY |
| 6354 | chain variable | CQQNNEDPLTFGQGTKLEIK |
| | region variant 3 | |
| SEQ | Humanized anti- | EIVLTQSPATLSLSPGERATLSCRASESVDSFGISFMHWYQQKPG |
| ID NO: | calreticulin light | QAPRLLIYLASNLESGIPARFSGSGSRTDFTLTISSLEPEDFAVYYC |
| 6355 | chain variable | QQNNEDPLTFGQGTKLEIK |
| | region variant 4 | |
| SEQ | Humanized anti- | DIQLTQSPSSLSASVGDRVTITCRASESVDSFGISFMHWYQQKPG |
| ID NO: | calreticulin light | KAPKLLIYLASNLESGVPSRFSGSGSRTDFTFTISSLQPEDIATYYC |
| 6356 | chain variable | QQNNEDPLTFGQGTKLEIK |
| | region variant 5 | |
| SEQ | 6C10 Parental | EIVLTQSPASKAASQGEEVTITCSTSSSVTTNYLHWYQQKPDTPP |
| ID NO: | VL BK052 | KLLIYSTSNLASGVPTRFSGSGSGTSYSLTISNMQGEDVATYYCQ |
| 7419 | | QCLSSPCTFGAGTKLEIK |
| SEQ | 6C10 VL | EIVLTQSPASKAASQGEEVTITCSTSSSVTTNYLHWYQQKPDTPP |
| ID NO: | BK210 - | KLLIYSTSNLASGVPTRFSGSGSGTSYSLTISNMQGEDVATYYCQ |
| 7420 | C91S/C96S | QSLSSPSTFGAGTKLEIK |
| | mutant | |
| SEQ | 6C10 VL | EIVLTQSPASKAASQGEEVTITCSTSSSVTTNYLHWYQQKPDTPP |
| ID NO: | BK210 - C91S | KLLIYSTSNLASGVPTRFSGSGSGTSYSLTISNMQGEDVATYYCQ |
| 7421 | mutant | QSLSSPCTFGAGTKLEIK |
| SEQ | 6C10 VL | EIVLTQSPASKAASQGEEVTITCSTSSSVTTNYLHWYQQKPDTPP |
| ID NO: | BK210 - C96S | KLLIYSTSNLASGVPTRFSGSGSGTSYSLTISNMQGEDVATYYCQ |
| 7422 | mutant | QCLSSPSTFGAGTKLEIK |
| SEQ | 6C10 | DIQLTQSPSFLSASVGDRVTITCSTSSSVTTNYLHWYQQKPGKAP |
| ID NO: | humanized light | KLLIYSTSNLASGVPSRFSGSGSGTEYTLTISSLQPEDFATYYCQQ |
| 7428 | chain variable | CLSSPCTFGQGTKLEIK |
| | region variant 1 | |
| | (BK198) | |
| SEQ | 6C10 | DIVLTQSPDSLAVSLGERATINCSTSSSVTTNYLHWYQQKPGQPP |
| ID NO: | humanized light | KLLIYSTSNLASGVPDRFSGSGSGTDYTLTISSLQAEDVAVYYCQ |
| 7432 | chain variable | QCLSSPCTFGQGTKLEIK |
| | region variant 2 | |
| | (BK199) | |
| SEQ | 6C10 | EIVLTQSPATLSLSPGERATLSCSTSSSVTTNYLHWYQQKPGQAP |
| ID NO: | humanized light | KLLIYSTSNLASGIPDRFSGSGSGTDYTLTISRLEPEDFAVYYCQQ |
| 7435 | chain variable | CLSSPCTFGQGTKLEIK |
| | region variant 3 | |
| | (BK200) | |
| SEQ | 6C10_ | EIVLTQSPATLSLSPGERATLSCSTSSSVTTNYLHWYQQKPGQAP |
| ID NO: | hum2_scFv VL | KLLIYSTSNLASGIPARFSGSGSGTDYTLTISSLQPEDFAVYYCQQ |
| 7440 | (BKM0162) | CLSSPCTFGQGTKLEIK |
| SEQ | 6C10_hum3_sc | DIQLTQSPSFLSASVGDRVTITCSTSSSVTTNYLHWYQQKPGKAP |
| ID NO: | Fv VL | KLLIYSTSNLASGVPSRFSGSGSGTEYTLTISSLQPEDFATYYCQQ |
| 7441 | (BKM0163) | SLSSPSTFGQGTKLEIK |
| SEQ | 6C10_hum4_sc | EIVLTQSPATLSLSPGERATLSCSTSSSVTTNYLHWYQQKPGQAP |
| ID NO: | Fv VL | KLLIYSTSNLASGIPARFSGSGSGTDYTLTISSLQPEDFAVYYCQQ |
| 7442 | (BKM0164) | SLSSPSTFGQGTKLEIK |

In some embodiments, the calreticulin-targeting antigen binding domain comprises an scFv comprising an amino acid sequence as listed in Table 25, or an amino acid sequence having at least 750%, 80%, 85%, 90%, 950%, 960%, 97%, 980%, or 99% sequence identity thereto. In some embodiments, the calreticulin-targeting antigen binding domain comprises an scFv comprising a VH amino acid sequence as listed in Table 25, or an amino acid sequence having at least 75%, 80%, 850%, 90%, 95%, 960%, 97%, 98%, or 99% sequence identity thereto. In some embodiments, the calreticulin-targeting antigen binding domain comprises an scFv comprising a VL amino acid sequence as listed in Table 25, or an amino acid sequence having at least 750%, 80%, 85%, 90%, 95%, 96%, 97%, 98%, or 99% sequence identity thereto. In some embodiments, the calreticulin-targeting antigen binding domain comprises an scFv comprising a spacer amino acid sequence as listed in Table 25, or an amino acid sequence having at least 75%, 80%, 85%, 90%, 95%, 96%, 97%, 98%, or 99% sequence identity thereto.

TABLE 25
Exemplary scFv sequences for calreticulin-targeting antigen binding (underlining
indicates CDR sequences)

| SEQ ID | | |
|---|---|---|
| NO: | Description | Sequence |
| 7499 | 6C10_hum1_ | EVQLVESGGGLVQPGRSLRLSCTASGFTFSEYWMNWLRQAPGKG |
| | scFv | LEWVGVIKYKYSNYATEFAESVKGRFTISRDDSKSIVYLQMNSLK |
| | (BKM0161) | TEDTAVYYCARGRDVQDYWGQGTMVTVSSGGGGSGGGGSGGG |
| | | GSGGGGSDIQLTQSPSFLSASVGDRVTITCSTSSSVTTNYLHWYQQ |
| | | KPGKAPKLLIYSTSNLASGVPSRFSGSGSGTEYTLTISSLQPEDFAT |
| | | YYCQQCLSSPCTFGQGTKLEIK |
| 7500 | 6C10_ | EVQLVESGGGLVQPGRSLRLSCTASGFTFSEYWMNWLRQAPGKG |
| | hum2_scFv | LEWVGVIKYKYSNYATEFAESVKGRFTISRDDSKSIVYLQMNSLK |
| | (BKM0162) | TEDTAVYYCARGRDVQDYWGQGTMVTVSSGGGGSGGGGSGGG |
| | | GSGGGGSEIVLTQSPATLSLSPGERATLSCSTSSSVTTNYLHWYQQ |
| | | KPGQAPKLLIYSTSNLASGIPARFSGSGSGTDYTLTISSLQPEDFAV |
| | | YYCQQCLSSPCTFGQGTKLEIK |
| 7501 | 6C10_hum3_ | EVQLVESGGGLVQPGRSLRLSCTASGFTFSEYWMNWLRQAPGKG |
| | scFv | LEWVGVIKYKYSNYATEFAESVKGRFTISRDDSKSIVYLQMNSLK |
| | (BKM0163) | TEDTAVYYCARGRDVQDYWGQGTMVTVSSGGGGSGGGGSGGG |
| | | GSGGGGSDIQLTQSPSFLSASVGDRVTITCSTSSSVTTNYLHWYQQ |
| | | KPGKAPKLLIYSTSNLASGVPSRFSGSGSGTEYTLTISSLQPEDFAT |
| | | YYCQQSLSSPSTFGQGTKLEIK |
| 7502 | 6C10_hum4_ | EVQLVESGGGLVQPGRSLRLSCTASGFTFSEYWMNWLRQAPGKG |
| | scFv | LEWVGVIKYKYSNYATEFAESVKGRFTISRDDSKSIVYLQMNSLK |
| | (BKM0164) | TEDTAVYYCARGRDVQDYWGQGTMVTVSSGGGGSGGGGSGGG |
| | | GSGGGGSEIVLTQSPATLSLSPGERATLSCSTSSSVTTNYLHWYQQ |
| | | KPGQAPKLLIYSTSNLASGIPARFSGSGSGTDYTLTISSLQPEDFAV |
| | | YYCQQSLSSPSTFGQGTKLEIK |
| 7503 | 6C10_hum5_ | EVQLVESGGGLVQPGGSLRLSCAASGFTFSEYWMNWLRQAPGKG |
| | scFv | LEWVGVIKYKYSNYATEFAESVKGRFTISRDDSKSSVYLQMNSLK |
| | (BKM0165) | TEDTAVYYCARGRDVQDYWGQGTMVTVSSGGGGSGGGGSGGG |
| | | GSGGGGSDIQLTQSPSFLSASVGDRVTITCSTSSSVTTNYLHWYQQ |
| | | KPGKAPKLLIYSTSNLASGVPSRFSGSGSGTEYTLTISSLQPEDFAT |
| | | YYCQQCLSSPCTFGQGTKLEIK |
| 7504 | 6C10_hum6_ | EVQLVESGGGLVQPGGSLRLSCAASGFTFSEYWMNWLRQAPGKG |
| | scFv | LEWVGVIKYKYSNYATEFAESVKGRFTISRDDSKSSVYLQMNSLK |
| | (BKM0166) | TEDTAVYYCARGRDVQDYWGQGTMVTVSSGGGGSGGGGSGGG |
| | | GSGGGGSEIVLTQSPATLSLSPGERATLSCSTSSSVTTNYLHWYQQ |
| | | KPGQAPKLLIYSTSNLASGIPARFSGSGSGTDYTLTISSLQPEDFAV |
| | | YYCQQCLSSPCTFGQGTKLEIK |
| 7505 | 6C10_hum7_ | EVQLVESGGGLVQPGGSLRLSCAASGFTFSEYWMNWLRQAPGKG |
| | scFv | LEWVGVIKYKYSNYATEFAESVKGRFTISRDDSKSSVYLQMNSLK |
| | (BKM0167) | TEDTAVYYCARGRDVQDYWGQGTMVTVSSGGGGSGGGGSGGG |
| | | GSGGGGSDIQLTQSPSFLSASVGDRVTITCSTSSSVTTNYLHWYQQ |
| | | KPGKAPKLLIYSTSNLASGVPSRFSGSGSGTEYTLTISSLQPEDFAT |
| | | YYCQQSLSSPSTFGQGTKLEIK |
| 7506 | 6C10_hum8_ | EVQLVESGGGLVQPGGSLRLSCAASGFTFSEYWMNWLRQAPGKG |
| | scFv | LEWVGVIKYKYSNYATEFAESVKGRFTISRDDSKSSVYLQMNSLK |
| | (BKM0168) | TEDTAVYYCARGRDVQDYWGQGTMVTVSSGGGGSGGGGSGGG |
| | | GSGGGGSEIVLTQSPATLSLSPGERATLSCSTSSSVTTNYLHWYQQ |
| | | KPGQAPKLLIYSTSNLASGIPARFSGSGSGTDYTLTISSLQPEDFAV |
| | | YYCQQSLSSPSTFGQGTKLEIK |

In some embodiments, the calreticulin-targeting antigen binding domain comprises an Fc region. In some embodiments, the Fc region is chosen from, e.g., the heavy chain constant regions of IgG1, IgG2, IgG3, IgG4, IgM, IgA1, IgA2, IgD, and IgE. In some embodiments, the Fc region is chosen from the heavy chain constant regions of IgG1, IgG2, IgG3, and IgG4. In some embodiments, the Fc region is chosen from the heavy chain constant region of IgG1 or IgG2 (e.g., human IgG1, or IgG2). In some embodiments, the heavy chain constant region is human IgG2a. In some embodiment, the heavy chain constant region comprises a murine IgG2a sequence, e.g., SEQ ID NO: 7448 below, or an amino acid sequence having at least 75%, 80%, 85%, 90%, 95%, 96%, 97%, 98%, or 99% sequence identity thereto:

(SEQ ID NO: 7448)
AKTTAPSVYPLAPVCGDTTGSSVTLGCLVKGYFPEPVTLTWNSGSLSSGV
HTFPAVLQSDLYTLSSSVTVTSSTWPSQSITCNVAHPASSTKVDKKIEPR
GPTIKPCPPCKCPAPNLLGGPSVFIFPPKIKDVLMISLSPIVTCVVVDVS
EDDPDVQISWFVNNVEVHTAQTQTHREDYNSTLRVVSALPIQHQDWMSGK
EFKCKVNNKDLPAPIERTISKPKGSVRAPQVYVLPPPEEEMTKKQVTLTC
MVTDFMPEDIYVEWTNNGKTELNYKNTEPVLDSDGSYFMYSKLRVEKKNW
VERNSYSCSVVHEGLHNHHTTKSFSRTPGK

In some embodiments, the Fc region comprises a Fc region variant, e.g., as described herein. In some embodiments, the Fc region comprises one or more mutations. In some embodiments, the Fc region comprises an LALAPG mutation. In some embodiments, the Fc region comprises the amino acid sequence of a murine IgG2a-LALAPG variant, e.g., the sequence of SEQ ID NOs: 7449 or 7450 below, or an amino acid sequence having at least 75%, 80%, 85%, 90%, 95%, 96%, 97%, 98%, or 99% sequence identity thereto:

(SEQ ID NO: 7449)
AKTTAPSVYPLAPVCGDTTGSSVTLGCLVKGYFPEPVTLTWNSGSLSSGV
HTFPAVLQSDLYTLSSSVTVTSSTWPSQSITCNVAHPASSTKVDKKIEPR
GPTIKPCPPCKCPAPNAAGGPSVFIFPPKIKDVLMISLSPIVTCVVVDVS
EDDPDVQISWFVNNVEVHTAQTQTHREDYNSTLRVVSALPIQHQDWMSGK
EFKCKVNNKDLGAPIERTISKPKGSVRAPQVYVLPPPEEEMTKKQVTLTC
MVTDFMPEDIYVEWTNNGKTELNYKNTEPVLDSDGSYFMYSKLRVEKKNW
VERNSYSCSVVHEGLHNHHTTKSFSRTPGK
(SEQ ID NO: 7450)
AKTTAPSVYPLAPVCGDTTGSSVTLGCLVKGYFPEPVTLTWNSGSLSSGV
HTFPAVLQSDLYTLSSSVTVTSSTWPSQSITCNVAHPASSTKVDKKIEPR
GPTIKPCPPCKCPAPNAAGGPSVFIFPPKIKDVLMISLSPIVTCVVVDVS
EDDPDVQISWFVNNVEVHTAQTQTHREDYNSTLRVVSALPIQHQDWMSGK
EFKCKVNNKDLGAPIERTISKPKGSVRAPQVYVLPPCEEEMTKKQVTLWC
MVTDFMPEDIYVEWTNNGKTELNYKNTEPVLDSDGSYFMYSKLRVEKKNW
VERNSYSCSVVHEGLHNHHTTKSFSRTPGK

In some embodiments, the Fc region comprises the amino acid sequence of a human IgG2a N2976A variant, e.g., the sequence of SEQ ID NO: 7453 below, or an amino acid sequence having at least 75%, 80%, 85%, 90%, 95%, 96%, 97%, 98%, or 99% sequence identity thereto:

(SEQ ID NO: 7453)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSCDKTHTCPPCPAPELLGGPSVFLFPPKPKDTLMISRTPEVTCVVVDVS
HEDPEVKFNWYVDGVEVHNAKTKPREEQYGSTYRVVSVLTVLHQDWLNGK
EYKCKVSNKALPAPIEKTISKAKGQPREPQVYTLPPSREEMTKNQVSLTC
LVKGFYPSDIAVEWESNGQPENNYKTTPPVLDSDGSFFLYSKLTVDKSRW
QQGNVFSCSVMHEALHNHYTQKSLSLSPGK

In some embodiments, the calreticulin-targeting antigen binding domain comprises a light chain constant region, e.g., a CL kappa region, e.g., a human CL kappa region. In some embodiments, the light chain constant region comprises the amino acid sequence of SEQ ID NO: 7451 below, or an amino acid sequence having at least 75%, 80%, 85%, 90%, 95%, 96%, 97%, 98%, or 99% sequence identity thereto:

(SEQ ID NO: 7451)
RADAAPTVSIFPPSSEQLTSGGASVVCFLNNFYPKDINVKWKIDGSERQ
NGVLNSWTDQDSKDSTYSMSSTLTLTKDEYERHNSYTCEATHKTSTSPI
VKSFNRNEC

In some embodiments, the light chain constant region comprises the amino acid sequence of SEQ ID NO: 7454 below, or an amino acid sequence having at least 75%, 80%, 85%, 90%, 95%, 96%, 97%, 98%, or 99% sequence identity thereto:

(SEQ ID NO: 7454)
RTVAAPSVFIFPPSDEQLKSGTASVVCLLNNFYPREAKVQWKVDNALQS
GNSQESVTEQDSKDSTYSLSSTLTLSKADYEKHKVYACEVTHQGLSSPV
TKSFNRGEC

### Additional Calreticulin-Targeting Antigen Binding Domains

In some embodiments, the calreticulin-targeting antigen binding domain comprises any CDR amino acid sequence or variable region amino acid sequence disclosed in Tables 16-19. In some embodiments, the calreticulin-targeting antigen binding domain comprises a VH comprising a heavy chain complementarity determining region 1 (VHCDR1) amino acid sequence of SEQ ID NO: 6253 (or a sequence with no more than 1, 2, 3, or 4 mutations, e.g., substitutions, additions, or deletions), a VHCDR2 amino acid sequence of SEQ ID NO: 243 (or a sequence with no more than 1, 2, 3, or 4 mutations, e.g., substitutions, additions, or deletions), and/or a VHCDR3 amino acid sequence of SEQ ID NO: 6255 (or a sequence with no more than 1, 2, 3, or 4 mutations, e.g., substitutions, additions, or deletions). In some embodiments, the calreticulin-targeting antigen binding domain comprises a VH comprising a VHCDR1 amino acid sequence of SEQ ID NO: 6253, a VHCDR2 amino acid sequence of SEQ ID NO: 243, and/or a VHCDR3 amino acid sequence of SEQ ID NO: 6255. In some embodiments, the calreticulin-targeting antigen binding domain comprises a VL comprising a light chain complementarity determining region 1 (VLCDR1) amino acid sequence of SEQ ID NO: 6259 (or a sequence with no more than 1, 2, 3, or 4 mutations, e.g., substitutions, additions, or deletions), a VLCDR2 amino acid sequence of SEQ ID NO: 6260 (or a sequence with no more than 1, 2, 3, or 4 mutations, e.g., substitutions, additions, or deletions), and/or a VLCDR3 amino acid sequence of SEQ ID NO: 6261 (or a sequence with no more than 1, 2, 3, or 4 mutations, e.g., substitutions, additions, or deletions). In some embodiments, the calreticulin-targeting antigen binding domain comprises a VL comprising a VLCDR1 amino acid sequence of SEQ ID NO: 6259, a VLCDR2 amino acid sequence of SEQ ID NO: 6260, and a VLCDR3 amino acid sequence of SEQ ID NO: 6261. In some embodiments, the calreticulin-targeting antigen binding domain comprises a VH comprising the amino acid sequence of SEQ ID NO: 244 (or an amino acid sequence having at least about 77%, 80%, 85%, 90%, 95%, or 99% sequence identity thereto). In some embodiments, the calreticulin-targeting antigen binding domain comprises a VL comprising the amino acid sequence of SEQ ID NO: 245 (or an amino acid sequence having at least about 93%, 95%, or 99% sequence identity thereto). In some embodiments, the calreticulin-targeting antigen binding domain comprises a VH comprising the amino acid sequence of SEQ ID NO: 244 and/or a VL comprising the amino acid sequence of SEQ ID NO: 245. In some embodiments, the calreticulin-targeting antigen binding domain comprises a VH comprising the amino acid sequence of SEQ ID NO: 6372, 234, 235, 236, or 237, or an amino acid sequence having at least about 80%, 85%, 90%, 95%, or 99% sequence identity thereto. In some embodiments, the calreticulin-targeting antigen binding domain comprises a VL comprising the amino acid sequence of SEQ ID NO: 238, 239, 240, 241, or 242, or an amino acid sequence having at least about 80%, 85%, 90%, 95%, or 99% sequence identity thereto. In some embodiments, the calreticulin-targeting antigen binding domain comprises a VH comprising the amino acid sequence of SEQ ID NO: 6372 (or an amino acid sequence having at least about 80%, 85%, 90%, 95%, or 99% sequence identity thereto) and a VL comprising the amino acid sequence of SEQ ID NO: 238 (or an amino acid sequence having at least about 80%, 85%, 90%, 95%, or 99% sequence identity thereto). In some embodiments, the calreticulin-targeting antigen binding domain comprises a VH comprising the amino acid sequence of SEQ ID NO: 6372 and a VL comprising the amino acid sequence of SEQ ID NO: 238. In some embodiments, the calreticulin-targeting antigen binding domain comprises a VH comprising the amino acid sequence of SEQ ID NO: 234 (or an amino acid sequence having at least about 80%, 85%, 90%, 95%, or 99% sequence identity thereto) and a VL comprising the amino acid sequence of SEQ ID NO: 238 (or an amino acid sequence having at least about 80%, 85%, 90%, 95%, or 99% sequence identity thereto). In some embodiments, the calreticulin-targeting antigen binding domain comprises a VH comprising the amino acid sequence of SEQ ID NO: 234 and a VL comprising the amino acid sequence of SEQ ID NO: 238. In some embodiments, the calreticulin-targeting antigen binding domain comprises a VH comprising the amino acid sequence of SEQ ID NO: 235 (or an amino acid sequence having at least about 80%, 85%, 90%, 95%, or 99% sequence identity thereto) and a VL comprising the amino acid sequence of SEQ ID NO: 238 (or an amino acid sequence having at least about 80%, 85%, 90%, 95%, or 99% sequence identity thereto). In some embodiments, the calreticulin-targeting antigen binding domain comprises a VH comprising the amino acid sequence of SEQ ID NO: 235 and a VL comprising the amino acid sequence of SEQ ID NO: 238. In some embodiments, the calreticulin-targeting antigen binding domain comprises a VH comprising the amino acid sequence of SEQ ID NO: 236 (or an amino acid sequence having at least about 80%, 85%, 90%, 95%, or 99% sequence identity thereto) and a VL comprising the amino acid sequence of SEQ ID NO: 238 (or an amino acid sequence having at least about 80%, 85%, 90%, 95%, or 99% sequence identity thereto). In some embodiments, the calreticulin-targeting antigen binding domain comprises a VH comprising the amino acid sequence of SEQ ID NO: 236 and a VL comprising the amino acid sequence of SEQ ID NO: 238. In some embodiments, the calreticulin-targeting antigen binding domain comprises a VH comprising the amino acid sequence of SEQ ID NO: 237 (or an amino acid sequence having at least about 80%, 85%, 90%, 95%, or 99% sequence identity thereto) and a VL comprising the amino acid sequence of SEQ ID NO: 238 (or an amino acid sequence having at least about 80%, 85%, 90%, 95%, or 99% sequence identity thereto). In some embodiments, the calreticulin-targeting antigen binding domain comprises a VH comprising the amino acid sequence of SEQ ID NO: 237 and a VL comprising the amino acid sequence of SEQ ID NO: 238. In some embodiments, the calreticulin-targeting antigen binding domain comprises a VH comprising the amino acid sequence of SEQ ID NO: 6372 (or an amino acid sequence having at least about 80%, 85%, 90%, 95%, or 99% sequence identity thereto) and a VL comprising the amino acid sequence of SEQ ID NO: 239 (or an amino acid sequence having at least about 80%, 85%, 90%, 95%, or 99% sequence identity thereto). In some embodiments, the calreticulin-targeting antigen binding domain comprises a VH comprising the amino acid sequence of SEQ ID NO: 6372 and a VL comprising the amino acid sequence of SEQ ID NO: 239. In some embodiments, the calreticulin-targeting antigen binding domain comprises a VH comprising the amino acid sequence of SEQ ID NO: 234 (or an amino acid sequence having at least about 80%, 85%, 90%, 95%, or 99% sequence identity thereto) and a VL comprising the amino acid sequence of SEQ ID NO: 239 (or an amino acid sequence having at least about 80%, 85%, 90%, 95%, or 99% sequence identity thereto). In some embodiments, the calreticulin-targeting antigen binding domain comprises a VH comprising the amino acid sequence of SEQ ID NO: 234 and a VL comprising the amino acid sequence of SEQ ID NO: 239. In some embodiments, the calreticulin-targeting antigen binding domain comprises a VH comprising the amino acid sequence of SEQ ID NO: 235 (or an amino acid sequence having at least about 80%, 85%, 90%, 95%, or 99% sequence identity thereto) and a VL comprising the amino acid sequence of SEQ ID NO: 239 (or an amino acid sequence having at least about 80%, 85%, 90%, 95%, or 99% sequence identity thereto). In some embodiments, the calreticulin-targeting antigen binding domain comprises a VH comprising the amino acid sequence of SEQ ID NO: 235 and a VL comprising the amino acid sequence of SEQ ID NO: 239. In some embodiments, the calreticulin-targeting antigen binding domain comprises a VH comprising the amino acid sequence of SEQ ID NO: 236 (or an amino acid sequence having at least about 80%, 85%, 90%, 95%, or 99% sequence identity thereto) and a VL comprising the amino acid sequence of SEQ ID NO: 239 (or an amino acid sequence having at least about 80%, 85%, 90%, 95%, or 99% sequence identity thereto). In some embodiments, the calreticulin-targeting antigen binding domain comprises a VH comprising the amino acid sequence of SEQ ID NO: 236 and a VL comprising the amino acid sequence of SEQ ID NO: 239. In some embodiments, the calreticulin-targeting antigen binding domain comprises a VH comprising the amino acid sequence of SEQ ID NO: 237 (or an amino acid sequence having at least about 80%, 85%, 90%, 95%, or 99% sequence identity thereto) and a VL comprising the amino acid sequence of SEQ ID NO: 239 (or an amino acid sequence having at least about 80%, 85%, 90%, 95%, or 99% sequence identity thereto). In some embodiments, the calreticulin-targeting antigen binding domain comprises a VH comprising the amino acid sequence of SEQ ID NO: 237 and a VL comprising the amino acid sequence of SEQ ID NO: 239. In some embodiments, the calreticulin-targeting antigen binding domain comprises a VH comprising the amino acid sequence of SEQ ID NO: 6372 (or an amino acid sequence having at least about 80%, 85%, 90%, 95%, or 99% sequence identity thereto) and a VL comprising the amino acid sequence of SEQ ID NO: 240 (or an amino acid sequence having at least about 80%, 85%, 90%, 95%, or 99% sequence identity thereto). In some embodiments, the calreticulin-targeting antigen binding domain comprises a VH comprising the amino acid sequence of SEQ ID NO: 6372 and a VL comprising the amino acid sequence of SEQ ID NO: 240. In some embodiments, the calreticulin-targeting antigen binding domain comprises a VH comprising the amino acid sequence of SEQ ID NO: 234 (or an amino acid sequence having at least about 80%, 85%, 90%, 95%, or 99% sequence identity thereto) and a VL comprising the amino acid sequence of SEQ ID NO: 240 (or an amino acid sequence having at least about 80%, 85%, 90%, 95%, or 99% sequence identity thereto). In some embodiments, the calreticulin-targeting antigen binding domain comprises a VH comprising the amino acid sequence of SEQ ID NO: 234 and a VL comprising the amino acid sequence of SEQ ID NO: 240. In some embodiments, the calreticulin-targeting antigen binding domain comprises a VH comprising the amino acid sequence of SEQ ID NO: 235 (or an amino acid sequence having at least about 80%, 85%, 90%, 95%, or 99% sequence identity thereto) and a VL comprising the amino acid sequence of SEQ ID NO: 240 (or an amino acid sequence having at least about 80%, 85%, 90%, 95%, or 99% sequence identity thereto). In some embodiments, the calreticulin-targeting antigen binding domain comprises a VH comprising the amino acid sequence of SEQ ID NO: 235 and a VL comprising the amino acid sequence of SEQ ID NO: 240. In some embodiments, the calreticulin-targeting antigen binding domain comprises a VH comprising the amino acid sequence of SEQ ID NO: 236 (or an amino acid sequence having at least about 80%, 85%, 90%, 95%, or 99% sequence identity thereto) and a VL comprising the amino acid sequence of SEQ ID NO: 240 (or an amino acid sequence having at least about 80%, 85%, 90%, 95%, or 99% sequence identity thereto). In some embodiments, the calreticulin-targeting antigen binding domain comprises a VH comprising the amino acid sequence of SEQ ID NO: 236 and a VL comprising the amino acid sequence of SEQ ID NO: 240. In some embodiments, the calreticulin-targeting antigen binding domain comprises a VH comprising the amino acid sequence of SEQ ID NO: 237 (or an amino acid sequence having at least about 80%, 85%, 90%, 95%, or 99% sequence identity thereto) and a VL comprising the amino acid sequence of SEQ ID NO: 240 (or an amino acid sequence having at least about 80%, 85%, 90%, 95%, or 99% sequence identity thereto). In some embodiments, the calreticulin-targeting antigen binding domain comprises a VH comprising the amino acid sequence of SEQ ID NO: 237 and a VL comprising the amino acid sequence of SEQ ID NO: 240. In some embodiments, the calreticulin-targeting antigen binding domain comprises a VH comprising the amino acid sequence of SEQ ID NO: 6372 (or an amino acid sequence having at least about 80%, 85%, 90%, 95%, or 99% sequence identity thereto) and a VL comprising the amino acid sequence of SEQ ID NO: 241 (or an amino acid sequence having at least about 80%, 85%, 90%, 95%, or 99% sequence identity thereto). In some embodiments, the calreticulin-targeting antigen binding domain comprises a VH comprising the amino acid sequence of SEQ ID NO: 6372 and a VL comprising the amino acid sequence of SEQ ID NO: 241. In some embodiments, the calreticulin-targeting antigen binding domain comprises a VH comprising the amino acid sequence of SEQ ID NO: 234 (or an amino acid sequence having at least about 80%, 85%, 90%, 95%, or 99% sequence identity thereto) and a VL comprising the amino acid sequence of SEQ ID NO: 241 (or an amino acid sequence having at least about 80%, 85%, 90%, 95%, or 99% sequence identity thereto). In some embodiments, the calreticulin-targeting antigen binding domain comprises a VH comprising the amino acid sequence of SEQ ID NO: 234 and a VL comprising the amino acid sequence of SEQ ID NO: 241. In some embodiments, the calreticulin-targeting antigen binding domain comprises a VH comprising the amino acid sequence of SEQ ID NO: 235 (or an amino acid sequence having at least about 80%, 85%, 90%, 95%, or 99% sequence identity thereto) and a VL comprising the amino acid sequence of SEQ ID NO: 241 (or an amino acid sequence having at least about 80%, 85%, 90%, 95%, or 99% sequence identity thereto). In some embodiments, the calreticulin-targeting antigen binding domain comprises a VH comprising the amino acid sequence of SEQ ID NO: 235 and a VL comprising the amino acid sequence of SEQ ID NO: 241. In some embodiments, the calreticulin-targeting antigen binding domain comprises a VH comprising the amino acid sequence of SEQ ID NO: 236 (or an amino acid sequence having at least about 80%, 85%, 90%, 95%, or 99% sequence identity thereto) and a VL comprising the amino acid sequence of SEQ ID NO: 241 (or an amino acid sequence having at least about 80%, 85%, 90%, 95%, or 99% sequence identity thereto). In some embodiments, the calreticulin-targeting antigen binding domain comprises a VH comprising the amino acid sequence of SEQ ID NO: 236 and a VL comprising the amino acid sequence of SEQ ID NO: 241. In some embodiments, the calreticulin-targeting antigen binding domain comprises a VH comprising the amino acid sequence of SEQ ID NO: 237 (or an amino acid sequence having at least about 80%, 85%, 90%, 95%, or 99% sequence identity thereto) and a VL comprising the amino acid sequence of SEQ ID NO: 241 (or an amino acid sequence having at least about 80%, 85%, 90%, 95%, or 99% sequence identity thereto). In some embodiments, the calreticulin-targeting antigen binding domain comprises a VH comprising the amino acid sequence of SEQ ID NO: 237 and a VL comprising the amino acid sequence of SEQ ID NO: 241. In some embodiments, the calreticulin-targeting antigen binding domain comprises a VH comprising the amino acid sequence of SEQ ID NO: 6372 (or an amino acid sequence having at least about 80%, 85%, 90%, 95%, or 99% sequence identity thereto) and a VL comprising the amino acid sequence of SEQ ID NO: 242 (or an amino acid sequence having at least about 80%, 85%, 90%, 95%, or 99% sequence identity thereto). In some embodiments, the calreticulin-targeting antigen binding domain comprises a VH comprising the amino acid sequence of SEQ ID NO: 6372 and a VL comprising the amino acid sequence of SEQ ID NO: 242. In some embodiments, the calreticulin-targeting antigen binding domain comprises a VH comprising the amino acid sequence of SEQ ID NO: 234 (or an amino acid sequence having at least about 80%, 85%, 90%, 95%, or 99% sequence identity thereto) and a VL comprising the amino acid sequence of SEQ ID NO: 242 (or an amino acid sequence having at least about 80%, 85%, 90%, 95%, or 99% sequence identity thereto). In some embodiments, the calreticulin-targeting antigen binding domain comprises a VH comprising the amino acid sequence of SEQ ID NO: 234 and a VL comprising the amino acid sequence of SEQ ID NO: 242. In some embodiments, the calreticulin-targeting antigen binding domain comprises a VH comprising the amino acid sequence of SEQ ID NO: 235 (or an amino acid sequence having at least about 80%, 85%, 90%, 95%, or 99% sequence identity thereto) and a VL comprising the amino acid sequence of SEQ ID NO: 242 (or an amino acid sequence having at least about 80%, 85%, 90%, 95%, or 99% sequence identity thereto). In some embodiments, the calreticulin-targeting antigen binding domain comprises a VH comprising the amino acid sequence of SEQ ID NO: 235 and a VL comprising the amino acid sequence of SEQ ID NO: 242. In some embodiments, the calreticulin-targeting antigen binding domain comprises a VH comprising the amino acid sequence of SEQ ID NO: 236 (or an amino acid sequence having at least about 80%, 85%, 90%, 95%, or 99% sequence identity thereto) and a VL comprising the amino acid sequence of SEQ ID NO: 242 (or an amino acid sequence having at least about 80%, 85%, 90%, 95%, or 99% sequence identity thereto). In some embodiments, the calreticulin-targeting antigen binding domain comprises a VH comprising the amino acid sequence of SEQ ID NO: 236 and a VL comprising the amino acid sequence of SEQ ID NO: 242. In some embodiments, the calreticulin-targeting antigen binding domain comprises a VH comprising the amino acid sequence of SEQ ID NO: 237 (or an amino acid sequence having at least about 80%, 85%, 90%, 95%, or 99% sequence identity thereto) and a VL comprising the amino acid sequence of SEQ ID NO: 242 (or an amino acid sequence having at least about 80%, 85%, 90%, 95%, or 99% sequence identity thereto). In some embodiments, the calreticulin-targeting antigen binding domain comprises a VH comprising the amino acid sequence of SEQ ID NO: 237 and a VL comprising the amino acid sequence of SEQ ID NO: 242.

In some embodiments, the calreticulin-targeting antigen binding domain comprises a VH comprising the amino acid sequence of SEQ ID NO: 236 and a VL comprising the amino acid sequence of SEQ ID NO: 238.

TABLE 16
Exemplary variable regions of additional calreticulin-targeting antigen binding domains

| SEQ ID | | |
|---|---|---|
| NO | Description | Sequence |
| SEQ ID | BJ092 | QVQLVQSGAEVKKPGASVKVSCKASGYSFTGYYIHWVRQAPGQG |
| NO: 6372 | (VH) | LEWIGYISAYNGASSYNQKFKGRATFTVDTSTSTAYMELRSLRSDD |
| | | MAVYYCASSMDYWGQGTLVTVSS |
| SEQ ID | BJ093 | QVQLVQSGAEVKKPGASVKVSCKASGYSFTGYYIHWVRQAPGQG |
| NO: 234 | (VH) | LEWIGYISAYNGASSYNQKFKGRATFTVDTSTSTAYMELRSLRSDD |
| | | TAVYYCASSMDYWGQGTLVTVSS |
| SEQ ID | BJ094 | QVQLVQSGAEVKKPGASVKVSCKASGYSFTGYYIHWVRQAPGKG |
| NO: 235 | (VH) | LEWIGYISAYNGASSYNQKFKGRATFTVDTSTSTAYMELSSLRSED |
| | | TAVYYCASSMDYWGQGTLVTVSS |
| SEQ ID | BJ095 | QVQLVQSGAEVKKPGASVKVSCKASGYSFTGYYIHWVRQAPGQG |
| NO: 236 | (VH) | LEWIGYISAYNGASSYNQKFKGRATFTVDTSISTAYMELSRLRSDD |
| | | TAVYYCASSMDYWGQGTLVTVSS |
| SEQ ID | BJ096 | QVQLVQSGAEVKKPGASVKVSCKASGYSFTGYYIHWVRQAPGQG |
| NO: 237 | (VH) | LEWIGYISAYNGASSYNQKFKGRATFTVDTSTSTAYMELSSLRSED |
| | | TAVYYCASSMDYWGQGTLVTVSS |
| SEQ ID | VH | QVQLVQSGAEVKKPGASVKVSCKASGYSFTGYYIHWVRQAPGX₁G |
| NO: 244 | consensus | LEWIGYISAYNGASSYNQKFKGRATFTVDTSX₂STAYMELX₃X₄LRS |
| | | DDX₅AVYYCASSMDYWGQGTLVTVSS, wherein: |
| | | X₁ is Q or K, |
| | | X₂ is I or T, |
| | | X₃ is S or R, |
| | | X₄ is R or S, or |
| | | X₅ is Tor M |
| SEQ ID | BJ097 | DVVMTQSPLSLPVTLGQPASISCKSSQSLLDSDGKTYLNWLQQRPG |
| NO: 238 | (VL) | QSPKRLIYLVSKLDSGVPDRFSGSGSGTDFTLKISRVEAEDVGVYHC |
| | | WQGTHFPYTFGQGTKLEIK |
| SEQ ID | BJ098 | DVVMTQTPLSLSVTPGQPASISCKSSQSLLDSDGKTYLNWLLQKPG |
| NO: 239 | (VL) | QSPKLLIYLVSKLDSGVPDRFSGSGSGTDFTLKISRVEAEDVGVYHC |
| | | WQGTHFPYTFGQGTKLEIK |
| SEQ ID | BJ099 | DVVMTQTPLSLSVTPGQPASISCKSSQSLLDSDGKTYLNWLLQKPG |
| NO: 240 | (VL) | QPPKLLIYLVSKLDSGVPDRFSGSGSGTDFTLKISRVEAEDVGVYHC |
| | | WQGTHEPYTFGQGTKLEIK |
| SEQ ID | BJ100 | DVVMTQTPLSSPVTLGQPASISCKSSQSLLDSDGKTYLNWLQQRPG |
| NO: 241 | (VL) | QPPKLLIYLVSKLDSGVPDRFSGSGAGTDFTLKISRVEAEDVGVYH |
| | | CWQGTHEPYTFGQGTKLEIK |
| SEQ ID | BJ101 | DVVMTQSPLSLPVTPGEPASISCKSSQSLLDSDGKTYLNWLLQKPG |
| NO: 242 | (VL) | QSPKLLIYLVSKLDSGVPDRFSGSGSGTDFTLKISRVEAEDVGVYHC |
| | | WQGTHFPYTFGQGTKLEIK |
| SEQ ID | VL | DVVMTQX₁PLSX₂X₃VTX₄GX₅PASISCKSSQSLLDSDGKTYLNWLX₆ |
| NO: 245 | consensus | QX₇PGQX₈PKX₉LIYLVSKLDSGVPDRFSGSGX1₀GTDFTLKISRVEAE |
| | | DVGVYHCWQGTHEPYTFGQGTKLEIK, wherein: |
| | | X₁ is S or T, |
| | | X₂ is L or S, |
| | | X₃ is P or S, |
| | | X₄ is L or P, |
| | | X₅ is Q or E, |
| | | X₆ is Q or L, |
| | | X₇ is R or K, |
| | | X₈ is S or P, |
| | | X₉ is R or L, or |
| | | X₁₀ is S or A |

TABLE 17
Exemplary heavy chain CDRs of calreticulin-targeting antigen binding domains

| VH (SEQ ID NO) | VHCDR1 (SEQ ID NO) | VHCDR2 (SEQ ID NO) | VHCDR3 (SEQ ID NO) |
|---|---|---|---|
| BJ092 (SEQ ID | YSFTGYYIH (SEQ ID | YISAYNGASSYNQKF | SSMDY (SEQ ID NO: |
| NO: 6372) | NO: 6253) | KG (SEQ ID NO: 243) | 6255) |
| BJ093 (SEQ ID | YSFTGYYIH (SEQ ID | YISAYNGASSYNQKF | SSMDY (SEQ ID NO: |
| NO: 234) | NO: 6253) | KG (SEQ ID NO: 243) | 6255) |
| BJ094 (SEQ ID | YSFTGYYIH (SEQ ID | YISAYNGASSYNQKF | SSMDY (SEQ ID NO: |
| NO: 235) | NO: 6253) | KG (SEQ ID NO: 243) | 6255) |
| BJ095 (SEQ ID | YSFTGYYIH (SEQ ID | YISAYNGASSYNQKF | SSMDY (SEQ ID NO: |
| NO: 236) | NO: 6253) | KG (SEQ ID NO: 243) | 6255) |
| BJ096 (SEQ ID | YSFTGYYIH (SEQ ID | YISAYNGASSYNQKF | SSMDY (SEQ ID NO: |
| NO: 237) | NO: 6253) | KG (SEQ ID NO: 243) | 6255) |

TABLE 18
Exemplary light chain CDRs of calreticulin-targeting antigen binding domains

| VL (SEQ ID NO) | VLCDR1 (SEQ ID NO) | VLCDR2 (SEQ ID NO) | VLCDR3 (SEQ ID NO) |
|---|---|---|---|
| BJ097 (SEQ ID | KSSQSLLDSDGKTYLN | LVSKLDS (SEQ ID | WQGTHFPYT (SEQ |
| NO: 238) | (SEQ ID NO: 6259) | NO: 6260) | ID NO: 6261) |
| BJ098 (SEQ ID | KSSQSLLDSDGKTYLN | LVSKLDS (SEQ ID | WQGTHFPYT (SEQ |
| NO: 239) | (SEQ ID NO: 6259) | NO: 6260) | ID NO: 6261) |
| BJ099 (SEQ ID | KSSQSLLDSDGKTYLN | LVSKLDS (SEQ ID | WQGTHFPYT (SEQ |
| NO: 240) | (SEQ ID NO: 6259) | NO: 6260) | ID NO: 6261) |
| BJ100 (SEQ ID | KSSQSLLDSDGKTYLN | LVSKLDS (SEQ ID | WQGTHFPYT (SEQ |
| NO: 241) | (SEQ ID NO: 6259) | NO: 6260) | ID NO: 6261) |
| BJ101 (SEQ ID | KSSQSLLDSDGKTYLN | LVSKLDS (SEQ ID | WQGTHFPYT (SEQ |
| NO: 242) | (SEQ ID NO: 6259) | NO: 6260) | ID NO: 6261) |

TABLE 19
Exemplary calreticulin-targeting antigen binding domains

| Antibody | | | | |
|---|---|---|---|---|
| code | VH code | VH germline | VL code | VL germline |
| BJM0040 | BJ092 (SEQ | IGHV1- | BJ097 (SEQ | IGKV2- |
| | ID NO: 6372) | 18*03 | ID NO: 238) | 30*01 |
| BJM0041 | BJ093 (SEQ | IGHV1- | BJ097 (SEQ | IGKV2- |
| | ID NO: 234) | 18*01 | ID NO: 238) | 30*01 |
| BJM0042 | BJ094 (SEQ | IGHV1- | BJ097 (SEQ | IGKV2- |
| | ID NO: 235) | 2*02 | ID NO: 238) | 30*01 |
| BJM0043 | BJ095 (SEQ | IGHV1- | BJ097 (SEQ | IGKV2- |
| | ID NO: 236) | 2*02 | ID NO: 238) | 30*01 |
| BJM0044 | BJ096 (SEQ | IGHV1- | BJ097 (SEQ | IGKV2- |
| | ID NO: 237) | 2*02 | ID NO: 238) | 30*01 |
| BJM0045 | BJ092 (SEQ | IGHV1- | BJ098 (SEQ | IGKV2- |
| | ID NO: 6372) | 18*03 | ID NO: 239) | 29*02 |
| BJM0046 | BJ093 (SEQ | IGHV1- | BJ098 (SEQ | IGKV2- |
| | ID NO: 234) | 18*01 | ID NO: 239) | 29*02 |
| BJM0047 | BJ094 (SEQ | IGHV1- | BJ098 (SEQ | IGKV2- |
| | ID NO: 235) | 2*02 | ID NO: 239) | 29*02 |
| BJM0048 | BJ095 (SEQ | IGHV1- | BJ098 (SEQ | IGKV2- |
| | ID NO: 236) | 2*02 | ID NO: 239) | 29*02 |
| BJM0049 | BJ096 (SEQ | IGHV1- | BJ098 (SEQ | IGKV2- |
| | ID NO: 237) | 2*02 | ID NO: 239) | 29*02 |
| BJM0050 | BJ092 (SEQ | IGHV1- | BJ099 (SEQ | IGKV2D- |
| | ID NO: 6372) | 18*03 | ID NO: 240) | 29*01 |
| BJM0051 | BJ093 (SEQ | IGHV1- | BJ099 (SEQ | IGKV2D- |
| | ID NO: 234) | 18*01 | ID NO: 240) | 29*01 |
| BJM0052 | BJ094 (SEQ | IGHV1- | BJ099 (SEQ | IGKV2D- |
| | ID NO: 235) | 2*02 | ID NO: 240) | 29*01 |
| BJM0053 | BJ095 (SEQ | IGHV1- | BJ099 (SEQ | IGKV2D- |
| | ID NO: 236) | 2*02 | ID NO: 240) | 29*01 |
| BJM0054 | BJ096 (SEQ | IGHV1- | BJ099 (SEQ | IGKV2D- |
| | ID NO: 237) | 2*02 | ID NO: 240) | 29*01 |
| BJM0055 | BJ092 (SEQ | IGHV1- | BJ100 (SEQ | IGKV2- |
| | ID NO: 6372) | 18*03 | ID NO: 241) | 24*01 |
| BJM0056 | BJ093 (SEQ | IGHV1- | BJ100 (SEQ | IGKV2- |
| | ID NO: 234) | 18*01 | ID NO: 241) | 24*01 |
| BJM0057 | BJ094 (SEQ | IGHV1- | BJ100 (SEQ | IGKV2- |
| | ID NO: 235) | 2*02 | ID NO: 241) | 24*01 |
| BJM0058 | BJ095 (SEQ | IGHV1- | BJ100 (SEQ | IGKV2- |
| | ID NO: 236) | 2*02 | ID NO: 241) | 24*01 |
| BJM0059 | BJ096 (SEQ | IGHV1- | BJ100 (SEQ | IGKV2- |
| | ID NO: 237) | 2*02 | ID NO: 241) | 24*01 |
| BJM0060 | BJ092 (SEQ | IGHV1- | BJ101 (SEQ | IGKV2- |
| | ID NO: 6372) | 18*03 | ID NO: 242) | 28*01 |
| BJM0061 | BJ093 (SEQ | IGHV1- | BJ101 (SEQ | IGKV2- |
| | ID NO: 234) | 18*01 | ID NO: 242) | 28*01 |
| BJM0062 | BJ094 (SEQ | IGHV1- | BJ101 (SEQ | IGKV2- |
| | ID NO: 235) | 2*02 | ID NO: 242) | 28*01 |
| BJM0063 | BJ095 (SEQ | IGHV1- | BJ101 (SEQ | IGKV2- |
| | ID NO: 236) | 2*02 | ID NO: 242) | 28*01 |
| BJM0064 | BJ096 (SEQ | IGHV1- | BJ101 (SEQ | IGKV2- |
| | ID NO: 237) | 2*02 | ID NO: 242) | 28*01 |

### Immune Cell Engagers

The immune cell engagers of the multispecific or multifunctional molecules disclosed herein can mediate binding to, and/or activation of, an immune cell, e.g., an immune effector cell. In some embodiments, the immune cell is chosen from a T cell, an NK cell, a B cell, a dendritic cell, or a macrophage cell engager, or a combination thereof. In some embodiments, the immune cell engager is chosen from one, two, three, or all of a T cell engager, NK cell engager, a B cell engager, a dendritic cell engager, or a macrophage cell engager, or a combination thereof. The immune cell engager can be an agonist of the immune system. In some embodiments, the immune cell engager can be an antibody molecule, a ligand molecule (e.g., a ligand that further comprises an immunoglobulin constant region, e.g., an Fc region), a small molecule, or a nucleotide molecule.

### T Cell Engagers

The present disclosure provides, inter alia, multispecific (e.g., bi-, tri-, quad-specific) or multifunctional molecules, that are engineered to contain one or more T cell engagers that mediate binding to and/or activation of a T cell. Accordingly, in some embodiments, the T cell engager is selected from an antigen binding domain or ligand that binds to (e.g., and in some embodiments activates) one or more of the variable chain of the beta subunit of a TCR (e.g., TCRβV), CD3, TCRα, TCRβ, TCRγ, TCRξ, ICOS, CD28, CD27, HVEM, LIGHT, CD40, 4-1BB, OX40, DR3, GITR, CD30, TIM1, SLAM, CD2, or CD226. In other embodiments, the T cell engager is selected from an antigen binding domain or ligand that binds to and does not activate one or more of TCRβV, CD3, TCRα, TCRβ, TCRγ, TCRξ, ICOS, CD28, CD27, HVEM, LIGHT, CD40, 4-1BB, OX40, DR3, GITR, CD30, TIM1, SLAM, CD2, or CD226. In some embodiments, the T cell engager binds to TCRβV.

In some embodiments, the T cell engager binds to CD3 (e.g., comprises an antigen binding domain that binds to CD3). In some embodiments, the multispecific or multifunctional molecule comprises a T cell engager that binds to CD3 (e.g., comprises an antigen binding domain that binds to CD3) (e.g., comprises an antigen binding domain that binds to CD3) and a calreticulin-targeting antigen binding domain, e.g., as described herein.

In some embodiments, a multispecific or multifunctional molecule (e.g., as described herein) comprises an antigen binding domain that binds to CD3. In some embodiments, the multispecific or multifunctional molecule comprises an antigen binding domain that binds to CD3 and a calreticulin-targeting antigen binding domain, e.g., as described herein.

In some embodiments, the antigen binding domain that binds to CD3 comprises one or more CDRs (e.g., VHCDR1, VHCDR2, VHCDR3, VLCDR1, VLCDR2, and/or VLCDR3) disclosed in Table 26 and/or Table 42, or a sequence having at least 85%, 90%, 95%, or 99% identity thereto. In some embodiments, the antigen binding domain that binds to CD3 comprises one or more framework regions (e.g., VHFWR1, VHFWR2, VHFWR3, VHFWR4, VLFWR1, VLFWR2, VLFWR3, and/or VLFWR4) disclosed in Table 26 and/or Table 42, or a sequence having at least 85%, 90%, 95%, or 99% identity thereto. In some embodiments, the antigen binding domain that binds to CD3 comprises a VH and/or a VL disclosed in Table 27, or a sequence having at least 85%, 90%, 95%, or 99% identity thereto.

TABLE 26
Exemplary heavy chain CDRs and FWRs of CD3-targeting antigen binding domains

| Ab ID | VHFWR1 | VHCDR1 | VHFWR2 | VHCDR2 | VHFWR3 | VHCDR3 | VHFWR4 |
|---|---|---|---|---|---|---|---|
| BJM1210 | QVQLQQ | FTTYW | WVKQR | NFNPNN | KATLTVD | DDYGR | WGQG |
| (murine) | PGTELVK | MH | PGHGLE | GDTNY | KSSSTAY | YYFDY | TTLTV |
| | PGASVKL | (SEQ | WIG | NEKFKT | MQLSSLT | (SEQ ID | SS (SEQ |
| | SCKASGY | ID NO: | (SEQ ID | (SEQ ID | SEDSAVY | NO: | ID NO: |
| | T (SEQ ID | 7456) | NO: | NO: | YCAR | 7460) | 7461) |
| | NO: 7455) | | 7457) | 7458) | (SEQ ID | | |
| | | | | | NO: 7459) | | |
| BKM0020 | QVQLVQ | FTTYW | WVRQA | NFNPNN | RVTMTV | DDYGR | WGQG |
| (humanized) | SGAEVK | MH | PGQGLE | GDTNY | DKSTSTA | YYFDY | TLVTV |
| | KPGASVK | (SEQ | WMG | NEKFKT | YMELRSL | (SEQ ID | SS (SEQ |
| | VSCKASG | ID NO: | (SEQ ID | (SEQ ID | RSDDMA | NO: | ID NO: |
| | YT (SEQ | 7456) | NO: | NO: | VYYCAR | 7460) | 228) |
| | ID NO: | | 6027) | 7458) | (SEQ ID | | |
| | 7467) | | | | NO: 7468) | | |
| BKM0025 | QVQLVQ | FTTYW | WVRQA | NFNPNN | RVTMTV | DDYGR | WGQG |
| (humanized) | SGAEVK | MH | PGQGLE | GDINY | DKSTSTA | YYFDY | TLVTV |
| | KPGASVK | (SEQ | WMG | NEKFKT | YMELRSL | (SEQ ID | SS (SEQ |
| | VSCKASG | ID NO: | (SEQ ID | (SEQ ID | RSDDMA | NO: | ID NO: |
| | YT (SEQ | 7456) | NO: | NO: | VYYCAR | 7460) | 228) |
| | ID NO: | | 6027) | 7458) | (SEQ ID | | |
| | 7467) | | | | NO: 7468) | | |
| BKM0028 | QVQLVQ | FTTYW | WVRQA | NFNPNN | RVTMTV | DDYGR | WGQG |
| (humanized) | SGAEVK | MH | PGKGLE | GDTNY | DKSTSTA | YYFDY | TLVTV |
| | KPGASVK | (SEQ | WMG | NEKFKT | YMELSSL | (SEQ ID | SS (SEQ |
| | VSCKASG | ID NO: | (SEQ ID | (SEQ ID | RSEDTAV | NO: | ID NO: |
| | YT (SEQ | 7456) | NO: | NO: | YYCAR | 7460) | 228) |
| | ID NO: | | 7473) | 7458) | (SEQ ID | | |
| | 7467) | | | | NO: 7474) | | |
| BKM0038 | QVQLVQ | FTTYW | WVRQA | NFNPNN | RVTMTV | DDYGR | WGQG |
| (humanized) | SGAEVK | MH | PGKGLE | GDTNY | DKSTSTA | YYFDY | TLVTV |
| | KPGASVK | (SEQ | WMG | NEKFKT | YMELSSL | (SEQ ID | SS (SEQ |
| | VSCKASG | ID NO: | (SEQ ID | (SEQ ID | RSEDTAV | NO: | ID NO: |
| | YT (SEQ | 7456) | NO: | NO: | YYCAR | 7460) | 228) |
| | ID NO: | | 7473) | 7458) | (SEQ ID | | |
| | 7467) | | | | NO: 7474) | | |

TABLE 42
Exemplary light chain CDRs and FWRs of CD3-targeting antigen binding domains

| Ab ID | VLFWR1 | VLCDR1 | VLFWR2 | VLCDR2 | VLFWR3 | VLCDR3 | VLFWR4 |
|---|---|---|---|---|---|---|---|
| BJM1210 | DIVMSQS | KSSQSL | WYQQ | WAFTR | GVPDRFT | KQSFIL | FGGGT |
| (murine) | PSSLAVS | LNSRTR | KPGQA | ES (SEQ | GSGSGTD | RT (SEQ | KLEIK |
| | AGEKVT | KNYLA | PKLLIY | ID NO: | FTLTISSV | ID NO: | (SEQ ID |
| | MSC (SEQ | (SEQ ID | (SEQ ID | 7464) | QAEDLAI | 7466) | NO: |
| | ID NO: | NO: | NO: | | YYC (SEQ | | 6284) |
| | 7462) | 7463) | 7433) | | ID NO: | | |
| | | | | | 7465) | | |
| BKM0020 | DIOMTQS | KSSQSL | WYQQ | WAFTR | GVPSRFSG | KQSFIL | FGGGT |
| (humanized) | PSTLSAS | LNSRTR | KPGKA | ES (SEQ | SGSGTEFT | RT (SEQ | KVEIK |
| | VGDRVTI | KNYLA | PKLLIY | ID NO: | LTISSLQP | ID NO: | (SEQ ID |
| | TC (SEQ | (SEQ ID | (SEQ ID | 7464) | DDFATYY | 7466) | NO: |
| | ID NO: | NO: | NO: | | C (SEQ ID | | 233) |
| | 6093) | 7463) | 280) | | NO: 7469) | | |
| BKM0025 | EIVMTQS | KSSQSL | WYQQ | WAFTR | GIPDRFSG | KQSFIL | FGGGT |
| (humanized) | PATLSLS | LNSRTR | KPGLA | ES (SEQ | SGSGTDFT | RT (SEQ | KVEIK |
| | PGERATL | KNYLA | PRLLIY | ID NO: | LTISRLEP | ID NO: | (SEQ ID |
| | SC (SEQ | (SEQ ID | (SEQ ID | 7464) | EDFAVYY | 7466) | NO: |
| | ID NO: | NO: | NO: | | C (SEQ ID | | 233) |
| | 7470) | 7463) | 7471) | | NO: 7472) | | |
| BKM0028 | EIVMTQS | KSSQSL | WYQQ | WAFTR | GIPDRESG | KQSFIL | FGGGT |
| (humanized) | PATLSLS | LNSRTR | KPGLA | ES (SEQ | SGSGTDFT | RT (SEQ | KVEIK |
| | PGERATL | KNYLA | PRLLIY | ID NO: | LTISRLEP | ID NO: | (SEQ ID |
| | SC (SEQ | (SEQ ID | (SEQ ID | 7464) | EDFAVYY | 7466) | NO: |
| | ID NO: | NO: | NO: | | C (SEQ ID | | 233) |
| | 7470) | 7463) | 7471) | | NO: 7472) | | |
| BKM0038 | DIQMTQS | KSSQSL | WYQQ | WAFTR | GVPSRFSG | KQSFIL | FGGGT |
| (humanized) | PSSLSAS | LNSRTR | KPGKA | ES (SEQ | SGSGTDFT | RT (SEQ | KVEIK |
| | VGDRVTI | KNYLA | PKLLIY | ID NO: | LTISSLQP | ID NO: | (SEQ ID |
| | TC (SEQ | (SEQ ID | (SEQ ID | 7464) | EDFATYY | 7466) | NO: |
| | ID NO: | NO: | NO: | | C (SEQ ID | | 233) |
| | 7475) | 7463) | 280) | | NO: 7476) | | |

TABLE 27
Exemplary variable regions of CD3-targeting antigen binding domains

| SEQ | | |
|---|---|---|
| ID NO | Ab ID | Sequence |
| 7477 | BJM1210 | QVQLQQPGTELVKPGASVKLSCKASGYTFTTYWMHWVKQRPGH |
| | (murine) VH | GLEWIGNFNPNNGDTNYNEKFKTKATLTVDKSSSTAYMQLSSLTS |
| | | EDSAVYYCARDDYGRYYFDYWGQGTTLTVSS |
| 7478 | BKM0020 | QVQLVQSGAEVKKPGASVKVSCKASGYTFTTYWMHWVRQAPGQ |
| | (humanized) | GLEWMGNFNPNNGDTNYNEKFKTRVTMTVDKSTSTAYMELRSL |
| | VH | RSDDMAVYYCARDDYGRYYFDYWGQGTLVTVSS |
| 7478 | BKM0025 | QVQLVQSGAEVKKPGASVKVSCKASGYTFTTYWMHWVRQAPGQ |
| | (humanized) | GLEWMGNFNPNNGDTNYNEKFKTRVTMTVDKSTSTAYMELRSL |
| | VH | RSDDMAVYYCARDDYGRYYFDYWGQGTLVTVSS |
| 7479 | BKM0028 | QVQLVQSGAEVKKPGASVKVSCKASGYTFTTYWMHWVRQAPGK |
| | (humanized) | GLEWMGNFNPNNGDTNYNEKFKTRVTMTVDKSTSTAYMELSSLR |
| | VH | SEDTAVYYCARDDYGRYYFDYWGQGTLVTVSS |
| 7479 | BKM0038 | QVQLVQSGAEVKKPGASVKVSCKASGYTFTTYWMHWVRQAPGK |
| | (humanized) | GLEWMGNFNPNNGDTNYNEKFKTRVTMTVDKSTSTAYMELSSLR |
| | VH | SEDTAVYYCARDDYGRYYFDYWGQGTLVTVSS |
| 7480 | BJM1210 | DIVMSQSPSSLAVSAGEKVTMSCKSSQSLLNSRTRKNYLAWYQQK |
| | (murine) VL | PGQAPKLLIYWAFTRESGVPDRFTGSGSGTDFTLTISSVQAEDLAIY |
| | | YCKQSFILRTFGGGTKLEIK |
| 7481 | BKM0020 | DIQMTQSPSTLSASVGDRVTITCKSSQSLLNSRTRKNYLAWYQQK |
| | (humanized) | PGKAPKLLIYWAFTRESGVPSRFSGSGSGTEFTLTISSLQPDDFATY |
| | VL | YCKQSFILRTFGGGTKVEIK |
| 7482 | BKM0025 | EIVMTQSPATLSLSPGERATLSCKSSQSLLNSRTRKNYLAWYQQKP |
| | (humanized) | GLAPRLLIYWAFTRESGIPDRFSGSGSGTDFTLTISRLEPEDFAVYY |
| | VL | CKQSFILRTFGGGTKVEIK |
| 7482 | BKM0028 | EIVMTQSPATLSLSPGERATLSCKSSQSLLNSRTRKNYLAWYQQKP |
| | (humanized) | GLAPRLLIYWAFTRESGIPDRFSGSGSGTDFTLTISRLEPEDFAVYY |
| | VL | CKQSFILRTFGGGTKVEIK |
| 7483 | BKM0038 | DIQMTQSPSSLSASVGDRVTITCKSSQSLLNSRTRKNYLAWYQQKP |
| | (humanized) | GKAPKLLIYWAFTRESGVPSRFSGSGSGTDFTLTISSLQPEDFATYY |
| | VL | CKQSFILRTFGGGTKVEIK |

In some embodiments, the multifunctional molecule (e.g., an scFv, e.g., a bispecific scFv) comprises the amino acid sequence (or a CDR, VH, or VL sequence comprised therein) below, or an amino acid sequence having at least 85%, 90%, 95%, or 99% identity thereto:

(SEQ ID NO: 7452)
EVQLVESGGGLVQPGKSLKLSCEASGFTFSGYGMHWVRQAPGRGLESVA
YITSSSINIKYADAVKGRFTVSRDNAKNLLFLQMNILKSEDTAMYYCAR
FDWDKNYWGQGTMVTVSSGGGGSGGGGSGGGGSGGGGSDIQMTQSPSSL
PASLGDRVTINCQASQDISNYLNWYQQKPGKAPKLLIYYTNKLADGVPS
RFSGSGSGRDSSFTISSLESEDIGSYYCQQYYNYPWTFGPGTKLEIKGG
GGSTIKPCPPCKCPAPNAAGGPSVFIFPPKIKDVLMISLSPIVTCVVVD
VSEDDPDVQISWFVNNVEVHTAQTQTHREDYNSTLRVVSALPIQHQDWM
SGKEFKCKVNNKDLGAPIERTISKPKGSVRAPQVCVLPPPEEEMTKKQV
TLSCAVTDFMPEDIYVEWTNNGKTELNYKNTEPVLDSDGSYFMVSKLRV
EKKNWVERNSYSCSVVHEGLHNHHTTKSFSRTPGK.

In some embodiments, the multifunctional molecule (e.g., a bispecific antibody molecule) comprises the amino acid sequence (or a CDR (underlined), VH, or constant region sequence comprised therein), below, or an amino acid sequence having at least 85%, 90%, 95%, or 99% identity thereto:

(SEQ ID NO: 7484)
EVQLVESGGGLVQPGGSLRLSCAASGFTFSEYWMNWLRQAPGKGLEWVG
VIKYKYSNYATEFAESVKGRFTISRDDSKSSVYLQMNSLKTEDTAVYYC
ARGRDVQDYWGQGTMVTVSSASTKGPSVFPLAPSSKSTSGGTAALGCLV
KDYFPEPVTVSWNSGALTSGVHTFPAVLQSSGLYSLSSVVTVPSSSLGT
QTYICNVNHKPSNTKVDKRVEPKSCDKTHTCPPCPAPELLGGPSVFLFP
PKPKDTLMISRTPEVTCVVVDVSHEDPEVKFNWYVDGVEVHNAKTKPRE
EQYASTYRVVSVLTVLHQDWLNGKEYKCKVSNKALPAPIEKTISKAKGQ
PREPQVYTLPPCREEMTKNQVSLWCLVKGFYPSDIAVEWESNGQPENNY
KTTPPVLDSDGSFFLYSKLTVDKSRWQQGNVFSCSVMHEALHNHYTQKS
LSLSPGK

In some embodiments, the multifunctional molecule (e.g., a bispecific antibody molecule) comprises the amino acid sequence (or a CDR (underlined), VL, or constant region sequence comprised therein), below, or an amino acid sequence having at least 85%, 90%, 95%, or 99% identity thereto:

(SEQ ID NO: 7485)
DIQLTQSPSFLSASVGDRVTITCSTSSSVTTNYLHWYQQKPGKAPKLLI
YSTSNLASGVPSRFSGSGSGTEYTLTISSLQPEDFATYYCQQCLSSPCT
FGQGTKLEIKRTVAAPSVFIFPPSDEQLKSGTASVVCLLNNFYPREAKV
QWKVDNALQSGNSQESVTEQDSKDSTYSLSSTLTLSKADYEKHKVYACE
VTHQGLSSPVTKSFNRGEC

In some embodiments, the multifunctional molecule (e.g., an scFv, e.g., a bispecific scFv) comprises the amino acid sequence (or a CDR (underlined), VH, or VL sequence comprised therein), below, or an amino acid sequence having at least 85%, 90%, 95%, or 99% identity thereto:

(SEQ ID NO: 7486)
QVQLVQSGAEVKKPGASVKVSCKASGYTFTTYWMHWVRQAPGQGLEWMG
NFNPNNGDTNYNEKFKTRVTMTVDKSTSTAYMELRSLRSDDMAVYYCAR
DDYGRYYFDYWGQGTLVTVSSGGGGSGGGGSGGGGSGGGGSDIQMTQSP
STLSASVGDRVTITCKSSQSLLNSRTRKNYLAWYQQKPGKAPKLLIYWA
FTRESGVPSRFSGSGSGTEFTLTISSLQPDDFATYYCKQSFILRTFGGG
TKVEIKDKTHTCPPCPAPELLGGPSVFLFPPKPKDTLMISRTPEVTCVV
VDVSHEDPEVKFNWYVDGVEVHNAKTKPREEQYASTYRVVSVLTVLHQD
WLNGKEYKCKVSNKALPAPIEKTISKAKGQPREPQVCTLPPSREEMTKN
QVSLSCAVKGFYPSDIAVEWESNGQPENNYKTTPPVLDSDGSFFLVSKL
TVDKSRWQQGNVFSCSVMHEALHNHYTQKSLSLSPGK

### Human T Cell Receptor (TCR) Complex

T cell receptors (TCR) can be found on the surface of T cells. TCRs recognize antigens, e.g., peptides, presented on, e.g., bound to, major histocompatibility complex (MHC) molecules on the surface of cells, e.g., antigen-presenting cells. TCRs are heterodimeric molecules and can comprise an alpha chain, a beta chain, a gamma chain or a delta chain. TCRs comprising an alpha chain and a beta chain are also referred to as TCRαβ. The TCR beta chain consists of the following regions (also known as segments): variable (V), diversity (D), joining (J) and constant (C) (see Mayer G. and Nyland J. (2010) Chapter 10: Major Histocompatibility Complex and T-cell Receptors-Role in Immune Responses. In: Microbiology and Immunology on-line, University of South Carolina School of Medicine). The TCR alpha chain consists of V, J and C regions. The rearrangement of the T-cell receptor (TCR) through somatic recombination of V (variable), D (diversity), J (joining), and C (constant) regions is a defining event in the development and maturation of a T cell. TCR gene rearrangement takes place in the thymus.

TCRs can comprise a receptor complex, known as the TCR complex, which comprises a TCR heterodimer comprising of an alpha chain and a beta chain, and dimeric signaling molecules, e.g., CD3 co-receptors, e.g., CD3δ/ε, and/or CD3γ/ε.

### TCR Beta V (TCRBV)

Diversity in the immune system enables protection against a huge array of pathogens. Since the germline genome is limited in size, diversity is achieved not only by the process of V(D)J recombination but also by junctional (junctions between V-D and D-J segments) deletion of nucleotides and addition of pseudo-random, non-templated nucleotides. The TCR beta gene undergoes gene arrangement to generate diversity.

The TCR V beta repertoire varies between individuals and populations because of, e.g., 7 frequently occurring inactivating polymorphisms in functional gene segments and a large insertion/deletion-related polymorphism encompassing 2 V beta gene segments.

This disclosure provides, inter alia, antibody molecules and fragments thereof, that bind, e.g., specifically bind, to a human TCR beta V chain (TCRβV), e.g., a TCRβV gene family (also referred to as a group), e.g., a TCRβV subfamily (also referred to as a subgroup), e.g., as described herein. TCR beta V families and subfamilies are known in the art, e.g., as described in Yassai et al., (2009) Immunogenetics 61(7)pp:493-502; Wei S. and Concannon P. (1994) Human Immunology 41(3) pp: 201-206. The antibodies described herein can be recombinant antibodies, e.g., recombinant non-murine antibodies, e.g., recombinant human or humanized antibodies.

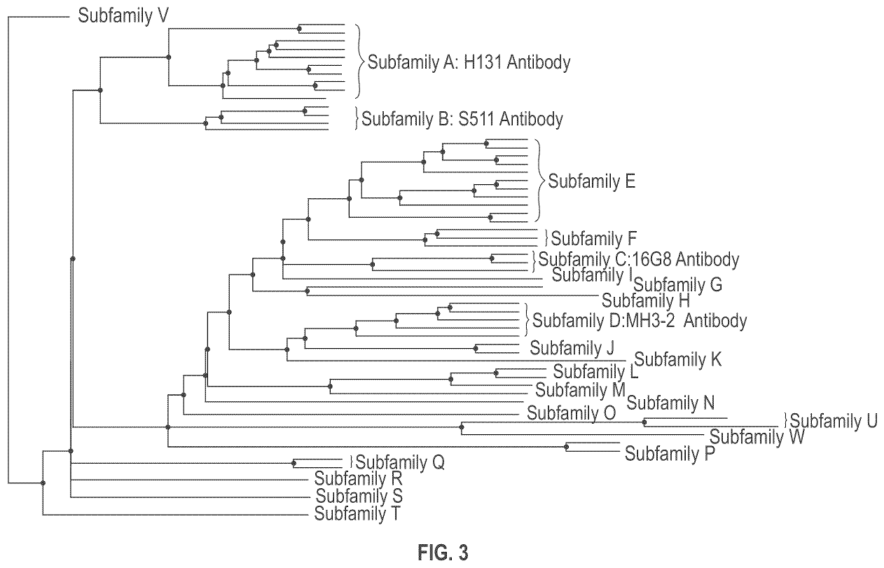

In an aspect, the disclosure provides an anti-TCRβV antibody molecule that binds to human TCRβV, e.g., a TCRβV family, e.g., gene family or a variant thereof. In some embodiments a TCRBV gene family comprises one or more subfamilies, e.g., as described herein, e.g., in FIG. 3, Table 28 or Table 29. In some embodiments, the TCRβV gene family comprises: a TCRβ V6 subfamily, a TCRβ V10 subfamily, a TCRβ V12 subfamily, a TCRβ V5 subfamily, a TCRβ V7 subfamily, a TCRβ V11 subfamily, a TCRβ V14 subfamily, a TCRβ V16 subfamily, a TCRβ V18 subfamily, a TCRβ V9 subfamily, a TCRβ V13 subfamily, a TCRβ V4 subfamily, a TCRβ V3 subfamily, a TCRβ V2 subfamily, a TCRβ V15 subfamily, a TCRβ V30 subfamily, a TCRβ V19 subfamily, a TCRβ V27 subfamily, a TCRβ V28 subfamily, a TCRβ V24 subfamily, a TCRβ V20 subfamily, TCRβ V25 subfamily, a TCRβ V29 subfamily, a TCRβ V1 subfamily, a TCRβ V17 subfamily, a TCRβ V21 subfamily, a TCRβ V23 subfamily, or a TCRβ V26 subfamily.

In some embodiments, TCRβ V6 subfamily is also known as TCRβ V13.1. In some embodiments, the TCRβ V6 subfamily comprises: TCRβ V6-4*01, TCRβ V6-4*02, TCRβ V6-9*01, TCRβ V6-8*01, TCRβ V6-5*01, TCRβ V6-6*02, TCRβ V6-6*01, TCRβ V6-2*01, TCRβ V6-3*01 or TCRβ V6-1*01, or a variant thereof. In some embodiments, TCRβ V6 comprises TCRβ V6-4*01, or a variant thereof. In some embodiments, TCRβ V6 comprises TCRβ V6-4*02, or a variant thereof. In some embodiments, TCRβ V6 comprises TCRβ V6-9*01, or a variant thereof. In some embodiments, TCRβ V6 comprises TCRβ V6-8*01, or a variant thereof. In some embodiments, TCRβ V6 comprises TCRβ V6-5*01, or a variant thereof. In some embodiments, TCRβ V6 comprises TCRβ V6-6*02, or a variant thereof. In some embodiments, TCRβ V6 comprises TCRβ V6-6*01, or a variant thereof. In some embodiments, TCRβ V6 comprises TCRβ V6-2*01, or a variant thereof. In some embodiments, TCRβ V6 comprises TCRβ V6-3*01, or a variant thereof. In some embodiments, TCRβ V6 comprises TCRβ V6-1*01, or a variant thereof.

In some embodiments, TCRβ V6 comprises TCRβ V6-5*01, or a variant thereof. In some embodiments, TCRβ V6, e.g., TCRβ V6-5*01, is recognized, e.g., bound, by SEQ ID NO: 1 and/or SEQ ID NO: 2. In some embodiments, TCRβ V6, e.g., TCRβ V6-5*01, is recognized, e.g., bound, by SEQ ID NO: 9 and/or SEQ ID NO: 10. In some embodiments, TCRβ V6 is recognized, e.g., bound, by SEQ ID NO: 9 and/or SEQ ID NO: 11.

In some embodiments, TCRβ V10 subfamily is also known as TCRβ V12. In some embodiments, the TCRβ V10 subfamily comprises: TCRβ V10-1*01, TCRβ V10-1*02, TCRβ V10-3*01 or TCRβ V10-2*01, or a variant thereof.

In some embodiments, TCRβ V12 subfamily is also known as TCRβ V8.1. In some embodiments, the TCRβ V12 subfamily comprises: TCRβ V12-4*01, TCRβ V12-3*01, or TCRβ V12-5*01, or a variant thereof. In some embodiments, TCRβ V12 is recognized, e.g., bound, by SEQ ID NO: 15 and/or SEQ ID NO: 16. In some embodiments, TCRβ V12 is recognized, e.g., bound, by any one of SEQ ID NOs 23-25, and/or any one of SEQ ID NO: 26-30:

In some embodiments, the TCRβ V5 subfamily is chosen from: TCRβ V5-5*01, TCRβ V5-6*01, TCRβ V5-4*01, TCRβ V5-8*01, TCRβ V5-1*01, or a variant thereof.

In some embodiments, the TCRβ V7 subfamily comprises TCRβ V7-7*01, TCRβ V7-6*01, TCRβ V7-8*02, TCRβ V7-4*01, TCRβ V7-2*02, TCRβ V7-2*03, TCRβ V7-2*01, TCRβ V7-3*01, TCRβ V7-9*03, or TCRβ V7-9*01, or a variant thereof.

In some embodiments, the TCRβ V11 subfamily comprises: TCRβ V11-1*01, TCRβ V11-2*01 or TCRβ V11-3*01, or a variant thereof.

In some embodiments, the TCRβ V14 subfamily comprises TCRβ V14*01, or a variant thereof.

In some embodiments, the TCRβ V16 subfamily comprises TCRβ V16*01, or a variant thereof.

In some embodiments, the TCRβ V18 subfamily comprises TCRβ V18*01, or a variant thereof.

In some embodiments, the TCRβ V9 subfamily comprises TCRβ V9*01 or TCRβ V9*02, or a variant thereof.

In some embodiments, the TCRβ V13 subfamily comprises TCRβ V13*01, or a variant thereof.

In some embodiments, the TCRβ V4 subfamily comprises TCRβ V4-2*01, TCRβ V4-3*01, or TCRβ V4-1*01, or a variant thereof.

In some embodiments, the TCRβ V3 subfamily comprises TCRβ V3-1*01, or a variant thereof.

In some embodiments, the TCRβ V2 subfamily comprises TCRβ V2*01, or a variant thereof.

In some embodiments, the TCRβ V15 subfamily comprises TCRβ V15*01, or a variant thereof.

In some embodiments, the TCRβ V30 subfamily comprises TCRβ V30*01, or TCRβ V30*02, or a variant thereof.

In some embodiments, the TCRβ V19 subfamily comprises TCRβ V19*01, or TCRβ V19*02, or a variant thereof.

In some embodiments, the TCRβ V27 subfamily comprises TCRβ V27*01, or a variant thereof.

In some embodiments, the TCRβ V28 subfamily comprises TCRβ V28*01, or a variant thereof.

In some embodiments, the TCRβ V24 subfamily comprises TCRβ V24-1*01, or a variant thereof.

In some embodiments, the TCRβ V20 subfamily comprises TCRβ V20-1*01, or TCRβ V20-1*02, or a variant thereof.

In some embodiments, the TCRβ V25 subfamily comprises TCRβ V25-1*01, or a variant thereof.

In some embodiments, the TCRβ V29 subfamily comprises TCRβ V29-1*01, or a variant thereof.

TABLE 28
List of TCRβV subfamilies and subfamily members

| Reference | | |
|---|---|---|
| in FIG. 3 | Subfamily | Subfamily members |
| A | TCRβ V6 | TCRβ V6-4*01, TCRβ V6-4*02, TCRβ V6-9*01, TCRβ V6- |
| | Also referred to as: | 8*01, TCRβ V6-5*01, TCRβ V6-6*02, TCRβ V6-6*01, |
| | TCR Vβ 13.1 | TCRβ V6-2*01, TCRβ V6-3*01 or TCRβ V6-1*01. |
| B | TCRβ V10 | TCRβ V10-1*01, TCRβ V10-1*02, TCRβ V10-3*01 or |
| | Also referred to as: | TCRβ V10-2*01 |
| | TCRB V12 | |
| C | TCRβ V12 | TCRβ V12-4*01, TCRβ V12-3*01, or TCRβ V12-5*01 |
| | Also referred to as: | |
| | TCRβ V8.1 | |
| D | TCRβ V5 | TCRβ V5-5*01, TCRβ V5-6*01, TCRβ V5-4*01, TCRβ V5- |
| | | 8*01, TCRβ V5-1*01 |
| E | TCRβ V7 | TCRβ V7-7*01, TCRβ V7-6*01, TCRβ V7 -8*02, TCRβ V7- |
| | | 4*01, TCRβ V7-2*02, TCRβ V7-2*03, TCRβ V7-2*01, |
| | | TCRβ V7-3*01, TCRβ V7-9*03, or TCRβ V7-9*01 |
| F | TCRβ V11 | TCRβ V11-1*01, TCRβ V11-2*01 or TCRβ V11-3*01 |
| G | TCRβ V14 | TCRβ V14*01 |
| H | TCRβ V16 | TCRβ V16*01 |
| I | TCRβ V18 | TCRβ V18*01 |
| J | TCRβ V9 | TCRβ V9*01 or TCRβ V9*02 |
| K | TCRβ V13 | TCRβ V13*01 |
| L | TCRβ V4 | TCRβ V4-2*01, TCRβ V4-3*01, or TCRβ V4-1*01 |
| M | TCRβ V3 | TCRβ V3-1*01 |
| N | TCRβ V2 | TCRβ V2*01 |
| O | TCRβ V15 | TCRβ V15*01 |
| P | TCRβ V30 | TCRβ V30*01, or TCRβ V30*02 |
| Q | TCRβ V19 | TCRβ V19*01, or TCRβ V19*02 |
| R | TCRβ V27 | TCRβ V27*01. |
| S | TCRβ V28 | TCRβ V28*01. |
| T | TCRβ V24 | TCRβ V24-1*01 |
| U | TCRβ V20 | TCRβ V20-1*01, or TCRβ V20-1*02 |
| V | TCRβ V25 | TCRβ V25-1*01 |
| W | TCRβ V29 | TCRβ V29-1*01 |

TABLE 29
Additional TCRβV subfamilies

Subfamily
TCRβ V1
TCRβ V17
TCRβ V21
TCRβ V23
TCRβ V26

### Anti-TCRβV Antibodies

Disclosed herein, is the discovery of a novel class of antibodies, i.e. anti-TCRβV antibody molecules disclosed herein, which despite having low sequence similarity (e.g., low sequence identity among the different antibody molecules that recognize different TCRβV subfamilies), recognize a structurally conserved region, e.g., domain, on the TCRβV protein and have a similar function (e.g., a similar cytokine profile). Thus, the anti-TCRβV antibody molecules disclosed herein share a structure-function relationship.

In some embodiments, the anti-TCRβV antibody molecules disclosed herein do not recognize, e.g., bind to, an interface of a TCRβV:TCRalpha complex.

In some embodiments, the anti-TCRβV antibody molecules disclosed herein do not recognize, e.g., bind to, a constant region of a TCRβV protein. An exemplary antibody that binds to a constant region of a TCRβV region is JOVI.1 as described in Viney et al., (Hybridoma. 1992 December; 11(6):701-13).

In some embodiments, the anti-TCRβV antibody molecules disclosed herein do not recognize, e.g., bind to, one or more (e.g., all) of a complementarity determining region (e.g., CDR1, CDR2 and/or CDR3) of a TCRβV protein.

In some embodiments, the anti-TCRβV antibody molecules disclosed herein binds (e.g., specifically binds) to a TCRβV region. In some embodiments, binding of anti-TCRβV antibody molecules disclosed herein results in a cytokine profile that differs from a cytokine profile of a T cell engager that binds to a receptor or molecule other than a TCRβV region (“a non-TCRβV-binding T cell engager”). In some embodiments, the non-TCRβV-binding T cell engager comprises an antibody that binds to a CD3 molecule (e.g., CD3 epsilon (CD3e) molecule); or a TCR alpha (TCRα) molecule. In some embodiments, the non-TCRβV-binding T cell engager is an OKT3 antibody or an SP34-2 antibody.

In an aspect, the disclosure provides an anti-TCRβV antibody molecule that binds to human TCRβV, e.g., a TCRβV gene family, e.g., one or more of a TCRβV subfamily, e.g., as described herein, e.g., in FIG. 3, Table 28, or Table 29. In some embodiments, the anti-TCRβV antibody molecule binds to one or more TCRβV subfamilies chosen from: a TCRβ V6 subfamily, a TCRβ V10 subfamily, a TCRβ V12 subfamily, a TCRβ V5 subfamily, a TCRβ V7 subfamily, a TCRβ V11 subfamily, a TCRβ V14 subfamily, a TCRβ V16 subfamily, a TCRβ V18 subfamily, a TCRβ V9 subfamily, a TCR P V13 subfamily, a TCRβ V4 subfamily, a TCRβ V3 subfamily, a TCRβ V2 subfamily, a TCRβ V15 subfamily, a TCRβ V30 subfamily, a TCRβ V19 subfamily, a TCRβ V27 subfamily, a TCRβ V28 subfamily, a TCRβ V24 subfamily, a TCRβ V20 subfamily, TCRβ V25 subfamily, a TCRβ V29 subfamily, a TCRβ V1 subfamily, a TCRβ V17 subfamily, a TCRβ V21 subfamily, a TCRβ V23 subfamily, or a TCRβ V26 subfamily, or a variant thereof.

In some embodiments, the anti-TCRβV antibody molecule binds to a TCRβ V6 subfamily comprising: TCRβ V6-4*01, TCRβ V6-4*02, TCRβ V6-9*01, TCRβ V6-8*01, TCRβ V6-5*01, TCRβ V6-6*02, TCRβ V6-6*01, TCRβ V6-2*01, TCRβ V6-3*01 or TCRβ V6-1*01, or a variant thereof. In some embodiments the TCRβ V6 subfamily comprises TCRβ V6-5*01, or a variant thereof. In some embodiments, TCRβ V6 comprises TCRβ V6-4*01, or a variant thereof. In some embodiments, TCRβ V6 comprises TCRβ V6-4*02, or a variant thereof. In some embodiments, TCRβ V6 comprises TCRβ V6-9*01, or a variant thereof. In some embodiments, TCRβ V6 comprises TCRβ V6-8*01, or a variant thereof. In some embodiments, TCRβ V6 comprises TCRβ V6-5*01, or a variant thereof. In some embodiments, TCRβ V6 comprises TCRβ V6-6*02, or a variant thereof. In some embodiments, TCRβ V6 comprises TCRβ V6-6*01, or a variant thereof. In some embodiments, TCRβ V6 comprises TCRβ V6-2*01, or a variant thereof. In some embodiments, TCRβ V6 comprises TCRβ V6-3*01, or a variant thereof. In some embodiments, TCRβ V6 comprises TCRβ V6-1*01, or a variant thereof.

In some embodiments, the anti-TCRβV antibody molecule binds to a TCRβ V10 subfamily comprising: TCRβ V10-1*01, TCRβ V10-1*02, TCRβ V10-3*01 or TCRβ V10-2*01, or a variant thereof.

In some embodiments, the anti-TCRβV antibody molecule binds to a TCRβ V12 subfamily comprising: TCRβ V12-4*01, TCRβ V12-3*01 or TCRβ V12-5*01, or a variant thereof.

In some embodiments, the anti-TCRβV antibody molecule binds to a TCRβ V5 subfamily comprising: TCRβ V5-5*01, TCRβ V5-6*01, TCRβ V5-4*01, TCRβ V5-8*01, TCRβ V5-1*01, or a variant thereof.

In some embodiments, the anti-TCRβV antibody molecule does not bind to TCRβ V12, or binds to TCRβ V12 with an affinity and/or binding specificity that is less than (e.g., less than about 10%, 20%, 30%, 40%, 50%, 60%, 70%, 80%, 90% or about 2-, 5-, or 10-fold) the affinity and/or binding specificity of the 16G8 murine antibody or a humanized version thereof as described in U.S. Pat. No. 5,861,155.

In some embodiments, the anti-TCRβV antibody molecule binds to TCRβ V12 with an affinity and/or binding specificity that is greater than (e.g., greater than about 10%, 20%, 30%, 40%, 50%, 60%, 70%, 80%, 90% or about 2-, 5-, or 10-fold) the affinity and/or binding specificity of the 16G8 murine antibody or a humanized version thereof as described in U.S. Pat. No. 5,861,155.

In some embodiments, the anti-TCRβV antibody molecule binds to a TCRβV region other than TCRβ V12 (e.g., TCRβV region as described herein, e.g., TCRβ V6 subfamily (e.g., TCRβ V6-5*01) with an affinity and/or binding specificity that is greater than (e.g., greater than about 10%, 20%, 30%, 40%, 50%, 60%, 70%, 80%, 90% or about 2-, 5-, or 10-fold) the affinity and/or binding specificity of the 16G8 murine antibody or a humanized version thereof as described in U.S. Pat. No. 5,861,155.

In some embodiments, the anti-TCRβV antibody molecule does not bind to TCRβ V5-5*01 or TCRβ V5-1*01, or binds to TCRβ V5-5*01 or TCRβ V5-1*01 with an affinity and/or binding specificity that is less than (e.g., less than about 10%, 20%, 30%, 40%, 50%, 60%, 70%, 80%, 90% or about 2-, 5-, or 10-fold) the affinity and/or binding specificity of the TM23 murine antibody or a humanized version thereof as described in U.S. Pat. No. 5,861,155.

In some embodiments, the anti-TCRβV antibody molecule binds to TCRβ V5-5*01 or TCRβ V5-1*01 with an affinity and/or binding specificity that is greater than (e.g., greater than about 10%, 20%, 30%, 40%, 50%, 60%, 70%, 80%, 90% or about 2-, 5-, or 10-fold) the affinity and/or binding specificity of the TM23 murine antibody or a humanized version thereof as described in U.S. Pat. No. 5,861,155.

In some embodiments, the anti-TCRβV antibody molecule binds to a TCRβV region other than TCRβ V5-5*01 or TCRβ V5-1*01 (e.g., TCRβV region as described herein, e.g., TCR P V6 subfamily (e.g., TCRβ V6-5*01) with an affinity and/or binding specificity that is greater than (e.g., greater than about 10%, 20%, 30%, 40%, 50%, 60%, 70%, 80%, 90% or about 2-, 5-, or 10-fold) the affinity and/or binding specificity of the TM23 murine antibody or a humanized version thereof as described in U.S. Pat. No. 5,861,155.

### Anti-TCRβ V6 Antibodies

Accordingly, in one aspect, the disclosure provides an anti-TCRβV antibody molecule that binds to human TCRβ V6, e.g., a TCRβ V6 subfamily comprising: TCRβ V6-4*01, TCRβ V6-4*02, TCRβ V6-9*01, TCRβ V6-8*01, TCRβ V6-5*01, TCRβ V6-6*02, TCRβ V6-6*01, TCRβ V6-2*01, TCRβ V6-3*01 or TCRβ V6-1*01. In some embodiments the TCRβ V6 subfamily comprises TCRβ V6-5*01 or a variant thereof. In some embodiments, TCRβ V6 comprises TCRβ V6-4*01, or a variant thereof. In some embodiments, TCRβ V6 comprises TCRβ V6-4*02, or a variant thereof. In some embodiments, TCRβ V6 comprises TCRβ V6-9*01, or a variant thereof. In some embodiments, TCRβ V6 comprises TCRβ V6-8*01, or a variant thereof. In some embodiments, TCRβ V6 comprises TCRβ V6-5*01, or a variant thereof. In some embodiments, TCRβ V6 comprises TCRβ V6-6*02, or a variant thereof. In some embodiments, TCRβ V6 comprises TCRβ V6-6*01, or a variant thereof. In some embodiments, TCRβ V6 comprises TCRβ V6-2*01, or a variant thereof. In some embodiments, TCRβ V6 comprises TCRβ V6-3*01, or a variant thereof. In some embodiments, TCRβ V6 comprises TCRβ V6-1*01, or a variant thereof.

In some embodiments, TCRβ V6-5*01 is encoded by the nucleic acid sequence of SEQ ID NO: 43, or a sequence having 85%, 90%, 95%, 99% or more identity thereof.

SEQ ID NO: 43
ATGAGCATCGGCCTCCTGTGCTGTGCAGCCTTGTCTCTCCTGTGGGCAG
GTCCAGTGAATGCTGGTGTCACTCAGACCCCAAAATTCCAGGTCCTGAA
GACAGGACAGAGCATGACACTGCAGTGTGCCCAGGATATGAACCATGAA
TACATGTCCTGGTATCGACAAGACCCAGGCATGGGGCTGAGGCTGATTC
ATTACTCAGTTGGTGCTGGTATCACTGACCAAGGAGAAGTCCCCAATGG
CTACAATGTCTCCAGATCAACCACAGAGGATTTCCCGCTCAGGCTGCTG
TCGGCTGCTCCCTCCCAGACATCTGTGTACTTCTGTGCCAGCAGTTACT
C

In some embodiments, TCRβ V6-5*01 comprises the amino acid sequence of SEQ ID NO: 1044, or an amino acid sequence having 85%, 90%, 95%, 99% or more identity thereof.

SEQ ID NO: 1044
MSIGLLCCAALSLLWAGPVNAGVTQTPKFQVLKTGQSMTLQCAQDMNHE
YMSWYRQDPGMGLRLIHYSVGAGITDQGEVPNGYNVSRSTTEDFPLRLL
SAAPSQTSVYFCASSY

In some embodiments, the anti-TCRβV antibody molecule, e.g., anti-TCRβ V6 (e.g., anti-TCRβ V6-5*01) antibody molecule, is a non-murine antibody molecule, e.g., a human or humanized antibody molecule. In some embodiments, the anti-TCRβV antibody molecule, e.g., anti-TCRβ V6 (e.g., anti-TCRβ V6-5*01) antibody molecule is a human antibody molecule. In some embodiments, the anti-TCRβV antibody molecule, e.g., anti-TCRβ V6 (e.g., anti-TCRβ V6-5*01) antibody molecule is a humanized antibody molecule.

In some embodiments, the anti-TCRβV antibody molecule, e.g., anti-TCRβ V6 (e.g., anti-TCRβ V6-5*01) antibody molecule, is isolated or recombinant.

In some embodiments, the anti-TCRβV antibody molecule, e.g., anti-TCRβ V6 (e.g., anti-TCRβ V6-5*01) antibody molecule, comprises at least one antigen-binding region, e.g., a variable region or an antigen-binding fragment thereof, from an antibody described herein, e.g., an antibody chosen from any one of A-H.1 to A-H.85, e.g., A-H.1, A-H.2 or A-H.68, or an antibody described in TABLE 30, or encoded by a nucleotide sequence in TABLE 30, or a sequence substantially identical (e.g., at least 80%, 85%, 90%, 92%, 95%, 97%, 98%, 99% or higher identical) to any of the aforesaid sequences.

In some embodiments, the anti-TCRβV antibody molecule, e.g., anti-TCRβ V6 (e.g., anti-TCRβ V6-5*01) antibody molecule, comprises at least one, two, three or four variable regions from an antibody described herein, e.g., an antibody chosen from any one of A-H.1 to A-H.85, e.g., A-H.1, A-H.2 or A-H.68, or an antibody described in TABLE 30, or encoded by a nucleotide sequence in TABLE 30, or a sequence substantially identical (e.g., at least 80%, 85%, 90%, 92%, 95%, 97%, 98%, 99% or higher identical) to any of the aforesaid sequences.

In some embodiments, the anti-TCRβV antibody molecule, e.g., anti-TCRβ V6 (e.g., anti-TCRβ V6-5*01) antibody molecule, comprises at least one or two heavy chain variable regions from an antibody described herein, e.g., an antibody chosen from any one of A-H.1 to A-H.85, e.g., A-H.1, A-H.2 or A-H.68, or an antibody molecule described in TABLE 30, or encoded by a nucleotide sequence in TABLE 30, or a sequence substantially identical (e.g., at least 80%, 85%, 90%, 92%, 95%, 97%, 98%, 99% or higher identical) to any of the aforesaid sequences.

In some embodiments, the anti-TCRβV antibody molecule comprises a heavy chain variable region (VH) having a consensus sequence of SEQ ID NO: 231 or 3290.

In some embodiments, the anti-TCRβV antibody molecule, e.g., anti-TCRβ V6 (e.g., anti-TCRβ V6-5*01) antibody molecule, comprises at least one or two light chain variable regions from an antibody described herein, e.g., an antibody chosen from any one of A-H.1 to A-H.85, e.g., A-H.1, A-H.2 or A-H.68, or an antibody described in TABLE 30, or encoded by a nucleotide sequence in TABLE 30, or a sequence substantially identical (e.g., at least 80%, 85%, 90%, 92%, 95%, 97%, 98%, 99% or higher identical) to any of the aforesaid sequences.

In some embodiments, the anti-TCRβV antibody molecule comprises a light chain variable region (VL) having a consensus sequence of SEQ ID NO: 230 or 3289.

In some embodiments, the anti-TCRβV antibody molecule, e.g., anti-TCRβ V6 (e.g., anti-TCRβ V6-5*01) antibody molecule, comprises a heavy chain constant region for an IgG4, e.g., a human IgG4. In still another embodiment, the anti-TCRβV antibody molecule, e.g., anti-TCRβ V6 (e.g., anti-TCRβ V6-5*01) antibody molecule includes a heavy chain constant region for an IgG1, e.g., a human IgG1. In one embodiment, the heavy chain constant region comprises an amino sequence set forth in Table 32, or a sequence substantially identical (e.g., at least 80%, 85%, 90%, 92%, 95%, 97%, 98%, 99% or higher identical) thereto.

In some embodiments, the anti-TCRβV antibody molecule, e.g., anti-TCRβ V6 (e.g., anti-TCRβ V6-5*01) antibody molecule, includes a kappa light chain constant region, e.g., a human kappa light chain constant region. In one embodiment, the light chain constant region comprises an amino sequence set forth in Table 32, or a sequence substantially identical (e.g., at least 80%, 85%, 90%, 92%, 95%, 97%, 98%, 99% or higher identical) thereto.

In some embodiments, the anti-TCRβV antibody molecule, e.g., anti-TCRβ V6 (e.g., anti-TCRβ V6-5*01) antibody molecule, includes at least one, two, or three complementarity determining regions (CDRs) from a heavy chain variable region (VH) of an antibody described herein, e.g., an antibody chosen from any one of A-H.1 to A-H.85, e.g., A-H.1, A-H.2 or A-H.68, or an antibody described in TABLE 30, or encoded by a nucleotide sequence in TABLE 30, or a sequence substantially identical (e.g., at least 80%, 85%, 90%, 92%, 95%, 97%, 98%, 99% or higher identical) to any of the aforesaid sequences.

In some embodiments, the anti-TCRβV antibody molecule, e.g., anti-TCRβ V6 (e.g., anti-TCRβ V6-5*01) antibody molecule, includes at least one, two, or three CDRs (or collectively all of the CDRs) from a heavy chain variable region comprising an amino acid sequence shown in TABLE 30, or encoded by a nucleotide sequence shown in TABLE 30. In one embodiment, one or more of the CDRs (or collectively all of the CDRs) have one, two, three, four, five, six or more changes, e.g., amino acid substitutions or deletions, relative to the amino acid sequence shown in TABLE 30, or encoded by a nucleotide sequence shown in TABLE 30.

In some embodiments, the anti-TCRβV antibody molecule, e.g., anti-TCRβ V6 (e.g., anti-TCRβ V6-5*01) antibody molecule, includes at least one, two, or three complementarity determining regions (CDRs) from a light chain variable region of an antibody described herein, e.g., an antibody chosen from any one of A-H.1 to A-H.85, e.g., A-H.1, A-H.2 or A-H.68, or an antibody described in TABLE 30, or encoded by a nucleotide sequence in TABLE 30, or a sequence substantially identical (e.g., at least 80%, 85%, 90%, 92%, 95%, 97%, 98%, 99% or higher identical) to any of the aforesaid sequences.

In some embodiments, the anti-TCRβV antibody molecule, e.g., anti-TCRβ V6 (e.g., anti-TCRβ V6-5*01) antibody molecule, includes at least one, two, or three CDRs (or collectively all of the CDRs) from a light chain variable region comprising an amino acid sequence shown in TABLE 30, or encoded by a nucleotide sequence shown in TABLE 30. In one embodiment, one or more of the CDRs (or collectively all of the CDRs) have one, two, three, four, five, six or more changes, e.g., amino acid substitutions or deletions, relative to the amino acid sequence shown in TABLE 30, or encoded by a nucleotide sequence shown in TABLE 30.

In some embodiments, the anti-TCRβV antibody molecule, e.g., anti-TCRβ V6 (e.g., anti-TCRβ V6-5*01) antibody molecule, includes at least one, two, three, four, five or six CDRs (or collectively all of the CDRs) from a heavy and light chain variable region comprising an amino acid sequence shown in TABLE 30, or encoded by a nucleotide sequence shown in TABLE 30. In one embodiment, one or more of the CDRs (or collectively all of the CDRs) have one, two, three, four, five, six or more changes, e.g., amino acid substitutions or deletions, relative to the amino acid sequence shown in TABLE 30, or encoded by a nucleotide sequence shown in TABLE 30.

In some embodiments, the anti-TCRβV antibody molecule, e.g., anti-TCRβ V6 (e.g., anti-TCRβ V6-5*01) antibody molecule, molecule includes all six CDRs from an antibody described herein, e.g., an antibody chosen from any one of A-H.1 to A-H.85, e.g., A-H.1, A-H.2 or A-H.68, or an antibody described in TABLE 30, or encoded by a nucleotide sequence in TABLE 30, or closely related CDRs, e.g., CDRs which are identical or which have at least one amino acid alteration, but not more than two, three or four alterations (e.g., substitutions, deletions, or insertions, e.g., conservative substitutions). In some embodiments, the anti-TCRβV antibody molecule, e.g., anti-TCRβ V6 (e.g., anti-TCRβ V6-5*01) antibody molecule, may include any CDR described herein.

In some embodiments, the anti-TCRβV antibody molecule, e.g., anti-TCRβ V6 (e.g., anti-TCRβ V6-5*01) antibody molecule includes at least one, two, or three CDRs according to Kabat et al. (e.g., at least one, two, or three CDRs according to the Kabat definition as set out in TABLE 30) from a heavy chain variable region of an antibody described herein, e.g., an antibody chosen from any one of A-H.1 to A-H.85, e.g., A-H.1, A-H.2 or A-H.68, or an antibody described in TABLE 30, or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to one, two, or three CDRs according to Kabat et al. shown in TABLE 30.

In some embodiments, the anti-TCRβV antibody molecule, e.g., anti-TCRβ V6 (e.g., anti-TCRβ V6-5*01) antibody molecule includes at least one, two, or three CDRs according to Kabat et al. (e.g., at least one, two, or three CDRs according to the Kabat definition as set out in TABLE 30) from a light chain variable region of an antibody described herein, e.g., an antibody chosen from any one of A-H.1 to A-H.85, e.g., A-H.1, A-H.2 or A-H.68, or an antibody described in TABLE 30, or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to one, two, or three CDRs according to Kabat et al. shown in TABLE 30.

In some embodiments, the anti-TCRβV antibody molecule, e.g., anti-TCRβ V6 (e.g., anti-TCRβ V6-5*01) antibody molecule, includes at least one, two, three, four, five, or six CDRs according to Kabat et al. (e.g., at least one, two, three, four, five, or six CDRs according to the Kabat definition as set out in TABLE 30) from the heavy and light chain variable regions of an antibody described herein, e.g., an antibody chosen from any one of A-H.1 to A-H.85, e.g., A-H.1, A-H.2 or A-H.68, or an antibody described in TABLE 30, or encoded by a nucleotide sequence in TABLE 30; or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to one, two, three, four, five, or six CDRs according to Kabat et al. shown in TABLE 30.

In some embodiments, the anti-TCRβV antibody molecule, e.g., anti-TCRβ V6 (e.g., anti-TCRβ V6-5*01) antibody molecule, includes all six CDRs according to Kabat et al. (e.g., all six CDRs according to the Kabat definition as set out in TABLE 30) from the heavy and light chain variable regions of an antibody described herein, e.g., an antibody chosen from any one of A-H.1 to A-H.85, e.g., A-H.1, A-H.2 or A-H.68, or an antibody described in TABLE 30, or encoded by a nucleotide sequence in TABLE 30; or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to all six CDRs according to Kabat et al. shown in TABLE 30.

In one embodiment, the anti-TCRβV antibody molecule, e.g., anti-TCRβ V6 (e.g., anti-TCRβ V6-5*01) antibody molecule, may include any CDR described herein.

In some embodiments, the anti-TCRβV antibody molecule, e.g., anti-TCRβ V6 (e.g., anti-TCRβ V6-5*01) antibody molecule, includes at least one, two, or three hypervariable loops that have the same canonical structures as the corresponding hypervariable loop of an antibody described herein, e.g., an antibody chosen from chosen from any one of A-H.1 to A-H.85, e.g., A-H.1, A-H.2 or A-H.68, e.g., the same canonical structures as at least loop 1 and/or loop 2 of the heavy and/or light chain variable domains of an antibody described herein. See, e.g., Chothia et al., (1992) J. Mol. Biol. 227:799-817; Tomlinson et al., (1992) J. Mol. Biol. 227:776-798 for descriptions of hypervariable loop canonical structures. These structures can be determined by inspection of the tables described in these references.

In some embodiments, the anti-TCRβV antibody molecule, e.g., anti-TCRβ V6 (e.g., anti-TCRβ V6-5*01) antibody molecule includes at least one, two, or three CDRs according to Chothia et al. (e.g., at least one, two, or three CDRs according to the Chothia definition as set out in TABLE 30) from a heavy chain variable region of an antibody described herein, e.g., an antibody chosen from any one of A-H.1 to A-H.85, e.g., A-H.1, A-H.2 or A-H.68, or as described in TABLE 30, or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to one, two, or three CDRs according to Chothia et al. shown in TABLE 30.

In some embodiments, the anti-TCRβV antibody molecule, e.g., anti-TCRβ V6 (e.g., anti-TCRβ V6-5*01) antibody molecule includes at least one, two, or three CDRs according to Chothia et al. (e.g., at least one, two, or three CDRs according to the Chothia definition as set out in TABLE 30) from a light chain variable region of an antibody described herein, e.g., an antibody chosen from any one of A-H.1 to A-H.85, e.g., A-H.1, A-H.2 or A-H.68, or an antibody described in TABLE 30, or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to one, two, or three CDRs according to Chothia et al. shown in TABLE 30.

In some embodiments, the anti-TCRβV antibody molecule, e.g., anti-TCRβ V6 (e.g., anti-TCRβ V6-5*01) antibody molecule, includes at least one, two, three, four, five, or six CDRs according to Chothia et al. (e.g., at least one, two, three, four, five, or six CDRs according to the Chothia definition as set out in TABLE 30) from the heavy and light chain variable regions of an antibody described herein, e.g., an antibody chosen from any one of A-H.1 to A-H.85, e.g., A-H.1, A-H.2 or A-H.68, or an antibody described in TABLE 30, or encoded by the nucleotide sequence in TABLE 30; or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to one, two, three, four, five, or six CDRs according to Chothia et al. shown in TABLE 30.

In some embodiments, the anti-TCRβV antibody molecule, e.g., anti-TCRβ V6 (e.g., anti-TCRβ V6-5*01) antibody molecule, includes all six CDRs according to Chothia et al. (e.g., all six CDRs according to the Chothia definition as set out in TABLE 30) from the heavy and light chain variable regions of an antibody described herein, e.g., an antibody chosen from any one of A-H.1 to A-H.85, e.g., A-H.1, A-H.2 or A-H.68, or an antibody described in TABLE 30, or encoded by a nucleotide sequence in TABLE 30; or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to all six CDRs according to Chothia et al. shown in TABLE 30. In one embodiment, the anti-TCRβV antibody molecule, e.g., anti-TCRβ V6 (e.g., anti-TCRβ V6-5*01) antibody molecule, may include any CDR described herein.

In some embodiments, the anti-TCRβV antibody molecule, e.g., anti-TCRβ V6 (e.g., anti-TCRβ V6-5*01) antibody molecule, molecule includes a combination of CDRs or hypervariable loops defined according to Kabat et al., Chothia et al., or as described in TABLE 30.

In some embodiments, the anti-TCRβV antibody molecule, e.g., anti-TCRβ V6 (e.g., anti-TCRβ V6-5*01) antibody molecule, can contain any combination of CDRs or hypervariable loops according to the Kabat and Chothia definitions.

In some embodiments, a combined CDR as set out in TABLE 30 is a CDR that comprises a Kabat CDR and a Chothia CDR.

In some embodiments, the anti-TCRβV antibody molecule, e.g., anti-TCRβ V6 (e.g., anti-TCRβ V6-5*01) antibody molecule, molecule includes a combination of CDRs or hypervariable loops identified as combined CDRs in TABLE 30. In some embodiments, the anti-TCRβV antibody molecule, e.g., anti-TCRβ V6 (e.g., anti-TCRβ V6-5*01) antibody molecule, can contain any combination of CDRs or hypervariable loops according the “combined” CDRs are described in TABLE 30.

In an embodiment, e.g., an embodiment comprising a variable region, a CDR (e.g., a combined CDR, Chothia CDR or Kabat CDR), or other sequence referred to herein, e.g., in TABLE 30, the antibody molecule is a monospecific antibody molecule, a bispecific antibody molecule, a bivalent antibody molecule, a biparatopic antibody molecule, or an antibody molecule that comprises an antigen binding fragment of an antibody, e.g., a half antibody or antigen binding fragment of a half antibody. In certain embodiments, the antibody molecule comprises a multispecific molecule, e.g., a bispecific molecule, e.g., as described herein.

In an embodiment, the anti-TCRβV antibody molecule, e.g., anti-TCR P V6 (e.g., anti-TCR P V6-5*01) antibody molecule includes:
(i) one, two or all of a light chain complementarity determining region 1 (LC CDR1), a light chain complementarity determining region 2 (LC CDR2), and a light chain complementarity determining region 3 (LC CDR3) of SEQ ID NO: 2, SEQ ID NO: 10 or SEQ ID NO: 11, and/or
(ii) one, two or all of a heavy chain complementarity determining region 1 (HC CDR1), heavy chain complementarity determining region 2 (HC CDR2), and a heavy chain complementarity determining region 3 (HC CDR3) of SEQ ID NO: 1 or SEQ ID NO: 9.

In some embodiments, the anti-TCRβV antibody molecule, e.g., anti-TCRβ V6 (e.g., anti-TCRβ V6-5*01) antibody molecule comprises a LC CDR1, LC CDR2, and LC CDR3 of SEQ ID NO: 2, and a HC CDR1, HC CDR2, and HC CDR3 of SEQ ID NO: 1.

In some embodiments the anti-TCRβV antibody molecule, e.g., anti-TCRβ V6 (e.g., anti-TCRβ V6-5*01) antibody molecule comprises a LC CDR1, LC CDR2, and LC CDR3 of SEQ ID NO: 10, and a HC CDR1, HC CDR2, and HC CDR3 of SEQ ID NO: 9.

In some embodiments, the anti-TCR PV antibody molecule, e.g., anti-TCRβ V6 (e.g., anti-TCRβ V6-5*01) antibody molecule comprises a LC CDR1, LC CDR2, and LC CDR3 of SEQ ID NO: 11, and a HC CDR1, HC CDR2, and HC CDR3 of SEQ ID NO: 9.

In an embodiment, the anti-TCRβV antibody molecule, e.g., anti-TCR P V6 (e.g., anti-TCR P V6-5*01) antibody molecule comprises:
(i) a LC CDR1 amino acid sequence of SEQ ID NO: 6, a LC CDR2 amino acid sequence of SEQ ID NO: 7, or a LC CDR3 amino acid sequence of SEQ ID NO: 8; and/or
(ii) a HC CDR1 amino acid sequence of SEQ ID NO: 3, a HC CDR2 amino acid sequence of SEQ ID NO: 4, or a HC CDR3 amino acid sequence of SEQ ID NO: 5.

In some embodiments, the anti-TCRβV antibody molecule, e.g., anti-TCRβ V6 (e.g., anti-TCRβ V6-5*01) antibody molecule comprises:
(i) a light chain variable region (VL) comprising a LC CDR1 amino acid sequence of SEQ ID NO: 6, a LC CDR2 amino acid sequence of SEQ ID NO: 7, or a LC CDR3 amino acid sequence of SEQ ID NO: 8; and/or
(ii) a heavy chain variable region (VH) comprising a HC CDR1 amino acid sequence of SEQ ID NO: 3, a HC CDR2 amino acid sequence of SEQ ID NO: 4, or a HC CDR3 amino acid sequence of SEQ ID NO: 5.

In an embodiment, the anti-TCRβV antibody molecule, e.g., anti-TCR P V6 (e.g., anti-TCR P V6-5*01) antibody molecule comprises:
(i) a LC CDR1 amino acid sequence of SEQ ID NO: 51, a LC CDR2 amino acid sequence of SEQ ID NO: 52, or a LC CDR3 amino acid sequence of SEQ ID NO: 53; and/or
(ii) a HC CDR1 amino acid sequence of SEQ ID NO: 45, a HC CDR2 amino acid sequence of SEQ ID NO: 46, or a HC CDR3 amino acid sequence of SEQ ID NO: 47.

In some embodiments, the anti-TCRβV antibody molecule, e.g., anti-TCRβ V6 (e.g., anti-TCRβV6-5*01) antibody molecule comprises:
(i) a light chain variable region (VL) comprising a LC CDR1 amino acid sequence of SEQ ID NO: 51, a LC CDR2 amino acid sequence of SEQ ID NO: 52, or a LC CDR3 amino acid sequence of SEQ ID NO: 53; and/or
(ii) a heavy chain variable region (VH) comprising a HC CDR1 amino acid sequence of SEQ ID NO: 45, a HC CDR2 amino acid sequence of SEQ ID NO: 46, or a HC CDR3 amino acid sequence of SEQ ID NO: 47.

In an embodiment, the anti-TCRβV antibody molecule, e.g., anti-TCR P V6 (e.g., anti-TCR P V6-5*01) antibody molecule comprises:
(i) a LC CDR1 amino acid sequence of SEQ ID NO: 54, a LC CDR2 amino acid sequence of SEQ ID NO: 55, or a LC CDR3 amino acid sequence of SEQ ID NO: 56; and/or
(ii) a HC CDR1 amino acid sequence of SEQ ID NO: 48, a HC CDR2 amino acid sequence of SEQ ID NO: 49, or a HC CDR3 amino acid sequence of SEQ ID NO: 50.

In some embodiments, the anti-TCRβV antibody molecule, e.g., anti-TCRβ V6 (e.g., anti-TCRβ V6-5*01) antibody molecule comprises:
(i) a light chain variable region (VL) comprising a LC CDR1 amino acid sequence of SEQ ID NO: 54, a LC CDR2 amino acid sequence of SEQ ID NO: 55, or a LC CDR3 amino acid sequence of SEQ ID NO: 56; and/or
(ii) a heavy chain variable region (VH) comprising a HC CDR1 amino acid sequence of SEQ ID NO: 48, a HC CDR2 amino acid sequence of SEQ ID NO: 49, or a HC CDR3 amino acid sequence of SEQ ID NO: 50.

In one embodiment, the light or the heavy chain variable framework (e.g., the region encompassing at least FR1, FR2, FR3, and optionally FR4) of the anti-TCRβV antibody molecule, e.g., anti-TCRβ V6 (e.g., anti-TCRβ V6-5*01) antibody molecule can be chosen from: (a) a light or heavy chain variable framework including at least 80%, 85%, 87% 90%, 92%, 93%, 95%, 97%, 98%, or 100% of the amino acid residues from a human light or heavy chain variable framework, e.g., a light or heavy chain variable framework residue from a human mature antibody, a human germline sequence, or a human consensus sequence; (b) a light or heavy chain variable framework including from 20% to 80%, 40% to 60%, 60% to 90%, or 70% to 95% of the amino acid residues from a human light or heavy chain variable framework, e.g., a light or heavy chain variable framework residue from a human mature antibody, a human germline sequence, or a human consensus sequence; (c) a non-human framework (e.g., a rodent framework); or (d) a non-human framework that has been modified, e.g., to remove antigenic or cytotoxic determinants, e.g., deimmunized, or partially humanized. In one embodiment, the light or heavy chain variable framework region (particularly FR1, FR2 and/or FR3) includes a light or heavy chain variable framework sequence at least 70, 75, 80, 85, 87, 88, 90, 92, 94, 95, 96, 97, 98, 99% identical or identical to the frameworks of a VL or VH segment of a human germline gene.

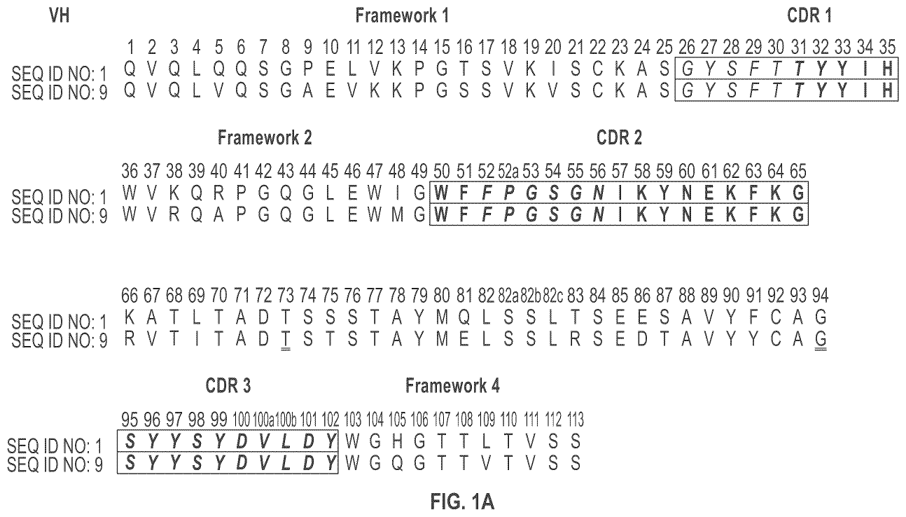

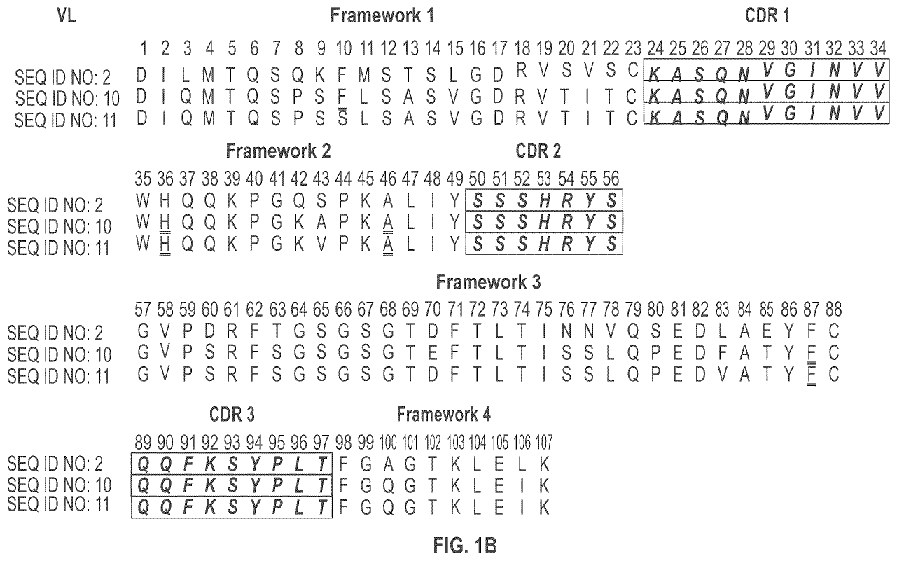

In some embodiments, the anti-TCRβV antibody molecule, e.g., anti-TCRβ V6 (e.g., anti-TCRβ V6-5*01) antibody molecule, comprises a heavy chain variable domain having at least one, two, three, four, five, six, seven, ten, fifteen, twenty or more changes, e.g., amino acid substitutions or deletions, from an amino acid sequence of any one of A-H.1 to A-H.85, e.g., A-H.1, A-H.2 or A-H.68, e.g., the amino acid sequence of the FR region in the entire variable region, e.g., shown in FIG. 1A, or in SEQ ID NO: 9.

Alternatively, or in combination with the heavy chain substitutions described herein, the anti-TCRβV antibody molecule, e.g., anti-TCR P V6 (e.g., anti-TCRβ V6-5*01) antibody molecule, comprises a light chain variable domain having at least one, two, three, four, five, six, seven, ten, fifteen, twenty or more amino acid changes, e.g., amino acid substitutions or deletions, from an amino acid sequence of any one of A-H.1 to A-H.85, e.g., A-H.1, A-H.2 or A-H.68, e.g., the amino acid sequence of the FR region in the entire variable region, e.g., shown in FIG. 1B, or in SEQ ID NO: 10 or SEQ ID NO: 11.

In some embodiments, the anti-TCRβV antibody molecule, e.g., anti-TCRβ V6 (e.g., anti-TCRβ V6-5*01) antibody molecule, includes one, two, three, or four heavy chain framework regions shown in FIG. 1A, or a sequence substantially identical thereto.

In some embodiments, the anti-TCRβV antibody molecule, e.g., anti-TCRβ V6 (e.g., anti-TCRβ V6-5*01) antibody molecule, includes one, two, three, or four light chain framework regions shown in FIG. 1B, or a sequence substantially identical thereto.

In some embodiments, the anti-TCRβV antibody molecule, e.g., anti-TCRβ V6 (e.g., anti-TCRβ V6-5*01) antibody molecule, comprises the light chain framework region 1 of A-H.1 or A-H.2, e.g., as shown in FIG. 1B.

In some embodiments, the anti-TCRβV antibody molecule, e.g., anti-TCRβ V6 (e.g., anti-TCRβ V6-5*01) antibody molecule, comprises the light chain framework region 2 of A-H.1 or A-H.2, e.g., as shown in FIG. 1B.

In some embodiments, the anti-TCRβV antibody molecule, e.g., anti-TCRβ V6 (e.g., anti-TCRβ V6-5*01) antibody molecule, comprises the light chain framework region 3 of A-H.1 or A-H.2, e.g., as shown in FIG. 1B.

In some embodiments, the anti-TCRβV antibody molecule, e.g., anti-TCRβ V6 (e.g., anti-TCRβ V6-5*01) antibody molecule, comprises the light chain framework region 4 of A-H.1 or A-H.2, e.g., as shown in FIG. 1B.

In some embodiments, the anti-TCRβV antibody molecule, e.g., anti-TCRβ V6 (e.g., anti-TCRβ V6-5*01) antibody molecule, comprises a light chain variable domain comprising a framework region, e.g., framework region 1 (FR1), comprising a change, e.g., a substitution (e.g., a conservative substitution) at position 10 according to Kabat numbering. In some embodiments, the FR1 comprises a Phenylalanine at position 10, e.g., a Serine to Phenyalanine substitution. In some embodiments, the substitution is relative to a human germline light chain framework region sequence.

In some embodiments, the anti-TCRβV antibody molecule, e.g., anti-TCRβ V6 (e.g., anti-TCRβ V6-5*01) antibody molecule, comprises a light chain variable domain comprising a framework region, e.g., framework region 2 (FR2), comprising a change, e.g., a substitution (e.g., a conservative substitution) at a position disclosed herein according to Kabat numbering. In some embodiments, FR2 comprises a Histidine at position 36, e.g., a substitution at position 36 according to Kabat numbering, e.g., a Tyrosine to Histidine substitution. In some embodiments, FR2 comprises an Alanine at position 46, e.g., a substitution at position 46 according to Kabat numbering, e.g., an Arginine to Alanine substitution. In some embodiments, the substitution is relative to a human germline light chain framework region sequence.

In some embodiments, the anti-TCRβV antibody molecule, e.g., anti-TCRβ V6 (e.g., anti-TCRβ V6-5*01) antibody molecule, comprises a light chain variable domain comprising a framework region, e.g., framework region 3 (FR3), comprising a change, e.g., a substitution (e.g., a conservative substitution) at a position disclosed herein according to Kabat numbering. In some embodiments, FR3 comprises a Phenyalanine at position 87, e.g., a substitution at position 87 according to Kabat numbering, e.g., a Tyrosine to Phenyalanine substitution. In some embodiments, the substitution is relative to a human germline light chain framework region sequence.

In some embodiments, the anti-TCRβV antibody molecule, e.g., anti-TCRβ V6 (e.g., anti-TCRβ V6-5*01) antibody molecule, comprises a light chain variable domain comprising: (a) a framework region 1 (FR1) comprising a Phenylalanine at position 10, e.g., a substitution at position 10 according to Kabat numbering, e.g., a Serine to Phenyalanine substitution; (b) a framework region 2 (FR2) comprising a Histidine at position 36, e.g., a substitution at position 36 according to Kabat numbering, e.g., a Tyrosine to Histidine substitution, and a Alanine at position 46, e.g., a substitution at position 46 according to Kabat numbering, e.g., a Arginine to Alanine substitution; and (c) a framework region 3 (FR3) comprising a Phenylalanine at position 87, e.g., a substitution at position 87 according to Kabat numbering, e.g., a Tyrosine to Phenyalanine substitution, e.g., as shown in the amino acid sequence of SEQ ID NO: 10. In some embodiments, the substitution is relative to a human germline light chain framework region sequence.

In some embodiments, the anti-TCRβV antibody molecule, e.g., anti-TCRβ V6 (e.g., anti-TCRβ V6-5*01) antibody molecule, comprises a light chain variable domain comprising: (a) a framework region 2 (FR2) comprising a Histidine at position 36, e.g., a substitution at position 36 according to Kabat numbering, e.g., a Tyrosine to Histidine substitution, and a Alanine at position 46, e.g., a substitution at position 46 according to Kabat numbering, e.g., a Arginine to Alanine substitution; and (b) a framework region 3 (FR3) comprising a Phenylalanine at position 87, e.g., a substitution at position 87 according to Kabat numbering, e.g., a Tyrosine to Phenyalanine substitution, e.g., as shown in the amino acid sequence of SEQ ID NO: 11. In some embodiments, the substitution is relative to a human germline light chain framework region sequence.

In some embodiments, the anti-TCRβV antibody molecule, e.g., anti-TCRβ V6 (e.g., anti-TCRβ V6-5*01) antibody molecule, comprises a light chain variable domain comprising: (a) a framework region 1 (FR1) comprising a change, e.g., a substitution (e.g., a conservative substitution) at one or more (e.g., all) positions disclosed herein according to Kabat numbering, (b) a framework region 2 (FR2) comprising a change, e.g., a substitution (e.g., a conservative substitution) at one or more (e.g., all) position disclosed herein according to Kabat numbering and (c) a framework region 3 (FR3) comprising a change, e.g., a substitution (e.g., a conservative substitution) at one or more (e.g., all) position disclosed herein according to Kabat numbering. In some embodiments, the substitution is relative to a human germline light chain framework region sequence.

In some embodiments, the anti-TCRβV antibody molecule, e.g., anti-TCRβ V6 (e.g., anti-TCRβ V6-5*01) antibody molecule, comprises the heavy chain framework region 1 of A-H.1 or A-H.2, e.g., as shown in FIG. 1A.

In some embodiments, the anti-TCRβV antibody molecule, e.g., anti-TCRβ V6 (e.g., anti-TCRβ V6-5*01) antibody molecule, comprises the heavy chain framework region 2 of A-H.1 or A-H.2, e.g., as shown in FIG. 1A

In some embodiments, the anti-TCRβV antibody molecule, e.g., anti-TCRβ V6 (e.g., anti-TCRβ V6-5*01) antibody molecule, comprises the heavy chain framework region 3 of A-H.1 or A-H.2, e.g., as shown in FIG. 1A.

In some embodiments, the anti-TCRβV antibody molecule, e.g., anti-TCRβ V6 (e.g., anti-TCRβ V6-5*01) antibody molecule, comprises the heavy chain framework region 4 of A-H.1 or A-H.2, e.g., as shown in FIG. 1A.

In some embodiments, the anti-TCRβV antibody molecule, e.g., anti-TCRβ V6 (e.g., anti-TCRβ V6-5*01) antibody molecule, comprises a heavy chain variable domain comprising a framework region, e.g., framework region 3 (FR3), comprising a change, e.g., a substitution (e.g., a conservative substitution) at a position disclosed herein according to Kabat numbering. In some embodiments, FR3 comprises a Threonine at position 73, e.g., a substitution at position 73 according to Kabat numbering, e.g., a Glutamic Acid to Threonine substitution. In some embodiments, FR3 comprises a Glycine at position 94, e.g., a substitution at position 94 according to Kabat numbering, e.g., an Arginine to Glycine substitution. In some embodiments, the substitution is relative to a human germline heavy chain framework region sequence.

In some embodiments, the anti-TCRβV antibody molecule, e.g., anti-TCRβ V6 (e.g., anti-TCRβ V6-5*01) antibody molecule, comprises a heavy chain variable domain comprising a framework region 3 (FR3) comprising a Threonine at position 73, e.g., a substitution at position 73 according to Kabat numbering, e.g., a Glutamic Acid to Threonine substitution, and a Glycine at position 94, e.g., a substitution at position 94 according to Kabat numbering, e.g., a Arginine to Glycine substitution, e.g., as shown in the amino acid sequence of SEQ ID NO: 10.

In some embodiments, the anti-TCRβV antibody molecule, e.g., anti-TCRβ V6 (e.g., anti-TCRβ V6-5*01) antibody molecule, comprises the heavy chain framework regions 1-4 of A-H.1 or A-H.2, e.g., SEQ ID NO: 9, or as shown in FIGS. 1A and 1B.

In some embodiments, the anti-TCRβV antibody molecule, e.g., anti-TCRβ V6 (e.g., anti-TCRβ V6-5*01) antibody molecule, comprises the light chain framework regions 1-4 of A-H.1, e.g., SEQ ID NO: 10, or as shown in FIGS. 1A and 1B.

In some embodiments, the anti-TCRβV antibody molecule, e.g., anti-TCRβ V6 (e.g., anti-TCRβ V6-5*01) antibody molecule, comprises the light chain framework regions 1-4 of A-H.2, e.g., SEQ ID NO: 11, or as shown in FIGS. 1A and 1B.

In some embodiments, the anti-TCRβV antibody molecule, e.g., anti-TCRβ V6 (e.g., anti-TCRβ V6-5*01) antibody molecule, comprises the heavy chain framework regions 1-4 of A-H.1, e.g., SEQ ID NO: 9; and the light chain framework regions 1-4 of A-H.1, e.g., SEQ ID NO: 10, or as shown in FIGS. 1A and 1B.

In some embodiments, the anti-TCRβV antibody molecule, e.g., anti-TCRβ V6 (e.g., anti-TCRβ V6-5*01) antibody molecule, comprises the heavy chain framework regions 1-4 of A-H.2, e.g., SEQ ID NO: 9; and the light chain framework regions 1-4 of A-H.2, e.g., SEQ ID NO: 11, or as shown in FIGS. 1A and 1B.

In some embodiments, the heavy or light chain variable domain, or both, of the anti-TCRβV antibody molecule, e.g., anti-TCRβ V6 (e.g., anti-TCRβ V6-5*01) antibody molecule, includes an amino acid sequence, which is substantially identical to an amino acid disclosed herein, e.g., at least 80%, 85%, 90%, 92%, 95%, 97%, 98%, 99% or higher identical to a variable region of an antibody described herein, e.g., an antibody chosen from any one of A-H.1 to A-H.85, e.g., A-H.1, A-H.2 or A-H.68, or as described in TABLE 30, or encoded by the nucleotide sequence in TABLE 30; or which differs at least 1 or 5 residues, but less than 40, 30, 20, or 10 residues, from a variable region of an antibody described herein.

In some embodiments, the anti-TCRβV antibody molecule, e.g., anti-TCRβ V6 (e.g., anti-TCRβ V6-5*01) antibody molecule, comprises at least one, two, three, or four antigen-binding regions, e.g., variable regions, having an amino acid sequence as set forth in TABLE 30, or a sequence substantially identical thereto (e.g., a sequence at least about 85%, 90%, 95%, 99% or more identical thereto, or which differs by no more than 1, 2, 5, 10, or 15 amino acid residues from the sequences shown in TABLE 30. In another embodiment, the anti-TCRβV antibody molecule, e.g., anti-TCRβ V6 (e.g., anti-TCRβ V6-5*01) antibody molecule includes a VH and/or VL domain encoded by a nucleic acid having a nucleotide sequence as set forth in TABLE 30, or a sequence substantially identical thereto (e.g., a sequence at least about 85%, 90%, 95%, 99% or more identical thereto, or which differs by no more than 3, 6, 15, 30, or 45 nucleotides from the sequences shown in TABLE 30.

In some embodiments, the anti-TCRβV antibody molecule, e.g., anti-TCRβ V6 (e.g., anti-TCRβ V6-5*01) antibody molecule, comprises:
a VH domain comprising the amino acid sequence of SEQ ID NO: 9, an amino acid sequence at least about 85%, 90%, 95%, 99% or more identical to the amino acid sequence of SEQ ID NO: 9, or an amino acid sequence which differs by no more than 1, 2, 5, 10, or 15 amino acid residues from the amino acid sequence of SEQ ID NO: 9; and/or
a VL domain comprising the amino acid sequence of SEQ ID NO: 10, an amino acid sequence at least about 85%, 90%, 95%, 99% or more identical to the amino acid sequence of SEQ ID NO: 10, or an amino acid sequence which differs by no more than 1, 2, 5, 10, or 15 amino acid residues from the amino acid sequence of SEQ ID NO: 10.

In some embodiments, the anti-TCRβV antibody molecule, e.g., anti-TCRβ V6 (e.g., anti-TCRβ V6-5*01) antibody molecule, comprises:
a VH domain comprising the amino acid sequence of SEQ ID NO: 9, an amino acid sequence at least about 85%, 90%, 95%, 99% or more identical to the amino acid sequence of SEQ ID NO: 9, or an amino acid sequence which differs by no more than 1, 2, 5, 10, or 15 amino acid residues from the amino acid sequence of SEQ ID NO: 9; and/or
a VL domain comprising the amino acid sequence of SEQ ID NO: 11, an amino acid sequence at least about 85%, 90%, 95%, 99% or more identical to the amino acid sequence of SEQ ID NO: 11, or an amino acid sequence which differs by no more than 1, 2, 5, 10, or 15 amino acid residues from the amino acid sequence of SEQ ID NO: 11.

In some embodiments, the anti-TCRβV antibody molecule, e.g., anti-TCRβ V6 (e.g., anti-TCRβ V6-5*01) antibody molecule is a full antibody or fragment thereof (e.g., a Fab, F(ab′)₂, Fv, or a single chain Fv fragment (scFv)). In some embodiments, the anti-TCRβV antibody molecule, e.g., anti-TCRβ V6 (e.g., anti-TCRβ V6-5*01) antibody molecule is a monoclonal antibody or an antibody with single specificity. In some embodiments, the anti-TCRβV antibody molecule, e.g., anti-TCRβ V6 (e.g., anti-TCRβ V6-5*01) antibody molecule, can also be a humanized, chimeric, camelid, shark, or an in vitro-generated antibody molecule. In some embodiments, the anti-TCRβV antibody molecule, e.g., anti-TCRβ V6 (e.g., anti-TCRβ V6-5*01) antibody molecule, is a humanized antibody molecule. The heavy and light chains of the anti-TCRβV antibody molecule, e.g., anti-TCRβ V6 (e.g., anti-TCRβ V6-5*01) antibody molecule, can be full-length (e.g., an antibody can include at least one, and preferably two, complete heavy chains, and at least one, and preferably two, complete light chains) or can include an antigen-binding fragment (e.g., a Fab, F(ab′)₂, Fv, a single chain Fv fragment, a single domain antibody, a diabody (dAb), a bivalent antibody, or bispecific antibody or fragment thereof, a single domain variant thereof, or a camelid antibody).

In some embodiments, the anti-TCRβV antibody molecule, e.g., anti-TCRβ V6 (e.g., anti-TCRβ V6-5*01) antibody molecule, is in the form of a multispecific molecule, e.g., a bispecific molecule, e.g., as described herein.

In some embodiments, the anti-TCRβV antibody molecule, e.g., anti-TCRβ V6 (e.g., anti-TCRβ V6-5*01) antibody molecule, has a heavy chain constant region (Fc) chosen from, e.g., the heavy chain constant regions of IgG1, IgG2, IgG3, IgG4, IgM, IgA1, IgA2, IgD, and IgE. In some embodiments, the Fc region is chosen from the heavy chain constant regions of IgG1, IgG2, IgG3, and IgG4. In some embodiments, the Fc region is chosen from the heavy chain constant region of IgG1 or IgG2 (e.g., human IgG1, or IgG2). In some embodiments, the heavy chain constant region is human IgG1. In some embodiments, the Fc region comprises a Fc region variant, e.g., as described herein.

In some embodiments, the anti-TCRβV antibody molecule, e.g., anti-TCRβ V6 (e.g., anti-TCRβV6-5*01) antibody molecule, has a light chain constant region chosen from, e.g., the light chain constant regions of kappa or lambda, preferably kappa (e.g., human kappa). In one embodiment, the constant region is altered, e.g., mutated, to modify the properties of the anti-TCRβV antibody molecule, e.g., anti-TCRβ V6 (e.g., anti-TCRβ V6-5*01) antibody molecule (e.g., to increase or decrease one or more of: Fc receptor binding, antibody glycosylation, the number of cysteine residues, effector cell function, or complement function). For example, the constant region is mutated at positions 296 (M to Y), 298 (S to T), 300 (T to E), 477 (H to K) and 478 (N to F) to alter Fc receptor binding (e.g., the mutated positions correspond to positions 132 (M to Y), 134 (S to T), 136 (T to E), 313 (H to K) and 314 (N to F) of SEQ ID NOs: 212 or 214; or positions 135 (M to Y), 137 (S to T), 139 (T to E), 316 (H to K) and 317 (N to F) of SEQ ID NOs: 215, 216, 217 or 218), e.g., relative to human IgG1.

Antibody A-H.1 comprises a heavy chain comprising the amino acid sequence of SEQ ID NO: 3278 and a light chain comprising the amino acid sequence of SEQ ID NO: 72. Antibody A-H.2 comprises a heavy chain comprising the amino acid sequence of SEQ ID NO: 3278 and a light chain comprising the amino acid sequence of SEQ ID NO: 3279. Antibody A-H.68 comprises the amino acid sequence of SEQ ID NO: 1337, or a sequence having at least 85%, 90%, 95%, 96%, 97%, 98%, or 99% identity thereto.

Additional exemplary humanized anti-TCRB V6 antibodies are provided in TABLE 30. In some embodiments, the anti-TCRβ V6 is antibody A, e.g., humanized antibody A (antibody A-H), as provided in TABLE 30. In some embodiments, the anti-TCRβV antibody comprises one or more (e.g., all three) of a LC CDR1, LC CDR2, and LC CDR3 provided in TABLE 30; and/or one or more (e.g., all three) of a HC CDR1, HC CDR2, and HC CDR3 provided in TABLE 30, or a sequence with at least 85%, 90%, 95%, 96%, 97%, 98%, or 99% identity thereto. In some embodiments, antibody A comprises a variable heavy chain (VH) and/or a variable light chain (VL) provided in TABLE 30, or a sequence with at least 85%, 90%, 95%, 96%, 97%, 98%, or 99% identity thereto.

In some embodiments, the anti-TCRβV antibody molecule, e.g., anti-TCRβ V6 (e.g., anti-TCRβ V6-5*01) antibody molecule comprises a VH and/or a VL of an antibody described in Table 1, or a sequence with at least 80%, 85%, 90%, 95%, 96%, 97%, 98%, 99% or more identity thereto. In some embodiments, the anti-TCRβV antibody molecule, e.g., anti-TCR P V6 (e.g., anti-TCR P V6-5*01) antibody molecule comprises a VH of an antibody described in Table 1, or a sequence with at least 80%, 85%, 90%, 95%, 96%, 97%, 98%, 99% or more identity thereto. In some embodiments, the anti-TCRβV antibody molecule, e.g., anti-TCRβ V6 (e.g., anti-TCRβ V6-5*01) antibody molecule comprises a VL of an antibody described in Table 1, or a sequence with at least 80%, 85%, 90%, 95%, 96%, 97%, 98%, 99% or more identity thereto.

In some embodiments, the anti-TCRβV antibody molecule, e.g., anti-TCRβ V6 (e.g., anti-TCRβ V6-5*01) antibody molecule comprises a VH and a VL of an antibody described in Table 1, or a sequence with at least 80%, 85%, 90%, 95%, 96%, 97%, 98%, 99% or more identity thereto.

In some embodiments, the anti-TCRβV antibody molecule, e.g., anti-TCRβ V6 (e.g., anti-TCRβV6-5*01) antibody molecule comprises a VH of A-H.1, A-H.2, A-H.3, A-H.4, A-H.5, A-H.6, A-H.7, A-H.8, A-H.9, A-H.10, A-H.11, A-H.12, A-H.13, A-H.14, A-H.15, A-H.16, A-H.17, A-H.18, A-H.19, A-H.20, A-H.21, A-H.22, A-H.23, A-H.24, A-H.25, A-H.26, A-H.27, A-H.28, A-H.29, A-H.30, A-H.31, A-H.32, A-H.33, A-H.34, A-H.35, A-H.36, A-H.37, A-H.38, A-H.39, A-H.40, A-H.1, A-H.42, A-H.43, A-H.44, A-H.45, A-H.46, A-H.47, A-H.48, A-H.49, A-H.50, A-H.51, A-H.52, A-H.53, A-H.54, A-H.55, A-H.56, A-H.57, A-H.58, A-H.59, A-H.60, A-H.61, A-H.62, A-H.63, A-H.64, A-H.65, A-H.66, A-H.67, A-H.68, A-H.69, A-H.70, A-H.71, A-H.72, A-H.73, A-H.74, A-H.75, A-H.76, A-H.77, A-H.78, A-H.79, A-H.80, A-H.81, A-H.82, A-H.83, A-H.84, or A-H.85, or a sequence with at least 80%, 85%, 90%, 95%, 96%, 97%, 98%, 99% or more identity thereto.

In some embodiments, the anti-TCRβV antibody molecule, e.g., anti-TCRβ V6 (e.g., anti-TCRβ V6-5*01) antibody molecule comprises a VL of A-H.1, A-H.2, A-H.3, A-H.4, A-H.5, A-H.6, A-H.7, A-H.8, A-H.9, A-H.10, A-H.11, A-H.12, A-H.13, A-H.14, A-H.15, A-H.16, A-H.17, A-H.18, A-H.19, A-H.20, A-H.21, A-H.22, A-H.23, A-H.24, A-H.25, A-H.26, A-H.27, A-H.28, A-H.29, A-H.30, A-H.31, A-H.32, A-H.33, A-H.34, A-H.35, A-H.36, A-H.37, A-H.38, A-H.39, A-H.40, A-H.1, A-H.42, A-H.43, A-H.44, A-H.45, A-H.46, A-H.47, A-H.48, A-H.49, A-H.50, A-H.51, A-H.52, A-H.53, A-H.54, A-H.55, A-H.56, A-H.57, A-H.58, A-H.59, A-H.60, A-H.61, A-H.62, A-H.63, A-H.64, A-H.65, A-H.66, A-H.67, A-H.68, A-H.69, A-H.70, A-H.71, A-H.72, A-H.73, A-H.74, A-H.75, A-H.76, A-H.77, A-H.78, A-H.79, A-H.80, A-H.81, A-H.82, A-H.83, A-H.84, or A-H.85, or a sequence with at least 80%, 85%, 90%, 95%, 96%, 97%, 98%, 99% or more identity thereto.

In some embodiments, the anti-TCRβV antibody molecule, e.g., anti-TCRβ V6 (e.g., anti-TCRβ V6-5*01) antibody molecule comprises a VH of A-H.1, A-H.2, A-H.3, A-H.4, A-H.5, A-H.6, A-H.7, A-H.8, A-H.9, A-H.10, A-H.11, A-H.12, A-H.13, A-H.14, A-H.15, A-H.16, A-H.17, A-H.18, A-H.19, A-H.20, A-H.21, A-H.22, A-H.23, A-H.24, A-H.25, A-H.26, A-H.27, A-H.28, A-H.29, A-H.30, A-H.31, A-H.32, A-H.33, A-H.34, A-H.35, A-H.36, A-H.37, A-H.38, A-H.39, A-H.40, A-H.1, A-H.42, A-H.43, A-H.44, A-H.45, A-H.46, A-H.47, A-H.48, A-H.49, A-H.50, A-H.51, A-H.52, A-H.53, A-H.54, A-H.55, A-H.56, A-H.57, A-H.58, A-H.59, A-H.60, A-H.61, A-H.62, A-H.63, A-H.64, A-H.65, A-H.66, A-H.67, A-H.68, A-H.69, A-H.70, A-H.71, A-H.72, A-H.73, A-H.74, A-H.75, A-H.76, A-H.77, A-H.78, A-H.79, A-H.80, A-H.81, A-H.82, A-H.83, A-H.84, or A-H.85, or a sequence with at least 80%, 85%, 90%, 95%, 96%, 97%, 98%, 99% or more identity thereto; and a VL of A-H.1, A-H.2, A-H.3, A-H.4, A-H.5, A-H.6, A-H.7, A-H.8, A-H.9, A-H.10, A-H.11, A-H.12, A-H.13, A-H.14, A-H.15, A-H.16, A-H.17, A-H.18, A-H.19, A-H.20, A-H.21, A-H.22, A-H.23, A-H.24, A-H.25, A-H.26, A-H.27, A-H.28, A-H.29, A-H.30, A-H.31, A-H.32, A-H.33, A-H.34, A-H.35, A-H.36, A-H.37, A-H.38, A-H.39, A-H.40, A-H.1, A-H.42, A-H.43, A-H.44, A-H.45, A-H.46, A-H.47, A-H.48, A-H.49, A-H.50, A-H.51, A-H.52, A-H.53, A-H.54, A-H.55, A-H.56, A-H.57, A-H.58, A-H.59, A-H.60, A-H.61, A-H.62, A-H.63, A-H.64, A-H.65, A-H.66, A-H.67, A-H.68, A-H.69, A-H.70, A-H.71, A-H.72, A-H.73, A-H.74, A-H.75, A-H.76, A-H.77, A-H.78, A-H.79, A-H.80, A-H.81, A-H.82, A-H.83, A-H.84, or A-H.85, or a sequence with at least 80%, 85%, 90%, 95%, 96%, 97%, 98%, 99% or more identity thereto.

TABLE 30
Amino acid and nucleotide sequences for murine, chimeric and humanized antibody
molecules which bind to TCRVB 6, e.g., TCRVB 6-5. The antibody molecules include murine mAb
Antibody A, and humanized mAb Antibody A-H Clones A-H.1 to A-H.85. The amino acid the heavy
and light chain CDRs, and the amino acid and nucleotide sequences of the heavy and light
chain variable regions, and the heavy and light chains are shown.
Antibody A (murine), also referred to as H131, binds to TCRVB 6-5

| | | |
|---|---|---|
| SEQ ID NO: 3 | HC CDR1 (Combined) | GYSFTTYYIH |
| SEQ ID NO: 4 | HC CDR2 (Combined) | WFFPGSGNIKYNEKFKG |
| SEQ ID NO: 5 | HC CDR3 (Combined) | SYYSYDVLDY |
| SEQ ID NO: 45 | HC CDR1 (Kabat) | TYYIH |
| SEQ ID NO: 46 | HC CDR2 (Kabat) | WFFPGSGNIKYNEKFKG |
| SEQ ID NO: 47 | HC CDR3 (Kabat) | SYYSYDVLDY |
| SEQ ID NO: 48 | HC CDR1 (Chothia) | GYSFTTY |
| SEQ ID NO: 49 | HC CDR2 (Chothia) | FPGSGN |
| SEQ ID NO: 50 | HC CDR3 (Chothia) | SYYSYDVLDY |
| SEQ ID NO: 1 | VH | QVQLQQSGPELVKPGTSVKISCKASGYSFTTYYI |
| | | HWVKQRPGQGLEWIGWFFPGSGNIKYNEKFKG |
| | | KATLTADTSSSTAYMQLSSLTSEESAVYFCAGSY |
| | | YSYDVLDYWGHGTTLTVSS |
| SEQ ID NO: 6 | LC CDR1 (Combined) | KASQNVGINVV |
| SEQ ID NO: 7 | LC CDR2 (Combined) | SSSHRYS |
| SEQ ID NO: 8 | LC CDR3 (Combined) | QQFKSYPLT |
| SEQ ID NO: 51 | LC CDR1 (Kabat) | KASQNVGINVV |
| SEQ ID NO: 52 | LC CDR2 (Kabat) | SSSHRYS |
| SEQ ID NO: 53 | LC CDR3 (Kabat) | QQFKSYPLT |
| SEQ ID NO: 54 | LC CDR1 (Chothia) | KASQNVGINVV |
| SEQ ID NO: 55 | LC CDR2 (chothia) | SSSHRYS |
| SEQ ID NO: 56 | LC CDR3 (chothia) | QQFKSYPLT |
| SEQ ID NO: 2 | VL | DILMTQSQKFMSTSLGDRVSVSCKASQNVGINV |
| | | VWHQQKPGQSPKALIYSSSHRYSGVPDRFTGSG |
| | | SGTDFTLTINNVQSEDLAEYFCQQFKSYPLTFGA |
| | | GTKLELK |

Antibody A humanized (A-H antibody)
A-H.1 antibody

| | | |
|---|---|---|
| SEQ ID NO: 3 | HC CDR1 (Combined) | GYSFTTYYIH |
| SEQ ID NO: 4 | HC CDR2 (Combined) | WFFPGSGNIKYNEKFKG |
| SEQ ID NO: 5 | HC CDR3 (Combined) | SYYSYDVLDY |
| SEQ ID NO: 9 | VH | QVQLVQSGAEVKKPGSSVKVSCKASGYSFTTYY |
| | | IHWVRQAPGQGLEWMGWFFPGSGNIKYNEKFK |
| | | GRVTITADTSTSTAYMELSSLRSEDTAVYYCAGS |
| | | YYSYDVLDYWGQGTTVTVSS |
| SEQ ID NO: 12 | DNA VH | CAGGTGCAGCTGGTTCAGTCTGGCGCCGAAGT |
| | | GAAGAAACCTGGCTCCTCCGTGAAGGTGTCCT |
| | | GCAAGGCTTCCGGCTACTCCTTCACCACCTAC |
| | | TACATCCACTGGGTCCGACAGGCCCCTGGACA |
| | | AGGATTGGAATGGATGGGCTGGTTCTTCCCCG |
| | | GCTCCGGCAACATCAAGTACAACGAGAAGTTC |
| | | AAGGGCCGCGTGACCATCACCGCCGACACCTC |
| | | TACCTCTACCGCCTACATGGAACTGTCCAGCC |
| | | TGAGATCTGAGGACACCGCCGTGTACTACTGC |
| | | GCCGGCTCCTACTACTCTTACGACGTGCTGGA |
| | | TTACTGGGGCCAGGGCACCACAGTGACAGTGT |
| | | CCTCT |
| SEQ ID NO: 69 | VH-IgM constant delta | METDTLLLWVLLLWVPGSTGQVQLVQSGAEVK |
| | CDC | KPGSSVKVSCKASGYSFTTYYIHWVRQAPGQGL |
| | | EWMGWFFPGSGNIKYNEKFKGRVTITADTSTST |
| | | AYMELSSLRSEDTAVYYCAGSYYSYDVLDYWG |
| | | QGTTVTVSSGSASAPTLFPLVSCENSPSDTSSVA |
| | | VGCLAQDFLPDSITFSWKYKNNSDISSTRGFPSV |
| | | LRGGKYAATSQVLLPSKDVMQGTDEHVVCKVQ |
| | | HPNGNKEKNVPLPVIAELPPKVSVFVPPRDGFFG |
| | | NPRKSKLICQATGFSPRQIQVSWLREGKQVGSG |
| | | VTTDQVQAEAKESGPTTYKVTSTLTIKESDWLG |
| | | QSMFTCRVDHRGLTFQQNASSMCVPDQDTAIRV |
| | | FAIPPSFASIFLTKSTKLTCLVTDLTTYDSVTISWT |
| | | RQNGEAVKTHTNISESHPNATFSAVGEASICEDD |
| | | WNSGERFTCTVTHTDLASSLKQTISRPKGVALH |
| | | RPDVYLLPPAREQLNLRESATITCLVTGFSPADV |
| | | FVQWMQRGQPLSPEKYVTSAPMPEPQAPGRYF |
| | | AHSILTVSEEEWNTGETYTCVVAHEALPNRVTE |
| | | RTVDKSTGKPTLYNVSLVMSDTAGTCY |
| SEQ ID NO: 70 | VH-IgGA1 | METDTLLLWVLLLWVPGSTGQVQLVQSGAEVK |
| | | KPGSSVKVSCKASGYSFTTYYIHWVRQAPGQGL |
| | | EWMGWFFPGSGNIKYNEKFKGRVTITADTSTST |
| | | AYMELSSLRSEDTAVYYCAGSYYSYDVLDYWG |
| | | QGTTVTVSSASPTSPKVFPLSLCSTQPDGNVVIA |
| | | CLVQGFFPQEPLSVTWSESGQGVTARNFPPSQD |
| | | ASGDLYTTSSQLTLPATQCLAGKSVTCHVKHYT |
| | | NPSQDVTVPCPVPSTPPTPSPSTPPTPSPSCCHPRL |
| | | SLHRPALEDLLLGSEANLTCTLTGLRDASGVTFT |
| | | WTPSSGKSAVQGPPERDLCGCYSVSSVLPGCAE |
| | | PWNHGKTFTCTAAYPESKTPLTATLSKSGNTFRP |
| | | EVHLLPPPSEELALNELVTLTCLARGFSPKDVLV |
| | | RWLQGSQELPREKYLTWASRQEPSQGTTTFAVT |
| | | SILRVAAEDWKKGDTFSCMVGHEALPLAFTQKT |
| | | IDRLAGKPTHVNVSVVMAEVDGTCY |
| SEQ ID NO: 71 | VH-IgGA2 | METDTLLLWVLLLWVPGSTGQVQLVQSGAEVK |
| | | KPGSSVKVSCKASGYSFTTYYIHWVRQAPGQGL |
| | | EWMGWFFPGSGNIKYNEKFKGRVTITADTSTST |
| | | AYMELSSLRSEDTAVYYCAGSYYSYDVLDYWG |
| | | QGTTVTVSSASPTSPKVFPLSLDSTPQDGNVVVA |
| | | CLVQGFFPQEPLSVTWSESGQNVTARNFPPSQD |
| | | ASGDLYTTSSQLTLPATQCPDGKSVTCHVKHYT |
| | | NSSQDVTVPCRVPPPPPCCHPRLSLHRPALEDLL |
| | | LGSEANLTCTLTGLRDASGATFTWTPSSGKSAV |
| | | QGPPERDLCGCYSVSSVLPGCAQPWNHGETFTC |
| | | TAAHPELKTPLTANITKSGNTFRPEVHLLPPPSEE |
| | | LALNELVTLTCLARGFSPKDVLVRWLQGSQELP |
| | | REKYLTWASRQEPSQGTTTYAVTSILRVAAEDW |
| | | KKGETFSCMVGHEALPLAFTQKTIDRMAGKPTH |
| | | INVSVVMAEADGTCY |
| SEQ ID NO: | Heavy chain | METDTLLLWVLLLWVPGSTGQVQLVQSGAEVK |
| 3278 | | KPGSSVKVSCKASGYSFTTYYIHWVRQAPGQGL |
| | | EWMGWFFPGSGNIKYNEKFKGRVTITADTSTST |
| | | AYMELSSLRSEDTAVYYCAGSYYSYDVLDYWG |
| | | QGTTVTVSSASTKGPSVFPLAPSSKSTSGGTAAL |
| | | GCLVKDYFPEPVTVSWNSGALTSGVHTFPAVLQ |
| | | SSGLYSLSSVVTVPSSSLGTQTYICNVNHKPSNT |
| | | KVDKRVEPKSCDKTHTCPPCPAPELLGGPSVFLF |
| | | PPKPKDTLMISRTPEVTCVVVDVSHEDPEVKFN |
| | | WYVDGVEVHNAKTKPREEQYNSTYRVVSVLTV |
| | | LHQDWLNGKEYKCKVSNKALPAPIEKTISKAKG |
| | | QPREPQVYTLPPSREEMTKNQVSLTCLVKGFYPS |
| | | DIAVEWESNGQPENNYKTTPPVLDSDGSFFLYSK |
| | | LTVDKSRWQQGNVFSCSVMHEALHNHYTQKSL |
| | | SLSPGK |
| SEQ ID NO: 6 | LC CDR1 (Combined) | KASQNVGINVV |
| SEQ ID NO: 7 | LC CDR2 (Combined) | SSSHRYS |
| SEQ ID NO: 8 | LC CDR3 (Combined) | QQFKSYPLT |
| SEQ ID NO: 10 | VL | DIQMTQSPSFLSASVGDRVTITCKASQNVGINVV |
| | | WHQQKPGKAPKALIYSSSHRYSGVPSRFSGSGS |
| | | GTEFTLTISSLQPEDFATYFCQQFKSYPLTFGQGT |
| | | KLEIK |
| SEQ ID NO: 13 | DNA VL | GACATCCAGATGACCCAGTCTCCATCCTTCCT |
| | | GTCCGCCTCTGTGGGCGACAGAGTGACCATCA |
| | | CATGCAAGGCCTCTCAGAACGTGGGCATCAAC |
| | | GTCGTGTGGCACCAGCAGAAGCCTGGCAAGG |
| | | CTCCTAAGGCTCTGATCTACTCCTCCAGCCACC |
| | | GGTACTCTGGCGTGCCCTCTAGATTTTCCGGCT |
| | | CTGGCTCTGGCACCGAGTTTACCCTGACAATC |
| | | TCCAGCCTGCAGCCTGAGGACTTCGCCACCTA |
| | | CTTTTGCCAGCAGTTCAAGAGCTACCCTCTGA |
| | | CCTTTGGCCAGGGCACCAAGCTGGAAATCAAG |
| SEQ ID NO: 72 | VL and kappa constant | METDTLLLWVLLLWVPGSTGDIQMTQSPSFLSA |
| | region/light chain | SVGDRVTITCKASQNVGINVVWHQQKPGKAPK |
| | | ALIYSSSHRYSGVPSRFSGSGSGTEFTLTISSLQPE |
| | | DFATYFCQQFKSYPLTFGQGTKLEIKRTVAAPSV |
| | | FIFPPSDEQLKSGTASVVCLLNNFYPREAKVQWK |
| | | VDNALQSGNSQESVTEQDSKDSTYSLSSTLTLSK |
| | | ADYEKHKVYACEVTHQGLSSPVTKSFNRGEC |

A-H.2 antibody

| | | |
|---|---|---|
| SEQ ID NO: 3 | HC CDR1 (Combined) | GYSFTTYYIH |
| SEQ ID NO: 4 | HC CDR2 (Combined) | WFFPGSGNIKYNEKFKG |
| SEQ ID NO: 5 | HC CDR3 (Combined) | SYYSYDVLDY |
| SEQ ID NO: 9 | VH | QVQLVQSGAEVKKPGSSVKVSCKASGYSFTTYY |
| | | IHWVRQAPGQGLEWMGWFFPGSGNIKYNEKFK |
| | | GRVTITADTSTSTAYMELSSLRSEDTAVYYCAGS |
| | | YYSYDVLDYWGQGTTVTVSS |
| SEQ ID NO: 12 | DNA VH | CAGGTGCAGCTGGTTCAGTCTGGCGCCGAAGT |
| | | GAAGAAACCTGGCTCCTCCGTGAAGGTGTCCT |
| | | GCAAGGCTTCCGGCTACTCCTTCACCACCTAC |
| | | TACATCCACTGGGTCCGACAGGCCCCTGGACA |
| | | AGGATTGGAATGGATGGGCTGGTTCTTCCCCG |
| | | GCTCCGGCAACATCAAGTACAACGAGAAGTTC |
| | | AAGGGCCGCGTGACCATCACCGCCGACACCTC |
| | | TACCTCTACCGCCTACATGGAACTGTCCAGCC |
| | | TGAGATCTGAGGACACCGCCGTGTACTACTGC |
| | | GCCGGCTCCTACTACTCTTACGACGTGCTGGA |
| | | TTACTGGGGCCAGGGCACCACAGTGACAGTGT |
| | | CCTCT |
| SEQ ID NO: | Heavy chain | METDTLLLWVLLLWVPGSTGQVQLVQSGAEVK |
| 3278 | | KPGSSVKVSCKASGYSFTTYYIHWVRQAPGQGL |
| | | EWMGWFFPGSGNIKYNEKFKGRVTITADTSTST |
| | | AYMELSSLRSEDTAVYYCAGSYYSYDVLDYWG |
| | | QGTTVTVSSASTKGPSVFPLAPSSKSTSGGTAAL |
| | | GCLVKDYFPEPVTVSWNSGALTSGVHTFPAVLQ |
| | | SSGLYSLSSVVTVPSSSLGTQTYICNVNHKPSNT |
| | | KVDKRVEPKSCDKTHTCPPCPAPELLGGPSVFLF |
| | | PPKPKDTLMISRTPEVTCVVVDVSHEDPEVKFN |
| | | WYVDGVEVHNAKTKPREEQYNSTYRVVSVLTV |
| | | LHQDWLNGKEYKCKVSNKALPAPIEKTISKAKG |
| | | QPREPQVYTLPPSREEMTKNQVSLTCLVKGFYPS |
| | | DIAVEWESNGQPENNYKTTPPVLDSDGSFFLYSK |
| | | LTVDKSRWQQGNVFSCSVMHEALHNHYTQKSL |
| | | SLSPGK |
| SEQ ID NO: 6 | LC CDR1 (Combined) | KASQNVGINVV |
| SEQ ID NO: 7 | LC CDR2 (Combined) | SSSHRYS |
| SEQ ID NO: 8 | LC CDR3 (Combined) | QQFKSYPLT |
| SEQ ID NO: 11 | VL | DIQMTQSPSSLSASVGDRVTITCKASQNVGINVV |
| | | WHQQKPGKVPKALIYSSSHRYSGVPSRFSGSGS |
| | | GTDFTLTISSLQPEDVATYFCQQFKSYPLTFGQG |
| | | TKLEIK |
| SEQ ID NO: 14 | DNA VL | GACATCCAGATGACCCAGTCTCCATCCTCTCT |
| | | GTCCGCCTCTGTGGGCGACAGAGTGACCATCA |
| | | CATGCAAGGCCTCTCAGAACGTGGGCATCAAC |
| | | GTCGTGTGGCACCAGCAGAAACCTGGCAAGGT |
| | | GCCCAAGGCTCTGATCTACTCCTCCAGCCACA |
| | | GATACTCCGGCGTGCCCTCTAGATTCTCCGGC |
| | | TCTGGCTCTGGCACCGACTTTACCCTGACAAT |
| | | CTCCAGCCTGCAGCCTGAGGACGTGGCCACCT |
| | | ACTTTTGCCAGCAGTTCAAGAGCTACCCTCTG |
| | | ACCTTTGGCCAGGGCACCAAGCTGGAAATCAA |
| | | G |
| SEQ ID NO: | Light chain | METDTLLLWVLLLWVPGSTGDIQMTQSPSSLSA |
| 3279 | | SVGDRVTITCKASQNVGINVVWHQQKPGKVPK |
| | | ALIYSSSHRYSGVPSRFSGSGSGTDFTLTISSLQPE |
| | | DVATYFCQQFKSYPLTFGQGTKLEIKRTVAAPSV |
| | | FIFPPSDEQLKSGTASVVCLLNNFYPREAKVQWK |
| | | VDNALQSGNSQESVTEQDSKDSTYSLSSTLTLSK |
| | | ADYEKHKVYACEVTHQGLSSPVTKSFNRGEC |

A-H.3 antibody

| | | |
|---|---|---|
| SEQ ID NO: 80 | VH + VL | QVQLVQSGAEVKKPGSSVKVSCKASGTDFKLTY |
| | | IHWVRQAPGQGLEWMGRVSPGSGNTKYNEKFK |
| | | GRVTITADTSTSTAYMELSSLRSEDTAVYYCAGS |
| | | YYSYDVLDYWGQGTTVTVSSGGGGSGGGGSGG |
| | | GGSGGGGSDIQMTQSPSFLSASVGDRVTITCKAS |
| | | QNVEDRVAWYQQKPGKAPKALIYSSSHRYKGV |
| | | PSRFSGSGSGTEFTLTISSLQPEDFATYFCQQFKS |
| | | YPLTFGQGTKLEIK |
| SEQ ID NO: 81 | VL | DIQMTQSPSFLSASVGDRVTITCKASQNVEDRVA |
| | | WYQQKPGKAPKALIYSSSHRYKGVPSRFSGSGS |
| | | GTEFTLTISSLQPEDFATYFCQQFKSYPLTFGQGT |
| | | KLEIK |
| SEQ ID NO: 82 | VH | QVQLVQSGAEVKKPGSSVKVSCKASGTDFKLTY |
| | | IHWVRQAPGQGLEWMGRVSPGSGNTKYNEKFK |
| | | GRVTITADTSTSTAYMELSSLRSEDTAVYYCAGS |
| | | YYSYDVLDYWGQGTTVTVSS |

A-H.4

| | | |
|---|---|---|
| SEQ ID NO: 83 | VH+VL | QVQLVQSGAEVKKPGSSVKVSCKASGTDFDKIY |
| | | IHWVRQAPGQGLEWMGRISAGSGNVKYNEKFK |
| | | GRVTITADTSTSTAYMELSSLRSEDTAVYYCAGS |
| | | YYSYDVLDYWGQGTTVTVSSGGGGSGGGGSGG |
| | | GGSGGGGSDIQMTQSPSFLSASVGDRVTITCKAS |
| | | QNVEDRVAWYQQKPGKAPKALIYSSSHRYKGV |
| | | PSRFSGSGSGTEFTLTISSLQPEDFATYFCQQFKS |
| | | YPLTFGQGTKLEIK |
| SEQ ID NO: 84 | VI | DIQMTQSPSFLSASVGDRVTITCKASQNVEDRVA |
| | | WYQQKPGKAPKALIYSSSHRYKGVPSRFSGSGS |
| | | GTEFTLTISSLQPEDFATYFCQQFKSYPLTFGQGT |
| | | KLEIK |
| SEQ ID NO: 85 | VH | QVQLVQSGAEVKKPGSSVKVSCKASGTDFDKIY |
| | | IHWVRQAPGQGLEWMGRISAGSGNVKYNEKFK |
| | | GRVTITADTSTSTAYMELSSLRSEDTAVYYCAGS |
| | | YYSYDVLDYWGQGTTVTVSS |

A-H.5

| | | |
|---|---|---|
| SEQ ID NO: 86 | VH+VL | QVQLVQSGAEVKKPGSSVKVSCKASGHDFRDF |
| | | YIHWVRQAPGQGLEWMGRVYPGSGSYRYNEKF |
| | | KGRVTITADTSTSTAYMELSSLRSEDTAVYYCA |
| | | GSYYSYDVLDYWGQGTTVTVSSGGGGSGGGGS |
| | | GGGGSGGGGSDIQMTQSPSFLSASVGDRVTITCK |
| | | ASQNVDDRVAWYQQKPGKAPKALIYSSSHRYK |
| | | GVPSRFSGSGSGTEFTLTISSLQPEDFATYFCQQF |
| | | KSYPLTFGQGTKLEIK |
| SEQ ID NO: 87 | VL | DIQMTQSPSFLSASVGDRVTITCKASQNVDDRV |
| | | AWYQQKPGKAPKALIYSSSHRYKGVPSRFSGSG |
| | | SGTEFTLTISSLQPEDFATYFCQQFKSYPLTFGQG |
| | | TKLEIK |
| SEQ ID NO: 88 | VH | QVQLVQSGAEVKKPGSSVKVSCKASGHDFRDF |
| | | YIHWVRQAPGQGLEWMGRVYPGSGSYRYNEKF |
| | | KGRVTITADTSTSTAYMELSSLRSEDTAVYYCA |
| | | GSYYSYDVLDYWGQGTTVTVSS |

A-H.6

| | | |
|---|---|---|
| SEQ ID NO: 89 | VH + VL | QVQLVQSGAEVKKPGSSVKVSCKASGHDFKLT |
| | | YIHWVRQAPGQGLEWMGRISAGSGNVKYNEKF |
| | | KGRVTITADTSTSTAYMELSSLRSEDTAVYYCA |
| | | GSYYSYDVLDYWGQGTTVTVSSGGGGSGGGGS |
| | | GGGGSGGGGSDIQMTQSPSFLSASVGDRVTITCK |
| | | ASQNVDNRVAWYQQKPGKAPKALIYSSSHRYK |
| | | GVPSRFSGSGSGTEFTLTISSLQPEDFATYFCQQF |
| | | KSYPLTFGQGTKLEIK |
| SEQ ID NO: 90 | VL | DIQMTQSPSFLSASVGDRVTITCKASQNVDNRV |
| | | AWYQQKPGKAPKALIYSSSHRYKGVPSRFSGSG |
| | | SGTEFTLTISSLQPEDFATYFCQQFKSYPLTFGQG |
| | | TKLEIK |
| SEQ ID NO: 91 | VH | QVQLVQSGAEVKKPGSSVKVSCKASGHDFKLT |
| | | YIHWVRQAPGQGLEWMGRISAGSGNVKYNEKF |
| | | KGRVTITADTSTSTAYMELSSLRSEDTAVYYCA |
| | | GSYYSYDVLDYWGQGTTVTVSS |

A-H.7

| | | |
|---|---|---|
| SEQ ID NO: 92 | VH + VL | QVQLVQSGAEVKKPGSSVKVSCKASGTDFKLTY |
| | | IHWVRQAPGQGLEWMGRIFPGSGNVKYNEKFK |
| | | GRVTITADTSTSTAYMELSSLRSEDTAVYYCAGS |
| | | YYSYDVLDYWGQGTTVTVSSGGGGSGGGGSGG |
| | | GGSGGGGSDIQMTQSPSFLSASVGDRVTITCKAS |
| | | QNVENKVAWHQQKPGKAPKALIYSSSHRYKGV |
| | | PSRFSGSGSGTEFTLTISSLQPEDFATYFCQQFKS |
| | | YPLTFGQGTKLEIK |
| SEQ ID NO: 93 | VL | DIQMTQSPSFLSASVGDRVTITCKASQNVENKVA |
| | | WHQQKPGKAPKALIYSSSHRYKGVPSRFSGSGS |
| | | GTEFTLTISSLQPEDFATYFCQQFKSYPLTFGQGT |
| | | KLEIK |
| SEQ ID NO: 94 | VH | QVQLVQSGAEVKKPGSSVKVSCKASGTDFKLTY |
| | | IHWVRQAPGQGLEWMGRIFPGSGNVKYNEKFK |
| | | GRVTITADTSTSTAYMELSSLRSEDTAVYYCAGS |
| | | YYSYDVLDYWGQGTTVTVSS |

A-H.8

| | | |
|---|---|---|
| SEQ ID NO: 95 | VH + VL | QVQLVQSGAEVKKPGSSVKVSCKASGTDFDKIY |
| | | IHWVRQAPGQGLEWMGRIFAGSGNTKYNEKFK |
| | | GRVTITADTSTSTAYMELSSLRSEDTAVYYCAGS |
| | | YYSYDVLDYWGQGTTVTVSSGGGGSGGGGSGG |
| | | GGSGGGGSDIQMTQSPSFLSASVGDRVTITCKAS |
| | | QNVDDRVAWYQQKPGKAPKALIYSSSHRYKGV |
| | | PSRFSGSGSGTEFTLTISSLQPEDFATYFCQQFKS |
| | | YPLTFGQGTKLEIK |
| SEQ ID NO: 96 | VL | DIQMTQSPSFLSASVGDRVTITCKASQNVDDRV |
| | | AWYQQKPGKAPKALIYSSSHRYKGVPSRFSGSG |
| | | SGTEFTLTISSLQPEDFATYFCQQFKSYPLTFGQG |
| | | TKLEIK |
| SEQ ID NO: 97 | VH | QVQLVQSGAEVKKPGSSVKVSCKASGTDFDKIY |
| | | IHWVRQAPGQGLEWMGRIFAGSGNTKYNEKFK |
| | | GRVTITADTSTSTAYMELSSLRSEDTAVYYCAGS |
| | | YYSYDVLDYWGQGTTVTVSS |

A-H.9

| | | |
|---|---|---|
| SEQ ID NO: 98 | VH + VL | QVQLVQSGAEVKKPGSSVKVSCKASGHDFDKF |
| | | YIHWVRQAPGQGLEWMGRVSAGSGNVKYNEK |
| | | FKGRVTITADTSTSTAYMELSSLRSEDTAVYYCA |
| | | GSYYSYDVLDYWGQGTTVTVSSGGGGSGGGGS |
| | | GGGGSGGGGSDIQMTQSPSFLSASVGDRVTITCK |
| | | ASQNVGNRVAWYQQKPGKAPKALIYSSSHRYS |
| | | GVPSRFSGSGSGTEFTLTISSLQPEDFATYFCQQF |
| | | KSYPLTFGQGTKLEIK |
| SEQ ID NO: 99 | VL | DIQMTQSPSFLSASVGDRVTITCKASQNVGNRV |
| | | AWYQQKPGKAPKALIYSSSHRYSGVPSRFSGSG |
| | | SGTEFTLTISSLQPEDFATYFCQQFKSYPLTFGQG |
| | | TKLEIK |
| SEQ ID NO: 100 | VH | QVQLVQSGAEVKKPGSSVKVSCKASGHDFDKF |
| | | YIHWVRQAPGQGLEWMGRVSAGSGNVKYNEK |
| | | FKGRVTITADTSTSTAYMELSSLRSEDTAVYYCA |
| | | GSYYSYDVLDYWGQGTTVTVSS |

A-H.10

| | | |
|---|---|---|
| SEQ ID NO: 101 | VH + VL | QVQLVQSGAEVKKPGSSVKVSCKASGHDFDKF |
| | | YIHWVRQAPGQGLEWMGRIFAGSGNVKYNEKF |
| | | KGRVTITADTSTSTAYMELSSLRSEDTAVYYCA |
| | | GSYYSYDVLDYWGQGTTVTVSSGGGGSGGGGS |
| | | GGGGSGGGGSDIQMTQSPSFLSASVGDRVTITCK |
| | | ASQNVGDRVAWYQQKPGKAPKALIYSSSHRYK |
| | | GVPSRFSGSGSGTEFTLTISSLQPEDFATYFCQQF |
| | | KSYPLTFGQGTKLEIKs |
| SEQ ID NO: 102 | VL | DIQMTQSPSFLSASVGDRVTITCKASQNVGDRV |
| | | AWYQQKPGKAPKALIYSSSHRYKGVPSRFSGSG |
| | | SGTEFTLTISSLQPEDFATYFCQQFKSYPLTFGQG |
| | | TKLEIK |
| SEQ ID NO: 103 | VH | QVQLVQSGAEVKKPGSSVKVSCKASGHDFDKF |
| | | YIHWVRQAPGQGLEWMGRIFAGSGNVKYNEKF |
| | | KGRVTITADTSTSTAYMELSSLRSEDTAVYYCA |
| | | GSYYSYDVLDYWGQGTTVTVSS |

A-H.11

| | | |
|---|---|---|
| SEQ ID NO: 104 | VH + VL | QVQLVQSGAEVKKPGSSVKVSCKASGTDFKLTY |
| | | IHWVRQAPGQGLEWMGRVSPGSGNVKYNEKFK |
| | | GRVTITADTSTSTAYMELSSLRSEDTAVYYCAGS |
| | | YYSYDVLDYWGQGTTVTVSSGGGGSGGGGSGG |
| | | GGSGGGGSDIQMTQSPSFLSASVGDRVTITCKAS |
| | | QNVGDRVAWYQQKPGKAPKALIYSSSHRYKGV |
| | | PSRFSGSGSGTEFTLTISSLQPEDFATYFCQQFKS |
| | | YPLTFGQGTKLEIK |
| SEQ ID NO: 105 | VL | DIQMTQSPSFLSASVGDRVTITCKASQNVGDRV |
| | | AWYQQKPGKAPKALIYSSSHRYKGVPSRFSGSG |
| | | SGTEFTLTISSLQPEDFATYFCQQFKSYPLTFGQG |
| | | TKLEIK |
| SEQ ID NO: 106 | VH | QVQLVQSGAEVKKPGSSVKVSCKASGTDFKLTY |
| | | IHWVRQAPGQGLEWMGRVSPGSGNVKYNEKFK |
| | | GRVTITADTSTSTAYMELSSLRSEDTAVYYCAGS |
| | | YYSYDVLDYWGQGTTVTVSS |

A-H.12

| | | |
|---|---|---|
| SEQ ID NO: 107 | VH + VL | QVQLVQSGAEVKKPGSSVKVSCKASGTDFDKIY |
| | | IHWVRQAPGQGLEWMGRVSAGSGNTKYNEKFK |
| | | GRVTITADTSTSTAYMELSSLRSEDTAVYYCAGS |
| | | YYSYDVLDYWGQGTTVTVSSGGGGSGGGGSGG |
| | | GGSGGGGSDIQMTQSPSFLSASVGDRVTITCKAS |
| | | QNVGNRVAWYQQKPGKAPKALIYSSSHRYKGV |
| | | PSRFSGSGSGTEFTLTISSLQPEDFATYFCQQFKS |
| | | YPLTFGQGTKLEIK |
| SEQ ID NO: 108 | VL | DIQMTQSPSFLSASVGDRVTITCKASQNVGNRV |
| | | AWYQQKPGKAPKALIYSSSHRYKGVPSRFSGSG |
| | | SGTEFTLTISSLQPEDFATYFCQQFKSYPLTFGQG |
| | | TKLEIK |
| SEQ ID NO: 109 | VH | QVQLVQSGAEVKKPGSSVKVSCKASGTDFDKIY |
| | | IHWVRQAPGQGLEWMGRVSAGSGNTKYNEKFK |
| | | GRVTITADTSTSTAYMELSSLRSEDTAVYYCAGS |
| | | YYSYDVLDYWGQGTTVTVSS |

A-H.13 (also referred to as A-H.69)

| | | |
|---|---|---|
| SEQ ID NO: 110 | VH + VL | QVQLVQSGAEVKKPGSSVKVSCKASGTDFKLTY |
| | | IHWVRQAPGQGLEWMGRIFPGSGNVKYNEKFK |
| | | GRVTITADTSTSTAYMELSSLRSEDTAVYYCAGS |
| | | YYSYDVLDYWGQGTTVTVSSGGGGSGGGGSGG |
| | | GGSGGGGSDIQMTQSPSFLSASVGDRVTITCKAS |
| | | QNVDNRVAWYQQKPGKAPKALIYSSSHRYKGV |
| | | PSRFSGSGSGTEFTLTISSLQPEDFATYFCQQFKS |
| | | YPLTFGQGTKLEIK |
| SEQ ID NO: 111 | VL | DIQMTQSPSFLSASVGDRVTITCKASQNVDNRV |
| | | AWYQQKPGKAPKALIYSSSHRYKGVPSRFSGSG |
| | | SGTEFTLTISSLQPEDFATYFCQQFKSYPLTFGQG |
| | | TKLEIK |
| SEQ ID NO: 112 | VH | QVQLVQSGAEVKKPGSSVKVSCKASGTDFKLTY |
| | | IHWVRQAPGQGLEWMGRIFPGSGNVKYNEKFK |
| | | GRVTITADTSTSTAYMELSSLRSEDTAVYYCAGS |
| | | YYSYDVLDYWGQGTTVTVSS |

A-H.14

| | | |
|---|---|---|
| SEQ ID NO: 113 | VH + VL | QVQLVQSGAEVKKPGSSVKVSCKASGTDFDKIY |
| | | IHWVRQAPGQGLEWMGRISAGSGNTKYNEKFK |
| | | GRVTITADTSTSTAYMELSSLRSEDTAVYYCAGS |
| | | YYSYDVLDYWGQGTTVTVSSGGGGSGGGGSGG |
| | | GGSGGGGSDIQMTQSPSFLSASVGDRVTITCKAS |
| | | QNVDDRVAWYQQKPGKAPKALIYSSSHRYKGV |
| | | PSRFSGSGSGTEFTLTISSLQPEDFATYFCQQFKS |
| | | YPLTFGQGTKLEIK |
| SEQ ID NO: 114 | VL | DIQMTQSPSFLSASVGDRVTITCKASQNVDDRV |
| | | AWYQQKPGKAPKALIYSSSHRYKGVPSRFSGSG |
| | | SGTEFTLTISSLQPEDFATYFCQQFKSYPLTFGQG |
| | | TKLEIK |
| SEQ ID NO: 115 | VH | QVQLVQSGAEVKKPGSSVKVSCKASGTDFDKIY |
| | | IHWVRQAPGQGLEWMGRISAGSGNTKYNEKFK |
| | | GRVTITADTSTSTAYMELSSLRSEDTAVYYCAGS |
| | | YYSYDVLDYWGQGTTVTVSS |

A-H.15

| | | |
|---|---|---|
| SEQ ID NO: 116 | VH + VL | QVQLVQSGAEVKKPGSSVKVSCKASGTDFRLTY |
| | | IHWVRQAPGQGLEWMGRVSPGSGNTKYNEKFK |
| | | GRVTITADTSTSTAYMELSSLRSEDTAVYYCAGS |
| | | YYSYDVLDYWGQGTTVTVSSGGGGSGGGGSGG |
| | | GGSGGGGSDIQMTQSPSFLSASVGDRVTITCKAS |
| | | QNVDNKVAWHQQKPGKAPKALIYSSSHRYKGV |
| | | PSRFSGSGSGTEFTLTISSLQPEDFATYFCQQFKS |
| | | YPLTFGQGTKLEIK |
| SEQ ID NO: 117 | VL | DIQMTQSPSFLSASVGDRVTITCKASQNVDNKV |
| | | AWHQQKPGKAPKALIYSSSHRYKGVPSRFSGSG |
| | | SGTEFTLTISSLQPEDFATYFCQQFKSYPLTFGQG |
| | | TKLEIK |
| SEQ ID NO: 118 | VH | QVQLVQSGAEVKKPGSSVKVSCKASGTDFRLTY |
| | | IHWVRQAPGQGLEWMGRVSPGSGNTKYNEKFK |
| | | GRVTITADTSTSTAYMELSSLRSEDTAVYYCAGS |
| | | YYSYDVLDYWGQGTTVTVSS |

A-H.16

| | | |
|---|---|---|
| SEQ ID NO: 119 | VH + VL | QVQLVQSGAEVKKPGSSVKVSCKASGGTFRLTY |
| | | IHWVRQAPGQGLEWMGRVYPGSGNTKYNEKFK |
| | | GRVTITADTSTSTAYMELSSLRSEDTAVYYCAGS |
| | | YYSYDVLDYWGQGTTVTVSSGGGGSGGGGSGG |
| | | GGSGGGGSDIQMTQSPSFLSASVGDRVTITCKAS |
| | | QNVDDRVAWYQQKPGKAPKALIYSSSHRYKGV |
| | | PSRFSGSGSGTEFTLTISSLQPEDFATYFCQQFKS |
| | | YPLTFGQGTKLEIK |
| SEQ ID NO: 120 | VL | DIQMTQSPSFLSASVGDRVTITCKASQNVDDRV |
| | | AWYQQKPGKAPKALIYSSSHRYKGVPSRFSGSG |
| | | SGTEFTLTISSLQPEDFATYFCQQFKSYPLTFGQG |
| | | TKLEIK |
| SEQ ID NO: 121 | VH | QVQLVQSGAEVKKPGSSVKVSCKASGGTFRLTY |
| | | IHWVRQAPGQGLEWMGRVYPGSGNTKYNEKFK |
| | | GRVTITADTSTSTAYMELSSLRSEDTAVYYCAGS |
| | | YYSYDVLDYWGQGTTVTVSS |

A-H.17

| | | |
|---|---|---|
| SEQ ID NO: 122 | VH+VL | QVQLVQSGAEVKKPGSSVKVSCKASGTDFRLTY |
| | | IHWVRQAPGQGLEWMGRIFPGSGNTKYNEKFK |
| | | GRVTITADTSTSTAYMELSSLRSEDTAVYYCAGS |
| | | YYSYDVLDYWGQGTTVTVSSGGGGSGGGGSGG |
| | | GGSGGGGSDIQMTQSPSFLSASVGDRVTITCKAS |
| | | QNVDDRVAWYQQKPGKAPKALIYSSSHRYKGV |
| | | PSRFSGSGSGTEFTLTISSLQPEDFATYFCQQFKS |
| | | YPLTFGQGTKLEIK |
| SEQ ID NO: 123 | VL | DIQMTQSPSFLSASVGDRVTITCKASQNVDDRV |
| | | AWYQQKPGKAPKALIYSSSHRYKGVPSRFSGSG |
| | | SGTEFTLTISSLQPEDFATYFCQQFKSYPLTFGQG |
| | | TKLEIK |
| SEQ ID NO: 124 | VH | QVQLVQSGAEVKKPGSSVKVSCKASGTDFRLTY |
| | | IHWVRQAPGQGLEWMGRIFPGSGNTKYNEKFK |
| | | GRVTITADTSTSTAYMELSSLRSEDTAVYYCAGS |
| | | YYSYDVLDYWGQGTTVTVSS |

A-H.18

| | | |
|---|---|---|
| SEQ ID NO: 125 | VH + VL | QVQLVQSGAEVKKPGSSVKVSCKASGTDFKLTY |
| | | IHWVRQAPGQGLEWMGRIFPGSGNVKYNEKFK |
| | | GRVTITADTSTSTAYMELSSLRSEDTAVYYCAGS |
| | | YYSYDVLDYWGQGTTVTVSSGGGGSGGGGSGG |
| | | GGSGGGGSDIQMTQSPSFLSASVGDRVTITCKAS |
| | | QNVEDRVAWYQQKPGKAPKALIYSSSHRYKGV |
| | | PSRFSGSGSGTEFTLTISSLQPEDFATYFCQQFKS |
| | | YPLTFGQGTKLEIK |
| SEQ ID NO: 126 | VL | DIQMTQSPSFLSASVGDRVTITCKASQNVEDRVA |
| | | WYQQKPGKAPKALIYSSSHRYKGVPSRFSGSGS |
| | | GTEFTLTISSLQPEDFATYFCQQFKSYPLTFGQGT |
| | | KLEIK |
| SEQ ID NO: 127 | VH | QVQLVQSGAEVKKPGSSVKVSCKASGTDFKLTY |
| | | IHWVRQAPGQGLEWMGRIFPGSGNVKYNEKFK |
| | | GRVTITADTSTSTAYMELSSLRSEDTAVYYCAGS |
| | | YYSYDVLDYWGQGTTVTVSS |

A-H.19

| | | |
|---|---|---|
| SEQ ID NO: 128 | VH + VL | QVQLVQSGAEVKKPGSSVKVSCKASGGTFRLTY |
| | | IHWVRQAPGQGLEWMGRISAGSGNVKYNEKFK |
| | | GRVTITADTSTSTAYMELSSLRSEDTAVYYCAGS |
| | | YYSYDVLDYWGQGTTVTVSSGGGGSGGGGSGG |
| | | GGSGGGGSDIQMTQSPSFLSASVGDRVTITCKAS |
| | | QNVGDRVAWYQQKPGKAPKALIYSSSHRYKGV |
| | | PSRFSGSGSGTEFTLTISSLQPEDFATYFCQQFKS |
| | | YPLTFGQGTKLEIK |
| SEQ ID NO: 129 | VL | DIQMTQSPSFLSASVGDRVTITCKASQNVGDRV |
| | | AWYQQKPGKAPKALIYSSSHRYKGVPSRFSGSG |
| | | SGTEFTLTISSLQPEDFATYFCQQFKSYPLTFGQG |
| | | TKLEIK |
| SEQ ID NO: 130 | VH | QVQLVQSGAEVKKPGSSVKVSCKASGGTFRLTY |
| | | IHWVRQAPGQGLEWMGRISAGSGNVKYNEKFK |
| | | GRVTITADTSTSTAYMELSSLRSEDTAVYYCAGS |
| | | YYSYDVLDYWGQGTTVTVSS |

A-H.20

| | | |
|---|---|---|
| SEQ ID NO: 131 | VH + VL | QVQLVQSGAEVKKPGSSVKVSCKASGGTFDKT |
| | | YIHWVRQAPGQGLEWMGRISAGSGNTKYNEKF |
| | | KGRVTITADTSTSTAYMELSSLRSEDTAVYYCA |
| | | GSYYSYDVLDYWGQGTTVTVSSGGGGSGGGGS |
| | | GGGGSGGGGSDIQMTQSPSFLSASVGDRVTITCK |
| | | ASQNVDDRVAWYQQKPGKAPKALIYSSSHRYK |
| | | GVPSRFSGSGSGTEFTLTISSLQPEDFATYFCQQF |
| | | KSYPLTFGQGTKLEIK |
| SEQ ID NO: 132 | VL | DIQMTQSPSFLSASVGDRVTITCKASQNVDDRV |
| | | AWYQQKPGKAPKALIYSSSHRYKGVPSRFSGSG |
| | | SGTEFTLTISSLQPEDFATYFCQQFKSYPLTFGQG |
| | | TKLEIK |
| SEQ ID NO: 133 | VH | QVQLVQSGAEVKKPGSSVKVSCKASGGTFDKT |
| | | YIHWVRQAPGQGLEWMGRISAGSGNTKYNEKF |
| | | KGRVTITADTSTSTAYMELSSLRSEDTAVYYCA |
| | | GSYYSYDVLDYWGQGTTVTVSS |

A-H.21

| | | |
|---|---|---|
| SEQ ID NO: 134 | VH + VL | QVQLVQSGAEVKKPGSSVKVSCKASGHDFDKF |
| | | YIHWVRQAPGQGLEWMGRISAGSGNTKYNEKF |
| | | KGRVTITADTSTSTAYMELSSLRSEDTAVYYCA |
| | | GSYYSYDVLDYWGQGTTVTVSSGGGGSGGGGS |
| | | GGGGSGGGGSDIQMTQSPSFLSASVGDRVTITCK |
| | | ASQNVDDRVAWYQQKPGKAPKALIYSSSHRYK |
| | | GVPSRFSGSGSGTEFTLTISSLQPEDFATYFCQQF |
| | | KSYPLTFGQGTKLEIK |
| SEQ ID NO: 135 | VL | DIQMTQSPSFLSASVGDRVTITCKASQNVDDRV |
| | | AWYQQKPGKAPKALIYSSSHRYKGVPSRFSGSG |
| | | SGTEFTLTISSLQPEDFATYFCQQFKSYPLTFGQG |
| | | TKLEIK |
| SEQ ID NO: 136 | VH | QVQLVQSGAEVKKPGSSVKVSCKASGHDFDKF |
| | | YIHWVRQAPGQGLEWMGRISAGSGNTKYNEKF |
| | | KGRVTITADTSTSTAYMELSSLRSEDTAVYYCA |
| | | GSYYSYDVLDYWGQGTTVTVSS |

A-H.22

| | | |
|---|---|---|
| SEQ ID NO: 137 | VH + VL | QVQLVQSGAEVKKPGSSVKVSCKASGTDFKLTY |
| | | IHWVRQAPGQGLEWMGRISAGSGNVKYNEKFK |
| | | GRVTITADTSTSTAYMELSSLRSEDTAVYYCAGS |
| | | YYSYDVLDYWGQGTTVTVSSGGGGSGGGGSGG |
| | | GGSGGGGSDIQMTQSPSFLSASVGDRVTITCKAS |
| | | QNVDNKVAWHQQKPGKAPKALIYSSSHRYKGV |
| | | PSRFSGSGSGTEFTLTISSLQPEDFATYFCQQFKS |
| | | YPLTFGQGTKLEIK |
| SEQ ID NO: 138 | VL | DIQMTQSPSFLSASVGDRVTITCKASQNVDNKV |
| | | AWHQQKPGKAPKALIYSSSHRYKGVPSRFSGSG |
| | | SGTEFTLTISSLQPEDFATYFCQQFKSYPLTFGQG |
| | | TKLEIK |
| SEQ ID NO: 139 | VH | QVQLVQSGAEVKKPGSSVKVSCKASGTDFKLTY |
| | | IHWVRQAPGQGLEWMGRISAGSGNVKYNEKFK |
| | | GRVTITADTSTSTAYMELSSLRSEDTAVYYCAGS |
| | | YYSYDVLDYWGQGTTVTVSS |

A-H.23

| | | |
|---|---|---|
| SEQ ID NO: 140 | VH + VL | QVQLVQSGAEVKKPGSSVKVSCKASGHDFRLTY |
| | | IHWVRQAPGQGLEWMGRISAGSGNVKYNEKFK |
| | | GRVTITADTSTSTAYMELSSLRSEDTAVYYCAGS |
| | | YYSYDVLDYWGQGTTVTVSSGGGGSGGGGSGG |
| | | GGSGGGGSDIQMTQSPSFLSASVGDRVTITCKAS |
| | | QNVADRVAWYQQKPGKAPKALIYSSSHRYKGV |
| | | PSRFSGSGSGTEFTLTISSLQPEDFATYFCQQFKS |
| | | YPLTFGQGTKLEIK |
| SEQ ID NO: 141 | VL | DIQMTQSPSFLSASVGDRVTITCKASQNVADRV |
| | | AWYQQKPGKAPKALIYSSSHRYKGVPSRFSGSG |
| | | SGTEFTLTISSLQPEDFATYFCQQFKSYPLTFGQG |
| | | TKLEIK |
| SEQ ID NO: 142 | VH | QVQLVQSGAEVKKPGSSVKVSCKASGHDFRLTY |
| | | IHWVRQAPGQGLEWMGRISAGSGNVKYNEKFK |
| | | GRVTITADTSTSTAYMELSSLRSEDTAVYYCAGS |
| | | YYSYDVLDYWGQGTTVTVSS |

A-H.24

| | | |
|---|---|---|
| SEQ ID NO: 143 | VH + VL | QVQLVQSGAEVKKPGSSVKVSCKASGHDFHLW |
| | | YIHWVRQAPGQGLEWMGRVSAGSGNVKYNEK |
| | | FKGRVTITADTSTSTAYMELSSLRSEDTAVYYCA |
| | | GSYYSYDVLDYWGQGTTVTVSSGGGGSGGGGS |
| | | GGGGSGGGGSDIQMTQSPSFLSASVGDRVTITCK |
| | | ASQNVDNKVAWHQQKPGKAPKALIYSSSHRYK |
| | | GVPSRFSGSGSGTEFTLTISSLQPEDFATYFCQQF |
| | | KSYPLTFGQGTKLEIK |
| SEQ ID NO: 144 | VL | DIQMTQSPSFLSASVGDRVTITCKASQNVDNKV |
| | | AWHQQKPGKAPKALIYSSSHRYKGVPSRFSGSG |
| | | SGTEFTLTISSLQPEDFATYFCQQFKSYPLTFGQG |
| | | TKLEIK |
| SEQ ID NO: 145 | VH | QVQLVQSGAEVKKPGSSVKVSCKASGHDFHLW |
| | | YIHWVRQAPGQGLEWMGRVSAGSGNVKYNEK |
| | | FKGRVTITADTSTSTAYMELSSLRSEDTAVYYCA |
| | | GSYYSYDVLDYWGQGTTVTVSS |

A-H.25

| | | |
|---|---|---|
| SEQ ID NO: 146 | VH + VL | QVQLVQSGAEVKKPGSSVKVSCKASGHDFHLW |
| | | YIHWVRQAPGQGLEWMGRVFAGSGNTKYNEKF |
| | | KGRVTITADTSTSTAYMELSSLRSEDTAVYYCA |
| | | GSYYSYDVLDYWGQGTTVTVSSGGGGSGGGGS |
| | | GGGGSGGGGSDIQMTQSPSFLSASVGDRVTITCK |
| | | ASQNVEDKVAWYQQKPGKAPKALIYSSSHRYK |
| | | GVPSRFSGSGSGTEFTLTISSLQPEDFATYFCQQF |
| | | KSYPLTFGQGTKLEIK |
| SEQ ID NO: 147 | VL | DIQMTQSPSFLSASVGDRVTITCKASQNVEDKVA |
| | | WYQQKPGKAPKALIYSSSHRYKGVPSRFSGSGS |
| | | GTEFTLTISSLQPEDFATYFCQQFKSYPLTFGQGT |
| | | KLEIK |
| SEQ ID NO: 148 | VH | QVQLVQSGAEVKKPGSSVKVSCKASGHDFHLW |
| | | YIHWVRQAPGQGLEWMGRVFAGSGNTKYNEKF |
| | | KGRVTITADTSTSTAYMELSSLRSEDTAVYYCA |
| | | GSYYSYDVLDYWGQGTTVTVSS |

A-H.26

| | | |
|---|---|---|
| SEQ ID NO: 149 | VH + VL | QVQLVQSGAEVKKPGSSVKVSCKASGTDFKLTY |
| | | IHWVRQAPGQGLEWMGRIFPGSGNTKYNEKFK |
| | | GRVTITADTSTSTAYMELSSLRSEDTAVYYCAGS |
| | | YYSYDVLDYWGQGTTVTVSSGGGGSGGGGSGG |
| | | GGSGGGGSDIQMTQSPSFLSASVGDRVTITCKAS |
| | | QNVDDRVAWYQQKPGKAPKALIYSSSHRYKGV |
| | | PSRFSGSGSGTEFTLTISSLQPEDFATYFCQQFKS |
| | | YPLTFGQGTKLEIK |
| SEQ ID NO: 150 | VL | DIQMTQSPSFLSASVGDRVTITCKASQNVDDRV |
| | | AWYQQKPGKAPKALIYSSSHRYKGVPSRFSGSG |
| | | SGTEFTLTISSLQPEDFATYFCQQFKSYPLTFGQG |
| | | TKLEIK |
| SEQ ID NO: 151 | VH | QVQLVQSGAEVKKPGSSVKVSCKASGTDFKLTY |
| | | IHWVRQAPGQGLEWMGRIFPGSGNTKYNEKFK |
| | | GRVTITADTSTSTAYMELSSLRSEDTAVYYCAGS |
| | | YYSYDVLDYWGQGTTVTVSS |

A-H.27

| | | |
|---|---|---|
| SEQ ID NO: 153 | VH + VL | QVQLVQSGAEVKKPGSSVKVSCKASGTDFKLTY |
| | | IHWVRQAPGQGLEWMGRVSAGSGNVKYNEKF |
| | | KGRVTITADTSTSTAYMELSSLRSEDTAVYYCA |
| | | GSYYSYDVLDYWGQGTTVTVSSGGGGSGGGGS |
| | | GGGGSGGGGSDIQMTQSPSFLSASVGDRVTITCK |
| | | ASQNVGNRVAWYQQKPGKAPKALIYSSSHRYK |
| | | GVPSRFSGSGSGTEFTLTISSLQPEDFATYFCQQF |
| | | KSYPLTFGQGTKLEIK |
| SEQ ID NO: 154 | VL | DIQMTQSPSFLSASVGDRVTITCKASQNVGNRV |
| | | AWYQQKPGKAPKALIYSSSHRYKGVPSRFSGSG |
| | | SGTEFTLTISSLQPEDFATYFCQQFKSYPLTFGQG |
| | | TKLEIK |
| SEQ ID NO: 155 | VH | QVQLVQSGAEVKKPGSSVKVSCKASGTDFKLTY |
| | | IHWVRQAPGQGLEWMGRVSAGSGNVKYNEKF |
| | | KGRVTITADTSTSTAYMELSSLRSEDTAVYYCA |
| | | GSYYSYDVLDYWGQGTTVTVSS |

A-H.28

| | | |
|---|---|---|
| SEQ ID NO: 156 | VH + VL | QVQLVQSGAEVKKPGSSVKVSCKASGTDFKLTY |
| | | IHWVRQAPGQGLEWMGRISPGSGNTKYNEKFK |
| | | GRVTITADTSTSTAYMELSSLRSEDTAVYYCAGS |
| | | YYSYDVLDYWGQGTTVTVSSGGGGSGGGGSGG |
| | | GGSGGGGSDIQMTQSPSFLSASVGDRVTITCKAS |
| | | QNVGDRVAWYQQKPGKAPKALIYSSSHRYKGV |
| | | PSRFSGSGSGTEFTLTISSLQPEDFATYFCQQFKS |
| | | YPLTFGQGTKLEIK |
| SEQ ID NO: 157 | VL | DIQMTQSPSFLSASVGDRVTITCKASQNVGDRV |
| | | AWYQQKPGKAPKALIYSSSHRYKGVPSRFSGSG |
| | | SGTEFTLTISSLQPEDFATYFCQQFKSYPLTFGQG |
| | | TKLEIK |
| SEQ ID NO: 158 | VH | QVQLVQSGAEVKKPGSSVKVSCKASGTDFKLTY |
| | | IHWVRQAPGQGLEWMGRISPGSGNTKYNEKFK |
| | | GRVTITADTSTSTAYMELSSLRSEDTAVYYCAGS |
| | | YYSYDVLDYWGQGTTVTVSS |

A-H.29

| | | |
|---|---|---|
| SEQ ID NO: 159 | VH + VL | QVQLVQSGAEVKKPGSSVKVSCKASGHDFHLW |
| | | YIHWVRQAPGQGLEWMGRISPGSGNVKYNEKF |
| | | KGRVTITADTSTSTAYMELSSLRSEDTAVYYCA |
| | | GSYYSYDVLDYWGQGTTVTVSSGGGGSGGGGS |
| | | GGGGSGGGGSDIQMTQSPSFLSASVGDRVTITCK |
| | | ASQNVGDRVAWHQQKPGKAPKALIYSSSHRYK |
| | | GVPSRFSGSGSGTEFTLTISSLQPEDFATYFCQQF |
| | | KSYPLTFGQGTKLEIK |
| SEQ ID NO: 160 | VL | DIQMTQSPSFLSASVGDRVTITCKASQNVGDRV |
| | | AWHQQKPGKAPKALIYSSSHRYKGVPSRFSGSG |
| | | SGTEFTLTISSLQPEDFATYFCQQFKSYPLTFGQG |
| | | TKLEIK |
| SEQ ID NO: 161 | VH | QVQLVQSGAEVKKPGSSVKVSCKASGHDFHLW |
| | | YIHWVRQAPGQGLEWMGRISPGSGNVKYNEKF |
| | | KGRVTITADTSTSTAYMELSSLRSEDTAVYYCA |
| | | GSYYSYDVLDYWGQGTTVTVSS |

A-H.31

| | | |
|---|---|---|
| SEQ ID NO: 162 | VH + VL | QVQLVQSGAEVKKPGSSVKVSCKASGHDFKLT |
| | | YIHWVRQAPGQGLEWMGRISAGSGNVKYNEKF |
| | | KGRVTITADTSTSTAYMELSSLRSEDTAVYYCA |
| | | GSYYSYDVLDYWGQGTTVTVSSGGGGSGGGGS |
| | | GGGGSGGGGSDIQMTQSPSFLSASVGDRVTITCK |
| | | ASQNVDDRVAWYQQKPGKAPKALIYSSSHRYK |
| | | GVPSRFSGSGSGTEFTLTISSLQPEDFATYFCQQF |
| | | KSYPLTFGQGTKLEIK |
| SEQ ID NO: 163 | VL | DIQMTQSPSFLSASVGDRVTITCKASQNVDDRV |
| | | AWYQQKPGKAPKALIYSSSHRYKGVPSRFSGSG |
| | | SGTEFTLTISSLQPEDFATYFCQQFKSYPLTFGQG |
| | | TKLEIK |
| SEQ ID NO: 164 | VH | QVQLVQSGAEVKKPGSSVKVSCKASGHDFKLT |
| | | YIHWVRQAPGQGLEWMGRISAGSGNVKYNEKF |
| | | KGRVTITADTSTSTAYMELSSLRSEDTAVYYCA |
| | | GSYYSYDVLDYWGQGTTVTVSS |

A-H.31

| | | |
|---|---|---|
| SEQ ID NO: 165 | VH + VL | QVQLVQSGAEVKKPGSSVKVSCKASGTDFHLW |
| | | YIHWVRQAPGQGLEWMGRVFAGSGSYRYNEKF |
| | | KGRVTITADTSTSTAYMELSSLRSEDTAVYYCA |
| | | GSYYSYDVLDYWGQGTTVTVSSGGGGSGGGGS |
| | | GGGGSGGGGSDIQMTQSPSFLSASVGDRVTITCK |
| | | ASQNVDDRVAWYQQKPGKAPKALIYSSSHRYK |
| | | GVPSRFSGSGSGTEFTLTISSLQPEDFATYFCQQF |
| | | KSYPLTFGQGTKLEIK |
| SEQ ID NO: 166 | VL | DIQMTQSPSFLSASVGDRVTITCKASQNVDDRV |
| | | AWYQQKPGKAPKALIYSSSHRYKGVPSRFSGSG |
| | | SGTEFTLTISSLQPEDFATYFCQQFKSYPLTFGQG |
| | | TKLEIK |
| SEQ ID NO: 167 | VH | QVQLVQSGAEVKKPGSSVKVSCKASGTDFHLW |
| | | YIHWVRQAPGQGLEWMGRVFAGSGSYRYNEKF |
| | | KGRVTITADTSTSTAYMELSSLRSEDTAVYYCA |
| | | GSYYSYDVLDYWGQGTTVTVSS |

A-H.32

| | | |
|---|---|---|
| SEQ ID NO: 168 | VH + VL | QVQLVQSGAEVKKPGSSVKVSCKASGTDFDKIY |
| | | IHWVRQAPGQGLEWMGRISAGSGNTKYNEKFK |
| | | GRVTITADTSTSTAYMELSSLRSEDTAVYYCAGS |
| | | YYSYDVLDYWGQGTTVTVSSGGGGSGGGGSGG |
| | | GGSGGGGSDIQMTQSPSFLSASVGDRVTITCKAS |
| | | QNVADRVAWYQQKPGKAPKALIYSSSHRYKGV |
| | | PSRFSGSGSGTEFTLTISSLQPEDFATYFCQQFKS |
| | | YPLTFGQGTKLEIK |
| SEQ ID NO: 169 | VL | DIQMTQSPSFLSASVGDRVTITCKASQNVADRV |
| | | AWYQQKPGKAPKALIYSSSHRYKGVPSRFSGSG |
| | | SGTEFTLTISSLQPEDFATYFCQQFKSYPLTFGQG |
| | | TKLEIK |
| SEQ ID NO: 170 | VH | QVQLVQSGAEVKKPGSSVKVSCKASGTDFDKIY |
| | | IHWVRQAPGQGLEWMGRISAGSGNTKYNEKFK |
| | | GRVTITADTSTSTAYMELSSLRSEDTAVYYCAGS |
| | | YYSYDVLDYWGQGTTVTVSS |

A-H.33

| | | |
|---|---|---|
| SEQ ID NO: 171 | VH + VL | QVQLVQSGAEVKKPGSSVKVSCKASGTDFKLTY |
| | | IHWVRQAPGQGLEWMGRISAGSGNTKYNEKFK |
| | | GRVTITADTSTSTAYMELSSLRSEDTAVYYCAGS |
| | | YYSYDVLDYWGQGTTVTVSSGGGGSGGGGSGG |
| | | GGSGGGGSDIQMTQSPSFLSASVGDRVTITCKAS |
| | | QNVEDRVAWYQQKPGKAPKALIYSSSHRYKGV |
| | | PSRFSGSGSGTEFTLTISSLQPEDFATYFCQQFKS |
| | | YPLTFGQGTKLEIK |
| SEQ ID NO: 172 | VL | DIQMTQSPSFLSASVGDRVTITCKASQNVEDRVA |
| | | WYQQKPGKAPKALIYSSSHRYKGVPSRFSGSGS |
| | | GTEFTLTISSLQPEDFATYFCQQFKSYPLTFGQGT |
| | | KLEIK |
| SEQ ID NO: 173 | VH | QVQLVQSGAEVKKPGSSVKVSCKASGTDFKLTY |
| | | IHWVRQAPGQGLEWMGRISAGSGNTKYNEKFK |
| | | GRVTITADTSTSTAYMELSSLRSEDTAVYYCAGS |
| | | YYSYDVLDYWGQGTTVTVSS |

A-H.34

| | | |
|---|---|---|
| SEQ ID NO: 174 | VH + VL | QVQLVQSGAEVKKPGSSVKVSCKASGTDFRLTY |
| | | IHWVRQAPGQGLEWMGRISPGSGNTKYNEKFK |
| | | GRVTITADTSTSTAYMELSSLRSEDTAVYYCAGS |
| | | YYSYDVLDYWGQGTTVTVSSGGGGSGGGGSGG |
| | | GGSGGGGSDIQMTQSPSFLSASVGDRVTITCKAS |
| | | QNVGNRVAWYQQKPGKAPKALIYSSSHRYKGV |
| | | PSRFSGSGSGTEFTLTISSLQPEDFATYFCQQFKS |
| | | YPLTFGQGTKLEIK |
| SEQ ID NO: 175 | VL | DIQMTQSPSFLSASVGDRVTITCKASQNVGNRV |
| | | AWYQQKPGKAPKALIYSSSHRYKGVPSRFSGSG |
| | | SGTEFTLTISSLQPEDFATYFCQQFKSYPLTFGQG |
| | | TKLEIK |
| SEQ ID NO: 176 | VH | QVQLVQSGAEVKKPGSSVKVSCKASGTDFRLTY |
| | | IHWVRQAPGQGLEWMGRISPGSGNTKYNEKFK |
| | | GRVTITADTSTSTAYMELSSLRSEDTAVYYCAGS |
| | | YYSYDVLDYWGQGTTVTVSS |

A-H.35

| | | |
|---|---|---|
| SEQ ID NO: 177 | VH + VL | QVQLVQSGAEVKKPGSSVKVSCKASGHDFDKT |
| | | YIHWVRQAPGQGLEWMGRVSAGSGNVKYNEK |
| | | FKGRVTITADTSTSTAYMELSSLRSEDTAVYYCA |
| | | GSYYSYDVLDYWGQGTTVTVSSGGGGSGGGGS |
| | | GGGGSGGGGSDIQMTQSPSFLSASVGDRVTITCK |
| | | ASQNVEDRVAWYQQKPGKAPKALIYSSSHRYK |
| | | GVPSRFSGSGSGTEFTLTISSLQPEDFATYFCQQF |
| | | KSYPLTFGQGTKLEIK |
| SEQ ID NO: 178 | VL | DIQMTQSPSFLSASVGDRVTITCKASQNVEDRVA |
| | | WYQQKPGKAPKALIYSSSHRYKGVPSRFSGSGS |
| | | GTEFTLTISSLQPEDFATYFCQQFKSYPLTFGQGT |
| | | KLEIK |
| SEQ ID NO: 179 | VH | QVQLVQSGAEVKKPGSSVKVSCKASGHDFDKT |
| | | YIHWVRQAPGQGLEWMGRVSAGSGNVKYNEK |
| | | FKGRVTITADTSTSTAYMELSSLRSEDTAVYYCA |
| | | GSYYSYDVLDYWGQGTTVTVSS |

A-H.36

| | | |
|---|---|---|
| SEQ ID NO: 180 | VH + VL | QVQLVQSGAEVKKPGSSVKVSCKASGHDFKLT |
| | | YIHWVRQAPGQGLEWMGRVSPGSGNTKYNEKF |
| | | KGRVTITADTSTSTAYMELSSLRSEDTAVYYCA |
| | | GSYYSYDVLDYWGQGTTVTVSSGGGGSGGGGS |
| | | GGGGSGGGGSDIQMTQSPSFLSASVGDRVTITCK |
| | | ASQNVEDRVAWHQQKPGKAPKALIYSSSHRYK |
| | | GVPSRFSGSGSGTEFTLTISSLQPEDFATYFCQQF |
| | | KSYPLTFGQGTKLEIK |
| SEQ ID NO: 181 | VL | DIQMTQSPSFLSASVGDRVTITCKASQNVEDRVA |
| | | WHQQKPGKAPKALIYSSSHRYKGVPSRFSGSGS |
| | | GTEFTLTISSLQPEDFATYFCQQFKSYPLTFGQGT |
| | | KLEIK |
| SEQ ID NO: 182 | VH | QVQLVQSGAEVKKPGSSVKVSCKASGHDFKLT |
| | | YIHWVRQAPGQGLEWMGRVSPGSGNTKYNEKF |
| | | KGRVTITADTSTSTAYMELSSLRSEDTAVYYCA |
| | | GSYYSYDVLDYWGQGTTVTVSS |

A-H.37

| | | |
|---|---|---|
| SEQ ID NO: 183 | VH+VL | QVQLVQSGAEVKKPGSSVKVSCKASGHDFDKT |
| | | YIHWVRQAPGQGLEWMGRIYPGSGNVKYNEKF |
| | | KGRVTITADTSTSTAYMELSSLRSEDTAVYYCA |
| | | GSYYSYDVLDYWGQGTTVTVSSGGGGSGGGGS |
| | | GGGGSGGGGSDIQMTQSPSFLSASVGDRVTITCK |
| | | ASQNVADRVAWYQQKPGKAPKALIYSSSHRYK |
| | | GVPSRFSGSGSGTEFTLTISSLQPEDFATYFCQQF |
| | | KSYPLTFGQGTKLEIK |
| SEQ ID NO: 184 | VL | DIQMTQSPSFLSASVGDRVTITCKASQNVADRV |
| | | AWYQQKPGKAPKALIYSSSHRYKGVPSRFSGSG |
| | | SGTEFTLTISSLQPEDFATYFCQQFKSYPLTFGQG |
| | | TKLEIK |
| SEQ ID NO: 185 | VH | QVQLVQSGAEVKKPGSSVKVSCKASGHDFDKT |
| | | YIHWVRQAPGQGLEWMGRIYPGSGNVKYNEKF |
| | | KGRVTITADTSTSTAYMELSSLRSEDTAVYYCA |
| | | GSYYSYDVLDYWGQGTTVTVSS |

A-H.38

| | | |
|---|---|---|
| SEQ ID NO: 186 | VH + VL | QVQLVQSGAEVKKPGSSVKVSCKASGTDFDKT |
| | | YIHWVRQAPGQGLEWMGRISAGSGNVKYNEKF |
| | | KGRVTITADTSTSTAYMELSSLRSEDTAVYYCA |
| | | GSYYSYDVLDYWGQGTTVTVSSGGGGSGGGGS |
| | | GGGGSGGGGSDIQMTQSPSFLSASVGDRVTITCK |
| | | ASQNVDDRVAWYQQKPGKAPKALIYSSSHRYK |
| | | GVPSRFSGSGSGTEFTLTISSLQPEDFATYFCQQF |
| | | KSYPLTFGQGTKLEIK |
| SEQ ID NO: 187 | VL | DIQMTQSPSFLSASVGDRVTITCKASQNVDDRV |
| | | AWYQQKPGKAPKALIYSSSHRYKGVPSRFSGSG |
| | | SGTEFTLTISSLQPEDFATYFCQQFKSYPLTFGQG |
| | | TKLEIK |
| SEQ ID NO: 188 | VH | QVQLVQSGAEVKKPGSSVKVSCKASGTDFDKT |
| | | YIHWVRQAPGQGLEWMGRISAGSGNVKYNEKF |
| | | KGRVTITADTSTSTAYMELSSLRSEDTAVYYCA |
| | | GSYYSYDVLDYWGQGTTVTVSS |

A-H.39

| | | |
|---|---|---|
| SEQ ID NO: 189 | VH + VL | QVQLVQSGAEVKKPGSSVKVSCKASGTDFDKIY |
| | | IHWVRQAPGQGLEWMGRISAGSGNIKYNEKFK |
| | | GRVTITADTSTSTAYMELSSLRSEDTAVYYCAGS |
| | | YYSYDVLDYWGQGTTVTVSSGGGGSGGGGSGG |
| | | GGSGGGGSDIQMTQSPSFLSASVGDRVTITCKAS |
| | | QNVDDRVAWYQQKPGKAPKALIYSSSHRYKGV |
| | | PSRFSGSGSGTEFTLTISSLQPEDFATYFCQQFKS |
| | | YPLTFGQGTKLEIK |
| SEQ ID NO: 190 | VL | DIQMTQSPSFLSASVGDRVTITCKASQNVDDRV |
| | | AWYQQKPGKAPKALIYSSSHRYKGVPSRFSGSG |
| | | SGTEFTLTISSLQPEDFATYFCQQFKSYPLTFGQG |
| | | TKLEIK |
| SEQ ID NO: 191 | VH | QVQLVQSGAEVKKPGSSVKVSCKASGTDFDKIY |
| | | IHWVRQAPGQGLEWMGRISAGSGNIKYNEKFK |
| | | GRVTITADTSTSTAYMELSSLRSEDTAVYYCAGS |
| | | YYSYDVLDYWGQGTTVTVSS |

A-H.40

| | | |
|---|---|---|
| SEQ ID NO: 192 | VH + VL | QVQLVQSGAEVKKPGSSVKVSCKASGTDFDKIY |
| | | IHWVRQAPGQGLEWMGRISAGSGNVKYNEKFK |
| | | GRVTITADTSTSTAYMELSSLRSEDTAVYYCAGS |
| | | YYSYDVLDYWGQGTTVTVSSGGGGSGGGGSGG |
| | | GGSGGGGSDIQMTQSPSFLSASVGDRVTITCKAS |
| | | QNVGDRVAWYQQKPGKAPKALIYSSSHRYKGV |
| | | PSRFSGSGSGTEFTLTISSLQPEDFATYFCQQFKS |
| | | YPLTFGQGTKLEIK |
| SEQ ID NO: 193 | VL | DIQMTQSPSFLSASVGDRVTITCKASQNVGDRV |
| | | AWYQQKPGKAPKALIYSSSHRYKGVPSRFSGSG |
| | | SGTEFTLTISSLQPEDFATYFCQQFKSYPLTFGQG |
| | | TKLEIK |
| SEQ ID NO: 194 | VH | QVQLVQSGAEVKKPGSSVKVSCKASGTDFDKIY |
| | | IHWVRQAPGQGLEWMGRISAGSGNVKYNEKFK |
| | | GRVTITADTSTSTAYMELSSLRSEDTAVYYCAGS |
| | | YYSYDVLDYWGQGTTVTVSS |

A-H.41

| | | |
|---|---|---|
| SEQ ID NO: 195 | VH + VL | QVQLVQSGAEVKKPGSSVKVSCKASGGTFKLTY |
| | | IHWVRQAPGQGLEWMGRVSAGSGNVKYNEKF |
| | | KGRVTITADTSTSTAYMELSSLRSEDTAVYYCA |
| | | GSYYSYDVLDYWGQGTTVTVSSGGGGSGGGGS |
| | | GGGGSGGGGSDIQMTQSPSFLSASVGDRVTITCK |
| | | ASQNVDDRVAWYQQKPGKAPKALIYSSSHRYK |
| | | GVPSRFSGSGSGTEFTLTISSLQPEDFATYFCQQF |
| | | KSYPLTFGQGTKLEIK |
| SEQ ID NO: 196 | VL | DIQMTQSPSFLSASVGDRVTITCKASQNVDDRV |
| | | AWYQQKPGKAPKALIYSSSHRYKGVPSRFSGSG |
| | | SGTEFTLTISSLQPEDFATYFCQQFKSYPLTFGQG |
| | | TKLEIK |
| SEQ ID NO: 197 | VH | QVQLVQSGAEVKKPGSSVKVSCKASGGTFKLTY |
| | | IHWVRQAPGQGLEWMGRVSAGSGNVKYNEKF |
| | | KGRVTITADTSTSTAYMELSSLRSEDTAVYYCA |
| | | GSYYSYDVLDYWGQGTTVTVSS |

A-H.42

| | | |
|---|---|---|
| SEQ ID NO: 198 | VH + VL | QVQLVQSGAEVKKPGSSVKVSCKASGTDFKLTY |
| | | IHWVRQAPGQGLEWMGRISPGSGNVKYNEKFK |
| | | GRVTITADTSTSTAYMELSSLRSEDTAVYYCAGS |
| | | YYSYDVLDYWGQGTTVTVSSGGGGSGGGGSGG |
| | | GGSGGGGSDIQMTQSPSFLSASVGDRVTITCKAS |
| | | QNVDNRVAWHQQKPGKAPKALIYSSSHRYKGV |
| | | PSRFSGSGSGTEFTLTISSLQPEDFATYFCQQFKS |
| | | YPLTFGQGTKLEIK |
| SEQ ID NO: 199 | VL | DIQMTQSPSFLSASVGDRVTITCKASQNVDNRV |
| | | AWHQQKPGKAPKALIYSSSHRYKGVPSRFSGSG |
| | | SGTEFTLTISSLQPEDFATYFCQQFKSYPLTFGQG |
| | | TKLEIK |
| SEQ ID NO: 200 | VH | QVQLVQSGAEVKKPGSSVKVSCKASGTDFKLTY |
| | | IHWVRQAPGQGLEWMGRISPGSGNVKYNEKFK |
| | | GRVTITADTSTSTAYMELSSLRSEDTAVYYCAGS |
| | | YYSYDVLDYWGQGTTVTVSS |

A-H.43

| | | |
|---|---|---|
| SEQ ID NO: 201 | VH + VL | QVQLVQSGAEVKKPGSSVKVSCKASGHDFDKF |
| | | YIHWVRQAPGQGLEWMGRVSAGSGNTKYNEKF |
| | | KGRVTITADTSTSTAYMELSSLRSEDTAVYYCA |
| | | GSYYSYDVLDYWGQGTTVTVSSGGGGSGGGGS |
| | | GGGGSGGGGSDIQMTQSPSFLSASVGDRVTITCK |
| | | ASQNVDNRVAWYQQKPGKAPKALIYSSSHRYK |
| | | GVPSRFSGSGSGTEFTLTISSLQPEDFATYFCQQF |
| | | KSYPLTFGQGTKLEIK |
| SEQ ID NO: 202 | VL | DIQMTQSPSFLSASVGDRVTITCKASQNVDNRV |
| | | AWYQQKPGKAPKALIYSSSHRYKGVPSRFSGSG |
| | | SGTEFTLTISSLQPEDFATYFCQQFKSYPLTFGQG |
| | | TKLEIK |
| SEQ ID NO: 203 | VH | QVQLVQSGAEVKKPGSSVKVSCKASGHDFDKF |
| | | YIHWVRQAPGQGLEWMGRVSAGSGNTKYNEKF |
| | | KGRVTITADTSTSTAYMELSSLRSEDTAVYYCA |
| | | GSYYSYDVLDYWGQGTTVTVSS |

A-H.44

| | | |
|---|---|---|
| SEQ ID NO: 204 | VH + VL | QVQLVQSGAEVKKPGSSVKVSCKASGTDFDKFY |
| | | IHWVRQAPGQGLEWMGRVSAGSGNVKYNEKF |
| | | KGRVTITADTSTSTAYMELSSLRSEDTAVYYCA |
| | | GSYYSYDVLDYWGQGTTVTVSSGGGGSGGGGS |
| | | GGGGSGGGGSDIQMTQSPSFLSASVGDRVTITCK |
| | | ASQNVGDRVVWYQQKPGKAPKALIYSSSHRYK |
| | | GVPSRFSGSGSGTEFTLTISSLQPEDFATYFCQQF |
| | | KSYPLTFGQGTKLEIK |
| SEQ ID NO: 205 | VH | QVQLVQSGAEVKKPGSSVKVSCKASGTDFDKFY |
| | | IHWVRQAPGQGLEWMGRVSAGSGNVKYNEKF |
| | | KGRVTITADTSTSTAYMELSSLRSEDTAVYYCA |
| | | GSYYSYDVLDYWGQGTTVTVSS |

A-H.45

| | | |
|---|---|---|
| SEQ ID NO: 206 | VH + VL | QVQLVQSGAEVKKPGSSVKVSCKASGYSFTTYY |
| | | IHWVRQAPGQGLEWMGWFSAGSGNTKYNEKF |
| | | KGRVTITADISTSTAYMELSSLRSEDTAVYYCA |
| | | VSYYSYDVLDYWGQGTTVTVSSGGGGGGGGS |
| | | GGGGSGGGGSDIQMTQSPSFLSASVGDRVTITCK |
| | | ASQNVGINVVWHQQKPGKAPKALIYSSSHRYSG |
| | | VPSRFSGSGSGTEFTLTISSLQPEDFATYFCQQFK |
| | | SYPLTFGQGTKLEIK |
| SEQ ID NO: 207 | VH | QVQLVQSGAEVKKPGSSVKVSCKASGYSFTTYY |
| | | IHWVRQAPGQGLEWMGWFSAGSGNTKYNEKF |
| | | KGRVTITADTSTSTAYMELSSLRSEDTAVYYCA |
| | | VSYYSYDVLDYWGQGTTVTVSS |

A-H.46

| | | |
|---|---|---|
| SEQ ID NO: 208 | VH + VL | QVQLVQSGAEVKKPGSSVKVSCKASGYSFTTYY |
| | | IHWVRQAPGQGLEWMGWFSAGSGNTKYNEKF |
| | | KGRVTITADISTSTAYMELSSLRSEDTAVYYCA |
| | | GSYYSYDVLDYWGQGTTVTVSSGGGGSGGGGS |
| | | GGGGSGGGGSDIQMTQSPSFLSASVGDRVTITCK |
| | | ASQNVGINVVWHQQKPGKAPKALIYSSSHRYSG |
| | | VPSRFSGSGSGTEFTLTISSLQPEDFATYFCQQFK |
| | | SYPLTFGQGTKLEIK |
| SEQ ID NO: 209 | VH | QVQLVQSGAEVKKPGSSVKVSCKASGYSFTTYY |
| | | IHWVRQAPGQGLEWMGWFSAGSGNTKYNEKF |
| | | KGRVTITADTSTSTAYMELSSLRSEDTAVYYCA |
| | | GSYYSYDVLDYWGQGTTVTVSS |

A-H.47

| | | |
|---|---|---|
| SEQ ID NO: 210 | VH + VL | QVQLVQSGABVKKPGSSVKVSCKASGYSFTTYY |
| | | IHWVRQAPGQGLEWMGWFFPGSGNTKYNEKFK |
| | | GRVTITADTSTSTAYMELSSLRSEDTAVYYCAGS |
| | | YYSYDVLDYWGQGTTVTVSSGGGGSGGGGSGG |
| | | GGSGGGGSDIQMTQSPSFLSASVGDRVTITCKAS |
| | | QNVGINVVWHQQKPGKAPKALIYSSSHRYSGVP |
| | | SRFSGSGSGTEFTLTISSLQPEDFATYFCQQFKSY |
| | | PLTFGQGTKLEIK |
| SEQ ID NO: 211 | VH | QVQLVQSGAEVKKPGSSVKVSCKASGYSFTTYY |
| | | IHWVRQAPGQGLEWMGWFFPGSGNTKYNEKFK |
| | | GRVTITADTSTSTAYMELSSLRSEDTAVYYCAGS |
| | | YYSYDVLDYWGQGTTVTVSS |

A-H.48

| | | |
|---|---|---|
| SEQ ID NO: 212 | VH + VL | QVQLVQSGABVKKPGSSVKVSCKASGYSFTTYY |
| | | IHWVRQAPGQGLEWMGWFSPGSGNTKYNEKFK |
| | | GRVTITADTSTSTAYMELSSLRSEDTAVYYCAVS |
| | | YYSYDVLDYWGQGTTVTVSSGGGGSGGGGSGG |
| | | GGSGGGGSDIQMTQSPSFLSASVGDRVTITCKAS |
| | | QNVGINVVWHQQKPGKAPKALIYSSSHRYSGVP |
| | | SRFSGSGSGTEFTLTISSLQPEDFATYFCQQFKSY |
| | | PLTFGQGTKLEIK |
| SEQ ID NO: 213 | VH | QVQLVQSGAEVKKPGSSVKVSCKASGYSFTTYY |
| | | IHWVRQAPGQGLEWMGWFSPGSGNTKYNEKFK |
| | | GRVTITADTSTSTAYMELSSLRSEDTAVYYCAVS |
| | | YYSYDVLDYWGQGTTVTVSS |

A-H.49

| | | |
|---|---|---|
| SEQ ID NO: 214 | VH + VL | QVQLVQSGABVKKPGSSVKVSCKASGYSFTTYY |
| | | IHWVRQAPGQGLEWMGWFSPGSGNTKYNEKFK |
| | | GRVTITADTSTSTAYMELSSLRSEDTAVYYCAGS |
| | | YYSYDVLDYWGQGTTVTVSSGGGGSGGGGSGG |
| | | GGSGGGGSDIQMTQSPSFLSASVGDRVTITCKAS |
| | | QNVGINVVWHQQKPGKAPKALIYSSSHRYSGVP |
| | | SRFSGSGSGTEFTLTISSLQPEDFATYFCQQFKSY |
| | | PLTFGQGTKLEIK |
| SEQ ID NO: 215 | VH | QVQLVQSGAEVKKPGSSVKVSCKASGYSFTTYY |
| | | IHWVRQAPGQGLEWMGWFSPGSGNTKYNEKFK |
| | | GRVTITADTSTSTAYMELSSLRSEDTAVYYCAGS |
| | | YYSYDVLDYWGQGTTVTVSS |

A-H.50

| | | |
|---|---|---|
| SEQ ID NO: 216 | VH + VL | QVQLVQSGAEVKKPGSSVKVSCKASGYSFTTYY |
| | | IHWVRQAPGQGLEWMGRIFPGSGNIKYNEKFKG |
| | | RVTITADTSTSTAYMELSSLRSEDTAVYYCAGSY |
| | | YSYDVLDYWGQGTTVTVSSGGGGSGGGGSGGG |
| | | GSGGGGSDIQMTQSPSFLSASVGDRVTITCKASQ |
| | | NVGINVVWHQQKPGKAPKALTYSSSHRYSGVPS |
| | | RFSGSGSGTEFTLTISSLQPEDFATYFCQQFKSYP |
| | | LTFGQGTKLEIK |
| SEQ ID NO: 217 | VH | QVQLVQSGAEVKKPGSSVKVSCKASGYSFTTYY |
| | | IHWVRQAPGQGLEWMGRIFPGSGNIKYNEKFKG |
| | | RVTITADTSTSTAYMELSSLRSEDTAVYYCAGSY |
| | | YSYDVLDYWGQGTTVTVSS |

A-H.51

| | | |
|---|---|---|
| SEQ ID NO: 218 | VH + VL | QVQLVQSGABVKKPGSSVKVSCKASGYSFTTYY |
| | | IHWVRQAPGQGLEWMGWFFPGSGNIKYNEKFK |
| | | GRVTITADTSTSTAYMELSSLRSEDTAVYYCAGS |
| | | IYSAGVLDYWGQGTTVTVSSGGGGSGGGGSGG |
| | | GGSGGGGSDIQMTQSPSFLSASVGDRVTITCKAS |
| | | QNVGINVVWHQQKPGKAPKALIYSSSHRYSGVP |
| | | SRFSGSGSGTEFTLTISSLQPEDFATYFCQQFKSY |
| | | PLTFGQGTKLEIK |
| SEQ ID NO: 219 | VH | QVQLVQSGAEVKKPGSSVKVSCKASGYSFTTYY |
| | | IHWVRQAPGQGLEWMGWFFPGSGNIKYNEKFK |
| | | GRVTITADTSTSTAYMELSSLRSEDTAVYYCAGS |
| | | IYSAGVLDYWGQGTTVTVSS |

A-H.52

| | | |
|---|---|---|
| SEQ ID NO: 220 | VH + VL | QVQLVQSGAEVKKPGSSVKVSCKASGYSFTLGY |
| | | IHWVRQAPGQGLEWMGWFFPGSGNIKYNEKFK |
| | | GRVTITADTSTSTAYMELSSLRSEDTAVYYCAGS |
| | | YYSYDVLDYWGQGTTVTVSSGGGGSGGGGSGG |
| | | GGSGGGGSDIQMTQSPSFLSASVGDRVTITCKAS |
| | | QNVGINVVWHQQKPGKAPKALIYSSSHRYSGVP |
| | | SRFSGSGSGTEFTLTISSLQPEDFATYFCQQFKSY |
| | | PLTFGQGTKLEIK |
| SEQ ID NO: 221 | VH | QVQLVQSGAEVKKPGSSVKVSCKASGYSFTLGY |
| | | IHWVRQAPGQGLEWMGWFFPGSGNIKYNEKFK |
| | | GRVTITADTSTSTAYMELSSLRSEDTAVYYCAGS |
| | | YYSYDVLDYWGQGTTVTVSS |

A-H.53

| | | |
|---|---|---|
| SEQ ID NO: 222 | VH + VL | QVQLVQSGABVKKPGSSVKVSCKASGYSFRLTY |
| | | IHWVRQAPGQGLEWMGWFFPGSGNIKYNEKFK |
| | | GRVTITADTSTSTAYMELSSLRSEDTAVYYCAGS |
| | | YYSYDVLDYWGQGTTVTVSSGGGGSGGGGSGG |
| | | GGSGGGGSDIQMTQSPSFLSASVGDRVTITCKAS |
| | | QNVGINVVWHQQKPGKAPKALIYSSSHRYSGVP |
| | | SRFSGSGSGTEFTLTISSLQPEDFATYFCQQFKSY |
| | | PLTFGQGTKLEIK |
| SEQ ID NO: 223 | VH | QVQLVQSGAEVKKPGSSVKVSCKASGYSFRLTY |
| | | IHWVRQAPGQGLEWMGWFFPGSGNIKYNEKFK |
| | | GRVTITADTSTSTAYMELSSLRSEDTAVYYCAGS |
| | | YYSYDVLDYWGQGTTVTVSS |

A-H.54

| | | |
|---|---|---|
| SEQ ID NO: 224 | VH + VL | QVQLVQSGAEVKKPGSSVKVSCKASGYSFHNW |
| | | YIHWVRQAPGQGLEWMGWFFPGSGNIKYNEKF |
| | | KGRVTITADTSTSTAYMELSSLRSEDTAVYYCA |
| | | GSYYSYDVLDYWGQGTTVTVSSGGGGSGGGGS |
| | | GGGGSGGGGSDIQMTQSPSFLSASVGDRVTITCK |
| | | ASQNVGINVVWHQQKPGKAPKALIYSSSHRYSG |
| | | VPSRFSGSGSGTEFTLTISSLQPEDFATYFCQQFK |
| | | SYPLTFGQGTKLEIK |
| SEQ ID NO: 225 | VH | QVQLVQSGAEVKKPGSSVKVSCKASGYSFHNW |
| | | YIHWVRQAPGQGLEWMGWFFPGSGNIKYNEKF |
| | | KGRVTITADTSTSTAYMELSSLRSEDTAVYYCA |
| | | GSYYSYDVLDYWGQGTTVTVSS |

A-H.55 antibody

| | | |
|---|---|---|
| SEQ ID NO: 3 | HC CDR1 (Combined) | GYSFTTYYIH |
| SEQ ID NO: 4 | HC CDR2 (Combined) | WFFPGSGNIKYNEKFKG |
| SEQ ID NO: 5 | HC CDR3 (Combined) | SYYSYDVLDY |
| SEQ ID NO: 45 | HC CDR1 (Kabat) | TYYIH |
| SEQ ID NO: 46 | HC CDR2 (Kabat) | WFFPGSGNIKYNEKFKG |
| SEQ ID NO: 47 | HC CDR3 (Kabat) | SYYSYDVLDY |
| SEQ ID NO: 48 | HC CDR1 (Chothia) | GYSFTTY |
| SEQ ID NO: 49 | HC CDR2 (Chothia) | FPGSGN |
| SEQ ID NO: 50 | HC CDR3 (Chothia) | SYYSYDVLDY |
| SEQ ID NO: | VH | QVQLVQSGAEVKKPGSSVKVSCKASGYSFTTYY |
| 1100 | | IHWVRQAPGQGLEWMGWFFPGSGNIKYNEKFK |
| | | GRVTITADTSTSTAYMELSSLRSEDTAVYYCAGS |
| | | YYSYDVLDYWGQGTTVTVSS |
| SEQ ID NO: 6 | LC CDR1 (Combined) | KASQNVGINVV |
| SEQ ID NO: 7 | LC CDR2 (Combined) | SSSHRYS |
| SEQ ID NO: 8 | LC CDR3 (Combined) | QQFKSYPLT |
| SEQ ID NO: 51 | LC CDR1 (Kabat) | KASQNVGINVV |
| SEQ ID NO: 52 | LC CDR2 (Kabat) | SSSHRYS |
| SEQ ID NO: 53 | LC CDR3 (Kabat) | QQFKSYPLT |
| SEQ ID NO: 54 | LC CDR1 (Chothia) | KASQNVGINVV |
| SEQ ID NO: 55 | LC CDR2 (Chothia) | SSSHRYS |
| SEQ ID NO: 56 | LC CDR3 (Chothia) | QQFKSYPLT |
| SEQ ID NO: | VL | QSVLTQPPSVSEAPRQRVTISCKASQNVGINVVW |
| 1101 | | HQQLPGKAPKALIYSSSHRYSGVSDRFSGSGSGT |
| | | SFSLAISGLQSEDEADYFCQQFKSYPLTFGTGTK |
| | | VTVL |

A-H.56

| | | |
|---|---|---|
| SEQ ID NO: | VH + VL (ScFv) | QVQLVQSGAEVKKPGSSVKVSCKASGHDFDKF |
| 1309 | | YIHWVRQAPGQGLEWMGRVSAGSGNVKYNEK |
| | | FKGRVTITADTSTSTAYMELSSLRSEDTAVYYCA |
| | | GSYYSYDVLDYWGQGTTVTVSSGGGGSGGGGS |
| | | GGGGSGGGGSDIQMTQSPSFLSASVGDRVTITCK |
| | | ASQNVGNRVAWYQQKPGKAPKALIYSSSHRYK |
| | | GVPSRFSGSGSGTEFTLTISSLQPEDFATYFCQQF |
| | | KSYPLTFGQGTKLEIK |

A-H.57

| | | |
|---|---|---|
| SEQ ID NO: | VH + VL (ScFv) | QVQLVQSGAEVKKPGSSVKVSCKASGHDFRLTY |
| 1326 | | IHWVRQAPGQGLEWMGRVSAGSGNTKYNEKFK |
| | | GRVTITADTSTSTAYMELSSLRSEDTAVYYCAVS |
| | | YYSYDVLDYWGQGTTVTVSSGGGGSGGGGSGG |
| | | GGSGGGGSDIQMTQSPSFLSASVGDRVTITCKAS |
| | | QNVGDRVVWHQQKPGKAPKALIYSSSHRYKGV |
| | | PSRFSGSGSGTEFTLTISSLQPEDFATYFCQQFKS |
| | | YPLTFGQGTKLEIK |

A-H.58

| | | |
|---|---|---|
| SEQ ID NO: | VH + VL (ScFv) | QVQLVQSGAEVKKPGSSVKVSCKASGHDFRLTY |
| 1327 | | IHWVRQAPGQGLEWMGRVSAGSGNVKYNEKF |
| | | KGRVTITADTSTSTAYMELSSLRSEDTAVYYCA |
| | | VSYYSYDVLDYWGQGTTVTVSSGGGGSGGGGS |
| | | GGGGSGGGGSDIQMTQSPSFLSASVGDRVTITCK |
| | | ASQNVGNRVVWHQQKPGKAPKALIYSSSHRYK |
| | | GVPSRFSGSGSGTEFTLTISSLQPEDFATYFCQQF |
| | | KSYPLTFGQGTKLEIK |

A-H.59

| | | |
|---|---|---|
| SEQ ID NO: | VH + VL (ScFv) | QVQLVQSGAEVKKPGSSVKVSCKASGHDFRLTY |
| 1328 | | IHWVRQAPGQGLEWMGRIYAGSGNVKYNEKFK |
| | | GRVTITADTSTSTAYMELSSLRSEDTAVYYCAVS |
| | | YYSYDVLDYWGQGTTVTVSSGGGGSGGGGSGG |
| | | GGSGGGGSDIQMTQSPSFLSASVGDRVTITCKAS |
| | | QNVADRVVWHQQKPGKAPKALIYSSSHRYKGV |
| | | PSRFSGSGSGTEFTLTISSLQPEDFATYFCQQFKS |
| | | YPLTFGQGTKLEIK |

A-H.60

| | | |
|---|---|---|
| SEQ ID NO: | V H+ VL (ScFv) | QVQLVQSGAEVKKPGSSVKVSCKASGHDFKLT |
| 1329 | | YIHWVRQAPGQGLEWMGRVSAGSGNTKYNEKF |
| | | KGRVTITADTSTSTAYMELSSLRSEDTAVYYCA |
| | | VSYYSYDVLDYWGQGTTVTVSSGGGGSGGGGS |
| | | GGGGSGGGGSDIQMTQSPSFLSASVGDRVTITCK |
| | | ASQNVGDRVAWHQQKPGKAPKALIYSSSHRYK |
| | | GVPSRFSGSGSGTEFTLTISSLQPEDFATYFCQQF |
| | | KSYPLTFGQGTKLEIK |

A-H.61

| | | |
|---|---|---|
| SEQ ID NO: | VH + VL (ScFv) | QVQLVQSGAEVKKPGSSVKVSCKASGHDFKLT |
| 1330 | | YIHWVRQAPGQGLEWMGRVSAGSGNTKYNEKF |
| | | KGRVTITADTSTSTAYMELSSLRSEDTAVYYCA |
| | | VSYYSYDVLDYWGQGTTVTVSSGGGGSGGGGS |
| | | GGGGSGGGGSDIQMTQSPSFLSASVGDRVTITCK |
| | | ASQNVDNRVAWHQQKPGKAPKALIYSSSHRYK |
| | | GVPSRFSGSGSGTEFTLTISSLQPEDFATYFCQQF |
| | | KSYPLTFGQGTKLEIK |

A-H.62

| | | |
|---|---|---|
| SEQ ID NO: | VH + VL (ScFv) | QVQLVQSGAEVKKPGSSVKVSCKASGHDFRLTY |
| 1331 | | IHWVRQAPGQGLEWMGRVSAGSGNVKYNEKF |
| | | KGRVTITADTSTSTAYMELSSLRSEDTAVYYCA |
| | | VSYYSYDVLDYWGQGTTVTVSSGGGGSGGGGS |
| | | GGGGSGGGGSDIQMTQSPSFLSASVGDRVTITCK |
| | | ASQNVADRVVWHQQKPGKAPKALIYSSSHRYK |
| | | GVPSRFSGSGSGTEFTLTISSLQPEDFATYFCQQF |
| | | KSYPLTFGQGTKLEIK |

A-H.63

| | | |
|---|---|---|
| SEQ ID NO: | VH + VL (ScFv) | QVQLVQSGAEVKKPGSSVKVSCKASGHDFRLTY |
| 1332 | | IHWVRQAPGQGLEWMGRVYAGSGNTKYNEKF |
| | | KGRVTITADTSTSTAYMELSSLRSEDTAVYYCA |
| | | VSYYSYDVLDYWGQGTTVTVSSGGGGSGGGGS |
| | | GGGGSGGGGSDIQMTQSPSFLSASVGDRVTITCK |
| | | ASQNVEDRVVWHQQKPGKAPKALIYSSSHRYK |
| | | GVPSRFSGSGSGTEFTLTISSLQPEDFATYFCQQF |
| | | KSYPLTFGQGTKLEIK |

A-H.64

| | | |
|---|---|---|
| SEQ ID NO: | VH + VL (ScFv) | QVQLVQSGAEVKKPGSSVKVSCKASGHDFKLT |
| 1333 | | YIHWVRQAPGQGLEWMGRVSAGSGNTKYNEKF |
| | | KGRVTITADTSTSTAYMELSSLRSEDTAVYYCA |
| | | VSYYSYDVLDYWGQGTTVTVSSGGGGSGGGGS |
| | | GGGGSGGGGSDIQMTQSPSFLSASVGDRVTITCK |
| | | ASQNVADRVVWHQQKPGKAPKALIYSSSHRYK |
| | | GVPSRFSGSGSGTEFTLTISSLQPEDFATYFCQQF |
| | | KSYPLTFGQGTKLEIK |

A-H.65

| | | |
|---|---|---|
| SEQ ID NO: | V H+ VL (ScFv) | QVQLVQSGAEVKKPGSSVKVSCKASGHDFKLT |
| 1334 | | YIHWVRQAPGQGLEWMGRISAGSGNTKYNEKF |
| | | KGRVTITADTSTSTAYMELSSLRSEDTAVYYCA |
| | | VSYYSYDVLDYWGQGTTVTVSSGGGGSGGGGS |
| | | GGGGSGGGGSDIQMTQSPSFLSASVGDRVTITCK |
| | | ASQNVGDRVVWHQQKPGKAPKALIYSSSHRYK |
| | | GVPSRFSGSGSGTEFTLTISSLQPEDFATYFCQQF |
| | | KSYPLTFGQGTKLEIK |

A-H.66

| | | |
|---|---|---|
| SEQ ID NO: | VH + VL (ScFv) | QVQLVQSGAEVKKPGSSVKVSCKASGHDFKLT |
| 1335 | | YIHWVRQAPGQGLEWMGRIYAGSGNTKYNEKF |
| | | KGRVTITADTSTSTAYMELSSLRSEDTAVYYCA |
| | | VSYYSYDVLDYWGQGTTVTVSSGGGGSGGGGS |
| | | GGGGSGGGGSDIQMTQSPSFLSASVGDRVTITCK |
| | | ASQNVGDRVVWHQQKPGKAPKALIYSSSHRYK |
| | | GVPSRFSGSGSGTEFTLTISSLQPEDFATYFCQQF |
| | | KSYPLTFGQGTKLEIK |

A-H.67

| | | |
|---|---|---|
| SEQ ID NO: | VH + VL (ScFv) | QVQLVQSGAEVKKPGSSVKVSCKASGTDFKLTY |
| 1336 | | IHWVRQAPGQGLEWMGRIFPGSGNVKYNEKFK |
| | | GRVTITADTSTSTAYMELSSLRSEDTAVYYCAVS |
| | | YYSYDVLDYWGQGTTVTVSSGGGGSGGGGSGG |
| | | GGSGGGGSDIQMTQSPSFLSASVGDRVTITCKAS |
| | | QNVDNRVAWYQQKPGKAPKALIYSSSHRYKGV |
| | | PSRFSGSGSGTEFTLTISSLQPEDFATYFCQQFKS |
| | | YPLTFGQGTKLEIK |

A-H.68

| | | |
|---|---|---|
| SEQ ID NO: | VH + VL (ScFv) | QVQLVQSGAEVKKPGSSVKVSCKASGHDFRLTY |
| 1337 | | IHWVRQAPGQGLEWMGRISAGSGNVKYNEKFK |
| | | GRVTITADTSTSTAYMELSSLRSEDTAVYYCAVS |
| | | YYSYDVLDYWGQGTTVTVSSGGGGSGGGGSGG |
| | | GGSGGGGSDIQMTQSPSFLSASVGDRVTITCKAS |
| | | QNVADRVAWYQQKPGKAPKALIYSSSHRYKGV |
| | | PSRFSGSGSGTEFTLTISSLQPEDFATYFCQQFKS |
| | | YPLTFGQGTKLEIK |

A-H.69 (also referred to as A-H.13)

| | | |
|---|---|---|
| SEQ ID NO: 110 | VH+ VL (ScFv) | QVQLVQSGAEVKKPGSSVKVSCKASGTDFKLT |
| | | YIHWVRQAPGQGLEWMGRIFPGSGNVKYNEKF |
| | | KGRVTITADTSTSTAYMELSSLRSEDTAVYYCA |
| | | GSYYSYDVLDYWGQGTTVTVSSGGGGGGGG |
| | | SGGGGSGGGGSDIQMTQSPSFLSASVGDRVTI |
| | | TCKASQNVDNRVAWYQQKPGKAPKALIYSSSH |
| | | RYKGVPSRFSGSGSGTEFTLTISSLQPEDFATY |
| | | FCQQFKSYPLTFGQGTKLEIK |

A-H humanized-matured VH

| | | |
|---|---|---|
| SEQ ID NO: | VH-humanized | QVQLVQSGAEVKKPGSSVKVSCKASGTDFKLTY |
| 1310 | matured 1 | IHWVRQAPGQGLEWMGRIFPGSGNVKYNEKFK |
| | | GRVTITADTSTSTAYMELSSLRSEDTAVYYCAGS |
| | | YYSYDVLDYWGQGTTVTVSS |
| SEQ ID NO: | VH-humanized | QVQLVQSGAEVKKPGSSVKVSCKASGTDFKLTY |
| 1311 | matured 2 | IHWVRQAPGQGLEWMGRIFPGSGNVKYNEKFK |
| | | GRVTITADTSTSTAYMELSSLRSEDTAVYYCAVS |
| | | YYSYDVLDYWGQGTTVTVSS |
| SEQ ID NO: | VH-humanized | QVQLVQSGAEVKKPGSSVKVSCKASGHDFRLTY |
| 1312 | matured 3 | IHWVRQAPGQGLEWMGRISAGSGNVKYNEKFK |
| | | GRVTITADTSTSTAYMELSSLRSEDTAVYYCAVS |
| | | YYSYDVLDYWGQGTTVTVSS |

A-H humanized-matured VL

| | | |
|---|---|---|
| SEQ ID NO: | VL-humanized matured | DIQMTQSPSFLSASVGDRVTITCKASQNVDNRV |
| 1313 | 1 | AWYQQKPGKAPKALIYSSSHRYKGVPSRFSGSG |
| | | SGTEFTLTISSLQPEDFATYFCQQFKSYPLTFGQG |
| | | TKLEIK |
| SEQ ID NO: | VL-humanized matured | DIQMTQSPSFLSASVGDRVTITCKASQNVADRV |
| 1314 | 2 | AWYQQKPGKAPKALIYSSSHRYKGVPSRFSGSG |
| | | SGTEFTLTISSLQPEDFATYFCQQFKSYPLTFGQG |
| | | TKLEIK |

A-H.70

| | | |
|---|---|---|
| SEQ ID NO: | VH | QVQLVQSGAEVKKPGSSVKVSCKASGHDFRLTY |
| 1346 | (CDRs underlined) | IHWVRQAPGQGLEWMGRVSAGSGNVKYNEKF |
| | | KGRVTITADTSTSTAYMELSSLRSEDTAVYYCA |
| | | VSYYSYDVLDYWGQGTTVTVSS |
| SEQ ID NO: | VL | DIQMTQSPSFLSASVGDRVTITCKASQNVGNRV |
| 1347 | (CDRs underlined) | VWHQQKPGKAPKALIYSSSHRYKGVPSRFSGSG |
| | | SGTEFTLTISSLQPEDFATYFCQQFKSYPLTFGQG |
| | | TKLEIK |

A-H.71

| | | |
|---|---|---|
| SEQ ID NO: | VH | QVQLVQSGAEVKKPGSSVKVSCKASGHDFRLTY |
| 1348 | (CDRs underlined) | IHWVRQAPGQGLEWMGRIYAGSGNVKYNEKFK |
| | | GRVTITADTSTSTAYMELSSLRSEDTAVYYCAVS |
| | | YYSYDVLDYWGQGTTVTVSS |
| SEQ ID NO: | VL | DIQMTQSPSFLSASVGDRVTITCKASQNVADRV |
| 1349 | (CDRs underlined) | VWHQQKPGKAPKALIYSSSHRYKGVPSRFSGSG |
| | | SGTEFTLTISSLQPEDFATYFCQQFKSYPLTFGQG |
| | | TKLEIK |

A-H.72

| | | |
|---|---|---|
| SEQ ID NO: | VH | QVQLVQSGAEVKKPGSSVKVSCKASGHDFKLT |
| 1350 | (CDRs underlined) | YIHWVRQAPGQGLEWMGRVSAGSGNTKYNEKF |
| | | KGRVTITADTSTSTAYMELSSLRSEDTAVYYCA |
| | | VSYYSYDVLDYWGQGTTVTVSS |
| SEQ ID NO: | VL | DIQMTQSPSFLSASVGDRVTITCKASQNVGDRV |
| 1351 | (CDRs underlined) | AWHQQKPGKAPKALIYSSSHRYKGVPSRFSGSG |
| | | SGTEFTLTISSLQPEDFATYFCQQFKSYPLTFGQG |
| | | TKLEIK |

A-H.73

| | | |
|---|---|---|
| SEQ ID NO: | VH | QVQLVQSGAEVKKPGSSVKVSCKASGHDFKLT |
| 1350 | (CDRs underlined) | YIHWVRQAPGQGLEWMGRVSAGSGNTKYNEKF |
| | | KGRVTITADTSTSTAYMELSSLRSEDTAVYYCA |
| | | VSYYSYDVLDYWGQGTTVTVSS |
| SEQ ID NO: | VL | DIQMTQSPSFLSASVGDRVTITCKASQNVDNRV |
| 1353 | (CDRs underlined) | AWHQQKPGKAPKALIYSSSHRYKGVPSRFSGSG |
| | | SGTEFTLTISSLQPEDFATYFCQQFKSYPLTFGQG |
| | | TKLEIK |

A-H.74

| | | |
|---|---|---|
| SEQ ID NO: | VH | QVQLVQSGAEVKKPGSSVKVSCKASGHDFRLTY |
| 1346 | (CDRs underlined) | IHWVRQAPGQGLEWMGRVSAGSGNVKYNEKF |
| | | KGRVTITADTSTSTAYMELSSLRSEDTAVYYCA |
| | | VSYYSYDVLDYWGQGTTVTVSS |
| SEQ ID NO: | VL | DIQMTQSPSFLSASVGDRVTITCKASQNVADRV |
| 1349 | (CDRs underlined) | VWHQQKPGKAPKALIYSSSHRYKGVPSRFSGSG |
| | | SGTEFTLTISSLQPEDFATYFCQQFKSYPLTFGQG |
| | | TKLEIK |

A-H.75

| | | |
|---|---|---|
| SEQ ID NO: | VH | QVQLVQSGAEVKKPGSSVKVSCKASGHDFRLTY |
| 1356 | (CDRs underlined) | IHWVRQAPGQGLEWMGRVYAGSGNTKYNEKF |
| | | KGRVTITADTSTSTAYMELSSLRSEDTAVYYCA |
| | | VSYYSYDVLDYWGQGTTVTVSS |
| SEQ ID NO: | VL | DIQMTQSPSFLSASVGDRVTITCKASQNVEDRVV |
| 1357 | (CDRs underlined) | WHQQKPGKAPKALIYSSSHRYKGVPSRFSGSGS |
| | | GTEFTLTISSLQPEDFATYFCQQFKSYPLTFGQGT |
| | | KLEIK |

A-H.76

| | | |
|---|---|---|
| SEQ ID NO: | VH | QVQLVQSGAEVKKPGSSVKVSCKASGHDFKLT |
| 1350 | (CDRs underlined) | YIHWVRQAPGQGLEWMGRVSAGSGNTKYNEKF |
| | | KGRVTITADTSTSTAYMELSSLRSEDTAVYYCA |
| | | VSYYSYDVLDYWGQGTTVTVSS |
| SEQ ID NO: | VL | DIQMTQSPSFLSASVGDRVTITCKASQNVADRV |
| 1349 | (CDRs underlined) | VWHQQKPGKAPKALIYSSSHRYKGVPSRFSGSG |
| | | SGTEFTLTISSLQPEDFATYFCQQFKSYPLTFGQG |
| | | TKLEIK |

A-H.77

| | | |
|---|---|---|
| SEQ ID NO: | VH | QVQLVQSGAEVKKPGSSVKVSCKASGHDFKLT |
| 1360 | (CDRs underlined) | YIHWVRQAPGQGLEWMGRISAGSGNTKYNEKF |
| | | KGRVTITADTSTSTAYMELSSLRSEDTAVYYCA |
| | | VSYYSYDVLDYWGQGTTVTVSS |
| SEQ ID NO: | VL | DIQMTQSPSFLSASVGDRVTITCKASQNVGDRV |
| 1361 | (CDRs underlined) | VWHQQKPGKAPKALIYSSSHRYKGVPSRFSGSG |
| | | SGTEFTLTISSLQPEDFATYFCQQFKSYPLTFGQG |
| | | TKLEIK |

A-H.78

| | | |
|---|---|---|
| SEQ ID NO: | VH | QVQLVQSGAEVKKPGSSVKVSCKASGHDFKLT |
| 1362 | (CDRs underlined) | YIHWVRQAPGQGLEWMGRIYAGSGNTKYNEKF |
| | | KGRVTITADTSTSTAYMELSSLRSEDTAVYYCA |
| | | VSYYSYDVLDYWGQGTTVTVSS |
| SEQ ID NO: | VL | DIQMTQSPSFLSASVGDRVTITCKASQNVGDRV |
| 1361 | (CDRs underlined) | VWHQQKPGKAPKALIYSSSHRYKGVPSRFSGSG |
| | | SGTEFTLTISSLQPEDFATYFCQQFKSYPLTFGQG |
| | | TKLEIK |

A-H.79

| | | |
|---|---|---|
| SEQ ID NO: | VH | QVQLVQSGAEVKKPGSSVKVSCKASGHDFKLT |
| 1350 | (CDRs underlined) | YIHWVRQAPGQGLEWMGRVSAGSGNTKYNEKF |
| | | KGRVTITADTSTSTAYMELSSLRSEDTAVYYCA |
| | | VSYYSYDVLDYWGQGTTVTVSS |
| SEQ ID NO: | VL | DIQMTQSPSFLSASVGDRVTITCRASQNVDNRLG |
| 1365 | (CDRs underlined) | WHQQKPGKAPKALIYSSSHRYKGVPSRFSGSGS |
| | | GTEFTLTISSLQPEDFATYFCQQFKSYPLTFGQGT |
| | | KLEIK |

A-H.80

| | | |
|---|---|---|
| SEQ ID NO: | VH | QVQLVQSGAEVKKPGSSVKVSCKASGHDFKLT |
| 1350 | (CDRs underlined) | YIHWVRQAPGQGLEWMGRVSAGSGNTKYNEKF |
| | | KGRVTITADTSTSTAYMELSSLRSEDTAVYYCA |
| | | VSYYSYDVLDYWGQGTTVTVSS |
| SEQ ID NO: | VL | DIQMTQSPSFLSASVGDRVTITCKASQNVDNRV |
| 1367 | (CDRs underlined) | AWHQQKPGKAPKALIYAASSLQKGVPSRFSGSG |
| | | SGTEFTLTISSLQPEDFATYFCQQFKSYPLTFGQG |
| | | TKLEIK |

A-H.81

| | | |
|---|---|---|
| SEQ ID NO: | VH | QVQLVQSGAEVKKPGSSVKVSCKASGHDFKLT |
| 1350 | (CDRs underlined) | YIHWVRQAPGQGLEWMGRVSAGSGNTKYNEKF |
| | | KGRVTITADTSTSTAYMELSSLRSEDTAVYYCA |
| | | VSYYSYDVLDYWGQGTTVTVSS |
| SEQ ID NO: | VL | DIQMTQSPSFLSASVGDRVTITCKASQNVDNRV |
| 1369 | (CDRs underlined) | AWHQQKPGKAPKALIYSSSHRYKGVPSRFSGSG |
| | | SGTEFTLTISSLQPEDFATYFCLQHNSYPLTFGQG |
| | | TKLEIK |

A-H.82

| | | |
|---|---|---|
| SEQ ID NO: | VH | QVQLVQSGAEVKKPGSSVKVSCKASGHDFKLT |
| 1370 | (CDRs underlined) | YIHWVRQAPGQGLEWMGRVSAGSGNVNYAQK |
| | | FQGRVTITADTSTSTAYMELSSLRSEDTAVYYCA |
| | | VSYYSYDVLDYWGQGTTVTVSS |
| SEQ ID NO: | VL | DIQMTQSPSFLSASVGDRVTITCRASQNVDNRLG |
| 1365 | (CDRs underlined) | WHQQKPGKAPKALIYSSSHRYKGVPSRFSGSGS |
| | | GTEFTLTISSLQPEDFATYFCQQFKSYPLTFGQGT |
| | | KLEIK |

A-H.83

| | | |
|---|---|---|
| SEQ ID NO: | VH | QVQLVQSGAEVKKPGSSVKVSCKASGHDFKLT |
| 1370 | (CDRs underlined) | YIHWVRQAPGQGLEWMGRVSAGSGNVNYAQK |
| | | FQGRVTITADTSTSTAYMELSSLRSEDTAVYYCA |
| | | VSYYSYDVLDYWGQGTTVTVSS |
| SEQ ID NO: | VL | DIQMTQSPSFLSASVGDRVTITCKASQNVDNRV |
| 1367 | (CDRs underlined) | AWHQQKPGKAPKALIYAASSLQKGVPSRFSGSG |
| | | SGTEFTLTISSLQPEDFATYFCQQFKSYPLTFGQG |
| | | TKLEIK |

A-H.84

| | | |
|---|---|---|
| SEQ ID NO: | VH | QVQLVQSGAEVKKPGSSVKVSCKASGHDFKLT |
| 1370 | (CDRs underlined) | YIHWVRQAPGQGLEWMGRVSAGSGNVNYAQK |
| | | FQGRVTITADTSTSTAYMELSSLRSEDTAVYYCA |
| | | VSYYSYDVLDYWGQGTTVTVSS |
| SEQ ID NO: | VL | DIQMTQSPSFLSASVGDRVTITCKASQNVDNRV |
| 1369 | (CDRs underlined) | AWHQQKPGKAPKALIYSSSHRYKGVPSRFSGSG |
| | | SGTEFTLTISSLQPEDFATYFCLQHNSYPLTFGQG |
| | | TKLEIK |

A-H.85

| | | |
|---|---|---|
| SEQ ID NO: | VH | QVQLVQSGAEVKKPGSSVKVSCKASGHDFRLTY |
| 1344 | (CDRs underlined) | IHWVRQAPGQGLEWMGRVSAGSGNTKYNEKFK |
| | | GRVTITADTSTSTAYMELSSLRSEDTAVYYCAVS |
| | | YYSYDVLDYWGQGTTVTVSS |
| SEQ ID NO: | VL | DIQMTQSPSFLSASVGDRVTITCKASQNVGDRV |
| 1361 | (CDRs underlined) | VWHQQKPGKAPKALIYSSSHRYKGVPSRFSGSG |
| | | SGTEFTLTISSLQPEDFATYFCQQFKSYPLTFGQG |
| | | TKLEIK |

In some embodiments, the anti-TCRβ3V antibody molecule, e.g., anti-TCRβ3 V6 (e.g., anti-TCRβ V6-5*01) antibody molecule comprises a VH and/or a VL of an antibody described in TABLE 30, or a sequence with at least 80%, 85%, 90%, 95%, 96%, 97%, 98%, 99% or more identity thereto.

In some embodiments, the anti-TCRβV antibody molecule, e.g., anti-TCRβV6 (e.g., anti-TCRβ V6-5*01) antibody molecule comprises a VH and a VL of an antibody described in TABLE 30, or a sequence with at least 80%, 85%, 90%, 95%, 96%, 97%, 98%, 99% or more identity thereto.

In some embodiments, an anti-TCRVb antibody disclosed herein has an antigen binding domain having a VL having a consensus sequence of SEQ ID NO: 230, wherein position 30 is G, E, A or D; position 31 is N or D; position 32 is R or K; position 36 is Y or H; and/or position 56 is K or S.

In some embodiments, an anti-TCRVb antibody disclosed herein has an antigen binding domain having a VH having a consensus sequence of SEQ ID NO: 231, wherein: position 27 is H or T or G or Y; position 28 is D or T or S; position 30 is H or R or D or K or T; position 31 is L or D or K or T or N; position 32 is W or F or T or I or Y or G; position 49 is R or W; position 50 is V or I or F; position 51 is F or S or Y; position 52 is A or P; position 56 is N or S; position 57 is T or V or Y or I; position 58 is K or R; position 97 is G or V; position 99 is Y or I; position 102 is Y or A; and/or position 103 is D or G.

### Anti-TCRβ V12 Antibodies

Accordingly, in one aspect, the disclosure provides an anti-TCRβV antibody molecule that binds to human TCRβ V12, e.g., a TCRβ V12 subfamily comprising: TCRβ V12-4*01, TCRβ V12-3*01 or TCRβ V12-5*01. In some embodiments the TCRβ V12 subfamily comprises TCRβ V12-4*01. In some embodiments the TCRβ V12 subfamily comprises TCRβ V12-3*01.

In some embodiments, the anti-TCRβV antibody molecule, e.g., anti-TCRβ V12 antibody molecule, is a non-murine antibody molecule, e.g., a human or humanized antibody molecule. In some embodiments, the anti-TCRβV antibody molecule, e.g., anti-TCRβ V12 antibody molecule is a human antibody molecule. In some embodiments, the anti-TCRβV antibody molecule, e.g., anti-TCRβ V12 antibody molecule is a humanized antibody molecule.

In some embodiments, the anti-TCRβV antibody molecule, e.g., anti-TCRβ V12 antibody molecule, is isolated or recombinant.

In some embodiments, the anti-TCRβV antibody molecule, e.g., anti-TCRβ V12 antibody molecule, comprises at least one antigen-binding region, e.g., a variable region or an antigen-binding fragment thereof, from an antibody described herein, e.g., an antibody described in Table 31, or encoded by a nucleotide sequence in Table 31, or a sequence substantially identical (e.g., at least 80%, 85%, 90%, 92%, 95%, 97%, 98%, 99% or higher identical) to any of the aforesaid sequences.

In some embodiments, the anti-TCRβV antibody molecule, e.g., anti-TCRβ V12 antibody molecule, comprises at least one, two, three or four variable regions from an antibody described herein, e.g., an antibody as described in Table 31, or encoded by a nucleotide sequence in Table 31, or a sequence substantially identical (e.g., at least 80%, 85%, 90%, 92%, 95%, 97%, 98%, 99% or higher identical) to any of the aforesaid sequences.

In some embodiments, the anti-TCRβV antibody molecule, e.g., anti-TCRβ V12 antibody molecule, comprises at least one or two heavy chain variable regions from an antibody described herein, e.g., an antibody as described in Table 31, or encoded by a nucleotide sequence in Table 31, or a sequence substantially identical (e.g., at least 80%, 85%, 90%, 92%, 95%, 97%, 98%, 99% or higher identical) to any of the aforesaid sequences.

In some embodiments, the anti-TCRβV antibody molecule, e.g., anti-TCRβ V12 antibody molecule, comprises at least one or two light chain variable regions from an antibody described herein, e.g., an antibody as described in Table 31, or encoded by a nucleotide sequence in Table 31, or a sequence substantially identical (e.g., at least 80%, 85%, 90%, 92%, 95%, 97%, 98%, 99% or higher identical) to any of the aforesaid sequences.

In some embodiments, the anti-TCRβV antibody molecule, e.g., anti-TCRβ V12 antibody molecule, comprises a heavy chain constant region for an IgG4, e.g., a human IgG4. In still another embodiment, the anti-TCRβV antibody molecule, e.g., anti-TCRβ V12 antibody molecule, includes a heavy chain constant region for an IgG1, e.g., a human IgG1. In one embodiment, the heavy chain constant region comprises an amino sequence set forth in Table 32, or a sequence substantially identical (e.g., at least 80%, 85%, 90%, 92%, 95%, 97%, 98%, 99% or higher identical) thereto.

In some embodiments, the anti-TCRβV antibody molecule, e.g., anti-TCRβ V12 antibody molecule, includes a kappa light chain constant region, e.g., a human kappa light chain constant region. In one embodiment, the light chain constant region comprises an amino sequence set forth in Table 32, or a sequence substantially identical (e.g., at least 80%, 85%, 90%, 92%, 95%, 97%, 98%, 99% or higher identical) thereto.

In some embodiments, the anti-TCRβV antibody molecule, e.g., anti-TCRβ V12 antibody molecule, includes at least one, two, or three complementarity determining regions (CDRs) from a heavy chain variable region of an antibody described herein, e.g., an antibody as described in Table 31, or encoded by the nucleotide sequence in Table 31, or a sequence substantially identical (e.g., at least 80%, 85%, 90%, 92%, 95%, 97%, 98%, 99% or higher identical) to any of the aforesaid sequences.

In some embodiments, the anti-TCRβV antibody molecule, e.g., anti-TCRβ V12 antibody molecule, includes at least one, two, or three CDRs (or collectively all of the CDRs) from a heavy chain variable region comprising an amino acid sequence shown in Table 31, or encoded by a nucleotide sequence shown in Table 31. In one embodiment, one or more of the CDRs (or collectively all of the CDRs) have one, two, three, four, five, six or more changes, e.g., amino acid substitutions or deletions, relative to the amino acid sequence shown in Table 31, or encoded by a nucleotide sequence shown in Table 31.

In some embodiments, the anti-TCRβV antibody molecule, e.g., anti-TCRβ V12 antibody molecule, includes at least one, two, or three complementarity determining regions (CDRs) from a light chain variable region of an antibody described herein, e.g., an antibody as described in Table 31, or encoded by the nucleotide sequence in Table 31, or a sequence substantially identical (e.g., at least 80%, 85%, 90%, 92%, 95%, 97%, 98%, 99% or higher identical) to any of the aforesaid sequences.

In some embodiments, the anti-TCRβV antibody molecule, e.g., anti-TCRβ V12 antibody molecule, includes at least one, two, or three CDRs (or collectively all of the CDRs) from a light chain variable region comprising an amino acid sequence shown in Table 31, or encoded by a nucleotide sequence shown in Table 31. In one embodiment, one or more of the CDRs (or collectively all of the CDRs) have one, two, three, four, five, six or more changes, e.g., amino acid substitutions or deletions, relative to the amino acid sequence shown in Table 31, or encoded by a nucleotide sequence shown in Table 31.

In some embodiments, the anti-TCRβV antibody molecule, e.g., anti-TCRβ V12 antibody molecule, includes at least one, two, three, four, five or six CDRs (or collectively all of the CDRs) from a heavy and light chain variable region comprising an amino acid sequence shown in Table 31, or encoded by a nucleotide sequence shown in Table 31. In one embodiment, one or more of the CDRs (or collectively all of the CDRs) have one, two, three, four, five, six or more changes, e.g., amino acid substitutions or deletions, relative to the amino acid sequence shown in Table 31, or encoded by a nucleotide sequence shown in Table 31.

In some embodiments, the anti-TCRβV antibody molecule, e.g., anti-TCRβ V12 antibody molecule, molecule includes all six CDRs from an antibody described herein, e.g., an antibody as described in Table 31, or encoded by the nucleotide sequence in Table 31, or closely related CDRs, e.g., CDRs which are identical or which have at least one amino acid alteration, but not more than two, three or four alterations (e.g., substitutions, deletions, or insertions, e.g., conservative substitutions). In some embodiments, the anti-TCRβV antibody molecule, e.g., anti-TCRβ V12 antibody molecule, may include any CDR described herein.

In some embodiments, the anti-TCRβV antibody molecule, e.g., anti-TCRβ V12 antibody molecule includes at least one, two, or three CDRs according to Kabat et al. (e.g., at least one, two, or three CDRs according to the Kabat definition as set out in Table 31) from a heavy chain variable region of an antibody described herein, e.g., an antibody chosen as described in Table 31, or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to one, two, or three CDRs according to Kabat et al. shown in Table 31.

In some embodiments, the anti-TCRβV antibody molecule, e.g., anti-TCRβ V12 antibody molecule includes at least one, two, or three CDRs according to Kabat et al. (e.g., at least one, two, or three CDRs according to the Kabat definition as set out in Table 31) from a light chain variable region of an antibody described herein, e.g., an antibody as described in Table 31, or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to one, two, or three CDRs according to Kabat et al. shown in Table 31.

In some embodiments, the anti-TCRβV antibody molecule, e.g., anti-TCRβ V12 antibody molecule includes at least one, two, three, four, five, or six CDRs according to Kabat et al. (e.g., at least one, two, three, four, five, or six CDRs according to the Kabat definition as set out in Table 31) from the heavy and light chain variable regions of an antibody described herein, e.g., an antibody as described in Table 31, or encoded by the nucleotide sequence in Table 31; or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to one, two, three, four, five, or six CDRs according to Kabat et al. shown in Table 31.

In some embodiments, the anti-TCRβV antibody molecule, e.g., anti-TCRβ V12 antibody molecule includes all six CDRs according to Kabat et al. (e.g., all six CDRs according to the Kabat definition as set out in Table 31) from the heavy and light chain variable regions of an antibody described herein, e.g., an antibody as described in Table 31, or encoded by the nucleotide sequence in Table 31; or encoded by the nucleotide sequence in Table 31; or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to all six CDRs according to Kabat et al. shown in Table 31. In some embodiments, the anti-TCRβV antibody molecule, e.g., anti-TCRβ V12 antibody molecule may include any CDR described herein.

In some embodiments, the anti-TCRβV antibody molecule, e.g., anti-TCRβ V12 antibody molecule includes at least one, two, or three hypervariable loops that have the same canonical structures as the corresponding hypervariable loop of an antibody described herein, e.g., an antibody described in Table 31, e.g., the same canonical structures as at least loop 1 and/or loop 2 of the heavy and/or light chain variable domains of an antibody described herein. See, e.g., Chothia et al., (1992) J. Mol. Biol. 227:799-817; Tomlinson et al., (1992) J. Mol. Biol. 227:776-798 for descriptions of hypervariable loop canonical structures. These structures can be determined by inspection of the tables described in these references.

In some embodiments, the anti-TCRβV antibody molecule, e.g., anti-TCRβ V12 antibody molecule includes at least one, two, or three CDRs according to Chothia et al. (e.g., at least one, two, or three CDRs according to the Chothia definition as set out in Table 31) from a heavy chain variable region of an antibody described herein, e.g., an antibody chosen as described in Table 31, or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to one, two, or three CDRs according to Chothia et al. shown in Table 31.

In some embodiments, the anti-TCRβV antibody molecule, e.g., anti-TCRβ V12 antibody molecule includes at least one, two, or three CDRs according to Chothia et al. (e.g., at least one, two, or three CDRs according to the Chothia definition as set out in Table 31) from a light chain variable region of an antibody described herein, e.g., an antibody as described in Table 31, or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to one, two, or three CDRs according to Chothia et al. shown in Table 31.

In some embodiments, the anti-TCRβV antibody molecule, e.g., anti-TCRβ V12 antibody molecule includes at least one, two, three, four, five, or six CDRs according to Chothia et al. (e.g., at least one, two, three, four, five, or six CDRs according to the Chothia definition as set out in Table 31) from the heavy and light chain variable regions of an antibody described herein, e.g., an antibody as described in Table 31, or encoded by the nucleotide sequence in Table 31; or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to one, two, three, four, five, or six CDRs according to Chothia et al. shown in Table 31.

In some embodiments, the anti-TCRβV antibody molecule, e.g., anti-TCRβ V12 antibody molecule includes all six CDRs according to Chothia et al. (e.g., all six CDRs according to the Chothia definition as set out in Table 31) from the heavy and light chain variable regions of an antibody described herein, e.g., an antibody as described in Table 31, or encoded by the nucleotide sequence in Table 31; or encoded by the nucleotide sequence in Table 31; or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to all six CDRs according to Chothia et al. shown in Table 31. In some embodiments, the anti-TCRβV antibody molecule, e.g., anti-TCRβ V12 antibody molecule may include any CDR described herein.

In some embodiments, the anti-TCRβV antibody molecule, e.g., anti-TCRβ V12 antibody molecule includes at least one, two, or three CDRs according to a combined CDR (e.g., at least one, two, or three CDRs according to the combined CDR definition as set out in Table 31) from a heavy chain variable region of an antibody described herein, e.g., an antibody chosen as described in Table 31, or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to one, two, or three CDRs according to combined CDR shown in Table 31.

In some embodiments, the anti-TCRβV antibody molecule, e.g., anti-TCRβ V12 antibody molecule includes at least one, two, or three CDRs according to a combined CDR (e.g., at least one, two, or three CDRs according to the combined CDR definition as set out in Table 31) from a light chain variable region of an antibody described herein, e.g., an antibody as described in Table 31, or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to one, two, or three CDRs according to a combined CDR shown in Table 31.

In some embodiments, the anti-TCRβV antibody molecule, e.g., anti-TCRβ V12 antibody molecule includes at least one, two, three, four, five, or six CDRs according to a combined CDR. (e.g., at least one, two, three, four, five, or six CDRs according to the combined CDR definition as set out in Table 31) from the heavy and light chain variable regions of an antibody described herein, e.g., an antibody as described in Table 31, or encoded by the nucleotide sequence in Table 31; or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to one, two, three, four, five, or six CDRs according to a combined CDR shown in Table 31.

In some embodiments, the anti-TCRβV antibody molecule, e.g., anti-TCRβ V12 antibody molecule includes all six CDRs according to a combined CDR (e.g., all six CDRs according to the combined CDR definition as set out in Table 31) from the heavy and light chain variable regions of an antibody described herein, e.g., an antibody as described in Table 31, or encoded by the nucleotide sequence in Table 31; or encoded by the nucleotide sequence in Table 31; or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to all six CDRs according to a combined CDR shown in Table 31. In some embodiments, the anti-TCRβV antibody molecule, e.g., anti-TCRβ V12 antibody molecule may include any CDR described herein.

In some embodiments, a combined CDR as set out in TABLE 31 is a CDR that comprises a Kabat CDR and a Chothia CDR.

In some embodiments, the anti-TCRβV antibody molecule, e e.g., anti-TCRβ V12 antibody molecule, molecule includes a combination of CDRs or hypervariable loops identified as combined CDRs in TABLE 31. In some embodiments, the anti-TCRβV antibody molecule, e.g., anti-TCRβ V12 antibody molecule, can contain any combination of CDRs or hypervariable loops according the “combined” CDRs are described in TABLE 31.

In some embodiments, the anti-TCRβV antibody molecule, e.g., anti-TCRβ V12 antibody molecule includes a combination of CDRs or hypervariable loops defined according to the Kabat et al. and Chothia et al., or as described in TABLE 31

In some embodiments, the anti-TCRβV antibody molecule, e.g., anti-TCRβ V12 antibody molecule can contain any combination of CDRs or hypervariable loops according to the Kabat and Chothia definitions.

In an embodiment, e.g., an embodiment comprising a variable region, a CDR (e.g., a combined CDR, Chothia CDR or Kabat CDR), or other sequence referred to herein, e.g., in Table 31, the antibody molecule is a monospecific antibody molecule, a bispecific antibody molecule, a bivalent antibody molecule, a biparatopic antibody molecule, or an antibody molecule that comprises an antigen binding fragment of an antibody, e.g., a half antibody or antigen binding fragment of a half antibody. In some embodiments, the antibody molecule comprises a multispecific molecule, e.g., a bispecific molecule, e.g., as described herein.

In some embodiments, the anti-TCRβV antibody molecule, e.g., anti-TCRβ V12 antibody molecule includes:
(i) one, two or all of a light chain complementarity determining region 1 (LC CDR1), a light chain complementarity determining region 2 (LC CDR2), and a light chain complementarity determining region 3 (LC CDR3) of SEQ ID NO: 16, SEQ ID NO: 26, SEQ ID NO: 27, SEQ ID NO: 28, SEQ ID NO: 29 or SEQ ID NO: 30, and/or
(ii) one, two or all of a heavy chain complementarity determining region 1 (HC CDR1), heavy chain complementarity determining region 2 (HC CDR2), and a heavy chain complementarity determining region 3 (HC CDR3) of SEQ ID NO: 15, SEQ ID NO: 23, SEQ ID NO: 24 or SEQ ID NO: 25.

In some embodiments, the anti-TCRβV antibody molecule, e.g., anti-TCRβ V12 antibody molecule comprises:
(i) a LC CDR1 amino acid sequence of SEQ ID NO: 20, a LC CDR2 amino acid sequence of SEQ ID NO: 21, or a LC CDR3 amino acid sequence of SEQ ID NO: 22; and/or
(ii) a HC CDR1 amino acid sequence of SEQ ID NO: 17, a HC CDR2 amino acid sequence of SEQ ID NO: 18, or a HC CDR3 amino acid sequence of SEQ ID NO: 19.

In some embodiments, the anti-TCRβV antibody molecule, e.g., anti-TCRβ V12 antibody molecule comprises:
(i) a light chain variable region (VL) comprising a LC CDR1 amino acid sequence of SEQ ID NO: 20, a LC CDR2 amino acid sequence of SEQ ID NO: 21, and a LC CDR3 amino acid sequence of SEQ ID NO: 2; and/or
(ii) a heavy chain variable region (VH) comprising a HC CDR1 amino acid sequence of SEQ ID NO: 17, a HC CDR2 amino acid sequence of SEQ ID NO: 18, and a HC CDR3 amino acid sequence of SEQ ID NO: 19.

In some embodiments, the anti-TCRβV antibody molecule, e.g., anti-TCRβ V12 antibody molecule comprises:
(i) a LC CDR1 amino acid sequence of SEQ ID NO: 63, a LC CDR2 amino acid sequence of SEQ ID NO: 64, or a LC CDR3 amino acid sequence of SEQ ID NO: 65; and/or
(ii) a HC CDR1 amino acid sequence of SEQ ID NO: 57, a HC CDR2 amino acid sequence of SEQ ID NO: 58, or a HC CDR3 amino acid sequence of SEQ ID NO: 59.

In some embodiments, the anti-TCRβV antibody molecule, e.g., anti-TCRβ V12 antibody molecule comprises:
(i) a light chain variable region (VL) comprising a LC CDR1 amino acid sequence of SEQ ID NO: 63, a LC CDR2 amino acid sequence of SEQ ID NO: 64, or a LC CDR3 amino acid sequence of SEQ ID NO: 65; and/or
(ii) a heavy chain variable region (VH) comprising a HC CDR1 amino acid sequence of SEQ ID NO: 57, a HC CDR2 amino acid sequence of SEQ ID NO: 58, or a HC CDR3 amino acid sequence of SEQ ID NO: 59.

In some embodiments, the anti-TCRβV antibody molecule, e.g., anti-TCRβ V12 antibody molecule comprises:
(i) a LC CDR1 amino acid sequence of SEQ ID NO: 66, a LC CDR2 amino acid sequence of SEQ ID NO: 67, or a LC CDR3 amino acid sequence of SEQ ID NO: 68; and/or
(ii) a HC CDR1 amino acid sequence of SEQ ID NO: 60, a HC CDR2 amino acid sequence of SEQ ID NO: 61, or a HC CDR3 amino acid sequence of SEQ ID NO: 62.

In some embodiments, the anti-TCRβV antibody molecule, e.g., anti-TCRβ V12 antibody molecule comprises:
(i) a light chain variable region (VL) comprising a LC CDR1 amino acid sequence of SEQ ID NO: 63, a LC CDR2 amino acid sequence of SEQ ID NO: 64, or a LC CDR3 amino acid sequence of SEQ ID NO: 65; and/or
(ii) a heavy chain variable region (VH) comprising a HC CDR1 amino acid sequence of SEQ ID NO: 57, a HC CDR2 amino acid sequence of SEQ ID NO: 58, or a HC CDR3 amino acid sequence of SEQ ID NO: 59.

In one embodiment, the light or the heavy chain variable framework (e.g., the region encompassing at least FR1, FR2, FR3, and optionally FR4) of the anti-TCRβV antibody molecule, e.g., anti-TCRβ V12 antibody molecule can be chosen from: (a) a light or heavy chain variable framework including at least 80%, 85%, 87% 90%, 92%, 93%, 95%, 97%, 98%, or 100% of the amino acid residues from a human light or heavy chain variable framework, e.g., a light or heavy chain variable framework residue from a human mature antibody, a human germline sequence, or a human consensus sequence; (b) a light or heavy chain variable framework including from 20% to 80%, 40% to 60%, 60% to 90%, or 70% to 95% of the amino acid residues from a human light or heavy chain variable framework, e.g., a light or heavy chain variable framework residue from a human mature antibody, a human germline sequence, or a human consensus sequence; (c) a non-human framework (e.g., a rodent framework); or (d) a non-human framework that has been modified, e.g., to remove antigenic or cytotoxic determinants, e.g., deimmunized, or partially humanized. In one embodiment, the light or heavy chain variable framework region (particularly FR1, FR2 and/or FR3) includes a light or heavy chain variable framework sequence at least 70, 75, 80, 85, 87, 88, 90, 92, 94, 95, 96, 97, 98, 99% identical or identical to the frameworks of a VL or VH segment of a human germline gene.

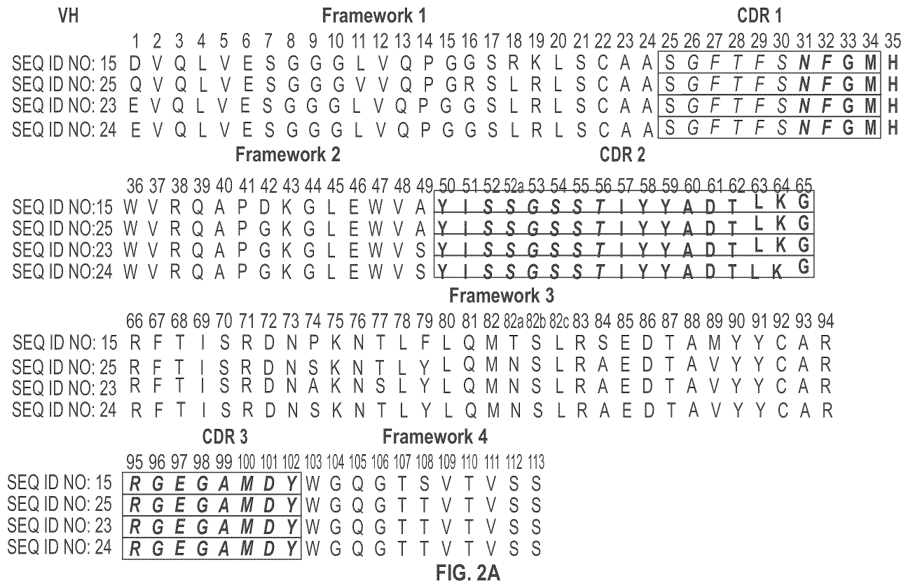

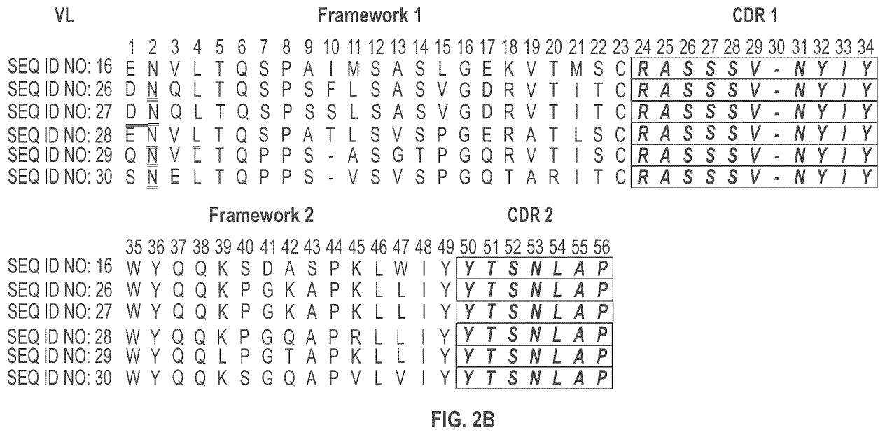

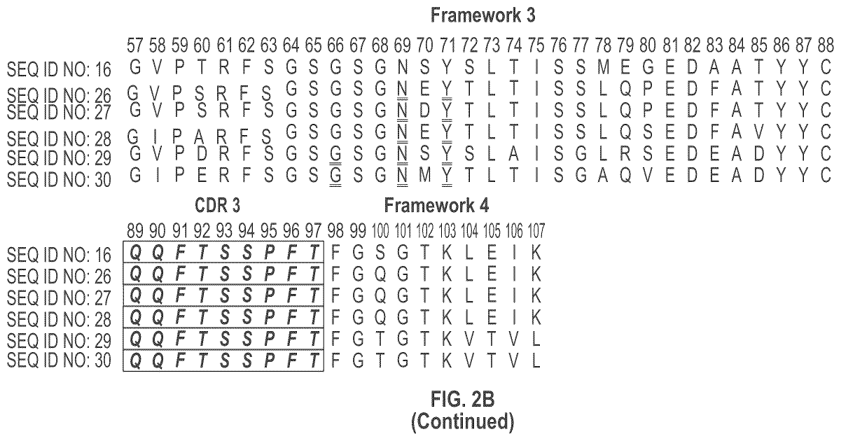

In some embodiments, the anti-TCRβV antibody molecule, e.g., anti-TCRβ V12 antibody molecule, comprises a heavy chain variable domain having at least one, two, three, four, five, six, seven, ten, fifteen, twenty or more changes, e.g., amino acid substitutions or deletions, from an amino acid sequence described in Table 31.e.g., the amino acid sequence of the FR region in the entire variable region, e.g., shown in FIGS. 2A and 2B, or in SEQ ID NOs: 23-25.

Alternatively, or in combination with the heavy chain substitutions described herein the anti-TCRβV antibody molecule, e.g., anti-TCRβ V12 antibody molecule comprises a light chain variable domain having at least one, two, three, four, five, six, seven, ten, fifteen, twenty or more amino acid changes, e.g., amino acid substitutions or deletions, from an amino acid sequence of an antibody described herein e.g., the amino acid sequence of the FR region in the entire variable region, e.g., shown in FIGS. 2A and 2B, or in SEQ ID NOs: 26-30.

In some embodiments, the anti-TCRβV antibody molecule, e.g., anti-TCRβ V12 antibody molecule includes one, two, three, or four heavy chain framework regions shown in FIG. 2A, or a sequence substantially identical thereto.

In some embodiments, the anti-TCRβV antibody molecule, e.g., anti-TCRβ V12 antibody molecule includes one, two, three, or four light chain framework regions shown in FIG. 2B, or a sequence substantially identical thereto.

In some embodiments, the anti-TCRβV antibody molecule, e.g., anti-TCRβ V12 antibody molecule comprises the light chain framework region 1 e.g., as shown in FIG. 2B.

In some embodiments, the anti-TCRβV antibody molecule, e.g., anti-TCRβ V12 antibody molecule comprises the light chain framework region 2 e.g., as shown in FIG. 2B.

In some embodiments, the anti-TCRβV antibody molecule, e.g., anti-TCRβ V12 antibody molecule comprises the light chain framework region 3, e.g., as shown in FIG. 2B.

In some embodiments, the anti-TCRβV antibody molecule, e.g., anti-TCRβ V12 antibody molecule comprises the light chain framework region 4, e.g., as shown in FIG. 2B.

In some embodiments, the anti-TCRβV antibody molecule, e.g., anti-TCRβ V12 antibody molecule comprises a light chain comprising a framework region, e.g., framework region 1 (FR1), comprising a change, e.g., a substitution (e.g., a conservative substitution) at one or more, e.g., all, position disclosed herein according to Kabat numbering. In some embodiments, FR1 comprises an Aspartic Acid at position 1, e.g., a substitution at position 1 according to Kabat numbering, e.g., an Alanine to Aspartic Acid substitution. In some embodiments, FR1 comprises an Asparagine at position 2, e.g., a substitution at position 2 according to Kabat numbering, e.g., an Isoleucine to Asparagine substitution, Serine to Asparagine substitution or Tyrosine to Asparagine substitution. In some embodiments, FR1 comprises a Leucine at position 4, e.g., a substitution at position 4 according to Kabat numbering, e.g., a Methionine to Leucine substitution.

In some embodiments, the anti-TCRβV antibody molecule, e.g., anti-TCRβ V12 antibody molecule comprises a light chain comprising a framework region, e.g., framework region 1 (FR1), comprising a substitution at position 1 according to Kabat numbering, e.g., an Alanine to Aspartic Acid substitution, a substitution at position 2 according to Kabat numbering, e.g., an Isoleucine to Asparagine substitution, Serine to Asparagine substitution or Tyrosine to Asparagine substitution, and a substitution at position 4 according to Kabat numbering, e.g., a Methionine to Leucine substitution. In some embodiments, the anti-TCRβV antibody molecule, e.g., anti-TCRβ V12 antibody molecule comprises a light chain comprising a framework region, e.g., framework region 1 (FR1), comprising a substitution at position 1 according to Kabat numbering, e.g., an Alanine to Aspartic Acid substitution, and a substitution at position 2 according to Kabat numbering, e.g., an Isoleucine to Asparagine substitution, Serine to Asparagine substitution or Tyrosine to Asparagine substitution. In some embodiments, the anti-TCRβV antibody molecule, e.g., anti-TCRβ V12 antibody molecule comprises a light chain comprising a framework region, e.g., framework region 1 (FR1), comprising a substitution at position 1 according to Kabat numbering, e.g., an Alanine to Aspartic Acid substitution, and a substitution at position 4 according to Kabat numbering, e.g., a Methionine to Leucine substitution. In some embodiments, the anti-TCRβV antibody molecule, e.g., anti-TCRβ V12 antibody molecule comprises a light chain comprising a framework region, e.g., framework region 1 (FR1), comprising a substitution at position 2 according to Kabat numbering, e.g., an Isoleucine to Asparagine substitution, Serine to Asparagine substitution or Tyrosine to Asparagine substitution, and a substitution at position 4 according to Kabat numbering, e.g., a Methionine to Leucine substitution. In some embodiments, the substitution is relative to a human germline light chain framework region sequence.

In some embodiments, the anti-TCRβV antibody molecule, e.g., anti-TCRβ V12 antibody molecule comprises a light chain comprising a framework region, e.g., framework region 3 (FR3), comprising a change, e.g., a substitution (e.g., a conservative substitution) at one or more, e.g., all, position disclosed herein according to Kabat numbering. In some embodiments, FR3 comprises a Glycine at position 66, e.g., a substitution at position 66 according to Kabat numbering, e.g., a Lysine to Glycine substitution, or a Serine to Glycine substitution. In some embodiments, FR3 comprises an Asparagine at position 69, e.g., a substitution at position 69 according to Kabat numbering, e.g., a Tyrosine to Asparagine substitution. In some embodiments, FR3 comprises a Tyrosine at position 71, e.g., a substitution at position 71 according to Kabat numbering, e.g., a Phenylalanine to Tyrosine substitution, or an Alanine to Tyrosine substitution.

In some embodiments, the anti-TCRβV antibody molecule, e.g., anti-TCRβ V12 antibody molecule comprises a light chain comprising a framework region, e.g., framework region 3 (FR3), comprising a substitution at position 66 according to Kabat numbering, e.g., a Lysine to Glycine substitution, or a Serine to Glycine substitution, and a substitution at position 69 according to Kabat numbering, e.g., a Tyrosine to Asparagine substitution. In some embodiments, the anti-TCRβV antibody molecule, e.g., anti-TCRβ V12 antibody molecule comprises a light chain comprising a framework region, e.g., framework region 3 (FR3), comprising a substitution at position 66 according to Kabat numbering, e.g., Lysine to Glycine substitution, or a Serine to Glycine substitution, and a substitution at position 71 according to Kabat numbering, e.g., a Phenylalanine to Tyrosine substitution, or an Alanine to Tyrosine substitution. In some embodiments, the anti-TCRβV antibody molecule, e.g., anti-TCRβ V12 antibody molecule comprises a light chain comprising a framework region, e.g., framework region 3 (FR3), comprising a substitution at position 69 according to Kabat numbering, e.g., a Tyrosine to Asparagine substitution and a substitution at position 71 according to Kabat numbering, e.g., a Phenylalanine to Tyrosine substitution, or an Alanine to Tyrosine substitution. In some embodiments, the anti-TCRβV antibody molecule, e.g., anti-TCRβ V12 antibody molecule comprises a light chain comprising a framework region, e.g., framework region 3 (FR3), comprising a substitution at position 66 according to Kabat numbering, e.g., a Lysine to Glycine substitution, or a Serine to Glycine substitution, a substitution at position 69 according to Kabat numbering, e.g., a Tyrosine to Asparagine substitution and a substitution at position 71 according to Kabat numbering, e.g., a Phenylalanine to Tyrosine substitution, or an Alanine to Tyrosine substitution. In some embodiments, the substitution is relative to a human germline light chain framework region sequence.

In some embodiments, the anti-TCRβV antibody molecule, e.g., anti-TCRβ V12 antibody molecule comprises a light chain comprising: a framework region 1 (FR1) comprising a substitution at position 2 according to Kabat numbering, e.g., a Isoleucine to Asparagine substitution; and a framework region 3 (FR3), comprising a substitution at position 69 according to Kabat numbering, e.g., a Threonine to Asparagine substitution and a substitution at position 71 according to Kabat numbering, e.g., a Phenylalanine to Tyrosine substitution, e.g., as shown in the amino acid sequence of SEQ ID NO: 26. In some embodiments, the substitution is relative to a human germline light chain framework region sequence.

In some embodiments, the anti-TCRβV antibody molecule, e.g., anti-TCRβ V12 antibody molecule comprises a light chain comprising: (a) a framework region 1 (FR1) comprising a substitution at position 1 according to Kabat numbering, e.g., a Alanine to Aspartic Acid substitution, and a substitution at position 2 according to Kabat numbering, e.g., a Isoleucine to Asparagine substitution; and (b) a framework region 3 (FR3), comprising a substitution at position 69 according to Kabat numbering, e.g., a Threonine to Asparagine substitution and a substitution at position 71 according to Kabat numbering, e.g., a Phenylalanine to Tyrosine substitution, e.g., as shown in the amino acid sequence of SEQ ID NO: 27 In some embodiments, the substitution is relative to a human germline light chain framework region sequence.

In some embodiments, the anti-TCRβV antibody molecule, e.g., anti-TCRβ V12 antibody molecule comprises a light chain comprising: (a) a framework region 1 (FR1) comprising a substitution at position 2 according to Kabat numbering, e.g., a Serine to Asparagine substitution; and a substitution at position 4 according to Kabat numbering, e.g., a Methionine to Leucine substitution; and (b) a framework region 3 (FR3), comprising a substitution at position 69 according to Kabat numbering, e.g., a Threonine to Asparagine substitution and a substitution at position 71 according to Kabat numbering, e.g., a Phenylalanine to Tyrosine substitution, e.g., as shown in the amino acid sequence of SEQ ID NO: 28 In some embodiments, the substitution is relative to a human germline light chain framework region sequence.

In some embodiments, the anti-TCRβV antibody molecule, e.g., anti-TCRβ V12 antibody molecule comprises a light chain comprising: (a) a framework region 1 (FR1) comprising a substitution at position 2 according to Kabat numbering, e.g., a Serine to Asparagine substitution; and (b) a framework region 3 (FR3) comprising a substitution at position 66 according to Kabat numbering, e.g., a Lysine to Glycine substitution; a substitution at position 69 according to Kabat numbering, e.g., a Threonine to Asparagine substitution; and a substitution at position 71 according to Kabat numbering, e.g., a Alanine to Tyrosine substitution, e.g., as shown in the amino acid sequence of SEQ ID NO: 29. In some embodiments, the substitution is relative to a human germline light chain framework region sequence.

In some embodiments, the anti-TCRβV antibody molecule, e.g., anti-TCRβ V12 antibody molecule comprises a light chain comprising: (a) a framework region 1 (FR1) comprising a substitution at position 2 according to Kabat numbering, e.g., a Tyrosine to Asparagine substitution; and (b) a framework region 3 (FR3) comprising a substitution at position 66 according to Kabat numbering, e.g., a Serine to Glycine substitution; a substitution at position 69 according to Kabat numbering, e.g., a Threonine to Asparagine substitution; and a substitution at position 71 according to Kabat numbering, e.g., a Alanine to Tyrosine substitution, e.g., as shown in the amino acid sequence of SEQ ID NO: 29. In some embodiments, the substitution is relative to a human germline light chain framework region sequence.

In some embodiments, the anti-TCRβV antibody molecule, e.g., anti-TCRβ V12 antibody molecule comprises a light chain variable domain comprising: (a) a framework region 1 (FR1) comprising a change, e.g., a substitution (e.g., a conservative substitution) at one or more (e.g., all) positions disclosed herein according to Kabat numbering, and (b) a framework region 3 (FR3) comprising a change, e.g., a substitution (e.g., a conservative substitution) at one or more (e.g., all) position disclosed herein according to Kabat numbering. In some embodiments, the substitution is relative to a human germline light chain framework region sequence.

In some embodiments, the anti-TCRβV antibody molecule, e.g., anti-TCRβ V12 antibody molecule comprises the heavy chain framework region 1, e.g., as shown in FIG. 2A.

In some embodiments, the anti-TCRβV antibody molecule, e.g., anti-TCRβ V12 antibody molecule comprises the heavy chain framework region 2, e.g., as shown in FIG. 2A.

In some embodiments, the anti-TCRβV antibody molecule, e.g., anti-TCRβ V12 antibody molecule comprises the heavy chain framework region 3, e.g., as shown in FIG. 2A.

In some embodiments, the anti-TCRβV antibody molecule, e.g., anti-TCRβ V12 antibody molecule comprises the heavy chain framework region 4, e.g., as shown in FIG. 2A.

In some embodiments, the anti-TCRβV antibody molecule, e.g., anti-TCRβ V12 antibody molecule comprises the heavy chain framework regions 1-4, e.g., SEQ ID NOS: 20-23, or as shown in FIG. 2A.

In some embodiments, the anti-TCRβV antibody molecule, e.g., anti-TCRβ V12 antibody molecule comprises the light chain framework regions 1-4, e.g., SEQ ID NOs: 26-30, or as shown in FIG. 2B.

In some embodiments, the anti-TCRβV antibody molecule, e.g., anti-TCRβ V12 antibody molecule comprises the heavy chain framework regions 1-4, e.g., SEQ ID NOs: 23-25; and the light chain framework regions 1-4, e.g., SEQ ID NOs: 26-30, or as shown in FIGS. 2A and 2B.

In some embodiments, the heavy or light chain variable domain, or both, of, the anti-TCRβV antibody molecule, e.g., anti-TCRβ V12 antibody molecule includes an amino acid sequence, which is substantially identical to an amino acid disclosed herein, e.g., at least 80%, 85%, 90%, 92%, 95%, 97%, 98%, 99% or higher identical to a variable region of an antibody described herein, e.g., an antibody as described in Table 31, or encoded by the nucleotide sequence in Table 31; or which differs at least 1 or 5 residues, but less than 40, 30, 20, or 10 residues, from a variable region of an antibody described herein.

In some embodiments, the anti-TCRβV antibody molecule, e.g., anti-TCRβ V12 antibody molecule comprises at least one, two, three, or four antigen-binding regions, e.g., variable regions, having an amino acid sequence as set forth in Table 31, or a sequence substantially identical thereto (e.g., a sequence at least about 85%, 90%, 95%, 99% or more identical thereto, or which differs by no more than 1, 2, 5, 10, or 15 amino acid residues from the sequences shown in Table 31. In another embodiment, the anti-TCRβV antibody molecule, e.g., anti-TCRβ V12 antibody molecule includes a VH and/or VL domain encoded by a nucleic acid having a nucleotide sequence as set forth in Table 31, or a sequence substantially identical thereto (e.g., a sequence at least about 85%, 90%, 95%, 99% or more identical thereto, or which differs by no more than 3, 6, 15, 30, or 45 nucleotides from the sequences shown in Table 31.

In some embodiments, the anti-TCRβV antibody molecule, e.g., anti-TCRβ V12 antibody molecule comprises:
a VH domain comprising an amino acid sequence chosen from the amino acid sequence of SEQ ID NO: 23, SEQ ID NO:24 or SEQ ID NO:25, an amino acid sequence at least about 85%, 90%, 95%, 99% or more identical to the amino acid sequence SEQ ID NO: 23, SEQ ID NO:24 or SEQ ID NO:25, or an amino acid sequence which differs by no more than 1, 2, 5, 10, or 15 amino acid residues from the amino acid sequence of SEQ ID NO: 23, SEQ ID NO:24 or SEQ ID NO:25; and/or
a VL domain comprising an amino acid sequence chosen from the amino acid sequence of SEQ ID NO: 26, SEQ ID NO: 27, SEQ ID NO: 28, SEQ ID NO: 29 or SEQ ID NO: 30, an amino acid sequence at least about 85%, 90%, 95%, 99% or more identical to the amino acid sequence of SEQ ID NO: 26, SEQ ID NO: 27, SEQ ID NO: 28, SEQ ID NO: 29 or SEQ ID NO: 30, or an amino acid sequence which differs by no more than 1, 2, 5, 10, or 15 amino acid residues from the amino acid sequence of SEQ ID NO: 26, SEQ ID NO: 27, SEQ ID NO: 28, SEQ ID NO: 29 or SEQ ID NO: 30.

In some embodiments, the anti-TCRβV antibody molecule, e.g., anti-TCRβ V12 antibody molecule comprises:
a VH domain comprising the amino acid sequence of SEQ ID NO: 23, an amino acid sequence at least about 85%, 90%, 95%, 99% or more identical to the amino acid sequence SEQ ID NO: 23, or an amino acid sequence which differs by no more than 1, 2, 5, 10, or 15 amino acid residues from the amino acid sequence of SEQ ID NO: 23; and
a VL domain comprising the amino acid sequence of SEQ ID NO: 26, an amino acid sequence at least about 85%, 90%, 95%, 99% or more identical to the amino acid sequence SEQ ID NO: 26, or an amino acid sequence which differs by no more than 1, 2, 5, 10, or 15 amino acid residues from the amino acid sequence of SEQ ID NO: 26.

In some embodiments, the anti-TCRβV antibody molecule, e.g., anti-TCRβ V12 antibody molecule comprises:
a VH domain comprising the amino acid sequence of SEQ ID NO: 23, an amino acid sequence at least about 85%, 90%, 95%, 99% or more identical to the amino acid sequence SEQ ID NO: 23, or an amino acid sequence which differs by no more than 1, 2, 5, 10, or 15 amino acid residues from the amino acid sequence of SEQ ID NO: 23; and
a VL domain comprising the amino acid sequence of SEQ ID NO: 27, an amino acid sequence at least about 85%, 90%, 95%, 99% or more identical to the amino acid sequence SEQ ID NO: 27, or an amino acid sequence which differs by no more than 1, 2, 5, 10, or 15 amino acid residues from the amino acid sequence of SEQ ID NO: 27.

In some embodiments, the anti-TCRβV antibody molecule, e.g., anti-TCRβ V12 antibody molecule comprises:
a VH domain comprising the amino acid sequence of SEQ ID NO: 23, an amino acid sequence at least about 85%, 90%, 95%, 99% or more identical to the amino acid sequence SEQ ID NO: 23, or an amino acid sequence which differs by no more than 1, 2, 5, 10, or 15 amino acid residues from the amino acid sequence of SEQ ID NO: 23; and
a VL domain comprising the amino acid sequence of SEQ ID NO: 28, an amino acid sequence at least about 85%, 90%, 95%, 99% or more identical to the amino acid sequence SEQ ID NO: 28, or an amino acid sequence which differs by no more than 1, 2, 5, 10, or 15 amino acid residues from the amino acid sequence of SEQ ID NO: 28.

In some embodiments, the anti-TCRβV antibody molecule, e.g., anti-TCRβ V12 antibody molecule comprises:
a VH domain comprising the amino acid sequence of SEQ ID NO: 23, an amino acid sequence at least about 85%, 90%, 95%, 99% or more identical to the amino acid sequence SEQ ID NO: 23, or an amino acid sequence which differs by no more than 1, 2, 5, 10, or 15 amino acid residues from the amino acid sequence of SEQ ID NO: 23; and
a VL domain comprising the amino acid sequence of SEQ ID NO: 29, an amino acid sequence at least about 85%, 90%, 95%, 99% or more identical to the amino acid sequence SEQ ID NO: 29, or an amino acid sequence which differs by no more than 1, 2, 5, 10, or 15 amino acid residues from the amino acid sequence of SEQ ID NO: 29.

In some embodiments, the anti-TCRβV antibody molecule, e.g., anti-TCRβ V12 antibody molecule comprises:
a VH domain comprising the amino acid sequence of SEQ ID NO: 23, an amino acid sequence at least about 85%, 90%, 95%, 99% or more identical to the amino acid sequence SEQ ID NO: 23, or an amino acid sequence which differs by no more than 1, 2, 5, 10, or 15 amino acid residues from the amino acid sequence of SEQ ID NO: 23; and
a VL domain comprising the amino acid sequence of SEQ ID NO: 30, an amino acid sequence at least about 85%, 90%, 95%, 99% or more identical to the amino acid sequence SEQ ID NO: 30, or an amino acid sequence which differs by no more than 1, 2, 5, 10, or 15 amino acid residues from the amino acid sequence of SEQ ID NO: 30.

In some embodiments, the anti-TCRβV antibody molecule, e.g., anti-TCRβ V12 antibody molecule comprises:
a VH domain comprising the amino acid sequence of SEQ ID NO: 24, an amino acid sequence at least about 85%, 90%, 95%, 99% or more identical to the amino acid sequence SEQ ID NO: 24, or an amino acid sequence which differs by no more than 1, 2, 5, 10, or 15 amino acid residues from the amino acid sequence of SEQ ID NO: 24; and
a VL domain comprising the amino acid sequence of SEQ ID NO: 26, an amino acid sequence at least about 85%, 90%, 95%, 99% or more identical to the amino acid sequence SEQ ID NO: 26, or an amino acid sequence which differs by no more than 1, 2, 5, 10, or 15 amino acid residues from the amino acid sequence of SEQ ID NO: 26.

In some embodiments, the anti-TCRβV antibody molecule, e.g., anti-TCRβ V12 antibody molecule comprises:
a VH domain comprising the amino acid sequence of SEQ ID NO: 24, an amino acid sequence at least about 85%, 90%, 95%, 99% or more identical to the amino acid sequence SEQ ID NO: 24, or an amino acid sequence which differs by no more than 1, 2, 5, 10, or 15 amino acid residues from the amino acid sequence of SEQ ID NO: 24; and
a VL domain comprising the amino acid sequence of SEQ ID NO: 27, an amino acid sequence at least about 85%, 90%, 95%, 99% or more identical to the amino acid sequence SEQ ID NO: 27, or an amino acid sequence which differs by no more than 1, 2, 5, 10, or 15 amino acid residues from the amino acid sequence of SEQ ID NO: 27.

In some embodiments, the anti-TCRβV antibody molecule, e.g., anti-TCRβ V12 antibody molecule comprises:
a VH domain comprising the amino acid sequence of SEQ ID NO: 24, an amino acid sequence at least about 85%, 90%, 95%, 99% or more identical to the amino acid sequence SEQ ID NO: 24, or an amino acid sequence which differs by no more than 1, 2, 5, 10, or 15 amino acid residues from the amino acid sequence of SEQ ID NO: 24; and
a VL domain comprising the amino acid sequence of SEQ ID NO: 28, an amino acid sequence at least about 85%, 90%, 95%, 99% or more identical to the amino acid sequence SEQ ID NO: 28, or an amino acid sequence which differs by no more than 1, 2, 5, 10, or 15 amino acid residues from the amino acid sequence of SEQ ID NO: 28.

In some embodiments, the anti-TCRβV antibody molecule, e.g., anti-TCRβ V12 antibody molecule comprises:
a VH domain comprising the amino acid sequence of SEQ ID NO: 24, an amino acid sequence at least about 85%, 90%, 95%, 99% or more identical to the amino acid sequence SEQ ID NO: 24, or an amino acid sequence which differs by no more than 1, 2, 5, 10, or 15 amino acid residues from the amino acid sequence of SEQ ID NO: 24; and
a VL domain comprising the amino acid sequence of SEQ ID NO: 29, an amino acid sequence at least about 85%, 90%, 95%, 99% or more identical to the amino acid sequence SEQ ID NO: 29, or an amino acid sequence which differs by no more than 1, 2, 5, 10, or 15 amino acid residues from the amino acid sequence of SEQ ID NO: 29.

In some embodiments, the anti-TCRβV antibody molecule, e.g., anti-TCRβ V12 antibody molecule comprises:
a VH domain comprising the amino acid sequence of SEQ ID NO: 24, an amino acid sequence at least about 85%, 90%, 95%, 99% or more identical to the amino acid sequence SEQ ID NO: 24, or an amino acid sequence which differs by no more than 1, 2, 5, 10, or 15 amino acid residues from the amino acid sequence of SEQ ID NO: 24; and
a VL domain comprising the amino acid sequence of SEQ ID NO: 30, an amino acid sequence at least about 85%, 90%, 95%, 99% or more identical to the amino acid sequence SEQ ID NO: 30, or an amino acid sequence which differs by no more than 1, 2, 5, 10, or 15 amino acid residues from the amino acid sequence of SEQ ID NO: 30.

In some embodiments, the anti-TCRβV antibody molecule, e.g., anti-TCRβ V12 antibody molecule comprises:
a VH domain comprising the amino acid sequence of SEQ ID NO: 25, an amino acid sequence at least about 85%, 90%, 95%, 99% or more identical to the amino acid sequence SEQ ID NO: 25, or an amino acid sequence which differs by no more than 1, 2, 5, 10, or 15 amino acid residues from the amino acid sequence of SEQ ID NO: 25; and
a VL domain comprising the amino acid sequence of SEQ ID NO: 26, an amino acid sequence at least about 85%, 90%, 95%, 99% or more identical to the amino acid sequence SEQ ID NO: 26, or an amino acid sequence which differs by no more than 1, 2, 5, 10, or 15 amino acid residues from the amino acid sequence of SEQ ID NO: 26.

In some embodiments, the anti-TCRβV antibody molecule, e.g., anti-TCRβ V12 antibody molecule comprises:
a VH domain comprising the amino acid sequence of SEQ ID NO: 25, an amino acid sequence at least about 85%, 90%, 95%, 99% or more identical to the amino acid sequence SEQ ID NO: 25, or an amino acid sequence which differs by no more than 1, 2, 5, 10, or 15 amino acid residues from the amino acid sequence of SEQ ID NO: 25; and
a VL domain comprising the amino acid sequence of SEQ ID NO: 27, an amino acid sequence at least about 85%, 90%, 95%, 99% or more identical to the amino acid sequence SEQ ID NO: 27, or an amino acid sequence which differs by no more than 1, 2, 5, 10, or 15 amino acid residues from the amino acid sequence of SEQ ID NO: 27.

In some embodiments, the anti-TCRβV antibody molecule, e.g., anti-TCRβ V12 antibody molecule comprises:
a VH domain comprising the amino acid sequence of SEQ ID NO: 25, an amino acid sequence at least about 85%, 90%, 95%, 99% or more identical to the amino acid sequence SEQ ID NO: 25, or an amino acid sequence which differs by no more than 1, 2, 5, 10, or 15 amino acid residues from the amino acid sequence of SEQ ID NO: 25; and
a VL domain comprising the amino acid sequence of SEQ ID NO: 28, an amino acid sequence at least about 85%, 90%, 95%, 99% or more identical to the amino acid sequence SEQ ID NO: 28, or an amino acid sequence which differs by no more than 1, 2, 5, 10, or 15 amino acid residues from the amino acid sequence of SEQ ID NO: 28.

In some embodiments, the anti-TCRβV antibody molecule, e.g., anti-TCRβ V12 antibody molecule comprises:
a VH domain comprising the amino acid sequence of SEQ ID NO: 25, an amino acid sequence at least about 85%, 90%, 95%, 99% or more identical to the amino acid sequence SEQ ID NO: 25, or an amino acid sequence which differs by no more than 1, 2, 5, 10, or 15 amino acid residues from the amino acid sequence of SEQ ID NO: 25; and
a VL domain comprising the amino acid sequence of SEQ ID NO: 29, an amino acid sequence at least about 85%, 90%, 95%, 99% or more identical to the amino acid sequence SEQ ID NO: 29, or an amino acid sequence which differs by no more than 1, 2, 5, 10, or 15 amino acid residues from the amino acid sequence of SEQ ID NO: 29.

In some embodiments, the anti-TCRβV antibody molecule, e.g., anti-TCRβ V12 antibody molecule comprises:
a VH domain comprising the amino acid sequence of SEQ ID NO: 25, an amino acid sequence at least about 85%, 90%, 95%, 99% or more identical to the amino acid sequence SEQ ID NO: 25, or an amino acid sequence which differs by no more than 1, 2, 5, 10, or 15 amino acid residues from the amino acid sequence of SEQ ID NO: 25; and
a VL domain comprising the amino acid sequence of SEQ ID NO: 30, an amino acid sequence at least about 85%, 90%, 95%, 99% or more identical to the amino acid sequence SEQ ID NO: 30, or an amino acid sequence which differs by no more than 1, 2, 5, 10, or 15 amino acid residues from the amino acid sequence of SEQ ID NO: 30.

In some embodiments, the anti-TCRβV antibody molecule, e.g., anti-TCRβ V12 antibody molecule is a full antibody or fragment thereof (e.g., a Fab, F(ab′)₂, Fv, or a single chain Fv fragment (scFv)). In some embodiments, the anti-TCRβV antibody molecule, e.g., anti-TCRβ V6 (e.g., anti-TCRβ V6-5*01) antibody molecule is a monoclonal antibody or an antibody with single specificity. In some embodiments, the anti-TCRβV antibody molecule, e.g., anti-TCRβ V12 antibody molecule, can also be a humanized, chimeric, camelid, shark, or an in vitro-generated antibody molecule. In some embodiments, the anti-TCRβV antibody molecule, e.g., anti-TCRβ V12 antibody molecule is a humanized antibody molecule. The heavy and light chains of the anti-TCRβV antibody molecule, e.g., anti-TCRβ V12 antibody molecule can be full-length (e.g., an antibody can include at least one, and preferably two, complete heavy chains, and at least one, and preferably two, complete light chains) or can include an antigen-binding fragment (e.g., a Fab, F(ab′)₂, Fv, a single chain Fv fragment, a single domain antibody, a diabody (dAb), a bivalent antibody, or bispecific antibody or fragment thereof, a single domain variant thereof, or a camelid antibody).

In some embodiments, the anti-TCRβV antibody molecule, e.g., anti-TCRβ V12 antibody molecule is in the form of a multispecific molecule, e.g., a bispecific molecule, e.g., as described herein.

In some embodiments, the anti-TCRβV antibody molecule, e.g., anti-TCRβ V12 antibody molecule has a heavy chain constant region (Fc) chosen from, e.g., the heavy chain constant regions of IgG1, IgG2, IgG3, IgG4, IgM, IgA1, IgA2, IgD, and IgE. In some embodiments, the Fc region is chosen from the heavy chain constant regions of IgG1, IgG2, IgG3, and IgG4. In some embodiments, the Fc region is chosen from the heavy chain constant region of IgG1 or IgG2 (e.g., human IgG1, or IgG2). In some embodiments, the heavy chain constant region is human IgG1.

In some embodiments, the anti-TCRβV antibody molecule, e.g., anti-TCRβ V12 antibody molecule has a light chain constant region chosen from, e.g., the light chain constant regions of kappa or lambda, preferably kappa (e.g., human kappa). In one embodiment, the constant region is altered, e.g., mutated, to modify the properties of the anti-TCRβV antibody molecule, e.g., anti-TCRβ V12 antibody molecule (e.g., to increase or decrease one or more of: Fc receptor binding, antibody glycosylation, the number of cysteine residues, effector cell function, or complement function). For example, the constant region is mutated at positions 296 (M to Y), 298 (S to T), 300 (T to E), 477 (H to K) and 478 (N to F) to alter Fc receptor binding (e.g., the mutated positions correspond to positions 132 (M to Y), 134 (S to T), 136 (T to E), 313 (H to K) and 314 (N to F) of SEQ ID NOs: 212 or 214; or positions 135 (M to Y), 137 (S to T), 139 (T to E), 316 (H to K) and 317 (N to F) of SEQ ID NOs: 215, 216, 217 or 218).

Antibody B-H.1 comprises a first chain comprising the amino acid sequence of SEQ ID NO: 3280 and a second chain comprising the amino acid sequence of SEQ ID NO: 3281.

Additional exemplary anti-TCRβ V12 antibodies of the disclosure are provided in Table 31. In some embodiments, the anti-TCRβ V12 is antibody B, e.g., humanized antibody B (antibody B-H), as provided in Table 31. In some embodiments, the anti-TCRβV antibody comprises one or more (e.g., all three) of a LC CDR1, LC CDR2, and LC CDR3 provided in Table 31; and/or one or more (e.g., all three) of a HC CDR1, HC CDR2, and HC CDR3 provided in Table 31, or a sequence with at least 95% identity thereto. In some embodiments, antibody B comprises a variable heavy chain (VH) and/or a variable light chain (VL) provided in Table 31, or a sequence with at least 95% identity thereto.

TABLE 31
Amino acid and nucleotide sequences for murine and humanized antibody molecules which
bind to TCRVB 12, e.g., TCRVB 12-3 or TCRVB 12-4. The antibody molecules include murine mAb
Antibody B and humanized mAb Antibody B-H.1 to B-H.6. The amino acid the heavy and light chain
CDRs, and the amino acid and nucleotide sequences of the heavy and light chain variable
regions, and the heavy and light chains are shown.
Antibody B (murine), also referred to as 16G8

| | | |
|---|---|---|
| SEQ ID NO: 17 | HC CDR1 (Combined) | GFTFSNFGMH |
| SEQ ID NO: 18 | HC CDR2 (Combined) | YISSGSSTIYYADTLKG |
| SEQ ID NO: 19 | HC CDR3 (Combined) | RGEGAMDY |
| SEQ ID NO: 57 | HC CDR1 (Kabat) | NFGMH |
| SEQ ID NO: 58 | HC CDR2 (Kabat) | YISSGSSTIYYADTLKG |
| SEQ ID NO: 59 | HC CDR3 (Kabat) | RGEGAMDY |
| SEQ ID NO: 60 | HC CDR1 (Chothia) | GFTFSNF |
| SEQ ID NO: 61 | HC CDR2 (Chothia) | SSGSST |
| SEQ ID NO: 62 | HC CDR3 (Chothia) | RGEGAMDY |
| SEQ ID NO: 15 | VH | DVQLVESGGGLVQPGGSRKLSCAASGFTFSNFG |
| | | MHWVRQAPDKGLEWVAYISSGSSTIYYADTLK |
| | | GRFTISRDNPKNTLFLQMTSLRSEDTAMYYCAR |
| | | RGEGAMDYWGQGTSVTVSS |
| SEQ ID NO: 20 | LC CDR1 (Combined) | RASSSVNYIY |
| SEQ ID NO: 21 | LC CDR2 (Combined) | YTSNLAP |
| SEQ ID NO: 22 | LC CDR3(Combined) | QQFTSSPFT |
| SEQ ID NO: 63 | LC CDR1 (Kabat) | RASSSVNYIY |
| SEQ ID NO: 64 | LC CDR2 (Kabat) | YTSNLAP |
| SEQ ID NO: 65 | LC CDR3 (Kabat) | QQFTSSPFT |
| SEQ ID NO: 66 | LC CDR1 (Chothia) | RASSSVNYIY |
| SEQ ID NO: 67 | LC CDR2 (Chothia) | YTSNLAP |
| SEQ ID NO: 68 | LC CDR3 (Chothia) | QQFTSSPFT |
| SEQ ID NO: 16 | VL | ENVLTQSPAIMSASLGEKVTMSCRASSSVNYIY |
| | | WYQQKSDASPKLWIYYTSNLAPGVPTRFSGSGS |
| | | GNSYSLTISSMEGEDAATYYCQQFTSSPFTFGSG |
| | | TKLEIK |

Antibody B humanized (B-H)
Antibody B-H.1A HC-1

| | | |
|---|---|---|
| SEQ ID NO: 17 | HC CDR1 (Combined) | GFTFSNFGMH |
| SEQ ID NO: 18 | HC CDR2 (Combined) | YISSGSSTIYYADTLKG |
| SEQ ID NO: 19 | HC CDR3 (Combined) | RGEGAMDY |
| SEQ ID NO: 23 | VH | EVOLVESGGGLVQPGGSLRLSCAASGFTFSNFG |
| | | MHWVRQAPGKGLEWVSYISSGSSTIYYADTLKG |
| | | RFTISRDNAKNSLYLQMNSLRAEDTAVYYCARR |
| | | GEGAMDYWGQGTTVTVSS |
| SEQ ID NO: 31 | DNA VH | GAGGTGCAGCTGGTTGAATCTGGCGGAGGATT |
| | | GGTTCAGCCTGGCGGCTCTCTGAGACTGTCTT |
| | | GTGCCGCTTCTGGCTTCACCTTCTCCAACTTCG |
| | | GCATGCACTGGGTCCGACAGGCCCCTGGAAAA |
| | | GGACTGGAATGGGTGTCCTACATCTCCTCCGG |
| | | CTCCTCCACCATCTACTACGCTGACACCCTGA |
| | | AGGGCAGATTCACCATCTCTCGGGACAACGCC |
| | | AAGAACTCCCTGTACCTGCAGATGAACAGCCT |
| | | GAGAGCCGAGGACACCGCCGTGTACTACTGTG |
| | | CTAGAAGAGGCGAGGGCGCCATGGATTATTG |
| | | GGGCCAGGGAACCACAGTGACCGTGTCTAGC |

Antibody B-H.1B HC-2

| | | |
|---|---|---|
| SEQ ID NO: 17 | HC CDR1 (Combined) | GFTFSNFGMH |
| SEQ ID NO: 18 | HC CDR2 (Combined) | YISSGSSTIYYADTLKG |
| SEQ ID NO: 19 | HC CDR3 (Combined) | RGEGAMDY |
| SEQ ID NO: 24 | VH | EVQLVESGGGLVQPGGSLRLSCAASGFTFSNFG |
| | | MHWVRQAPGKGLEWVSYISSGSSTIYYADTLKG |
| | | RFTISRDNSKNTLYLQMNSLRAEDTAVYYCARR |
| | | GEGAMDYWGQGTTVTVSS |
| SEQ ID NO: 32 | DNA VH | GAGGTGCAGCTGGTTGAATCTGGCGGAGGATT |
| | | GGTTCAGCCTGGCGGCTCTCTGAGACTGTCTT |
| | | GTGCCGCTTCTGGCTTCACCTTCTCCAACTTCG |
| | | GCATGCACTGGGTCCGACAGGCCCCTGGAAAA |
| | | GGACTGGAATGGGTGTCCTACATCTCCTCCGG |
| | | CTCCTCCACCATCTACTACGCTGACACCCTGA |
| | | AGGGCAGATTCACCATCAGCCGGGACAACTCC |
| | | AAGAACACCCTGTACCTGCAGATGAACTCCCT |
| | | GAGAGCCGAGGACACCGCCGTGTACTACTGTG |
| | | CTAGAAGAGGCGAGGGCGCCATGGATTATTG |
| | | GGGCCAGGGAACCACAGTGACCGTGTCTAGC |

Antibody B-H.1C HC-3

| | | |
|---|---|---|
| SEQ ID NO: 17 | HC CDR1 (Combined) | GFTFSNFGMH |
| SEQ ID NO: 18 | HC CDR2 (Combined) | YISSGSSTIYYADTLKG |
| SEQ ID NO: 19 | HC CDR3 (Combined) | RGEGAMDY |
| SEQ ID NO: 25 | VH | QVQLVESGGGVVQPGRSLRLSCAASGFTFSNFG |
| | | MHWVRQAPGKGLEWVAYISSGSSTIYYADTLK |
| | | GRFTISRDNSKNTLYLQMNSLRAEDTAVYYCAR |
| | | RGEGAMDYWGQGTTVTVSS |
| SEQ ID NO: 33 | DNA VH | CAGGTGCAGCTGGTGGAATCTGGTGGCGGAGT |
| | | TGTGCAGCCTGGCAGATCCCTGAGACTGTCTT |
| | | GTGCCGCCTCTGGCTTCACCTTCTCCAACTTCG |
| | | GCATGCACTGGGTCCGACAGGCCCCTGGAAAA |
| | | GGATTGGAGTGGGTCGCCTACATCTCCTCCGG |
| | | CTCCTCCACCATCTACTACGCTGACACCCTGA |
| | | AGGGCAGATTCACCATCAGCCGGGACAACTCC |
| | | AAGAACACCCTGTACCTGCAGATGAACTCCCT |
| | | GAGAGCCGAGGACACCGCCGTGTACTACTGTG |
| | | CTAGAAGAGGCGAGGGCGCCATGGATTATTG |
| | | GGGCCAGGGAACCACAGTGACCGTGTCTAGC |

Antibody B-H.1D LC-1

| | | |
|---|---|---|
| SEQ ID NO: 20 | LC CDR1 (Combined) | RASSSVNYIY |
| SEQ ID NO: 21 | LC CDR2 (Combined) | YTSNLAP |
| SEQ ID NO: 22 | LC CDR3(Combined) | QQFTSSPFT |
| SEQ ID NO: 26 | VL | DNQLTQSPSFLSASVGDRVTITCRASSSVNYIYW |
| | | YQQKPGKAPKLLIYYTSNLAPGVPSRFSGSGSGN |
| | | EYTLTISSLQPEDFATYYCQQFTSSPFTFGQGTKL |
| | | EIK |
| SEQ ID NO: 34 | DNA VL | GATAACCAGCTGACCCAGTCTCCTAGCTTCCT |
| | | GTCTGCCTCTGTGGGCGACAGAGTGACAATTA |
| | | CCTGCCGGGCCTCCTCCTCCGTGAACTACATCT |
| | | ACTGGTATCAGCAGAAGCCCGGCAAGGCCCCT |
| | | AAGCTGCTGATCTACTACACCTCCAATCTGGC |
| | | CCCTGGCGTGCCCTCTAGATTTTCCGGATCTGG |
| | | CTCCGGCAACGAGTATACCCTGACAATCTCCA |
| | | GCCTGCAGCCTGAGGACTTCGCCACCTACTAC |
| | | TGCCAGCAGTTCACCTCCTCTCCATTCACCTTT |
| | | GGCCAGGGCACCAAGCTGGAAATCAAA |

Antibody B-H.1E LC-2

| | | |
|---|---|---|
| SEQ ID NO: 20 | LC CDR1 (Combined) | RASSSVNYIY |
| SEQ ID NO: 21 | LC CDR2 (Combined) | YTSNLAP |
| SEQ ID NO: 22 | LC CDR3(Combined) | QQFTSSPFT |
| SEQ ID NO: 27 | VL | DNQLTQSPSSLSASVGDRVTITCRASSSVNYIYW |
| | | YQQKPGKAPKLLIYYTSNLAPGVPSRFSGSGSGN |
| | | DYTLTISSLQPEDFATYYCQQFTSSPFTFGQGTKL |
| | | EIK |
| SEQ ID NO: 35 | DNA VL | ATAACCAGCTGACCCAGTCTCCTTCCAGCCTG |
| | | TCTGCTTCTGTGGGCGACAGAGTGACAATTAC |
| | | CTGCCGGGCCTCCTCCTCCGTGAACTACATCT |
| | | ACTGGTATCAGCAGAAGCCCGGCAAGGCCCCT |
| | | AAGCTGCTGATCTACTACACCTCCAATCTGGC |
| | | CCCTGGCGTGCCCTCTAGATTTTCCGGATCTGG |
| | | CTCCGGCAACGACTATACCCTGACAATCTCCA |
| | | GCCTGCAGCCTGAGGACTTCGCCACCTACTAC |
| | | TGCCAGCAGTTCACCTCCTCTCCATTCACCTTT |
| | | GGCCAGGGCACCAAGCTGGAAATCAAA |

Antibody B-H.1F LC-3

| | | |
|---|---|---|
| SEQ ID NO: 20 | LC CDR1 (Combined) | RASSSVNYIY |
| SEQ ID NO: 21 | LC CDR2 (Combined) | YTSNLAP |
| SEQ ID NO: 22 | LC CDR3(Combined) | QQFTSSPFT |
| SEQ ID NO: 28 | VL | ENVLTQSPATLSVSPGERATLSCRASSSVNYIYW |
| | | YQQKPGQAPRLLIYYTSNLAPGIPARFSGSGSGN |
| | | EYTLTISSLQSEDFAVYYCQQFTSSPFTFGQGTKL |
| | | EIK |
| SEQ ID NO: 36 | DNA VL | GAGAATGTGCTGACCCAGTCTCCTGCCACACT |
| | | GTCTGTTAGCCCTGGCGAGAGAGCTACCCTGA |
| | | GCTGCAGAGCCTCTTCCTCCGTGAACTACATC |
| | | TACTGGTATCAGCAGAAGCCCGGCCAGGCTCC |
| | | TAGACTGCTGATCTACTACACCTCCAATCTGG |
| | | CCCCTGGCATCCCTGCCAGATTTTCCGGATCTG |
| | | GCTCCGGCAACGAGTATACCCTGACCATCTCC |
| | | AGCCTGCAGTCCGAGGACTTTGCTGTGTACTA |
| | | TTGCCAGCAGTTCACAAGCAGCCCTTTCACCT |
| | | TTGGCCAGGGCACCAAGCTGGAAATCAAA |

Antibody B-H.1G LC-4

| | | |
|---|---|---|
| SEQ ID NO: 20 | LC CDR1 (Combined) | RASSSVNYIY |
| SEQ ID NO: 21 | LC CDR2 (Combined) | YTSNLAP |
| SEQ ID NO: 22 | LC CDR3(Combined) | QQFTSSPFT |
| SEQ ID NO: 29 | VL | QNVLTQPPSASGTPGQRVTISCRASSSVNYIYWY |
| | | QQLPGTAPKLLIYYTSNLAPGVPDRFSGSGSGNS |
| | | YSLAISGLRSEDEADYYCQQFTSSPFTFGTGTKV |
| | | TVL |
| SEQ ID NO: 37 | DNA VL | CAGAATGTGCTGACCCAACCTCCTTCCGCCTC |
| | | TGGCACACCTGGACAGAGAGTGACAATCTCCT |
| | | GCCGGGCCTCCTCCTCCGTGAACTACATCTAC |
| | | TGGTATCAGCAGCTGCCCGGCACCGCTCCTAA |
| | | ACTGCTGATCTACTACACCTCCAATCTGGCCC |
| | | CTGGCGTGCCCGATAGATTTTCCGGATCTGGC |
| | | TCCGGCAACTCCTACAGCCTGGCTATCTCTGG |
| | | CCTGAGATCTGAGGACGAGGCCGACTACTACT |
| | | GCCAGCAGTTCACCTCCTCTCCATTCACCTTTG |
| | | GCACCGGCACCAAAGTGACAGTTCTT |

Antibody B-H.1H LC-5

| | | |
|---|---|---|
| SEQ ID NO: 20 | LC CDR1 (Combined) | RASSSVNYIY |
| SEQ ID NO: 21 | LC CDR2 (Combined) | YTSNLAP |
| SEQ ID NO: 22 | LC CDR3 (Combined) | QQFTSSPFT |
| SEQ ID NO: 30 | VL | SNELTQPPSVSVSPGQTARITCRASSSVNYIYWY |
| | | QQKSGQAPVLVIYYTSNLAPGIPERFSGSGSGNM |
| | | YTLTISGAQVEDEADYYCQQFTSSPFTFGTGTKV |
| | | TVL |
| SEQ ID NO: 38 | DNA VL | TCTAATGAGCTGACCCAGCCTCCTTCCGTGTCC |
| | | GTGTCTCCTGGACAGACCGCCAGAATTACCTG |
| | | CCGGGCCTCCTCCTCCGTGAACTACATCTACT |
| | | GGTATCAGCAGAAGTCCGGCCAGGCTCCTGTG |
| | | CTCGTGATCTACTACACCTCCAATCTGGCCCCT |
| | | GGCATCCCTGAGAGATTCTCCGGATCTGGCTC |
| | | CGGCAACATGTACACCCTGACCATCTCTGGCG |
| | | CCCAGGTGGAAGATGAGGCCGACTACTACTGC |
| | | CAGCAGTTCACCTCCTCTCCATTCACCTTTGGC |
| | | ACCGGCACCAAAGTGACAGTTCTT |

Antibody B-H.1

| | | |
|---|---|---|
| SEQ ID NO: | Chain1: Fc only | METDTLLLWVLLLWVPGSTGDKTHTCPPCPAPE |
| 3280 | | LLGGPSVFLFPPKPKDTLMISRTPEVTCVVVDVS |
| | | HEDPEVKFNWYVDGVEVHNAKTKPREEQYNST |
| | | YRVVSVLTVLHQDWLNGKEYKCKVSNKALPAP |
| | | IEKTISKAKGQPREPQVYTLPPCREEMTKNQVSL |
| | | WCLVKGFYPSDIAVEWESNGQPENNYKTTPPVL |
| | | DSDGSFFLYSKLTVDKSRWQQGNVFSCSVMHE |
| | | ALHNRFTQKSLSLSPGK |
| SEQ ID NO: | Chain2: humanized B-H | METDTLLLWVLLLWVPGSTGEVQLVESGGGLV |
| 3281 | scFv | QPGGSLRLSCAASGFTFSNFGMHWVRQAPGKGL |
| | | EWVSYISSGSSTIYYADTLKGRFTISRDNSKNTLY |
| | | LQMNSLRAEDTAVYYCARRGEGAMDYWGQGT |
| | | TVTVSSGGGGSGGGGSGGGGSGGGGSDNQLTQ |
| | | SPSFLSASVGDRVTITCRASSSVNYIYWYQQKPG |
| | | KAPKLLIYYTSNLAPGVPSRFSGSGSGNEYTLTIS |
| | | SLQPEDFATYYCQQFTSSPFTFGQGTKLEIKGGG |
| | | GSDKTHTCPPCPAPELLGGPSVFLFPPKPKDTLMI |
| | | SRTPEVTCVVVDVSHEDPEVKFNWYVDGVEVH |
| | | NAKTKPREEQYNSTYRVVSVLTVLHQDWLNGK |
| | | EYKCKVSNKALPAPIEKTISKAKGQPREPQVCTL |
| | | PPSREEMTKNQVSLSCAVKGFYPSDIAVEWESN |
| | | GQPENNYKTTPPVLDSDGSFFLVSKLTVDKSRW |
| | | QQGNVFSCSVMHEALHNHYTQKSLSLSPGKGG |
| | | GGSGGGGSGLNDIFEAQKIEWHE |
| SEQ ID NO: | scFv | EVQLVESGGGLVQPGGSLRLSCAASGFTFSNFG |
| 7492 | | MHWVRQAPGKGLEWVSYISSGSSTIYYADTLKG |
| | | RFTISRDNSKNTLYLQMNSLRAEDTAVYYCARR |
| | | GEGAMDYWGQGTTVTVSSGGGGSGGGGSGGG |
| | | GSGGGGSDNQLTQSPSFLSASVGDRVTITCRASS |
| | | SVNYIYWYQQKPGKAPKLLIYYTSNLAPGVPSR |
| | | FSGSGSGNEYTLTISSLQPEDFATYYCQQFTSSPF |
| | | TFGQGTKLEIK |

Antibody B-H.2

| | | |
|---|---|---|
| SEQ ID NO: | scFv | EVQLVESGGGLVQPGGSLRLSCAASGFTFSNFG |
| 1338 | | MHWVRQAPGKGLEWVSYISSGSSTIYYADTLKG |
| | | RFTISRDNSKNTLYLQMNSLRAEDTAVYYCARR |
| | | GEGAMDYWGQGTTVTVSSGGGGSGGGGSGGG |
| | | GSGGGGSDNQLTQSPSSLSASVGDRVTITCRASS |
| | | SVNYIYWYQQKPGKAPKLLIYYTSNLAPGVPSR |
| | | FSGSGSGNDYTLTISSLQPEDFATYYCQQFTSSPF |
| | | TFGQGTKLEIK |

Antibody B-H.3

| | | |
|---|---|---|
| SEQ ID NO: | scFv | EVQLVESGGGLVQPGGSLRLSCAASGFTFSNFG |
| 1339 | | MHWVRQAPGKGLEWVSYISSGSSTIYYADTLKG |
| | | RFTISRDNSKNTLYLQMNSLRAEDTAVYYCARR |
| | | GEGAMDYWGQGTTVTVSSGGGGSGGGGSGGG |
| | | GSGGGGSSNELTQPPSVSVSPGQTARITCRASSS |
| | | VNYIYWYQQKSGQAPVLVIYYTSNLAPGIPERFS |
| | | GSGSGNMYTLTISGAQVEDEADYYCQQFTSSPF |
| | | TFGTGTKVTVL |

Antibody B-H.4

| | | |
|---|---|---|
| SEQ ID NO: | scFv | QVQLVESGGGVVQPGRSLRLSCAASGFTFSNFG |
| 1340 | | MHWVRQAPGKGLEWVAYISSGSSTIYYADTLK |
| | | GRFTISRDNSKNTLYLQMNSLRAEDTAVYYCAR |
| | | RGEGAMDYWGQGTTVTVSSGGGGSGGGGSGG |
| | | GGSGGGGSDNQLTQSPSFLSASVGDRVTITCRAS |
| | | SSVNYIYWYQQKPGKAPKLLIYYTSNLAPGVPS |
| | | RFSGSGSGNEYTLTISSLQPEDFATYYCQQFTSSP |
| | | FTFGQGTKLEIK |

Antibody B-H.5

| | | |
|---|---|---|
| SEQ ID NO: | scFv | QVQLVESGGGVVQPGRSLRLSCAASGFTFSNFG |
| 1341 | | MHWVRQAPGKGLEWVAYISSGSSTIYYADTLK |
| | | GRFTISRDNSKNTLYLQMNSLRAEDTAVYYCAR |
| | | RGEGAMDYWGQGTTVTVSSGGGGSGGGGSGG |
| | | GGSGGGGSDNQLTQSPSSLSASVGDRVTITCRAS |
| | | SSVNYIYWYQQKPGKAPKLLIYYTSNLAPGVPS |
| | | RFSGSGSGNDYTLTISSLQPEDFATYYCQQFTSSP |
| | | FTFGQGTKLEIK |

Antibody B-H.6

| | | |
|---|---|---|
| SEQ ID NO: | scFv | QVQLVESGGGVVQPGRSLRLSCAASGFTFSNFG |
| 1342 | | MHWVRQAPGKGLEWVAYISSGSSTIYYADTLK |
| | | GRFTISRDNSKNTLYLQMNSLRAEDTAVYYCAR |
| | | RGEGAMDYWGQGTTVTVSSGGGGSGGGGSGG |
| | | GGSGGGGSSNELTQPPSVSVSPGQTARITCRASS |
| | | SVNYIYWYQQKSGQAPVLVIYYTSNLAPGIPERF |
| | | SGSGSGNMYTLTISGAQVEDEADYYCQQFTSSPF |
| | | TFGTGTKVTVL |

TABLE 32
Constant region amino acid sequences of human IgG heavy chains and human kappa
light chain

| | | |
|---|---|---|
| Human kappa | LC | RTVAAPSVFI FPPSDEQLKS GTASVVCLLN NFYPREAKVQ |
| constant region | | WKVDNALQSG NSQESVTEQD SKDSTYSLSS TLTLSKADYE |
| SEQ ID NO: 39 | | KHKVYACEVT HQGLSSPVTK SFNRGEC |
| IgG4 (S228P) | HC | ASTKGPSVFPLAPCSRSTSESTAALGCLVKDYFPEPVTVSWNSGA |
| mutant constant | | LTSGVHTFPAVLQSSGLYSLSSVVTVPSSSLGTKTYTCNVDHKPS |
| region (EU | | NTKVDKRVESKYGPPCPPCPAPEFLGGPSVFLFPPKPKDTLMISRT |
| Numbering) | | PEVTCVVVDVSQEDPEVQFNWYVDGVEVHNAKTKPREEQFNST |
| SEQ ID NO: 40 | | YRVVSVLTVLHQDWLNGKEYKCKVSNKGLPSSIEKTISKAKGQP |
| | | REPQVYTLPPSQEEMTKNQVSLTCLVKGFYPSDIAVEWESNGQPE |
| | | NNYKTTPPVLDSDGSFFLYSRLTVDKSRWQEGNVFSCSVMHEAL |
| | | HNHYTQKSLSLSLG |
| IgG1 wild type | HC | ASTKGPSVFPLAPSSKSTSGGTAALGCLVKDYFPEPVTVSWNSGA |
| SEQ ID NO: 41 | | LTSGVHTFPAVLQSSGLYSLSSVVTVPSSSLGTQTYICNVNHKPSN |
| | | TKVDKRVEPKSCDKTHTCPPCPAPELLGGPSVFLFPPKPKDTLMIS |
| | | RTPEVTCVVVDVSHEDPEVKFNWYVDGVEVHNAKTKPREEQYN |
| | | STYRVVSVLTVLHQDWLNGKEYKCKVSNKALPAPIEKTISKAKG |
| | | QPREPQVYTLPPSREEMTKNQVSLTCLVKGFYPSDIAVEWESNGQ |
| | | PENNYKTTPPVLDSDGSFFLYSKLTVDKSRWQQGNVFSCSVMHE |
| | | ALHNHYTQKSLSLSPGK |
| IgG1 (N297A) | HC | ASTKGPSVFPLAPSSKSTSGGTAALGCLVKDYFPEPVTVSWNSGA |
| mutant constant | | LTSGVHTFPAVLQSSGLYSLSSVVTVPSSSLGTQTYICNVNHKPSN |
| region (EU | | TKVDKRVEPKSCDKTHTCPPCPAPELLGGPSVFLFPPKPKDTLMIS |
| Numbering) | | RTPEVTCVVVDVSHEDPEVKFNWYVDGVEVHNAKTKPREEQYA |
| SEQ ID NO: 42 | | STYRVVSVLTVLHQDWLNGKEYKCKVSNKALPAPIEKTISKAKG |
| | | QPREPQVYTLPPSREEMTKNQVSLTCLVKGFYPSDIAVEWESNGQ |
| | | PENNYKTTPPVLDSDGSFFLYSKLTVDKSRWQQGNVFSCSVMHE |
| | | ALHNHYTQKSLSLSPGK |
| IgM constant | HC | GSASAPTLFPLVSCENSPSDTSSVAVGCLAQDFLPDSITFSWKYKN |
| delta CDC | | NSDISSTRGFPSVLRGGKYAATSQVLLPSKDVMQGTDEHVVCKV |
| (P311A, P313S) | | QHPNGNKEKNVPLPVIAELPPKVSVFVPPRDGFFGNPRKSKLICQ |
| SEQ ID NO: 73 | | ATGFSPRQIQVSWLREGKQVGSGVTTDQVQAEAKESGPTTYKVT |
| | | STLTIKESDWLGQSMFTCRVDHRGLTFQQNASSMCVPDQDTAIR |
| | | VFAIPPSFASIFLTKSTKLTCLVTDLTTYDSVTISWTRQNGEAVKT |
| | | HTNISESHPNATFSAVGEASICEDDWNSGERFTCTVTHTDLASSL |
| | | KQTISRPKGVALHRPDVYLLPPAREQLNLRESATITCLVTGFSPAD |
| | | VFVQWMQRGQPLSPEKYVTSAPMPEPQAPGRYFAHSILTVSEEE |
| | | WNTGETYTCVVAHEALPNRVTERTVDKSTGKPTLYNVSLVMSD |
| | | TAGTCY |
| IgGA1 | HC | ASPTSPKVFPLSLCSTQPDGNVVIACLVQGFFPQEPLSVTWSESGQ |
| SEQ ID NO: 74 | | GVTARNFPPSQDASGDLYTTSSQLTLPATQCLAGKSVTCHVKHY |
| | | TNPSQDVTVPCPVPSTPPTPSPSTPPTPSPSCCHPRLSLHRPALEDL |
| | | LLGSEANLTCTLTGLRDASGVTFTWTPSSGKSAVQGPPERDLCGC |
| | | YSVSSVLPGCAEPWNHGKTFTCTAAYPESKTPLTATLSKSGNTFR |
| | | PEVHLLPPPSEELALNELVTLTCLARGFSPKDVLVRWLQGSQELP |
| | | REKYLTWASRQEPSQGTTTFAVTSILRVAAEDWKKGDTFSCMVG |
| | | HEALPLAFTQKTIDRLAGKPTHVNVSVVMAEVDGTCY |
| IgGA2 | HC | ASPTSPKVFPLSLDSTPQDGNVVVACLVQGFFPQEPLSVTWSESG |
| SEQ ID NO: 75 | | QNVTARNFPPSQDASGDLYTTSSQLTLPATQCPDGKSVTCHVKH |
| | | YTNSSQDVTVPCRVPPPPPCCHPRLSLHRPALEDLLLGSEANLTCT |
| | | LTGLRDASGATFTWTPSSGKSAVQGPPERDLCGCYSVSSVLPGCA |
| | | QPWNHGETFTCTAAHPELKTPLTANITKSGNTFRPEVHLLPPPSEE |
| | | LALNELVTLTCLARGFSPKDVLVRWLQGSQELPREKYLTWASRQ |
| | | EPSQGTTTYAVTSILRVAAEDWKKGETFSCMVGHEALPLAFTQK |
| | | TIDRMAGKPTHINVSVVMAEADGTCY |
| Human Ig_J | HC | MKNHLLFWGVLAVFIKAVHVKAQEDERIVLVDNKCKCARITSRII |
| chain | | RSSEDPNEDIVERNIRIIVPLNNRENISDPTSPLRTRFVYHLSDLCK |
| SEQ ID NO: 76 | | KCDPTEVELDNQIVTATQSNICDEDSATETCYTYDRNKCYTAVVP |
| | | LVYGGETKMVETALTPDACYPD |

### Anti-TCRβ V5 Antibodies

Accordingly, in one aspect, the disclosure provides an anti-TCRβV antibody molecule that binds to human TCRβ V5. In some embodiments, the TCRβ V5 subfamily comprises TCRβ V5-5*01, TCRβ V5-6*01, TCRβ V5-4*01, TCRβ V5-8*01, TCRβ V5-1*01, or a variant thereof.

Exemplary anti-TCRβ V5 antibodies of the disclosure are provided in Table 33. In some embodiments, the anti-TCRβ3 VS is antibody C, e.g., humanized antibody C (antibody C-H), as provided in Table 33. In some embodiments, the anti-TCRβV antibody comprises one or more (e.g., all three) of a LC CDR, LC CDR2, and LC CDR3 provided in Table 33; and/or one or more (e.g., all three) of a HC CDR1, HC CDR2, and HC CDR3 provided in Table 33, or a sequence with at least 95% identity thereto. In some embodiments, antibody C comprises a variable heavy chain (VH) and/or a variable light chain (VL) provided in Table 33, or a sequence with at least 95% identity thereto.

TABLE 33
Amino acid sequences for anti TCRβ V5 antibodies
Amino acid and nucleotide sequences for murine and humanized antibody molecules which bind to
TCRVB 5 (e.g., TCRVB 5-5 or TCRVB 5-6). The amino acid the heavy and light chain CDRs, and the
amino acid and nucleotide sequences of the heavy and light chain variable regions, and the
heavy and light chains are shown.
Murine antibody C, also referred to as 4H11

| | | |
|---|---|---|
| SEQ ID NO: 1315 | HC CDR1 (Kabat) | AYGVN |
| SEQ ID NO: 1316 | HC CDR2 (Kabat) | MIWGDGNTDYNSALKS |
| SEQ ID NO: 1317 | HC CDR3 (Kabat) | DRVTATLYAMDY |
| SEQ ID NO: 1318 | HC CDR1 (Chothia) | GFSLTAY |
| SEQ ID NO: 1319 | HC CDR2 (Chothia) | WGDGN |
| SEQ ID NO: 1317 | HC CDR3 (Chothia) | DRVTATLYAMDY |
| SEQ ID NO: 1320 | HC CDR1 (Combined) | GFSLTAYGVN |
| SEQ ID NO: 1316 | HC CDR2 (Combined) | MIWGDGNTDYNSALKS |
| SEQ ID NO: 1317 | HC CDR3(Combined) | DRVTATLYAMDY |
| SEQ ID NO: 1321 | LC CDR1 (Kabat) | SASQGISNYLN |
| SEQ ID NO: 1322 | LC CDR2 (Kabat) | YTSSLHS |
| SEQ ID NO: 1323 | LC CDR3 (Kabat) | QQYSKLPRT |
| SEQ ID NO: 1321 | LC CDR1 (Chothia) | SASQGISNYLN |
| SEQ ID NO: 1322 | LC CDR2 (Chothia) | YTSSLHS |
| SEQ ID NO: 1323 | LC CDR3 (Chothia) | QQYSKLPRT |
| SEQ ID NO: 1321 | LC CDR1 (Combined) | SASQGISNYLN |
| SEQ ID NO: 1322 | LC CDR2 (Combined) | YTSSLHS |
| SEQ ID NO: 1323 | LC CDR3(Combined) | QQYSKLPRT |
| SEQ ID NO: 232 | VH | DIQMTQTTSSLSASLGDRVTISCSASQGISNY |
| | | LNWYQQKPDGTVKLLIYYTSSLHSGVPSRFS |
| | | GSGSGTDYSLTISNLEPEDIATYYCQQYSKLP |
| | | RTFGGGTKVEIK |
| SEQ ID NO: 7488 | VL | QVQLKESGPGLVAPSQSLSITCTVSGFSLTAY |
| | | GVNWVRQPPGKGLEWLGMIWGDGNTDYN |
| | | SALKSRLSISKDNSKSQVFLKMNSLQTDDTA |
| | | RYYCARDRVTATLYAMDYWGQGTSVTVSS |

Humanized antibody C
C-H-1 antibody

| | | |
|---|---|---|
| SEQ ID NO: 1315 | HC CDR1 (Kabat) | AYGVN |
| SEQ ID NO: 1316 | HC CDR2 (Kabat) | MIWGDGNTDYNSALKS |
| SEQ ID NO: 1317 | HC CDR3 (Kabat) | DRVTATLYAMDY |
| SEQ ID NO: 1318 | HC CDR1 (Chothia) | GFSLTAY |
| SEQ ID NO: 1319 | HC CDR2 (Chothia) | WGDGN |
| SEQ ID NO: 1317 | HC CDR3 (Chothia) | DRVTATLYAMDY |
| SEQ ID NO: 1320 | HC CDR1 (Combined) | GFSLTAYGVN |
| SEQ ID NO: 1316 | HC CDR2 (Combined) | MIWGDGNTDYNSALKS |
| SEQ ID NO: 1317 | HC CDR3(Combined) | DRVTATLYAMDY |
| SEQ ID NO: 1321 | LC CDR1 (Kabat) | SASQGISNYLN |
| SEQ ID NO: 1322 | LC CDR2 (Kabat) | YTSSLHS |
| SEQ ID NO: 1323 | LC CDR3 (Kabat) | QQYSKLPRT |
| SEQ ID NO: 1321 | LC CDR1 (Chothia) | SASQGISNYLN |
| SEQ ID NO: 1322 | LC CDR2 (Chothia) | YTSSLHS |
| SEQ ID NO: 1323 | LC CDR3 (Chothia) | QQYSKLPRT |
| SEQ ID NO: 1321 | LC CDR1 (Combined) | SASQGISNYLN |
| SEQ ID NO: 1322 | LC CDR2 (Combined) | YTSSLHS |
| SEQ ID NO: 1323 | LC CDR3(Combined) | QQYSKLPRT |
| SEQ ID NO: 1324 | VL | DIQMTQSPSSLSASVGDRVTITCSASQGISNYL |
| | | NWYQQTPGKAPKLLIYYTSSLHSGVPSRFSGS |
| | | GSGTDYTFTISSLQPEDIATYYCQQYSKLPRT |
| | | FGQGTKLQIT |
| SEQ ID NO: 1325 | VH | QVQLQESGPGLVRPSQTLSLTCTVSGFSLTA |
| | | YGVNWVRQPPGRGLEWLGMIWGDGNTDY |
| | | NSALKSRVTMLKDTSKNQFSLRLSSVTAAD |
| | | TAVYYCARDRVTATLYAMDYW |
| | | GQGSLVTVSS |

Humanized antibody C Variable light chain (VL)

| | | | |
|---|---|---|---|
| SEQ ID NO: 3000 | VL | C-H-VL.1 | DIQMTQSPSFLSASVGDRVTITCSASQGISNY |
| | | | LNWYQQKPGKAVKLLIYYTSSLHSGVPSRFS |
| | | | GSGSGTEYTLTISSLQPEDFATYYCQQYSKL |
| | | | PRTFGGGTKVEIK |
| SEQ ID NO: 3001 | VL | C-H-VL.2 | DIQMTQSPSFLSASVGDRVTITCSASQGISNY |
| | | | LNWYQQKPGKAVKLLIYYTSSLHSGVPSRFS |
| | | | GSGSGTDYTLTISSLQPEDFATYYCQQYSKL |
| | | | PRTFGGGTKVEIK |
| SEQ ID NO: 3002 | VL | C-H-VL.3 | DIQMTQSPSSLSASVGDRVTITCSASQGISNY |
| | | | LNWYQQKPGKVVKLLIYYTSSLHSGVPSRFS |
| | | | GSGSGTDYTLTISSLQPEDVATYYCQQYSKL |
| | | | PRTFGGGTKVEIK |
| SEQ ID NO: 3003 | VL | C-H-VL.4 | DIQMTQSPSSLSASVGDRVTITCSASQGISNY |
| | | | LNWYQQKPGQAVKLLIYYTSSLHSGVPSRFS |
| | | | GSGSGTDYTLTISSLQPEDVATYYCQQYSKL |
| | | | PRTFGGGTKVEIK |
| SEQ ID NO: 3004 | VL | C-H-VL.5 | DIQMTQSPSSLSASVGDRVTITCSASQGISNY |
| | | | LNWYQQKPGKAVKLLIYYTSSLHSGVPSRFS |
| | | | GSGSGTDYTFTISSLQPEDIATYYCQQYSKLP |
| | | | RTFGGGTKVEIK |
| SEQ ID NO: 3005 | VL | C-H-VL.6 | DIQMTQSPSSLSASVGDRVTITCSASQGISNY |
| | | | LNWYQQKPGKTVKLLIYYTSSLHSGIPSRFS |
| | | | GSGSGTDYTLTIRSLQPEDFATYYCQQYSKL |
| | | | PRTFGGGTKVEIK |
| SEQ ID NO: 3006 | VL | C-H-VL.7 | AIQMTQSPSSLSASVGDRVTITCSASQGISNY |
| | | | LNWYQQKPGKAVKLLIYYTSSLHSGVPSRFS |
| | | | GSGSGTDYTLTISSLQPEDFATYYCQQYSKL |
| | | | PRTFGGGTKVEIK |
| SEQ ID NO: 3007 | VL | C-H-VL.8 | DIQMTQSPSSVSASVGDRVTITCSASQGISNY |
| | | | LNWYQQKPGKAVKLLIYYTSSLHSGVPSRFS |
| | | | GSGSGTDYTLTISSLQPEDFATYYCQQYSKL |
| | | | PRTFGGGTKVEIK |
| SEQ ID NO: 3008 | VL | C-H-VL.9 | DIQMTQSPSSLSASVGDRVTITCSASQGISNY |
| | | | LNWYQQKPGKAVKRLIYYTSSLHSGVPSRF |
| | | | SGSGSGTEYTLTISNLQPEDFATYYCQQYSK |
| | | | LPRTFGGGTKVEIK |
| SEQ ID NO: 3009 | VL | C-H-VL.10 | AIRMTQSPFSLSASVGDRVTITCSASQGISNY |
| | | | LNWYQQKPAKAVKLFIYYTSSLHSGVPSRFS |
| | | | GSGSGTDYTLTISSLQPEDFATYYCQQYSKL |
| | | | PRTFGGGTKVEIK |
| SEQ ID NO: 3010 | VL | C-H-VL.11 | DIQMTQSPSSLSASVGDRVTITCSASQGISNY |
| | | | LNWYQQKPGKAVKRLIYYTSSLHSGVPSRF |
| | | | SGSGSGTEYTLTISSLQPEDFATYYCQQYSK |
| | | | LPRTFGGGTKVEIK |
| SEQ ID NO: 3011 | VL | C-H-VL.12 | DIQMTQSPSTLSASVGDRVTITCSASQGISNY |
| | | | LNWYQQKPGKAVKLLIYYTSSLHSGVPSRFS |
| | | | GSGSGTEYTLTISSLQPDDFATYYCQQYSKL |
| | | | PRTFGGGTKVEIK |
| SEQ ID NO:3012 | VL | C-H-VL.13 | DIQMTQSPSSLSASVGDRVTITCSASQGISNY |
| | | | LNWYQQKPGKAVKSLIYYTSSLHSGVPSRFS |
| | | | GSGSGTDYTLTISSLQPEDFATYYCQQYSKL |
| | | | PRTFGGGTKVEIK |
| SEQ ID NO: 3013 | VL | C-H-VL.14 | DIQMTQSPSSLSASVGDRVTITCSASQGISNY |
| | | | LNWYQQKPGKAVKSLIYYTSSLHSGVPSKFS |
| | | | GSGSGTDYTLTISSLQPEDFATYYCQQYSKL |
| | | | PRTFGGGTKVEIK |
| SEQ ID NO: 3014 | VL | C-H-VL.15 | DIQMTQSPSSLSASVGDRVTITCSASQGISNY |
| | | | LNWYQQKPEKAVKSLIYYTSSLHSGVPSRFS |
| | | | GSGSGTDYTLTISSLQPEDFATYYCQQYSKL |
| | | | PRTFGGGTKVEIK |
| SEQ ID NO: 3015 | VL | C-H-VL.16 | DIQMTQSPSAMSASVGDRVTITCSASQGISN |
| | | | YLNWYQQKPGKVVKRLIYYTSSLHSGVPSR |
| | | | FSGSGSGTEYTLTISSLQPEDFATYYCQQYSK |
| | | | LPRTFGGGTKVEIK |
| SEQ ID NO: 3016 | VL | C-H-VL.17 | DIVMTQSPDSLAVSLGERATINCSASQGISNY |
| | | | LNWYQQKPGQPVKLLIYYTSSLHSGVPDRFS |
| | | | GSGSGTDYTLTISSLQAEDVAVYYCQQYSK |
| | | | LPRTFGGGTKVEIK |
| SEQ ID NO: 3017 | VL | C-H-VL.18 | EIVMTQSPGTLSLSPGERATLSCSASQGISNY |
| | | | LNWYQQKPGQAVKLLIYYTSSLHSGIPDRFS |
| | | | GSGSGTDYTLTISRLEPEDFAVYYCQQYSKL |
| | | | PRTFGGGTKVEIK |
| SEQ ID NO: 3018 | VL | C-H-VL.19 | EIVMTQSPPTLSLSPGERVTLSCSASQGISNY |
| | | | LNWYQQKPGQAVKLLIYYTSSLHSGIPARFS |
| | | | GSGSGTDYTLTISSLQPEDFAVYYCQQYSKL |
| | | | PRTFGGGTKVEIK |
| SEQ ID NO: 3019 | VL | C-H-VL.20 | EIVMTQSPPTLSLSPGERVTLSCSASQGISNY |
| | | | LNWYQQKPGQAVKLLIYYTSSLHSSIPARFS |
| | | | GSGSGTDYTLTISSLQPEDFAVYYCQQYSKL |
| | | | PRTFGGGTKVEIK |
| SEQ ID NO: 3020 | VL | C-H-VL.21 | EIVMTQSPATLSLSPGERATLSCSASQGISNY |
| | | | LNWYQQKPGQAVKLLIYYTSSLHSGIPARFS |
| | | | GSGSGTDYTLTISSLEPEDFAVYYCQQYSKL |
| | | | PRTFGGGTKVEIK |
| SEQ ID NO: 3021 | VL | C-H-VL.22 | EIVMTQSPATLSLSPGERATLSCSASQGISNY |
| | | | LNWYQQKPGQAVKLLIYYTSSLHSGIPARFS |
| | | | GSGSGTDYTLTISRLEPEDFAVYYCQQYSKL |
| | | | PRTFGGGTKVEIK |
| SEQ ID NO: 3022 | VL | C-H-VL.23 | EIVMTQSPATLSLSPGERATLSCSASQGISNY |
| | | | LNWYQQKPGQAVKLLIYYTSSLHSGIPDRFS |
| | | | GSGSGTDYTLTISRLEPEDFAVYYCQQYSKL |
| | | | PRTFGGGTKVEIK |
| SEQ ID NO: 3023 | VL | C-H-VL.24 | EIVMTQSPATLSLSPGERATLSCSASQGISNY |
| | | | LNWYQQKPGLAVKLLIYYTSSLHSGIPDRFS |
| | | | GSGSGTDYTLTISRLEPEDFAVYYCQQYSKL |
| | | | PRTFGGGTKVEIK |
| SEQ ID NO: 3024 | VL | C-H-VL.25 | DIQMIQSPSFLSASVGDRVSIICSASQGISNYL |
| | | | NWYLQKPGKSVKLFIYYTSSLHSGVSSRFSG |
| | | | RGSGTDYTLTIISLKPEDFAAYYCQQYSKLP |
| | | | RTFGGGTKVEIK |
| SEQ ID NO: 3025 | VL | C-H-VL.26 | EIVMTQSPATLSLSPGERATLSCSASQGISNY |
| | | | LNWYQQKPGQAVKLLIYYTSSLHSGIPARFS |
| | | | GSGSGTDYTLTISSLQPEDFAVYYCQQYSKL |
| | | | PRTFGGGTKVEIK |
| SEQ ID NO: 3026 | VL | C-H-VL.27 | EIVMTQSPATLSLSPGERATLSCSASQGISNY |
| | | | LNWYQQKPGQAVKLLIYYTSSLHSGIPARFS |
| | | | GSGPGTDYTLTISSLEPEDFAVYYCQQYSKL |
| | | | PRTFGGGTKVEIK |
| SEQ ID NO: 3027 | VL | C-H-VL.28 | DIVMTQTPLSLSVTPGQPASISCSASQGISNY |
| | | | LNWYLQKPGQSVKLLIYYTSSLHSGVPDRFS |
| | | | GSGSGTDYTLKISRVEAEDVGVYYCQQYSK |
| | | | LPRTFGGGTKVEIK |
| SEQ ID NO: 3028 | VL | C-H-VL.29 | DIVMTQTPLSLSVTPGQPASISCSASQGISNY |
| | | | LNWYLQKPGQPVKLLIYYTSSLHSGVPDRFS |
| | | | GSGSGTDYTLKISRVEAEDVGVYYCQQYSK |
| | | | LPRTFGGGTKVEIK |
| SEQ ID NO: 3029 | VL | C-H-VL.30 | DIVMTQSPAFLSVTPGEKVTITCSASQGISNY |
| | | | LNWYQQKPDQAVKLLIYYTSSLHSGVPSRFS |
| | | | GSGSGTDYTFTISSLEAEDAATYYCQQYSKL |
| | | | PRTFGGGTKVEIK |
| SEQ ID NO: 3030 | VL | C-H-VL.31 | DIVMTQSPLSLPVTPGEPASISCSASQGISNYL |
| | | | NWYLQKPGQSVKLLIYYTSSLHSGVPDRFSG |
| | | | SGSGTDYTLKISRVEAEDVGVYYCQQYSKL |
| | | | PRTFGGGTKVEIK |
| SEQ ID NO: 3031 | VL | C-H-VL.32 | DIVMTQTPLSLPVTPGEPASISCSASQGISNY |
| | | | LNWYLQKPGQSVKLLIYYTSSLHSGVPDRFS |
| | | | GSGSGTDYTLKISRVEAEDVGVYYCQQYSK |
| | | | LPRTFGGGTKVEIK |
| SEQ ID NO: 3032 | VL | C-H-VL.33 | EIVMTQSPATLSVSPGERATLSCSASQGISNY |
| | | | LNWYQQKPGQAVKLLIYYTSSLHSGIPARFS |
| | | | GSGSGTEYTLTISILQSEDFAVYYCQQYSKLP |
| | | | RTFGGGTKVEIK |
| SEQ ID NO: 3033 | VL | C-H-VL.34 | EIVMTQSPATLSVSPGERATLSCSASQGISNY |
| | | | LNWYQQKPGQAVKLLIYYTSSLHSGIPARFS |
| | | | GSGSGTEYTLTISSLQSEDFAVYYCQQYSKL |
| | | | PRTFGGGTKVEIK |
| SEQ ID NO: 3034 | VL | C-H-VL.35 | DIVMTQSPLSLPVTLGQPASISCSASQGISNY |
| | | | LNWYQQRPGQSVKRLIYYTSSLHSGVPDRFS |
| | | | GSGSGTDYTLKISRVEAEDVGVYYCQQYSK |
| | | | LPRTFGGGTKVEIK |
| SEQ ID NO: 3035 | VL | C-H-VL.36 | EITMTQSPAFMSATPGDKVNISCSASQGISNY |
| | | | LNWYQQKPGEAVKFIIYYTSSLHSGIPPRFSG |
| | | | SGYGTDYTLTINNIESEDAAYYYCQQYSKLP |
| | | | RTFGGGTKVEIK |
| SEQ ID NO: 3036 | VL | C-H-VL.37 | DIVMTQTPLSSPVTLGQPASISCSASQGISNY |
| | | | LNWYQQRPGQPVKLLIYYTSSLHSGVPDRFS |
| | | | GSGAGTDYTLKISRVEAEDVGVYYCQQYSK |
| | | | LPRTFGGGTKVEIK |
| SEQ ID NO: 3037 | VL | C-H-VL.38 | EIVMTQSPDFQSVTPKEKVTITCSASQGISNY |
| | | | LNWYQQKPDQSVKLLIYYTSSLHSGVPSRFS |
| | | | GSGSGTDYTLTINSLEAEDAATYYCQQYSKL |
| | | | PRTFGGGTKVEIK |
| SEQ ID NO: 3038 | VL | C-H-VL.39 | EIVMTQTPLSLSITPGEQASISCSASQGISNYL |
| | | | NWYLQKARPVVKLLIYYTSSLHSGVPDRFS |
| | | | GSGSGTDYTLKISRVEAEDFGVYYCQQYSK |
| | | | LPRTFGGGTKVEIK |
| SEQ ID NO: 3039 | VL | C-H-VL.40 | EIVMTQTPLSLSITPGEQASMSCSASQGISNY |
| | | | LNWYLQKARPVVKLLIYYTSSLHSGVPDRFS |
| | | | GSGSGTDYTLKISRVEAEDFGVYYCQQYSK |
| | | | LPRTFGGGTKVEIK |

Humanized antibody C Variable HEAVY chain (VH)

| | | | |
|---|---|---|---|
| SEQ ID NO: 3040 | VH | C-H-VH.1 | QVTLKESGPVLVKPTETLTLTCTVSGFSLTA |
| | | | YGVNWVRQPPGKALEWLGMIWGDGNTDY |
| | | | NSALKSRLTISKDNSKSQVVLTMTNMDPVD |
| | | | TATYYCARDRVTATLYAMDYWGQGTLVTV |
| | | | SS |
| SEQ ID NO: 3041 | VH | C-H-VH.2 | QVTLKESGPALVKPTETLTLTCTVSGFSLTA |
| | | | YGVNWVRQPPGKALEWLGMIWGDGNTDY |
| | | | NSALKSRLIISKDNSKSQVVLTMTNMDPVDT |
| | | | ATYYCARDRVTATLYAMDYWGQGTLVTVS |
| | | | S |
| SEQ ID NO: 3042 | VH | C-H-VH.3 | QVTLKESGPALVKPTQTLTLTCTVSGFSLTA |
| | | | YGVNWVRQPPGKALEWLGMIWGDGNTDY |
| | | | NSALKSRLTISKDNSKSQVVLTMTNMDPVD |
| | | | TATYYCARDRVTATLYAMDYWGQGTLVTV |
| | | | SS |
| SEQ ID NO: 3043 | VH | C-H-VH.4 | QVQLQESGPGLVKPSGTLSLTCAVSGFSLTA |
| | | | YGVNWVRQPPGKGLEWLGMIWGDGNTDY |
| | | | NSALKSRLTISKDNSKSQVSLKLSSVTAADT |
| | | | AVYYCARDRVTATLYAMDYWGQGTLVTV |
| | | | SS |
| SEQ ID NO: 3044 | VH | C-H-VH.5 | QVTLKESGPTLVKPTQTLTLTCTVSGFSLTA |
| | | | YGVNWVRQPPGKALEWLGMIWGDGNTDY |
| | | | NSALKSRLTITKDNSKSQVVLTMTNMDPVD |
| | | | TATYYCARDRVTATLYAMDYWGQGTLVTV |
| | | | SS |
| SEQ ID NO: 3045 | VH | C-H-VH.6 | QVTLKESGPALVKPTQTLTLTCTVSGFSLTA |
| | | | YGVNWVRQPPGKALEWLGMIWGDGNTDY |
| | | | NSALKSRLTITKDNSKSQVVLTMTNMDPVD |
| | | | TATYYCARDRVTATLYAMDYWGQGTLVTV |
| | | | SS |
| SEQ ID NO: 3046 | VH | C-H-VH.7 | QVQLQESGPGLVKPSQTLSLTCTVSGFSLTA |
| | | | YGVNWVRQPPGKGLEWLGMIWGDGNTDY |
| | | | NSALKSRLTISKDNSKSQVSLKLSSVTAADT |
| | | | AVYYCARDRVTATLYAMDYWGQGTLVTV |
| | | | SS |
| SEQ ID NO: 3047 | VH | C-H-VH.8 | QVQLQESGPGLVKPSETLSLTCTVSGFSLTA |
| | | | YGVNWVRQPPGKGLEWLGMIWGDGNTDY |
| | | | NSALKSRLTISKDNSKSQVSLKLSSVTAADT |
| | | | AVYYCARDRVTATLYAMDYWGQGTLVTV |
| | | | SS |
| SEQ ID NO: 3048 | VH | C-H-VH.9 | QVQLQESGPGLVKPSQTLSLTCAVSGFSLTA |
| | | | YGVNWVRQPPGKGLEWLGMIWGDGNTDY |
| | | | NSALKSRLTISKDNSKSQVSLKLSSVTAADT |
| | | | AVYYCARDRVTATLYAMDYWGQGTLVTV |
| | | | SS |
| SEQ ID NO: 3049 | VH | C-H-VH.10 | QVQLQESGPGLVKPSDTLSLTCTVSGFSLTA |
| | | | YGVNWVRQPPGKGLEWLGMIWGDGNTDY |
| | | | NSALKSRLTISKDNSKSQVSLKLSSVTAADT |
| | | | AVYYCARDRVTATLYAMDYWGQGTLVTV |
| | | | SS |
| SEQ ID NO: 3050 | VH | C-H-VH.11 | QVQLQESGPGLVKPSQTLSLTCTVSGFSLTA |
| | | | YGVNWVRQHPGKGLEWLGMIWGDGNTDY |
| | | | NSALKSRLTISKDNSKSQVSLKLSSVTAADT |
| | | | AVYYCARDRVTATLYAMDYWGQGTLVTV |
| | | | SS |
| SEQ ID NO: 3051 | VH | C-H-VH.12 | QVQLQESGPGLVKPSQTLSLTCTVSGFSLTA |
| | | | YGVNWVRQPAGKGLEWLGMIWGDGNTDY |
| | | | NSALKSRLTISKDNSKSQVSLKLSSVTAADT |
| | | | AVYYCARDRVTATLYAMDYWGQGTLVTV |
| | | | SS |
| SEQ ID NO: 3052 | VH | C-H-VH.13 | QVQLQESGPGLVKPSQTLSLTCAVSGFSLTA |
| | | | YGVNWVRQPPGKGLEWLGMIWGDGNTDY |
| | | | NSALKSRLTISKDNSKSQVSLKLSSVTAVDT |
| | | | AVYYCARDRVTATLYAMDYWGQGTLVTV |
| | | | SS |
| SEQ ID NO: 3053 | VH | C-H-VH.14 | QVQLQESGPGLVKPSETLSLTCTVSGFSLTA |
| | | | YGVNWVRQPPGKGLEWLGMIWGDGNTDY |
| | | | NSALKSRLTISKDNSKSHVSLKLSSVTAADT |
| | | | AVYYCARDRVTATLYAMDYWGQGTLVTV |
| | | | SS |
| SEQ ID NO: 3054 | VH | C-H-VH.15 | QVQLQESGPGLVKPSETLSLTCAVSGFSLTA |
| | | | YGVNWVRQPPGKGLEWLGMIWGDGNTDY |
| | | | NSALKSRLTISKDNSKSQVSLKLSSVTAADT |
| | | | AVYYCARDRVTATLYAMDYWGQGTLVTV |
| | | | SS |
| SEQ ID NO: 3055 | VH | C-H-VH.16 | QVQLQESGPGLVKPSQTLSLTCAVYGFSLTA |
| | | | YGVNWVRQPPGKGLEWLGMIWGDGNTDY |
| | | | NSALKSRLTISKDNSKSQVSLKLSSVTAADT |
| | | | AVYYCARDRVTATLYAMDYWGQGTLVTV |
| | | | SS |
| SEQ ID NO: 3056 | VH | C-H-VH.17 | RVQLQESGPGLVKPSETLSLTCTVSGFSLTA |
| | | | YGVNWVRQPPGKGLEWLGMIWGDGNTDY |
| | | | NSALKSRLTISKDNSKSQVPLKLSSVTAADT |
| | | | AVYYCARDRVTATLYAMDYWGQGTLVTV |
| | | | SS |
| SEQ ID NO: 3057 | VH | C-H-VH.18 | QVQLQESGPGLVKPSQTLSLTCTVSGFSLTA |
| | | | YGVNWVRQHPGKGLEWLGMIWGDGNTDY |
| | | | NSALKSLLTISKDNSKSQVSLKLSSVTAADT |
| | | | AVYYCARDRVTATLYAMDYWGQGTLVTV |
| | | | SS |
| SEQ ID NO: 3058 | VH | C-H-VH.19 | QVQLQESGPGLVKPSDTLSLTCAVSGFSLTA |
| | | | YGVNWVRQPPGKGLEWLGMIWGDGNTDY |
| | | | NSALKSRLTISKDNSKSQVSLKLSSVTALDT |
| | | | AVYYCARDRVTATLYAMDYWGQGTLVTV |
| | | | SS |
| SEQ ID NO: 3059 | VH | C-H-VH.20 | QVQLQESGPGLVKPSDTLSLTCAVSGFSLTA |
| | | | YGVNWVRQPPGKGLEWLGMIWGDGNTDY |
| | | | NSALKSRLTISKDNSKSQVSLKLSSVTAVDT |
| | | | AVYYCARDRVTATLYAMDYWGQGTLVTV |
| | | | SS |
| SEQ ID NO: 3060 | VH | C-H-VH.21 | QVQLQESGSGLVKPSQTLSLTCAVSGFSLTA |
| | | | YGVNWVRQPPGKGLEWLGMIWGDGNTDY |
| | | | NSALKSRLTISKDNSKSQVSLKLSSVTAADT |
| | | | AVYYCARDRVTATLYAMDYWGQGTLVTV |
| | | | SS |
| SEQ ID NO: 3061 | VH | C-H-VH.22 | EVOLVESGGGLVQPGRSLRLSCTVSGFSLTA |
| | | | YGVNWVRQAPGKGLEWLGMIWGDGNTDY |
| | | | NSALKSRLTISKDNSKSIVYLQMNSLKTEDT |
| | | | AVYYCARDRVTATLYAMDYWGQGTLVTV |
| | | | SS |
| SEQ ID NO: 3062 | VH | C-H-VH.23 | EVOLVESGGGLVQPGPSLRLSCTVSGFSLTA |
| | | | YGVNWVRQAPGKGLEWLGMIWGDGNTDY |
| | | | NSALKSRLTISKDNSKSIVYLQMNSLKTEDT |
| | | | AVYYCARDRVTATLYAMDYWGQGTLVTV |
| | | | SS |
| SEQ ID NO: 3063 | VH | C-H-VH.24 | QVQLQESGSGLVKPSQTLSLTCAVSGFSLTA |
| | | | YGVNWVRQSPGKGLEWLGMIWGDGNTDY |
| | | | NSALKSRLTISKDNSKSQVSLKLSSVTAADT |
| | | | AVYYCARDRVTATLYAMDYWGQGTLVTV |
| | | | SS |
| SEQ ID NO: 3064 | VH | C-H-VH.25 | QVQLQESGPGLVKPSETLSLTCTVSGFSLTA |
| | | | YGVNWVRQPAGKGLEWLGMIWGDGNTDY |
| | | | NSALKSRLTISKDNSKSQVSLKLSSVTAADT |
| | | | AVYYCARDRVTATLYAMDYWGQGTLVTV |
| | | | SS |
| SEQ ID NO: 3065 | VH | C-H-VH.26 | EVOLVESGGGLVKPGRSLRLSCTVSGFSLTA |
| | | | YGVNWVRQAPGKGLEWLGMIWGDGNTDY |
| | | | NSALKSRLTISKDNSKSIVYLQMNSLKTEDT |
| | | | AVYYCARDRVTATLYAMDYWGQGTLVTV |
| | | | SS |
| SEQ ID NO: 3066 | VH | C-H-VH.27 | QVQLQESGPGLVKPSETLSLTCAVYGFSLTA |
| | | | YGVNWVRQPPGKGLEWLGMIWGDGNTDY |
| | | | NSALKSRLTISKDNSKSQVYLKLSSVTAADT |
| | | | AVYYCARDRVTATLYAMDYWGQGTLVTV |
| | | | SS |
| SEQ ID NO: 3067 | VH | C-H-VH.28 | QVQLQESGPGLVKPSDTLSLTCAVSGFSLTA |
| | | | YGVNWVRQPPGKGLEWLGMIWGDGNTDY |
| | | | NSALKSRLTISKDNSKSQVSLKLSSVTAVDT |
| | | | GVYYCARDRVTATLYAMDYWGQGTLVTV |
| | | | SS |
| SEQ ID NO: 3068 | VH | C-H-VH.29 | EVQLVESGGGLVQPGGSLRLSCAVSGFSLTA |
| | | | YGVNWVRQAPGKGLEWLGMIWGDGNTDY |
| | | | NSALKSRLTISKDNSKSSVYLQMNSLKTEDT |
| | | | AVYYCARDRVTATLYAMDYWGQGTLVTV |
| | | | SS |
| SEQ ID NO: 3069 | VH | C-H-VH.30 | EVOLVESGGGLVKPGGSLRLSCAVSGFSLTA |
| | | | YGVNWVRQAPGKGLEWLGMIWGDGNTDY |
| | | | NSALKSRLTISKDNSKSTVYLQMNSLKTEDT |
| | | | AVYYCARDRVTATLYAMDYWGQGTLVTV |
| | | | SS |
| SEQ ID NO: 3070 | VH | C-H-VH.31 | QVQLQQSGPGLVKPSQTLSLTCAVSGFSLTA |
| | | | YGVNWVRQSPSRGLEWLGMIWGDGNTDY |
| | | | NSALKSRLTINKDNSKSQVSLQLNSVTPEDT |
| | | | AVYYCARDRVTATLYAMDYWGQGTLVTV |
| | | | SS |
| SEQ ID NO: 3071 | VH | C-H-VH.32 | QVQLVESGGGLVQPGGSLRLSCSVSGFSLTA |
| | | | YGVNWVRQAPGKGLEWLGMIWGDGNTDY |
| | | | NSALKSRLTISKDNSKSTVYLQMNSLRAEDT |
| | | | AVYYCARDRVTATLYAMDYWGQGTLVTV |
| | | | SS |
| SEQ ID NO: 3072 | VH | C-H-VH.33 | QVQLQQWGAGLLKPSETLSLTCAVYGFSLT |
| | | | AYGVNWVRQPPGKGLEWLGMIWGDGNTD |
| | | | YNSALKSRLTISKDNSKSQVSLKLSSVTAAD |
| | | | TAVYYCARDRVTATLYAMDYWGQGTLVT |
| | | | VSS |
| SEQ ID NO: 3073 | VH | C-H-VH.34 | QVQLVESGGGVVQPGRSLRLSCAVSGFSLT |
| | | | AYGVNWVRQAPGKGLEWLGMIWGDGNTD |
| | | | YNSALKSRLTISKDNSTSTVFLQMNSLRAED |
| | | | TAVYYCARDRVTATLYAMDYWGQGTLVT |
| | | | VSS |
| SEQ ID NO: 3074 | VH | C-H-VH.35 | EVOLVESGGGLVQPGGSLRLSCAVSGFSLTA |
| | | | YGVNWVRQAPGKGLEWLGMIWGDGNTDY |
| | | | NSALKSRLTISKDNSKSTVYLQMNSLRAEDT |
| | | | AVYYCARDRVTATLYAMDYWGQGTLVTV |
| | | | SS |
| SEQ ID NO: 3075 | VH | C-H-VH.36 | EVOLVESGGGLVQPGGSLRLSCAVSGFSLTA |
| | | | YGVNWVRQAPGKGLEWLGMIWGDGNTDY |
| | | | NSALKSRLTISKDNAKSSVYLQMNSLRDEDT |
| | | | AVYYCARDRVTATLYAMDYWGQGTLVTV |
| | | | SS |
| SEQ ID NO: 3076 | VH | C-H-VH.37 | EVQLLESGGGLVQPGGSLRLSCAVSGFSLTA |
| | | | YGVNWVRQAPGKGLEWLGMIWGDGNTDY |
| | | | NSALKSRLTISKDNSKSTVYLQMNSLRAEDT |
| | | | AVYYCARDRVTATLYAMDYWGQGTLVTV |
| | | | SS |
| SEQ ID NO: 3077 | VH | C-H-VH.38 | QVQLVESGGGLVKPGGSLRLSCAVSGFSLT |
| | | | AYGVNWVRQAPGKGLEWLGMIWGDGNTD |
| | | | YNSALKSRLTISKDNAKSSVYLQMNSLRAE |
| | | | DTAVYYCARDRVTATLYAMDYWGQGTLV |
| | | | TVSS |
| SEQ ID NO: 3078 | VH | C-H-VH.39 | EVOLVESGGGLVQPGGSLKLSCAVSGFSLTA |
| | | | YGVNWVRQASGKGLEWLGMIWGDGNTDY |
| | | | NSALKSRLTISKDNSKSTVYLQMNSLKTEDT |
| | | | AVYYCARDRVTATLYAMDYWGQGTLVTV |
| | | | SS |
| SEQ ID NO: 3079 | VH | C-H-VH.40 | QVQLLESGGGLVKPGGSLRLSCAVSGFSLTA |
| | | | YGVNWVRQAPGKGLEWLGMIWGDGNTDY |
| | | | NSALKSRLTISKDNAKSSVYLQMNSLRAEDT |
| | | | AVYYCARDRVTATLYAMDYWGQGTLVTV |
| | | | SS |
| SEQ ID NO: 3080 | VH | C-H-VH.41 | QVQLVESGGGVVQPGRSLRLSCAVSGFSLT |
| | | | AYGVNWVRQAPGKGLEWLGMIWGDGNTD |
| | | | YNSALKSRLTISKDNSKSTVYLQMNSLRAED |
| | | | TAVYYCARDRVTATLYAMDYWGQGTLVT |
| | | | VSS |
| SEQ ID NO: 3081 | VH | C-H-VH.42 | QVQLVESGGGVVQPGRSLRLSCAVSGFSLT |
| | | | AYGVNWVRQAPGKGLEWLGMIWGDGNTD |
| | | | YNSALKSRLTISKDNSKSRVYLQMNSLRAE |
| | | | DTAVYYCARDRVTATLYAMDYWGQGTLV |
| | | | TVSS |
| SEQ ID NO: 3082 | VH | C-H-VH.43 | QVQLVESGGGVVQPGRSLRLSCAVSGFSLT |
| | | | AYGVNWVRQAPGKGLEWLGMIWGDGNTD |
| | | | YNSALKSRLAISKDNSKSTVYLQMNSLRAE |
| | | | DTAVYYCARDRVTATLYAMDYWGQGTLV |
| | | | TVSS |
| SEQ ID NO: 3083 | VH | C-H-VH.44 | QVQLVESGGGVVQPGGSLRLSCAVSGFSLT |
| | | | AYGVNWVRQAPGKGLEWLGMIWGDGNTD |
| | | | YNSALKSRLTISKDNSKSTVYLQMNSLRAED |
| | | | TAVYYCARDRVTATLYAMDYWGQGTLVT |
| | | | VSS |
| SEQ ID NO: 3084 | VH | C-H-VH.45 | EVOLVESGGGLVQPGGSLRLSCAVSGFSLTA |
| | | | YGVNWVRQAPGKGLEWLGMIWGDGNTDY |
| | | | NSALKSRLTISKDNAKSTVYLQMNSLRAED |
| | | | TAVYYCARDRVTATLYAMDYWGQGTLVT |
| | | | VSS |
| SEQ ID NO: 3085 | VH | C-H-VH.46 | EVOLVESGGGLVQPGGSLRLSCAVSGFSLTA |
| | | | YGVNWVRQAPGKGLEWLGMIWGDGNTDY |
| | | | NSALKSRLTISKDNAKSSVYLQMNSLRAEDT |
| | | | AVYYCARDRVTATLYAMDYWGQGTLVTV |
| | | | SS |
| SEQ ID NO: 3086 | VH | C-H-VH.47 | EVOLVESGGVVVQPGGSLRLSCAVSGFSLT |
| | | | AYGVNWVRQAPGKGLEWLGMIWGDGNTD |
| | | | YNSALKSRLTISKDNSKSSVYLQMNSLRTED |
| | | | TALYYCARDRVTATLYAMDYWGQGTLVTV |
| | | | SS |
| SEQ ID NO: 3087 | VH | C-H-VH.48 | EVOLVESGGGLVQPGGSLRLSCAVSGFSLTA |
| | | | YGVNWVRQAPGKGLEWLGMIWGDGNTDY |
| | | | NSALKSRLTISKHNSKSTVYLQMNSLRAEDT |
| | | | AVYYCARDRVTATLYAMDYWGQGTLVTV |
| | | | SS |
| SEQ ID NO: 3088 | VH | C-H-VH.49 | EVOLVESGGGLVKPGGSLRLSCAVSGFSLTA |
| | | | YGVNWVRQAPGKGLEWLGMIWGDGNTDY |
| | | | NSALKSRLTISKDNAKSSVYLQMNSLRAEDT |
| | | | AVYYCARDRVTATLYAMDYWGQGTLVTV |
| | | | SS |
| SEQ ID NO: 3089 | VH | C-H-VH.50 | EVOLVESGGGLIQPGGSLRLSCAVSGFSLTA |
| | | | YGVNWVRQPPGKGLEWLGMIWGDGNTDY |
| | | | NSALKSRLTISKDNSKSTVYLQMNSLRAEDT |
| | | | AVYYCARDRVTATLYAMDYWGQGTLVTV |
| | | | SS |

Exemplary anti-TCRβ V5 antibodies of the disclosure are provided in Table 11. In some embodiments, the anti-TCRβ V5 is antibody E, e.g., humanized antibody E (antibody E-H), as provided in Table 11. In some embodiments, the anti-TCRβV antibody comprises one or more (e.g., all three) of a LC CDR1, LC CDR2, and LC CDR3 provided in Table 11; and/or one or more (e.g., all three) of a HC CDR1, HC CDR2, and HC CDR3 provided in Table 11, or a sequence with at least 95 identity thereto. In some embodiments, antibody E comprises a variable heavy chain (VH) and/or a variable light chain (VL) provided in Table 11, or a sequence with at least 95% identity thereto.

In some embodiments, antibody E comprises a heavy chain comprising the amino acid sequence of SEQ ID NO: 3284 and/or a light chain comprising the amino acid sequence of SEQ ID NO: 3285, or a sequence with at least 95% identity thereto.

TABLE 11
Amino acid sequences for anti TCRβ V5 antibodies
Amino acid and nucleotide sequences for murine and humanized antibody molecules which bind to
TCRVB 5 (e.g., TCRVB 5-5 or TCRVB 5-6). The amino acid the heavy and light chain CDRs, and
the amino acid and nucleotide sequences of the heavy and light chain variable regions, and
the heavy and light chains are shown.
Murine antibody E, also referred to as MH3-2

| | | |
|---|---|---|
| SEQ ID NO: 1298 | HC CDR1 (Kabat) | SSWMN |
| SEQ ID NO: 1299 | HC CDR2 (Kabat) | RIYPGDGDTKYNGKFKG |
| SEQ ID NO: 1300 | HC CDR3 (Kabat) | RGTGGWYFDV |
| SEQ ID NO: 1302 | HC CDR1 (Chothia) | GYAFSSS |
| SEQ ID NO: 1303 | HC CDR2 (Chothia) | YPGDGD |
| SEQ ID NO: 1301 | HC CDR3 (Chothia) | RGTGGWYFDV |
| SEQ ID NO: 1304 | HC CDR1 (Combined) | GYAFSSSWMN |
| SEQ ID NO: 1299 | HC CDR2 (Combined)) | RIYPGDGDTKYNGKFKG |
| SEQ ID NO: 1301 | HC CDR3(Combined) | RGTGGWYFDV |
| SEQ ID NO: 1305 | LC CDR1 (Kabat) | RASESVDSSGNSFMH |
| SEQ ID NO: 1306 | LC CDR2 (Kabat) | RASNLES |
| SEQ ID NO: 1307 | LC CDR3 (Kabat) | QQSFDDPFT |
| SEQ ID NO: 1308 | LC CDR1 (Chothia) | SESVDSSGNSF |
| SEQ ID NO: 1306 | LC CDR2 (Chothia) | RASNLES |
| SEQ ID NO: 1307 | LC CDR3 (Chothia) | QQSFDDPFT |
| SEQ ID NO: 1305 | LC CDR1 (Combined) | RASESVDSSGNSFMH |
| SEQ ID NO: 1306 | LC CDR2 (Combined) | RASNLES |
| SEQ ID NO: 1307 | LC CDR3(Combined) | QQSFDDPFT |
| SEQ ID NO: 3091 | VH | QVQLQQSGPELVKPGASVKISCKASGYAFSSS |
| | | WMNWVKQRPGQGLEWIGRIYPGDGDTKYN |
| | | GKFKGKATLTADKSSSTAYMHLSSLTSVDSA |
| | | VYFCARRGTGGWYFDVWGAGTTVTVSS |
| SEQ ID NO: 3284 | Heavy chain | METDTLLLWVLLLWVPGSTGQVQLQQSGPEL |
| | | VKPGASVKISCKASGYAFSSSWMNWVKQRP |
| | | GQGLEWIGRIYPGDGDTKYNGKFKGKATLTA |
| | | DKSSSTAYMHLSSLTSVDSAVYFCARRGTGG |
| | | WYFDVWGAGTTVTVSSAKTTAPSVYPLAPV |
| | | CGDTTGSSVTLGCLVKGYFPEPVTLTWNSGS |
| | | LSSGVHTFPAVLQSDLYTLSSSVTVTSSTWPS |
| | | QSITCNVAHPASSTKVDKKIEPRGPTIKPCPPC |
| | | KCPAPNLLGGPSVFIFPPKIKDVLMISLSPIVTC |
| | | VVVDVSEDDPDVQISWFVNNVEVHTAQTQT |
| | | HREDYNSTLRVVSALPIQHQDWMSGKEFKCK |
| | | VNNKDLPAPIERTISKPKGSVRAPQVYVLPPPE |
| | | EEMTKKQVTLTCMVTDFMPEDIYVEWTNNG |
| | | KTELNYKNTEPVLDSDGSYFMYSKLRVEKKN |
| | | WVERNSYSCSVVHEGLHNHHTTKSFSRTPGK |
| SEQ ID NO: 3092 | VL | DIVLTQSPASLAVSLGQRATISCRASESVDSSG |
| | | NSFMHWYQQKPGQPPQLLIYRASNLESGIPAR |
| | | FSGSGSRTDFTLTINPVEADDVATFYCQQSFD |
| | | DPFTFGSGTKLEIK |
| SEQ ID NO: 3285 | Light chain | METDTLLLWVLLLWVPGSTGDIVLTQSPASL |
| | | AVSLGQRATISCRASESVDSSGNSFMHWYQQ |
| | | KPGQPPQLLIYRASNLESGIPARFSGSGSRTDF |
| | | TLTINPVEADDVATFYCQQSFDDPFTFGSGTK |
| | | LEIKRADAAPTVSIFPPSSEQLTSGGASVVCFL |
| | | NNFYPKDINVKWKIDGSERQNGVLNSWTDQ |
| | | DSKDSTYSMSSTLTLTKDEYERHNSYTCEAT |
| | | HKTSTSPIVKSFNRNEC |

Humanized antibody E (E-H antibody)
Variable light chain (VL)

| | | | |
|---|---|---|---|
| SEQ ID NO: 3093 | VL | E-H.1 | DIVLTQSPDSLAVSLGERATINCRASESVDSSG |
| | | | NSFMHWYQQKPGQPPQLLIYRASNLESGVPD |
| | | | RFSGSGSRTDFTLTISSLQAEDVAVYYCQQSF |
| | | | DDPFTFGQGTKLEIK |
| SEQ ID NO: 3094 | VL | E-H.2 | EIVLTQSPATLSLSPGERATLSCRASESVDSSG |
| | | | NSFMHWYQQKPGQAPQLLIYRASNLESGIPA |
| | | | RFSGSGSRTDFTLTISSLEPEDFAVYYCQQSFD |
| | | | DPFTFGQGTKLEIK |
| SEQ ID NO: 3095 | VL | E-H.3 | EIVLTQSPATLSLSPGERATLSCRASESVDSSG |
| | | | NSFMHWYQQKPGQAPQLLIYRASNLESGIPA |
| | | | RFSGSGSRTDFTLTISRLEPEDFAVYYCQQSFD |
| | | | DPFTFGQGTKLEIK |
| SEQ ID NO: 3096 | VL | E-H.4 | EIVLTQSPATLSLSPGERATLSCRASESVDSSG |
| | | | NSFMHWYQQKPGQAPQLLIYRASNLESGIPA |
| | | | RFSGSGSRTDFTLTISSLQPEDFAVYYCQQSFD |
| | | | DPFTFGQGTKLEIK |
| SEQ ID NO: 3097 | VL | E-H.5 | DIQLTQSPSSLSASVGDRVTITCRASESVDSSG |
| | | | NSFMHWYQQKPGQAPQLLIYRASNLESGVPS |
| | | | RFSGSGSRTDFTLTISSLQPEDVATYYCQQSFD |
| | | | DPFTFGQGTKLEIK |
| SEQ ID NO: 3098 | VL | E-H.6 | EIVLTQSPATLSLSPGERATLSCRASESVDSSG |
| | | | NSFMHWYQQKPGQAPQLLIYRASNLESGIPA |
| | | | RFSGSGPRTDFTLTISSLEPEDFAVYYCQQSFD |
| | | | DPFTFGQGTKLEIK |
| SEQ ID NO: 3099 | VL | E-H.7 | EIVLTQSPATLSLSPGERATLSCRASESVDSSG |
| | | | NSFMHWYQQKPGQAPQLLIYRASNLESGIPD |
| | | | RFSGSGSRTDFTLTISRLEPEDFAVYYCQQSFD |
| | | | DPFTFGQGTKLEIK |
| SEQ ID NO: 3100 | VL | E-H.8 | DIQLTQSPSSLSASVGDRVTITCRASESVDSSG |
| | | | NSFMHWYQQKPGKVPQLLIYRASNLESGVPS |
| | | | RFSGSGSRTDFTLTISSLQPEDVATYYCQQSFD |
| | | | DPFTFGQGTKLEIK |
| SEQ ID NO: 3101 | VL | E-H.9 | DIQLTQSPSSLSASVGDRVTITCRASESVDSSG |
| | | | NSFMHWYQQKPGKTPQLLIYRASNLESGIPSR |
| | | | FSGSGSRTDFTLTIRSLQPEDFATYYCQQSFDD |
| | | | PFTFGQGTKLEIK |
| SEQ ID NO: 3102 | VL | E-H.10 | EIVLTQSPGTLSLSPGERATLSCRASESVDSSG |
| | | | NSFMHWYQQKPGQAPQLLIYRASNLESGIPD |
| | | | RFSGSGSRTDFTLTISRLEPEDFAVYYCQQSFD |
| | | | DPFTFGQGTKLEIK |
| SEQ ID NO: 3103 | VL | E-H.11 | EIVLTQSPATLSLSPGERATLSCRASESVDSSG |
| | | | NSFMHWYQQKPGLAPQLLIYRASNLESGIPDR |
| | | | FSGSGSRTDFTLTISRLEPEDFAVYYCQQSFDD |
| | | | PFTFGQGTKLEIK |
| SEQ ID NO: 3104 | VL | E-H.12 | DIQLTQSPSSLSASVGDRVTITCRASESVDSSG |
| | | | NSFMHWYQQKPGKAPQLLIYRASNLESGVPS |
| | | | RFSGSGSRTDFTLTISSLQPEDFATYYCQQSFD |
| | | | DPFTFGQGTKLEIK |
| SEQ ID NO: 3105 | VL | E-H.13 | DIQLTQSPSSVSASVGDRVTITCRASESVDSSG |
| | | | NSFMHWYQQKPGKAPQLLIYRASNLESGVPS |
| | | | RFSGSGSRTDFTLTISSLQPEDFATYYCQQSFD |
| | | | DPFTFGQGTKLEIK |
| SEQ ID NO: 3106 | VL | E-H.14 | AIQLTQSPSSLSASVGDRVTITCRASESVDSSG |
| | | | NSFMHWYQQKPGKAPQLLIYRASNLESGVPS |
| | | | RFSGSGSRTDFTLTISSLQPEDFATYYCQQSFD |
| | | | DPFTFGQGTKLEIK |
| SEQ ID NO: 3107 | VL | E-H.15 | DIQLTQSPSFLSASVGDRVTITCRASESVDSSG |
| | | | NSFMHWYQQKPGKAPQLLIYRASNLESGVPS |
| | | | RFSGSGSRTEFTLTISSLQPEDFATYYCQQSFD |
| | | | DPFTFGQGTKLEIK |
| SEQ ID NO: 3108 | VL | E-H.16 | DIQLTQSPSSLSASVGDRVTITCRASESVDSSG |
| | | | NSFMHWYQQKPGKAPQLLIYRASNLESGVPS |
| | | | RFSGSGSRTDFTFTISSLQPEDIATYYCQQSFD |
| | | | DPFTFGQGTKLEIK |
| SEQ ID NO: 3109 | VL | E-H.17 | EIVLTQSPATLSVSPGERATLSCRASESVDSSG |
| | | | NSFMHWYQQKPGQAPQLLIYRASNLESGIPA |
| | | | RFSGSGSRTEFTLTISILQSEDFAVYYCQQSFD |
| | | | DPFTFGQGTKLEIK |
| SEQ ID NO: 3110 | VL | E-H.18 | EIVLTQSPATLSVSPGERATLSCRASESVDSSG |
| | | | NSFMHWYQQKPGQAPQLLIYRASNLESGIPA |
| | | | RFSGSGSRTEFTLTISSLQSEDFAVYYCQQSFD |
| | | | DPFTFGQGTKLEIK |
| SEQ ID NO: 3111 | VL | E-H.19 | AIRLTQSPFSLSASVGDRVTITCRASESVDSSG |
| | | | NSFMHWYQQKPAKAPQLFIYRASNLESGVPS |
| | | | RFSGSGSRTDFTLTISSLQPEDFATYYCQQSFD |
| | | | DPFTFGQGTKLEIK |
| SEQ ID NO: 3112 | VL | E-H.20 | DIQLTQSPSSLSASVGDRVTITCRASESVDSSG |
| | | | NSFMHWYQQKPGKAPQSLIYRASNLESGVPS |
| | | | RFSGSGSRTDFTLTISSLQPEDFATYYCQQSFD |
| | | | DPFTFGQGTKLEIK |
| SEQ ID NO: 3113 | VL | E-H.21 | DIQLTQSPSSLSASVGDRVTITCRASESVDSSG |
| | | | NSFMHWYQQKPGKAPQRLIYRASNLESGVPS |
| | | | RFSGSGSRTEFTLTISNLQPEDFATYYCQQSFD |
| | | | DPFTFGQGTKLEIK |
| SEQ ID NO: 3114 | VL | E-H.22 | DIQLTQSPSTLSASVGDRVTITCRASESVDSSG |
| | | | NSFMHWYQQKPGKAPQLLIYRASNLESGVPS |
| | | | RFSGSGSRTEFTLTISSLQPDDFATYYCQQSFD |
| | | | DPFTFGQGTKLEIK |
| SEQ ID NO: 3115 | VL | E-H.23 | EIVLTQSPDFQSVTPKEKVTITCRASESVDSSG |
| | | | NSFMHWYQQKPDQSPQLLIYRASNLESGVPS |
| | | | RFSGSGSRTDFTLTINSLEAEDAATYYCQQSF |
| | | | DDPFTFGQGTKLEIK |
| SEQ ID NO: 3116 | VL | E-H.24 | DIQLTQSPSSLSASVGDRVTITCRASESVDSSG |
| | | | NSFMHWYQQKPGKAPQSLIYRASNLESGVPS |
| | | | KFSGSGSRTDFTLTISSLQPEDFATYYCQQSFD |
| | | | DPFTFGQGTKLEIK |
| SEQ ID NO: 3117 | VL | E-H.25 | DIQLTQSPSSLSASVGDRVTITCRASESVDSSG |
| | | | NSFMHWYQQKPGKAPQRLIYRASNLESGVPS |
| | | | RFSGSGSRTEFTLTISSLQPEDFATYYCQQSFD |
| | | | DPFTFGQGTKLEIK |
| SEQ ID NO: 3118 | VL | E-H.26 | DIVLTQTPLSLSVTPGQPASISCRASESVDSSG |
| | | | NSFMHWYLQKPGQPPQLLIYRASNLESGVPD |
| | | | RFSGSGSRTDFTLKISRVEAEDVGVYYCQQSF |
| | | | DDPFTFGQGTKLEIK |
| SEQ ID NO: 3119 | VL | E-H.27 | DIQLTQSPSSLSASVGDRVTITCRASESVDSSG |
| | | | NSFMHWYQQKPEKAPQSLIYRASNLESGVPS |
| | | | RFSGSGSRTDFTLTISSLQPEDFATYYCQQSFD |
| | | | DPFTFGQGTKLEIK |
| SEQ ID NO: 3120 | VL | E-H.28 | EIVLTQSPPTLSLSPGERVTLSCRASESVDSSG |
| | | | NSFMHWYQQKPGQAPQLLIYRASNLESGIPA |
| | | | RFSGSGSRTDFTLTISSLQPEDFAVYYCQQSFD |
| | | | DPFTFGQGTKLEIK |
| SEQ ID NO: 3121 | VL | E-H.29 | DIQLTQSPSAMSASVGDRVTITCRASESVDSS |
| | | | GNSFMHWYQQKPGKVPQRLIYRASNLESGVP |
| | | | SRFSGSGSRTEFTLTISSLQPEDFATYYCQQSF |
| | | | DDPFTFGQGTKLEIK |
| SEQ ID NO:3122 | VL | E-H.30 | DIVLTQSPLSLPVTPGEPASISCRASESVDSSGN |
| | | | SFMHWYLQKPGQSPQLLIYRASNLESGVPDR |
| | | | FSGSGSRTDFTLKISRVEAEDVGVYYCQQSFD |
| | | | DPFTFGQGTKLEIK |
| SEQ ID NO: 3123 | VL | E-H.31 | DIVLTQTPLSLPVTPGEPASISCRASESVDSSG |
| | | | NSFMHWYLQKPGQSPQLLIYRASNLESGVPD |
| | | | RFSGSGSRTDFTLKISRVEAEDVGVYYCQQSF |
| | | | DDPFTFGQGTKLEIK |
| SEQ ID NO: 3124 | VL | E-H.32 | DIVLTQTPLSLSVTPGQPASISCRASESVDSSG |
| | | | NSFMHWYLQKPGQSPQLLIYRASNLESGVPD |
| | | | RFSGSGSRTDFTLKISRVEAEDVGVYYCQQSF |
| | | | DDPFTFGQGTKLEIK |
| SEQ ID NO: 3125 | VL | E-H.33 | EIVLTQSPPTLSLSPGERVTLSCRASESVDSSG |
| | | | NSFMHWYQQKPGQAPQLLIYRASNLESSIPAR |
| | | | FSGSGSRTDFTLTISSLQPEDFAVYYCQQSFDD |
| | | | PFTFGQGTKLEIK |
| SEQ ID NO: 3126 | VL | E-H.34 | DIVLTQSPLSLPVTLGQPASISCRASESVDSSG |
| | | | NSFMHWYQQRPGQSPQRLIYRASNLESGVPD |
| | | | RFSGSGSRTDFTLKISRVEAEDVGVYYCQQSF |
| | | | DDPFTFGQGTKLEIK |
| SEQ ID NO: 3127 | VL | E-H.35 | DIVLTQTPLSSPVTLGQPASISCRASESVDSSG |
| | | | NSFMHWYQQRPGQPPQLLIYRASNLESGVPD |
| | | | RFSGSGARTDFTLKISRVEAEDVGVYYCQQSF |
| | | | DDPFTFGQGTKLEIK |
| SEQ ID NO: 3128 | VL | E-H.36 | DIVLTQSPAFLSVTPGEKVTITCRASESVDSSG |
| | | | NSFMHWYQQKPDQAPQLLIYRASNLESGVPS |
| | | | RFSGSGSRTDFTFTISSLEAEDAATYYCQQSFD |
| | | | DPFTFGQGTKLEIK |
| SEQ ID NO: 3129 | VL | E-H.37 | DIQLIQSPSFLSASVGDRVSIICRASESVDSSGN |
| | | | SFMHWYLQKPGKSPQLFIYRASNLESGVSSRF |
| | | | SGRGSRTDFTLTIISLKPEDFAAYYCQQSFDDP |
| | | | FTFGQGTKLEIK |
| SEQ ID NO: 3130 | VL | E-H.38 | EIVLTQTPLSLSITPGEQASISCRASESVDSSGN |
| | | | SFMHWYLQKARPVPQLLIYRASNLESGVPDR |
| | | | FSGSGSRTDFTLKISRVEAEDFGVYYCQQSFD |
| | | | DPFTFGQGTKLEIK |
| SEQ ID NO: 3131 | VL | E-H.39 | EIVLTQTPLSLSITPGEQASMSCRASESVDSSG |
| | | | NSFMHWYLQKARPVPQLLIYRASNLESGVPD |
| | | | RFSGSGSRTDFTLKISRVEAEDFGVYYCQQSF |
| | | | DDPFTFGQGTKLEIK |
| SEQ ID NO: 3132 | VL | E-H.40 | EITLTQSPAFMSATPGDKVNISCRASESVDSSG |
| | | | NSFMHWYQQKPGEAPQFIIYRASNLESGIPPR |
| | | | FSGSGYRTDFTLTINNIESEDAAYYYCQQSFD |
| | | | DPFTFGQGTKLEIK |

Variable HEAVY chain (VH)

| | | | |
|---|---|---|---|
| SEQ ID NO: 3133 | VH | E-H.1 | QVQLVQSGAEVKKPGASVKVSCKASGYAFSS |
| | | | SWMNWVRQAPGQGLEWIGRIYPGDGDTKYN |
| | | | GKFKGRATLTADKSTSTAYMELSSLRSEDTA |
| | | | VYYCARRGTGGWYFDVWGQGTTVTVSS |
| SEQ ID NO: 3134 | VH | E-H.2 | QVQLVQSGAEVKKPGSSVKVSCKASGYAFSS |
| | | | SWMNWVRQAPGQGLEWIGRIYPGDGDTKYN |
| | | | GKFKGRATLTADKSTSTAYMELSSLRSEDTA |
| | | | VYYCARRGTGGWYFDVWGQGTTVTVSS |
| SEQ ID NO: 3135 | VH | E-H.3 | QVQLVQSGAEVKKPGASVKVSCKASGYAFSS |
| | | | SWMNWVRQAPGKGLEWIGRIYPGDGDTKYN |
| | | | GKFKGRATLTADKSTSTAYMELSSLRSEDTA |
| | | | VYYCARRGTGGWYFDVWGQGTTVTVSS |
| SEQ ID NO: 3136 | VH | E-H.4 | QVQLVQSGAEVKKPGASVKVSCKASGYAFSS |
| | | | SWMNWVRQAPGQELEWIGRIYPGDGDTKYN |
| | | | GKFKGRATLTADKSISTAYMELSSLRSEDTAT |
| | | | YYCARRGTGGWYFDVWGQGTTVTVSS |
| SEQ ID NO: 3137 | VH | E-H.5 | EVQLVQSGAEVKKPGATVKISCKASGYAFSSS |
| | | | WMNWVQQAPGKGLEWIGRIYPGDGDTKYN |
| | | | GKFKGRATLTADKSTSTAYMELSSLRSEDTA |
| | | | VYYCARRGTGGWYFDVWGQGTTVTVSS |
| SEQ ID NO: 3138 | VH | E-H.6 | QVQLVQSGAEVKKTGSSVKVSCKASGYAFSS |
| | | | SWMNWVRQAPGQALEWIGRIYPGDGDTKYN |
| | | | GKFKGRATLTADKSMSTAYMELSSLRSEDTA |
| | | | MYYCARRGTGGWYFDVWGQGTTVTVSS |
| SEQ ID NO: 3139 | VH | E-H.7 | QVQLVQSGAEVKKPGASVKVSCKASGYAFSS |
| | | | SWMNWVRQAPGQRLEWIGRIYPGDGDTKYN |
| | | | GKFKGRATLTADKSASTAYMELSSLRSEDMA |
| | | | VYYCARRGTGGWYFDVWGQGTTVTVSS |
| SEQ ID NO: 3140 | VH | E-H.8 | QVQLVQSGAEVKKPGASVKVSCKASGYAFSS |
| | | | SWMNWVRQAPGQGLEWIGRIYPGDGDTKYN |
| | | | GKFKGRATLTADKSTSTAYMELRSLRSDDMA |
| | | | VYYCARRGTGGWYFDVWGQGTTVTVSS |
| SEQ ID NO: 3141 | VH | E-H.9 | QVQLVQSGAEVKKPGASVKVSCKASGYAFSS |
| | | | SWMNWVRQAPGQRLEWIGRIYPGDGDTKYN |
| | | | GKFKGRATLTADKSASTAYMELSSLRSEDTA |
| | | | VYYCARRGTGGWYFDVWGQGTTVTVSS |
| SEQ ID NO: 3142 | VH | E-H.10 | QVQLVQSGAEVKKPGASVKVSCKASGYAFSS |
| | | | SWMNWVRQAPGQGLEWIGRIYPGDGDTKYN |
| | | | GKFKGRATLTADKSTSTAYMELRSLRSDDTA |
| | | | VYYCARRGTGGWYFDVWGQGTTVTVSS |
| SEQ ID NO: 3143 | VH | E-H.11 | QVQLVQSGAEVKKPGASVKVSCKASGYAFSS |
| | | | SWMNWVRQAPGQGLEWIGRIYPGDGDTKYN |
| | | | GKFKGRATLTADKSISTAYMELSRLRSDDTA |
| | | | VYYCARRGTGGWYFDVWGQGTTVTVSS |
| SEQ ID NO: 3144 | VH | E-H.12 | QVQLVQSGAEVKKPGASVKVSCKASGYAFSS |
| | | | SWMNWVRQAPGQGLEWIGRIYPGDGDTKYN |
| | | | GKFKGRATLTADKSISTAYMELSRLRSDDTV |
| | | | VYYCARRGTGGWYFDVWGQGTTVTVSS |
| SEQ ID NO: 3145 | VH | E-H.13 | QVQLVQSGAEVKKPGASVKVSCKASGYAFSS |
| | | | SWMNWVRQAPGQGLEWIGRIYPGDGDTKYN |
| | | | GKFKGWATLTADKSISTAYMELSRLRSDDTA |
| | | | VYYCARRGTGGWYFDVWGQGTTVTVSS |
| SEQ ID NO: 3146 | VH | E-H.14 | QVQLVQSGAEVKKPGASVKVSCKASGYAFSS |
| | | | SWMNWVRQATGQGLEWIGRIYPGDGDTKYN |
| | | | GKFKGRATLTANKSISTAYMELSSLRSEDTAV |
| | | | YYCARRGTGGWYFDVWGQGTTVTVSS |
| SEQ ID NO: 3147 | VH | E-H.15 | QVQLVQSGSELKKPGASVKVSCKASGYAFSS |
| | | | SWMNWVRQAPGQGLEWIGRIYPGDGDTKYN |
| | | | GKFKGRAVLSADKSVSTAYLQISSLKAEDTA |
| | | | VYYCARRGTGGWYFDVWGQGTTVTVSS |
| SEQ ID NO: 3148 | VH | E-H.16 | QVQLVQSGPEVKKPGTSVKVSCKASGYAFSS |
| | | | SWMNWVRQARGQRLEWIGRIYPGDGDTKYN |
| | | | GKFKGRATLTADKSTSTAYMELSSLRSEDTA |
| | | | VYYCARRGTGGWYFDVWGQGTTVTVSS |
| SEQ ID NO: 3149 | VH | E-H.17 | EVQLVQSGAEVKKPGESLKISCKASGYAFSSS |
| | | | WMNWVRQMPGKGLEWIGRIYPGDGDTKYN |
| | | | GKFKGQATLSADKSISTAYLQWSSLKASDTA |
| | | | MYYCARRGTGGWYFDVWGQGTTVTVSS |
| SEQ ID NO: 3150 | VH | E-H.18 | QVQLVQSGSELKKPGASVKVSCKASGYAFSS |
| | | | SWMNWVRQAPGQGLEWIGRIYPGDGDTKYN |
| | | | GKFKGRAVLSADKSVSMAYLQISSLKAEDTA |
| | | | VYYCARRGTGGWYFDVWGQGTTVTVSS |
| SEQ ID NO: 3151 | VH | E-H.19 | QVQLVQSGHEVKQPGASVKVSCKASGYAFSS |
| | | | SWMNWVPQAPGQGLEWIGRIYPGDGDTKYN |
| | | | GKFKGRAVLSADKSASTAYLQISSLKAEDMA |
| | | | MYYCARRGTGGWYFDVWGQGTTVTVSS |
| SEQ ID NO: 3152 | VH | E-H.20 | EVQLVQSGAEVKKPGESLKISCKASGYAFSSS |
| | | | WMNWVRQMPGKGLEWIGRIYPGDGDTKYN |
| | | | GKFKGQATLSADKPISTAYLQWSSLKASDTA |
| | | | MYYCARRGTGGWYFDVWGQGTTVTVSS |
| SEQ ID NO: 3153 | VH | E-H.21 | EVQLVQSGAEVKKPGESLRISCKASGYAFSSS |
| | | | WMNWVRQMPGKGLEWIGRIYPGDGDTKYN |
| | | | GKFKGQATLSADKSISTAYLQWSSLKASDTA |
| | | | MYYCARRGTGGWYFDVWGQGTTVTVSS |
| SEQ ID NO: 3154 | VH | E-H.22 | EVQLVQSGAEVKKPGESLRISCKASGYAFSSS |
| | | | WMNWVRQMPGKGLEWIGRIYPGDGDTKYN |
| | | | GKFKGHATLSADKSISTAYLQWSSLKASDTA |
| | | | MYYCARRGTGGWYFDVWGQGTTVTVSS |
| SEQ ID NO: 3155 | VH | E-H.23 | QVQLVQSGAEVKKTGSSVKVSCKASGYAFSS |
| | | | SWMNWVRQAPRQALEWIGRIYPGDGDTKYN |
| | | | GKFKGRATLTADKSMSTAYMELSSLRSEDTA |
| | | | MYYCARRGTGGWYFDVWGQGTTVTVSS |
| SEQ ID NO: 3156 | VH | E-H.24 | EVQLVESGGGLVQPGRSLRLSCTASGYAFSSS |
| | | | WMNWVRQAPGKGLEWIGRIYPGDGDTKYN |
| | | | GKFKGRATLSADKSKSIAYLQMNSLKTEDTA |
| | | | VYYCARRGTGGWYFDVWGQGTTVTVSS |
| SEQ ID NO: 3157 | VH | E-H.25 | EVQLVESGGGLVQPGPSLRLSCTASGYAFSSS |
| | | | WMNWVRQAPGKGLEWIGRIYPGDGDTKYN |
| | | | GKFKGRATLSADKSKSIAYLQMNSLKTEDTA |
| | | | VYYCARRGTGGWYFDVWGQGTTVTVSS |
| SEQ ID NO: 3158 | VH | E-H.26 | QVQLQESGPGLVKPSQTLSLTCTASGYAFSSS |
| | | | WMNWVRQPPGKGLEWIGRIYPGDGDTKYNG |
| | | | KFKGRATLSADKSKSQASLKLSSVTAADTAV |
| | | | YYCARRGTGGWYFDVWGQGTTVTVSS |
| SEQ ID NO: 3159 | VH | E-H.27 | QVQLQESGPGLVKPSGTLSLTCAASGYAFSSS |
| | | | WMNWVRQPPGKGLEWIGRIYPGDGDTKYNG |
| | | | KFKGRATLSADKSKSQASLKLSSVTAADTAV |
| | | | YYCARRGTGGWYFDVWGQGTTVTVSS |
| SEQ ID NO: 3160 | VH | E-H.28 | EVQLVESGGGLVKPGRSLRLSCTASGYAFSSS |
| | | | WMNWVRQAPGKGLEWIGRIYPGDGDTKYN |
| | | | GKFKGRATLSADKSKSIAYLQMNSLKTEDTA |
| | | | VYYCARRGTGGWYFDVWGQGTTVTVSS |
| SEQ ID NO: 3161 | VH | E-H.29 | EVQLVESGGGLVQPGGSLKLSCAASGYAFSSS |
| | | | WMNWVRQASGKGLEWIGRIYPGDGDTKYN |
| | | | GKFKGRATLSADKSKSTAYLQMNSLKTEDTA |
| | | | VYYCARRGTGGWYFDVWGQGTTVTVSS |
| SEQ ID NO: 3162 | VH | E-H.30 | QVQLQESGPGLVKPSQTLSLTCAASGYAFSSS |
| | | | WMNWVRQPPGKGLEWIGRIYPGDGDTKYNG |
| | | | KFKGRATLSADKSKSQASLKLSSVTAADTAV |
| | | | YYCARRGTGGWYFDVWGQGTTVTVSS |
| SEQ ID NO: 3163 | VH | E-H.31 | EVQLVESGGGLVKPGGSLRLSCAASGYAFSSS |
| | | | WMNWVRQAPGKGLEWIGRIYPGDGDTKYN |
| | | | GKFKGRATLSADKSKSTAYLQMNSLKTEDTA |
| | | | VYYCARRGTGGWYFDVWGQGTTVTVSS |
| SEQ ID NO: 3164 | VH | E-H.32 | EVQLVESGGALVKPGGSLRLSCAASGYAFSSS |
| | | | WMNWVRQAPGKGLEWIGRIYPGDGDTKYN |
| | | | GKFKGRATLSADKSKSTAYLQMNSLKTEDTA |
| | | | VYYCARRGTGGWYFDVWGQGTTVTVSS |
| SEQ ID NO: 3165 | VH | E-H.33 | QVQLQESGPGLVKPSQTLSLTCAAYGYAFSSS |
| | | | WMNWVRQPPGKGLEWIGRIYPGDGDTKYNG |
| | | | KFKGRATLSADKSKSQASLKLSSVTAADTAV |
| | | | YYCARRGTGGWYFDVWGQGTTVTVSS |
| SEQ ID NO: 3166 | VH | E-H.34 | QVQLQESGSGLVKPSQTLSLTCAASGYAFSSS |
| | | | WMNWVRQPPGKGLEWIGRIYPGDGDTKYNG |
| | | | KFKGRATLSADKSKSQASLKLSSVTAADTAV |
| | | | YYCARRGTGGWYFDVWGQGTTVTVSS |
| SEQ ID NO: 3167 | VH | E-H.35 | EVQLVESGGGLVQPGGSLRLSCAASGYAFSSS |
| | | | WMNWVRQAPGKGLEWIGRIYPGDGDTKYN |
| | | | GKFKGRATLSADKSKSSAYLQMNSLKTEDTA |
| | | | VYYCARRGTGGWYFDVWGQGTTVTVSS |
| SEQ ID NO: 3168 | VH | E-H.36 | QVQLQESGPGLVKPSDTLSLTCTASGYAFSSS |
| | | | WMNWVRQPPGKGLEWIGRIYPGDGDTKYNG |
| | | | KFKGRATLSADKSKSQASLKLSSVTAADTAV |
| | | | YYCARRGTGGWYFDVWGQGTTVTVSS |
| SEQ ID NO: 3169 | VH | E-H.37 | QVQLQESGPGLVKPSQTLSLTCTASGYAFSSS |
| | | | WMNWVRQHPGKGLEWIGRIYPGDGDTKYN |
| | | | GKFKGRATLSADKSKSQASLKLSSVTAADTA |
| | | | VYYCARRGTGGWYFDVWGQGTTVTVSS |
| SEQ ID NO: 3170 | VH | E-H.38 | QVQLQESGPGLVKPSQTLSLTCTASGYAFSSS |
| | | | WMNWVRQHPGKGLEWIGRIYPGDGDTKYN |
| | | | GKFKGLATLSADKSKSQASLKLSSVTAADTA |
| | | | VYYCARRGTGGWYFDVWGQGTTVTVSS |
| SEQ ID NO: 3171 | VH | E-H.39 | QVQLVESGGGVVQPGRSLRLSCAASGYAFSS |
| | | | SWMNWVRQAPGKGLEWIGRIYPGDGDTKYN |
| | | | GKFKGRATLSADKSKSTAYLQMSSLRAEDTA |
| | | | VYYCARRGTGGWYFDVWGQGTTVTVSS |
| SEQ ID NO: 3172 | VH | E-H.40 | QVQLVESGGGLVKPGGSLRLSCAASGYAFSS |
| | | | SWMNWVRQAPGKGLEWIGRIYPGDGDTKYN |
| | | | GKFKGRATLSADKAKSSAYLQMNSLRAEDT |
| | | | AVYYCARRGTGGWYFDVWGQGTTVTVSS |
| SEQ ID NO: 3173 | VH | E-H.41 | QVQLVESGGGLVQPGGSLRLSCSASGYAFSSS |
| | | | WMNWVRQAPGKGLEWIGRIYPGDGDTKYN |
| | | | GKFKGRATLSADKSKSTAYLQMNSLRAEDTA |
| | | | VYYCARRGTGGWYFDVWGQGTTVTVSS |
| SEQ ID NO: 3174 | VH | E-H.42 | QVQLLESGGGLVKPGGSLRLSCAASGYAFSSS |
| | | | WMNWVRQAPGKGLEWIGRIYPGDGDTKYN |
| | | | GKFKGRATLSADKAKSSAYLQMNSLRAEDT |
| | | | AVYYCARRGTGGWYFDVWGQGTTVTVSS |
| SEQ ID NO: 3175 | VH | E-H.43 | EVQLVESGGGLVQPGGSLRLSCSASGYAFSSS |
| | | | WMNWVRQAPGKGLEWIGRIYPGDGDTKYN |
| | | | GKFKGRATLSADKSKSTAYLQMSSLRAEDTA |
| | | | VYYCARRGTGGWYFDVWGQGTTVTVSS |
| SEQ ID NO: 3176 | VH | E-H.44 | QVQLQESGPGLVKPSDTLSLTCAASGYAFSSS |
| | | | WMNWVRQPPGKGLEWIGRIYPGDGDTKYNG |
| | | | KFKGRATLSADKSKSQASLKLSSVTAVDTAV |
| | | | YYCARRGTGGWYFDVWGQGTTVTVSS |
| SEQ ID NO: 3177 | VH | E-H.45 | QVQLQESGPGLVKPSQTLSLTCAASGYAFSSS |
| | | | WMNWVRQPPGKGLEWIGRIYPGDGDTKYNG |
| | | | KFKGRATLSADKSKSQASLKLSSVTAVDTAV |
| | | | YYCARRGTGGWYFDVWGQGTTVTVSS |
| SEQ ID NO: 3178 | VH | E-H.46 | EVQLVESGGGLVQPGGSLRLSCSASGYAFSSS |
| | | | WMNWVRQAPGKGLEWIGRIYPGDGDTKYN |
| | | | GKFKGRATLSADKSKSTAYVQMSSLRAEDTA |
| | | | VYYCARRGTGGWYFDVWGQGTTVTVSS |
| SEQ ID NO: 3179 | VH | E-H.47 | QVQLVDSGGGVVQPGRSLRLSCAASGYAFSS |
| | | | SWMNWVRQAPGKGLEWIGRIYPGDGDTKYN |
| | | | GKFKGRATLSADKSKSTAYLQMNSLRAEDTA |
| | | | VYYCARRGTGGWYFDVWGQGTTVTVSS |
| SEQ ID NO: 3180 | VH | E-H.48 | QVQLVESGGGVVQPGRSLRLSCAASGYAFSS |
| | | | SWMNWVRQAPGKGLEWIGRIYPGDGDTKYN |
| | | | GKFKGRATLSADKSKSTAYLQMNSLRAEGTA |
| | | | VYYCARRGTGGWYFDVWGQGTTVTVSS |
| SEQ ID NO: 3181 | VH | E-H.49 | QVQLVESGGGVVQPGRSLRLSCAASGYAFSS |
| | | | SWMNWVRQAPGKGLEWIGRIYPGDGDTKYN |
| | | | GKFKGRATLSADKSKSTAYLQMNSLRAEDTA |
| | | | VYYCARRGTGGWYFDVWGQGTTVTVSS |
| SEQ ID NO: 3182 | VH | E-H.50 | EVQLVESGGGLVQPGGSLRLSCAASGYAFSSS |
| | | | WMNWVRQAPGKGLEWIGRIYPGDGDTKYN |
| | | | GKFKGRATLSADKSKSTAYLQMNSLRAEDTA |
| | | | VYYCARRGTGGWYFDVWGQGTTVTVSS |

In some embodiments, the anti-TCRβ V5 antibody molecule comprises a VH and/or a VL of an antibody described in Table 33, or a sequence with at least 80%, 85%, 90%, 95%, 96%, 97%, 98%, 99% or more identity thereto.

In some embodiments, the anti-TCRβ V5 antibody molecule comprises a VH and a VL of an antibody described in Table 33, or a sequence with at least 80%, 85%, 90%, 95%, 96%, 97%, 98%, 99% or more identity thereto.

In some embodiments, the anti-TCRβ V5 antibody molecule comprises a VH and/or a VL of an antibody described in Table 11, or a sequence with at least 80%, 85%, 90%, 95%, 96%, 97%, 98%, 99% or more identity thereto.

In some embodiments, the anti-TCRβ V5 antibody molecule comprises a VH and a VL of an antibody described in Table 11, or a sequence with at least 80%, 85%, 90%, 95%, 96%, 97%, 98%, 99% or more identity thereto.

### Anti-TCRβ V10 Antibodies

Accordingly, in one aspect, the disclosure provides an anti-TCRβV antibody molecule that binds to a human TCRβ V10 subfamily member. In some embodiments, TCRβ V10 subfamily is also known as TCRβ V12. In some embodiments, the TCRβ V10 subfamily comprises: TCRβ V10-1*01, TCRβ V10-1*02, TCRβ V10-3*01 or TCRβ V10-2*01, or a variant thereof.

Exemplary anti-TCRβ V10 antibodies of the disclosure are provided in Table 12. In some embodiments, the anti-TCRβ V10 is antibody D, e.g., humanized antibody D (antibody D-H), as provided in Table 12. In some embodiments, antibody D comprises one or more (e.g., three) light chain CDRs and/or one or more (e.g., three) heavy chain CDRs provided in Table 12, or a sequence with at least 95% identity thereto. In some embodiments, antibody D comprises a variable heavy chain (VH) and/or a variable light chain (VL) provided in Table 12, or a sequence with at least 95% identity thereto.

TABLE 12
Amino acid sequences for anti TCRβ V10 antibodies
Amino acid and nucleotide sequences for murine and humanized antibody molecules which bind to
TCRBV 10 (e.g., TCRBV 10-1, TCRBV 10-2 or TCRBV 10-3). The amino acid the heavy and light
chain CDRs, and the amino acid and nucleotide sequences of the heavy and light chain variable regions,
and the heavy and light chains are shown.
Murine antibody D, also referred to as S511 antibody

| | | |
|---|---|---|
| SEQ ID NO: 1288 | HC CDR1 (Kabat) | SYGMS |
| SEQ ID NO: 1289 | HC CDR2 (Kabat) | LISSGGSYTYYTDSVKG |
| SEQ ID NO: 1290 | HC CDR3 (Kabat) | HGGNFFDY |
| SEQ ID NO: 1291 | HC CDR1 (Chothia) | GFTFRSY |
| SEQ ID NO: 1292 | HC CDR2 (Chothia) | SSGGSY |
| SEQ ID NO: 1290 | HC CDR3 (Chothia) | HGGNFFDY |
| SEQ ID NO: 1293 | HC CDR1 (Combined) | GFTFRSYGMS |
| SEQ ID NO: 1289 | HC CDR2 (Combined)) | LISSGGSYTYYTDSVKG |
| SEQ ID NO: 1290 | HC CDR3(Combined) | HGGNFFDY |
| SEQ ID NO: 1294 | LC CDR1 (Kabat) | SVSSSVSYMH |
| SEQ ID NO: 1295 | LC CDR2 (Kabat) | DTSKLAS |
| SEQ ID NO: 1296 | LC CDR3 (Kabat) | QQWSSNPQYT |
| SEQ ID NO: 1297 | LC CDR1 (Chothia) | SSSVSY |
| SEQ ID NO: 1295 | LC CDR2 (Chothia) | DTSKLAS |
| SEQ ID NO: 1296 | LC CDR3 (Chothia) | QQWSSNPQYT |
| SEQ ID NO: 1294 | LC CDR1 (Combined) | SVSSSVSYMH |
| SEQ ID NO: 1295 | LC CDR2 (Combined) | DTSKLAS |
| SEQ ID NO: 1296 | LC CDR3 (Combined) | QQWSSNPQYT |
| SEQ ID NO: 3183 | VH | EVQLVESGGDLVKPGGSLKLSCAVSGFTFRSY |
| | | GMSWVRQTPDKRLEWVALISSGGSYTYYTDS |
| | | VKGRFTISRDNAKNTLYLQMSSLKSEDTAIYY |
| | | CSRHGGNFFDYWGQGTTLTVSS |
| SEQ ID NO: 3184 | VL | QIVLTQSPSIMSASPGEKVTMTCSVSSSVSYM |
| | | HWYQQKSGTSPKRWIYDTSKLASGVPARFSG |
| | | SGSGTSYSLTISSMEAEDAATYYCQQWSSNPQ |
| | | YTFGGGTKLEIK |

Humanized antibody D (D-H antibody)
Variable light chain (VL)

| | | | |
|---|---|---|---|
| SEQ ID NO: 3185 | VL | D-VL- | DIVLTQSPAFLSVTPGEKVTITCSVSSSVSYMHWYQQK |
| | | H.1 | PDQAPKLLIYDTSKLASGVPSRFSGSGSGTDYTFTISSL |
| | | | EAEDAATYYCQQWSSNPQYTFGQGTKLEIK |
| SEQ ID NO: 3186 | VL | D-VL- | AIQLTQSPSSLSASVGDRVTITCSVSSSVSYMHWYQQK |
| | | H.2 | PGKAPKLLIYDTSKLASGVPSRFSGSGSGTDYTLTISSL |
| | | | QPEDFATYYCQQWSSNPQYTFGQGTKLEIK |
| SEQ ID NO: 3187 | VL | D-VL- | DIQLTQSPSFLSASVGDRVTITCSVSSSVSYMHWYQQK |
| | | H.3 | PGKAPKLLIYDTSKLASGVPSRFSGSGSGTEYTLTISSL |
| | | | QPEDFATYYCQQWSSNPQYTFGQGTKLEIK |
| SEQ ID NO: 3188 | VL | D-VL- | DIQLTQSPSSLSASVGDRVTITCSVSSSVSYMHWYQQK |
| | | H.4 | PGKAPKLLIYDTSKLASGVPSRFSGSGSGTDYTLTISSL |
| | | | QPEDFATYYCQQWSSNPQYTFGQGTKLEIK |
| SEQ ID NO: 3189 | VL | D-VL- | DIQLTQSPSSVSASVGDRVTITCSVSSSVSYMHWYQQ |
| | | H.5 | KPGKAPKLLIYDTSKLASGVPSRFSGSGSGTDYTLTISS |
| | | | LQPEDFATYYCQQWSSNPQYTFGQGTKLEIK |
| SEQ ID NO: 3190 | VL | D- VL- | DIQLTQSPSSLSASVGDRVTITCSVSSSVSYMHWYQQK |
| | | H.6 | PGKVPKLLIYDTSKLASGVPSRFSGSGSGTDYTLTISSL |
| | | | QPEDVATYYCQQWSSNPQYTFGQGTKLEIK |
| SEQ ID NO: 3191 | VL | D- VL- | DIQLTQSPSSLSASVGDRVTITCSVSSSVSYMHWYQQK |
| | | H.7 | PGQAPKLLIYDTSKLASGVPSRFSGSGSGTDYTLTISSL |
| | | | QPEDVATYYCQQWSSNPQYTFGQGTKLEIK |
| SEQ ID NO: 3192 | VL | D- VL- | EIVLTQSPDFQSVTPKEKVTITCSVSSSVSYMHWYQQK |
| | | H.8 | PDQSPKLLIYDTSKLASGVPSRFSGSGSGTDYTLTINSL |
| | | | EAEDAATYYCQQWSSNPQYTFGQGTKLEIK |
| SEQ ID NO: 3193 | VL | D- VL- | AIRLTQSPFSLSASVGDRVTITCSVSSSVSYMHWYQQK |
| | | H.9 | PAKAPKLFIYDTSKLASGVPSRFSGSGSGTDYTLTISSL |
| | | | QPEDFATYYCQQWSSNPQYTFGQGTKLEIK |
| SEQ ID NO: 3194 | VL | D- VL- | DIQLTQSPSSLSASVGDRVTITCSVSSSVSYMHWYQQK |
| | | H.10 | PGKAPKLLIYDTSKLASGVPSRFSGSGSGTDYTFTISSL |
| | | | QPEDIATYYCQQWSSNPQYTFGQGTKLEIK |
| SEQ ID NO: 3195 | VL | D- VL- | EIVLTQSPATLSLSPGERATLSCSVSSSVSYMHWYQQK |
| | | H.11 | PGQAPKLLIYDTSKLASGIPARFSGSGSGTDYTLTISSL |
| | | | EPEDFAVYYCQQWSSNPQYTFGQGTKLEIK |
| SEQ ID NO: 3196 | VL | D- VL- | DIQLTQSPSTLSASVGDRVTITCSVSSSVSYMHWYQQ |
| | | H.12 | KPGKAPKLLIYDTSKLASGVPSRFSGSGSGTEYTLTISS |
| | | | LQPDDFATYYCQQWSSNPQYTFGQGTKLEIK |
| SEQ ID NO: 3197 | VL | D- VL- | DIQLTQSPSSLSASVGDRVTITCSVSSSVSYMHWYQQK |
| | | H.13 | PGKTPKLLIYDTSKLASGIPSRFSGSGSGTDYTLTIRSL |
| | | | QPEDFATYYCQQWSSNPQYTFGQGTKLEIK |
| SEQ ID NO: 3198 | VL | D- VL- | EIVLTQSPPTLSLSPGERVTLSCSVSSSVSYMHWYQQK |
| | | H.14 | PGQAPKLLIYDTSKLASGIPARFSGSGSGTDYTLTISSL |
| | | | QPEDFAVYYCQQWSSNPQYTFGQGTKLEIK |
| SEQ ID NO: 3199 | VL | D- VL- | DIQLTQSPSSLSASVGDRVTITCSVSSSVSYMHWYQQK |
| | | H.15 | PGKAPKRLIYDTSKLASGVPSRFSGSGSGTEYTLTISSL |
| | | | QPEDFATYYCQQWSSNPQYTFGQGTKLEIK |
| SEQ ID NO: 3200 | VL | D- VL- | EIVLTQSPATLSLSPGERATLSCSVSSSVSYMHWYQQK |
| | | H.16 | PGQAPKLLIYDTSKLASGIPARFSGSGPGTDYTLTISSL |
| | | | EPEDFAVYYCQQWSSNPQYTFGQGTKLEIK |
| SEQ ID NO: 3201 | VL | D- VL- | EIVLTQSPATLSLSPGERATLSCSVSSSVSYMHWYQQK |
| | | H.17 | PGQAPKLLIYDTSKLASGIPARFSGSGSGTDYTLTISRL |
| | | | EPEDFAVYYCQQWSSNPQYTFGQGTKLEIK |
| SEQ ID NO: 3202 | VL | D- VL- | EIVLTQSPATLSLSPGERATLSCSVSSSVSYMHWYQQK |
| | | H.18 | PGQAPKLLIYDTSKLASGIPARFSGSGSGTDYTLTISSL |
| | | | QPEDFAVYYCQQWSSNPQYTFGQGTKLEIK |
| SEQ ID NO: 3203 | VL | D- VL- | EIVLTQSPATLSVSPGERATLSCSVSSSVSYMHWYQQ |
| | | H.19 | KPGQAPKLLIYDTSKLASGIPARFSGSGSGTEYTLTISS |
| | | | LQSEDFAVYYCQQWSSNPQYTFGQGTKLEIK |
| SEQ ID NO: 3204 | VL | D- VL- | EIVLTQSPATLSVSPGERATLSCSVSSSVSYMHWYQQ |
| | | H.20 | KPGQAPKLLIYDTSKLASGIPARFSGSGSGTEYTLTISIL |
| | | | QSEDFAVYYCQQWSSNPQYTFGQGTKLEIK |
| SEQ ID NO: 3205 | VL | D- VL- | EIVLTQSPPTLSLSPGERVTLSCSVSSSVSYMHWYQQK |
| | | H.21 | PGQAPKLLIYDTSKLASSIPARFSGSGSGTDYTLTISSL |
| | | | QPEDFAVYYCQQWSSNPQYTFGQGTKLEIK |
| SEQ ID NO: 3206 | VL | D- VL- | DIQLTQSPSSLSASVGDRVTITCSVSSSVSYMHWYQQK |
| | | H.22 | PGKAPKSLIYDTSKLASGVPSRFSGSGSGTDYTLTISSL |
| | | | QPEDFATYYCQQWSSNPQYTFGQGTKLEIK |
| SEQ ID NO: 3207 | VL | D- VL- | DIQLTQSPSSLSASVGDRVTITCSVSSSVSYMHWYQQK |
| | | H.23 | PGKAPKRLIYDTSKLASGVPSRFSGSGSGTEYTLTISNL |
| | | | QPEDFATYYCQQWSSNPQYTFGQGTKLEIK |
| SEQ ID NO: 3208 | VL | D- VL- | DIQLTQSPSAMSASVGDRVTITCSVSSSVSYMHWYQQ |
| | | H.24 | KPGKVPKRLIYDTSKLASGVPSRFSGSGSGTEYTLTISS |
| | | | LQPEDFATYYCQQWSSNPQYTFGQGTKLEIK |
| SEQ ID NO: 3209 | VL | D- VL- | EIVLTQSPATLSLSPGERATLSCSVSSSVSYMHWYQQK |
| | | H.25 | PGQAPKLLIYDTSKLASGIPDRFSGSGSGTDYTLTISRL |
| | | | EPEDFAVYYCQQWSSNPQYTFGQGTKLEIK |
| SEQ ID NO: 3210 | VL | D- VL- | EIVLTQSPATLSLSPGERATLSCSVSSSVSYMHWYQQK |
| | | H.26 | PGLAPKLLIYDTSKLASGIPDRFSGSGSGTDYTLTISRL |
| | | | EPEDFAVYYCQQWSSNPQYTFGQGTKLEIK |
| SEQ ID NO: 3211 | VL | D- VL- | EIVLTQSPGTLSLSPGERATLSCSVSSSVSYMHWYQQK |
| | | H.27 | PGQAPKLLIYDTSKLASGIPDRFSGSGSGTDYTLTISRL |
| | | | EPEDFAVYYCQQWSSNPQYTFGQGTKLEIK |
| SEQ ID NO: 3212 | VL | D- VL- | DIQLTQSPSSLSASVGDRVTITCSVSSSVSYMHWYQQK |
| | | H.28 | PGKAPKSLIYDTSKLASGVPSKFSGSGSGTDYTLTISSL |
| | | | QPEDFATYYCQQWSSNPQYTFGQGTKLEIK |
| SEQ ID NO: 3213 | VL | D- VL- | DIQLTQSPSSLSASVGDRVTITCSVSSSVSYMHWYQQK |
| | | H.29 | PEKAPKSLIYDTSKLASGVPSRFSGSGSGTDYTLTISSL |
| | | | QPEDFATYYCQQWSSNPQYTFGQGTKLEIK |
| SEQ ID NO: 3214 | VL | D- VL- | DIVLTQSPDSLAVSLGERATINCSVSSSVSYMHWYQQ |
| | | H.30 | KPGQPPKLLIYDTSKLASGVPDRFSGSGSGTDYTLTISS |
| | | | LQAEDVAVYYCQQWSSNPQYTFGQGTKLEIK |
| SEQ ID NO: 3215 | VL | D- VL- | EIVLTQTPLSLSITPGEQASMSCSVSSSVSYMHWYLQK |
| | | H.31 | ARPVPKLLIYDTSKLASGVPDRFSGSGSGTDYTLKISR |
| | | | VEAEDFGVYYCQQWSSNPQYTFGQGTKLEIK |
| SEQ ID NO: 3216 | VL | D- VL- | EIVLTQTPLSLSITPGEQASISCSVSSSVSYMHWYLQKA |
| | | H.32 | RPVPKLLIYDTSKLASGVPDRFSGSGSGTDYTLKISRV |
| | | | EAEDFGVYYCQQWSSNPQYTFGQGTKLEIK |
| SEQ ID NO: 3217 | VL | D- VL- | DIVLTQSPLSLPVTPGEPASISCSVSSSVSYMHWYLQK |
| | | H.33 | PGQSPKLLIYDTSKLASGVPDRFSGSGSGTDYTLKISR |
| | | | VEAEDVGVYYCQQWSSNPQYTFGQGTKLEIK |
| SEQ ID NO: 3218 | VL | D- VL- | DIVLTQSPLSLPVTLGQPASISCSVSSSVSYMHWYQQR |
| | | H.34 | PGQSPKRLIYDTSKLASGVPDRFSGSGSGTDYTLKISR |
| | | | VEAEDVGVYYCQQWSSNPQYTFGQGTKLEIK |
| SEQ ID NO: 3219 | VL | D- VL- | DIVLTQTPLSLPVTPGEPASISCSVSSSVSYMHWYLQK |
| | | H.35 | PGQSPKLLIYDTSKLASGVPDRFSGSGSGTDYTLKISR |
| | | | VEAEDVGVYYCQQWSSNPQYTFGQGTKLEIK |
| SEQ ID NO: 3220 | VL | D- VL- | DIVLTQTPLSLSVTPGQPASISCSVSSSVSYMHWYLQK |
| | | H.36 | PGQSPKLLIYDTSKLASGVPDRFSGSGSGTDYTLKISR |
| | | | VEAEDVGVYYCQQWSSNPQYTFGQGTKLEIK |
| SEQ ID NO: 3221 | VL | D- VL- | DIVLTQTPLSLSVTPGQPASISCSVSSSVSYMHWYLQK |
| | | H.37 | PGQPPKLLIYDTSKLASGVPDRFSGSGSGTDYTLKISR |
| | | | VEAEDVGVYYCQQWSSNPQYTFGQGTKLEIK |
| SEQ ID NO: 3222 | VL | D- VL- | DIQLIQSPSFLSASVGDRVSIICSVSSSVSYMHWYLQKP |
| | | H.38 | GKSPKLFIYDTSKLASGVSSRFSGRGSGTDYTLTIISLK |
| | | | PEDFAAYYCQQWSSNPQYTFGQGTKLEIK |
| SEQ ID NO: 3223 | VL | D- VL- | DIVLTQTPLSSPVTLGQPASISCSVSSSVSYMHWYQQR |
| | | H.39 | PGQPPKLLIYDTSKLASGVPDRFSGSGAGTDYTLKISR |
| | | | VEAEDVGVYYCQQWSSNPQYTFGQGTKLEIK |
| SEQ ID NO: 3224 | VL | D- VL- | EITLTQSPAFMSATPGDKVNISCSVSSSVSYMHWYQQ |
| | | H.40 | KPGEAPKFIIYDTSKLASGIPPRFSGSGYGTDYTLTINNI |
| | | | ESEDAAYYYCQQWSSNPQYTFGQGTKLEIK |

Variable HEAVY chain (VH)

| | | | |
|---|---|---|---|
| SEQ ID NO: 3225 | VH | D-VH- | EVQLVESGGGLVKPGGSLRLSCAVSGFTFRSYGMSW |
| | | H.1 | VRQAPGKGLEWVALISSGGSYTYYTDSVKGRFTISRD |
| | | | NSKNTLYLQMNSLKTEDTAVYYCSRHGGNFFDYWG |
| | | | QGTTVTVSS |
| SEQ ID NO: 3226 | VH | D- VH- | EVQLVESGGALVKPGGSLRLSCAVSGFTFRSYGMSW |
| | | H.2 | VRQAPGKGLEWVALISSGGSYTYYTDSVKGRFTISRD |
| | | | NSKNTLYLQMNSLKTEDTAVYYCSRHGGNFFDYWG |
| | | | QGTTVTVSS |
| SEQ ID NO: 3227 | VH | D- VH- | EVQLVESGGGLVQPGGSLRLSCAVSGFTFRSYGMSW |
| | | H.3 | VRQAPGKGLEWVALISSGGSYTYYTDSVKGRFTISRD |
| | | | NAKNTLYLQMNSLRAEDTAVYYCSRHGGNFFDYWG |
| | | | QGTTVTVSS |
| SEQ ID NO: 3228 | VH | D- VH- | EVQLVESGGGLVQPGGSLRLSCAVSGFTFRSYGMSW |
| | | H.4 | VRQAPGKGLEWVALISSGGSYTYYTDSVKGRFTISRD |
| | | | NAKNSLYLQMNSLRAEDTAVYYCSRHGGNFFDYWG |
| | | | QGTTVTVSS |
| SEQ ID NO: 3229 | VH | D- VH- | EVQLVESGGGLVQPGGSLRLSCAVSGFTFRSYGMSW |
| | | H.5 | VRQAPGKGLEWVALISSGGSYTYYTDSVKGRFTISRD |
| | | | NSKNSLYLQMNSLKTEDTAVYYCSRHGGNFFDYWG |
| | | | QGTTVTVSS |
| SEQ ID NO: 3230 | VH | D- VH- | EVQLVESGGGLVQPGGSLRLSCAVSGFTFRSYGMSW |
| | | H.6 | VRQAPGKGLEWVALISSGGSYTYYTDSVKGRFTISRD |
| | | | NAKNSLYLQMNSLRAEDMAVYYCSRHGGNFFDYWG |
| | | | QGTTVTVSS |
| SEQ ID NO: 3231 | VH | D- VH- | EVQLVESGGGLVQPGGSLRLSCAVSGFTFRSYGMSW |
| | | H.7 | VRQAPGKGLEWVALISSGGSYTYYTDSVKGQFTISRD |
| | | | NAKNTLYLQMNSLRAEDMAVYYCSRHGGNFFDYWG |
| | | | QGTTVTVSS |
| SEQ ID NO: 3232 | VH | D- VH- | EVQLVESGGGLVKPGRSLRLSCTVSGFTFRSYGMSWV |
| | | H.8 | RQAPGKGLEWVALISSGGSYTYYTDSVKGRFTISRDN |
| | | | SKNILYLQMNSLKTEDTAVYYCSRHGGNFFDYWGQG |
| | | | TTVTVSS |
| SEQ ID NO: 3233 | VH | D- VH- | EVQLVESGGGLVKPGGSLRLSCAVSGFTFRSYGMSW |
| | | H.9 | VRQAPGKGLEWVALISSGGSYTYYTDSVKGRFTISRD |
| | | | NAKNSLYLQMNSLRAEDTAVYYCSRHGGNFFDYWG |
| | | | QGTTVTVSS |
| SEQ ID NO: 3234 | VH | D- VH- | EVQLVESGGGLVQPGGSLKLSCAVSGFTFRSYGMSW |
| | | H.10 | VRQASGKGLEWVALISSGGSYTYYTDSVKGRFTISRD |
| | | | NSKNTLYLQMNSLKTEDTAVYYCSRHGGNFFDYWG |
| | | | QGTTVTVSS |
| SEQ ID NO: 3235 | VH | D- VH- | QVQLVESGGGVVQPGGSLRLSCAVSGFTFRSYGMSW |
| | | H.11 | VRQAPGKGLEWVALISSGGSYTYYTDSVKGRFTISRD |
| | | | NSKNTLYLQMNSLRAEDTAVYYCSRHGGNFFDYWG |
| | | | QGTTVTVSS |
| SEQ ID NO: 3236 | VH | D- VH- | QVQLVESGGGVVQPGRSLRLSCAVSGFTFRSYGMSW |
| | | H.12 | VRQAPGKGLEWVALISSGGSYTYYTDSVKGRFTISRD |
| | | | NSKNTLYLQMSSLRAEDTAVYYCSRHGGNFFDYWGQ |
| | | | GTTVTVSS |
| SEQ ID NO: 3237 | VH | D- VH- | EVQLVESGGGLVQPGGSLRLSCPVSGFTFRSYGMSWV |
| | | H.13 | RQAPGKGLEWVALISSGGSYTYYTDSVKGRFTISRDN |
| | | | ANNSLYLQMNSLRAEDTAVYYCSRHGGNFFDYWGQ |
| | | | GTTVTVSS |
| SEQ ID NO: 3238 | VH | D- VH- | EVQLVESGGGLVQPGRSLRLSCTVSGFTFRSYGMSWV |
| | | H.14 | RQAPGKGLEWVALISSGGSYTYYTDSVKGRFTISRDN |
| | | | SKNILYLQMNSLKTEDTAVYYCSRHGGNFFDYWGQG |
| | | | TTVTVSS |
| SEQ ID NO: 3239 | VH | D- VH- | EVQLVESGGGLVQPGPSLRLSCTVSGFTFRSYGMSWV |
| | | H.15 | RQAPGKGLEWVALISSGGSYTYYTDSVKGRFTISRDN |
| | | | SKNILYLQMNSLKTEDTAVYYCSRHGGNFFDYWGQG |
| | | | TTVTVSS |
| SEQ ID NO: 3240 | VH | D- VH- | EVQLVESGGGLVQPGGSLRLSCAVSGFTFRSYGMSW |
| | | H.16 | VRQAPGKGLEWVALISSGGSYTYYTDSVKGRFTISRD |
| | | | NSKNTLYLQMNSLRAEDTAVYYCSRHGGNFFDYWG |
| | | | QGTTVTVSS |
| SEQ ID NO: 3241 | VH | D- VH- | EVQLVESGGGLVQPGGSLRLSCAVSGFTFRSYGMSW |
| | | H.17 | VRQAPGKGLEWVALISSGGSYTYYTDSVKGRFTISRD |
| | | | NAKNSLYLQMNSLRDEDTAVYYCSRHGGNFFDYWG |
| | | | QGTTVTVSS |
| SEQ ID NO: 3242 | VH | D- VH- | QVQLVESGGGLVKPGGSLRLSCAVSGFTFRSYGMSW |
| | | H.18 | VRQAPGKGLEWVALISSGGSYTYYTDSVKGRFTISRD |
| | | | NAKNSLYLQMNSLRAEDTAVYYCSRHGGNFFDYWG |
| | | | QGTTVTVSS |
| SEQ ID NO: 3243 | VH | D- VH- | QVQLVESGGGVVQPGRSLRLSCAVSGFTFRSYGMSW |
| | | H.19 | VRQAPGKGLEWVALISSGGSYTYYTDSVKGRFTISRD |
| | | | NSKNTLYLQMNSLRAEDTAVYYCSRHGGNFFDYWG |
| | | | QGTTVTVSS |
| SEQ ID NO: 3244 | VH | D- VH- | EVQLLESGGGLVQPGGSLRLSCAVSGFTFRSYGMSWV |
| | | H.20 | RQAPGKGLEWVALISSGGSYTYYTDSVKGRFTISRDN |
| | | | SKNTLYLQMNSLRAEDTAVYYCSRHGGNFFDYWGQ |
| | | | GTTVTVSS |
| SEQ ID NO: 3245 | VH | D- VH- | EVQLVESGGGLVQPGGSLRLSCAVSGFTFRSYGMSW |
| | | H.21 | VRQAPGKGLEWVALISSGGSYTYYTDSVKGRFTISRH |
| | | | NSKNTLYLQMNSLRAEDTAVYYCSRHGGNFFDYWG |
| | | | QGTTVTVSS |
| SEQ ID NO: 3246 | VH | D- VH- | EVQLVESGGGLIQPGGSLRLSCAVSGFTFRSYGMSWV |
| | | H.22 | RQPPGKGLEWVALISSGGSYTYYTDSVKGRFTISRDNS |
| | | | KNTLYLQMNSLRAEDTAVYYCSRHGGNFFDYWGQG |
| | | | TTVTVSS |
| SEQ ID NO: 3247 | VH | D- VH- | EVQLVESGGGLIQPGGSLRLSCAVSGFTFRSYGMSWV |
| | | H.23 | RQAPGKGLEWVALISSGGSYTYYTDSVKGRFTISRDN |
| | | | SKNTLYLQMNSLRAEDTAVYYCSRHGGNFFDYWGQ |
| | | | GTTVTVSS |
| SEQ ID NO: 3248 | VH | D- VH- | EVQLVESGGGLVQPGRSLRLSCAVSGFTFRSYGMSW |
| | | H.24 | VRQAPGKGLEWVALISSGGSYTYYTDSVKGRFTISRD |
| | | | NAKNSLYLQMNSLRAEDTALYYCSRHGGNFFDYWG |
| | | | QGTTVTVSS |
| SEQ ID NO: 3249 | VH | D- VH- | QVQLVESGGGVVQPGRSLRLSCAVSGFTFRSYGMSW |
| | | H.25 | VRQAPGKGLEWVALISSGGSYTYYTDSVKGRFTISRD |
| | | | NSKNRLYLQMNSLRAEDTAVYYCSRHGGNFFDYWG |
| | | | QGTTVTVSS |
| SEQ ID NO: 3250 | VH | D- VH- | QVQLVESGGGVVQPGRSLRLSCAVSGFTFRSYGMSW |
| | | H.26 | VRQAPGKGLEWVALISSGGSYTYYTDSVKGRFTISRD |
| | | | NSKNTLYLQMNSLRAEGTAVYYCSRHGGNFFDYWG |
| | | | QGTTVTVSS |
| SEQ ID NO: 3251 | VH | D- VH- | QVQLVESGGGVVQPGRSLRLSCAVSGFTFRSYGMSW |
| | | H.27 | VRQAPGKGLEWVALISSGGSYTYYTDSVKGRFAISRD |
| | | | NSKNTLYLQMNSLRAEDTAVYYCSRHGGNFFDYWG |
| | | | QGTTVTVSS |
| SEQ ID NO: 3252 | VH | D- VH- | QVQLVDSGGGVVQPGRSLRLSCAVSGFTFRSYGMSW |
| | | H.28 | VRQAPGKGLEWVALISSGGSYTYYTDSVKGRFTISRD |
| | | | NSKNTLYLQMNSLRAEDTAVYYCSRHGGNFFDYWG |
| | | | QGTTVTVSS |
| SEQ ID NO: 3253 | VH | D- VH- | EVQLVESGGGVVRPGGSLRLSCAVSGFTFRSYGMSW |
| | | H.29 | VRQAPGKGLEWVALISSGGSYTYYTDSVKGRFTISRD |
| | | | NAKNSLYLQMNSLRAEDTALYHCSRHGGNFFDYWG |
| | | | QGTTVTVSS |
| SEQ ID NO: 3254 | VH | D- VH- | EVQLVESGGVVVQPGGSLRLSCAVSGFTFRSYGMSW |
| | | H.30 | VRQAPGKGLEWVALISSGGSYTYYTDSVKGRFTISRD |
| | | | NSKNSLYLQMNSLRAEDTALYYCSRHGGNFFDYWGQ |
| | | | GTTVTVSS |
| SEQ ID NO: 3255 | VH | D- VH- | EVQLVESGGGVVQPGGSLRLSCAVSGFTFRSYGMSW |
| | | H.31 | VRQAPGKGLEWVALISSGGSYTYYTDSVKGRFTISRD |
| | | | NSKNSLYLQMNSLRTEDTALYYCSRHGGNFFDYWGQ |
| | | | GTTVTVSS |
| SEQ ID NO: 3256 | VH | D- VH- | EVQLVESGGVVVQPGGSLRLSCAVSGFTFRSYGMSW |
| | | H.32 | VRQAPGKGLEWVALISSGGSYTYYTDSVKGRFTISRD |
| | | | NSKNSLYLQMNSLRTEDTALYYCSRHGGNFFDYWGQ |
| | | | GTTVTVSS |
| SEQ ID NO: 3257 | VH | D- VH- | EVQLVETGGGLIQPGGSLRLSCAVSGFTFRSYGMSWV |
| | | H.33 | RQAPGKGLEWVALISSGGSYTYYTDSVKGRFTISRDN |
| | | | SKNTLYLQMNSLRAEDTAVYYCSRHGGNFFDYWGQ |
| | | | GTTVTVSS |
| SEQ ID NO: 3258 | VH | D- VH- | EVQLVESGGGLVQPGGSLRLSCAVSGFTFRSYGMSW |
| | | H.34 | VRQATGKGLEWVALISSGGSYTYYTDSVKGRFTISRE |
| | | | NAKNSLYLQMNSLRAGDTAVYYCSRHGGNFFDYWG |
| | | | QGTTVTVSS |
| SEQ ID NO: 3259 | VH | D- VH- | EVQLVESRGVLVQPGGSLRLSCAVSGFTFRSYGMSW |
| | | H.35 | VRQAPGKGLEWVALISSGGSYTYYTDSVKGRFTISRD |
| | | | NSKNTLHLQMNSLRAEDTAVYYCSRHGGNFFDYWG |
| | | | QGTTVTVSS |
| SEQ ID NO: 3260 | VH | D- VH- | EVQLVESGGGLVQPGRSLRLSCAVSGFTFRSYGMSW |
| | | H.36 | VRQAPGKGLEWVALISSGGSYTYYTDSVKGRFTISRD |
| | | | NAKNSLYLQMNSLRAEDMALYYCSRHGGNFFDYWG |
| | | | QGTTVTVSS |
| SEQ ID NO: 3261 | VH | D- VH- | QVQLVESGGGLVQPGGSLRLSCSVSGFTFRSYGMSW |
| | | H.37 | VRQAPGKGLEWVALISSGGSYTYYTDSVKGRFTISRD |
| | | | NSKNTLYLQMNSLRAEDTAVYYCSRHGGNFFDYWG |
| | | | QGTTVTVSS |
| SEQ ID NO: 3262 | VH | D- VH- | EVQLVESGGGLVQPGGSLRLSCSVSGFTFRSYGMSWV |
| | | H.38 | RQAPGKGLEWVALISSGGSYTYYTDSVKGRFTISRDN |
| | | | SKNTLYLQMSSLRAEDTAVYYCSRHGGNFFDYWGQG |
| | | | TTVTVSS |
| SEQ ID NO: 3263 | VH | D- VH- | QVQLVESGGGVVQPGRSLRLSCAVSGFTFRSYGMSW |
| | | H.39 | VRQAPGKGLEWVALISSGGSYTYYTDSVKGRFTISRD |
| | | | NSTNTLFLQMNSLRAEDTAVYYCSRHGGNFFDYWGQ |
| | | | GTTVTVSS |
| SEQ ID NO: 3264 | VH | D- VH- | QVQLLESGGGLVKPGGSLRLSCAVSGFTFRSYGMSW |
| | | H.40 | VRQAPGKGLEWVALISSGGSYTYYTDSVKGRFTISRD |
| | | | NAKNSLYLQMNSLRAEDTAVYYCSRHGGNFFDYWG |
| | | | QGTTVTVSS |
| SEQ ID NO: 3265 | VH | D- VH- | EVQLVESGEGLVQPGGSLRLSCAVSGFTFRSYGMSWV |
| | | H.41 | RQAPGKGLEWVALISSGGSYTYYTDSVKGRFTISRDN |
| | | | SKNTLYLQMGSLRAEDMAVYYCSRHGGNFFDYWGQ |
| | | | GTTVTVSS |
| SEQ ID NO: 3266 | VH | D- VH- | EVQLVESGGGLVQPGGSLRLSCAVSGFTFRSYGMSW |
| | | H.42 | VRQAPGKGLEWVALISSGGSYTYYTDSVKGRFTISRD |
| | | | NSKNTLYLQMGSLRAEDMAVYYCSRHGGNFFDYWG |
| | | | QGTTVTVSS |
| SEQ ID NO: 3267 | VH | D- VH- | EVQLVESGGGLVQPGGSLRLSCSVSGFTFRSYGMSWV |
| | | H.43 | RQAPGKGLEWVALISSGGSYTYYTDSVKGRFTISRDN |
| | | | SKNTLYVQMSSLRAEDTAVYYCSRHGGNFFDYWGQ |
| | | | GTTVTVSS |
| SEQ ID NO: 3268 | VH | D- VH- | EVQLVESGGGLVQPGGSLRLSCAVSGFTFRSYGMSW |
| | | H.44 | VRQAPGKGLEWVALISSGGSYTYYTDSVKGRFIISRD |
| | | | NSRNSLYLQKNRRRAEDMAVYYCSRHGGNFFDYWG |
| | | | QGTTVTVSS |
| SEQ ID NO: 3269 | VH | D- VH- | EVQLVESGGGLVQPGGSLRLSCAVSGFTFRSYGMSW |
| | | H.45 | VHQAPGKGLEWVALISSGGSYTYYTDSVKGRFIISRD |
| | | | NSRNTLYLQTNSLRAEDTAVYYCSRHGGNFFDYWGQ |
| | | | GTTVTVSS |
| SEQ ID NO: 3270 | VH | D- VH- | EVHLVESGGGLVQPGGALRLSCAVSGFTFRSYGMSW |
| | | H.46 | VRQATGKGLEWVALISSGGSYTYYTDSVKGRFTISRE |
| | | | NAKNSLYLQMNSLRAGDTAVYYCSRHGGNFFDYWG |
| | | | QGTTVTVSS |
| SEQ ID NO: 3271 | VH | D- VH- | EVQLVESGGGLVQPRGSLRLSCAVSGFTFRSYGMSW |
| | | H.47 | VRQAPGKGLEWVALISSGGSYTYYTDSVKGRFTISRD |
| | | | NSKNTLYLQMNNLRAEGTAVYYCSRHGGNFFDYWG |
| | | | QGTTVTVSS |
| SEQ ID NO: 3272 | VH | D- VH- | EVQLVESGGGLVQPRGSLRLSCAVSGFTFRSYGMSW |
| | | H.48 | VRQAPGKGLEWVALISSGGSYTYYTDSVKGRFTISRD |
| | | | NSKNTLYLQMNNLRAEGTAAYYCSRHGGNFFDYWG |
| | | | QGTTVTVSS |
| SEQ ID NO: 3273 | VH | D- VH- | QVQLVQSGAEVKKPGASVKVSCKVSGFTFRSYGMSW |
| | | H.49 | VRQAPGKGLEWVALISSGGSYTYYTDSVKGRFTITRD |
| | | | NSTNTLYMELSSLRSEDTAVYYCSRHGGNFFDYWGQ |
| | | | GTTVTVSS |
| SEQ ID NO: 3274 | VH | D- VH- | QVQLVQSGSELKKPGASVKVSCKVSGFTFRSYGMSW |
| | | H.50 | VRQAPGQGLEWVALISSGGSYTYYTDSVKGRFVISRD |
| | | | NSVNTLYLQISSLKAEDTAVYYCSRHGGNFFDYWGQ |
| | | | GTTVTVSS |

In some embodiments, the anti-TCRβ V10 antibody molecule comprises a VH or a VL of an antibody described in Table 12, or a sequence with at least 80%, 85%, 90%, 95%, 96%, 97%, 98%, 99% or more identity thereto.

In some embodiments, the anti-TCRβ V10 antibody molecule comprises a VH and a VL of an antibody described in Table 12, or a sequence with at least 80%, 85%, 90%, 95%, 96%, 97%, 98%, 99% or more identity thereto.

### Additional Anti-TCRVβ Antibodies

Additional exemplary anti-TCRβV antibodies of the disclosure are provided in Table 13. In some embodiments, the anti-TCRβV antibody is a humanized antibody, e.g., as provided in Table 13. In some embodiments, the anti-TCRβV antibody comprises one or more (e.g., all three) of a LC CDR1, LC CDR2, and LC CDR3 provided in Table 13; and/or one or more (e.g., all three) of a HC CDR1, HC CDR2, and HC CDR3 provided in Table 13, or a sequence with at least 95% identity thereto. In some embodiments, the anti-TCRβV antibody comprises a variable heavy chain (VH) and/or a variable light chain (VL) provided in Table 13, or a sequence with at least 95% identity thereto.

TABLE 13
Amino acid sequences for additional anti-TCRβ V antibodies
Amino acid and nucleotide sequences for murine and humanized antibody molecules which bind to
various TCRVB families are disclosed. The amino acid the heavy and light chain CDRs, and the amino
acid and nucleotide sequences of the heavy and light chain variable regions, and the heavy and light
chains are shown. Antibodies disclosed in the table include, MPB2D5, CAS1.1.3, IMMU222, REA1062,
JOVI-3, IMMU546 and MR5-2. MPB2D5 binds human TCRβV 20-1 (TCRβV2 per old nomenclature).
CAS1.1.3 binds human TCRβV 27 (TCRβV14 per old nomenclature). IMMU 222 binds human TCRβV
6-5, TCRβV 6-6, or TCRβV 6-9 (TCRβV13.1 per old nomenclature). REA1062 binds human TCRβV 5-
1). JOVI-3 binds human TCRβV 28 (TCRβV3.1 per old nomenclature). IMMU546 binds human TCRβV
2. MR5-2 binds human TCRVβ 13-2.
Binds to human TCRVB 20-1
MPB2D5 (murine), also referred to here as BJ1188, BJ1190 and REA654; or Antibody G
Binds to human TCRVβ 20-1

| | | |
|---|---|---|
| SEQ ID NO: 1102 | HC CDR1 (Kabat) | SAYMH |
| SEQ ID NO: 1103 | HC CDR2 (Kabat) | RIDPATGKTKYAPKFQA |
| SEQ ID NO: 1104 | HC CDR3 (Kabat) | SLNWDYGLDY |
| SEQ ID NO: 1105 | HC CDR1 (Chothia) | GFNIKSA |
| SEQ ID NO: 1106 | HC CDR2 (Chothia) | DPATGK |
| SEQ ID NO: 1104 | HC CDR3 (Chothia) | SLNWDYGLDY |
| SEQ ID NO: 1107 | HC CDR1 (Combined) | GFNIKSAYMH |
| SEQ ID NO: 1103 | HC CDR2 (Combined) | RIDPATGKTKYAPKFQA |
| SEQ ID NO: 1104 | HC CDR3 (Combined) | SLNWDYGLDY |
| SEQ ID NO: 7489 | LC CDR1 (Kabat) | RASKSVSILGTHLIH |
| SEQ ID NO: 1108 | LC CDR2 (Kabat) | AASNLES |
| SEQ ID NO: 1109 | LC CDR3 (Kabat) | QQSIEDPWT |
| SEQ ID NO: 1110 | LC CDR1 (Chothia) | SKSVSILGTHL |
| SEQ ID NO: 1108 | LC CDR2 (Chothia) | AASNLES |
| SEQ ID NO: 1109 | LC CDR3 (Chothia) | QQSIEDPWT |
| SEQ ID NO: 7489 | LC CDR1 (Combined) | RASKSVSILGTHLIH |
| SEQ ID NO: 1108 | LC CDR2 (Combined) | AASNLES |
| SEQ ID NO: 1109 | LC CDR3(Combined) | QQSIEDPWT |
| SEQ ID NO: 1111 | VL | DIVLTQSPASLAVSLGQRATISCRASKSVSILGTH |
| | | LIHWYQQKPGQPPKLLIYAASNLESGVPARFSGS |
| | | GSETVFTLNIHPVEEEDAATYFCQQSIEDPWTFG |
| | | GGTKLGIK |
| SEQ ID NO: 1112 | VH | EVQLQQSVADLVRPGASLKLSCTASGFNIKSAY |
| | | MHWVIQRPDQGPECLGRIDPATGKTKYAPKFQA |
| | | KATITADTSSNTAYLQLSSLTSEDTAIYYCTRSLN |
| | | WDYGLDYWGQGTSVTVSS |

VH for MPB2D5 (humanized) also referred to as Antibody G-H (humanized)
Binds to human TCRVβ 20-1

| | | |
|---|---|---|
| SEQ ID NO: 1113 | VH - 1 | QVQLVQSGAEVKKPGASVKVSCKASGFNIKSAY |
| | | MHWVRQAPGQGLEWMGRIDPATGKTKYAPKF |
| | | QARVTMTADTSTNTAYMELSSLRSEDTAVYYC |
| | | ARSLNWDYGLDYWGQGTLVTVSS |
| SEQ ID NO: 1114 | VH - 2 | QVQLVQSGAEVKKPGASVKVSCKASGFNIKSAY |
| | | MHWVRQAPGQEPGCMGRIDPATGKTKYAPKFQ |
| | | ARVTMTADTSINTAYTELSSLRSEDTATYYCARS |
| | | LNWDYGLDYWGQGTLVTVSS |
| SEQ ID NO: 1115 | VH - 3 | QVQLVQSGAEVKKPGSSVKVSCKASGFNIKSAY |
| | | MHWVRQAPGQGLEWMGRIDPATGKTKYAPKF |
| | | QARVTITADTSTNTAYMELSSLRSEDTAVYYCA |
| | | RSLNWDYGLDYWGQGTLVTVSS |
| SEQ ID NO: 1116 | VH - 4 | QVQLVQSGAEVKKPGASVKVSCKASGFNIKSAY |
| | | MHWVRQAPGQRLEWMGRIDPATGKTKYAPKF |
| | | QARVTITADTSANTAYMELSSLRSEDTAVYYCA |
| | | RSLNWDYGLDYWGQGTLVTVSS |

VL for MPB2D5 (humanized) also referred to as Antibody G-H (humanized)
Binds to human TCRVβ 20-1

| | | |
|---|---|---|
| SEQ ID NO: 1117 | VL - 1 | EIVLTQSPATLSLSPGERATLSCRASKSVSILGTH |
| | | LIHWYQQKPGQAPRLLIYAASNLESGIPARFSGS |
| | | GSETDFTLTISSLEPEDFAVYFCQQSIEDPFGGGT |
| | | KVEIK |
| SEQ ID NO: 1118 | VL - 2 | EIVLTQSPATLSLSPGERATLSCRASKSVSILGTH |
| | | LIHWYQQKPGLAPRLLIYAASNLESGIPDRESGS |
| | | GSETDFTLTISRLEPEDFAVYFCQQSIEDPFGGGT |
| | | KVEIK |
| SEQ ID NO: 1119 | VL - 3 | EIVLTQSPGTLSLSPGERATLSCRASKSVSILGTH |
| | | LIHWYQQKPGQAPRLLIYAASNLESGIPDRESGS |
| | | GSETDFTLTISRLEPEDFAVYFCQQSIEDPFGGGT |
| | | KVEIK |

CAS1.1.3 (murine) also referred to herein as BJ1460; or Antibody H
Binds to human TCRVβ 27

| | | |
|---|---|---|
| SEQ ID NO: 1120 | HC CDR1 (Kabat) | DTYMY |
| SEQ ID NO: 1121 | HC CDR2 (Kabat) | RIDPANGNTKYDPKFQD |
| SEQ ID NO: 1122 | HC CDR3 (Kabat) | GSYYYAMDY |
| SEQ ID NO: 1123 | HC CDR1 (Chothia) | GFKTEDT |
| SEQ ID NO: 1124 | HC CDR2 (Chothia) | DPANGN |
| SEQ ID NO: 1122 | HC CDR3 (Chothia) | GSYYYAMDY |
| SEQ ID NO: 1125 | HC CDR1 (Combined) | GFKTEDTYMY |
| SEQ ID NO: 1121 | HC CDR2 (Combined) | RIDPANGNTKYDPKFQD |
| SEQ ID NO: 1122 | HC CDR3(Combined) | GSYYYAMDY |
| SEQ ID NO: 1126 | LC CDR1 (Kabat) | RASESVDSYGNSFMH |
| SEQ ID NO: 1127 | LC CDR2 (Kabat) | RASNLES |
| SEQ ID NO: 1128 | LC CDR3 (Kabat) | QQSNEDPYT |
| SEQ ID NO: 1129 | LC CDR1 (Chothia) | SESVDSYGNSF |
| SEQ ID NO: 1127 | LC CDR2 (Chothia) | RASNLES |
| SEQ ID NO: 1128 | LC CDR3 (Chothia) | QQSNEDPYT |
| SEQ ID NO: 1126 | LC CDR1 (Combined) | RASESVDSYGNSFMH |
| SEQ ID NO: 1127 | LC CDR2 (Combined) | RASNLES |
| SEQ ID NO: 1128 | LC CDR3(Combined) | QQSNEDPYT |
| SEQ ID NO: 7490 | VL | DIVLTQSPASLAVSLGQRATISCRASESVDSYGN |
| | | SFMHWYQQKPGQPPKLLIYRASNLESGIPARFSG |
| | | SGSRTDFTLTINPVEADDVATYYCQQSNEDPYTF |
| | | GGGTKLEIK |
| SEQ ID NO: 1130 | VH | EVQLQQSGAELVKPGASVKLSCTASGFKTEDTY |
| | | MYWVKQRPEQGLEWIGRIDPANGNTKYDPKFQ |
| | | DKATITADSSSNTAYLQLSSLPSEDTAVYYCARG |
| | | SYYYAMDYWGQGTSVTVSS |

VH for CAS1.1.3 (humanized) also referred to as Antibody H-H (humanized)
Binds to human TCRVβ 27

| | | |
|---|---|---|
| SEQ ID NO: 1131 | VH - 1 | QVQLVQSGAEVKKPGSSVKVSCKASGFKTEDTY |
| | | MYWVRQAPGQGLEWIGRIDPANGNTKYDPKFQ |
| | | DRATITADSSTNTAYMELSSLRSEDTAVYYCAR |
| | | GSYYYAMDYWGQGTLVTVSS |
| SEQ ID NO: 1132 | VH - 2 | QVQLVQSGAEVKKPGASVKVSCKASGFKTEDT |
| | | YMYWVRQAPGQRLEWIGRIDPANGNTKYDPKF |
| | | QDRATITADSSANTAYMELSSLRSEDTAVYYCA |
| | | RGSYYYAMDYWGQGTLVTVSS |
| SEQ ID NO: 1133 | VH - 3 | EVQLVESGGGLVQPGGSLKLSCAASGFKTEDTY |
| | | MYWVRQASGKGLEWIGRIDPANGNTKYDPKFQ |
| | | DRATISADSSKNTAYLQMNSLKTEDTAVYYCAR |
| | | GSYYYAMDYWGQGTLVTVSS |
| SEQ ID NO: 1134 | VH - 4 | EVQLVQSGAEVKKPGESLRISCKASGFKTEDTY |
| | | MYWVRQMPGKGLEWIGRIDPANGNTKYDPKFQ |
| | | DQATISADSSINTAYLQWSSLKASDTAMYYCAR |
| | | GSYYYAMDYWGQGTLVTVSS |
| SEQ ID NO: 1135 | VH - 5 | QVQLVQSGSELKKPGASVKVSCKASGFKTEDTY |
| | | MYWVRQAPGQGLEWIGRIDPANGNTKYDPKFQ |
| | | DRAVISADSSVNTAYLQISSLKAEDTAVYYCAR |
| | | GSYYYAMDYWGQGTLVTVSS |

VL for CAS1.1.3 (humanized) also referred to as Antibody H-H (humanized)
Binds to human TCRVβ 27

| | | |
|---|---|---|
| SEQ ID NO: 1136 | VL - 1 | DIVLTQSPDSLAVSLGERATINCRASESVDSYGN |
| | | SFMHWYQQKPGQPPKLLIYRASNLESGVPDRFS |
| | | GSGSRTDFTLTISSLQAEDVAVYYCQQSNEDPYT |
| | | FGQGTKLEIK |
| SEQ ID NO: 1137 | VL - 2 | EIVLTQSPATLSLSPGERATLSCRASESVDSYGNS |
| | | FMHWYQQKPGQAPKLLIYRASNLESGIPARFSGS |
| | | GSRTDFTLTISRLEPEDFAVYYCQQSNEDPYTFG |
| | | QGTKLEIK |
| SEQ ID NO: 1138 | VL - 3 | DIQLTQSPSSLSASVGDRVTITCRASESVDSYGNS |
| | | FMHWYQQKPGQAPKLLIYRASNLESGVPSRFSG |
| | | SGSRTDFTLTISSLQPEDVATYYCQQSNEDPYTF |
| | | GQGTKLEIK |
| SEQ ID NO: 1139 | VL - 4 | AIQLTQSPSSLSASVGDRVTITCRASESVDSYGNS |
| | | FMHWYQQKPGKAPKLLIYRASNLESGVPSRFSG |
| | | SGSRTDFTLTISSLQPEDFATYYCQQSNEDPYTFG |
| | | QGTKLEIK |
| SEQ ID NO: 1140 | VL - 5 | EIVLTQSPDFQSVTPKEKVTITCRASESVDSYGNS |
| | | FMHWYQQKPDQSPKLLIYRASNLESGVPSRFSG |
| | | SGSRTDFTLTINSLEAEDAATYYCQQSNEDPYTF |
| | | GQGTKLEIK |

IMMU222 (murine) also referred to as BJ1461; or Antibody I
Binds to human TCRVβ 6-5,6-6,6-9

| | | |
|---|---|---|
| SEQ ID NO: 1141 | HC CDR1 (Kabat) | SYAMS |
| SEQ ID NO: 1142 | HC CDR2 (Kabat) | HISNGGDYIYYADTVKG |
| SEQ ID NO: 1143 | HC CDR3 (Kabat) | PSYYSDPWFFDV |
| SEQ ID NO: 1144 | HC CDR1 (Chothia) | GFTFRSY |
| SEQ ID NO: 1145 | HC CDR2 (Chothia) | SNGGDY |
| SEQ ID NO: 1143 | HC CDR3 (Chothia) | PSYYSDPWFFDV |
| SEQ ID NO: 1146 | HC CDR1 (Combined) | GFTFRSYAMS |
| SEQ ID NO: 1142 | HC CDR2 (Combined) | HISNGGDYIYYADTVKG |
| SEQ ID NO: 1143 | HC CDR3(Combined) | PSYYSDPWFFDV |
| SEQ ID NO: 1147 | LC CDR1 (Kabat) | SAGSSVSFMH |
| SEQ ID NO: 1148 | LC CDR2 (Kabat) | DTSKLAS |
| SEQ ID NO: 1149 | LC CDR3 (Kabat) | LOGSGFPLT |
| SEQ ID NO: 1150 | LC CDR1 (Chothia) | GSSVSF |
| SEQ ID NO: 1148 | LC CDR2 (Chothia) | DTSKLAS |
| SEQ ID NO: 1149 | LC CDR3 (Chothia) | LQGSGFPLT |
| SEQ ID NO: 1147 | LC CDR1 (Combined) | SAGSSVSFMH |
| SEQ ID NO: 1148 | LC CDR2 (Combined) | DTSKLAS |
| SEQ ID NO: 1149 | LC CDR3(Combined) | LQGSGFPLT |
| SEQ ID NO: 1151 | VL | ENVLTQSPAIMSASPGEKVTMTCSAGSSVSFMH |
| | | WYQQKSSTSPKLWIYDTSKLASGVPGRFSGSGS |
| | | GNSFSLTISSMEAEDVAIYYCLQGSGFPLTFGSGT |
| | | KLEIK |
| SEQ ID NO: 1152 | VH | DVKLVESGEGLVKPGGSLKLSCAASGFTFRSYA |
| | | MSWVRQTPEKRLEWVAHISNGGDYIYYADTVK |
| | | GRFTISRDNARNTLYLQMSSLKSEDTAMYYCTR |
| | | PSYYSDPWFFDVWGTGTTVTVSS |

VH for IMMU222 (humanized) also referred to as Antibody I-H
Binds to human TCRVβ 6-5,6-6,6-9

| | | |
|---|---|---|
| SEQ ID NO: 1153 | VH - 1 | EVQLVESGGGLVQPGGSLRLSCAASGFTFRSYA |
| | | MSWVRQAPGKGLEWVAHISNGGDYIYYADTVK |
| | | GRFTISRDNAKNSLYLQMNSLRAEDTAVYYCTR |
| | | PSYYSDPWFFDVWGQGTTVTVSS |
| SEQ ID NO: 1154 | VH - 2 | QVQLVESGGGVVQPGRSLRLSCAASGFTFRSYA |
| | | MSWVRQAPGKGLEWVAHISNGGDYIYYADTVK |
| | | GRFTISRDNSKNTLYLQMSSLRAEDTAVYYCTR |
| | | PSYYSDPWFFDVWGQGTTVTVSS |
| SEQ ID NO: 1155 | VH - 3 | EVQLVESGGGLVQPGGSLRLSCAASGFTFRSYA |
| | | MSWVRQAPGKGLEWVAHISNGGDYIYYADTVK |
| | | GRFTISRDNSKNTLYLQMNSLRAEDTAVYYCTR |
| | | PSYYSDPWFFDVWGQGTTVTVSS |
| SEQ ID NO: 1156 | VH - 4 | QVQLVQSGSELKKPGASVKVSCKASGFTFRSYA |
| | | MSWVRQAPGQGLEWVAHISNGGDYIYYADTVK |
| | | GRFVISRDNSVNTLYLQISSLKAEDTAVYYCTRP |
| | | SYYSDPWFFDVWGQGTTVTVSS |
| SEQ ID NO: 1157 | VH - 5 | QVQLVQSGAEVKKPGASVKVSCKASGFTFRSYA |
| | | MSWVRQAPGQRLEWVAHISNGGDYIYYADTVK |
| | | GRFTITRDNSANTLYMELSSLRSEDTAVYYCTRP |
| | | SYYSDPWFFDVWGQGTTVTVSS |

VL for IMMU222 (humanized)) also referred to as Antibody I-H
Binds to human TCRVβ 6-5,6-6,6-9

| | | |
|---|---|---|
| SEQ ID NO: 1158 | VL - 1 | ENVLTQSPATLSLSPGERATLSCSAGSSVSFMHW |
| | | YQQKPGQAPKLLIYDTSKLASGIPARFSGSGSGN |
| | | DFTLTISSLEPEDFAVYYCLQGSGFPLTFGQGTKL |
| | | EIK |
| SEQ ID NO: 1159 | VL - 2 | ENVLTQSPDFQSVTPKEKVTITCSAGSSVSFMHW |
| | | YQQKPDQSPKLLIYDTSKLASGVPSRFSGSGSGN |
| | | DFTLTINSLEAEDAATYYCLQGSGFPLTFGQGTK |
| | | LEIK |
| SEQ ID NO: 1160 | VL - 3 | DNQLTQSPSSLSASVGDRVTITCSAGSSVSFMHW |
| | | YQQKPGKVPKLLIYDTSKLASGVPSRFSGSGSGN |
| | | DFTLTISSLQPEDVATYYCLQGSGFPLTFGQGTK |
| | | LEIK |
| SEQ ID NO: 1161 | VL - 4 | ANQLTQSPSSLSASVGDRVTITCSAGSSVSFMHW |
| | | YQQKPGKAPKLLIYDTSKLASGVPSRFSGSGSGN |
| | | DFTLTISSLQPEDFATYYCLQGSGFPLTFGQGTKL |
| | | EIK |
| SEQ ID NO: 1162 | VL - 5 | DNVLTQSPDSLAVSLGERATINCSAGSSVSFMH |
| | | WYQQKPGQPPKLLIYDTSKLASGVPDRFSGSGS |
| | | GNDFTLTISSLQAEDVAVYYCLQGSGFPLTFGQG |
| | | TKLEIK |

REA1062 (murine), also referred to as BJ1189 or as Antibody J
Binds to human TCRVβ 5-1

| | | |
|---|---|---|
| SEQ ID NO: 1163 | HC CDR1 (Kabat) | DYNIH |
| SEQ ID NO: 1164 | HC CDR2 (Kabat) | YINPYNGRTGYNQKFKA |
| SEQ ID NO: 1165 | HC CDR3 (Kabat) | WDGSSYFDY |
| SEQ ID NO: 1166 | HC CDR1 (Chothia) | GYTFTDYNIH |
| SEQ ID NO: 1167 | HC CDR2 (Chothia) | NPYNGR |
| SEQ ID NO: 1165 | HC CDR3 (Chothia) | WDGSSYFDY |
| SEQ ID NO: 1166 | HC CDR1 (Combined) | GYTFTDYNIH |
| SEQ ID NO: 1164 | HC CDR2 (Combined) | YINPYNGRTGYNQKFKA |
| SEQ ID NO: 1165 | HC CDR3(Combined) | WDGSSYFDY |
| SEQ ID NO: 1168 | LC CDR1 (Kabat) | SASSSVSYMH |
| SEQ ID NO: 1169 | LC CDR2 (Kabat) | EISKLAS |
| SEQ ID NO: 1170 | LC CDR3 (Kabat) | QQWNYPLLT |
| SEQ ID NO: 1171 | LC CDR1 (Chothia) | SSSVSY |
| SEQ ID NO: 1169 | LC CDR2 (Chothia) | EISKLAS |
| SEQ ID NO: 1170 | LC CDR3 (Chothia) | QQWNYPLLT |
| SEQ ID NO: 1168 | LC CDR1 (Combined) | SASSSVSYMH |
| SEQ ID NO: 1169 | LC CDR2 (Combined) | EISKLAS |
| SEQ ID NO: 1170 | LC CDR3(Combined) | QQWNYPLLT |
| SEQ ID NO: 7491 | VL | EIVLTQSPAITAASLGQKVTITCSASSSVSYMHW |
| | | YQQKSGTSPKPWIYEISKLASGVPARFSGSGSGT |
| | | SYSLTISSMEAEDAAIYYCQQWNYPLLTFGAGT |
| | | KLELK |
| SEQ ID NO: 1172 | VH | EVQLQQSGPVLVKPGASVRMSCKASGYTFTDY |
| | | NIHWVKQSHGRSLEWVGYINPYNGRTGYNQKF |
| | | KAKATLTVDKSSSTAYMDLRSLTSEDSAVYYCA |
| | | RWDGSSYFDYWGQGTTLTVSS |

VH for REA1062 (humanized) also referred to as Antibody J-H
Binds to human TCRVβ 5-1

| | | |
|---|---|---|
| SEQ ID NO: 1173 | VH - 1 | QVQLVQSGAEVKKPGSSVKVSCKASGYTFTDY |
| | | NIHWVRQAPGQGLEWVGYINPYNGRTGYNQKF |
| | | KARATLTVDKSTSTAYMELSSLRSEDTAVYYCA |
| | | RWDGSSYFDYWGQGTTVTVSS |
| SEQ ID NO: 1174 | VH - 2 | QVQLVQSGAEVKKPGASVKVSCKASGYTFTDY |
| | | NIHWVRQAPGQGLEWVGYINPYNGRTGYNQKF |
| | | KARATLTVDKSTSTAYMELRSLRSDDMAVYYC |
| | | ARWDGSSYFDYWGQGTTVTVSS |
| SEQ ID NO: 1175 | VH - 3 | QVQLVQSGAEVKKPGASVKVSCKASGYTFTDY |
| | | NIHWVRQATGQGLEWVGYINPYNGRTGYNQKF |
| | | KARATLTVNKSISTAYMELSSLRSEDTAVYYCA |
| | | RWDGSSYFDYWGQGTTVTVSS |
| SEQ ID NO: 1176 | VH - 4 | EVQLVESGGGLVQPGRSLRLSCTASGYTFTDYNI |
| | | HWVRQAPGKGLEWVGYINPYNGRTGYNQKFK |
| | | ARATLSVDKSKSIAYLQMNSLKTEDTAVYYCAR |
| | | WDGSSYFDYWGQGTTVTVSS |
| SEQ ID NO: 1177 | VH - 5 | QVQLVQSGSELKKPGASVKVSCKASGYTFTDYN |
| | | IHWVRQAPGQGLEWVGYINPYNGRTGYNQKFK |
| | | ARAVLSVDKSVSTAYLQISSLKAEDTAVYYCAR |
| | | WDGSSYFDYWGQGTTVTVSS |

VL for REA1062 (humanized) also referred to as Antibody J-H
Binds to human TCRVβ 5-1

| | | |
|---|---|---|
| SEQ ID NO: 1178 | VL - 1 | EIVLTQSPATLSLSPGERATLSCSASSSVSYMHW |
| | | YQQKPGQAPKLLIYEISKLASGIPARFSGSGSGTD |
| | | YTLTISSLEPEDFAVYYCQQWNYPLLTFGQGTKL |
| | | EIK |
| SEQ ID NO: 1179 | VL - 2 | EIVLTQSPATLSLSPGERATLSCSASSSVSYMHW |
| | | YQQKPGQAPKLLIYEISKLASGIPARFSGSGSGTD |
| | | YTLTISRLEPEDFAVYYCQQWNYPLLTFGQGTK |
| | | LEIK |
| SEQ ID NO: 1180 | VL - 3 | EIVLTQSPDFQSVTPKEKVTITCSASSSVSYMHW |
| | | YQQKPDQSPKLLIYEISKLASGVPSRFSGSGSGTD |
| | | YTLTINSLEAEDAATYYCQQWNYPLLTFGQGTK |
| | | LEIK |
| SEQ ID NO: 1181 | VL - 4 | DIQLTQSPSFLSASVGDRVTITCSASSSVSYMHW |
| | | YQQKPGKAPKLLIYEISKLASGVPSRFSGSGSGTE |
| | | YTLTISSLQPEDFATYYCQQWNYPLLTFGQGTKL |
| | | EIK |
| SEQ ID NO: 1182 | VL - 5 | AIQLTQSPSSLSASVGDRVTITCSASSSVSYMHW |
| | | YQQKPGKAPKLLIYEISKLASGVPSRFSGSGSGT |
| | | DYTLTISSLQPEDFATYYCQQWNYPLLTFGQGT |
| | | KLEIK |
| SEQ ID NO: 1183 | VL - 6 | AIRLTQSPFSLSASVGDRVTITCSASSSVSYMHW |
| | | YQQKPAKAPKLFIYEISKLASGVPSRFSGSGSGT |
| | | DYTLTISSLQPEDFATYYCQQWNYPLLTFGQGT |
| | | KLEIK |
| SEQ ID NO: 1184 | VL - 7 | DIVLTQSPDSLAVSLGERATINCSASSSVSYMHW |
| | | YQQKPGQPPKLLIYEISKLASGVPDRFSGSGSGT |
| | | DYTLTISSLQAEDVAVYYCQQWNYPLLTFGQGT |
| | | KLEIK |

JOVI-3 (murine), also referred to as BJ1187 or Antibody K
Binds to human TCRVβ 28

| | | |
|---|---|---|
| SEQ ID NO: 1185 | HC CDR1 (Kabat) | GSWMN |
| SEQ ID NO: 1186 | HC CDR2 (Kabat) | RIYPGDGDTDYSGKFKG |
| SEQ ID NO: 1187 | HC CDR3 (Kabat) | SGYFNYVPVFDY |
| SEQ ID NO: 1188 | HC CDR1 (Chothia) | GYTFSGS |
| SEQ ID NO: 1189 | HC CDR2 (Chothia) | YPGDGD |
| SEQ ID NO: 1187 | HC CDR3 (Chothia) | SGYFNYVPVFDY |
| SEQ ID NO: 1190 | HC CDR1 (Combined) | GYTFSGSWMN |
| SEQ ID NO: 1186 | HC CDR2 (Combined) | RIYPGDGDTDYSGKFKG |
| SEQ ID NO: 1187 | HC CDR3(Combined) | SGYFNYVPVFDY |
| SEQ ID NO: 1191 | LC CDR1 (Kabat) | SANSTVGYIH |
| SEQ ID NO: 1192 | LC CDR2 (Kabat) | TTSNLAS |
| SEQ ID NO: 1193 | LC CDR3 (Kabat) | HQWSFYPT |
| SEQ ID NO: 1194 | LC CDR1 (Chothia) | NSTVGY |
| SEQ ID NO: 1192 | LC CDR2 (Chothia) | TTSNLAS |
| SEQ ID NO: 1193 | LC CDR3 (Chothia) | HQWSFYPT |
| SEQ ID NO: 1191 | LC CDR1 (Combined) | SANSTVGYIH |
| SEQ ID NO: 1192 | LC CDR2 (Combined) | TTSNLAS |
| SEQ ID NO: 1193 | LC CDR3(Combined) | HQWSFYPT |
| SEQ ID NO: 1195 | VL | QIVLTQSPAIMSASLGEEIALTCSANSTVGYIHW |
| | | YQQKSGTSPKLLIYTTSNLASGVPSRFSGSGSGTF |
| | | YSLTISSVEAEDAADYFCHQWSFYPTFGGGTKLE |
| | | IK |
| SEQ ID NO: 1196 | VH | QIQLQQSGPEVVKPGASVQISCKASGYTFSGSW |
| | | MNWVKQRPGKGLEWIGRIYPGDGDTDYSGKFK |
| | | GRATLTADKSSSTAYMRLSSLTSEDSAVYFCARS |
| | | GYFNYVPVFDYWGQGTTLSVSS |

VH for JOVI-3 (humanized) also referred to as Antibody K-H
Binds to human TCRVβ 28

| | | |
|---|---|---|
| SEQ ID NO: 1197 | VH - 1 | QIQLVQSGAEVKKPGASVKVSCKASGYTFSGSW |
| | | MNWVRQAPGQGLEWIGRIYPGDGDTDYSGKFK |
| | | GRATLTADKSTSTAYMELSSLRSEDTAVYYCAR |
| | | SGYFNYVPVFDYWGQGTTVTVSS |
| SEQ ID NO: 1198 | VH - 2 | QIQLVQSGAEVKKPGSSVKVSCKASGYTFSGSW |
| | | MNWVRQAPGQGLEWIGRIYPGDGDTDYSGKFK |
| | | GRATLTADKSTSTAYMELSSLRSEDTAVYYCAR |
| | | SGYFNYVPVFDYWGQGTTVTVSS |
| SEQ ID NO: 1199 | VH - 3 | EIQLVQSGAEVKKPGESLKISCKASGYTFSGSWM |
| | | NWVRQMPGKGLEWIGRIYPGDGDTDYSGKFKG |
| | | QATLSADKSISTAYLQWSSLKASDTAMYYCARS |
| | | GYFNYVPVFDYWGQGTTVTVSS |
| SEQ ID NO: 1200 | VH - 4 | QIQLVQSGSELKKPGASVKVSCKASGYTFSGSW |
| | | MNWVRQAPGQGLEWIGRIYPGDGDTDYSGKFK |
| | | GRAVLSADKSVSTAYLQISSLKAEDTAVYYCAR |
| | | SGYFNYVPVFDYWGQGTTVTVSS |
| SEQ ID NO: 1201 | VH - 5 | QIQLVQSGSELKKPGASVKVSCKASGYTFSGSW |
| | | MNWVRQAPGQGLEWIGRIYPGDGDTDYSGKFK |
| | | GRAVLSADKSVSMAYLQISSLKAEDTAVYYCAR |
| | | SGYFNYVPVFDYWGQGTTVTVSS |
| SEQ ID NO: 1202 | VH - 6 | EIQLVESGGGLVQPGRSLRLSCTASGYTFSGSW |
| | | MNWVRQAPGKGLEWIGRIYPGDGDTDYSGKFK |
| | | GRATLSADKSKSIAYLQMNSLKTEDTAVYYCAR |
| | | SGYFNYVPVFDYWGQGTTVTVSS |

VL for JOVI-3 (humanized) also referred to as Antibody K-H
Binds to human TCRVβ 28

| | | |
|---|---|---|
| SEQ ID NO: 1203 | VL - 1 | EIVLTQSPATLSLSPGERATLSCSANSTVGYIHW |
| | | YQQKPGQAPKLLIYTTSNLASGIPARFSGSGSGT |
| | | DYTLTISSLEPEDFAVYFCHQWSFYPTFGQGTKL |
| | | EIK |
| SEQ ID NO: 1204 | VL - 2 | DIQLTQSPSFLSASVGDRVTITCSANSTVGYIHW |
| | | YQQKPGKAPKLLIYTTSNLASGVPSRFSGSGSGT |
| | | EYTLTISSLQPEDFATYFCHQWSFYPTFGQGTKL |
| | | EIK |
| SEQ ID NO: 1205 | VL - 3 | EIVLTQSPATLSLSPGERATLSCSANSTVGYIHW |
| | | YQQKPGQAPKLLIYTTSNLASGIPARFSGSGPGT |
| | | DYTLTISSLEPEDFAVYFCHQWSFYPTFGQGTKL |
| | | EIK |
| SEQ ID NO: 1206 | VL - 4 | DIVLTQSPDSLAVSLGERATINCSANSTVGYIHW |
| | | YQQKPGQPPKLLIYTTSNLASGVPDRFSGSGSGT |
| | | DYTLTISSLQAEDVAVYFCHQWSFYPTFGQGTK |
| | | LEIK |
| SEQ ID NO: 1207 | VL - 5 | EIVLTQSPDFQSVTPKEKVTITCSANSTVGYIHW |
| | | YQQKPDQSPKLLIYTTSNLASGVPSRFSGSGSGT |
| | | DYTLTINSLEAEDAATYFCHQWSFYPTFGQGTK |
| | | LEIK |

ZOE (murine), also referred to as BJ1538 or as Antibody L
Binds to human TCRVβ 4-1,4-2,4-3

| | | |
|---|---|---|
| SEQ ID NO: 1208 | HC CDR1 (Kabat) | DYYMY |
| SEQ ID NO: 1209 | HC CDR2 (Kabat) | TISGGGSYTYSPDSVKG |
| SEQ ID NO: 1210 | HC CDR3 (Kabat) | ERDIYYGNFNAMVY |
| SEQ ID NO: 1211 | HC CDR1 (Chothia) | GFTFSDY |
| SEQ ID NO: 1212 | HC CDR2 (Chothia) | SGGGSY |
| SEQ ID NO: 1210 | HC CDR3 (Chothia) | ERDIYYGNFNAMVY |
| SEQ ID NO: 1213 | HC CDR1 (Combined) | GFTFSDYYMY |
| SEQ ID NO: 1209 | HC CDR2 (Combined) | TISGGGSYTYSPDSVKG |
| SEQ ID NO: 1210 | HC CDR3(Combined) | ERDIYYGNFNAMVY |
| SEQ ID NO: 1214 | LC CDR1 (Kabat) | RASKSVSTSGYSYMH |
| SEQ ID NO: 1215 | LC CDR2 (Kabat) | LASNLES |
| SEQ ID NO: 1216 | LC CDR3 (Kabat) | QHSRDLPWT |
| SEQ ID NO: 1217 | LC CDR1 (Chothia) | SKSVSTSGYSY |
| SEQ ID NO: 1215 | LC CDR2 (Chothia) | LASNLES |
| SEQ ID NO: 1216 | LC CDR3 (Chothia) | QHSRDLPWT |
| SEQ ID NO: 1214 | LC CDR1 (Combined) | RASKSVSTSGYSYMH |
| SEQ ID NO: 1215 | LC CDR2 (Combined) | LASNLES |
| SEQ ID NO: 1216 | LC CDR3(Combined) | QHSRDLPWT |
| SEQ ID NO: 1218 | VL | DIVLTQSPVSLTVSLGQRATISCRASKSVSTSGYS |
| | | YMHWYQQKPGQPPKLLIYLASNLESGVPARFSG |
| | | SGSGTDFTLNIHPVEEEDAATYYCQHSRDLPWTF |
| | | GGGTKLEIK |
| SEQ ID NO: 1219 | VH | EVQLVESGGGLVKPGGSLKLSCAASGFTFSDYY |
| | | MYWVRQTPEKRLEWVATISGGGSYTYSPDSVK |
| | | GRFTISRDNAKNNLYLQMSSLRSEDTAMYFCAR |
| | | ERDIYYGNFNAMVYWGRGTSVTVSS |

VH for ZOE (humanized) also referred to as Antibody L-H
Binds to human TCRVβ 4-1,4-2,4-3

| | | |
|---|---|---|
| SEQ ID NO: 1220 | VH - 1 | EVQLLESGGGLVQPGGSLRLSCAASGFTFSDYY |
| | | MYWVRQAPGKGLEWVATISGGGSYTYSPDSVK |
| | | GRFTISRDNSKNTLYLQMNSLRAEDTAVYYCAR |
| | | ERDIYYGNFNAMVYWGRGTLVTVSS |
| SEQ ID NO: 1221 | VH - 2 | EVQLVESGGGLVQPGGSLRLSCAASGFTFSDYY |
| | | MYWVRQAPGKGLEWVATISGGGSYTYSPDSVK |
| | | GRFTISRDNAKNSLYLQMNSLRAEDTAVYYCAR |
| | | ERDIYYGNFNAMVYWGRGTLVTVSS |
| SEQ ID NO: 1222 | VH - 3 | QVQLVESGGGVVQPGRSLRLSCAASGFTFSDY |
| | | YMYWVRQAPGKGLEWVATISGGGSYTYSPDS |
| | | VKGRFTISRDNSKNTLYLQMNSLRAEDTAVYY |
| | | CARERDIYYGNFNAMVYWGRGTLVTVSS |
| SEQ ID NO: 1223 | VH - 4 | QVQLVESGGGLVKPGGSLRLSCAASGFTFSDYY |
| | | MYWIRQAPGKGLEWVATISGGGSYTYSPDSVK |
| | | GRFTISRDNAKNSLYLQMNSLRAEDTAVYYCAR |
| | | ERDIYYGNFNAMVYWGRGTLVTVSS |

VL for ZOE (humanized) also referred to as Antibody L-H
Binds to human TCRVβ 4-1,4-2,4-3

| | | |
|---|---|---|
| SEQ ID NO: 1224 | VL - 1 | EIVLTQSPGTLSLSPGERATLSCRASKSVSTSGYS |
| | | YMHWYQQKPGQAPRLLIYLASNLESGIPDRFSG |
| | | SGSGTDFTLTISRLEPEDFAVYYCQHSRDLPWTF |
| | | GGGTKVEIK |
| SEQ ID NO: 1225 | VL - 2 | EIVLTQSPATLSLSPGERATLSCRASKSVSTSGYS |
| | | YMHWYQQKPGQAPRLLIYLASNLESGIPARFSG |
| | | SGSGTDFTLTISSLEPEDFAVYYCQHSRDLPWTF |
| | | GGGTKVEIK |
| SEQ ID NO: 1226 | VL - 3 | DIQLTQSPSTLSASVGDRVTITCRASKSVSTSGYS |
| | | YMHWYQQKPGKAPKLLIYLASNLESGVPSRFSG |
| | | SGSGTEFTLTISSLQPDDFATYYCQHSRDLPWTF |
| | | GGGTKVEIK |
| SEQ ID NO: 1227 | VL - 4 | AIQLTQSPSSLSASVGDRVTITCRASKSVSTSGYS |
| | | YMHWYQQKPGKAPKLLIYLASNLESGVPSRFSG |
| | | SGSGTDFTLTISSLQPEDFATYYCQHSRDLPWTF |
| | | GGGTKVEIK |

Anti-TCRvb19 (murine), also referred to as BJ1465; or Antibody M
Binds to human TCRVβ 19

| | | |
|---|---|---|
| SEQ ID NO: 1229 | HC CDR1 (Kabat) | GYFWN |
| SEQ ID NO: 1230 | HC CDR2 (Kabat) | YISYDGSNNYNPSLKN |
| SEQ ID NO: 1231 | HC CDR3 (Kabat) | PSPGTGYAVDY |
| SEQ ID NO: 1232 | HC CDR1 (Chothia) | GYSITSGY |
| SEQ ID NO: 1233 | HC CDR2 (Chothia) | SYDGSN |
| SEQ ID NO: 1231 | HC CDR3 (Chothia) | PSPGTGYAVDY |
| SEQ ID NO: 1234 | HC CDR1 (Combined) | GYSITSGYFWN |
| SEQ ID NO: 1230 | HC CDR2 (Combined) | YISYDGSNNYNPSLKN |
| SEQ ID NO: 1231 | HC CDR3(Combined) | PSPGTGYAVDY |
| SEQ ID NO: 1235 | LC CDR1 (Kabat) | RSSQSLVHSNGNTYLH |
| SEQ ID NO: 1236 | LC CDR2 (Kabat) | KVSNRFS |
| SEQ ID NO: 1237 | LC CDR3 (Kabat) | SQSTHVPFT |
| SEQ ID NO: 1238 | LC CDR1 (Chothia) | SQSLVHSNGNTY |
| SEQ ID NO: 1236 | LC CDR2 (Chothia) | KVSNRFS |
| SEQ ID NO: 1237 | LC CDR3 (Chothia) | SQSTHVPFT |
| SEQ ID NO: 1235 | LC CDR1 (Combined) | RSSQSLVHSNGNTYLH |
| SEQ ID NO: 1236 | LC CDR2 (Combined) | KVSNRFS |
| SEQ ID NO: 1237 | LC CDR3(Combined) | SQSTHVPFT |
| SEQ ID NO: 1239 | VL | NVVMTQTPLSLPVSLGDQASISCRSSQSLVHSNG |
| | | NTYLHWYLQKPGQSPKFLIYKVSNRFSGVPDRF |
| | | SGGGSGTEFTLKISRVEAEDLGVYFCSQSTHVPF |
| | | TFGSGTKLEIK |
| SEQ ID NO: 1240 | \|VH | NVQLQESGPGLVKPSQSLSLTCSVAGYSITSGYF |
| | | WNWIRQFPGNKLEWMGYISYDGSNNYNPSLKN |
| | | RISITRDTSKNQFFLKLNSVTTEDTATYYCASPSP |
| | | GTGYAVDYWGQGTSVTVSS |

VH for Anti-TCRvb19 (humanized) also referred to as Antibody M-H
Binds to human TCRVβ 19

| | | |
|---|---|---|
| SEQ ID NO: 1241 | VH - 1 | QVQLQESGPGLVKPSETLSLTCTVSGYSITSGYF |
| | | WNWIRQPPGKGLEWIGYISYDGSNNYNPSLKNR |
| | | VTISRDTSKNQFSLKLSSVTAADTAVYYCASPSP |
| | | GTGYAVDYWGQGTLVTVSS |
| SEQ ID NO: 1242 | VH - 2 | QVQLQESGPGLVKPSETLSLTCTVSGYSITSGYF |
| | | WNWIRQPPGKGLEWIGYISYDGSNNYNPSLKNR |
| | | VTISRDTSKNQFSLKLSSVTAADTAVYYCASPSP |
| | | GTGYAVDYWGQGTLVTVSS |
| SEQ ID NO: 1243 | VH - 3 | QVQLVESGGGLVQPGGSLRLSCSVSGYSITSGYF |
| | | WNWVRQAPGKGLEWVGYISYDGSNNYNPSLK |
| | | NRFTISRDTSKNTFYLQMNSLRAEDTAVYYCAS |
| | | PSPGTGYAVDYWGQGTLVTVSS |

VL for Anti-TCRvb19 (humanized) also referred to as Antibody M-H
Binds to human TCRVβ 19

| | | |
|---|---|---|
| SEQ ID NO: 1244 | VL - 1 | VVMTQSPGTLSLSPGERATLSCRSSQSLVHSNGN |
| | | TYLHWYQQKPGQAPRFLIYKVSNRFSGIPDRFSG |
| | | SGSGTDFTLTISRLEPEDFAVYFCSQSTHVPFTFG |
| | | QGTKLEIK |
| SEQ ID NO: 1245 | VL - 2 | EVVMTQSPATLSLSPGERATLSCRSSQSLVHSNG |
| | | NTYLHWYQQKPGQAPRFLIYKVSNRFSGIPARFS |
| | | GSGSGTDFTLTISSLEPEDFAVYFCSQSTHVPFTF |
| | | GQGTKLEIK |
| SEQ ID NO: 1246 | VL - 3 | EVVMTQSPATLSVSPGERATLSCRSSQSLVHSNG |
| | | NTYLHWYQQKPGQAPRFLIYKVSNRFSGIPARFS |
| | | GSGSGTEFTLTISSLQSEDFAVYFCSQSTHVPFTF |
| | | GQGTKLEIK |
| SEQ ID NO: 1247 | VL - 4 | DVQMTQSPSSLSASVGDRVTITCRSSQSLVHSNG |
| | | NTYLHWYQQKPGKAPKFLIYKVSNRFSGVPSRF |
| | | SGSGSGTDFTFTISSLQPEDIATYFCSQSTHVPFTF |
| | | GQGTKLEIK |

BL37.2 (murine), also referred to as BJ1539 or Antibody N
Binds to human TCRVβ 9

| | | |
|---|---|---|
| SEQ ID NO: 1248 | HC CDR1 (Kabat) | DYIVH |
| SEQ ID NO: 1249 | HC CDR2 (Kabat) | WINTYTGTPTYADDFEG |
| SEQ ID NO: 1250 | HC CDR3 (Kabat) | SWRRGIRGIGFDY |
| SEQ ID NO: 1251 | HC CDR1 (Chothia) | GYTFTDY |
| SEQ ID NO: 1252 | HC CDR2 (Chothia) | NTYTGT |
| SEQ ID NO: 1250 | HC CDR3 (Chothia) | SWRRGIRGIGFDY |
| SEQ ID NO: 1253 | HC CDR1 (Combined) | GYTFTDYIVH |
| SEQ ID NO: 1249 | HC CDR2 (Combined) | WINTYTGTPTYADDFEG |
| SEQ ID NO: 1250 | HC CDR3(Combined) | SWRRGIRGIGFDY |
| SEQ ID NO: 1254 | LC CDR1 (Kabat) | KASKSINKYLA |
| SEQ ID NO: 1255 | LC CDR2 (Kabat) | DGSTLQS |
| SEQ ID NO: 1256 | LC CDR3 (Kabat) | QQHNEYPPT |
| SEQ ID NO: 1257 | LC CDR1 (Chothia) | SKSINKY |
| SEQ ID NO: 1255 | LC CDR2 (Chothia) | DGSTLQS |
| SEQ ID NO: 1256 | LC CDR3 (Chothia) | QQHNEYPPT |
| SEQ ID NO: 1254 | LC CDR1 (Combined) | KASKSINKYLA |
| SEQ ID NO: 1255 | LC CDR2 (Combined) | DGSTLQS |
| SEQ ID NO: 1256 | LC CDR3(Combined) | QQHNEYPPT |
| SEQ ID NO: 1258 | VL | DVQMTQSPYNLAASPGESVSINCKASKSINKYLA |
| | | WYQQKPGKPNKLLIYDGSTLQSGIPSRFSGSGSG |
| | | TDFTLTIRGLEPEDFGLYYCQQHNEYPPTFGAGT |
| | | KLELK |
| SEQ ID NO: 1259 | VH | QLQLVQSGPELREPGESVKISCKASGYTFTDYIV |
| | | HWVKQAPGKGLKWMGWINTYTGTPTYADDFE |
| | | GRFVFSLEASASTANLQISNLKNEDTATYFCARS |
| | | WRRGIRGIGFDYWGQGVMVTVSS |

VH for BL37.2 (humanized) also referred to as Antibody N-H
Binds to human TCRVβ 9

| | | |
|---|---|---|
| SEQ ID NO: 1260 | VH - 1 | QLQLVQSGAEVKKPGASVKVSCKASGYTFTDYI |
| | | VHWVRQAPGQGLEWMGWINTYTGTPTYADDF |
| | | EGWVTMTLDASISTAYMELSRLRSDDTAVYYC |
| | | ARSWRRGIRGIGFDYWGQGTMVTVSS |
| SEQ ID NO: 1261 | VH - 2 | QLQLVQSGAEVKKPGASVKVSCKASGYTFTDYI |
| | | VHWVRQAPGQGLEWMGWINTYTGTPTYADDF |
| | | EGRVTMTLDASTSTAYMELSSLRSEDTAVYYCA |
| | | RSWRRGIRGIGFDYWGQGTMVTVSS |
| SEQ ID NO: 1262 | VH - 3 | QLQLVQSGAEVKKPGASVKVSCKASGYTFTDYI |
| | | VHWVRQAPGQRLEWMGWINTYTGTPTYADDF |
| | | EGRVTITLDASASTAYMELSSLRSEDMAVYYCA |
| | | RSWRRGIRGIGFDYWGQGTMVTVSS |
| SEQ ID NO: 1263 | VH - 4 | QLQLVQSGAEVKKPGASVKVSCKASGYTFTDYI |
| | | VHWVRQATGQGLEWMGWINTYTGTPTYADDF |
| | | EGRVTMTLNASISTAYMELSSLRSEDTAVYYCA |
| | | RSWRRGIRGIGFDYWGQGTMVTVSS |

VL for BL37.2 (humanized) also referred to as Antibody N-H
Binds to human TCRVβ 9

| | | |
|---|---|---|
| SEQ ID NO: 1264 | VL - 1 | EVVMTQSPGTLSLSPGERATLSCKASKSINKYLA |
| | | WYQQKPGQAPRLLIYDGSTLQSGIPDRFSGSGSG |
| | | TDFTLTISRLEPEDFAVYYCQQHNEYPPTFGQGT |
| | | KLEIK |
| SEQ ID NO: 1265 | VL - 2 | EVVMTQSPATLSLSPGERATLSCKASKSINKYLA |
| | | WYQQKPGQAPRLLIYDGSTLQSGIPARFSGSGSG |
| | | TDFTLTISSLEPEDFAVYYCQQHNEYPPTFGQGT |
| | | KLEIK |
| SEQ ID NO: 1266 | VL - 3 | DVQMTQSPSSLSASVGDRVTITCKASKSINKYLA |
| | | WYQQKPGKAPKLLIYDGSTLQSGVPSRFSGSGS |
| | | GTDFTLTISSLQPEDFATYYCQQHNEYPPTFGQG |
| | | TKLEIK |
| SEQ ID NO: 1267 | VL - 4 | AVRMTQSPSSFSASTGDRVTITCKASKSINKYLA |
| | | WYQQKPGKAPKLLIYDGSTLQSGVPSRFSGSGS |
| | | GTDFTLTISCLOSEDFATYYCQQHNEYPPTFGQG |
| | | TKLEIK |

IG125 (murine) binds to TRVβ 11-2; also referred to as Antibody O

| | | |
|---|---|---|
| SEQ ID NO: 1268 | HC CDR1 (Kabat) | NYGVH |
| SEQ ID NO: 1269 | HC CDR2 (Kabat) | VIWSDGSTDYDTAFIS |
| SEQ ID NO: 1270 | HC CDR3 (Kabat) | RAVVADFDY |
| SEQ ID NO: 1271 | HC CDR1 (Chothia) | GFSLTN |
| SEQ ID NO: 1272 | HC CDR2 (Chothia) | VIWSDGSTD |
| SEQ ID NO: 1270 | HC CDR3 (Chothia) | RAVVADFDY |
| SEQ ID NO: 1273 | HC CDR1 (combined) | GFSLTNYGVH |
| SEQ ID NO: 1269 | HC CDR2 (combined) | VIWSDGSTDYDTAFIS |
| SEQ ID NO: 1270 | HC CDR3 (combined) | RAVVADFDY |
| SEQ ID NO: 1274 | VH | QVQLKQSGPGLLQPSQSLSITCTVSGFSLTNYGV |
| | | HWVRQSPGKGLEWLGVIWSDGSTDYDTAFISRL |
| | | SISKDNSKSQVFFKLNSLQADDTAIYYCARRAV |
| | | VADFDYWGQGTTLTVSS |
| SEQ ID NO:1275 | LC CDR1 (Kabat) | KASKEVTIFGSISALH |
| SEQ ID NO: 1276 | LC CDR2 (Kabat) | NGAKLES |
| SEQ ID NO: 1277 | LC CDR3 (Kabat) | LQNKEVPFT |
| SEQ ID NO:1275 | LC CDR1 (Chothia) | KASKEVTIFGSISALH |
| SEQ ID NO:1276 | LC CDR2 (Chothia) | NGAKLES |
| SEQ ID NO: 1277 | LC CDR3 (Chothia) | LQNKEVPFT |
| SEQ ID NO: 1275 | LC CDR1 (combined) | KASKEVTIFGSISALH |
| SEQ ID NO: 1276 | LC CDR2 (combined) | NGAKLES |
| SEQ ID NO: 1277 | LC CDR3 (combined) | LQNKEVPFT |
| SEQ ID NO: 1278 | VL | DIVLTQSPASLAVSLGQKATISCKASKEVTIFGSI |
| | | SALHWYQQKPGQPPKLIYNGAKLESGVSARFS |
| | | DSGSQNRSPFGNQLSFTLTIAPVEADDAATYYC |
| | | LQNKEVPFTFGSGTKLEIK |

VL for IG125 (humanized) also referred to as Antibody O-H
binds to TRβV 11-2

| | | |
|---|---|---|
| SEQ ID NO: 1279 | VL-1 | DIVLTQSPDSLAVSLGERATINCKASKEVTIFGSI |
| | | SALHWYQQKPGQPPKLLYNGAKLESGVSARFG |
| | | VPDRFSRSGSGLDFTLTISSLQAEDVAVYYCLQ |
| | | NKEVPFTFGQGTKLEIK |
| SEQ ID NO: 1280 | VL-2 | EIVLTQSPDFQSVTPKEKVTITCKASKEVTIFGSI |
| | | SALHWYQQKPDQSPKLLYNGAKLESGVSARFG |
| | | VPSRFSRSGSGLDFTLTINSLEAEDAATYYCLQN |
| | | KEVPFTFGQGTKLEIK |
| SEQ ID NO: 1281 | VL-3 | AIQLTQSPSSLSASVGDRVTITCKASKEVTIFGSI |
| | | SALHWYQQKPGKAPKLLYNGAKLESGVSARF |
| | | GVPSRFSRSGSGLDFTLTISSLQPEDFATYYCLQ |
| | | NKEVPFTFGQGTKLEIK |
| SEQ ID NO: 1282 | VL-4 | DIVLTQTPLSLSVTPGQPASISCKASKEVTIFGSIS |
| | | ALHWYLQKPGQPPKLLYNGAKLESGVSARFGV |
| | | PDRFSRSGSGLDFTLKISRVEAEDVGVYYCLQN |
| | | KEVPFTFGQGTKLEIK |

VH for IG125 (humanized) also referred to as Antibody O-H
binds to TRVβ 11-2

| | | |
|---|---|---|
| SEQ ID NO: 1283 | VH-1 | QVTLKESGPVLVKPTETLTLTCTVSGFSLTNYG |
| | | VHWVRQPPGKALEWLGVIWSDGSTDYDTAFIS |
| | | RLTISKDNSKSQVVLTMTNMDPVDTATYYCAR |
| | | RAVVADFDYWGQGTTVTVSS |
| SEQ ID NO: 1284 | VH-2 | QVQLQESGPGLVKPSGTLSLTCAVSGFSLTNYG |
| | | VHWVRQPPGKGLEWLGVIWSDGSTDYDTAFIS |
| | | RLTISKDNSKSQVSLKLSSVTAADTAVYYCARR |
| | | AVVADFDYWGQGTTVTVSS |
| SEQ ID NO: 1285 | VH-3 | QVQLQQSGPGLVKPSQTLSLTCAVSGFSLTNYG |
| | | VHWVRQSPSRGLEWLGVIWSDGSTDYDTAFIS |
| | | RLTINKDNSKSQVSLQLNSVTPEDTAVYYCARR |
| | | AVVADFDYWGQGTTVTVSS |
| SEQ ID NO: 1286 | VH-4 | EVQLVESGGGLVQPGPSLRLSCTVSGFSLTNYG |
| | | VHWVRQAPGKGLEWLGVIWSDGSTDYDTAFIS |
| | | RLTISKDNSKSIVYLQMNSLKTEDTAVYYCARR |
| | | AVVADFDYWGQGTTVTVSS |
| SEQ ID NO: 1287 | VH-5 | EVQLVQSGAEVKKPGESLRISCKVSGFSLTNYG |
| | | VHWVRQMPGKGLEWLGVIWSDGSTDYDTAFI |
| | | SQLTISKDNSISTVYLQWSSLKASDTAMYYCAR |
| | | RAVVADFDYWGQGTTVTVSS |

MR5-2 (murine), Binds to human TCRVβ 13-2

| | | |
|---|---|---|
| SEQ ID NO: 1376 | SCFV (VH + VL) | QVQLQQSGTELMKPGASVKISCKASGYTFSNY |
| | | WIEWIKQRPGHGLEWVGEILPGAGPTNYNEKF |
| | | KGKATFTADSSSNTAYMQLSSLTSEDSAVYYC |
| | | ARTDYDYDWFAYWGQGTLVTVSAGGGGSGG |
| | | GGSGGGGSGGGGSDIVMSQSPSSLAVSVGEKV |
| | | TMSCKSSQSLLYSGNQKNYLAWYQQKPGQSPK |
| | | LLIYWASTRESGVPDRFTGSGSGTDFTLTINSVK |
| | | AEDLTVYYCQQYYGYPRTFGGGTKVEIK |

### Anti-TCRVβ Antibody Effector Function and Fc Variants

In some embodiments, an anti-TCRVβ antibody disclosed herein comprises an Fc region, e.g., as described herein. In some embodiments, the Fc region is a wildtype Fc region, e.g., a wildtype human Fc region. In some embodiments, the Fc region comprises a variant, e.g., an Fc region comprising an addition, substitution, or deletion of at least one amino acid residue in the Fc region which results in, e.g., reduced or ablated affinity for at least one Fc receptor.

The Fc region of an antibody interacts with a number of receptors or ligands including Fc Receptors (e.g., FcγRI, FcγRIIA, FcγRIIIA), the complement protein CIq, and other molecules such as proteins A and G. These interactions are essential for a variety of effector functions and downstream signaling events including: antibody dependent cell-mediated cytotoxicity (ADCC), Antibody-dependent cellular phagocytosis (ADCP) and complement dependent cytotoxicity (CDC).

In some embodiments, an anti-TCRVβ antibody comprising a variant Fc region has reduced, e.g., ablated, affinity for an Fc receptor, e.g., an Fc receptor described herein. In some embodiments, the reduced affinity is compared to an otherwise similar antibody with a wildtype Fc region.

In some embodiments, an anti-TCRVβ antibody comprising a variant Fc region has one or more of the following properties: (1) reduced effector function (e.g., reduced ADCC, ADCP and/or CDC); (2) reduced binding to one or more Fc receptors; and/or (3) reduced binding to C1q complement. In some embodiments, the reduction in any one, or all of properties (1)-(3) is compared to an otherwise similar antibody with a wildtype Fc region.

In some embodiments, an anti-TCRVβ antibody comprising a variant Fc region has reduced affinity to a human Fc receptor, e.g., FcγR I, FcγR II and/or FcγR III. In some embodiments, the anti-TCRVβ antibody comprising a variant Fc region comprises a human IgG1 region or a human IgG4 region.

In some embodiments, an anti-TCRVβ antibody comprising a variant Fc region activates and/or expands T cells, e.g., as described herein. In some embodiments, an anti-TCRVβ antibody comprising a variant Fc region has a cytokine profile described herein, e.g., a cytokine profile that differs from a cytokine profile of a T cell engager that binds to a receptor or molecule other than a TCRβV region (“a non-TCRβV-binding T cell engager”). In some embodiments, the non-TCRβV-binding T cell engager comprises an antibody that binds to a CD3 molecule (e.g., CD3 epsilon (CD3e) molecule); or a TCR alpha (TCRα) molecule.

Exemplary Fc region variants are provided in Table 14 and also disclosed in Saunders 0, (2019) Frontiers in Immunology; vol 10, article1296, the entire contents of which is hereby incorporated by reference.

In some embodiments, an anti-TCRVβ antibody disclosed herein comprises any one or all, or any combination of Fc region variants, e.g., mutations, disclosed in Table 14. In some embodiments, an anti-TCRVβ antibody disclosed herein comprise an Asn297Ala (N297A) mutation. In some embodiments, an anti-TCRVβ antibody disclosed herein comprise a Leu234Ala/Leu235Ala (LALA) mutation.

TABLE 14
Exemplary Fc modifications

| Modification or mutation | Altered effector function |
|---|---|
| Leu235Glu | ADCC; |
| Leu234Ala/Leu235Ala (LALA) | ADCC; ADCP; CDC |
| Ser228Pro/Leu235Glu | |
| Leu234Ala/Leu235Ala/Pro329Gly | ADCP |
| Pro331Ser/Leu234Glu/Leu235Phe | CDC |
| Asp265Ala | ADCC, ADCP |
| Gly237Ala | ADCP |
| Glu318Ala | ADCP |
| Glu233Pro | |
| Gly236Arg/Leu328Arg | ADCC |
| His268Gln/Val309Leu/Ala330Ser/Pro331Ser | ADCC; ADCP; CDC |
| Val234Ala/Gly237Ala/Pro238Ser/ | ADCC; ADCP; CDC |
| His268Ala/Val309Leu/Ala330Ser/Pro331Ser | |
| Leu234Ala/L235Ala/Gly237Ala/P238Ser/ | ADCC; CDC |
| His268Ala/Ala330Ser/Pro331Ser | |
| Ala330Leu | CDC |
| Asp270Ala | CDC |
| Lys322Ala | CDC |
| Pro329Ala | CDC |
| Pro331Ala | CDC |
| Val264Ala | CDC |
| High mannose glycosylation | CDC |
| Phe241Ala | CDC |
| Asn297Ala or Gly or Gln | ADCC; ADCP; CDC |
| S228P/Phe234Ala/Leu235Ala | ADCC; CDC |

### Natural Killer Cell Engagers

Natural Killer (NK) cells recognize and destroy tumors and virus-infected cells in an antibody-independent manner. The regulation of NK cells is mediated by activating and inhibiting receptors on the NK cell surface. One family of activating receptors is the natural cytotoxicity receptors (NCRs) which include NKp30, NKp44 and NKp46. The NCRs initiate tumor targeting by recognition of heparan sulfate on cancer cells. NKG2D is a receptor that provides both stimulatory and costimulatory innate immune responses on activated killer (NK) cells, leading to cytotoxic activity. DNAM1 is a receptor involved in intercellular adhesion, lymphocyte signaling, cytotoxicity and lymphokine secretion mediated by cytotoxic T-lymphocyte (CTL) and NK cell. DAP10 (also known as HCST) is a transmembrane adapter protein which associates with KLRK1 to form an activation receptor KLRK1-HCST in lymphoid and myeloid cells; this receptor plays a major role in triggering cytotoxicity against target cells expressing cell surface ligands such as MHC class I chain-related MICA and MICB, and U(optionally L1)6-binding proteins (ULBPs); it KLRK1-HCST receptor plays a role in immune surveillance against tumors and is required for cytolysis of tumors cells; indeed, melanoma cells that do not express KLRK1 ligands escape from immune surveillance mediated by NK cells. CD16 is a receptor for the Fc region of IgG, which binds complexed or aggregated IgG and also monomeric IgG and thereby mediates antibody-dependent cellular cytotoxicity (ADCC) and other antibody-dependent responses, such as phagocytosis.

The present disclosure provides, inter alia, multispecific (e.g., bi-, tri-, quad-specific) or multifunctional molecules, that are engineered to contain one or more NK cell engagers that mediate binding to and/or activation of an NK cell. Accordingly, in some embodiments, the NK cell engager is selected from an antigen binding domain or ligand that binds to (e.g., activates): NKp30, NKp40, NKp44, NKp46, NKG2D, DNAM1, DAP10, CD16 (e.g., CD16a, CD16b, or both), CRTAM, CD27, PSGL1, CD96, CD100 (SEMA4D), NKp80, CD244 (also known as SLAMF4 or 2B4), SLAMF6, SLAMF7, KIR2DS2, KIR2DS4, KIR3DS1, KIR2DS3, KIR2DS5, KIR2DS1, CD94, NKG2C, NKG2E, or CD160.

In some embodiments, the NK cell engager is an antigen binding domain that binds to NKp30 (e.g., NKp30 present, e.g., expressed or displayed, on the surface of an NK cell) and comprises any CDR amino acid sequence, framework region (FWR) amino acid sequence, or variable region amino acid sequence disclosed in Tables 7, 8, 35, 36, 9, 10, or 34. In some embodiments, the NK cell engager is an antigen binding domain that binds to NKp30 (e.g., NKp30 present, e.g., expressed or displayed, on the surface of an NK cell) and comprises any CDR amino acid sequence, framework region (FWR) amino acid sequence, or variable region amino acid sequence disclosed in U.S. Pat. Nos. 6,979,546, 9,447,185, PCT Application No. WO2015121383A1, PCT Application No.

WO2016110468A1, PCT Application No. WO2004056392A1, or U.S. Application Publication No. US20070231322A1, the sequences of which are hereby incorporated by reference. In some embodiments, binding of the NK cell engager, e.g., antigen binding domain that binds to NKp30, to the NK cell activates the NK cell. An antigen binding domain that binds to NKp30 (e.g., NKp30 present, e.g., expressed or displayed, on the surface of an NK cell) may be said to target NKp30, the NK cell, or both.

In some embodiments, the antigen binding domain that binds to NKp30 comprises one or more CDRs (e.g., VHCDR1, VHCDR2, VHCDR3, VLCDR1, VLCDR2, and/or VLCDR3) disclosed in Table 7, Table 34, or Table 8, or a sequence having at least 85%, 90%, 95%, or 99% identity thereto. In some embodiments, the antigen binding domain that binds to NKp30 comprises one or more framework regions (e.g., VHFWR1, VHFWR2, VHFWR3, VHFWR4, VLFWR1, VLFWR2, VLFWR3, and/or VLFWR4) disclosed in Table 7, Table 34, or Table 8, or a sequence having at least 85%, 90%, 95%, or 99% identity thereto.

In some embodiments, the antigen binding domain that binds to NKP30 comprises one or more CDRs (e.g., VHCDR1, VHCDR2, VHCDR3, VLCDR1, VLCDR2, and/or VLCDR3) disclosed in Table 35 and/or Table 36, or a sequence having at least 85%, 90%, 95%, or 99% identity thereto. In some embodiments, the antigen binding domain that binds to NKP30 comprises one or more framework regions (e.g., VHFWR1, VHFWR2, VHFWR3, VHFWR4, VLFWR1, VLFWR2, VLFWR3, and/or VLFWR4) disclosed in Table 35 and/or Table 36, or a sequence having at least 85%, 90%, 95%, or 99% identity thereto.

In some embodiments, the antigen binding domain that binds to NKp30 comprises a VH and/or a VL disclosed in Table 9, or a sequence having at least 85%, 90%, 95%, or 99% identity thereto. In some embodiments, any of the VH domains disclosed in Table 9 may be paired with any of the VL domains disclosed in Table 9 to form the antigen binding domain that binds to NKp30. In some embodiments, the antigen binding domain that binds to NKp30 comprises an amino acid sequence disclosed in Table 10, or a sequence having at least 85%, 90%, 95%, or 99% identity thereto.

In some embodiments, the antigen binding domain that binds to NKp30 comprises a VH comprising a heavy chain complementarity determining region 1 (VHCDR1), a VHCDR2, and a VHCDR3, and a VL comprising a light chain complementarity determining region 1 (VLCDR1), a VLCDR2, and a VLCDR3.

In some embodiments, the VHCDR1, VHCDR2, and VHCDR3 comprise the amino acid sequences of SEQ ID NOs: 7313, 6001, and 7315, respectively (or a sequence having at least 85%, 90%, 95%, or 99% identity thereto). In some embodiments, the VHCDR1, VHCDR2, and VHCDR3 comprise the amino acid sequences of SEQ ID NOs: 7313, 6001, and 6002, respectively (or a sequence having at least 85%, 90%, 95%, or 99% identity thereto). In some embodiments, the VHCDR1, VHCDR2, and VHCDR3 comprise the amino acid sequences of SEQ ID NOs: 7313, 6008, and 6009, respectively (or a sequence having at least 85%, 90%, 95%, or 99% identity thereto). In some embodiments, the VHCDR1, VHCDR2, and VHCDR3 comprise the amino acid sequences of SEQ ID NOs: 7313, 7385, and 7315, respectively (or a sequence having at least 85%, 90%, 95%, or 99% identity thereto). In some embodiments, the VHCDR1, VHCDR2, and VHCDR3 comprise the amino acid sequences of SEQ ID NOs: 7313, 7318, and 6009, respectively (or a sequence having at least 85%, 90%, 95%, or 99% identity thereto).

In some embodiments, the VLCDR1, VLCDR2, and VLCDR3 comprise the amino acid sequences of SEQ ID NOs: 7326, 7327, and 7329, respectively (or a sequence having at least 85%, 90%, 95%, or 99% identity thereto). In some embodiments, the VLCDR1, VLCDR2, and VLCDR3 comprise the amino acid sequences of SEQ ID NOs: 6063, 6064, and 7293, respectively (or a sequence having at least 85%, 90%, 95%, or 99% identity thereto). In some embodiments, the VLCDR1, VLCDR2, and VLCDR3 comprise the amino acid sequences of SEQ ID NOs: 6070, 6071, and 6072, respectively (or a sequence having at least 85%, 90%, 95%, or 99% identity thereto). In some embodiments, the VLCDR1, VLCDR2, and VLCDR3 comprise the amino acid sequences of SEQ ID NOs: 6070, 6064, and 7321, respectively (or a sequence having at least 85%, 90%, 95%, or 99% identity thereto).

In some embodiments, the VHCDR1, VHCDR2, VHCDR3, VLCDR1, VLCDR2, and VLCDR3 comprise the amino acid sequences of SEQ ID NOs: 7313, 6001, 7315, 7326, 7327, and 7329, respectively (or a sequence having at least 85%, 90%, 95%, or 99% identity thereto). In some embodiments, the VHCDR1, VHCDR2, VHCDR3, VLCDR1, VLCDR2, and VLCDR3 comprise the amino acid sequences of SEQ ID NOs: 7313, 6001, 6002, 6063, 6064, and 7293, respectively (or a sequence having at least 85%, 90%, 95%, or 99% identity thereto). In some embodiments, the VHCDR1, VHCDR2, VHCDR3, VLCDR1, VLCDR2, and VLCDR3 comprise the amino acid sequences of SEQ ID NOs: 7313, 6008, 6009, 6070, 6071, and 6072, respectively (or a sequence having at least 85%, 90%, 95%, or 99% identity thereto). In some embodiments, the VHCDR1, VHCDR2, VHCDR3, VLCDR1, VLCDR2, and VLCDR3 comprise the amino acid sequences of SEQ ID NOs: 7313, 7385, 7315, 6070, 6064, and 7321, respectively (or a sequence having at least 85%, 90%, 95%, or 99% identity thereto). In some embodiments, the VHCDR1, VHCDR2, VHCDR3, VLCDR1, VLCDR2, and VLCDR3 comprise the amino acid sequences of SEQ ID NOs: 7313, 7318, 6009, 6070, 6064, and 7321, respectively (or a sequence having at least 85%, 90%, 95%, or 99% identity thereto).

In some embodiments, the VH comprises an amino acid sequence selected from the group consisting of SEQ ID NOs: 7298 or 7300-7304 (or a sequence having at least 85%, 90%, 95%, or 99% identity thereto) and/or the VL comprises an amino acid sequence selected from the group consisting of SEQ ID NOs: 7299 or 7305-7309 (or a sequence having at least 85%, 90%, 95%, or 99% identity thereto). In some embodiments, the VH and VL comprise the amino acid sequences of SEQ ID NOs: 7302 and 7305, respectively (or a sequence having at least 85%, 90%, 95%, or 99% identity thereto). In some embodiments, the VH and VL comprise the amino acid sequences of SEQ ID NOs: 7302 and 7309, respectively (or a sequence having at least 85%, 90%, 95%, or 99% identity thereto).

In some embodiments, the VH comprises an amino acid sequence selected from the group consisting of SEQ ID NOs: 6121 or 6123-6128 (or a sequence having at least 85%, 90%, 95%, or 99% identity thereto) and/or the VL comprises an amino acid sequence selected from the group consisting of SEQ ID NOs: 7294 or 6137-6141 (or a sequence having at least 85%, 90%, 95%, or 99% identity thereto). In some embodiments, the VH comprises an amino acid sequence selected from the group consisting of SEQ ID NOs: 6122 or 6129-6134 (or a sequence having at least 85%, 90%, 95%, or 99% identity thereto) and/or the VL comprises an amino acid sequence selected from the group consisting of SEQ ID NOs: 6136 or 6142-6147 (or a sequence having at least 85%, 90%, 95%, or 99% identity thereto). In some embodiments, the VH and VL comprise the amino acid sequences of SEQ ID NOs: 7295 and 7296, respectively (or a sequence having at least 85%, 90%, 95%, or 99% identity thereto). In some embodiments, the VH and VL comprise the amino acid sequences of SEQ ID NOs: 7297 and 7296, respectively (or a sequence having at least 85%, 90%, 95%, or 99% identity thereto). In some embodiments, the VH and VL comprise the amino acid sequences of SEQ ID NOs: 6122 and 6136, respectively (or a sequence having at least 85%, 90%, 95%, or 99% identity thereto).

In some embodiments, the antigen binding domain that binds to NKp30 comprises the amino acid sequence of SEQ ID NO: 7310 (or a sequence having at least 85%, 90%, 95%, or 99% identity thereto). In some embodiments, the antigen binding domain that binds to NKp30 comprises the amino acid sequence of SEQ ID NO: 7311 (or a sequence having at least 85%, 90%, 95%, or 99% identity thereto). In some embodiments, the antigen binding domain that binds to NKp30 comprises the amino acid sequence of SEQ ID NO: 6187, 6188, 6189 or 6190 (or a sequence having at least 85%, 90%, 95%, or 99% identity thereto).

In some embodiments, the antigen binding domain that targets NKp30 comprises a VH comprising a heavy chain complementarity determining region 1 (VHCDR1) amino acid sequence of SEQ ID NO: 6000 (or a sequence with no more than 1, 2, 3, or 4 mutations, e.g., substitutions, additions, or deletions), a VHCDR2 amino acid sequence of SEQ ID NO: 6001 (or a sequence with no more than 1, 2, 3, or 4 mutations, e.g., substitutions, additions, or deletions), and/or a VHCDR3 amino acid sequence of SEQ ID NO: 6002 (or a sequence with no more than 1, 2, 3, or 4 mutations, e.g., substitutions, additions, or deletions). In some embodiments, the NKp30 antigen binding domain comprises a VH comprising a VHCDR1 amino acid sequence of SEQ ID NO: 6000, a VHCDR2 amino acid sequence of SEQ ID NO: 6001, and/or a VHCDR3 amino acid sequence of SEQ ID NO: 6002.

In some embodiments, the antigen binding domain that targets NKp30 comprises a VL comprising a light chain complementarity determining region 1 (VLCDR1) amino acid sequence of SEQ ID NO: 6063 (or a sequence with no more than 1, 2, 3, or 4 mutations, e.g., substitutions, additions, or deletions), a VLCDR2 amino acid sequence of SEQ ID NO: 6064 (or a sequence with no more than 1, 2, 3, or 4 mutations, e.g., substitutions, additions, or deletions), and/or a VLCDR3 amino acid sequence of SEQ ID NO: 7293 (or a sequence with no more than 1, 2, 3, or 4 mutations, e.g., substitutions, additions, or deletions). In some embodiments, the antigen binding domain that targets NKp30 comprises a VL comprising a VLCDR1 amino acid sequence of SEQ ID NO: 6063, a VLCDR2 amino acid sequence of SEQ ID NO: 6064, and a VLCDR3 amino acid sequence of SEQ ID NO: 7293.

In some embodiments, the antigen binding domain that targets NKp30 comprises a VH comprising a heavy chain complementarity determining region 1 (VHCDR1) amino acid sequence of SEQ ID NO: 6000 (or a sequence with no more than 1, 2, 3, or 4 mutations, e.g., substitutions, additions, or deletions), a VHCDR2 amino acid sequence of SEQ ID NO: 6001 (or a sequence with no more than 1, 2, 3, or 4 mutations, e.g., substitutions, additions, or deletions), and/or a VHCDR3 amino acid sequence of SEQ ID NO: 6002 (or a sequence with no more than 1, 2, 3, or 4 mutations, e.g., substitutions, additions, or deletions), and a VL comprising a light chain complementarity determining region 1 (VLCDR1) amino acid sequence of SEQ ID NO: 6063 (or a sequence with no more than 1, 2, 3, or 4 mutations, e.g., substitutions, additions, or deletions), a VLCDR2 amino acid sequence of SEQ ID NO: 6064 (or a sequence with no more than 1, 2, 3, or 4 mutations, e.g., substitutions, additions, or deletions), and/or a VLCDR3 amino acid sequence of SEQ ID NO: 7293 (or a sequence with no more than 1, 2, 3, or 4 mutations, e.g., substitutions, additions, or deletions). In some embodiments, the NKp30 antigen binding domain comprises a VH comprising a VHCDR1 amino acid sequence of SEQ ID NO: 6000, a VHCDR2 amino acid sequence of SEQ ID NO: 6001, and/or a VHCDR3 amino acid sequence of SEQ ID NO: 6002, and a VL comprising a VLCDR1 amino acid sequence of SEQ ID NO: 6063, a VLCDR2 amino acid sequence of SEQ ID NO: 6064, and a VLCDR3 amino acid sequence of SEQ ID NO: 7293.

In some embodiments, the antigen binding domain that targets NKp30 comprises a VH comprising a heavy chain complementarity determining region 1 (VHCDR1) amino acid sequence of SEQ ID NO: 6007 (or a sequence with no more than 1, 2, 3, or 4 mutations, e.g., substitutions, additions, or deletions), a VHCDR2 amino acid sequence of SEQ ID NO: 6008 (or a sequence with no more than 1, 2, 3, or 4 mutations, e.g., substitutions, additions, or deletions), and/or a VHCDR3 amino acid sequence of SEQ ID NO: 6009 (or a sequence with no more than 1, 2, 3, or 4 mutations, e.g., substitutions, additions, or deletions). In some embodiments, the NKp30 antigen binding domain comprises a VH comprising a VHCDR1 amino acid sequence of SEQ ID NO: 6007, a VHCDR2 amino acid sequence of SEQ ID NO: 6008, and/or a VHCDR3 amino acid sequence of SEQ ID NO: 6009.

In some embodiments, the antigen binding domain that targets NKp30 comprises a VL comprising a light chain complementarity determining region 1 (VLCDR1) amino acid sequence of SEQ ID NO: 6070 (or a sequence with no more than 1, 2, 3, or 4 mutations, e.g., substitutions, additions, or deletions), a VLCDR2 amino acid sequence of SEQ ID NO: 6071 (or a sequence with no more than 1, 2, 3, or 4 mutations, e.g., substitutions, additions, or deletions), and/or a VLCDR3 amino acid sequence of SEQ ID NO: 6072 (or a sequence with no more than 1, 2, 3, or 4 mutations, e.g., substitutions, additions, or deletions). In some embodiments, the antigen binding domain that targets NKp30 comprises a VL comprising a VLCDR1 amino acid sequence of SEQ ID NO: 6070, a VLCDR2 amino acid sequence of SEQ ID NO: 6071, and a VLCDR3 amino acid sequence of SEQ ID NO: 6072.

In some embodiments, the antigen binding domain that targets NKp30 comprises a VH comprising a heavy chain complementarity determining region 1 (VHCDR1) amino acid sequence of SEQ ID NO: 6007 (or a sequence with no more than 1, 2, 3, or 4 mutations, e.g., substitutions, additions, or deletions), a VHCDR2 amino acid sequence of SEQ ID NO: 6008 (or a sequence with no more than 1, 2, 3, or 4 mutations, e.g., substitutions, additions, or deletions), and/or a VHCDR3 amino acid sequence of SEQ ID NO: 6009 (or a sequence with no more than 1, 2, 3, or 4 mutations, e.g., substitutions, additions, or deletions), and a VL comprising a light chain complementarity determining region 1 (VLCDR1) amino acid sequence of SEQ ID NO: 6070 (or a sequence with no more than 1, 2, 3, or 4 mutations, e.g., substitutions, additions, or deletions), a VLCDR2 amino acid sequence of SEQ ID NO: 6071 (or a sequence with no more than 1, 2, 3, or 4 mutations, e.g., substitutions, additions, or deletions), and/or a VLCDR3 amino acid sequence of SEQ ID NO: 6072 (or a sequence with no more than 1, 2, 3, or 4 mutations, e.g., substitutions, additions, or deletions). In some embodiments, the NKp30 antigen binding domain comprises a VH comprising a VHCDR1 amino acid sequence of SEQ ID NO: 6007, a VHCDR2 amino acid sequence of SEQ ID NO: 6008, and/or a VHCDR3 amino acid sequence of SEQ ID NO: 6009, and a VL comprising a VLCDR1 amino acid sequence of SEQ ID NO: 6070, a VLCDR2 amino acid sequence of SEQ ID NO: 6071, and a VLCDR3 amino acid sequence of SEQ ID NO: 6072.

In some embodiments, the antigen binding domain that targets NKp30 comprises a VH comprising a heavy chain framework region 1 (VHFWR1) amino acid sequence of SEQ ID NO: 6003, a VHFWR2 amino acid sequence of SEQ ID NO: 6004, a VHFWR3 amino acid sequence of SEQ ID NO: 6005, and/or a VHFWR4 amino acid sequence of SEQ ID NO: 6006.

In some embodiments, the antigen binding domain that targets NKp30 comprises a VL comprising a light chain framework region 1 (VLFWR1) amino acid sequence of SEQ ID NO: 6066, a VLFWR2 amino acid sequence of SEQ ID NO: 6067, a VLFWR3 amino acid sequence of SEQ ID NO: 7292, and/or a VLFWR4 amino acid sequence of SEQ ID NO: 6069.

In some embodiments, the antigen binding domain that targets NKp30 comprises a VH comprising a heavy chain framework region 1 (VHFWR1) amino acid sequence of SEQ ID NO: 6003, a VHFWR2 amino acid sequence of SEQ ID NO: 6004, a VHFWR3 amino acid sequence of SEQ ID NO: 6005, and/or a VHFWR4 amino acid sequence of SEQ ID NO: 6006, and a VL comprising a light chain framework region 1 (VLFWR1) amino acid sequence of SEQ ID NO: 6066, a VLFWR2 amino acid sequence of SEQ ID NO: 6067, a VLFWR3 amino acid sequence of SEQ ID NO: 7292, and/or a VLFWR4 amino acid sequence of SEQ ID NO: 6069.

In some embodiments, the antigen binding domain that targets NKp30 comprises a VH comprising a VHFWR1 amino acid sequence of SEQ ID NO: 6003 (or a sequence with no more than 1, 2, 3, 4, 5, or 6 mutations, e.g., substitutions, additions, or deletions, therefrom), a VHFWR2 amino acid sequence of SEQ ID NO: 6004 (or a sequence with no more than 1, 2, 3, 4, 5, or 6 mutations, e.g., substitutions, additions, or deletions, therefrom), a VHFWR3 amino acid sequence of SEQ ID NO: 6005 (or a sequence with no more than 1, 2, 3, 4, 5, 6, 7, 8, 9, 10, or 11 mutations, e.g., substitutions, additions, or deletions), and/or a VHFWR4 amino acid sequence of SEQ ID NO: 6006.

In some embodiments, the antigen binding domain that targets NKp30 comprises a VL comprising a VLFWR1 amino acid sequence of SEQ ID NO: 6066 (or a sequence with no more than 1, 2, or 3 mutations, e.g., substitutions, additions, or deletions), a VLFWR2 amino acid sequence of SEQ ID NO: 6067 (or a sequence with no more than 1 mutation, e.g., substitution, addition, or deletion), a VLFWR3 amino acid sequence of SEQ ID NO: 7292 (or a sequence with no more than 1 mutation, e.g., substitution, addition, or deletion), and/or a VLFWR4 amino acid sequence of SEQ ID NO: 6069.

In some embodiments, the antigen binding domain that targets NKp30 comprises a VH comprising a VHFWR1 amino acid sequence of SEQ ID NO: 6003 (or a sequence with no more than 1, 2, 3, 4, 5, or 6 mutations, e.g., substitutions, additions, or deletions, therefrom), a VHFWR2 amino acid sequence of SEQ ID NO: 6004 (or a sequence with no more than 1, 2, 3, 4, 5, or 6 mutations, e.g., substitutions, additions, or deletions, therefrom), a VHFWR3 amino acid sequence of SEQ ID NO: 6005 (or a sequence with no more than 1, 2, 3, 4, 5, 6, 7, 8, 9, 10, or 11 mutations, e.g., substitutions, additions, or deletions), and/or a VHFWR4 amino acid sequence of SEQ ID NO: 6006, and a VL comprising a VLFWR1 amino acid sequence of SEQ ID NO: 6066 (or a sequence with no more than 1, 2, or 3 mutations, e.g., substitutions, additions, or deletions), a VLFWR2 amino acid sequence of SEQ ID NO: 6067 (or a sequence with no more than 1 mutation, e.g., substitution, addition, or deletion), a VLFWR3 amino acid sequence of SEQ ID NO: 7292 (or a sequence with no more than 1 mutation, e.g., substitution, addition, or deletion), and/or a VLFWR4 amino acid sequence of SEQ ID NO: 6069.

In some embodiments, the antigen binding domain that targets NKp30 comprises a VH comprising a heavy chain framework region 1 (VHFWR1) amino acid sequence of SEQ ID NO: 6010, a VHFWR2 amino acid sequence of SEQ ID NO: 6011, a VHFWR3 amino acid sequence of SEQ ID NO: 6012, and/or a VHFWR4 amino acid sequence of SEQ ID NO: 6013.

In some embodiments, the antigen binding domain that targets NKp30 comprises a VL comprising a light chain framework region 1 (VLFWR1) amino acid sequence of SEQ ID NO: 6073, a VLFWR2 amino acid sequence of SEQ ID NO: 6074, a VLFWR3 amino acid sequence of SEQ ID NO: 6075, and/or a VLFWR4 amino acid sequence of SEQ ID NO: 6076.

In some embodiments, the antigen binding domain that targets NKp30 comprises a VH comprising a heavy chain framework region 1 (VHFWR1) amino acid sequence of SEQ ID NO: 6010, a VHFWR2 amino acid sequence of SEQ ID NO: 6011, a VHFWR3 amino acid sequence of SEQ ID NO: 6012, and/or a VHFWR4 amino acid sequence of SEQ ID NO: 6013, and a VL comprising a light chain framework region 1 (VLFWR1) amino acid sequence of SEQ ID NO: 6073, a VLFWR2 amino acid sequence of SEQ ID NO: 6074, a VLFWR3 amino acid sequence of SEQ ID NO: 6075, and/or a VLFWR4 amino acid sequence of SEQ ID NO: 6076.

In some embodiments, the antigen binding domain that targets NKp30 comprises a VH comprising a VHFWR1 amino acid sequence of SEQ ID NO: 6010 (or a sequence with no more than 1, 2, 3, 4, 5, or 6 mutations, e.g., substitutions, additions, or deletions, therefrom), a VHFWR2 amino acid sequence of SEQ ID NO: 6011 (or a sequence with no more than 1, 2, 3, 4, 5, or 6 mutations, e.g., substitutions, additions, or deletions, therefrom), a VHFWR3 amino acid sequence of SEQ ID NO: 6012 (or a sequence with no more than 1, 2, 3, 4, 5, 6, 7, 8, 9, 10, or 11 mutations, e.g., substitutions, additions, or deletions), and/or a VHFWR4 amino acid sequence of SEQ ID NO: 6013.

In some embodiments, the antigen binding domain that targets NKp30 comprises a VL comprising a VLFWR1 amino acid sequence of SEQ ID NO: 6073 (or a sequence with no more than 1, 2, or 3 mutations, e.g., substitutions, additions, or deletions), a VLFWR2 amino acid sequence of SEQ ID NO: 6074 (or a sequence with no more than 1 mutation, e.g., substitution, addition, or deletion), a VLFWR3 amino acid sequence of SEQ ID NO: 6075 (or a sequence with no more than 1 mutation, e.g., substitution, addition, or deletion), and/or a VLFWR4 amino acid sequence of SEQ ID NO: 6076.

In some embodiments, the antigen binding domain that targets NKp30 comprises a VH comprising a VHFWR1 amino acid sequence of SEQ ID NO: 6010 (or a sequence with no more than 1, 2, 3, 4, 5, or 6 mutations, e.g., substitutions, additions, or deletions, therefrom), a VHFWR2 amino acid sequence of SEQ ID NO: 6011 (or a sequence with no more than 1, 2, 3, 4, 5, or 6 mutations, e.g., substitutions, additions, or deletions, therefrom), a VHFWR3 amino acid sequence of SEQ ID NO: 6012 (or a sequence with no more than 1, 2, 3, 4, 5, 6, 7, 8, 9, 10, or 11 mutations, e.g., substitutions, additions, or deletions), and/or a VHFWR4 amino acid sequence of SEQ ID NO: 6013, and a VL comprising a VLFWR1 amino acid sequence of SEQ ID NO: 6073 (or a sequence with no more than 1, 2, or 3 mutations, e.g., substitutions, additions, or deletions), a VLFWR2 amino acid sequence of SEQ ID NO: 6074 (or a sequence with no more than 1 mutation, e.g., substitution, addition, or deletion), a VLFWR3 amino acid sequence of SEQ ID NO: 6075 (or a sequence with no more than 1 mutation, e.g., substitution, addition, or deletion), and/or a VLFWR4 amino acid sequence of SEQ ID NO: 6076.

In some embodiments, the antigen binding domain that targets NKp30 comprises a VH comprising a heavy chain framework region 1 (VHFWR1) amino acid sequence of SEQ ID NO: 6014, a VHFWR2 amino acid sequence of SEQ ID NO: 6015, a VHFWR3 amino acid sequence of SEQ ID NO: 6016, and/or a VHFWR4 amino acid sequence of SEQ ID NO: 6017.

In some embodiments, the antigen binding domain that targets NKp30 comprises a VH comprising a VHFWR1 amino acid sequence of SEQ ID NO: 6014 (or a sequence with no more than 1, 2, 3, 4, 5, or 6 mutations, e.g., substitutions, additions, or deletions, therefrom), a VHFWR2 amino acid sequence of SEQ ID NO: 6015 (or a sequence with no more than 1, 2, 3, 4, 5, or 6 mutations, e.g., substitutions, additions, or deletions, therefrom), a VHFWR3 amino acid sequence of SEQ ID NO: 6016 (or a sequence with no more than 1, 2, 3, 4, 5, 6, 7, 8, 9, 10, or 11 mutations, e.g., substitutions, additions, or deletions), and/or a VHFWR4 amino acid sequence of SEQ ID NO: 6017.

In some embodiments, the antigen binding domain that targets NKp30 comprises a VL comprising a light chain framework region 1 (VLFWR1) amino acid sequence of SEQ ID NO: 6077, a VLFWR2 amino acid sequence of SEQ ID NO: 6078, a VLFWR3 amino acid sequence of SEQ ID NO: 6079, and/or a VLFWR4 amino acid sequence of SEQ ID NO: 6080.

In some embodiments, the antigen binding domain that targets NKp30 comprises a VL comprising a VLFWR1 amino acid sequence of SEQ ID NO: 6077 (or a sequence with no more than 1, 2, or 3 mutations, e.g., substitutions, additions, or deletions), a VLFWR2 amino acid sequence of SEQ ID NO: 6078 (or a sequence with no more than 1 mutation, e.g., substitution, addition, or deletion), a VLFWR3 amino acid sequence of SEQ ID NO: 6079 (or a sequence with no more than 1 mutation, e.g., substitution, addition, or deletion), and/or a VLFWR4 amino acid sequence of SEQ ID NO: 6080.

In some embodiments, the antigen binding domain that targets NKp30 comprises a VH comprising a heavy chain framework region 1 (VHFWR1) amino acid sequence of SEQ ID NO: 6018, a VHFWR2 amino acid sequence of SEQ ID NO: 6019, a VHFWR3 amino acid sequence of SEQ ID NO: 6020, and/or a VHFWR4 amino acid sequence of SEQ ID NO: 6021.

In some embodiments, the antigen binding domain that targets NKp30 comprises a VH comprising a VHFWR1 amino acid sequence of SEQ ID NO: 6018 (or a sequence with no more than 1, 2, 3, 4, 5, or 6 mutations, e.g., substitutions, additions, or deletions, therefrom), a VHFWR2 amino acid sequence of SEQ ID NO: 6019 (or a sequence with no more than 1, 2, 3, 4, 5, or 6 mutations, e.g., substitutions, additions, or deletions, therefrom), a VHFWR3 amino acid sequence of SEQ ID NO: 6020 (or a sequence with no more than 1, 2, 3, 4, 5, 6, 7, 8, 9, 10, or 11 mutations, e.g., substitutions, additions, or deletions), and/or a VHFWR4 amino acid sequence of SEQ ID NO: 6021.

In some embodiments, the antigen binding domain that targets NKp30 comprises a VL comprising a light chain framework region 1 (VLFWR1) amino acid sequence of SEQ ID NO: 6081, a VLFWR2 amino acid sequence of SEQ ID NO: 6082, a VLFWR3 amino acid sequence of SEQ ID NO: 6083, and/or a VLFWR4 amino acid sequence of SEQ ID NO: 6084.

In some embodiments, the antigen binding domain that targets NKp30 comprises a VL comprising a VLFWR1 amino acid sequence of SEQ ID NO: 6081 (or a sequence with no more than 1, 2, or 3 mutations, e.g., substitutions, additions, or deletions), a VLFWR2 amino acid sequence of SEQ ID NO: 6082 (or a sequence with no more than 1 mutation, e.g., substitution, addition, or deletion), a VLFWR3 amino acid sequence of SEQ ID NO: 6083 (or a sequence with no more than 1 mutation, e.g., substitution, addition, or deletion), and/or a VLFWR4 amino acid sequence of SEQ ID NO: 6084.

In some embodiments, the antigen binding domain that targets NKp30 comprises a VH comprising a heavy chain framework region 1 (VHFWR1) amino acid sequence of SEQ ID NO: 6022, a VHFWR2 amino acid sequence of SEQ ID NO: 6023, a VHFWR3 amino acid sequence of SEQ ID NO: 6024, and/or a VHFWR4 amino acid sequence of SEQ ID NO: 6025.

In some embodiments, the antigen binding domain that targets NKp30 comprises a VH comprising a VHFWR1 amino acid sequence of SEQ ID NO: 6022 (or a sequence with no more than 1, 2, 3, 4, 5, or 6 mutations, e.g., substitutions, additions, or deletions, therefrom), a VHFWR2 amino acid sequence of SEQ ID NO: 6023 (or a sequence with no more than 1, 2, 3, 4, 5, or 6 mutations, e.g., substitutions, additions, or deletions, therefrom), a VHFWR3 amino acid sequence of SEQ ID NO: 6024 (or a sequence with no more than 1, 2, 3, 4, 5, 6, 7, 8, 9, 10, or 11 mutations, e.g., substitutions, additions, or deletions), and/or a VHFWR4 amino acid sequence of SEQ ID NO: 6025.

In some embodiments, the antigen binding domain that targets NKp30 comprises a VL comprising a light chain framework region 1 (VLFWR1) amino acid sequence of SEQ ID NO: 6085, a VLFWR2 amino acid sequence of SEQ ID NO: 6086, a VLFWR3 amino acid sequence of SEQ ID NO: 6087, and/or a VLFWR4 amino acid sequence of SEQ ID NO: 6088.

In some embodiments, the antigen binding domain that targets NKp30 comprises a VL comprising a VLFWR1 amino acid sequence of SEQ ID NO: 6085 (or a sequence with no more than 1, 2, or 3 mutations, e.g., substitutions, additions, or deletions), a VLFWR2 amino acid sequence of SEQ ID NO: 6086 (or a sequence with no more than 1 mutation, e.g., substitution, addition, or deletion), a VLFWR3 amino acid sequence of SEQ ID NO: 6087 (or a sequence with no more than 1 mutation, e.g., substitution, addition, or deletion), and/or a VLFWR4 amino acid sequence of SEQ ID NO: 6088.

In some embodiments, the antigen binding domain that targets NKp30 comprises a VH comprising a heavy chain framework region 1 (VHFWR1) amino acid sequence of SEQ ID NO: 6026, a VHFWR2 amino acid sequence of SEQ ID NO: 6027, a VHFWR3 amino acid sequence of SEQ ID NO: 6028, and/or a VHFWR4 amino acid sequence of SEQ ID NO: 6029.

In some embodiments, the antigen binding domain that targets NKp30 comprises a VH comprising a VHFWR1 amino acid sequence of SEQ ID NO: 6026 (or a sequence with no more than 1, 2, 3, 4, 5, or 6 mutations, e.g., substitutions, additions, or deletions, therefrom), a VHFWR2 amino acid sequence of SEQ ID NO: 6027 (or a sequence with no more than 1, 2, 3, 4, 5, or 6 mutations, e.g., substitutions, additions, or deletions, therefrom), a VHFWR3 amino acid sequence of SEQ ID NO: 6028 (or a sequence with no more than 1, 2, 3, 4, 5, 6, 7, 8, 9, 10, or 11 mutations, e.g., substitutions, additions, or deletions), and/or a VHFWR4 amino acid sequence of SEQ ID NO: 6029.

In some embodiments, the antigen binding domain that targets NKp30 comprises a VL comprising a light chain framework region 1 (VLFWR1) amino acid sequence of SEQ ID NO: 6089, a VLFWR2 amino acid sequence of SEQ ID NO: 6090, a VLFWR3 amino acid sequence of SEQ ID NO: 6091, and/or a VLFWR4 amino acid sequence of SEQ ID NO: 6092.

In some embodiments, the antigen binding domain that targets NKp30 comprises a VL comprising a VLFWR1 amino acid sequence of SEQ ID NO: 6089 (or a sequence with no more than 1, 2, or 3 mutations, e.g., substitutions, additions, or deletions), a VLFWR2 amino acid sequence of SEQ ID NO: 6090 (or a sequence with no more than 1 mutation, e.g., substitution, addition, or deletion), a VLFWR3 amino acid sequence of SEQ ID NO: 6091 (or a sequence with no more than 1 mutation, e.g., substitution, addition, or deletion), and/or a VLFWR4 amino acid sequence of SEQ ID NO: 6092.

In some embodiments, the antigen binding domain that targets NKp30 comprises a VH comprising a heavy chain framework region 1 (VHFWR1) amino acid sequence of SEQ ID NO: 6030, a VHFWR2 amino acid sequence of SEQ ID NO: 6032, a VHFWR3 amino acid sequence of SEQ ID NO: 6033, and/or a VHFWR4 amino acid sequence of SEQ ID NO: 6034.

In some embodiments, the antigen binding domain that targets NKp30 comprises a VH comprising a VHFWR1 amino acid sequence of SEQ ID NO: 6030 (or a sequence with no more than 1, 2, 3, 4, 5, or 6 mutations, e.g., substitutions, additions, or deletions, therefrom), a VHFWR2 amino acid sequence of SEQ ID NO: 6032 (or a sequence with no more than 1, 2, 3, 4, 5, or 6 mutations, e.g., substitutions, additions, or deletions, therefrom), a VHFWR3 amino acid sequence of SEQ ID NO: 6033 (or a sequence with no more than 1, 2, 3, 4, 5, 6, 7, 8, 9, 10, or 11 mutations, e.g., substitutions, additions, or deletions), and/or a VHFWR4 amino acid sequence of SEQ ID NO: 6034.

In some embodiments, the antigen binding domain that targets NKp30 comprises a VL comprising a light chain framework region 1 (VLFWR1) amino acid sequence of SEQ ID NO: 6093, a VLFWR2 amino acid sequence of SEQ ID NO: 6094, a VLFWR3 amino acid sequence of SEQ ID NO: 6095, and/or a VLFWR4 amino acid sequence of SEQ ID NO: 6096.

In some embodiments, the antigen binding domain that targets NKp30 comprises a VL comprising a VLFWR1 amino acid sequence of SEQ ID NO: 6093 (or a sequence with no more than 1, 2, or 3 mutations, e.g., substitutions, additions, or deletions), a VLFWR2 amino acid sequence of SEQ ID NO: 6094 (or a sequence with no more than 1 mutation, e.g., substitution, addition, or deletion), a VLFWR3 amino acid sequence of SEQ ID NO: 6095 (or a sequence with no more than 1 mutation, e.g., substitution, addition, or deletion), and/or a VLFWR4 amino acid sequence of SEQ ID NO: 6096.

In some embodiments, the antigen binding domain that targets NKp30 comprises a VH comprising a heavy chain framework region 1 (VHFWR1) amino acid sequence of SEQ ID NO: 6035, a VHFWR2 amino acid sequence of SEQ ID NO: 6036, a VHFWR3 amino acid sequence of SEQ ID NO: 6037, and/or a VHFWR4 amino acid sequence of SEQ ID NO: 6038.

In some embodiments, the antigen binding domain that targets NKp30 comprises a VH comprising a VHFWR1 amino acid sequence of SEQ ID NO: 6035 (or a sequence with no more than 1, 2, 3, 4, 5, or 6 mutations, e.g., substitutions, additions, or deletions, therefrom), a VHFWR2 amino acid sequence of SEQ ID NO: 6036 (or a sequence with no more than 1, 2, 3, 4, 5, or 6 mutations, e.g., substitutions, additions, or deletions, therefrom), a VHFWR3 amino acid sequence of SEQ ID NO: 6037 (or a sequence with no more than 1, 2, 3, 4, 5, 6, 7, 8, 9, 10, or 11 mutations, e.g., substitutions, additions, or deletions), and/or a VHFWR4 amino acid sequence of SEQ ID NO: 6038.

In some embodiments, the antigen binding domain that targets NKp30 comprises a VH comprising a heavy chain framework region 1 (VHFWR1) amino acid sequence of SEQ ID NO: 6039, a VHFWR2 amino acid sequence of SEQ ID NO: 6040, a VHFWR3 amino acid sequence of SEQ ID NO: 6041, and/or a VHFWR4 amino acid sequence of SEQ ID NO: 6042.

In some embodiments, the antigen binding domain that targets NKp30 comprises a VH comprising a VHFWR1 amino acid sequence of SEQ ID NO: 6039 (or a sequence with no more than 1, 2, 3, 4, 5, or 6 mutations, e.g., substitutions, additions, or deletions, therefrom), a VHFWR2 amino acid sequence of SEQ ID NO: 6040 (or a sequence with no more than 1, 2, 3, 4, 5, or 6 mutations, e.g., substitutions, additions, or deletions, therefrom), a VHFWR3 amino acid sequence of SEQ ID NO: 6041 (or a sequence with no more than 1, 2, 3, 4, 5, 6, 7, 8, 9, 10, or 11 mutations, e.g., substitutions, additions, or deletions), and/or a VHFWR4 amino acid sequence of SEQ ID NO: 6042.

In some embodiments, the antigen binding domain that targets NKp30 comprises a VL comprising a light chain framework region 1 (VLFWR1) amino acid sequence of SEQ ID NO: 6097, a VLFWR2 amino acid sequence of SEQ ID NO: 6098, a VLFWR3 amino acid sequence of SEQ ID NO: 6099, and/or a VLFWR4 amino acid sequence of SEQ ID NO: 6100.

In some embodiments, the antigen binding domain that targets NKp30 comprises a VL comprising a VLFWR1 amino acid sequence of SEQ ID NO: 6097 (or a sequence with no more than 1, 2, or 3 mutations, e.g., substitutions, additions, or deletions), a VLFWR2 amino acid sequence of SEQ ID NO: 6098 (or a sequence with no more than 1 mutation, e.g., substitution, addition, or deletion), a VLFWR3 amino acid sequence of SEQ ID NO: 6099 (or a sequence with no more than 1 mutation, e.g., substitution, addition, or deletion), and/or a VLFWR4 amino acid sequence of SEQ ID NO: 6100.

In some embodiments, the antigen binding domain that targets NKp30 comprises a VH comprising a heavy chain framework region 1 (VHFWR1) amino acid sequence of SEQ ID NO: 6043, a VHFWR2 amino acid sequence of SEQ ID NO: 6044, a VHFWR3 amino acid sequence of SEQ ID NO: 6045, and/or a VHFWR4 amino acid sequence of SEQ ID NO: 6046.

In some embodiments, the antigen binding domain that targets NKp30 comprises a VH comprising a VHFWR1 amino acid sequence of SEQ ID NO: 6043 (or a sequence with no more than 1, 2, 3, 4, 5, or 6 mutations, e.g., substitutions, additions, or deletions, therefrom), a VHFWR2 amino acid sequence of SEQ ID NO: 6044 (or a sequence with no more than 1, 2, 3, 4, 5, or 6 mutations, e.g., substitutions, additions, or deletions, therefrom), a VHFWR3 amino acid sequence of SEQ ID NO: 6045 (or a sequence with no more than 1, 2, 3, 4, 5, 6, 7, 8, 9, 10, or 11 mutations, e.g., substitutions, additions, or deletions), and/or a VHFWR4 amino acid sequence of SEQ ID NO: 6046.

In some embodiments, the antigen binding domain that targets NKp30 comprises a VL comprising a light chain framework region 1 (VLFWR1) amino acid sequence of SEQ ID NO: 6101, a VLFWR2 amino acid sequence of SEQ ID NO: 6102, a VLFWR3 amino acid sequence of SEQ ID NO: 6103, and/or a VLFWR4 amino acid sequence of SEQ ID NO: 6104.

In some embodiments, the antigen binding domain that targets NKp30 comprises a VL comprising a VLFWR1 amino acid sequence of SEQ ID NO: 6101 (or a sequence with no more than 1, 2, or 3 mutations, e.g., substitutions, additions, or deletions), a VLFWR2 amino acid sequence of SEQ ID NO: 6102 (or a sequence with no more than 1 mutation, e.g., substitution, addition, or deletion), a VLFWR3 amino acid sequence of SEQ ID NO: 6103 (or a sequence with no more than 1 mutation, e.g., substitution, addition, or deletion), and/or a VLFWR4 amino acid sequence of SEQ ID NO: 6104.

In some embodiments, the antigen binding domain that targets NKp30 comprises a VH comprising a heavy chain framework region 1 (VHFWR1) amino acid sequence of SEQ ID NO: 6047, a VHFWR2 amino acid sequence of SEQ ID NO: 6048, a VHFWR3 amino acid sequence of SEQ ID NO: 6049, and/or a VHFWR4 amino acid sequence of SEQ ID NO: 6050.

In some embodiments, the antigen binding domain that targets NKp30 comprises a VH comprising a VHFWR1 amino acid sequence of SEQ ID NO: 6047 (or a sequence with no more than 1, 2, 3, 4, 5, or 6 mutations, e.g., substitutions, additions, or deletions, therefrom), a VHFWR2 amino acid sequence of SEQ ID NO: 6048 (or a sequence with no more than 1, 2, 3, 4, 5, or 6 mutations, e.g., substitutions, additions, or deletions, therefrom), a VHFWR3 amino acid sequence of SEQ ID NO: 6049 (or a sequence with no more than 1, 2, 3, 4, 5, 6, 7, 8, 9, 10, or 11 mutations, e.g., substitutions, additions, or deletions), and/or a VHFWR4 amino acid sequence of SEQ ID NO: 6050.

In some embodiments, the antigen binding domain that targets NKp30 comprises a VL comprising a light chain framework region 1 (VLFWR1) amino acid sequence of SEQ ID NO: 6105, a VLFWR2 amino acid sequence of SEQ ID NO: 6106, a VLFWR3 amino acid sequence of SEQ ID NO: 6107, and/or a VLFWR4 amino acid sequence of SEQ ID NO: 6108.

In some embodiments, the antigen binding domain that targets NKp30 comprises a VL comprising a VLFWR1 amino acid sequence of SEQ ID NO: 6105 (or a sequence with no more than 1, 2, or 3 mutations, e.g., substitutions, additions, or deletions), a VLFWR2 amino acid sequence of SEQ ID NO: 6106 (or a sequence with no more than 1 mutation, e.g., substitution, addition, or deletion), a VLFWR3 amino acid sequence of SEQ ID NO: 6107 (or a sequence with no more than 1 mutation, e.g., substitution, addition, or deletion), and/or a VLFWR4 amino acid sequence of SEQ ID NO: 6108.

In some embodiments, the antigen binding domain that targets NKp30 comprises a VH comprising a heavy chain framework region 1 (VHFWR1) amino acid sequence of SEQ ID NO: 6051, a VHFWR2 amino acid sequence of SEQ ID NO: 6052, a VHFWR3 amino acid sequence of SEQ ID NO: 6053, and/or a VHFWR4 amino acid sequence of SEQ ID NO: 6054.

In some embodiments, the antigen binding domain that targets NKp30 comprises a VH comprising a VHFWR1 amino acid sequence of SEQ ID NO: 6051 (or a sequence with no more than 1, 2, 3, 4, 5, or 6 mutations, e.g., substitutions, additions, or deletions, therefrom), a VHFWR2 amino acid sequence of SEQ ID NO: 6052 (or a sequence with no more than 1, 2, 3, 4, 5, or 6 mutations, e.g., substitutions, additions, or deletions, therefrom), a VHFWR3 amino acid sequence of SEQ ID NO: 6053 (or a sequence with no more than 1, 2, 3, 4, 5, 6, 7, 8, 9, 10, or 11 mutations, e.g., substitutions, additions, or deletions), and/or a VHFWR4 amino acid sequence of SEQ ID NO: 6054.

In some embodiments, the antigen binding domain that targets NKp30 comprises a VL comprising a light chain framework region 1 (VLFWR1) amino acid sequence of SEQ ID NO: 6109, a VLFWR2 amino acid sequence of SEQ ID NO: 6110, a VLFWR3 amino acid sequence of SEQ ID NO: 6111, and/or a VLFWR4 amino acid sequence of SEQ ID NO: 6112.

In some embodiments, the antigen binding domain that targets NKp30 comprises a VL comprising a VLFWR1 amino acid sequence of SEQ ID NO: 6109 (or a sequence with no more than 1, 2, or 3 mutations, e.g., substitutions, additions, or deletions), a VLFWR2 amino acid sequence of SEQ ID NO: 6110 (or a sequence with no more than 1 mutation, e.g., substitution, addition, or deletion), a VLFWR3 amino acid sequence of SEQ ID NO: 6111 (or a sequence with no more than 1 mutation, e.g., substitution, addition, or deletion), and/or a VLFWR4 amino acid sequence of SEQ ID NO: 6112.

In some embodiments, the antigen binding domain that targets NKp30 comprises a VH comprising a heavy chain framework region 1 (VHFWR1) amino acid sequence of SEQ ID NO: 6055, a VHFWR2 amino acid sequence of SEQ ID NO: 6056, a VHFWR3 amino acid sequence of SEQ ID NO: 6057, and/or a VHFWR4 amino acid sequence of SEQ ID NO: 6058.

In some embodiments, the antigen binding domain that targets NKp30 comprises a VH comprising a VHFWR1 amino acid sequence of SEQ ID NO: 6055 (or a sequence with no more than 1, 2, 3, 4, 5, or 6 mutations, e.g., substitutions, additions, or deletions, therefrom), a VHFWR2 amino acid sequence of SEQ ID NO: 6056 (or a sequence with no more than 1, 2, 3, 4, 5, or 6 mutations, e.g., substitutions, additions, or deletions, therefrom), a VHFWR3 amino acid sequence of SEQ ID NO: 6057 (or a sequence with no more than 1, 2, 3, 4, 5, 6, 7, 8, 9, 10, or 11 mutations, e.g., substitutions, additions, or deletions), and/or a VHFWR4 amino acid sequence of SEQ ID NO: 6058.

In some embodiments, the antigen binding domain that targets NKp30 comprises a VL comprising a light chain framework region 1 (VLFWR1) amino acid sequence of SEQ ID NO: 6113, a VLFWR2 amino acid sequence of SEQ ID NO: 6114, a VLFWR3 amino acid sequence of SEQ ID NO: 6115, and/or a VLFWR4 amino acid sequence of SEQ ID NO: 6116.

In some embodiments, the antigen binding domain that targets NKp30 comprises a VL comprising a VLFWR1 amino acid sequence of SEQ ID NO: 6113 (or a sequence with no more than 1, 2, or 3 mutations, e.g., substitutions, additions, or deletions), a VLFWR2 amino acid sequence of SEQ ID NO: 6114 (or a sequence with no more than 1 mutation, e.g., substitution, addition, or deletion), a VLFWR3 amino acid sequence of SEQ ID NO: 6115 (or a sequence with no more than 1 mutation, e.g., substitution, addition, or deletion), and/or a VLFWR4 amino acid sequence of SEQ ID NO: 6116.

In some embodiments, the antigen binding domain that targets NKp30 comprises a VH comprising a heavy chain framework region 1 (VHFWR1) amino acid sequence of SEQ ID NO: 6059, a VHFWR2 amino acid sequence of SEQ ID NO: 6060, a VHFWR3 amino acid sequence of SEQ ID NO: 6061, and/or a VHFWR4 amino acid sequence of SEQ ID NO: 6062.

In some embodiments, the antigen binding domain that targets NKp30 comprises a VH comprising a VHFWR1 amino acid sequence of SEQ ID NO: 6059 (or a sequence with no more than 1, 2, 3, 4, 5, or 6 mutations, e.g., substitutions, additions, or deletions, therefrom), a VHFWR2 amino acid sequence of SEQ ID NO: 6060 (or a sequence with no more than 1, 2, 3, 4, 5, or 6 mutations, e.g., substitutions, additions, or deletions, therefrom), a VHFWR3 amino acid sequence of SEQ ID NO: 6061 (or a sequence with no more than 1, 2, 3, 4, 5, 6, 7, 8, 9, 10, or 11 mutations, e.g., substitutions, additions, or deletions), and/or a VHFWR4 amino acid sequence of SEQ ID NO: 6062.

In some embodiments, the antigen binding domain that targets NKp30 comprises a VL comprising a light chain framework region 1 (VLFWR1) amino acid sequence of SEQ ID NO: 6117, a VLFWR2 amino acid sequence of SEQ ID NO: 6118, a VLFWR3 amino acid sequence of SEQ ID NO: 6119, and/or a VLFWR4 amino acid sequence of SEQ ID NO: 6120.

In some embodiments, the antigen binding domain that targets NKp30 comprises a VL comprising a VLFWR1 amino acid sequence of SEQ ID NO: 6117 (or a sequence with no more than 1, 2, or 3 mutations, e.g., substitutions, additions, or deletions), a VLFWR2 amino acid sequence of SEQ ID NO: 6118 (or a sequence with no more than 1 mutation, e.g., substitution, addition, or deletion), a VLFWR3 amino acid sequence of SEQ ID NO: 6119 (or a sequence with no more than 1 mutation, e.g., substitution, addition, or deletion), and/or a VLFWR4 amino acid sequence of SEQ ID NO: 6120.

In some embodiments, the antigen binding domain that targets NKp30 comprises a VH comprising the amino acid sequence of SEQ ID NO: 6148 (or an amino acid sequence having at least about 77%, 80%, 85%, 90%, 95%, or 99% sequence identity to SEQ ID NO: 6148). In some embodiments, the antigen binding domain that targets NKp30 comprises a VH comprising the amino acid sequence of SEQ ID NO: 6149 (or an amino acid sequence having at least about 77%, 80%, 85%, 90%, 95%, or 99% sequence identity to SEQ ID NO: 6149). In some embodiments, the antigen binding domain that targets NKp30 comprises a VL comprising the amino acid sequence of SEQ ID NO: 6150 (or an amino acid sequence having at least about 93%, 95%, or 99% sequence identity to SEQ ID NO: 6150). In some embodiments, antigen binding domain that targets NKp30 comprises a VH comprising the amino acid sequence of SEQ ID NO: 6148. In some embodiments, antigen binding domain that targets NKp30 comprises a VH comprising the amino acid sequence of SEQ ID NO: 6149. In some embodiments, the antigen binding domain that targets NKp30 comprises a VL comprising the amino acid sequence of SEQ ID NO: 6150.

In some embodiments, the antigen binding domain that targets NKp30 comprises a VH comprising the amino acid sequence of SEQ ID NO: 6148, and a VL comprising the amino acid sequence of SEQ ID NO: 6150. In some embodiments, the antigen binding domain that targets NKp30 comprises a VH comprising the amino acid sequence of SEQ ID NO: 6149, and a VL comprising the amino acid sequence of SEQ ID NO: 6150.

In some embodiments, the antigen binding domain that targets NKp30 comprises a VH comprising the amino acid sequence of SEQ ID NO: 6151 (or an amino acid sequence having at least about 77%, 80%, 85%, 90%, 95%, or 99% sequence identity to SEQ ID NO: 6151). In some embodiments, the antigen binding domain that targets NKp30 comprises a VH comprising the amino acid sequence of SEQ ID NO: 6152 (or an amino acid sequence having at least about 77%, 80%, 85%, 90%, 95%, or 99% sequence identity to SEQ ID NO: 6152). In some embodiments, the antigen binding domain that targets NKp30 comprises a VL comprising the amino acid sequence of SEQ ID NO: 6153 (or an amino acid sequence having at least about 93%, 95%, or 99% sequence identity to SEQ ID NO: 6153). In some embodiments, antigen binding domain that targets NKp30 comprises a VH comprising the amino acid sequence of SEQ ID NO: 6151. In some embodiments, antigen binding domain that targets NKp30 comprises a VH comprising the amino acid sequence of SEQ ID NO: 6152. In some embodiments, the antigen binding domain that targets NKp30 comprises a VL comprising the amino acid sequence of SEQ ID NO: 6153.

In some embodiments, the antigen binding domain that targets NKp30 comprises a VH comprising the amino acid sequence of SEQ ID NO: 6151, and a VL comprising the amino acid sequence of SEQ ID NO: 6153. In some embodiments, the antigen binding domain that targets NKp30 comprises a VH comprising the amino acid sequence of SEQ ID NO: 6152, and a VL comprising the amino acid sequence of SEQ ID NO: 6153.

In some embodiments, the antigen binding domain that targets NKp30 comprises an scFv. In some embodiments, the scFv comprises an amino acid sequence selected from SEQ ID NOs: 6187-6190, or an amino acid sequence having at least about 93%, 95%, or 99% sequence identity thereto.

TABLE 7
Exemplary heavy chain CDRs and FWRs of NKp30-targeting antigen binding domains

| Ab ID | VHFWR1 | VHCDR1 | VHFWR2 | VHCDR2 | VHFWR3 | VHCDR3 | VHFWR4 |
|---|---|---|---|---|---|---|---|
| 9G1- | QIQLQES | TGGYH | WIRQFP | YIYSSGS | RISITRDTS | GNWHY | WGQGT |
| HC | GPGLVKP | WN | GKKLE | TSYNPSL | KNQFFLQ | FDF (SEQ | MVTVSS |
| | SQSLSLT | (SEQ ID | WMG | KS (SEQ | LNSVTTE | ID NO: | (SEQ ID |
| | CSVTGFSI | NO: | (SEQ ID | ID NO: | DTATYYC | 6002) | NO: |
| | N (SEQ ID | 6000) | NO: | 6001) | AR (SEQ | | 6006) |
| | NO: 6003) | | 6004) | | ID NO: | | |
| | | | | | 6005) | | |
| 15H6- | QIQLQES | TGGYH | WIRQFP | YIYSSGT | RISITRDTS | GNWHY | WGQGT |
| HC | GPGLVKP | WN | GKKLE | TRYNPS | KNQFFLQ | FDY | LVAVSS |
| | SQSLSLT | (SEQ ID | WMG | LKS | LNSVTPED | (SEQ ID | (SEQ ID |
| | CSVTGFSI | NO: | (SEQ ID | (SEQ ID | TATYYCT | NO: 6009) | NO: |
| | N (SEQ ID | 6007) | NO: | NO: 6008) | R (SEQ ID | | 6013) |
| | NO: 6010) | | 6011) | | NO: 6012) | | |
| 9G1- | QIQLQES | TGGYH | WIRQPA | YIYSSGS | RVTMSRD | GNWHY | WGQGT |
| HC_1 | GPGLVKP | WN | GKGLE | TSYNPSL | TSKNQFSL | FDF (SEQ | MVTVSS |
| | SETLSLT | (SEQ ID | WIG | KS (SEQ | KLSSVTA | ID NO: | (SEQ ID |
| | CTVSGFSI | NO: | (SEQ ID | ID NO: | ADTAVYY | 6002) | NO: |
| | N (SEQ ID | 6000) | NO: | 6001) | CAR (SEQ | | 6017) |
| | NO: 6014) | | 6015) | | ID NO: | | |
| | | | | | 6016) | | |
| 9G1- | QIQLQES | TGGYH | WIRQHP | YIYSSGS | LVTISRDT | GNWHY | WGQGT |
| HC_2 | GPGLVKP | WN | GKGLE | TSYNPSL | SKNQFSL | FDF (SEQ | MVTVSS |
| | SQTLSLT | (SEQ ID | WIG | KS (SEQ | KLSSVTA | ID NO: | (SEQ ID |
| | CTVSGFSI | NO: | (SEQ ID | ID NO: | ADTAVYY | 6002) | NO: |
| | N (SEQ ID | 6000) | NO: | 6001) | CAR (SEQ | | 6021) |
| | NO: 6018) | | 6019) | | ID NO: | | |
| 9G1- | EIQLLES | TGGYH | WVRQA | YIYSSGS | RFTISRDT | GNWHY | WGQGT |
| HC_3 | GGGLVQ | WN | PGKGLE | TSYNPSL | SKNTFYL | FDF (SEQ | MVTVSS |
| | PGGSLRL | (SEQ ID | WVG | KS (SEQ | QMNSLRA | ID NO: | (SEQ ID |
| | SCAVSGF | NO: | (SEQ ID | ID NO: | EDTAVYY | 6002) | NO: |
| | SIN (SEQ | 6000) | NO: | 6001) | CAR (SEQ | | 6025) |
| | ID NO: | | 6023) | | ID NO: | | |
| | 6022) | | | | 6024) | | |
| 9G1- | QIQLVQS | TGGYH | WVRQA | YIYSSGS | RVTITRDT | GNWHY | WGQGT |
| HC_4 | GAEVKK | WN | PGQGLE | TSYNPSL | STNTFYM | FDF (SEQ | MVTVSS |
| | PGSSVKV | (SEQ ID | WMG | KS (SEQ | ELSSLRSE | ID NO: | (SEQ ID |
| | SCKVSGF | NO: | (SEQ ID | ID NO: | DTAVYYC | 6002) | NO: |
| | SIN (SEQ | 6000) | NO | 6001) | AR (SEQ | | 6029) |
| | ID NO: | | 6027) | | ID NO: | | |
| | 6026) | | | | 6028) | | |
| 9G1- | EIQLVES | TGGYH | WVRQA | YIYSSGS | RFTISRDT | GNWHY | WGQGT |
| HC_5 | GGGLVQ | WN | PGKGLE | TSYNPSL | AKNSFYL | FDF (SEQ | MVTVSS |
| | PGGSLRL | (SEQ ID | WVG | KS (SEQ | QMNSLRA | ID NO: | (SEQ ID |
| | SCAVSGF | NO: | (SEQ ID | ID NO: | EDTAVYY | 6002) | NO |
| | SIN (SEQ | 6000) | NO: | 6001) | CAR (SEQ | | 6034) |
| | ID NO: | | 6032) | | ID NO: | | |
| | 6030) | | | | 6033) | | |
| 9G1- | QIQLVQS | TGGYH | WVRQA | YIYSSGS | RVTMTRD | GNWHY | WGQGT |
| HC_6 | GAEVKK | WN | PGQGLE | TSYNPSL | TSTNTFY | FDF (SEQ | MVTVSS |
| | PGASVKV | (SEQ ID | WMG | KS (SEQ | MELSSLRS | ID NO: | (SEQ ID |
| | SCKVSGF | NO: | (SEQ ID | ID NO: | EDTAVYY | 6002) | NO: |
| | SIN (SEQ | 6000) | NO: | 6001) | CAR (SEQ | | 6038) |
| | ID NO: | | 6036) | | ID NO: | | |
| | 6035) | | | | 6037) | | |
| 15H6- | QIQLQES | TGGYH | WIRQHP | YIYSSGT | LVTISRDT | GNWHY | WGQGT |
| HC_1 | GPGLVKP | WN | GKGLE | TRYNPS | SKNQFSL | FDY | LVTVSS |
| | SQTLSLT | (SEQ ID | WIG | LKS | KLSSVTA | (SEQ ID | (SEQ ID |
| | CTVSGFSI | NO: | (SEQ ID | (SEQ ID | ADTAVYY | NO: 6009) | NO: |
| | N (SEQ ID | 6007) | NO: | NO: 6008) | CAR (SEQ | | 6042) |
| | NO: 6039) | | 6040) | | ID NO: | | |
| | | | | | 6041) | | |
| 15H6- | QIQLQES | TGGYH | WIRQPA | YIYSSGT | RVTMSRD | GNWHY | WGQGT |
| HC_2 | GPGLVKP | WN | GKGLE | TRYNPS | TSKNQFSL | FDY | LVTVSS |
| | SETLSLT | (SEQ ID | WIG | LKS | KLSSVTA | (SEQ ID | (SEQ ID |
| | CTVSGFSI | NO: | (SEQ ID | (SEQ ID | ADTAVYY | NO: 6009) | NO |
| | N (SEQ ID | 6007) | NO: | NO: 6008) | CAR (SEQ | | 6046) |
| | NO: 6043) | | 6044) | | ID NO: | | |
| | | | | | 6045) | | |
| 15H6- | EIQLLES | TGGYH | WVRQA | YIYSSGT | RFTISRDT | GNWHY | WGQGT |
| HC_3 | GGGLVQ | WN | PGKGLE | TRYNPS | SKNTFYL | FDY | LVTVSS |
| | PGGSLRL | (SEQ ID | WVG | LKS | QMNSLRA | (SEQ ID | (SEQ ID |
| | SCAVSGF | NO: | (SEQ ID | (SEQ ID | EDTAVYY | NO: 6009) | NO: |
| | SIN (SEQ | 6007) | NO | NO: 6008) | CAR (SEQ | | 6050) |
| | ID NO: | | 6048) | | ID NO: | | |
| | 6047) | | | | 6049) | | |
| 15H6- | QIQLVES | TGGYH | WIRQAP | YIYSSGT | RFTISRDT | GNWHY | WGQGT |
| HC_4 | GGGLVK | WN | GKGLE | TRYNPS | AKNSFYL | FDY | LVTVSS |
| | PGGSLRL | (SEQ ID | WVG | LKS | QMNSLRA | (SEQ ID | (SEQ ID |
| | SCAVSGF | NO: | (SEQ ID | (SEQ ID | EDTAVYY | NO: 6009) | NO: |
| | SIN (SEQ | 6007) | NO: | NO: 6008) | CAR (SEQ | | 6054) |
| | ID NO: | | 6052) | | ID NO: | | |
| | 6051) | | | | 6053) | | |
| 15H6- | QIQLVQS | TGGYH | WVRQA | YIYSSGT | RVTMTRD | GNWHY | WGQGT |
| HC_5 | GAEVKK | WN | PGQGLE | TRYNPS | TSTNTFY | FDY | LVTVSS |
| | PGASVKV | (SEQ ID | WMG | LKS | MELSSLRS | (SEQ ID | (SEQ ID |
| | SCKVSGF | NO: | (SEQ ID | (SEQ ID | EDTAVYY | NO: 6009) | NO: |
| | SIN (SEQ | 6007) | NO: | NO: 6008) | CAR (SEQ | | 6058) |
| | ID NO: | | 6056) | | ID NO: | | |
| | 6055) | | | | 6057) | | |
| 15H6- | EIQLVQS | TGGYH | WVQQA | YIYSSGT | RVTITRDT | GNWHY | WGQGT |
| HC_6 | GAEVKK | WN | PGKGLE | TRYNPS | STNTFYM | FDY | LVTVSS |
| | PGATVKI | (SEQ ID | WMG | LKS | ELSSLRSE | (SEQ ID | (SEQ ID |
| | SCKVSGF | NO: | (SEQ ID | (SEQ ID | DTAVYYC | NO: 6009) | NO: |
| | SIN (SEQ | 6007) | NO: | NO: 6008) | AR (SEQ | | 6062) |
| | ID NO: | | 6060) | | ID NO: | | |
| | 6059) | | | | 6061) | | |

TABLE 34
Exemplary heavy chain CDRs and FWRs of NKp30-targeting antigen binding domains
(according to the Kabat numbering scheme)

| Ab ID | VHFWR1 | VHCDR1 | VHFWR2 | VHCDR2 | VHFWR3 | VHCDR3 | VHFWR4 |
|---|---|---|---|---|---|---|---|
| 9G1-HC | QIQLQE | GYHWN | WIRQFP | YIYSSGS | RISITRD | GNWHY | WGQGT |
| | SGPGLV | (SEQ ID | GKKLE | TSYNPS | TSKNQF | FDF | MVTVSS |
| | KPSQSL | NO: | WMG | LKS | FLQLNS | (SEQ ID | (SEQ ID |
| | SLTCSV | 7313) | (SEQ ID | (SEQ ID | VTTEDT | NO: | NO: |
| | TGFSINT | | NO: | NO: | ATYYCA | 6002) | 6006) |
| | G (SEQ | | 6004) | 6001) | R (SEQ | | |
| | ID NO: | | | | ID NO: | | |
| | 7317) | | | | 6005) | | |
| 15H6-HC | QIQLQE | GYHWN | WIRQFP | YIYSSG | RISITRD | GNWHY | WGQGT |
| | SGPGLV | (SEQ ID | GKKLE | TTRYNP | TSKNQF | FDY | LVAVSS |
| | KPSQSL | NO: | WMG | SLKS | FLQLNS | (SEQ ID | (SEQ ID |
| | SLTCSV | 7313) | (SEQ ID | (SEQ ID | VTPEDT | NO: | NO: |
| | TGFSINT | | NO: | NO: | ATYYCT | 6009) | 6013) |
| | G (SEQ | | 6011) | 6008) | R (SEQ | | |
| | ID NO: | | | | ID NO: | | |
| | 7317) | | | | 6012) | | |
| 9G1-HC_1 | QIQLQE | GYHWN | WIRQPA | YIYSSGS | RVTMSR | GNWHY | WGQGT |
| | SGPGLV | (SEQ ID | GKGLE | TSYNPS | DTSKNQ | FDF | MVTVSS |
| | KPSETL | NO: | WIG | LKS | FSLKLSS | (SEQ ID | (SEQ ID |
| | SLTCTV | 7313) | (SEQ ID | (SEQ ID | VTAADT | NO: | NO: |
| | SGFSINT | | NO: | NO: | AVYYC | 6002) | 6017) |
| | G (SEQ | | 6015) | 6001) | AR (SEQ | | |
| | ID NO: | | | | ID NO: | | |
| | 7371) | | | | 6016) | | |
| 9G1-HC_2 | QIQLQE | GYHWN | WIRQHP | YIYSSGS | LVTISR | GNWHY | WGQGT |
| | SGPGLV | (SEQ ID | GKGLE | TSYNPS | DTSKNQ | FDF | MVTVSS |
| | KPSQTL | NO: | WIG | LKS | FSLKLSS | (SEQ ID | (SEQ ID |
| | SLTCTV | 7313) | (SEQ ID | (SEQ ID | VTAADT | NO: | NO: |
| | SGFSINT | | NO: | NO: | AVYYC | 6002) | 6021) |
| | G (SEQ | | 6019) | 6001) | AR (SEQ | | |
| | ID NO: | | | | ID NO: | | |
| | 7372) | | | | 6020) | | |
| 9G1-HC_3 | EIQLLES | GYHWN | WVRQA | YIYSSGS | RFTISRD | GNWHY | WGQGT |
| | GGGLV | (SEQ ID | PGKGLE | TSYNPS | TSKNTF | FDF | MVTVSS |
| | QPGGSL | NO: | WVG | LKS | YLQMN | (SEQ ID | (SEQ ID |
| | RLSCAV | 7313) | (SEQ ID | (SEQ ID | SLRAED | NO: | NO: |
| | SGFSINT | | NO: | NO: | TAVYYC | 6002) | 6025) |
| | G (SEQ | | 6023) | 6001) | AR (SEQ | | |
| | ID NO: | | | | ID NO: | | |
| | 7373) | | | | 6024) | | |
| 9G1-HC_4 | QIQLVQ | GYHWN | WVRQA | YIYSSGS | RVTITR | GNWHY | WGQGT |
| | SGAEVK | (SEQ ID | PGQGLE | TSYNPS | DTSTNT | FDF | MVTVSS |
| | KPGSSV | NO: | WMG | LKS | FYMELS | (SEQ ID | (SEQ ID |
| | KVSCKV | 7313) | (SEQ ID | (SEQ ID | SLRSED | NO: | NO: |
| | SGFSINT | | NO: | NO: | TAVYYC | 6002) | 6029) |
| | G (SEQ | | 6027) | 6001) | AR (SEQ | | |
| | ID NO: | | | | ID NO: | | |
| | 7374) | | | | 6028) | | |
| 9G1-HC_5 | EIQLVES | GYHWN | WVRQA | YIYSSGS | RFTISRD | GNWHY | WGQGT |
| | GGGLV | (SEQ ID | PGKGLE | TSYNPS | TAKNSF | FDF | MVTVSS |
| | QPGGSL | NO: | WVG | LKS | YLQMN | (SEQ ID | (SEQ ID |
| | RLSCAV | 7313) | (SEQ ID | (SEQ ID | SLRAED | NO: | NO: |
| | SGFSINT | | NO: | NO: | TAVYYC | 6002) | 6034) |
| | G (SEQ | | 6032) | 6001) | AR (SEQ | | |
| | ID NO: | | | | ID NO: | | |
| | 7375) | | | | 6033) | | |
| 9G1-HC_6 | QIQLVQ | GYHWN | WVRQA | YIYSSGS | RVTMTR | GNWHY | WGQGT |
| | SGAEVK | (SEQ ID | PGQGLE | TSYNPS | DTSTNT | FDF | MVTVSS |
| | KPGASV | NO: | WMG | LKS | FYMELS | (SEQ ID | (SEQ ID |
| | KVSCKV | 7313) | (SEQ ID | (SEQ ID | SLRSED | NO: | NO: |
| | SGFSINT | | NO: | NO: | TAVYYC | 6002) | 6038) |
| | G (SEQ | | 6036) | 6001) | AR (SEQ | | |
| | ID NO: | | | | ID NO: | | |
| | 7376) | | | | 6037) | | |
| 15H6- | QIQLQE | GYHWN | WIRQHP | YIYSSG | LVTISR | GNWHY | WGQGT |
| HC_1 | SGPGLV | (SEQ ID | GKGLE | TTRYNP | DTSKNQ | FDY | LVTVSS |
| | KPSQTL | NO: | WIG | SLKS | FSLKLSS | (SEQ ID | (SEQ ID |
| | SLTCTV | 7313) | (SEQ ID | (SEQ ID | VTAADT | NO: | NO: |
| | SGFSINT | | NO: | NO: | AVYYC | 6009) | 6042) |
| | G (SEQ | | 6040) | 6008) | AR (SEQ | | |
| | ID NO: | | | | ID NO: | | |
| | 7372) | | | | 6041) | | |
| 15H6- | QIQLQE | GYHWN | WIRQPA | YIYSSG | RVTMSR | GNWHY | WGQGT |
| HC_2 | SGPGLV | (SEQ ID | GKGLE | TTRYNP | DTSKNQ | FDY | LVTVSS |
| | KPSETL | NO: | WIG | SLKS | FSLKLSS | (SEQ ID | (SEQ ID |
| | SLTCTV | 7313) | (SEQ ID | (SEQ ID | VTAADT | NO: | NO: |
| | SGFSINT | | NO: | NO: | AVYYC | 6009) | 6046) |
| | G (SEQ | | 6044) | 6008) | AR (SEQ | | |
| | ID NO: | | | | ID NO: | | |
| | 7371) | | | | 6045) | | |
| 15H6- | EIQLLES | GYHWN | WVRQA | YIYSSG | RFTISRD | GNWHY | WGQGT |
| HC_3 | GGGLV | (SEQ ID | PGKGLE | TTRYNP | TSKNTF | FDY | LVTVSS |
| | QPGGSL | NO: | WVG | SLKS | YLQMN | (SEQ ID | (SEQ ID |
| | RLSCAV | 7313) | (SEQ ID | (SEQ ID | SLRAED | NO: | NO: |
| | SGFSINT | | NO: | NO | TAVYYC | 6009) | 6050) |
| | G (SEQ | | 6048) | 6008) | AR (SEQ | | |
| | ID NO: | | | | ID NO: | | |
| | 7373) | | | | 6049) | | |
| 15H6- | QIQLVE | GYHWN | WIRQAP | YIYSSG | RFTISRD | GNWHY | WGQGT |
| HC_4 | SGGGLV | (SEQ ID | GKGLE | TTRYNP | TAKNSF | FDY | LVTVSS |
| | KPGGSL | NO: | WVG | SLKS | YLQMN | (SEQ ID | (SEQ ID |
| | RLSCAV | 7313) | (SEQ ID | (SEQ ID | SLRAED | NO: | NO: |
| | SGFSINT | | NO: | NO: | TAVYYC | 6009) | 6054) |
| | G (SEQ | | 6052) | 6008) | AR (SEQ | | |
| | ID NO: | | | | ID NO: | | |
| | 7377) | | | | 6053) | | |
| 15H6- | QIQLVQ | GYHWN | WVRQA | YIYSSG | RVTMTR | GNWHY | WGQGT |
| HC_5 | SGAEVK | (SEQ ID | PGQGLE | TTRYNP | DTSTNT | FDY | LVTVSS |
| | KPGASV | NO: | WMG | SLKS | FYMELS | (SEQ ID | (SEQ ID |
| | KVSCKV | 7313) | (SEQ ID | (SEQ ID | SLRSED | NO: | NO: |
| | SGFSINT | | NO: | NO: | TAVYYC | 6009) | 6058) |
| | G (SEQ | | 6056) | 6008) | AR (SEQ | | |
| | ID NO: | | | | ID NO: | | |
| | 7376) | | | | 6057) | | |
| 15H6- | EIQLVQ | GYHWN | WVQQA | YIYSSG | RVTITR | GNWHY | WGQGT |
| HC_6 | SGAEVK | (SEQ ID | PGKGLE | TTRYNP | DTSTNT | FDY | LVTVSS |
| | KPGATV | NO: | WMG | SLKS | FYMELS | (SEQ ID | (SEQ ID |
| | KISCKV | 7313) | (SEQ ID | (SEQ ID | SLRSED | NO: | NO: |
| | SGFSINT | | NO: | NO: | TAVYYC | 6009) | 6062) |
| | G (SEQ | | 6060) | 6008) | AR (SEQ | | |
| | ID NO: | | | | ID NO: | | |
| | 7378) | | | | 6061) | | |
| 9D9-HC | QIQLQE | GYHWN | WIRQFP | YIYSSG | RISITRD | GDWHY | WGQGT |
| | SGPGLV | (SEQ ID | GKKVE | TTKYNP | TSKNQF | FDY | MVAVSS |
| | KPSQSL | NO: | WMG | SLKS | FLQLNS | (SEQ ID | (SEQ ID |
| | SLSCSV | 7313) | (SEQ ID | (SEQ ID | VTTEDT | NO: | NO: |
| | TGFSINT | | NO: | NO: | ATYYCA | 7315) | 7316) |
| | G (SEQ | | 7314) | 7385) | R (SEQ | | |
| | ID NO: | | | | ID NO: | | |
| | 7312) | | | | 6005) | | |
| 3A12-HC | QIQLQE | GYHWN | WIRQFP | YIYSSGS | RFSITRD | GNWHY | WGQGT |
| | SGPGLV | (SEQ ID | GKKLE | TRYNPS | TSKNQF | FDY | LVAVSS |
| | KPSQSL | NO: | WMG | LKS | FLQLNS | (SEQ ID | (SEQ ID |
| | SLTCSV | 7313) | (SEQ ID | (SEQ ID | VTTEDT | NO: | NO: |
| | TGFSINT | | NO: | NO: | ATYYCT | 6009) | 6013) |
| | G (SEQ | | 6004) | 7318) | R (SEQ | | |
| | ID NO: | | | | ID NO: | | |
| | 7317) | | | | 7319) | | |
| 12D10-HC | QIQLQE | GYHWN | WIRQFP | YIYSSG | RISITRD | GNWHY | WGQGT |
| | SGPGLV | (SEQ ID | GKKLE | TTRYNP | TSKNQF | FDY | LVAVSS |
| | KPSQSL | NO: | WMG | SLKS | FLQLNS | (SEQ ID | (SEQ ID |
| | SLTCSV | 7313) | (SEQ ID | (SEQ ID | VTPEDT | NO: | NO: |
| | TGFSINT | | NO: | NO: | ATYYCT | 6009) | 6013) |
| | G (SEQ | | 6004) | 6008) | R (SEQ | | |
| | ID NO: | | | | ID NO: | | |
| | 7317) | | | | 6012) | | |
| 15E1-HC | QIQLQE | GYHWN | WIRQFP | YIYSSGS | RFSITRD | GDWHY | WGPGT |
| | SGPGLV | (SEQ ID | GKKLE | TSYNPS | TSKNQF | FDY | MVTVSS |
| | KPSQSL | NO: | WMG | LKS | FLQLNS | (SEQ ID | (SEQ ID |
| | SLSCSV | 7313) | (SEQ ID | (SEQ ID | VTTEDT | NO: | NO: |
| | TGFSITT | | NO: | NO: | ATYYCA | 7315) | 7324) |
| | T (SEQ | | 6004) | 6001) | R (SEQ | | |
| | ID NO: | | | | ID NO: | | |
| | 7322) | | | | 7323) | | |
| 15E1 | QIQLQE | GYHWN | WIRQHP | YIYSSGS | LVTISR | GDWHY | WGQGT |
| Humanized | SGPGLV | (SEQ ID | GKGLE | TSYNPS | DTSKNQ | FDY | MVTVSS |
| variant_VH | KPSQTL | NO: | WIG | LKS | FSLKLSS | (SEQ ID | (SEQ ID |
| 1 | SLTCTV | 7313) | (SEQ ID | (SEQ ID | VTAADT | NO: | NO: |
| | SGFSITT | | NO: | NO: | AVYYC | 7315) | 6006) |
| | T (SEQ | | 6019) | 6001) | AR (SEQ | | |
| | ID NO: | | | | ID NO: | | |
| | 7330) | | | | 6020) | | |
| 15E1 | QIQLVE | GYHWN | WIRQAP | YIYSSGS | RFTISRD | GDWHY | WGQGT |
| Humanized | SGGGLV | (SEQ ID | GKGLE | TSYNPS | TAKNSF | FDY | MVTVSS |
| variant_VH | KPGGSL | NO: | WVG | LKS | YLQMN | (SEQ ID | (SEQ ID |
| 2 | RLSCAV | 7313) | (SEQ ID | (SEQ ID | SLRAED | NO: | NO: |
| | SGFSITT | | NO: | NO: | TAVYYC | 7315) | 6006) |
| | T (SEQ | | 6052) | 6001) | AR (SEQ | | |
| | ID NO: | | | | ID NO: | | |
| | 7331) | | | | 6033) | | |
| 15E1 | EIQLLES | GYHWN | WVRQA | YIYSSGS | RFTISRD | GDWHY | WGQGT |
| Humanized | GGGLV | (SEQ ID | PGKGLE | TSYNPS | TSKNTF | FDY | MVTVSS |
| variant_VH | QPGGSL | NO: | WVG | LKS | YLQMN | (SEQ ID | (SEQ ID |
| 3 | RLSCAV | 7313) | (SEQ ID | (SEQ ID | SLRAED | NO: | NO: |
| | SGFSITT | | NO: | NO: | TAVYYC | 7315) | 6006) |
| | T (SEQ | | 6023) | 6001) | AR (SEQ | | |
| | ID NO: | | | | ID NO: | | |
| | 7332) | | | | 6024) | | |
| 15E1 | EIQLVES | GYHWN | WVRQA | YIYSSGS | RFTISRD | GDWHY | WGQGT |
| Humanized | GGGLV | (SEQ ID | PGKGLE | TSYNPS | TAKNSF | FDY | MVTVSS |
| variant_VH | QPGGSL | NO: | WVG | LKS | YLQMN | (SEQ ID | (SEQ ID |
| 4 | RLSCAV | 7313) | (SEQ ID | (SEQ ID | SLRAED | NO: | NO: |
| | SGFSITT | | NO: | NO: | TAVYYC | 7315) | 6006) |
| | T (SEQ | | 6023) | 6001) | AR (SEQ | | |
| | ID NO: | | | | ID NO: | | |
| | 7333) | | | | 6033) | | |
| 15E1 | QIQLVQ | GYHWN | WVRQA | YIYSSGS | RVTMTR | GDWHY | WGQGT |
| Humanized | SGAEVK | (SEQ ID | PGQGLE | TSYNPS | DTSTNT | FDY | MVTVSS |
| variant_VH | KPGASV | NO: | WMG | LKS | FYMELS | (SEQ ID | (SEQ ID |
| 5 | KVSCKV | 7313) | (SEQ ID | (SEQ ID | SLRSED | NO: | NO: |
| | SGFSITT | | NO: | NO: | TAVYYC | 7315) | 6006) |
| | T (SEQ | | 6027) | 6001) | AR (SEQ | | |
| | ID NO: | | | | ID NO: | | |
| | 7334) | | | | 6037) | | |

TABLE 8
Exemplary light chain CDRs and FWRs of NKp30-targeting antigen binding domains

| Ab ID | VLFWR1 | VLCDR1 | VLFWR2 | VLCDR2 | VLFWR3 | VLCDR3 | VLFWR4 |
|---|---|---|---|---|---|---|---|
| 9G1-LC | SYTLTQ | SGERLS | WYQQK | ENDKRP | GIPDQFS | QSWDST | FGSGTQ |
| | PPLLSV | DKYVH | PGRAPV | S (SEQ | GSNSGN | NSAV | LTVL |
| | ALGHK | (SEQ ID | MVIY | ID NO: | IATLTIS | (SEQ ID | (SEQ ID |
| | ATITC | NO: | (SEQ ID | 6064) | KAQAG | NO: | NO: |
| | (SEQ ID | 6063) | NO: | | YEADY | 7293) | 6069) |
| | NO: | | 6067) | | YC (SEQ | | |
| | 6066) | | | | ID NO: | | |
| | | | | | 7292) | | |
| 15H6-LC | SYTLTQ | SGENLS | WYQQK | ENEKRP | GIPDQFS | HYWESI | FGSGTH |
| | PPSLSV | DKYVH | PGRAPV | S (SEQ | GSNSGN | NSVV | LTVL |
| | APGQKA | (SEQ ID | MVIY | ID NO: | IATLTIS | (SEQ ID | (SEQ ID |
| | TIIC | NO: | (SEQ ID | 6071) | KAQPGS | NO: | NO: |
| | (SEQ ID | 6070) | NO: | | EADYYC | 6072) | 6076) |
| | NO: | | 6074) | | (SEQ ID | | |
| | 6073) | | | | NO: | | |
| | | | | | 6075) | | |
| 9G1-LC_1 | QSVTTQ | SGERLS | WYQQL | ENDKRP | GVPDRF | QSWDST | FGGGTQ |
| | PPSVSG | DKYVH | PGTAPK | S (SEQ | SGSNSG | NSAV | LTVL |
| | APGQRV | (SEQ ID | MLIY | ID NO: | NSASLA | (SEQ ID | (SEQ ID |
| | TISC | NO: | (SEQ ID | 6064) | ITGLQA | NO: | NO: |
| | (SEQ ID | 6063) | NO: | | EDEADY | 7293) | 6080) |
| | NO: | | 6078) | | YC (SEQ | | |
| | 6077) | | | | ID NO: | | |
| | | | | | 6079) | | |
| 9G1-LC_2 | QSVTTQ | SGERLS | WYQQL | ENDKRP | GVPDRF | QSWDST | FGGGTQ |
| | PPSASG | DKYVH | PGTAPK | S (SEQ | SGSNSG | NSAV | LTVL |
| | TPGQRV | (SEQ ID | MLIY | ID NO: | NSASLA | (SEQ ID | (SEQ ID |
| | TISC | NO: | (SEQ ID | 6064) | ISGLQSE | NO: | NO: |
| | (SEQ ID | 6063) | NO | | DEADY | 7293) | 6084) |
| | NO: | | 6082) | | YC (SEQ | | |
| | 6081) | | | | ID NO: | | |
| | | | | | 6083) | | |
| 9G1-LC_3 | QSVTTQ | SGERLS | WYQQL | ENDKRP | GVPDRF | QSWDST | FGGGTQ |
| | PPSASG | DKYVH | PGTAPK | S (SEQ | SGSNSG | NSAV | LTVL |
| | TPGQRV | (SEQ ID | MLIY | ID NO: | NSASLA | (SEQ ID | (SEQ ID |
| | TISC | NO: | (SEQ ID | 6064) | ISGLRSE | NO | NO: |
| | (SEQ ID | 6063) | NO: | | DEADY | 7293) | 6088) |
| | NO: | | 6086) | | YC (SEQ | | |
| | 6085) | | | | ID NO: | | |
| | | | | | 6087) | | |
| 9G1-LC 4 | SSETTQ | SGERLS | WYQQK | ENDKRP | GIPERFS | QSWDST | FGGGTQ |
| | PHSVSV | DKYVH | PGQDPV | S (SEQ | GSNPGN | NSAV | LTVL |
| | ATAQM | (SEQ ID | MVIY | ID NO: | TATLTIS | (SEQ ID | (SEQ ID |
| | ARITC | NO: | (SEQ ID | 6064) | RIEAGD | NO: | NO: |
| | (SEQ ID | 6063) | NO: | | EADYYC | 7293) | 6092) |
| | NO: | | 6090) | | (SEQ ID | | |
| | 6089) | | | | NO: | | |
| | | | | | 6091) | | |
| 9G1-LC_5 | DIQMTQ | SGERLS | WYQQK | ENDKRP | GVPSRF | QSWDST | FGQGTK |
| | SPSTLSA | DKYVH | PGKAPK | S (SEQ | SGSNSG | NSAV | VEIK |
| | SVGDRV | (SEQ ID | MLIY | ID NO: | NEATLT | (SEQ ID | (SEQ ID |
| | TITC | NO: | (SEQ ID | 6064) | ISSLQPD | NO: | NO: |
| | (SEQ ID | 6063) | NO: | | DFATYY | 7293) | 6096) |
| | NO: | | 6094) | | C (SEQ | | |
| | 6093) | | | | ID NO: | | |
| | | | | | 6095) | | |
| 15H6- | QYVLTQ | SGENLS | WYQQL | ENEKRP | GVPDRF | HYWESI | FGEGTE |
| LC_1 | PPSASG | DKYVH | PGTAPK | S (SEQ | SGSNSG | NSVV | LTVL |
| | TPGQRV | (SEQ ID | MLIY | ID NO: | NSASLA | (SEQ ID | (SEQ ID |
| | TISC | NO: | (SEQ ID | 6071) | ISGLQSE | NO: | NO: |
| | (SEQ ID | 6070) | NO: | | DEADY | 6072) | 6100) |
| | NO: | | 6098) | | YC (SEQ | | |
| | 6097) | | | | ID NO: | | |
| | | | | | 6099) | | |
| 15H6- | QYVLTQ | SGENLS | WYQQL | ENEKRP | GVPDRF | HYWESI | FGEGTE |
| LC_2 | PPSASG | DKYVH | PGTAPK | S (SEQ | SGSNSG | NSVV | LTVL |
| | TPGQRV | (SEQ ID | MLIY | ID NO: | NSASLA | (SEQ ID | (SEQ ID |
| | TISC | NO: | (SEQ ID | 6071) | ISGLRSE | NO: | NO: |
| | (SEQ ID | 6070) | NO: | | DEADY | 6072) | 6104) |
| | NO: | | 6102) | | YC (SEQ | | |
| | 6101) | | | | ID NO: | | |
| | | | | | 6103) | | |
| 15H6- | SYELTQ | SGENLS | WYQQK | ENEKRP | GIPERFS | HYWESI | FGEGTE |
| LC_3 | PPSVSV | DKYVH | PGQSPV | S (SEQ | GSNSGN | NSVV | LTVL |
| | SPGQTA | (SEQ ID | MVIY | ID NO: | TATLTIS | (SEQ ID | (SEQ ID |
| | SITC | NO: | (SEQ ID | 6071) | GTQAM | NO: | NO: |
| | (SEQ ID | 6070) | NO: | | DEADY | 6072) | 6108) |
| | NO: | | 6106) | | YC (SEQ | | |
| | 6105) | | | | ID NO: | | |
| | | | | | 6107) | | |
| 15H6- | DYVLTQ | SGENLS | WYLQK | ENEKRP | GVPDRF | HYWESI | FGQGTK |
| LC_4 | SPLSLPV | DKYVH | PGQSPQ | S (SEQ | SGSNSG | NSVV | VEIK |
| | TPGEPA | (SEQ ID | MLIY | ID NO: | NDATLK | (SEQ ID | (SEQ ID |
| | SISC | NO: | (SEQ ID | 6071) | ISRVEA | NO: | NO: |
| | (SEQ ID | 6070) | NO: | | EDVGV | 6072) | 6112) |
| | NO: | | 6110) | | YYC | | |
| | 6109) | | | | (SEQ ID | | |
| | | | | | NO: | | |
| | | | | | 6111) | | |
| 15H6- | AYQLTQ | SGENLS | WYQQK | ENEKRP | GVPSRF | HYWESI | FGQGTK |
| LC_5 | SPSSLSA | DKYVH | PGKAPK | S (SEQ | SGSNSG | NSVV | VEIK |
| | SVGDRV | (SEQ ID | MLIY | ID NO: | NDATLT | (SEQ ID | (SEQ ID |
| | TITC | NO: | (SEQ ID | 6071) | ISSLQPE | NO: | NO: |
| | (SEQ ID | 6070) | NO: | | DFATYY | 6072) | 6116) |
| | NO: | | 6114) | | C (SEQ | | |
| | 6113) | | | | ID NO: | | |
| | | | | | 6115) | | |
| 15H6- | EYVLTQ | SGENLS | WYQQK | ENEKRP | GIPARFS | HYWESI | FGQGTK |
| LC_6 | SPATLS | DKYVH | PGQAPR | S (SEQ | GSNSGN | NSVV | VEIK |
| | VSPGER | (SEQ ID | MLIY | ID NO: | EATLTIS | (SEQ ID | (SEQ ID |
| | ATLSC | NO: | (SEQ ID | 6071) | SLQSED | NO: | NO: |
| | (SEQ ID | 6070) | NO: | | FAVYYC | 6072) | 6120) |
| | NO: | | 6118) | | (SEQ ID | | |
| | 6117) | | | | NO: | | |
| | | | | | 6119) | | |
| 9D9-LC | SYTLTQ | SGENLS | WYQQK | ENDKRP | GIPDQFS | HCWDS | FGSGTH |
| | PPLVSV | DKYVH | PGRAPV | S (SEQ | GSNSGN | TNSAV | LTVL |
| | ALGQK | (SEQ ID | MVIY | ID NO: | IATLTIS | (SEQ ID | (SEQ ID |
| | ATIIC | NO: | (SEQ ID | 6064) | KAQAG | NO: | NO: |
| | (SEQ ID | 6070) | NO: | | YEADY | 7321) | 6076) |
| | NO: | | 6067) | | YC (SEQ | | |
| | 7320) | | | | ID NO: | | |
| | | | | | 7292) | | |
| 3A12-LC | SYTLTQ | SGENLS | WYQQK | ENDKRP | GIPDQFS | HCWDS | FGSGTH |
| | PPLVSV | DKYVH | PGRAPV | S (SEQ | GSNSGN | TNSAV | LTVL |
| | ALGQK | (SEQ ID | MVIY | ID NO: | IATLTIS | (SEQ ID | (SEQ ID |
| | ATIIC | NO: | (SEQ ID | 6064) | KAQAG | NO: | NO: |
| | (SEQ ID | 6070) | NO: | | YEADY | 7321) | 6076) |
| | NO: | | 6067) | | YC (SEQ | | |
| | 7320) | | | | ID NO: | | |
| | | | | | 7292) | | |
| 12D10-LC | SYTLTQ | SGENLS | WYQQK | ENEKRP | GIPDQFS | HYWESI | FGSGTH |
| | PPSLSV | DKYVH | PGRAPV | S (SEQ | GSNSGN | NSVV | LTVL |
| | APGQKA | (SEQ ID | MVIY | ID NO: | IATLTIS | (SEQ ID | (SEQ ID |
| | TIIC | NO: | (SEQ ID | 6071) | KAQPGS | NO: | NO: |
| | (SEQ ID | 6070) | NO: | | EADYYC | 6072) | 6076) |
| | NO: | | 6074) | | (SEQ ID | | |
| | 6073) | | | | NO: | | |
| | | | | | 6075) | | |
| 15E1-LC | SFTLTQ | SGEKLS | WYQQK | ENDRRP | GIPDQFS | QFWDST | FGGGTQ |
| | PPLVSV | DKYVH | PGRAPV | S (SEQ | GSNSGN | NSAV | LTVL |
| | AVGQV | (SEQ ID | MVIY | ID NO: | IASLTIS | (SEQ ID | (SEQ ID |
| | ATITC | NO: | (SEQ ID | 7327) | KAQAG | NO: | NO: |
| | (SEQ ID | 7326) | NO: | | DEADYF | 7329) | 6080) |
| | NO: | | 6067) | | C (SEQ | | |
| | 7325) | | | | ID NO: | | |
| | | | | | 7328) | | |
| 15E1 | SSETTQ | SGEKLS | WYQQK | ENDRRP | GIPERFS | QFWDST | FGGGTQ |
| Humanized | PPSVSV | DKYVH | PGQSPV | S (SEQ | GSNSGN | NSAV | LTVL |
| variant_VL | SPGQTA | (SEQ ID | MVIY | ID NO: | TATLTIS | (SEQ ID | (SEQ ID |
| 1 | SITC | NO: | (SEQ ID | 7327) | GTQAM | NO: | NO: |
| | (SEQ ID | 7326) | NO: | | DEADYF | 7329) | 6080) |
| | NO: | | 6106) | | C (SEQ | | |
| | 7335) | | | | ID NO: | | |
| | | | | | 7336) | | |
| 15E1 | SSETTQ | SGEKLS | WYQQK | ENDRRP | GIPERFS | QFWDST | FGGGTQ |
| Humanized | PHSVSV | DKYVH | PGQDPV | S (SEQ | GSNPGN | NSAV | LTVL |
| variant_VL | ATAQM | (SEQ ID | MVIY | ID NO: | TATLTIS | (SEQ ID | (SEQ ID |
| 2 | ARITC | NO: | (SEQ ID | 7327) | RIEAGD | NO: | NO: |
| | (SEQ ID | 7326) | NO: | | EADYFC | 7329) | 6080) |
| | NO: | | 6090) | | (SEQ ID | | |
| | 6089) | | | | NO: | | |
| | | | | | 7337) | | |
| 15E1 | QSVTTQ | SGEKLS | WYQQL | ENDRRP | GVPDRF | QFWDST | FGGGTQ |
| Humanized | PPSASG | DKYVH | PGTAPK | S (SEQ | SGSNSG | NSAV | LTVL |
| variant_VL | TPGQRV | (SEQ ID | MLIY | ID NO: | NSASLA | (SEQ ID | (SEQ ID |
| 3 | TISC | NO: | (SEQ ID | 7327) | ISGLRSE | NO: | NO: |
| | (SEQ ID | 7326) | NO: | | DEADYF | 7329) | 6080) |
| | NO: | | 6078) | | C (SEQ | | |
| | 6081) | | | | ID NO: | | |
| | | | | | 7338) | | |
| 15E1 | QSVTTQ | SGEKLS | WYQQL | ENDRRP | GVPDRF | QFWDST | FGGGTQ |
| Humanized | PPSVSG | DKYVH | PGTAPK | S (SEQ | SGSNSG | NSAV | LTVL |
| variant_VL | APGQRV | (SEQ ID | MLIY | ID NO: | NSASLA | (SEQ ID | (SEQ ID |
| 4 | TISC | NO: | (SEQ ID | 7327) | ITGLQA | NO: | NO: |
| | (SEQ ID | 7326) | NO: | | EDEADY | 7329) | 6080) |
| | NO: | | 6078) | | FC (SEQ | | |
| | 6077) | | | | ID NO: | | |
| | | | | | 7339) | | |
| 15E1 | DSVTTQ | SGEKLS | WYQQR | ENDRRP | GVPDRF | QFWDST | FGGGTK |
| Humanized | SPLSLPV | DKYVH | PGQSPR | S (SEQ | SGSNSG | NSAV | VEIK |
| variant_VL | TLGQPA | (SEQ ID | MLIY | ID NO: | NDATLK | (SEQ ID | (SEQ ID |
| 5 | SISC | NO: | (SEQ ID | 7327) | ISRVEA | NO: | NO: 233) |
| | (SEQ ID | 7326) | NO: | | EDVGV | 7329) | |
| | NO: | | 7341) | | YFC | | |
| | 7340) | | | | (SEQ ID | | |
| | | | | | NO: | | |
| | | | | | 7342) | | |

TABLE 35
Exemplary heavy chain CDRs and FWRs of NKp30-targeting antigen binding domains

| Ab | | | | | | | |
|---|---|---|---|---|---|---|---|
| ID | VHFWR1 | VHCDR1 | VHFWR2 | VHCDR2 | VHFWR3 | VHCDR3 | VHFWR4 |
| BKM | EIQLLES | ITTTGYH | WVRQAP | YIYSSGS | RFTISRD | GDWHYF | WGQGT |
| 0138 | GGGLVQ | WN (SEQ | GKGLEW | TSYNPSL | TSKNTFY | DY (SEQ | MVTVSS |
| | PGGSLRL | ID NO: | VG (SEQ | KS (SEQ | LQMNSL | ID NO: | (SEQ ID |
| | SCAVSGF | 7498) | ID NO: | ID NO: | RAEDTA | 7315) | NO: 6006) |
| | S (SEQ ID | | 6023) | 6001) | VYYCAR | | |
| | NO: 7497) | | | | (SEQ ID | | |
| | | | | | NO: 6024) | | |
| BKM | EIQLLES | ITTTGYH | WVRQAP | YIYSSGS | RFTISRD | GDWHYF | WGQGT |
| 0139 | GGGLVQ | WN (SEQ | GKGLEW | TSYNPSL | TSKNTFY | DY (SEQ | MVTVSS |
| | PGGSLRL | ID NO: | VG (SEQ | KS (SEQ | LQMNSL | ID NO: | (SEQ ID |
| | SCAVSGF | 7498) | ID NO: | ID NO: | RAEDTA | 7315) | NO: 6006) |
| | S (SEQ ID | | 6023) | 6001) | VYYCAR | | |
| | NO: 7497) | | | | (SEQ ID | | |
| | | | | | NO: 6024) | | |
| BKM | EIQLLES | ITTIGYH | WVRQAP | YIYSSGS | RFTISRD | GDWHYE | WGQGT |
| 0140 | GGGLVQ | WN (SEQ | GKGLEW | ISYNPSL | TSKNTFY | DY (SEQ | MVTVSS |
| | PGGSLRL | ID NO: | VG (SEQ | KS (SEQ | LQMNSL | ID NO: | (SEQ ID |
| | SCAVSGF | 7498) | ID NO: | ID NO: | RAEDTA | 7315) | NO: 6006) |
| | S (SEQ ID | | 6023) | 6001) | VYYCAR | | |
| | NO: 7497) | | | | (SEQ ID | | |
| | | | | | NO: 6024) | | |
| BKM | EIQLLES | ITTIGYH | WVRQAP | YTYSSGS | RFTISRD | GDWHYF | WGQGT |
| 0141 | GGGLVQ | WN (SEQ | GKGLEW | TSYNPSL | TSKNTFY | DY (SEQ | MVTVSS |
| | PGGSLRL | ID NO: | VG (SEQ | KS (SEQ | LQMNSL | ID NO: | (SEQ ID |
| | SCAVSGF | 7498) | ID NO: | ID NO: | RAEDTA | 7315) | NO: 6006) |
| | S (SEQ ID | | 6023) | 6001) | VYYCAR | | |
| | NO: 7497) | | | | (SEQ ID | | |
| | | | | | NO: 6024) | | |
| BKM | EIQLLES | ITTTGYH | WVRQAP | YIYSSGS | RFTISRD | GDWHYF | WGQGT |
| 0142 | GGGLVQ | WN (SEQ | GKGLEW | TSYAPSL | TSKNTFY | DY (SEQ | MVTVSS |
| | PGGSLRL | ID NO: | VG (SEQ | KS (SEQ | LQMNSL | ID NO: | (SEQ ID |
| | SCAVSGF | 7498) | ID NO: | ID NO: | RAEDTA | 7315) | NO: 6006) |
| | S (SEQ ID | | 6023) | 7437) | VYYCAR | | |
| | NO: 7497) | | | | (SEQ ID | | |
| | | | | | NO: 6024) | | |
| BKM | EIQLLES | ITTIGYH | WVRQAP | YIYSSGS | RFTISRD | GDWHYF | WGQGT |
| 0143 | GGGLVQ | WN (SEQ | GKGLEW | TSYAPSL | TSKNTFY | DY (SEQ | MVTVSS |
| | PGGSLRL | ID NO: | VG (SEQ | KS (SEQ | LQMNSL | ID NO: | (SEQ ID |
| | SCAVSGF | 7498) | ID NO: | ID NO: | RAEDTA | 7315) | NO: 6006) |
| | S (SEQ ID | | 6023) | 7437) | VYYCAR | | |
| | NO: 7497) | | | | (SEQ ID | | |
| | | | | | NO: 6024) | | |
| BKM | EIQLLES | ITTTGYH | WVRQAP | YIYSSGS | RFTISRD | GDWHYF | WGQGT |
| 0144 | GGGLVQ | WN (SEQ | GKGLEW | TSYAPSL | TSKNTFY | DY (SEQ | MVTVSS |
| | PGGSLRL | ID NO: | VG (SEQ | KS (SEQ | LQMNSL | ID NO: | (SEQ ID |
| | SCAVSGF | 7498) | ID NO: | ID NO: | RAEDTA | 7315) | NO: 6006) |
| | S (SEQ ID | | 6023) | 7437) | VYYCAR | | |
| | NO: 7497) | | | | (SEQ ID | | |
| | | | | | NO: 6024) | | |
| BKM | EIQLLES | ITTTGYH | WVRQAP | YIYSSGS | RFTISRD | GDWHYF | WGQGT |
| 0145 | GGGLVQ | WN (SEQ | GKGLEW | TSYAPSL | TSKNTFY | DY (SEQ | MVTVSS |
| | PGGSLRL | ID NO: | VG (SEQ | KS (SEQ | LQMNSL | ID NO: | (SEQ ID |
| | SCAVSGF | 7498) | ID NO: | ID NO: | RAEDTA | 7315) | NO: 6006) |
| | S (SEQ ID | | 6023) | 7437) | VYYCAR | | |
| | NO: 7497) | | | | (SEQ ID | | |
| | | | | | NO: 6024) | | |

TABLE 36
Exemplary light chain CDRs and FWRs of NKp30-targeting antigen binding domains

| Ab | | | | | | | |
|---|---|---|---|---|---|---|---|
| ID | VLFWR1 | VLCDR1 | VLFWR2 | VLCDR2 | VLFWR3 | VLCDR3 | VLFWR4 |
| BKM | DSVTTQS | SGEKLSD | WYQQRP | ENDRRPS | GVPDRES | QFWDST | FGGGTK |
| 0138 | PLSLPVT | KYVH | GQSPRM | (SEQ ID | GSNSGN | ASAV | VEIK |
| | LGQPASI | (SEQ ID | LIY (SEQ | NO: 7327) | DATLKIS | (SEQ ID | (SEQ ID |
| | SC (SEQ | NO: 7326) | ID NO: | | RVEAED | NO: 7416) | NO: 233) |
| | ID NO: | | 7341) | | VGVYFC | | |
| | 7493) | | | | (SEQ ID | | |
| | | | | | NO: 7342) | | |
| BKM | DSVTTQS | SGEKLSD | WYQQRP | ENDRRPS | GVPDRFS | QFWAST | FGGGTK |
| 0139 | PLSLPVT | KYVH | GQSPRM | (SEQ ID | GSNSGN | NSAV | VEIK |
| | LGQPASI | (SEQ ID | LIY (SEQ | NO: 7496) | DATLKIS | (SEQ ID | (SEQ ID |
| | SC (SEQ | NO: 7494) | ID NO: | | RVEAED | NO: 44) | NO: 233) |
| | ID NO: | | 7495) | | VGVYFC | | |
| | 7493) | | | | (SEQ ID | | |
| | | | | | NO: 7342) | | |
| BKM | SSETTQP | SGEKLSD | WYQQKP | ENDRRPS | GIPERFS | QFWAST | FGGGTQ |
| 0140 | PSVSVSP | KYVH | GQSPVM | (SEQ ID | GSNSGN | NSAV | LTVL |
| | GQTASIT | (SEQ ID | VIY (SEQ | NO: 7327) | TATLTIS | (SEQ ID | (SEQ ID |
| | C (SEQ ID | NO: 7326) | ID NO: | | GTQAMD | NO: 44) | NO: 6080) |
| | NO: 7335) | | 6106) | | BADYFC | | |
| | | | | | (SEQ ID | | |
| | | | | | NO: 7336) | | |
| BKM | SSETTQP | SGEKLSD | WYQQKP | ENDRRPS | GIPERFS | QFWDST | FGGGTQ |
| 0141 | PSVSVSP | KYVH | GQSPVM | (SEQ ID | GSNSGN | ASAV | LTVL |
| | GQTASIT | (SEQ ID | VIY (SEQ | NO: 7327) | TATLTIS | (SEQ ID | (SEQ ID |
| | C (SEQ ID | NO: 7326) | ID NO: | | GTQAMD | NO: 7416) | NO: 6080) |
| | NO: 7335) | | 6106) | | EADYFC | | |
| | | | | | (SEQ ID | | |
| | | | | | NO: 7336) | | |
| BKM | DSVTTQS | SGEKLSD | WYQQRP | ENDRRPS | GVPDRFS | QFWDST | FGGGTK |
| 0142 | PLSLPVT | KYVH | GQSPRM | (SEQ ID | GSNSGN | NSAV | VEIK |
| | LGQPASI | (SEQ ID | LIY (SEQ | NO: 7327) | DATLKIS | (SEQ ID | (SEQ ID |
| | SC (SEQ | NO: 7326) | ID NO: | | RVEAED | NO: 7329) | NO: 233) |
| | ID NO: | | 7341) | | VGVYFC | | |
| | 7493) | | | | (SEQ ID | | |
| | | | | | NO: 7342) | | |
| BKM | SSETTQP | SGEKLSD | WYQQKP | ENDRRPS | GIPERFS | QFWDST | FGGGTQ |
| 0143 | PSVSVSP | KYVH | GQSPVM | (SEQ ID | GSNSGN | NSAV | LTVL |
| | GQTASIT | (SEQ ID | VIY (SEQ | NO: 7327) | TATLTIS | (SEQ ID | (SEQ ID |
| | C (SEQ ID | NO: 7326) | ID NO: | | GTQAMD | NO: 7329) | NO: 6080) |
| | NO: 7335) | | 6106) | | EADYFC | | |
| | | | | | (SEQ ID | | |
| | | | | | NO: 7336) | | |
| BKM | DSVTTQS | SGEKLSD | WYQQRP | ENDRRPS | GVPDRFS | QFWAST | FGGGTK |
| 0144 | PLSLPVT | KYVH | GQSPRM | (SEQ ID | GSNSGN | ASAV | VEIK |
| | LGQPASI | (SEQ ID | LIY (SEQ | NO: 7327) | DATLKIS | (SEQ ID | (SEQ ID |
| | SC (SEQ | NO: 7326) | ID NO: | | RVEAED | NO: 7447) | NO: 233) |
| | ID NO: | | 7341) | | VGVYFC | | |
| | 7493) | | | | (SEQ ID | | |
| | | | | | NO: 7342) | | |
| BKM | SSETTQP | SGEKLSD | WYQQKP | ENDRRPS | GIPERFS | QFWAST | FGGGTQ |
| 0145 | PSVSVSP | KYVH | GQSPVM | (SEQ ID | GSNSGN | ASAV | LTVL |
| | GQTASIT | (SEQ ID | VIY (SEQ | NO: 7327) | TATLTIS | (SEQ ID | (SEQ ID |
| | C (SEQ ID | NO: 7326) | ID NO: | | GTQAMD | NO: 7447) | NO: 6080) |
| | NO: 7335) | | 6106) | | EADYFC | | |
| | | | | | (SEQ ID | | |
| | | | | | NO: 7336) | | |

TABLE 9
Exemplary variable regions of NKp30-targeting antigen binding domains

| SEQ ID | | | |
|---|---|---|---|
| NO | Ab ID | Description | Sequence |
| SEQ ID | 9G1-HC | 9G1 heavy chain | QIQLQESGPGLVKPSQSLSLTCSVTGFSINTGGYH |
| NO: 6121 | | variable region | WNWIRQFPGKKLEWMGYIYSSGSTSYNPSLKSRI |
| | | | SITRDTSKNQFFLQLNSVTTEDTATYYCARGNWH |
| | | | YFDFWGQGTMVTVSS |
| SEQ ID | 15H6-HC | 15H6 heavy | QIQLQESGPGLVKPSQSLSLTCSVTGFSINTGGYH |
| NO: 6122 | | chain variable | WNWIRQFPGKKLEWMGYIYSSGTTRYNPSLKSRI |
| | | region | SITRDTSKNQFFLQLNSVTPEDTATYYCTRGNWH |
| | | | YFDYWGQGTLVAVSS |
| SEQ ID | 9G1-HC_1 | 9G1 heavy chain | QIQLQESGPGLVKPSETLSLTCTVSGFSINTGGYH |
| NO: 6123 | | variable region | WNWIRQPAGKGLEWIGYIYSSGSTSYNPSLKSRV |
| | | humanized | TMSRDTSKNQFSLKLSSVTAADTAVYYCARGNW |
| | | variant 1 | HYFDFWGQGTMVTVSS |
| SEQ ID | 9G1-HC_2 | 9G1 heavy chain | QIQLQESGPGLVKPSQTLSLTCTVSGFSINTGGYH |
| NO: 6124 | | variable region | WNWIRQHPGKGLEWIGYIYSSGSTSYNPSLKSLV |
| | | humanized | TISRDTSKNQFSLKLSSVTAADTAVYYCARGNW |
| | | variant 2 | HYFDFWGQGTMVTVSS |
| SEQ ID | 9G1-HC_3 | 9G1 heavy chain | EIQLLESGGGLVQPGGSLRLSCAVSGFSINTGGYH |
| NO: 6125 | | variable region | WNWVRQAPGKGLEWVGYIYSSGSTSYNPSLKSR |
| | | humanized | FTISRDTSKNTFYLQMNSLRAEDTAVYYCARGN |
| | | variant 3 | WHYFDFWGQGTMVTVSS |
| SEQ ID | 9G1-HC_4 | 9G1 heavy chain | QIQLVQSGAEVKKPGSSVKVSCKVSGFSINTGGY |
| NO: 6126 | | variable region | HWNWVRQAPGQGLEWMGYIYSSGSTSYNPSLKS |
| | | humanized | RVTITRDTSTNTFYMELSSLRSEDTAVYYCARGN |
| | | variant 4 | WHYFDFWGQGTMVTVSS |
| SEQ ID | 9G1-HC_5 | 9G1 heavy chain | EIQLVESGGGLVQPGGSLRLSCAVSGFSINTGGYH |
| NO: 6127 | | variable region | WNWVRQAPGKGLEWVGYIYSSGSTSYNPSLKSR |
| | | humanized | FTISRDTAKNSFYLQMNSLRAEDTAVYYCARGN |
| | | variant 5 | WHYFDFWGQGTMVTVSS |
| SEQ ID | 9G1-HC_6 | 9G1 heavy chain | QIQLVQSGAEVKKPGASVKVSCKVSGFSINTGGY |
| NO: 6128 | | variable region | HWNWVRQAPGQGLEWMGYIYSSGSTSYNPSLKS |
| | | humanized | RVTMTRDTSTNTFYMELSSLRSEDTAVYYCARG |
| | | variant 6 | NWHYFDFWGQGTMVTVSS |
| SEQ ID | 15H6- | 15H6 heavy | QIQLQESGPGLVKPSQTLSLTCTVSGFSINTGGYH |
| NO: 6129 | HC_1 | chain variable | WNWIRQHPGKGLEWIGYIYSSGTTRYNPSLKSLV |
| | | region | TISRDTSKNQFSLKLSSVTAADTAVYYCARGNW |
| | | humanized | HYFDYWGQGTLVTVSS |
| | | variant 1 | |
| SEQ ID | 15H6- | 15H6 heavy | QIQLQESGPGLVKPSETLSLTCTVSGFSINTGGYH |
| NO: 6130 | HC_2 | chain variable | WNWIRQPAGKGLEWIGYIYSSGTTRYNPSLKSRV |
| | | region | TMSRDTSKNQFSLKLSSVTAADTAVYYCARGNW |
| | | humanized | HYFDYWGQGTLVTVSS |
| | | variant 2 | |
| SEQ ID | 15H6- | 15H6 heavy | EIQLLESGGGLVQPGGSLRLSCAVSGFSINTGGYH |
| NO: 6131 | HC_3 | chain variable | WNWVRQAPGKGLEWVGYIYSSGTTRYNPSLKSR |
| | | region | FTISRDTSKNTFYLQMNSLRAEDTAVYYCARGN |
| | | humanized | WHYFDYWGQGTLVTVSS |
| | | variant 3 | |
| SEQ ID | 15H6- | 15H6 heavy | QIQLVESGGGLVKPGGSLRLSCAVSGFSINTGGY |
| NO: 6132 | HC_4 | chain variable | HWNWIRQAPGKGLEWVGYIYSSGTTRYNPSLKS |
| | | region | RFTISRDTAKNSFYLQMNSLRAEDTAVYYCARG |
| | | humanized | NWHYFDYWGQGTLVTVSS |
| | | variant 4 | |
| SEQ ID | 15H6- | 15H6 heavy | QIQLVQSGAEVKKPGASVKVSCKVSGFSINTGGY |
| NO: 6133 | HC_5 | chain variable | HWNWVRQAPGQGLEWMGYIYSSGTTRYNPSLK |
| | | region | SRVTMTRDTSTNTFYMELSSLRSEDTAVYYCARG |
| | | humanized | NWHYFDYWGQGTLVTVSS |
| | | variant 5 | |
| SEQ ID | 15H6- | 15H6 heavy | EIQLVQSGAEVKKPGATVKISCKVSGFSINTGGYH |
| NO: 6134 | HC_6 | chain variable | WNWVQQAPGKGLEWMGYIYSSGTTRYNPSLKS |
| | | region | RVTITRDTSTNTFYMELSSLRSEDTAVYYCARGN |
| | | humanized | WHYFDYWGQGTLVTVSS |
| | | variant 6 | |
| SEQ ID | 9G1-LC | 9G1 light chain | SYTLTQPPLLSVALGHKATITCSGERLSDKYVHW |
| NO: 7294 | | variable region | YQQKPGRAPVMVIYENDKRPSGIPDQFSGSNSGN |
| | | | IATLTISKAQAGYEADYYCQSWDSTNSAVFGSGT |
| | | | QLTVL |
| SEQ ID | 15H6-LC | 15H6 light chain | SYTLTQPPSLSVAPGQKATIICSGENLSDKYVHW |
| NO: 6136 | | variable region | YQQKPGRAPVMVIYENEKRPSGIPDQFSGSNSGNI |
| | | | ATLTISKAQPGSEADYYCHYWESINSVVFGSGTH |
| | | | LTVL |
| SEQ ID | 9G1-LC_1 | 9G1 light chain | QSVTTQPPSVSGAPGQRVTISCSGERLSDKYVHW |
| NO: 6137 | | variable region | YQQLPGTAPKMLIYENDKRPSGVPDRFSGSNSGN |
| | | humanized | SASLAITGLQAEDEADYYCQSWDSTNSAVFGGG |
| | | variant 1 | TQLTVL |
| SEQ ID | 9G1-LC_2 | 9G1 light chain | QSVTTQPPSASGTPGQRVTISCSGERLSDKYVHW |
| NO: 6138 | | variable region | YQQLPGTAPKMLIYENDKRPSGVPDRFSGSNSGN |
| | | humanized | SASLAISGLQSEDEADYYCQSWDSTNSAVFGGGT |
| | | variant 2 | QLTVL |
| SEQ ID | 9G1-LC_3 | 9G1 light chain | QSVTTQPPSASGTPGQRVTISCSGERLSDKYVHW |
| NO: 6139 | | variable region | YQQLPGTAPKMLIYENDKRPSGVPDRFSGSNSGN |
| | | humanized | SASLAISGLRSEDEADYYCQSWDSTNSAVFGGGT |
| | | variant 3 | QLTVL |
| SEQ ID | 9G1-LC_4 | 9G1 light chain | SSETTQPHSVSVATAQMARITCSGERLSDKYVHW |
| NO: 6140 | | variable region | YQQKPGQDPVMVIYENDKRPSGIPERFSGSNPGN |
| | | humanized | TATLTISRIEAGDEADYYCQSWDSTNSAVFGGGT |
| | | variant 4 | QLTVL |
| SEQ ID | 9G1-LC_5 | 9G1 light chain | DIQMTQSPSTLSASVGDRVTITCSGERLSDKYVH |
| NO: 6141 | | variable region | WYQQKPGKAPKMLIYENDKRPSGVPSRFSGSNS |
| | | humanized | GNEATLTISSLQPDDFATYYCQSWDSTNSAVFGQ |
| | | variant 5 | GTKVEIK |
| SEQ ID | 15H6- | 15H6 light chain | QYVLTQPPSASGTPGQRVTISCSGENLSDKYVHW |
| NO: 6142 | LC_1 | variable region | YQQLPGTAPKMLIYENEKRPSGVPDRFSGSNSGN |
| | | humanized | SASLAISGLQSEDEADYYCHYWESINSVVFGEGT |
| | | variant 1 | ELTVL |
| SEQ ID | 15H6- | 15H6 light chain | QYVLTQPPSASGTPGQRVTISCSGENLSDKYVHW |
| NO: 6143 | LC_2 | variable region | YQQLPGTAPKMLIYENEKRPSGVPDRFSGSNSGN |
| | | humanized | SASLAISGLRSEDEADYYCHYWESINSVVFGEGT |
| | | variant 2 | ELTVL |
| SEQ ID | 15H6- | 15H6 light chain | SYELTQPPSVSVSPGQTASITCSGENLSDKYVHW |
| NO: 6144 | LC_3 | variable region | YQQKPGQSPVMVIYENEKRPSGIPERFSGSNSGNT |
| | | humanized | ATLTISGTQAMDEADYYCHYWESINSVVFGEGTE |
| | | variant 3 | LTVL |
| SEQ ID | 15H6- | 15H6 light chain | DYVLTQSPLSLPVTPGEPASISCSGENLSDKYVHW |
| NO: 6145 | LC_4 | variable region | YLQKPGQSPQMLIYENEKRPSGVPDRFSGSNSGN |
| | | humanized | DATLKISRVEAEDVGVYYCHYWESINSVVFGQG |
| | | variant 4 | TKVEIK |
| SEQ ID | 15H6- | 15H6 light chain | AYQLTQSPSSLSASVGDRVTITCSGENLSDKYVH |
| NO: 6146 | LC_5 | variable region | WYQQKPGKAPKMLIYENEKRPSGVPSRFSGSNSG |
| | | humanized | NDATLTISSLQPEDFATYYCHYWESINSVVFGQG |
| | | variant 5 | TKVEIK |
| SEQ ID | 15H6- | 15H6 light chain | EYVLTQSPATLSVSPGERATLSCSGENLSDKYVH |
| NO: 6147 | LC_6 | variable region | WYQQKPGQAPRMLIYENEKRPSGIPARFSGSNSG |
| | | humanized | NEATLTISSLQSEDFAVYYCHYWESINSVVFGQG |
| | | variant 6 | TKVEIK |
| SEQ ID | 9D9-HC | 9D9 heavy chain | QIQLQESGPGLVKPSQSLSLSCSVTGFSINTGGYH |
| NO: 7295 | | variable region | WNWIRQFPGKKVEWMGYIYSSGTTKYNPSLKSRI |
| | | | SITRDTSKNQFFLQLNSVTTEDTATYYCARGDWH |
| | | | YFDYWGQGTMVAVSS |
| SEQ ID | 9D9-LC | 9D9 light chain | SYTLTQPPLVSVALGQKATIICSGENLSDKYVHW |
| NO: 7296 | | variable region | YQQKPGRAPVMVIYENDKRPSGIPDQFSGSNSGN |
| | | | IATLTISKAQAGYEADYYCHCWDSTNSAVFGSGT |
| | | | HLTVL |
| SEQ ID | 3A12-HC | 3A12 heavy | QIQLQESGPGLVKPSQSLSLTCSVTGFSINTGGYH |
| NO: 7297 | | chain variable | WNWIRQFPGKKLEWMGYIYSSGSTRYNPSLKSRF |
| | | region | SITRDTSKNQFFLQLNSVTTEDTATYYCTRGNWH |
| | | | YFDYWGQGTLVAVSS |
| SEQ ID | 3A12-LC | 3A12 light chain | SYTLTQPPLVSVALGQKATIICSGENLSDKYVHW |
| NO: 7296 | | variable region | YQQKPGRAPVMVIYENDKRPSGIPDQFSGSNSGN |
| | | | IATLTISKAQAGYEADYYCHCWDSTNSAVFGSGT |
| | | | HLTVL |
| SEQ ID | 12D10-HC | 12D10 heavy | QIQLQESGPGLVKPSQSLSLTCSVTGFSINTGGYH |
| NO: 6122 | | chain variable | WNWIRQFPGKKLEWMGYIYSSGTTRYNPSLKSRI |
| | | region | SITRDTSKNQFFLQLNSVTPEDTATYYCTRGNWH |
| | | | YFDYWGQGTLVAVSS |
| SEQ ID | 12D10-LC | 12D10 light | SYTLTQPPSLSVAPGQKATIICSGENLSDKYVHW |
| NO: 6136 | | chain variable | YQQKPGRAPVMVIYENEKRPSGIPDQFSGSNSGNI |
| | | region | ATLTISKAQPGSEADYYCHYWESINSVVFGSGTH |
| | | | LTVL |
| SEQ ID | 15E1-HC | 15E1 heavy | QIQLQESGPGLVKPSQSLSLSCSVTGFSITTTGYH |
| NO: 7298 | | chain variable | WNWIRQFPGKKLEWMGYIYSSGSTSYNPSLKSRF |
| | | region | SITRDTSKNQFFLQLNSVTTEDTATYYCARGDWH |
| | | | YFDYWGPGTMVTVSS |
| SEQ ID | 15E1-LC | 15E1 light chain | SFTLTQPPLVSVAVGQVATITCSGEKLSDKYVHW |
| NO: 7299 | | variable region | YQQKPGRAPVMVIYENDRRPSGIPDQFSGSNSGNI |
| | | | ASLTISKAQAGDEADYFCQFWDSTNSAVFGGGT |
| | | | QLTVL |
| SEQ ID | 15E1_ | 15E1 heavy | QIQLQESGPGLVKPSQTLSLTCTVSGFSITTTGYH |
| NO: 7300 | Humanized | chain variable | WNWIRQHPGKGLEWIGYIYSSGSTSYNPSLKSLV |
| | variant_VH | region | TISRDTSKNQFSLKLSSVTAADTAVYYCARGDW |
| | 1 | humanized | HYFDYWGQGTMVTVSS |
| | | variant 1 | |
| SEQ ID | 15E1_ | 15E1 heavy | QIQLVESGGGLVKPGGSLRLSCAVSGFSITTTGYH |
| NO: 7301 | Humanized | chain variable | WNWIRQAPGKGLEWVGYIYSSGSTSYNPSLKSRF |
| | variant_VH | region | TISRDTAKNSFYLQMNSLRAEDTAVYYCARGDW |
| | 2 | humanized | HYFDYWGQGTMVTVSS |
| | | variant 2 | |
| SEQ ID | 15E1_ | 15E1 heavy | EIQLLESGGGLVQPGGSLRLSCAVSGFSITTTGYH |
| NO: 7302 | Humanized | chain variable | WNWVRQAPGKGLEWVGYIYSSGSTSYNPSLKSR |
| | variant_VH | region | FTISRDTSKNTFYLQMNSLRAEDTAVYYCARGD |
| | 3 | humanized | WHYFDYWGQGTMVTVSS |
| | (BJM0407 | variant 3 | |
| | VH and | | |
| | BJM0411 | | |
| | VH) | | |
| SEQ ID | 15E1_ | 15E1 heavy | EIQLVESGGGLVQPGGSLRLSCAVSGFSITTTGYH |
| NO: 7303 | Humanized | chain variable | WNWVRQAPGKGLEWVGYIYSSGSTSYNPSLKSR |
| | variant_VH | region | FTISRDTAKNSFYLQMNSLRAEDTAVYYCARGD |
| | 4 | humanized | WHYFDYWGQGTMVTVSS |
| | | variant 4 | |
| SEQ ID | 15E1_ | 15E1 heavy | QIQLVQSGAEVKKPGASVKVSCKVSGFSITTTGY |
| NO: 7304 | Humanized | chain variable | HWNWVRQAPGQGLEWMGYIYSSGSTSYNPSLKS |
| | variant_VH | region | RVTMTRDTSTNTFYMELSSLRSEDTAVYYCARG |
| | 5 | humanized | DWHYFDYWGQGTMVTVSS |
| | | variant 5 | |
| SEQ ID | 15E1_ | 15E1 light chain | SSETTQPPSVSVSPGQTASITCSGEKLSDKYVHWY |
| NO: 7305 | Humanized | variable region | QQKPGQSPVMVIYENDRRPSGIPERFSGSNSGNTA |
| | variant_VL | humanized | TLTISGTQAMDEADYFCQFWDSTNSAVFGGGTQ |
| | 1 | variant 1 | LTVL |
| | (BJM0407 | | |
| | VL) | | |
| SEQ ID | 15E1_ | 15E1 light chain | SSETTQPHSVSVATAQMARITCSGEKLSDKYVHW |
| NO: 7306 | Humanized | variable region | YQQKPGQDPVMVIYENDRRPSGIPERFSGSNPGN |
| | variant_VL | humanized | TATLTISRIEAGDEADYFCQFWDSTNSAVFGGGT |
| | 2 | variant 2 | QLTVL |
| SEQ ID | 15E1_ | 15E1 light chain | QSVTTQPPSASGTPGQRVTISCSGEKLSDKYVHW |
| NO: 7307 | Humanized | variable region | YQQLPGTAPKMLIYENDRRPSGVPDRFSGSNSGN |
| | variant_VL | humanized | SASLAISGLRSEDEADYFCQFWDSTNSAVFGGGT |
| | 3 | variant 3 | QLTVL |
| SEQ ID | 15E1_ | 15E1 light chain | QSVTTQPPSVSGAPGQRVTISCSGEKLSDKYVHW |
| NO: 7308 | Humanized | variable region | YQQLPGTAPKMLIYENDRRPSGVPDRFSGSNSGN |
| | variant_VL | humanized | SASLAITGLQAEDEADYFCQFWDSTNSAVFGGGT |
| | 4 | variant 4 | QLTVL |
| SEQ ID | 15E1_ | 15E1 light chain | DSVTTQSPLSLPVTLGQPASISCSGEKLSDKYVHW |
| NO: 7309 | Humanized | variable region | YQQRPGQSPRMLIYENDRRPSGVPDRFSGSNSGN |
| | variant_VL | humanized | DATLKISRVEAEDVGVYFCQFWDSTNSAVFGGG |
| | 5 | variant 5 | TKVEIK |
| | (BJM0411 | | |
| | VL) | | |
| SEQ ID | BKM0138 | BKM0138 | EIQLLESGGGLVQPGGSLRLSCAVSGFSITTTGYH |
| NO: 7302 | VH | heavy chain | WNWVRQAPGKGLEWVGYIYSSGSTSYNPSLKSR |
| | | variable region | FTISRDTSKNTFYLQMNSLRAEDTAVYYCARGD |
| | | | WHYFDYWGQGTMVTVSS |
| SEQ ID | BKM0139 | BKM0139 | EIQLLESGGGLVQPGGSLRLSCAVSGFSITTTGYH |
| NO: 7302 | VH | heavy chain | WNWVRQAPGKGLEWVGYIYSSGSTSYNPSLKSR |
| | | variable region | FTISRDTSKNTFYLQMNSLRAEDTAVYYCARGD |
| | | | WHYFDYWGQGTMVTVSS |
| SEQ ID | BKM0140 | BKM0140 | EIQLLESGGGLVQPGGSLRLSCAVSGFSITTTGYH |
| NO: 7302 | VH | heavy chain | WNWVRQAPGKGLEWVGYIYSSGSTSYNPSLKSR |
| | | variable region | FTISRDTSKNTFYLQMNSLRAEDTAVYYCARGD |
| | | | WHYFDYWGQGTMVTVSS |
| SEQ ID | BKM0141 | BKM0141 | EIQLLESGGGLVQPGGSLRLSCAVSGFSITTTGYH |
| NO: 7302 | VH | heavy chain | WNWVRQAPGKGLEWVGYIYSSGSTSYNPSLKSR |
| | | variable region | FTISRDTSKNTFYLQMNSLRAEDTAVYYCARGD |
| | | | WHYFDYWGQGTMVTVSS |
| SEQ ID | BKM0142 | BKM0142 | EIQLLESGGGLVQPGGSLRLSCAVSGFSITTTGYH |
| NO: 7390 | VH | heavy chain | WNWVRQAPGKGLEWVGYIYSSGSTSYAPSLKSR |
| | | variable region | FTISRDTSKNTFYLQMNSLRAEDTAVYYCARGD |
| | | | WHYFDYWGQGTMVTVSS |
| SEQ ID | BKM0143 | BKM0143 | EIQLLESGGGLVQPGGSLRLSCAVSGFSITTTGYH |
| NO: 7390 | VH | heavy chain | WNWVRQAPGKGLEWVGYIYSSGSTSYAPSLKSR |
| | | variable region | FTISRDTSKNTFYLQMNSLRAEDTAVYYCARGD |
| | | | WHYFDYWGQGTMVTVSS |
| SEQ ID | BKM0144 | BKM0144 | EIQLLESGGGLVQPGGSLRLSCAVSGFSITTTGYH |
| NO: 7390 | VH | heavy chain | WNWVRQAPGKGLEWVGYIYSSGSTSYAPSLKSR |
| | | variable region | FTISRDTSKNTFYLQMNSLRAEDTAVYYCARGD |
| | | | WHYFDYWGQGTMVTVSS |
| SEQ ID | BKM0145 | BKM0145 | EIQLLESGGGLVQPGGSLRLSCAVSGFSITTTGYH |
| NO: 7390 | VH | heavy chain | WNWVRQAPGKGLEWVGYIYSSGSTSYAPSLKSR |
| | | variable region | FTISRDTSKNTFYLQMNSLRAEDTAVYYCARGD |
| | | | WHYFDYWGQGTMVTVSS |
| SEQ ID | BKM0138 | BKM0138 light | DSVTTQSPLSLPVTLGQPASISCSGEKLSDKYVHW |
| NO: 7395 | VL | chain variable | YQQRPGQSPRMLIYENDRRPSGVPDRFSGSNSGN |
| | | region | DATLKISRVEAEDVGVYFCQFWDSTASAVFGGG |
| | | | TKVEIK |
| SEQ ID | BKM0139 | BKM0139 light | DSVTTQSPLSLPVTLGQPASISCSGEKLSDKYVHW |
| NO: 7397 | VL | chain variable | YQQRPGQSPRMLIYENDRRPSGVPDRFSGSNSGN |
| | | region | DATLKISRVEAEDVGVYFCQFWASTNSAVFGGG |
| | | | TKVEIK |
| SEQ ID | BKM0140 | BKM0140 light | SSETTQPPSVSVSPGQTASITCSGEKLSDKYVHWY |
| NO: 7399 | VL | chain variable | QQKPGQSPVMVIYENDRRPSGIPERFSGSNSGNTA |
| | | region | TLTISGTQAMDEADYFCQFWASTNSAVFGGGTQ |
| | | | LTVL |
| SEQ ID | BKM0141 | BKM0141 light | SSETTQPPSVSVSPGQTASITCSGEKLSDKYVHWY |
| NO: 7401 | VL | chain variable | QQKPGQSPVMVIYENDRRPSGIPERFSGSNSGNTA |
| | | region | TLTISGTQAMDEADYFCQFWDSTASAVFGGGTQ |
| | | | LTVL |
| SEQ ID | BKM0142 | BKM0142 light | DSVTTQSPLSLPVTLGQPASISCSGEKLSDKYVHW |
| NO: 7309 | VL | chain variable | YQQRPGQSPRMLIYENDRRPSGVPDRFSGSNSGN |
| | | region | DATLKISRVEAEDVGVYFCQFWDSTNSAVFGGG |
| | | | TKVEIK |
| SEQ ID | BKM0143 | BKM0143 light | SSETTQPPSVSVSPGQTASITCSGEKLSDKYVHWY |
| NO: 7305 | VL | chain variable | QQKPGQSPVMVIYENDRRPSGIPERFSGSNSGNTA |
| | | region | TLTISGTQAMDEADYFCQFWDSTNSAVFGGGTQ |
| | | | LTVL |
| SEQ ID | BKM0144 | BKM0144 light | DSVTTQSPLSLPVTLGQPASISCSGEKLSDKYVHW |
| NO: 7404 | VL | chain variable | YQQRPGQSPRMLIYENDRRPSGVPDRFSGSNSGN |
| | | region | DATLKISRVEAEDVGVYFCQFWASTASAVFGGG |
| | | | TKVEIK |
| SEQ ID | BKM0145 | BKM0145 light | SSETTQPPSVSVSPGQTASITCSGEKLSDKYVHWY |
| NO: 7405 | VL | chain variable | QQKPGQSPVMVIYENDRRPSGIPERFSGSNSGNTA |
| | | region | TLTISGTQAMDEADYFCQFWASTASAVFGGGTQ |
| | | | LTVL |

TABLE 10
Exemplary NKp30-targeting antigen binding domains/antibody molecules

| SEQ ID | | | |
|---|---|---|---|
| NO | Ab ID | Description | Sequence |
| SEQ ID | Ch(anti- | 9G1 heavy | QIQLQESGPGLVKPSQSLSLTCSVTGFSINTGGYHWN |
| NO: | NKp30 | chain | WIRQFPGKKLEWMGYIYSSGSTSYNPSLKSRISITRD |
| 6148 | 9G1)HC | | TSKNQFFLQLNSVTTEDTATYYCARGNWHYFDFWG |
| | N297A | | QGTMVTVSSASTKGPSVFPLAPSSKSTSGGTAALGC |
| | | | LVKDYFPEPVTVSWNSGALTSGVHTFPAVLQSSGLY |
| | | | SLSSVVTVPSSSLGTQTYICNVNHKPSNTKVDKRVE |
| | | | PKSCDKTHTCPPCPAPELLGGPSVFLFPPKPKDTLMI |
| | | | SRTPEVTCVVVDVSHEDPEVKFNWYVDGVEVHNA |
| | | | KTKPREEQYASTYRVVSVLTVLHQDWLNGKEYKC |
| | | | KVSNKALPAPIEKTISKAKGQPREPQVCTLPPSREEM |
| | | | TKNQVSLSCAVKGFYPSDIAVEWESNGQPENNYKT |
| | | | TPPVLDSDGSFFLVSKLTVDKSRWQQGNVFSCSVM |
| | | | HEALHNHYTQKSLSLSPGK |
| SEQ ID | Ch(anti- | 9G1 heavy | QIQLQESGPGLVKPSQSLSLTCSVTGFSINTGGYHWN |
| NO: | NKp30 | chain | WIRQFPGKKLEWMGYIYSSGSTSYNPSLKSRISITRD |
| 6149 | 9G1)HC | | TSKNQFFLQLNSVTTEDTATYYCARGNWHYFDFWG |
| | | | QGTMVTVSSASTKGPSVFPLAPSSKSTSGGTAALGC |
| | | | LVKDYFPEPVTVSWNSGALTSGVHTFPAVLQSSGLY |
| | | | SLSSVVTVPSSSLGTQTYICNVNHKPSNTKVDKRVE |
| | | | PKSCDKTHTCPPCPAPELLGGPSVFLFPPKPKDTLMI |
| | | | SRTPEVTCVVVDVSHEDPEVKFNWYVDGVEVHNA |
| | | | KTKPREEQYNSTYRVVSVLTVLHQDWLNGKEYKC |
| | | | KVSNKALPAPIEKTISKAKGQPREPQVCTLPPSREEM |
| | | | TKNQVSLSCAVKGFYPSDIAVEWESNGQPENNYKT |
| | | | TPPVLDSDGSFFLVSKLTVDKSRWQQGNVFSCSVM |
| | | | HEALHNHYTQKSLSLSPGK |
| SEQ ID | Ch(anti- | 9G1 light | SYTLTQPPLLSVALGHKATITCSGERLSDKYVHWYQ |
| NO: | NKp30 | chain | QKPGRAPVMVIYENDKRPSGIPDQFSGSNSGNIATLT |
| 6150 | 9G1)LC | | ISKAQAGYEADYYCQSWDSTNSAVFGSGTQLTVLG |
| | | | QPKANPTVTLFPPSSEELQANKATLVCLISDFYPGAV |
| | | | TVAWKADGSPVKAGVETTKPSKQSNNKYAASSYLS |
| | | | LTPEQWKSHRSYSCQVTHEGSTVEKTVAPTECS |
| SEQ ID | Ch(anti- | 15H6 | QIQLQESGPGLVKPSQSLSLTCSVTGFSINTGGYHWN |
| NO: | NKp30 | heavy | WIRQFPGKKLEWMGYIYSSGTTRYNPSLKSRISITRD |
| 6151 | 15H6)HC | chain | TSKNQFFLQLNSVTPEDTATYYCTRGNWHYFDYWG |
| | N297A | | QGTLVAVSSASTKGPSVFPLAPSSKSTSGGTAALGC |
| | | | LVKDYFPEPVTVSWNSGALTSGVHTFPAVLQSSGLY |
| | | | SLSSVVTVPSSSLGTQTYICNVNHKPSNTKVDKRVE |
| | | | PKSCDKTHTCPPCPAPELLGGPSVFLFPPKPKDTLMI |
| | | | SRTPEVTCVVVDVSHEDPEVKFNWYVDGVEVHNA |
| | | | KTKPREEQYASTYRVVSVLTVLHQDWLNGKEYKC |
| | | | KVSNKALPAPIEKTISKAKGQPREPQVCTLPPSREEM |
| | | | TKNQVSLSCAVKGFYPSDIA VEWESNGQPENNYKT |
| | | | TPPVLDSDGSFFLVSKLTVDKSRWQQGNVFSCSVM |
| | | | HEALHNHYTQKSLSLSPGK |
| SEQ ID | Ch(anti- | 15H6 | QIQLQESGPGLVKPSQSLSLTCSVTGFSINTGGYHWN |
| NO: | NKp30 | heavy | WIRQFPGKKLEWMGYIYSSGTTRYNPSLKSRISITRD |
| 6152 | 15H6)HC | chain | TSKNQFFLQLNSVTPEDTATYYCTRGNWHYFDYWG |
| | (hole) | | QGTLVAVSSASTKGPSVFPLAPSSKSTSGGTAALGC |
| | | | LVKDYFPEPVTVSWNSGALTSGVHTFPAVLQSSGLY |
| | | | SLSSVVTVPSSSLGTQTYICNVNHKPSNTKVDKRVE |
| | | | PKSCDKTHTCPPCPAPELLGGPSVFLFPPKPKDTLMI |
| | | | SRTPEVTCVVVDVSHEDPEVKFNWYVDGVEVHNA |
| | | | KTKPREEQYNSTYRVVSVLTVLHQDWLNGKEYKC |
| | | | KVSNKALPAPIEKTISKAKGQPREPQVCTLPPSREEM |
| | | | TKNQVSLSCAVKGFYPSDIAVEWESNGQPENNYKT |
| | | | TPPVLDSDGSFFLVSKLTVDKSRWQQGNVFSCSVM |
| | | | HEALHNHYTQKSLSLSPGK |
| SEQ ID | Ch(anti- | 15H6 light | SYTLTQPPSLSVAPGQKATIICSGENLSDKYVHWYQ |
| NO: | NKp30 | chain | QKPGRAPVMVIYENEKRPSGIPDQFSGSNSGNIATLT |
| 6153 | 15H6)LC | | ISKAQPGSEADYYCHYWESINSVVFGSGTHLTVLGQ |
| | | | PKANPTVTLFPPSSEELQANKATLVCLISDFYPGAVT |
| | | | VAWKADGSPVKAGVETTKPSKQSNNKYAASSYLSL |
| | | | TPEQWKSHRSYSCQVTHEGSTVEKTVAPTECS |
| SEQ ID | anti-NKp30 | Hamster | QIQLQESGPGLVKPSQSLSLTCSVTGFSINTGGYHWN |
| NO: | 9G1 scFv | anti- | WIRQFPGKKLEWMGYIYSSGSTSYNPSLKSRISITRD |
| 6187 | (VH-VL) | NKp30 | TSKNQFFLQLNSVTTEDTATYYCARGNWHYFDFWG |
| | | scFv of | QGTMVTVSSGGGGSGGGGGGGGSGGGGSSYTLTQ |
| | | 9G1 in VH | PPLLSVALGHKATITCSGERLSDKYVHWYQQKPGR |
| | | to VL | APVMVIYENDKRPSGIPDQFSGSNSGNIATLTISKAQ |
| | | orientation | AGYEADYYCQSWDSTNSAVFGSGTQLTVL |
| SEQ ID | anti-NKp30 | Hamster | SYTLTQPPLLSVALGHKATITCSGERLSDKYVHWYQ |
| NO: | 9G1 scFv | anti- | QKPGRAPVMVIYENDKRPSGIPDQFSGSNSGNIATLT |
| 6188 | (VL-VH) | NKp30 | ISKAQAGYEADYYCQSWDSTNSAVFGSGTQLTVLG |
| | | scFv of | GGGSGGGGSGGGGSGGGGSQIQLQESGPGLVKPSQ |
| | | 9G1 in VL | SLSLTCSVTGFSINTGGYHWNWIRQFPGKKLEWMG |
| | | to VH | YIYSSGSTSYNPSLKSRISITRDTSKNQFFLQLNSVTT |
| | | orientation | EDTATYYCARGNWHYFDFWGQGTMVTVSS |
| SEQ ID | anti-NKp30 | Hamster | QIQLQESGPGLVKPSQSLSLTCSVTGFSINTGGYHWN |
| NO: | 15H6 scFv | anti- | WIRQFPGKKLEWMGYIYSSGTTRYNPSLKSRISITRD |
| 6189 | (VH-VL) | NKp30 | TSKNQFFLQLNSVTPEDTATYYCTRGNWHYFDYWG |
| | | scFv of | QGTLVAVSSGGGGSGGGGSGGGGSGGGGSSYTLTQ |
| | | 15H6 in | PPSLSVAPGQKATIICSGENLSDKYVHWYQQKPGRA |
| | | VH to VL | PVMVIYENEKRPSGIPDQFSGSNSGNIATLTISKAQP |
| | | orientation | GSEADYYCHYWESINSVVFGSGTHLTVL |
| SEQ ID | anti-NKp30 | Hamster | SYTLTQPPSLSVAPGQKATIICSGENLSDKYVHWYQ |
| NO: | 15H6 scFv | anti- | QKPGRAPVMVIYENEKRPSGIPDQFSGSNSGNIATLT |
| 6190 | (VL-VH) | NKp30 | ISKAQPGSEADYYCHYWESINSVVFGSGTHLTVLGG |
| | | scFv of | GGSGGGGSGGGGSGGGGSQIQLQESGPGLVKPSQSL |
| | | 15H6 in | SLTCSVTGFSINTGGYHWNWIRQFPGKKLEWMGYI |
| | | VL to VH | YSSGTTRYNPSLKSRISITRDTSKNQFFLQLNSVTPED |
| | | orientation | TATYYCTRGNWHYFDYWGQGTLVAVSS |
| SEQ ID | BJM0859 | | EIQLLESGGGLVQPGGSLRLSCAVSGFSITTTGYHW |
| NO: | lambda scFv | | NWVRQAPGKGLEWVGYIYSSGSTSYNPSLKSRFTIS |
| 7310 | | | RDTSKNTFYLQMNSLRAEDTAVYYCARGDWHYFD |
| | | | YWGQGTMVTVSSGGGGSGGGGSGGGGSGGGGSSS |
| | | | ETTQPPSVSVSPGQTASITCSGEKLSDKYVHWYQQK |
| | | | PGQSPVMVIYENDRRPSGIPERFSGSNSGNTATLTIS |
| | | | GTQAMDEADYFCQFWDSTNSAVFGGGTQLTVL |
| SEQ ID | BJM0860 | | EIQLLESGGGLVQPGGSLRLSCAVSGFSITTTGYHW |
| NO: | kappa scFv | | NWVRQAPGKGLEWVGYIYSSGSTSYNPSLKSRFTIS |
| 7311 | | | RDTSKNTFYLQMNSLRAEDTAVYYCARGDWHYFD |
| | | | YWGQGTMVTVSSGGGGSGGGGSGGGGSGGGGSDS |
| | | | VTTQSPLSLPVTLGQPASISCSGEKLSDKYVHWYQQ |
| | | | RPGQSPRMLIYENDRRPSGVPDRFSGSNSGNDATLKI |
| | | | SRVEAEDVGVYFCQFWDSTNSAVFGGGTKVEIK |
| SEQ ID | BKM0138 | | EIQLLESGGGLVQPGGSLRLSCAVSGFSITTTGYHW |
| NO: | scFv | | NWVRQAPGKGLEWVGYIYSSGSTSYNPSLKSRFTIS |
| 7406 | | | RDTSKNTFYLQMNSLRAEDTAVYYCARGDWHYFD |
| | | | YWGQGTMVTVSSGGGGSGGGGSGGGGSGGGGSDS |
| | | | VTTQSPLSLPVTLGQPASISCSGEKLSDKYVHWYQQ |
| | | | RPGQSPRMLIYENDRRPSGVPDRFSGSNSGNDATLKI |
| | | | SRVEAEDVGVYFCQFWDSTASAVFGGGTKVEIK |
| SEQ ID | BKM0139 | | EIQLLESGGGLVQPGGSLRLSCAVSGFSITTTGYHW |
| NO: | scFv | | NWVRQAPGKGLEWVGYIYSSGSTSYNPSLKSRFTIS |
| 7407 | | | RDTSKNTFYLQMNSLRAEDTAVYYCARGDWHYFD |
| | | | YWGQGTMVTVSSGGGGSGGGGSGGGGSGGGGSDS |
| | | | VTTQSPLSLPVTLGQPASISCSGEKLSDKYVHWYQQ |
| | | | RPGQSPRMLIYENDRRPSGVPDRFSGSNSGNDATLKI |
| | | | SRVEAEDVGVYFCQFWASTNSAVFGGGTKVEIK |
| SEQ ID | BKM0140 | | EIQLLESGGGLVQPGGSLRLSCAVSGFSITTTGYHW |
| NO: | scFv | | NWVRQAPGKGLEWVGYIYSSGSTSYNPSLKSRFTIS |
| 7408 | | | RDTSKNTFYLQMNSLRAEDTAVYYCARGDWHYFD |
| | | | YWGQGTMVTVSSGGGGSGGGGSGGGGSGGGGSSS |
| | | | ETTQPPSVSVSPGQTASITCSGEKLSDKYVHWYQQK |
| | | | PGQSPVMVIYENDRRPSGIPERFSGSNSGNTATLTIS |
| | | | GTQAMDEADYFCQFWASTNSAVFGGGTQLTVL |
| SEQ ID | BKM0141 | | EIQLLESGGGLVQPGGSLRLSCAVSGFSITTTGYHW |
| NO: | scFv | | NWVRQAPGKGLEWVGYIYSSGSTSYNPSLKSRFTIS |
| 7409 | | | RDTSKNTFYLQMNSLRAEDTAVYYCARGDWHYFD |
| | | | YWGQGTMVTVSSGGGGSGGGGSGGGGSGGGGSSS |
| | | | ETTQPPSVSVSPGQTASITCSGEKLSDKYVHWYQQK |
| | | | PGQSPVMVIYENDRRPSGIPERFSGSNSGNTATLTIS |
| | | | GTQAMDEADYFCQFWDSTASAVFGGGTQLTVL |
| SEQ ID | BKM0142 | | EIQLLESGGGLVQPGGSLRLSCAVSGFSITTTGYHW |
| NO: | scFv | | NWVRQAPGKGLEWVGYIYSSGSTSYAPSLKSRFTIS |
| 7411 | | | RDTSKNTFYLQMNSLRAEDTAVYYCARGDWHYFD |
| | | | YWGQGTMVTVSSGGGGSGGGGSGGGGSGGGGSDS |
| | | | VTTQSPLSLPVTLGQPASISCSGEKLSDKYVHWYQQ |
| | | | RPGQSPRMLIYENDRRPSGVPDRFSGSNSGNDATLKI |
| | | | SRVEAEDVGVYFCQFWDSTNSAVFGGGTKVEIK |
| SEQ ID | BKM0143 | | EIQLLESGGGLVQPGGSLRLSCAVSGFSITTTGYHW |
| NO: | scFv | | NWVRQAPGKGLEWVGYIYSSGSTSYAPSLKSRFTIS |
| 7412 | | | RDTSKNTFYLQMNSLRAEDTAVYYCARGDWHYFD |
| | | | YWGQGTMVTVSSGGGGSGGGGSGGGGSGGGGSSS |
| | | | ETTQPPSVSVSPGQTASITCSGEKLSDKYVHWYQQK |
| | | | PGQSPVMVIYENDRRPSGIPERFSGSNSGNTATLTIS |
| | | | GTQAMDEADYFCQFWDSTNSAVFGGGTQLTVL |
| SEQ ID | BKM0144 | | EIQLLESGGGLVQPGGSLRLSCAVSGFSITTTGYHW |
| NO: | scFv | | NWVRQAPGKGLEWVGYIYSSGSTSYAPSLKSRFTIS |
| 7413 | | | RDTSKNTFYLQMNSLRAEDTAVYYCARGDWHYFD |
| | | | YWGQGTMVTVSSGGGGSGGGGSGGGGSGGGGSDS |
| | | | VTTQSPLSLPVTLGQPASISCSGEKLSDKYVHWYQQ |
| | | | RPGQSPRMLIYENDRRPSGVPDRFSGSNSGNDATLKI |
| | | | SRVEAEDVGVYFCQFWASTASAVFGGGTKVEIK |
| SEQ ID | BKM0145 | | EIQLLESGGGLVQPGGSLRLSCAVSGFSITTTGYHW |
| NO: | scFv | | NWVRQAPGKGLEWVGYIYSSGSTSYAPSLKSRFTIS |
| 7414 | | | RDTSKNTFYLQMNSLRAEDTAVYYCARGDWHYFD |
| | | | YWGQGTMVTVSSGGGGSGGGGSGGGGSGGGGSSS |
| | | | ETTQPPSVSVSPGQTASITCSGEKLSDKYVHWYQQK |
| | | | PGQSPVMVIYENDRRPSGIPERFSGSNSGNTATLTIS |
| | | | GTQAMDEADYFCQFWASTASAVFGGGTQLTVL |

In some embodiments, the NK cell engager is an antigen binding domain that binds to NKp46 (e.g., NKp46 present, e.g., expressed or displayed, on the surface of an NK cell) and comprises any CDR amino acid sequence, framework region (FWR) amino acid sequence, or variable region amino acid sequence disclosed in Table 15. In some embodiments, binding of the NK cell engager, e.g., antigen binding domain that binds to NKp46, to the NK cell activates the NK cell. An antigen binding domain that binds to NKp46 (e.g., NKp46 present, e.g., expressed or displayed, on the surface of an NK cell) may be said to target NKp46, the NK cell, or both.

In some embodiments, the NK cell engager is an antigen binding domain that binds to NKG2D (e.g., NKG2D present, e.g., expressed or displayed, on the surface of an NK cell) and comprises any CDR amino acid sequence, framework region (FWR) amino acid sequence, or variable region amino acid sequence disclosed in Table 15. In some embodiments, binding of the NK cell engager, e.g., antigen binding domain that binds to NKG2D, to the NK cell activates the NK cell. An antigen binding domain that binds to NKG2D (e.g., NKG2D present, e.g., expressed or displayed, on the surface of an NK cell) may be said to target NKG2D, the NK cell, or both. 006851 In some embodiments, the NK cell engager is an antigen binding domain that binds to CD16 (e.g., CD16 present, e.g., expressed or displayed, on the surface of an NK cell) and comprises any CDR amino acid sequence, framework region (FWR) amino acid sequence, or variable region amino acid sequence disclosed in Table 15. In some embodiments, binding of the NK cell engager, e.g., antigen binding domain that binds to CD16, to the NK cell activates the NK cell. An antigen binding domain that binds to CD16 (e.g., CD16 present, e.g., expressed or displayed, on the surface of an NK cell) may be said to target CD16, the NK cell, or both.

TABLE 15
Exemplary variable regions of NKp46, NKG2D, or CD16-targeting antigen binding
domains

| SEQ | | | |
|---|---|---|---|
| ID NO | Ab ID | Description | Sequence |
| SEQ | NKG2D_ | scFv that binds | QVHLQESGPGLVKPSETLSLTCTVSDDSISSYYWSWIR |
| ID NO: | 1scFv | NKG2D | QPPGKGLEWIGHISYSGSANYNPSLKSRVTISVDTSKNQ |
| 6175 | | | FSLKLSSVTAADTAVYYCANWDDAFNIWGQGTMVTV |
| | | | SSGGGGSGGGGSGGGGSGGGGSEIVLTQSPGTLSLSPG |
| | | | ERATLSCRASQSVSSSYLAWYQQKPGQAPRLLIYGASS |
| | | | RATGIPDRFSGSGSGTDFTLTISRLEPEDFAVYYCQQYG |
| | | | SSPWTFGQGTKVEIK |
| SEQ | NKG2D_ | VH that binds | QVHLQESGPGLVKPSETLSLTCTVSDDSISSYYWSWIR |
| ID NO: | 1VH | NKG2D | QPPGKGLEWIGHISYSGSANYNPSLKSRVTISVDTSKNQ |
| 6176 | | | FSLKLSSVTAADTAVYYCANWDDAFNIWGQGTMVTV |
| | | | SS |
| SEQ | NKG2D_ | VL that binds | EIVLTQSPGTLSLSPGERATLSCRASQSVSSSYLAWYQQ |
| ID NO: | 1VL | NKG2D | KPGQAPRLLIYGASSRATGIPDRFSGSGSGTDFTLTISRL |
| 6177 | | | EPEDFAVYYCQQYGSSPWTFGQGTKVEIK |
| SEQ | NKG2D_ | scFv that binds | EVQLVQSGAEVKEPGESLKISCKNSGYSFTNYWVGWV |
| ID NO: | 2scFv | NKG2D | RQMPGKGLEWMGIIYPGDSDTRYSPSFQGQVTISADKS |
| 6178 | | | INTAYLQWSSLKASDTAMYYCGRLTMFRGIIIGYFDY |
| | | | WGQGTLVTVSSGGGGSGGGGSGGGGSGGGGSEIVLTQ |
| | | | SPATLSLSPGERATLSCRASQSVSSYLAWYQQKPGQAP |
| | | | RLLIYDASNRATGIPARFSGSGSGTDFTLTISSLEPEDFA |
| | | | VYYCQQRSNWPWTFGQGTKVEIK |
| SEQ | NKG2D_ | VH that binds | EVQLVQSGAEVKEPGESLKISCKNSGYSFTNYWVGWV |
| ID NO: | 2VH | NKG2D | RQMPGKGLEWMGIIYPGDSDTRYSPSFQGQVTISADKS |
| 6179 | | | INTAYLQWSSLKASDTAMYYCGRLTMFRGIIIGYFDY |
| | | | WGQGTLVTVSS |
| SEQ | NKG2D_ | VL that binds | EIVLTQSPATLSLSPGERATLSCRASQSVSSYLAWYQQ |
| ID NO: | 2VL | NKG2D | KPGQAPRLLIYDASNRATGIPARFSGSGSGTDFTLTISSL |
| 6180 | | | EPEDFAVYYCQQRSNWPWTFGQGTKVEIK |
| SEQ | NKp46 | scFv that binds | QVQLQQSGPELVKPGASVKMSCKASGYTFTDYVINWG |
| ID NO: | scFv | NKp46 | KQRSGQGLEWIGEIYPGSGTNYYNEKFKAKATLTADK |
| 6181 | | | SSNIAYMQLSSLTSEDSAVYFCARRGRYGLYAMDYWG |
| | | | QGTSVTVSSGGGGGGGGSGGGGSGGGGSDIQMTQTT |
| | | | SSLSASLGDRVTISCRASQDISNYLNWYQQKPDGTVKL |
| | | | LIYYTSRLHSGVPSRFSGSGSGTDYSLTINNLEQEDIAT |
| | | | YFCQQGNTRPWTFGGGTKLEIK |
| SEQ | NKp46 | VH that binds | QVQLQQSGPELVKPGASVKMSCKASGYTFTDYVINWG |
| ID NO: | VH | NKp46 | KQRSGQGLEWIGEIYPGSGTNYYNEKFKAKATLTADK |
| 6182 | | | SSNIAYMQLSSLTSEDSAVYFCARRGRYGLYAMDYWG |
| | | | QGTSVTVSS |
| SEQ | NKp46 | VL that binds | DIQMTQTTSSLSASLGDRVTISCRASQDISNYLNWYQQ |
| ID NO: | VL | NKp46 | KPDGTVKLLIYYTSRLHSGVPSRFSGSGSGTDYSLTINN |
| 6183 | | | LEQEDIATYFCQQGNTRPWTFGGGTKLEIK |
| SEQ | CD16 | scFv that binds | EVQLVESGG GVVRPGGSLR LSCAASGFTF |
| ID NO: | scFv | CD16 | DDYGMSWVRQ APGKGLEWVS |
| 6184 | | | GINWNGGSTG YADSVKGRFT ISRDNAKNSL |
| | | | YLQMNSLRAE DTAVYYCARG |
| | | | RSLLFDYWGQ GTLVTVSRGG GGSGGGGSGG |
| | | | GGSSELTQDP AVSVALGQTV |
| | | | RITCQGDSLR SYYASWYQQK PGQAPVLVIY |
| | | | GKNNRPSGIP DRFSGSSSGN |
| | | | TASLTITGAQ AEDEADYYCN SRDSSGNHVV |
| | | | FGGGTKLTVL |
| SEQ | CD16V | VH that binds | EVQLVESGG GVVRPGGSLR LSCAASGFTF |
| ID NO: | H | CD16 | DDYGMSWVRQ APGKGLEWVS |
| 6185 | | | GINWNGGSTG YADSVKGRFT ISRDNAKNSL |
| | | | YLQMNSLRAE DTAVYYCARG |
| | | | RSLLFDYWGQ GTLVTVSR |
| SEQ | CD16V | VL that binds | SSELTQDP AVSVALGQTVRITCQGDSLR |
| ID NO: | L | CD16 | SYYASWYQQK PGQAPVLVIY GKNNRPSGIP |
| 6186 | | | DRFSGSSSGNTASLTITGAQ AEDEADYYCN |
| | | | SRDSSGNHVV FGGGTKLTVL |

In one embodiment, the NK cell engager is a ligand of NKp30, e.g., is a B37-6, e.g., comprises the amino acid sequence of:

(SEQ ID NO: 6198)
DLKVEMMAGGTQITPLNDNVTIFCNIFYSQPLNITSMGITWFWKSLTFD
KEVKVFEFFGDHQEAFRPGAIVSPWRLKSGDASLRLPGIQLEEAGEYRC
EVVVTPLKAQGTVQLEVVASPASRLLLDQVGMKENEDKYMCESSGFYPE
AINITWEKQTQKFPHPIEISEDVITGPTIKNMDGTFNVTSCLKLNSSQE
DPGTVYQCVVRHASLHTPLRSNFTLTAARHSLSETEKTDNFS,

a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 6198.

In other embodiments, the NK cell engager is a ligand of NKp44 or NKp46, which is a viral HA. Viral hemagglutinins (HA) are glyco proteins which are on the surface of viruses. HA proteins allow viruses to bind to the membrane of cells via sialic acid sugar moieties which contributes to the fusion of viral membranes with the cell membranes (see e.g., Eur J Immunol. 2001 Sep.; 31(9):2680-9 “Recognition of viral hemagglutinins by NKp44 but not by NKp30”; and Nature. 2001 Feb. 22; 409(6823):1055-60 “Recognition of haemagglutinins on virus-infected cells by NKp46 activates lysis by human NK cells” the contents of each of which are incorporated by reference herein).

In other embodiments, the NK cell engager is a ligand of NKG2D chosen from MICA, MICB, or ULBP1, e.g., wherein:

(i) MICA comprises the amino acid sequence:

(SEQ ID NO: 6199)
EPHSLRYNLTVLSWDGSVQSGFLTEVHLDGQPFLRCDRQKCRAKPQGQW
AEDVLGNKTWDRETRDLTGNGKDLRMTLAHIKDQKEGLHSLQEIRVCEI
HEDNSTRSSQHFYYDGELFLSQNLETKEWTMPQSSRAQTLAMNVRNFLK
EDAMKTKTHYHAMHADCLQELRRYLKSGVVLRRTVPPMVNVTRSEASEG
NITVTCRASGFYPWNITLSWRQDGVSLSHDTQQWGDVLPDGNGTYQTWV
ATRICQGEEQRFTCYMEHSGNHSTHPVPSGKVLVLQSHW,

a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 6199;
(ii) MICB comprises the amino acid sequence:

(SEQ ID NO: 6200)
AEPHSLRYNLMVLSQDESVQSGFLAEGHLDGQPFLRYDRQKRRAKPQGQ
WAEDVLGAKTWDTETEDLTENGQDLRRTLTHIKDQKGGLHSLQEIRVCE
IHEDSSTRGSRHFYYDGELFLSQNLETQESTVPQSSRAQTLAMNVTNFW
KEDAMKTKTHYRAMQADCLQKLQRYLKSGVAIRRTVPPMVNVTCSEVSE
GNITVTCRASSFYPRNITLTWRQDGVSLSHNTQQWGDVLPDGNGTYQTW
VATRIRQGEEQRFTCYMEHSGNHGTHPVPSGKVLVLQSQRTD,

a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 6200; or
(iii) ULBP1 comprises the amino acid sequence:

(SEQ ID NO: 6201)
GWVDTHCLCYDFIITPKSRPEPQWCEVQGLVDERPFLHYDCVNHKAKAF
ASLGKKVNVTKTWEEQTETLRDVVDFLKGQLLDIQVENLIPIEPLTLQA
RMSCEHEAHGHGRGSWQFLFNGQKFLLFDSNNRKWTALHPGAKKMTEKW
EKNRDVTMFFQKISLGDCKMWLEEFLMYWEQMLDPTKPPSLAPG,

a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 6201.

In other embodiments, the NK cell engager is a ligand of DNAM1 chosen from NECTIN2 or NECL5, e.g., wherein:
(i) NECTIN2 comprises the amino acid sequence:

(SEQ ID NO: 6202)
QDVRVQVLPEVRGQLGGTVELPCHLLPPVPGLYISLVTWQRPDAPANHQ
NVAAFHPKMGPSFPSPKPGSERLSFVSAKQSTGQDTEAELQDATLALHG
LTVEDEGNYTCEFATFPKGSVRGMTWLRVIAKPKNQAEAQKVTFSQDPT
TVALCISKEGRPPARISWLSSLDWEAKETQVSGTLAGTVTVTSRFTLVP
SGRADGVTVTCKVEHESFEEPALIPVTLSVRYPPEVSISGYDDNWYLGR
TDATLSCDVRSNPEPTGYDWSTTSGTFPTSAVAQGSQLVIHAVDSLFNT
TFVCTVTNAVGMGRAEQVIFVRETPNTAGAGATGG,

a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 6202; or
(ii) NECL5 comprises the amino acid sequence:

(SEQ ID NO: 6203)
WPPPGTGDVVVQAPTQVPGFLGDSVTLPCYLQVPNMEVTHVSQLTWARH
GESGSMAVFHQTQGPSYSESKRLEFVAARLGAELRNASLRMFGLRVEDE
GNYTCLFVTFPQGSRSVDIWLRVLAKPQNTAEVQKVQLTGEPVPMARCV
STGGRPPAQITWHSDLGGMPNTSQVPGFLSGTVTVTSLWILVPSSQVDG
KNVTCKVEHESFEKPQLLTVNLTVYYPPEVSISGYDNNWYLGQNEATLT
CDARSNPEPTGYNWSTTMGPLPPFAVAQGAQLLIRPVDKPINTTLICNV
TNALGARQAELTVQVKEGPPSEHSGISRN,

a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 6203.

In yet other embodiments, the NK cell engager is a ligand of DAP10, which is an adapter for NKG2D (see e.g., Proc Natl Acad Sci USA. 2005 May 24; 102(21): 7641-7646; and Blood, 15 Sep. 2011 Volume 118, Number 11, the full contents of each of which is incorporated by reference herein).

In other embodiments, the NK cell engager is a ligand of CD16, which is a CD16a/b ligand, e.g., a CD16a/b ligand further comprising an antibody Fc region (see e.g., Front Immunol. 2013; 4: 76 discusses how antibodies use the Fc to trigger NK cells through CD16, the full contents of which are incorporated herein).

In other embodiments, the NK cell engager is a ligand of CRTAM, which is NECL2, e.g., wherein NECL2 comprises the amino acid sequence:

(SEQ ID NO: 6204)
QNLFTKDVTVIEGEVATISCQVNKSDDSVIQLLNPNRQTIYFRDFRPLK
DSRFQLLNFSSSELKVSLTNVSISDEGRYFCQLYTDPPQESYTTITVLV
PPRNLMIDIQKDTAVEGEEIEVNCTAMASKPATTIRWFKGNTELKGKSE
VEEWSDMYTVTSQLMLKVHKEDDGVPVICQVEHPAVTGNLQTQRYLEVQ
YKPQVHIQMTYPLQGLTREGDALELTCEAIGKPQPVMVTWVRVDDEMPQ
HAVLSGPNLFINNLNKTDNGTYRCEASNIVGKAHSDYMLYVYDPPTTIP
PPTTTTTTTTTTTTTILTIITDSRAGEEGSIRAVDH,

a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 6204.

In other embodiments, the NK cell engager is a ligand of CD27, which is CD70, e.g., wherein CD70 comprises the amino acid sequence:

(SEQ ID NO: 6205)
QRFAQAQQQLPLESLGWDVAELQLNHTGPQQDPRLYWQGGPALGRSFLH
GPELDKGQLRIHRDGIYMVHIQVTLAICSSTTASRHHPTTLAVGICSPA
SRSISLLRLSFHQGCTIASQRLTPLARGDTLCTNLTGTLLPSRNTDETF
FGVQWVRP,

a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 6205.

In other embodiments, the NK cell engager is a ligand of PSGL1, which is L-selectin (CD62L), e.g., wherein L-selectin comprises the amino acid sequence:

(SEQ ID NO: 6206)
WTYHYSEKPMNWQRARRFCRDNYTDLVAIQNKAEIEYLEKTLPFSRSYY
WIGIRKIGGIWTWVGTNKSLTEEAENWGDGEPNNKKNKEDCVEIYIKRN
KDAGKWNDDACHKLKAALCYTASCQPWSCSGHGECVEIINNYTCNCDVG
YYGPQCQFVIQCEPLEAPELGTMDCTHPLGNFSFSSQCAFSCSEGTNLT
GIEETTCGPFGNWSSPEPTCQVIQCEPLSAPDLGIMNCSHPLASFSFTS
ACTFICSEGTELIGKKKTICESSGIWSNPSPICQKLDKSFSMIKEGDY
N,

a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 6206.

In other embodiments, the NK cell engager is a ligand of CD96, which is NECL5, e.g., wherein NECL5 comprises the amino acid sequence:

(SEQ ID NO: 6203)
WPPPGTGDVVVQAPTQVPGFLGDSVTLPCYLQVPNMEVTHVSQLTWARH
GESGSMAVFHQTQGPSYSESKRLEFVAARLGAELRNASLRMFGLRVEDE
GNYTCLFVTFPQGSRSVDIWLRVLAKPQNTAEVQKVQLTGEPVPMARCV
STGGRPPAQITWHSDLGGMPNTSQVPGFLSGTVTVTSLWILVPSSQVDG
KNVTCKVEHESFEKPQLLTVNLTVYYPPEVSISGYDNNWYLGQNEATLT
CDARSNPEPTGYNWSTTMGPLPPFAVAQGAQLLIRPVDKPINTTLICNV
TNALGARQAELTVQVKEGPPSEHSGISRN,

a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 6203 or 6204.

In other embodiments, the NK cell engager is a ligand of CD100 (SEMA4D), which is CD72, e.g., wherein CD72 comprises the amino acid sequence:

(SEQ ID NO: 6207)
RYLQVSQQLQQTNRVLEVINSSLRQQLRLKITQLGQSAEDLQGSRRELA
QSQEALQVEQRAHQAAEGQLQACQADRQKTKETLQSEEQQRRALEQKLS
NMENRLKPFFTCGSADTCCPSGWIMHQKSCFYISLTSKNWQESQKQCET
LSSKLATFSEIYPQSHSYYFLNSLLPNGGSGNSYWTGLSSNKDWKLTDD
TQRTRTYAQSSKCNKVHKTWSWWTLESESCRSSLPYICEMTAFRFPD,

a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 6207.

In other embodiments, the NK cell engager is a ligand of NKp80, which is CLEC2B (AICL), e.g., wherein CLEC2B (AICL) comprises the amino acid sequence:

(SEQ ID NO: 6208)
KLTRDSQSLCPYDWIGFQNKCYYFSKEEGDWNSSKYNCSTQHADLTIID
NIEEMNFLRRYKCSSDHWIGLKMAKNRTGQWVDGATFTKSFGMRGSEGC
AYLSDDGAATARCYTERKWICRKRIH,

a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 6208.

In other embodiments, the NK cell engager is a ligand of CD244, which is CD48, e.g., wherein CD48 comprises the amino acid sequence:

(SEQ ID NO: 6209)
QGHLVHMTVVSGSNVTLNISESLPENYKQLTWFYTFDQKIVEWDSRKSK
YFESKFKGRVRLDPQSGALYISKVQKEDNSTYIMRVLKKTGNEQEWKIK
LQVLDPVPKPVIKIEKIEDMDDNCYLKLSCVIPGESVNYTWYGDKRPFP
KELQNSVLETTLMPHNYSRCYTCQVSNSVSSKNGTVCLSPPCTLARS,

a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 6209.

In some embodiments, the NK cell engager is a viral hemagglutinin (HA), HA is a glycoprotein found on the surface of influenza viruses. It is responsible for binding the virus to cells with sialic acid on the membranes, such as cells in the upper respiratory tract or erythrocytes. HA has at least 18 different antigens. These subtypes are named H1 through H18. NCRs can recognize viral proteins. NKp46 has been shown to be able to interact with the HA of influenza and the HA-NA of Paramyxovirus, including Sendai virus and Newcastle disease virus. Besides NKp46, NKp44 can also functionally interact with HA of different influenza subtypes.

In some embodiments of any of the multifunctional molecules described herein, the immune cell engager is an NK cell engager, e.g., an NK cell engager that mediates binding to and activation of an NK cell, or an NK cell engager that mediates binding to but not activation of an NK cell. In certain embodiments, the NK cell engager is chosen from an antibody molecule, e.g., an antigen binding domain, or ligand that binds to (e.g., activates): NKp30, NKp40, NKp44, NKp46, NKG2D, DNAM1, DAP10, CD16 (e.g., CD16a, CD16b, or both), CRTAM, CD27, PSGL1, CD96, CD100 (SEMA4D), NKp80, CD244 (also known as SLAMF4 or 2B4), SLAMF6, SLAMF7, KIR2DS2, KIR2DS4, KIR3DS1, KIR2DS3, KIR2DS5, KIR2DS1, CD94, NKG2C, NKG2E, or CD160, e.g., the NK cell engager is an antibody molecule or ligand that binds to (e.g., activates) NKp30. In certain some embodiments, the NK cell engager is an antibody molecule, e.g., an antigen binding domain.

In some embodiments, the NK cell engager is capable of engaging an NK cell.

In some embodiments, the NK cell engager is an antibody molecule, e.g., an antigen binding domain, that binds to NKp30, NKp46, NKG2D, or CD16.

In some embodiments, the multifunctional molecule:
(i) binds specifically to an epitope of NKp30, NKp46, NKG2D, or CD16, e.g., the same or similar epitope as the epitope recognized by an anti-NKp30, anti-NKp46, anti-NKG2D, or anti-CD16 antibody molecule as described herein;
(ii) shows the same or similar binding affinity or specificity, or both, as an anti-NKp30, anti-NKp46, anti-NKG2D, or anti-CD16 antibody molecule as described herein;
(iii) inhibits, e.g., competitively inhibits, the binding of an anti-NKp30, anti-NKp46, anti-NKG2D, or anti-CD16 antibody molecule as described herein;
(iv) binds the same or an overlapping epitope with an anti-NKp30, anti-NKp46, anti-NKG2D, or anti-CD16 antibody molecule as described herein; or
(v) competes for binding, and/or binds the same epitope, with an anti-NKp30, anti-NKp46, anti-NKG2D, or anti-CD16 molecule as described herein.

In some embodiments, the anti-NKp30, anti-NKp46, anti-NKG2D, or anti-CD16 antibody molecule comprises one or more CDRs, framework regions, variable domains, heavy or light chains, or an antigen binding domain chosen from Tables 7, 8, 35, 36, 9, 10, 15, or 34, or a sequence substantially identical thereto. In some embodiments, the NK cell engager is an antibody molecule, e.g., an antigen binding domain, that binds to NKp30. In some embodiments, lysis of the lymphoma cell or lymphocyte is mediated by NKp30. In some embodiments, the multifunctional molecule does not activate the NK cell when incubated with the NK cell in the absence of the tumor antigen on the lymphoma cell or TRBC1 or TRBC2 on the lymphocyte. In some embodiments, the multifunctional molecule activates the NK cell when the NK cell is a NKp30 expressing NK cell and either: (1) the tumor antigen on the lymphoma cell is also present or (2) TRBC1 or TRBC2 on the lymphocyte is also present. In some embodiments, the multifunctional molecule does not activate the NK cell when the NK cell is not a NKp30 expressing NK cell and either: (1) the tumor antigen on the lymphoma cell is also present or (2) TRBC1 or TRBC2 on the lymphocyte is also present.

In some embodiments, the NK cell engager comprises:
(i) a heavy chain variable region (VH) comprising a heavy chain complementarity determining region 1 (VHCDR1) amino acid sequence of SEQ ID NO: 6000 (or a sequence with no more than 1, 2, 3, or 4 mutations, e.g., substitutions, additions, or deletions), a VHCDR2 amino acid sequence of SEQ ID NO: 6001 (or a sequence with no more than 1, 2, 3, or 4 mutations, e.g., substitutions, additions, or deletions), and/or a VHCDR3 amino acid sequence of SEQ ID NO: 6002 (or a sequence with no more than 1, 2, 3, or 4 mutations, e.g., substitutions, additions, or deletions), and
(ii) a light chain variable region (VL) comprising a light chain complementarity determining region 1 (VLCDR1) amino acid sequence of SEQ ID NO: 6063 (or a sequence with no more than 1, 2, 3, or 4 mutations, e.g., substitutions, additions, or deletions), a VLCDR2 amino acid sequence of SEQ ID NO: 6064 (or a sequence with no more than 1, 2, 3, or 4 mutations, e.g., substitutions, additions, or deletions), and/or a VLCDR3 amino acid sequence of SEQ ID NO: 7293 (or a sequence with no more than 1, 2, 3, or 4 mutations, e.g., substitutions, additions, or deletions).

In some embodiments, the NK cell engager comprises:
(i) a heavy chain variable region (VH) comprising a heavy chain complementarity determining region 1 (VHCDR1) amino acid sequence of SEQ ID NO: 6000, a VHCDR2 amino acid sequence of SEQ ID NO: 6001, and/or a VHCDR3 amino acid sequence of SEQ ID NO: 6002, and
(ii) a light chain variable region (VL) comprising a light chain complementarity determining region 1 (VLCDR1) amino acid sequence of SEQ ID NO: 6063, a VLCDR2 amino acid sequence of SEQ ID NO: 6064, and/or a VLCDR3 amino acid sequence of SEQ ID NO: 7293.

In some embodiments, the NK cell engager comprises: (1) a heavy chain variable region (VH) comprising a heavy chain framework region 1 (VHFWR1) amino acid sequence of SEQ ID NO: 6003 (or a sequence with no more than 1, 2, 3, 4, 5, or 6 mutations, e.g., substitutions, additions, or deletions, therefrom), a VHFWR2 amino acid sequence of SEQ ID NO: 6004 (or a sequence with no more than 1, 2, 3, 4, 5, or 6 mutations, e.g., substitutions, additions, or deletions, therefrom), a VHFWR3 amino acid sequence of SEQ ID NO: 6005 (or a sequence with no more than 1, 2, 3, 4, 5, or 6 mutations, e.g., substitutions, additions, or deletions, therefrom), or a VHFWR4 amino acid sequence of SEQ ID NO: 6006 (or a sequence with no more than 1, 2, 3, 4, 5, or 6 mutations, e.g., substitutions, additions, or deletions, therefrom), and/or (2) a light chain variable region (VL) comprising a light chain framework region 1 (VLFWR1) amino acid sequence of SEQ ID NO: 6066 (or a sequence with no more than 1, 2, 3, 4, 5, or 6 mutations, e.g., substitutions, additions, or deletions, therefrom), a VLFWR2 amino acid sequence of SEQ ID NO: 6067 (or a sequence with no more than 1, 2, 3, 4, 5, or 6 mutations, e.g., substitutions, additions, or deletions, therefrom), a VLFWR3 amino acid sequence of SEQ ID NO: 7292 (or a sequence with no more than 1, 2, 3, 4, 5, or 6 mutations, e.g., substitutions, additions, or deletions, therefrom), or a VLFWR4 amino acid sequence of SEQ ID NO: 6069 (or a sequence with no more than 1, 2, 3, 4, 5, or 6 mutations, e.g., substitutions, additions, or deletions, therefrom).

In some embodiments, the NK cell engager comprises:
(1) a heavy chain variable region (VH) comprising a heavy chain framework region 1 (VHFWR1) amino acid sequence of SEQ ID NO: 6003, a VHFWR2 amino acid sequence of SEQ ID NO: 6004, a VHFWR3 amino acid sequence of SEQ ID NO: 6005, or a VHFWR4 amino acid sequence of SEQ ID NO: 6006, and
(3) a light chain variable region (VL) comprising a light chain framework region 1 (VLFWR1) amino acid sequence of SEQ ID NO: 6066, a VLFWR2 amino acid sequence of SEQ ID NO: 6067, a VLFWR3 amino acid sequence of SEQ ID NO: 7292, or a VLFWR4 amino acid sequence of SEQ ID NO: 6069.

In some embodiments, the NK cell engager comprises:
(i) a VH comprising the amino acid sequence of SEQ ID NO: 6121 (or an amino acid sequence having at least about 75%, 80%, 85%, 90%, 95%, or 99% sequence identity to SEQ ID NO: 6121), and/or
(ii) a VL comprising the amino acid sequence of SEQ ID NO: 7294 (or an amino acid sequence having at least about 93%, 95%, or 99% sequence identity to SEQ ID NO: 7294).

In some embodiments, the NK cell engager comprises a heavy chain comprising the amino acid sequence of SEQ ID NOs: 6148 or 6149 (or an amino acid sequence having at least about 75%, 80%, 85%, 90%, 95%, or 99% sequence identity to SEQ ID NOs: 6148 or 6149).

In some embodiments, the NK cell engager comprises a light chain comprising the amino acid sequence of SEQ ID NO: 6150 (or an amino acid sequence having at least about 75%, 80%, 85%, 90%, 95%, or 99% sequence identity to SEQ ID NO: 6150).

In some embodiments, the NK cell engager comprises a heavy chain comprising the amino acid sequence of SEQ ID NOs: 6148 or 6149 (or an amino acid sequence having at least about 75%, 80%, 85%, 90%, 95%, or 99% sequence identity to SEQ ID NOs: 6148 or 6149), and a light chain comprising the amino acid sequence of SEQ ID NO: 6150 (or an amino acid sequence having at least about 75%, 80%, 85%, 90%, 95%, or 99% sequence identity to SEQ ID NO: 6150).

In some embodiments, the NK cell engager comprises a heavy chain variable region (VH) comprising a heavy chain framework region 1 (VHFWR1) amino acid sequence of SEQ ID NO: 6014 (or a sequence with no more than 1, 2, 3, 4, 5, or 6 mutations, e.g., substitutions, additions, or deletions, therefrom), a VHFWR2 amino acid sequence of SEQ ID NO: 6015 (or a sequence with no more than 1, 2, 3, 4, 5, or 6 mutations, e.g., substitutions, additions, or deletions, therefrom), a VHFWR3 amino acid sequence of SEQ ID NO: 6016 (or a sequence with no more than 1, 2, 3, 4, 5, or 6 mutations, e.g., substitutions, additions, or deletions, therefrom), or a VHFWR4 amino acid sequence of SEQ ID NO: 6017 (or a sequence with no more than 1, 2, 3, 4, 5, or 6 mutations, e.g., substitutions, additions, or deletions, therefrom).

In some embodiments, the NK cell engager comprises a heavy chain variable region (VH) comprising a heavy chain framework region 1 (VHFWR1) amino acid sequence of SEQ ID NO: 6014, a VHFWR2 amino acid sequence of SEQ ID NO: 6015, a VHFWR3 amino acid sequence of SEQ ID NO: 6016, or a VHFWR4 amino acid sequence of SEQ ID NO: 6017.

In some embodiments, the NK cell engager comprises a VH comprising the amino acid sequence of SEQ ID NO: 6123 (or an amino acid sequence having at least about 75%, 80%, 85%, 90%, 95%, or 99% sequence identity to SEQ ID NO: 6123).

In some embodiments, the NK cell engager comprises a heavy chain variable region (VH) comprising a heavy chain framework region 1 (VHFWR1) amino acid sequence of SEQ ID NO: 6018 (or a sequence with no more than 1, 2, 3, 4, 5, or 6 mutations, e.g., substitutions, additions, or deletions, therefrom), a VHFWR2 amino acid sequence of SEQ ID NO: 6019 (or a sequence with no more than 1, 2, 3, 4, 5, or 6 mutations, e.g., substitutions, additions, or deletions, therefrom), a VHFWR3 amino acid sequence of SEQ ID NO: 6020 (or a sequence with no more than 1, 2, 3, 4, 5, or 6 mutations, e.g., substitutions, additions, or deletions, therefrom), or a VHFWR4 amino acid sequence of SEQ ID NO: 6021 (or a sequence with no more than 1, 2, 3, 4, 5, or 6 mutations, e.g., substitutions, additions, or deletions, therefrom).

In some embodiments, the NK cell engager comprises a heavy chain variable region (VH) comprising a heavy chain framework region 1 (VHFWR1) amino acid sequence of SEQ ID NO: 6018, a VHFWR2 amino acid sequence of SEQ ID NO: 6019, a VHFWR3 amino acid sequence of SEQ ID NO: 6020, or a VHFWR4 amino acid sequence of SEQ ID NO: 6021.

In some embodiments, the NK cell engager comprises a VH comprising the amino acid sequence of SEQ ID NO: 6124 (or an amino acid sequence having at least about 75%, 80%, 85%, 90%, 95%, or 99% sequence identity to SEQ ID NO: 6124).

In some embodiments, the NK cell engager comprises a heavy chain variable region (VH) comprising a heavy chain framework region 1 (VHFWR1) amino acid sequence of SEQ ID NO: 6022 (or a sequence with no more than 1, 2, 3, 4, 5, or 6 mutations, e.g., substitutions, additions, or deletions, therefrom), a VHFWR2 amino acid sequence of SEQ ID NO: 6023 (or a sequence with no more than 1, 2, 3, 4, 5, or 6 mutations, e.g., substitutions, additions, or deletions, therefrom), a VHFWR3 amino acid sequence of SEQ ID NO: 6024 (or a sequence with no more than 1, 2, 3, 4, 5, or 6 mutations, e.g., substitutions, additions, or deletions, therefrom), or a VHFWR4 amino acid sequence of SEQ ID NO: 6025 (or a sequence with no more than 1, 2, 3, 4, 5, or 6 mutations, e.g., substitutions, additions, or deletions, therefrom). In certain embodiments, the NK cell engager comprises a heavy chain variable region (VH) comprising a heavy chain framework region 1 (VHFWR1) amino acid sequence of SEQ ID NO: 6022, a VHFWR2 amino acid sequence of SEQ ID NO: 6023, a VHFWR3 amino acid sequence of SEQ ID NO: 6024, or a VHFWR4 amino acid sequence of SEQ ID NO: 6025. In certain embodiments, the NK cell engager comprises a VH comprising the amino acid sequence of SEQ ID NO: 6125 (or an amino acid sequence having at least about 75%, 80%, 85%, 90%, 95%, or 99% sequence identity to SEQ ID NO: 6125).

In some embodiments, the NK cell engager comprises a heavy chain variable region (VH) comprising a heavy chain framework region 1 (VHFWR1) amino acid sequence of SEQ ID NO: 6026 (or a sequence with no more than 1, 2, 3, 4, 5, or 6 mutations, e.g., substitutions, additions, or deletions, therefrom), a VHFWR2 amino acid sequence of SEQ ID NO: 6027 (or a sequence with no more than 1, 2, 3, 4, 5, or 6 mutations, e.g., substitutions, additions, or deletions, therefrom), a VHFWR3 amino acid sequence of SEQ ID NO: 6028 (or a sequence with no more than 1, 2, 3, 4, 5, or 6 mutations, e.g., substitutions, additions, or deletions, therefrom), or a VHFWR4 amino acid sequence of SEQ ID NO: 6029 (or a sequence with no more than 1, 2, 3, 4, 5, or 6 mutations, e.g., substitutions, additions, or deletions, therefrom). In certain embodiments, the NK cell engager comprises a heavy chain variable region (VH) comprising a heavy chain framework region 1 (VHFWR1) amino acid sequence of SEQ ID NO: 6026, a VHFWR2 amino acid sequence of SEQ ID NO: 6027, a VHFWR3 amino acid sequence of SEQ ID NO: 6028, or a VHFWR4 amino acid sequence of SEQ ID NO: 6029. In certain embodiments, the NK cell engager comprises a VH comprising the amino acid sequence of SEQ ID NO: 6126 (or an amino acid sequence having at least about 75%, 80%, 85%, 90%, 95%, or 99% sequence identity to SEQ ID NO: 6126).

In some embodiments, the NK cell engager comprises a heavy chain variable region (VH) comprising a heavy chain framework region 1 (VHFWR1) amino acid sequence of SEQ ID NO: 6030 (or a sequence with no more than 1, 2, 3, 4, 5, or 6 mutations, e.g., substitutions, additions, or deletions, therefrom), a VHFWR2 amino acid sequence of SEQ ID NO: 6032 (or a sequence with no more than 1, 2, 3, 4, 5, or 6 mutations, e.g., substitutions, additions, or deletions, therefrom), a VHFWR3 amino acid sequence of SEQ ID NO: 6033 (or a sequence with no more than 1, 2, 3, 4, 5, or 6 mutations, e.g., substitutions, additions, or deletions, therefrom), or a VHFWR4 amino acid sequence of SEQ ID NO: 6034 (or a sequence with no more than 1, 2, 3, 4, 5, or 6 mutations, e.g., substitutions, additions, or deletions, therefrom). In certain embodiments, the NK cell engager comprises a heavy chain variable region (VH) comprising a heavy chain framework region 1 (VHFWR1) amino acid sequence of SEQ ID NO: 6030, a VHFWR2 amino acid sequence of SEQ ID NO: 6032, a VHFWR3 amino acid sequence of SEQ ID NO: 6033, or a VHFWR4 amino acid sequence of SEQ ID NO: 6034. In certain embodiments, the NK cell engager comprises a VH comprising the amino acid sequence of SEQ ID NO: 6127 (or an amino acid sequence having at least about 75%, 80%, 85%, 90%, 95%, or 99% sequence identity to SEQ ID NO: 6127).

In some embodiments, the NK cell engager comprises a heavy chain variable region (VH) comprising a heavy chain framework region 1 (VHFWR1) amino acid sequence of SEQ ID NO: 6035 (or a sequence with no more than 1, 2, 3, 4, 5, or 6 mutations, e.g., substitutions, additions, or deletions, therefrom), a VHFWR2 amino acid sequence of SEQ ID NO: 6036 (or a sequence with no more than 1, 2, 3, 4, 5, or 6 mutations, e.g., substitutions, additions, or deletions, therefrom), a VHFWR3 amino acid sequence of SEQ ID NO: 6037 (or a sequence with no more than 1, 2, 3, 4, 5, or 6 mutations, e.g., substitutions, additions, or deletions, therefrom), or a VHFWR4 amino acid sequence of SEQ ID NO: 6038 (or a sequence with no more than 1, 2, 3, 4, 5, or 6 mutations, e.g., substitutions, additions, or deletions, therefrom).

In some embodiments, the NK cell engager comprises a heavy chain variable region (VH) comprising a heavy chain framework region 1 (VHFWR1) amino acid sequence of SEQ ID NO: 6035, a VHFWR2 amino acid sequence of SEQ ID NO: 6036, a VHFWR3 amino acid sequence of SEQ ID NO: 6037, or a VHFWR4 amino acid sequence of SEQ ID NO: 6038. In certain embodiments, the NK cell engager comprises a VH comprising the amino acid sequence of SEQ ID NO: 6128 (or an amino acid sequence having at least about 75%, 80%, 85%, 90%, 95%, or 99% sequence identity to SEQ ID NO: 6128).

In some embodiments, the NK cell engager comprises a light chain variable region (VL) comprising a light chain framework region 1 (VLFWR1) amino acid sequence of SEQ ID NO: 6077 (or a sequence with no more than 1, 2, 3, 4, 5, or 6 mutations, e.g., substitutions, additions, or deletions, therefrom), a VLFWR2 amino acid sequence of SEQ ID NO: 6078 (or a sequence with no more than 1, 2, 3, 4, 5, or 6 mutations, e.g., substitutions, additions, or deletions, therefrom), a VLFWR3 amino acid sequence of SEQ ID NO: 6079 (or a sequence with no more than 1, 2, 3, 4, 5, or 6 mutations, e.g., substitutions, additions, or deletions, therefrom), or a VLFWR4 amino acid sequence of SEQ ID NO: 6080 (or a sequence with no more than 1, 2, 3, 4, 5, or 6 mutations, e.g., substitutions, additions, or deletions, therefrom). In certain embodiments, the NK cell engager comprises a light chain variable region (VL) comprising a light chain framework region 1 (VLFWR1) amino acid sequence of SEQ ID NO: 6077, a VLFWR2 amino acid sequence of SEQ ID NO: 6078, a VLFWR3 amino acid sequence of SEQ ID NO: 6079, or a VLFWR4 amino acid sequence of SEQ ID NO: 6080. In certain embodiments, the NK cell engager comprises a VL comprising the amino acid sequence of SEQ ID NO: 6137 (or an amino acid sequence having at least about 93%, 95%, or 99% sequence identity to SEQ ID NO: 6137).

In some embodiments, the NK cell engager comprises a light chain variable region (VL) comprising a light chain framework region 1 (VLFWR1) amino acid sequence of SEQ ID NO: 6081 (or a sequence with no more than 1, 2, 3, 4, 5, or 6 mutations, e.g., substitutions, additions, or deletions, therefrom), a VLFWR2 amino acid sequence of SEQ ID NO: 6082 (or a sequence with no more than 1, 2, 3, 4, 5, or 6 mutations, e.g., substitutions, additions, or deletions, therefrom), a VLFWR3 amino acid sequence of SEQ ID NO: 6083 (or a sequence with no more than 1, 2, 3, 4, 5, or 6 mutations, e.g., substitutions, additions, or deletions, therefrom), or a VLFWR4 amino acid sequence of SEQ ID NO: 6084 (or a sequence with no more than 1, 2, 3, 4, 5, or 6 mutations, e.g., substitutions, additions, or deletions, therefrom).

In some embodiments, the NK cell engager comprises a light chain variable region (VL) comprising a light chain framework region 1 (VLFWR1) amino acid sequence of SEQ ID NO: 6081, a VLFWR2 amino acid sequence of SEQ ID NO: 6082, a VLFWR3 amino acid sequence of SEQ ID NO: 6083, or a VLFWR4 amino acid sequence of SEQ ID NO: 6084. In certain embodiments, the NK cell engager comprises a VL comprising the amino acid sequence of SEQ ID NO: 6138 (or an amino acid sequence having at least about 93%, 95%, or 99% sequence identity to SEQ ID NO: 6138).

In some embodiments, the NK cell engager comprises a light chain variable region (VL) comprising a light chain framework region 1 (VLFWR1) amino acid sequence of SEQ ID NO: 6085 (or a sequence with no more than 1, 2, 3, 4, 5, or 6 mutations, e.g., substitutions, additions, or deletions, therefrom), a VLFWR2 amino acid sequence of SEQ ID NO: 6086 (or a sequence with no more than 1, 2, 3, 4, 5, or 6 mutations, e.g., substitutions, additions, or deletions, therefrom), a VLFWR3 amino acid sequence of SEQ ID NO: 6087 (or a sequence with no more than 1, 2, 3, 4, 5, or 6 mutations, e.g., substitutions, additions, or deletions, therefrom), or a VLFWR4 amino acid sequence of SEQ ID NO: 6088 (or a sequence with no more than 1, 2, 3, 4, 5, or 6 mutations, e.g., substitutions, additions, or deletions, therefrom). In certain embodiments, the NK cell engager comprises a light chain variable region (VL) comprising a light chain framework region 1 (VLFWR1) amino acid sequence of SEQ ID NO: 6085, a VLFWR2 amino acid sequence of SEQ ID NO: 6086, a VLFWR3 amino acid sequence of SEQ ID NO: 6087, or a VLFWR4 amino acid sequence of SEQ ID NO: 6088. In certain embodiments, the NK cell engager comprises a VL comprising the amino acid sequence of SEQ ID NO: 6139 (or an amino acid sequence having at least about 93%, 95%, or 99% sequence identity to SEQ ID NO: 6139).

In some embodiments, the NK cell engager comprises a light chain variable region (VL) comprising a light chain framework region 1 (VLFWR1) amino acid sequence of SEQ ID NO: 6089 (or a sequence with no more than 1, 2, 3, 4, 5, or 6 mutations, e.g., substitutions, additions, or deletions, therefrom), a VLFWR2 amino acid sequence of SEQ ID NO: 6090 (or a sequence with no more than 1, 2, 3, 4, 5, or 6 mutations, e.g., substitutions, additions, or deletions, therefrom), a VLFWR3 amino acid sequence of SEQ ID NO: 6091 (or a sequence with no more than 1, 2, 3, 4, 5, or 6 mutations, e.g., substitutions, additions, or deletions, therefrom), or a VLFWR4 amino acid sequence of SEQ ID NO: 6092 (or a sequence with no more than 1, 2, 3, 4, 5, or 6 mutations, e.g., substitutions, additions, or deletions, therefrom). In certain embodiments, the NK cell engager comprises a light chain variable region (VL) comprising a light chain framework region 1 (VLFWR1) amino acid sequence of SEQ ID NO: 6089, a VLFWR2 amino acid sequence of SEQ ID NO: 6090, a VLFWR3 amino acid sequence of SEQ ID NO: 6091, or a VLFWR4 amino acid sequence of SEQ ID NO: 6092. In certain embodiments, the NK cell engager comprises a VL comprising the amino acid sequence of SEQ ID NO: 6140 (or an amino acid sequence having at least about 93%, 95%, or 99% sequence identity to SEQ ID NO: 6140).

In some embodiments, the NK cell engager comprises a light chain variable region (VL) comprising a light chain framework region 1 (VLFWR1) amino acid sequence of SEQ ID NO: 6093 (or a sequence with no more than 1, 2, 3, 4, 5, or 6 mutations, e.g., substitutions, additions, or deletions, therefrom), a VLFWR2 amino acid sequence of SEQ ID NO: 6094 (or a sequence with no more than 1, 2, 3, 4, 5, or 6 mutations, e.g., substitutions, additions, or deletions, therefrom), a VLFWR3 amino acid sequence of SEQ ID NO: 6095 (or a sequence with no more than 1, 2, 3, 4, 5, or 6 mutations, e.g., substitutions, additions, or deletions, therefrom), or a VLFWR4 amino acid sequence of SEQ ID NO: 6096 (or a sequence with no more than 1, 2, 3, 4, 5, or 6 mutations, e.g., substitutions, additions, or deletions, therefrom). In certain embodiments, the NK cell engager comprises a light chain variable region (VL) comprising a light chain framework region 1 (VLFWR1) amino acid sequence of SEQ ID NO: 6093, a VLFWR2 amino acid sequence of SEQ ID NO: 6094, a VLFWR3 amino acid sequence of SEQ ID NO: 6095, or a VLFWR4 amino acid sequence of SEQ ID NO: 6096. In certain embodiments, the NK cell engager comprises a VL comprising the amino acid sequence of SEQ ID NO: 6141 (or an amino acid sequence having at least about 93%, 95%, or 99% sequence identity to SEQ ID NO: 6141).

In some embodiments, the NK cell engager comprises:
(i) a heavy chain variable region (VH) comprising a heavy chain complementarity determining region 1 (VHCDR1) amino acid sequence of SEQ ID NO: 6007 (or a sequence with no more than 1, 2, 3, or 4 mutations, e.g., substitutions, additions, or deletions), a VHCDR2 amino acid sequence of SEQ ID NO: 6008 (or a sequence with no more than 1, 2, 3, or 4 mutations, e.g., substitutions, additions, or deletions), and/or a VHCDR3 amino acid sequence of SEQ ID NO: 6009 (or a sequence with no more than 1, 2, 3, or 4 mutations, e.g., substitutions, additions, or deletions), and
(ii) a light chain variable region (VL) comprising a light chain complementarity determining region 1 (VLCDR1) amino acid sequence of SEQ ID NO: 6070 (or a sequence with no more than 1, 2, 3, or 4 mutations, e.g., substitutions, additions, or deletions), a VLCDR2 amino acid sequence of SEQ ID NO: 6071 (or a sequence with no more than 1, 2, 3, or 4 mutations, e.g., substitutions, additions, or deletions), and/or a VLCDR3 amino acid sequence of SEQ ID NO: 6072 (or a sequence with no more than 1, 2, 3, or 4 mutations, e.g., substitutions, additions, or deletions). In certain embodiments, the NK cell engager comprises:
(i) a heavy chain variable region (VH) comprising a heavy chain complementarity determining region 1 (VHCDR1) amino acid sequence of SEQ ID NO: 6007, a VHCDR2 amino acid sequence of SEQ ID NO: 6008, and/or a VHCDR3 amino acid sequence of SEQ ID NO: 6009, and
(ii) a light chain variable region (VL) comprising a light chain complementarity determining region 1 (VLCDR1) amino acid sequence of SEQ ID NO: 6070, a VLCDR2 amino acid sequence of SEQ ID NO: 6071, and/or a VLCDR3 amino acid sequence of SEQ ID NO: 6072.

In some embodiments, the NK cell engager comprises: (1) a heavy chain variable region (VH) comprising a heavy chain framework region 1 (VHFWR1) amino acid sequence of SEQ ID NO: 6010 (or a sequence with no more than 1, 2, 3, 4, 5, or 6 mutations, e.g., substitutions, additions, or deletions, therefrom), a VHFWR2 amino acid sequence of SEQ ID NO: 6011 (or a sequence with no more than 1, 2, 3, 4, 5, or 6 mutations, e.g., substitutions, additions, or deletions, therefrom), a VHFWR3 amino acid sequence of SEQ ID NO: 6012 (or a sequence with no more than 1, 2, 3, 4, 5, or 6 mutations, e.g., substitutions, additions, or deletions, therefrom), or a VHFWR4 amino acid sequence of SEQ ID NO: 6013 (or a sequence with no more than 1, 2, 3, 4, 5, or 6 mutations, e.g., substitutions, additions, or deletions, therefrom), and/or (2) a light chain variable region (VL) comprising a light chain framework region 1 (VLFWR1) amino acid sequence of SEQ ID NO: 6073 (or a sequence with no more than 1, 2, 3, 4, 5, or 6 mutations, e.g., substitutions, additions, or deletions, therefrom), a VLFWR2 amino acid sequence of SEQ ID NO: 6074 (or a sequence with no more than 1, 2, 3, 4, 5, or 6 mutations, e.g., substitutions, additions, or deletions, therefrom), a VLFWR3 amino acid sequence of SEQ ID NO: 6075 (or a sequence with no more than 1, 2, 3, 4, 5, or 6 mutations, e.g., substitutions, additions, or deletions, therefrom), or a VLFWR4 amino acid sequence of SEQ ID NO: 6076 (or a sequence with no more than 1, 2, 3, 4, 5, or 6 mutations, e.g., substitutions, additions, or deletions, therefrom). In certain embodiments, the NK cell engager comprises: (1) a heavy chain variable region (VH) comprising a heavy chain framework region 1 (VHFWR1) amino acid sequence of SEQ ID NO: 6010, a VHFWR2 amino acid sequence of SEQ ID NO: 6011, a VHFWR3 amino acid sequence of SEQ ID NO: 6012, or a VHFWR4 amino acid sequence of SEQ ID NO: 6013, and (3) a light chain variable region (VL) comprising a light chain framework region 1 (VLFWR1) amino acid sequence of SEQ ID NO: 6073, a VLFWR2 amino acid sequence of SEQ ID NO: 6074, a VLFWR3 amino acid sequence of SEQ ID NO: 6075, or a VLFWR4 amino acid sequence of SEQ ID NO: 6076.

In some embodiments, the NK cell engager comprises:
(i) a VH comprising the amino acid sequence of SEQ ID NO: 6122 (or an amino acid sequence having at least about 75%, 80%, 85%, 90%, 95%, or 99% sequence identity to SEQ ID NO: 6122), and/or
(ii) a VL comprising the amino acid sequence of SEQ ID NO: 6136 (or an amino acid sequence having at least about 93%, 95%, or 99% sequence identity to SEQ ID NO: 6136).

In some embodiments, the NK cell engager comprises a heavy chain comprising the amino acid sequence of SEQ ID NOs: 6151 or 6152 (or an amino acid sequence having at least about 75%, 80%, 85%, 90%, 95%, or 99% sequence identity to SEQ ID NOs: 6151 or 6152).

In some embodiments, the NK cell engager comprises a light chain comprising the amino acid sequence of SEQ ID NO: 6153 (or an amino acid sequence having at least about 75%, 80%, 85%, 90%, 95%, or 99% sequence identity to SEQ ID NO: 6153).

In some embodiments, the NK cell engager comprises a heavy chain comprising the amino acid sequence of SEQ ID NOs: 6151 or 6152 (or an amino acid sequence having at least about 75%, 80%, 85%, 90%, 95%, or 99% sequence identity to SEQ ID NOs: 6151 or 6152), and a light chain comprising the amino acid sequence of SEQ ID NO: 6153 (or an amino acid sequence having at least about 75%, 80%, 85%, 90%, 95%, or 99% sequence identity to SEQ ID NO: 6153).

In some embodiments, the NK cell engager comprises a heavy chain variable region (VH) comprising a heavy chain framework region 1 (VHFWR1) amino acid sequence of SEQ ID NO: 6039 (or a sequence with no more than 1, 2, 3, 4, 5, or 6 mutations, e.g., substitutions, additions, or deletions, therefrom), a VHFWR2 amino acid sequence of SEQ ID NO: 6040 (or a sequence with no more than 1, 2, 3, 4, 5, or 6 mutations, e.g., substitutions, additions, or deletions, therefrom), a VHFWR3 amino acid sequence of SEQ ID NO: 6041 (or a sequence with no more than 1, 2, 3, 4, 5, or 6 mutations, e.g., substitutions, additions, or deletions, therefrom), or a VHFWR4 amino acid sequence of SEQ ID NO: 6042 (or a sequence with no more than 1, 2, 3, 4, 5, or 6 mutations, e.g., substitutions, additions, or deletions, therefrom).

In some embodiments, the NK cell engager comprises a heavy chain variable region (VH) comprising a heavy chain framework region 1 (VHFWR1) amino acid sequence of SEQ ID NO: 6039, a VHFWR2 amino acid sequence of SEQ ID NO: 6040, a VHFWR3 amino acid sequence of SEQ ID NO: 6041, or a VHFWR4 amino acid sequence of SEQ ID NO: 6042. In certain embodiments, the NK cell engager comprises a VH comprising the amino acid sequence of SEQ ID NO: 6129 (or an amino acid sequence having at least about 75%, 80%, 85%, 90%, 95%, or 99% sequence identity to SEQ ID NO: 6129).

In some embodiments, the NK cell engager comprises a heavy chain variable region (VH) comprising a heavy chain framework region 1 (VHFWR1) amino acid sequence of SEQ ID NO: 6043 (or a sequence with no more than 1, 2, 3, 4, 5, or 6 mutations, e.g., substitutions, additions, or deletions, therefrom), a VHFWR2 amino acid sequence of SEQ ID NO: 6044 (or a sequence with no more than 1, 2, 3, 4, 5, or 6 mutations, e.g., substitutions, additions, or deletions, therefrom), a VHFWR3 amino acid sequence of SEQ ID NO: 6045 (or a sequence with no more than 1, 2, 3, 4, 5, or 6 mutations, e.g., substitutions, additions, or deletions, therefrom), or a VHFWR4 amino acid sequence of SEQ ID NO: 6046 (or a sequence with no more than 1, 2, 3, 4, 5, or 6 mutations, e.g., substitutions, additions, or deletions, therefrom).

In some embodiments, the NK cell engager comprises a heavy chain variable region (VH) comprising a heavy chain framework region 1 (VHFWR1) amino acid sequence of SEQ ID NO: 6043, a VHFWR2 amino acid sequence of SEQ ID NO: 6044, a VHFWR3 amino acid sequence of SEQ ID NO: 6045, or a VHFWR4 amino acid sequence of SEQ ID NO: 6046.

In some embodiments, the NK cell engager comprises a VH comprising the amino acid sequence of SEQ ID NO: 6130 (or an amino acid sequence having at least about 75%, 80%, 85%, 90%, 95%, or 99% sequence identity to SEQ ID NO: 6130).

In some embodiments, the NK cell engager comprises a heavy chain variable region (VH) comprising a heavy chain framework region 1 (VHFWR1) amino acid sequence of SEQ ID NO: 6047 (or a sequence with no more than 1, 2, 3, 4, 5, or 6 mutations, e.g., substitutions, additions, or deletions, therefrom), a VHFWR2 amino acid sequence of SEQ ID NO: 6048 (or a sequence with no more than 1, 2, 3, 4, 5, or 6 mutations, e.g., substitutions, additions, or deletions, therefrom), a VHFWR3 amino acid sequence of SEQ ID NO: 6049 (or a sequence with no more than 1, 2, 3, 4, 5, or 6 mutations, e.g., substitutions, additions, or deletions, therefrom), or a VHFWR4 amino acid sequence of SEQ ID NO: 6050 (or a sequence with no more than 1, 2, 3, 4, 5, or 6 mutations, e.g., substitutions, additions, or deletions, therefrom). In certain embodiments, the NK cell engager comprises a heavy chain variable region (VH) comprising a heavy chain framework region 1 (VHFWR1) amino acid sequence of SEQ ID NO: 6047, a VHFWR2 amino acid sequence of SEQ ID NO: 6048, a VHFWR3 amino acid sequence of SEQ ID NO: 6049, or a VHFWR4 amino acid sequence of SEQ ID NO: 6050. In certain embodiments, the NK cell engager comprises a VH comprising the amino acid sequence of SEQ ID NO: 6131 (or an amino acid sequence having at least about 75%, 80%, 85%, 90%, 95%, or 99% sequence identity to SEQ ID NO: 6131).

In some embodiments, the NK cell engager comprises a heavy chain variable region (VH) comprising a heavy chain framework region 1 (VHFWR1) amino acid sequence of SEQ ID NO: 6051 (or a sequence with no more than 1, 2, 3, 4, 5, or 6 mutations, e.g., substitutions, additions, or deletions, therefrom), a VHFWR2 amino acid sequence of SEQ ID NO: 6052 (or a sequence with no more than 1, 2, 3, 4, 5, or 6 mutations, e.g., substitutions, additions, or deletions, therefrom), a VHFWR3 amino acid sequence of SEQ ID NO: 6053 (or a sequence with no more than 1, 2, 3, 4, 5, or 6 mutations, e.g., substitutions, additions, or deletions, therefrom), or a VHFWR4 amino acid sequence of SEQ ID NO: 6054 (or a sequence with no more than 1, 2, 3, 4, 5, or 6 mutations, e.g., substitutions, additions, or deletions, therefrom). In certain embodiments, the NK cell engager comprises a heavy chain variable region (VH) comprising a heavy chain framework region 1 (VHFWR1) amino acid sequence of SEQ ID NO: 6051, a VHFWR2 amino acid sequence of SEQ ID NO: 6052, a VHFWR3 amino acid sequence of SEQ ID NO: 6053, or a VHFWR4 amino acid sequence of SEQ ID NO: 6054. In certain embodiments, the NK cell engager comprises a VH comprising the amino acid sequence of SEQ ID NO: 6132 (or an amino acid sequence having at least about 75%, 80%, 85%, 90%, 95%, or 99% sequence identity to SEQ ID NO: 6132).

In some embodiments, the NK cell engager comprises a heavy chain variable region (VH) comprising a heavy chain framework region 1 (VHFWR1) amino acid sequence of SEQ ID NO: 6055 (or a sequence with no more than 1, 2, 3, 4, 5, or 6 mutations, e.g., substitutions, additions, or deletions, therefrom), a VHFWR2 amino acid sequence of SEQ ID NO: 6056 (or a sequence with no more than 1, 2, 3, 4, 5, or 6 mutations, e.g., substitutions, additions, or deletions, therefrom), a VHFWR3 amino acid sequence of SEQ ID NO: 6057 (or a sequence with no more than 1, 2, 3, 4, 5, or 6 mutations, e.g., substitutions, additions, or deletions, therefrom), or a VHFWR4 amino acid sequence of SEQ ID NO: 6058 (or a sequence with no more than 1, 2, 3, 4, 5, or 6 mutations, e.g., substitutions, additions, or deletions, therefrom). In certain embodiments, the NK cell engager comprises a heavy chain variable region (VH) comprising a heavy chain framework region 1 (VHFWR1) amino acid sequence of SEQ ID NO: 6055, a VHFWR2 amino acid sequence of SEQ ID NO: 6056, a VHFWR3 amino acid sequence of SEQ ID NO: 6057, or a VHFWR4 amino acid sequence of SEQ ID NO: 6058. In certain embodiments, the NK cell engager comprises a VH comprising the amino acid sequence of SEQ ID NO: 6133 (or an amino acid sequence having at least about 75%, 80%, 85%, 90%, 95%, or 99% sequence identity to SEQ ID NO: 6133).

In some embodiments, the NK cell engager comprises a heavy chain variable region (VH) comprising a heavy chain framework region 1 (VHFWR1) amino acid sequence of SEQ ID NO: 6059 (or a sequence with no more than 1, 2, 3, 4, 5, or 6 mutations, e.g., substitutions, additions, or deletions, therefrom), a VHFWR2 amino acid sequence of SEQ ID NO: 6060 (or a sequence with no more than 1, 2, 3, 4, 5, or 6 mutations, e.g., substitutions, additions, or deletions, therefrom), a VHFWR3 amino acid sequence of SEQ ID NO: 6061 (or a sequence with no more than 1, 2, 3, 4, 5, or 6 mutations, e.g., substitutions, additions, or deletions, therefrom), or a VHFWR4 amino acid sequence of SEQ ID NO: 6062 (or a sequence with no more than 1, 2, 3, 4, 5, or 6 mutations, e.g., substitutions, additions, or deletions, therefrom). In certain embodiments, the NK cell engager comprises a heavy chain variable region (VH) comprising a heavy chain framework region 1 (VHFWR1) amino acid sequence of SEQ ID NO: 6059, a VHFWR2 amino acid sequence of SEQ ID NO: 6060, a VHFWR3 amino acid sequence of SEQ ID NO: 6061, or a VHFWR4 amino acid sequence of SEQ ID NO: 6062. In certain embodiments, the NK cell engager comprises a VH comprising the amino acid sequence of SEQ ID NO: 6134 (or an amino acid sequence having at least about 75%, 80%, 85%, 90%, 95%, or 99% sequence identity to SEQ ID NO: 6134).

In some embodiments, wherein the NK cell engager comprises a light chain variable region (VL) comprising a light chain framework region 1 (VLFWR1) amino acid sequence of SEQ ID NO: 6097 (or a sequence with no more than 1, 2, 3, 4, 5, or 6 mutations, e.g., substitutions, additions, or deletions, therefrom), a VLFWR2 amino acid sequence of SEQ ID NO: 6098 (or a sequence with no more than 1, 2, 3, 4, 5, or 6 mutations, e.g., substitutions, additions, or deletions, therefrom), a VLFWR3 amino acid sequence of SEQ ID NO: 6099 (or a sequence with no more than 1, 2, 3, 4, 5, or 6 mutations, e.g., substitutions, additions, or deletions, therefrom), or a VLFWR4 amino acid sequence of SEQ ID NO: 6100 (or a sequence with no more than 1, 2, 3, 4, 5, or 6 mutations, e.g., substitutions, additions, or deletions, therefrom). In certain embodiments, the NK cell engager comprises a light chain variable region (VL) comprising a light chain framework region 1 (VLFWR1) amino acid sequence of SEQ ID NO: 6097, a VLFWR2 amino acid sequence of SEQ ID NO: 6098, a VLFWR3 amino acid sequence of SEQ ID NO: 6099, or a VLFWR4 amino acid sequence of SEQ ID NO: 6100. In certain embodiments, wherein the NK cell engager comprises a VL comprising the amino acid sequence of SEQ ID NO: 6142 (or an amino acid sequence having at least about 93%, 95%, or 99% sequence identity to SEQ ID NO: 6142).

In some embodiments, the NK cell engager comprises a light chain variable region (VL) comprising a light chain framework region 1 (VLFWR1) amino acid sequence of SEQ ID NO: 6101 (or a sequence with no more than 1, 2, 3, 4, 5, or 6 mutations, e.g., substitutions, additions, or deletions, therefrom), a VLFWR2 amino acid sequence of SEQ ID NO: 6102 (or a sequence with no more than 1, 2, 3, 4, 5, or 6 mutations, e.g., substitutions, additions, or deletions, therefrom), a VLFWR3 amino acid sequence of SEQ ID NO: 6103 (or a sequence with no more than 1, 2, 3, 4, 5, or 6 mutations, e.g., substitutions, additions, or deletions, therefrom), or a VLFWR4 amino acid sequence of SEQ ID NO: 6104 (or a sequence with no more than 1, 2, 3, 4, 5, or 6 mutations, e.g., substitutions, additions, or deletions, therefrom). In certain embodiments, the NK cell engager comprises a light chain variable region (VL) comprising a light chain framework region 1 (VLFWR1) amino acid sequence of SEQ ID NO: 6101, a VLFWR2 amino acid sequence of SEQ ID NO: 6102, a VLFWR3 amino acid sequence of SEQ ID NO: 6103, or a VLFWR4 amino acid sequence of SEQ ID NO: 6104. In certain embodiments, the NK cell engager comprises a VL comprising the amino acid sequence of SEQ ID NO: 6143 (or an amino acid sequence having at least about 93%, 95%, or 99% sequence identity to SEQ ID NO: 6143).

In some embodiments, the NK cell engager comprises a light chain variable region (VL) comprising a light chain framework region 1 (VLFWR1) amino acid sequence of SEQ ID NO: 6105 (or a sequence with no more than 1, 2, 3, 4, 5, or 6 mutations, e.g., substitutions, additions, or deletions, therefrom), a VLFWR2 amino acid sequence of SEQ ID NO: 6106 (or a sequence with no more than 1, 2, 3, 4, 5, or 6 mutations, e.g., substitutions, additions, or deletions, therefrom), a VLFWR3 amino acid sequence of SEQ ID NO: 6107 (or a sequence with no more than 1, 2, 3, 4, 5, or 6 mutations, e.g., substitutions, additions, or deletions, therefrom), or a VLFWR4 amino acid sequence of SEQ ID NO: 6108 (or a sequence with no more than 1, 2, 3, 4, 5, or 6 mutations, e.g., substitutions, additions, or deletions, therefrom). In certain embodiments, the NK cell engager comprises a light chain variable region (VL) comprising a light chain framework region 1 (VLFWR1) amino acid sequence of SEQ ID NO: 6105, a VLFWR2 amino acid sequence of SEQ ID NO: 6106, a VLFWR3 amino acid sequence of SEQ ID NO: 6107, or a VLFWR4 amino acid sequence of SEQ ID NO: 6108. In certain embodiments, the NK cell engager comprises a VL comprising the amino acid sequence of SEQ ID NO: 6144 (or an amino acid sequence having at least about 93%, 95%, or 99% sequence identity to SEQ ID NO: 6144).

In some embodiments, the NK cell engager comprises a light chain variable region (VL) comprising a light chain framework region 1 (VLFWR1) amino acid sequence of SEQ ID NO: 6109 (or a sequence with no more than 1, 2, 3, 4, 5, or 6 mutations, e.g., substitutions, additions, or deletions, therefrom), a VLFWR2 amino acid sequence of SEQ ID NO: 6110 (or a sequence with no more than 1, 2, 3, 4, 5, or 6 mutations, e.g., substitutions, additions, or deletions, therefrom), a VLFWR3 amino acid sequence of SEQ ID NO: 6111 (or a sequence with no more than 1, 2, 3, 4, 5, or 6 mutations, e.g., substitutions, additions, or deletions, therefrom), or a VLFWR4 amino acid sequence of SEQ ID NO: 6112 (or a sequence with no more than 1, 2, 3, 4, 5, or 6 mutations, e.g., substitutions, additions, or deletions, therefrom). In certain embodiments, the NK cell engager comprises a light chain variable region (VL) comprising a light chain framework region 1 (VLFWR1) amino acid sequence of SEQ ID NO: 6109, a VLFWR2 amino acid sequence of SEQ ID NO: 6110, a VLFWR3 amino acid sequence of SEQ ID NO: 6111, or a VLFWR4 amino acid sequence of SEQ ID NO: 6112. In certain embodiments, the NK cell engager comprises a VL comprising the amino acid sequence of SEQ ID NO: 6145 (or an amino acid sequence having at least about 93%, 95%, or 99% sequence identity to SEQ ID NO: 6145).

In some embodiments, the NK cell engager comprises a light chain variable region (VL) comprising a light chain framework region 1 (VLFWR1) amino acid sequence of SEQ ID NO: 6113 (or a sequence with no more than 1, 2, 3, 4, 5, or 6 mutations, e.g., substitutions, additions, or deletions, therefrom), a VLFWR2 amino acid sequence of SEQ ID NO: 6114 (or a sequence with no more than 1, 2, 3, 4, 5, or 6 mutations, e.g., substitutions, additions, or deletions, therefrom), a VLFWR3 amino acid sequence of SEQ ID NO: 6115 (or a sequence with no more than 1, 2, 3, 4, 5, or 6 mutations, e.g., substitutions, additions, or deletions, therefrom), or a VLFWR4 amino acid sequence of SEQ ID NO: 6116 (or a sequence with no more than 1, 2, 3, 4, 5, or 6 mutations, e.g., substitutions, additions, or deletions, therefrom). In certain embodiments, the NK cell engager comprises a light chain variable region (VL) comprising a light chain framework region 1 (VLFWR1) amino acid sequence of SEQ ID NO: 6113, a VLFWR2 amino acid sequence of SEQ ID NO: 6114, a VLFWR3 amino acid sequence of SEQ ID NO: 6115, or a VLFWR4 amino acid sequence of SEQ ID NO: 6116. In certain embodiments, the NK cell engager comprises a VL comprising the amino acid sequence of SEQ ID NO: 6146 (or an amino acid sequence having at least about 93%, 95%, or 99% sequence identity to SEQ ID NO: 6146).

In some embodiments, the NK cell engager comprises a light chain variable region (VL) comprising a light chain framework region 1 (VLFWR1) amino acid sequence of SEQ ID NO: 6117 (or a sequence with no more than 1, 2, 3, 4, 5, or 6 mutations, e.g., substitutions, additions, or deletions, therefrom), a VLFWR2 amino acid sequence of SEQ ID NO: 6118 (or a sequence with no more than 1, 2, 3, 4, 5, or 6 mutations, e.g., substitutions, additions, or deletions, therefrom), a VLFWR3 amino acid sequence of SEQ ID NO: 6119 (or a sequence with no more than 1, 2, 3, 4, 5, or 6 mutations, e.g., substitutions, additions, or deletions, therefrom), or a VLFWR4 amino acid sequence of SEQ ID NO: 6120 (or a sequence with no more than 1, 2, 3, 4, 5, or 6 mutations, e.g., substitutions, additions, or deletions, therefrom). In certain embodiments, the NK cell engager comprises a light chain variable region (VL) comprising a light chain framework region 1 (VLFWR1) amino acid sequence of SEQ ID NO: 6117, a VLFWR2 amino acid sequence of SEQ ID NO: 6118, a VLFWR3 amino acid sequence of SEQ ID NO: 6119, or a VLFWR4 amino acid sequence of SEQ ID NO: 6120. In certain embodiments, the NK cell engager comprises a VL comprising the amino acid sequence of SEQ ID NO: 6147 (or an amino acid sequence having at least about 93%, 95%, or 99% sequence identity to SEQ ID NO: 6147).

In some embodiments, the NK cell engager is an antibody molecule, e.g., an antigen binding domain, that binds to NKp46. In certain embodiments, lysis of the lymphoma cell is mediated by NKp46. In some embodiments, the multifunctional molecule does not activate the NK cell when incubated with the NK cell in the absence of the tumor antigen on the lymphoma cell. In some embodiments, the multifunctional molecule activates the NK cell when the NK cell is a NKp46 expressing NK cell and the tumor antigen on the lymphoma cell is also present. In some embodiments, the multifunctional molecule does not activate the NK cell when the NK cell is not a NKp46 expressing NK cell and the tumor antigen on the lymphoma cell is also present. In some embodiments, the NK cell engager comprises a VH comprising the amino acid sequence of SEQ ID NO: 6182 (or an amino acid sequence having at least about 75%, 80%, 85%, 90%, 95%, or 99% sequence identity to SEQ ID NO: 6182). In some embodiments, the NK cell engager comprises a VL comprising the amino acid sequence of SEQ ID NO: 6183 (or an amino acid sequence having at least about 93%, 95%, or 99% sequence identity to SEQ ID NO: 6183). In some embodiments, the NK cell engager comprises an scFv comprising the amino acid sequence of SEQ ID NO: 6181 (or an amino acid sequence having at least about 93%, 95%, or 99% sequence identity to SEQ ID NO: 6181).

In some embodiments, the NK cell engager is an antibody molecule, e.g., an antigen binding domain, that binds to NKG2D. In certain embodiments, lysis of the lymphoma cell is mediated by NKG2D. In some embodiments, the multifunctional molecule does not activate the NK cell when incubated with the NK cell in the absence of the tumor antigen on the lymphoma cell. In some embodiments, the multifunctional molecule activates the NK cell when the NK cell is a NKG2D expressing NK cell and the tumor antigen on the lymphoma cell is also present. In some embodiments, the multifunctional molecule does not activate the NK cell when the NK cell is not a NKG2D expressing NK cell and the tumor antigen on the lymphoma cell is also present. In some embodiments, the NK cell engager comprises a VH comprising the amino acid sequence of SEQ ID NO: 6176 (or an amino acid sequence having at least about 75%, 80%, 85%, 90%, 95%, or 99% sequence identity to SEQ ID NO: 6176). In some embodiments, the NK cell engager comprises a VL comprising the amino acid sequence of SEQ ID NO: 6177 (or an amino acid sequence having at least about 93%, 95%, or 99% sequence identity to SEQ ID NO: 6177). In some embodiments, the NK cell engager comprises an scFv comprising the amino acid sequence of SEQ ID NO: 6175 (or an amino acid sequence having at least about 93%, 95%, or 99% sequence identity to SEQ ID NO: 6175). In some embodiments, the NK cell engager comprises a VH comprising the amino acid sequence of SEQ ID NO: 6179 (or an amino acid sequence having at least about 75%, 80%, 85%, 90%, 95%, or 99% sequence identity to SEQ ID NO: 6179). In some embodiments, the NK cell engager comprises a VL comprising the amino acid sequence of SEQ ID NO: 6180 (or an amino acid sequence having at least about 93%, 95%, or 99% sequence identity to SEQ ID NO: 6180). In some embodiments, the NK cell engager comprises an scFv comprising the amino acid sequence of SEQ ID NO: 6178 (or an amino acid sequence having at least about 93%, 95%, or 99% sequence identity to SEQ ID NO: 6178).

In some embodiments, the NK cell engager is an antibody molecule, e.g., an antigen binding domain, that binds to CD16. In some embodiments, lysis of the lymphoma cell is mediated by CD16. In some embodiments, the multifunctional molecule does not activate the NK cell when incubated with the NK cell in the absence of the tumor antigen on the lymphoma cell. In some embodiments, the multifunctional molecule activates the NK cell when the NK cell is a CD16 expressing NK cell and the tumor antigen on the lymphoma cell is also present. In some embodiments, the multifunctional molecule does not activate the NK cell when the NK cell is not a CD16 expressing NK cell and the tumor antigen on the lymphoma cell is also present. In some embodiments, the NK cell engager comprises a VH comprising the amino acid sequence of SEQ ID NO: 6185 (or an amino acid sequence having at least about 75%, 80%, 85%, 90%, 95%, or 99% sequence identity to SEQ ID NO: 6185). In some embodiments, the NK cell engager comprises a VL comprising the amino acid sequence of SEQ ID NO: 6186 (or an amino acid sequence having at least about 93%, 95%, or 99% sequence identity to SEQ ID NO: 6186). In some embodiments, the NK cell engager comprises an scFv comprising the amino acid sequence of SEQ ID NO: 6184 (or an amino acid sequence having at least about 93%, 95%, or 99% sequence identity to SEQ ID NO: 6184).

In some embodiments, the NK cell engager is a ligand, optionally, the ligand further comprises an immunoglobulin constant region, e.g., an Fc region. In certain embodiments, the NK cell engager is a ligand of NKp44 or NKp46, e.g., a viral HA. In certain embodiments, the NK cell engager is a ligand of DAP10, e.g., a coreceptor for NKG2D. In certain embodiments, the NK cell engager is a ligand of CD16, e.g., a CD16a/b ligand, e.g., a CD16a/b ligand further comprising an antibody Fc region.

### B Cell, Macrophage & Dendritic Cell Engagers

Broadly, B cells, also known as B lymphocytes, are a type of white blood cell of the lymphocyte subtype. They function in the humoral immunity component of the adaptive immune system by secreting antibodies. Additionally, B cells present antigen (they are also classified as professional antigen-presenting cells (APCs)) and secrete cytokines. Macrophages are a type of white blood cell that engulfs and digests cellular debris, foreign substances, microbes, cancer cells via phagocytosis. Besides phagocytosis, they play important roles in nonspecific defense (innate immunity) and also help initiate specific defense mechanisms (adaptive immunity) by recruiting other immune cells such as lymphocytes. For example, they are important as antigen presenters to T cells. Beyond increasing inflammation and stimulating the immune system, macrophages also play an important anti-inflammatory role and can decrease immune reactions through the release of cytokines. Dendritic cells (DCs) are antigen-presenting cells that function in processing antigen material and present it on the cell surface to the T cells of the immune system.

The present disclosure provides, inter alia, multispecific (e.g., bi-, tri-, quad-specific) or multifunctional molecules, that include, e.g., are engineered to contain, one or more B cell, macrophage, and/or dendritic cell engager that mediate binding to and/or activation of a B cell, macrophage, and/or dendritic cell.

Accordingly, in some embodiments, the immune cell engager comprises a B cell, macrophage, and/or dendritic cell engager chosen from one or more of CD40 ligand (CD40L) or a CD70 ligand; an antibody molecule that binds to CD40 or CD70; an antibody molecule to OX40; an OX40 ligand (OX40L); an agonist of a Toll-like receptor (e.g., as described herein, e.g., a TLR4, e.g., a constitutively active TLR4 (caTLR4), or a TLR9 agonists); a 41BB; a CD2; a CD47; or a STING agonist, or a combination thereof.

In some embodiments, the B cell engager is a CD40L, an OX40L, or a CD70 ligand, or an antibody molecule that binds to OX40, CD40 or CD70.

In some embodiments, the macrophage engager is a CD2 agonist. In some embodiments, the macrophage engager is an antigen binding domain that binds to: CD40L or antigen binding domain or ligand that binds CD40, a Toll like receptor (TLR) agonist (e.g., as described herein), e.g., a TLR9 or TLR4 (e.g., caTLR4 (constitutively active TLR4), CD47, or a STING agonist. In some embodiments, the STING agonist is a cyclic dinucleotide, e.g., cyclic di-GMP (cdGMP) or cyclic di-AMP (cdAMP). In some embodiments, the STING agonist is biotinylated.

In some embodiments, the dendritic cell engager is a CD2 agonist. In some embodiments, the dendritic cell engager is a ligand, a receptor agonist, or an antibody molecule that binds to one or more of: OX40L, 41BB, a TLR agonist (e.g., as described herein) (e.g., TLR9 agonist, TLR4 (e.g., caTLR4 (constitutively active TLR4)), CD47, or and a STING agonist. In some embodiments, the STING agonist is a cyclic dinucleotide, e.g., cyclic di-GMP (cdGMP) or cyclic di-AMP (cdAMP). In some embodiments, the STING agonist is biotinylated.

In other embodiments, the immune cell engager mediates binding to, or activation of, one or more of a B cell, a macrophage, and/or a dendritic cell. Exemplary B cell, macrophage, and/or dendritic cell engagers can be chosen from one or more of CD40 ligand (CD40L) or a CD70 ligand; an antibody molecule that binds to CD40 or CD70; an antibody molecule to OX40; an OX40 ligand (OX40L); a Toll-like receptor agonist (e.g., a TLR4, e.g., a constitutively active TLR4 (caTLR4) or a TLR9 agonist); a 41BB agonist; a CD2; a CD47; or a STING agonist, or a combination thereof.

In some embodiments, the B cell engager is chosen from one or more of a CD40L, an OX40L, or a CD70 ligand, or an antibody molecule that binds to OX40, CD40 or CD70.

In other embodiments, the macrophage cell engager is chosen from one or more of a CD2 agonist; a CD40L; an OX40L; an antibody molecule that binds to OX40, CD40 or CD70; a Toll-like receptor agonist or a fragment thereof (e.g., a TLR4, e.g., a constitutively active TLR4 (caTLR4)); a CD47 agonist; or a STING agonist.

In other embodiments, the dendritic cell engager is chosen from one or more of a CD2 agonist, an OX40 antibody, an OX40L, 41BB agonist, a Toll-like receptor agonist or a fragment thereof (e.g., a TLR4, e.g., a constitutively active TLR4 (caTLR4)), CD47 agonist, or a STING agonist.

In one embodiment, the OX40L comprises the amino acid sequence:

(SEQ ID NO: 6210)
QVSHRYPRIQSIKVQFTEYKKEKGFILTSQKEDEIMKVQNNSVIINCDG
FYLISLKGYFSQEVNISLHYQKDEEPLFQLKKVRSVNSLMVASLTYKDK
VYLNVTTDNTSLDDFHVNGGELILIHQNPGEFCVL,

a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 6210.

In another embodiment the CD40L comprises the amino acid sequence:

(SEQ ID NO: 6211)
MQKGDQNPQIAAHVISEASSKTTSVLQWAEKGYYTMSNNLVTLENGKQL
TVKRQGLYYIYAQVTFCSNREASSQAPFIASLCLKSPGRFERILLRAAN
THSSAKPCGQQSIHLGGVFELQPGASVFVNVTDPSQVSHGTGFTSFGLL
KL,

a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 6211.

In yet other embodiments, the STING agonist comprises a cyclic dinucleotide, e.g., a cyclic di-GMP (cdGMP), a cyclic di-AMP (cdAMP), or a combination thereof, optionally with 2′,5′ or 3′,5′ phosphate linkages.

In one embodiment, the immune cell engager includes 41BB ligand, e.g., comprising the amino acid sequence:

(SEQ ID NO: 6212)
ACPWAVSGARASPGSAASPRLREGPELSPDDPAGLLDLRQGMFAQLVAQ
NVLLIDGPLSWYSDPGLAGVSLTGGLSYKEDTKELVVAKAGVYYVFFQL
ELRRVVAGEGSGSVSLALHLQPLRSAAGAAALALTVDLPPASSEARNSA
FGFQGRLLHLSAGQRLGVHLHTEARARHAWQLTQGATVLGLFRVTPEIP
AGLPSPRSE,

a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 6212.

### Toll-Like Receptors

Toll-Like Receptors (TLRs) are evolutionarily conserved receptors are homologues of the Drosophila Toll protein, and recognize highly conserved structural motifs known as pathogen-associated microbial patterns (PAMPs), which are exclusively expressed by microbial pathogens, or danger-associated molecular patterns (DAMPs) that are endogenous molecules released from necrotic or dying cells. PAMPs include various bacterial cell wall components such as lipopolysaccharide (LPS), peptidoglycan (PGN) and lipopeptides, as well as flagellin, bacterial DNA and viral double-stranded RNA. DAMPs include intracellular proteins such as heat shock proteins as well as protein fragments from the extracellular matrix. Stimulation of TLRs by the corresponding PAMPs or DAMPs initiates signaling cascades leading to the activation of transcription factors, such as AP-1, NF-κB and interferon regulatory factors (IRFs). Signaling by TLRs results in a variety of cellular responses, including the production of interferons (IFNs), pro-inflammatory cytokines and effector cytokines that direct the adaptive immune response. TLRs are implicated in a number of inflammatory and immune disorders and play a role in cancer (Rakoff-Nahoum S. & Medzhitov R., 2009. Toll-like receptors and cancer. Nat Revs Cancer 9:57-63.)

TLRs are type I transmembrane proteins characterized by an extracellular domain containing leucine-rich repeats (LRRs) and a cytoplasmic tail that contains a conserved region called the Toll/IL-1 receptor (TIR) domain. Ten human and twelve murine TLRs have been characterized, TLR1 to TLR10 in humans, and TLR1 to TLR9, TLR11, TLR12 and TLR13 in mice, the homolog of TLR10 being a pseudogene. TLR2 is essential for the recognition of a variety of PAMPs from Gram-positive bacteria, including bacterial lipoproteins, lipomannans and lipoteichoic acids. TLR3 is implicated in virus-derived double-stranded RNA. TLR4 is predominantly activated by lipopolysaccharide. TLR5 detects bacterial flagellin and TLR9 is required for response to unmethylated CpG DNA. Finally, TLR7 and TLR8 recognize small synthetic antiviral molecules, and single-stranded RNA was reported to be their natural ligand. TLR11 has been reported to recognize uropathogenic E. coli and a profilin-like protein from Toxoplasma gondii. The repertoire of specificities of the TLRs is apparently extended by the ability of TLRs to heterodimerize with one another. For example, dimers of TLR2 and TLR6 are required for responses to diacylated lipoproteins while TLR2 and TLR1 interact to recognize triacylated lipoproteins. Specificities of the TLRs are also influenced by various adapter and accessory molecules, such as MD-2 and CD14 that form a complex with TLR4 in response to LPS.

TLR signaling consists of at least two distinct pathways: a MyD88-dependent pathway that leads to the production of inflammatory cytokines, and a MyD88-independent pathway associated with the stimulation of IFN-β and the maturation of dendritic cells. The MyD88-dependent pathway is common to all TLRs, except TLR3 (Adachi O. et al., 1998. Targeted disruption of the MyD88 gene results in loss of IL-1- and IL-18-mediated function. Immunity. 9(1):143-50). Upon activation by PAMPs or DAMPs, TLRs hetero- or homodimerize inducing the recruitment of adaptor proteins via the cytoplasmic TIR domain. Individual TLRs induce different signaling responses by usage of the different adaptor molecules. TLR4 and TLR2 signaling requires the adaptor TIRAP/Mal, which is involved in the MyD88-dependent pathway. TLR3 triggers the production of IFN-β in response to double-stranded RNA, in a MyD88-independent manner, through the adaptor TRIF/TICAM-1. TRAM/TICAM-2 is another adaptor molecule involved in the MyD88-independent pathway which function is restricted to the TLR4 pathway.

TLR3, TLR7, TLR8 and TLR9 recognize viral nucleic acids and induce type I IFNs. The signaling mechanisms leading to the induction of type I IFNs differ depending on the TLR activated. They involve the interferon regulatory factors, IRFs, a family of transcription factors known to play a critical role in antiviral defense, cell growth and immune regulation. Three IRFs (IRF3, IRF5 and IRF7) function as direct transducers of virus-mediated TLR signaling. TLR3 and TLR4 activate IRF3 and IRF7, while TLR7 and TLR8 activate IRF5 and IRF7 (Doyle S. et al., 2002. IRF3 mediates a TLR3/TLR4-specific antiviral gene program. Immunity. 17(3):251-63). Furthermore, type I IFN production stimulated by TLR9 ligand CpG-A has been shown to be mediated by PI(3)K and mTOR (Costa-Mattioli M. & Sonenberg N. 2008. RAPping production of type I interferon in pDCs through mTOR. Nature Immunol. 9: 1097-1099).

TLR-9

TLR9 recognizes unmethylated CpG sequences in DNA molecules. CpG sites are relatively rare (˜1%) on vertebrate genomes in comparison to bacterial genomes or viral DNA. TLR9 is expressed by numerous cells of the immune system such as B lymphocytes, monocytes, natural killer (NK) cells, and plasmacytoid dendritic cells. TLR9 is expressed intracellularly, within the endosomal compartments and functions to alert the immune system of viral and bacterial infections by binding to DNA rich in CpG motifs. TLR9 signals leads to activation of the cells initiating pro-inflammatory reactions that result in the production of cytokines such as type-I interferon and IL-12.

TLR Agonists

A TLR agonist can agonize one or more TLR, e.g., one or more of human TLR-1, 2, 3, 4, 5, 6, 7, 8, 9, or 10. In some embodiments, an adjunctive agent described herein is a TLR agonist. In some embodiments, the TLR agonist specifically agonizes human TLR-9. In some embodiments, the TLR-9 agonist is a CpG moiety. As used herein, a CpG moiety, is a linear dinucleotide having the sequence: 5′-C-phosphate-G-3′, that is, cytosine and guanine separated by only one phosphate.

In some embodiments, the CpG moiety comprises at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more CpG dinucleotides. In some embodiments, the CpG moiety consists of 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 CpG dinucleotides. In some embodiments, the CpG moiety has 1-5, 1-10, 1-20, 1-30, 1-40, 1-50, 5-10, 5-20, 5-30, 10-20, 10-30, 10-40, or 10-50 CpG dinucleotides.

In some embodiments, the TLR-9 agonist is a synthetic ODN (oligodeoxynucleotides). CpG ODNs are short synthetic single-stranded DNA molecules containing unmethylated CpG dinucleotides in particular sequence contexts (CpG motifs). CpG ODNs possess a partially or completely phosphorothioated (PS) backbone, as opposed to the natural phosphodiester (PO) backbone found in genomic bacterial DNA. There are three major classes of CpG ODNs: classes A, B and C, which differ in their immunostimulatory activities. CpG-A ODNs are characterized by a PO central CpG-containing palindromic motif and a PS-modified 3′ poly-G string. They induce high IFN-α production from pDCs but are weak stimulators of TLR9-dependent NF-κB signaling and pro-inflammatory cytokine (e.g. IL-6) production. CpG-B ODNs contain a full PS backbone with one or more CpG dinucleotides. They strongly activate B cells and TLR9-dependent NF-κB signaling but weakly stimulate IFN-α secretion. CpG-C ODNs combine features of both classes A and B. They contain a complete PS backbone and a CpG-containing palindromic motif C-Class CpG ODNs induce strong IFN-α production from pDC as well as B cell stimulation.

### Cytokine Molecules

Cytokines are generally polypeptides that influence cellular activity, for example, through signal transduction pathways. Accordingly, a cytokine of the multispecific or multifunctional polypeptide is useful and can be associated with receptor-mediated signaling that transmits a signal from outside the cell membrane to modulate a response within the cell. Cytokines are proteinaceous signaling compounds that are mediators of the immune response. They control many different cellular functions including proliferation, differentiation and cell survival/apoptosis; cytokines are also involved in several pathophysiological processes including viral infections and autoimmune diseases. Cytokines are synthesized under various stimuli by a variety of cells of both the innate (monocytes, macrophages, dendritic cells) and adaptive (T- and B-cells) immune systems. Cytokines can be classified into two groups: pro- and anti-inflammatory. Pro-inflammatory cytokines, including IFNγ, IL-1, IL-6 and TNF-alpha, are predominantly derived from the innate immune cells and Th1 cells. Anti-inflammatory cytokines, including IL-10, IL-4, IL-13 and IL-5, are synthesized from Th2 immune cells.

The present disclosure provides, inter alia, multispecific (e.g., bi-, tri-, quad-specific) or multifunctional molecules, that include, e.g., are engineered to contain, one or more cytokine molecules, e.g., immunomodulatory (e.g., proinflammatory) cytokines and variants, e.g., functional variants, thereof. Accordingly, in some embodiments, the cytokine molecule is an interleukin or a variant, e.g., a functional variant thereof. In some embodiments the interleukin is a proinflammatory interleukin. In some embodiments the interleukin is chosen from interleukin-2 (IL-2), interleukin-12 (IL-12), interleukin-15 (IL-15), interleukin-18 (IL-18), interleukin-21 (IL-21), interleukin-7 (IL-7), or interferon gamma. In some embodiments, the cytokine molecule is a proinflammatory cytokine.

In certain embodiments, the cytokine is a single chain cytokine. In certain embodiments, the cytokine is a multichain cytokine (e.g., the cytokine comprises 2 or more (e.g., 2) polypeptide chains. An exemplary multichain cytokine is IL-12.

Examples of useful cytokines include, but are not limited to, GM-CSF, IL-1α, IL-1β, IL-2, IL-3, IL-4, IL-5, IL-6, IL-7, IL-8, IL-10, IL-12, IL-21, IFN-α, IFN-β, IFN-γ, MIP-1α, MIP-1β, TGF-β, TNF-α, and TNFβ. In one embodiment the cytokine of the multispecific or multifunctional polypeptide is a cytokine selected from the group of GM-CSF, IL-2, IL-7, IL-8, IL-10, IL-12, IL-15, IL-21, IFN-α, IFN-γ, MIP-1α, MIP-1p and TGF-β. In one embodiment the cytokine of the multispecific or multifunctional polypeptide is a cytokine selected from the group of IL-2, IL-7, IL-10, IL-12, IL-15, IFN-α, and IFN-γ. In certain embodiments the cytokine is mutated to remove N- and/or O-glycosylation sites. Elimination of glycosylation increases homogeneity of the product obtainable in recombinant production. In certain embodiments, the cytokine is TGF-β. In certain embodiments, the multispecific or multifunctional polypeptide comprises a TGF-β inhibitor.

In one embodiment, the cytokine of the multispecific or multifunctional polypeptide is IL-2. In a specific embodiment, the IL-2 cytokine can elicit one or more of the cellular responses selected from the group consisting of: proliferation in an activated T lymphocyte cell, differentiation in an activated T lymphocyte cell, cytotoxic T cell (CTL) activity, proliferation in an activated B cell, differentiation in an activated B cell, proliferation in a natural killer (NK) cell, differentiation in a NK cell, cytokine secretion by an activated T cell or an NK cell, and NK/lymphocyte activated killer (LAK) antitumor cytotoxicity. In another particular embodiment the IL-2 cytokine is a mutant IL-2 cytokine having reduced binding affinity to the .alpha.subunit of the IL-2 receptor. Together with the .beta. and .gamma.subunits (also known as CD122 and CD132, respectively), the .alpha.subunit (also known as CD25) forms the heterotrimeric high-affinity IL-2 receptor, while the dimeric receptor consisting only of the β- and γ-subunits is termed the intermediate-affinity IL-2 receptor. As described in PCT patent application number PCT/EP2012/051991, which is incorporated herein by reference in its entirety, a mutant IL-2 polypeptide with reduced binding to the .alpha.subunit of the IL-2 receptor has a reduced ability to induce IL-2 signaling in regulatory T cells, induces less activation-induced cell death (AICD) in T cells, and has a reduced toxicity profile in vivo, compared to a wild-type IL-2 polypeptide. The use of such an cytokine with reduced toxicity is particularly advantageous in a multispecific or multifunctional polypeptide according to the invention, having a long serum half-life due to the presence of an Fc domain. In one embodiment, the mutant IL-2 cytokine of the multispecific or multifunctional polypeptide according to the invention comprises at least one amino acid mutation that reduces or abolishes the affinity of the mutant IL-2 cytokine to the .alpha.subunit of the IL-2 receptor (CD25) but preserves the affinity of the mutant IL-2 cytokine to the intermediate-affinity IL-2 receptor (consisting of the β and γ subunits of the IL-2 receptor), compared to the non-mutated IL-2 cytokine. In one embodiment the one or more amino acid mutations are amino acid substitutions. In a specific embodiment, the mutant IL-2 cytokine comprises one, two or three amino acid substitutions at one, two or three position(s) selected from the positions corresponding to residue 42, 45, and 72 of human IL-2. In a more specific embodiment, the mutant IL-2 cytokine comprises three amino acid substitutions at the positions corresponding to residue 42, 45 and 72 of human IL-2. In an even more specific embodiment, the mutant IL-2 cytokine is human IL-2 comprising the amino acid substitutions F42A, Y45A and L72G. In one embodiment the mutant IL-2 cytokine additionally comprises an amino acid mutation at a position corresponding to position 3 of human IL-2, which eliminates the O-glycosylation site of IL-2. Particularly, said additional amino acid mutation is an amino acid substitution replacing a threonine residue by an alanine residue. A particular mutant IL-2 cytokine useful in the invention comprises four amino acid substitutions at positions corresponding to residues 3, 42, 45 and 72 of human IL-2. Specific amino acid substitutions are T3A, F42A, Y45A and L72G. As demonstrated in PCT patent application number PCT/EP2012/051991 and in the appended Examples, said quadruple mutant IL-2 polypeptide (IL-2 qm) exhibits no detectable binding to CD25, reduced ability to induce apoptosis in T cells, reduced ability to induce IL-2 signaling in T.sub.reg cells, and a reduced toxicity profile in vivo. However, it retains ability to activate IL-2 signaling in effector cells, to induce proliferation of effector cells, and to generate IFN-γ as a secondary cytokine by NK cells.

The IL-2 or mutant IL-2 cytokine according to any of the above embodiments may comprise additional mutations that provide further advantages such as increased expression or stability. For example, the cysteine at position 125 may be replaced with a neutral amino acid such as alanine, to avoid the formation of disulfide-bridged IL-2 dimers. Thus, in certain embodiments the IL-2 or mutant IL-2 cytokine of the multispecific or multifunctional polypeptide according to the invention comprises an additional amino acid mutation at a position corresponding to residue 125 of human IL-2. In one embodiment said additional amino acid mutation is the amino acid substitution C125A.

In a specific embodiment, the IL-2 cytokine of the multispecific or multifunctional polypeptide comprises the polypeptide sequence of SEQ ID NO: 6364

[APTSSSTKKTQLQLEHLLLDLQMILNGINNYKNPKLTRMLTFKFYMPK
KATELKHLQCLEEELKPLEEVLNLAQSKNFHLRPRDLISNINVIVLELK
GSETTFMCEYADETATIVEFLNRWITFAQSIISTLT].

In another specific embodiment the IL-2 cytokine of the multispecific or multifunctional polypeptide comprises the polypeptide sequence of SEQ ID NO: 6365

[APASSSTKKTQLQLEHLLLDLQMILNGINNYKNPKLTRMLTAKFAMPK
KATELKHLQCLEEELKPLEEVLNGAQSKNFHLRPRDLISNINVIVLELK
GSETTFMCEYADETATIVEFLNRWITFAQSIISTLT].

In another embodiment, the cytokine of the multispecific or multifunctional polypeptide is IL-12. In a specific embodiment, said IL-12 cytokine is a single chain IL-12 cytokine. In an even more specific embodiment, the single chain IL-12 cytokine comprises the polypeptide sequence of SEQ ID NO: 6366

[IWELKKDVYVVELDWYPDAPGEMVVLTCDTPEEDGITWTLDQSSEVLG
SGKTLTIQVKEFGDAGQYTCHKGGEVLSHSLLLLHKKEDGIWSTDILKD
QKEPKNKTFLRCEAKNYSGRFTCWWLTTISTDLTFSVKSSRGSSDPQGV
TCGAATLSAERVRGDNKEYEYSVECQEDSACPAAEESLPIEVMVDAVHK
LKYENYTSSFFIRDIIKPDPPKNLQLKPLKNSRQVEVSWEYPDTWSTPH
SYFSLTFCVQVQGKSKREKKDRVFTDKTSATVICRKNASISVRAQDRYY
SSSWSEWASVPCSGGGGSGGGGSGGGGSRNLPVATPDPGMFPCLHHSQN
LLRAVSNMLQKARQTLEFYPCTSEEIDHEDITKDKTSTVEACLPLELTK
NESCLNSRETSFITNGSCLASRKTSFMMALCLSSIYEDLKMYQVEFKTM
NAKLLMDPKRQIFLDQNMLAVIDELMQALNFNSETVPQKSSLEEPDFYK
TKIKLCILLHAFRIRAVTIDRVMSYLNAS].

In one embodiment, the IL-12 cytokine can elicit one or more of the cellular responses selected from the group consisting of: proliferation in a NK cell, differentiation in a NK cell, proliferation in a T cell, and differentiation in a T cell.

In another embodiment, the cytokine of the multispecific or multifunctional polypeptide is IL-10. In a specific embodiment, said IL-10 cytokine is a single chain IL-10 cytokine. In an even more specific embodiment, the single chain IL-10 cytokine comprises the polypeptide sequence of SEQ ID NO: 6367

[SPGQGTQSENSCTHFPGNLPNMLRDLRDAFSRVKTFFQMKDQLDNLLL
KESLLEDFKGYLGCQALSEMIQFYLEEVMPQAENQDPDIKAHVNSLGEN
LKTLRLRLRRCHRFLPCENKSKAVEQVKNAFNKLQEKGIYKAMSEFDIF
INYIEAYMTMKIRNGGGGSGGGGSGGGGSGGGGSSPGQGTQSENSCTHF
PGNLPNMLRDLRDAFSRVKTFFQMKDQLDNLLLKESLLEDFKGYLGCQA
LSEMIQFYLEEVMPQAENQDPDIKAHVNSLGENLKTLRLRLRRCHRFLP
CENKSKAVEQVKNAFNKLQEKGIYKAMSEFDIFINYIEAYMTMKIRN].

In another specific embodiment, the IL-10 cytokine is a monomeric IL-10 cytokine. In a more specific embodiment, the monomeric IL-10 cytokine comprises the polypeptide sequence of SEQ ID NO: 6368

[SPGQGTQSENSCTHFPGNLPNMLRDLRDAFSRVKTFFQMKDQLDNLLL
KESLLEDFKGYLGCQALSEMIQFYLEEVMPQAENQDPDIKAHVNSLGEN
LKTLRLRLRRCHRFLPCENGGGSGGKSKAVEQVKNAFNKLQEKGIYKAM
SEFDIFINYIEAYMTMKIRN].

In one embodiment, the IL-10 cytokine can elicit one or more of the cellular responses selected from the group consisting of: inhibition of cytokine secretion, inhibition of antigen presentation by antigen presenting cells, reduction of oxygen radical release, and inhibition of T cell proliferation. A multispecific or multifunctional polypeptide according to the invention wherein the cytokine is IL-10 is particularly useful for downregulation of inflammation, e.g. in the treatment of an inflammatory disorder.

In another embodiment, the cytokine of the multispecific or multifunctional polypeptide is IL-15. In a specific embodiment said IL-15 cytokine is a mutant IL-15 cytokine having reduced binding affinity to the α-subunit of the IL-15 receptor. Without wishing to be bound by theory, a mutant IL-15 polypeptide with reduced binding to the .alpha.subunit of the IL-15 receptor has a reduced ability to bind to fibroblasts throughout the body, resulting in improved pharmacokinetics and toxicity profile, compared to a wild-type IL-15 polypeptide. The use of an cytokine with reduced toxicity, such as the described mutant IL-2 and mutant IL-15 effector moieties, is particularly advantageous in a multispecific or multifunctional polypeptide according to the invention, having a long serum half-life due to the presence of an Fc domain. In one embodiment the mutant IL-15 cytokine of the multispecific or multifunctional polypeptide according to the invention comprises at least one amino acid mutation that reduces or abolishes the affinity of the mutant IL-15 cytokine to the .alpha.subunit of the IL-15 receptor but preserves the affinity of the mutant IL-15 cytokine to the intermediate-affinity IL-15/IL-2 receptor (consisting of the .beta. and .gamma.subunits of the IL-15/IL-2 receptor), compared to the non-mutated IL-15 cytokine. In one embodiment the amino acid mutation is an amino acid substitution. In a specific embodiment, the mutant IL-15 cytokine comprises an amino acid substitution at the position corresponding to residue 53 of human IL-15. In a more specific embodiment, the mutant IL-15 cytokine is human IL-15 comprising the amino acid substitution E53A. In one embodiment the mutant IL-15 cytokine additionally comprises an amino acid mutation at a position corresponding to position 79 of human IL-15, which eliminates the N-glycosylation site of IL-15. Particularly, said additional amino acid mutation is an amino acid substitution replacing an asparagine residue by an alanine residue. In an even more specific embodiment the IL-15 cytokine comprises the polypeptide sequence of SEQ ID NO: 6370 [NWVNVISDLKKIEDLIQSMHIDATLYTESDVHPSCKVTAMKCFLLELQVISLASGDASIHDTVEN LIILANNSLSSNGAVTESGCKECEELEEKNIKEFLQSFVHIVQMFINTS]. In one embodiment, the IL-15 cytokine can elicit one or more of the cellular responses selected from the group consisting of: proliferation in an activated T lymphocyte cell, differentiation in an activated T lymphocyte cell, cytotoxic T cell (CTL) activity, proliferation in an activated B cell, differentiation in an activated B cell, proliferation in a natural killer (NK) cell, differentiation in a NK cell, cytokine secretion by an activated T cell or an NK cell, and NK/lymphocyte activated killer (LAK) antitumor cytotoxicity.

Mutant cytokine molecules useful as effector moieties in the multispecific or multifunctional polypeptide can be prepared by deletion, substitution, insertion or modification using genetic or chemical methods well known in the art. Genetic methods may include site-specific mutagenesis of the encoding DNA sequence, PCR, gene synthesis, and the like. The correct nucleotide changes can be verified for example by sequencing. Substitution or insertion may involve natural as well as non-natural amino acid residues. Amino acid modification includes well known methods of chemical modification such as the addition or removal of glycosylation sites or carbohydrate attachments, and the like.

In one embodiment, the cytokine, particularly a single-chain cytokine, of the multispecific or multifunctional polypeptide is GM-CSF. In a specific embodiment, the GM-CSF cytokine can elicit proliferation and/or differentiation in a granulocyte, a monocyte or a dendritic cell. In one embodiment, the cytokine, particularly a single-chain cytokine, of the multispecific or multifunctional polypeptide is IFN-α. In a specific embodiment, the IFN-α cytokine can elicit one or more of the cellular responses selected from the group consisting of: inhibiting viral replication in a virus-infected cell, and upregulating the expression of major histocompatibility complex I (MHC I). In another specific embodiment, the IFN-α cytokine can inhibit proliferation in a tumor cell. In one embodiment the cytokine, particularly a single-chain cytokine, of the multispecific or multifunctional polypeptide is IFNγ. In a specific embodiment, the IFN-γ cytokine can elicit one or more of the cellular responses selected from the group of: increased macrophage activity, increased expression of MHC molecules, and increased NK cell activity. In one embodiment the cytokine, particularly a single-chain cytokine, of the multispecific or multifunctional polypeptide is IL-7. In a specific embodiment, the IL-7 cytokine can elicit proliferation of T and/or B lymphocytes. In one embodiment, the cytokine, particularly a single-chain cytokine, of the multispecific or multifunctional polypeptide is IL-8. In a specific embodiment, the IL-8 cytokine can elicit chemotaxis in neutrophils. In one embodiment, the cytokine, particularly a single-chain cytokine, of the multispecific or multifunctional polypeptide, is MIP-1α. In a specific embodiment, the MIP-1α cytokine can elicit chemotaxis in monocytes and T lymphocyte cells. In one embodiment, the cytokine, particularly a single-chain cytokine, of the multispecific or multifunctional polypeptide is MIP-1β. In a specific embodiment, the MIP-1β cytokine can elicit chemotaxis in monocytes and T lymphocyte cells. In one embodiment, the cytokine, particularly a single-chain cytokine, of the multispecific or multifunctional polypeptide is TGF-β. In a specific embodiment, the TGF-β cytokine can elicit one or more of the cellular responses selected from the group consisting of: chemotaxis in monocytes, chemotaxis in macrophages, upregulation of IL-1 expression in activated macrophages, and upregulation of IgA expression in activated B cells.

In one embodiment, the multispecific or multifunctional polypeptide of the invention binds to an cytokine receptor with a dissociation constant (K_{D}) that is at least about 1, 1.5, 2, 2.5, 3, 3.5, 4, 4.5, 5, 5.5, 6, 6.5, 7, 7.5, 8, 8.5, 9, 9.5 or 10 times greater than that for a control cytokine. In another embodiment, the multispecific or multifunctional polypeptide binds to an cytokine receptor with a K_{D} that is at least 2, 3, 4, 5, 6, 7, 8, 9, or 10 times greater than that for a corresponding multispecific or multifunctional polypeptide comprising two or more effector moieties. In another embodiment, the multispecific or multifunctional polypeptide binds to an cytokine receptor with a dissociation constant K_{D} that is about 10 times greater than that for a corresponding the multispecific or multifunctional polypeptide comprising two or more cytokines.

In some embodiments, the multispecific molecules disclosed herein include a cytokine molecule. In some embodiments, the cytokine molecule includes a full length, a fragment or a variant of a cytokine; a cytokine receptor domain, e.g., a cytokine receptor dimerizing domain; or an agonist of a cytokine receptor, e.g., an antibody molecule (e.g., an agonistic antibody) to a cytokine receptor.

In some embodiments the cytokine molecule is chosen from IL-2, IL-12, IL-15, IL-18, IL-7, IL-21, or interferon gamma, or a fragment or variant thereof, or a combination of any of the aforesaid cytokines. The cytokine molecule can be a monomer or a dimer. In some embodiments, the cytokine molecule can further include a cytokine receptor dimerizing domain.

In other embodiments, the cytokine molecule is an agonist of a cytokine receptor, e.g., an antibody molecule (e.g., an agonistic antibody) to a cytokine receptor chosen from an IL-15Ra or IL-21R.

In one embodiment, the cytokine molecule is IL-15, e.g., human IL-15 (e.g., comprising the amino acid sequence.

(SEQ ID NO: 6191)
NWVNVISDLKKIEDLIQSMHIDATLYTESDVHPSCKVTAMKCFLLELQV
ISLESGDASIHDTVENLIILANNSLSSNGNVTESGCKECEELEEKNIKE
FLQSFVHIVQMFINTS,

a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 6191.

In some embodiments, the cytokine molecule comprises a receptor dimerizing domain, e.g., an IL15Ralpha dimerizing domain. In one embodiment, the IL15Ralpha dimerizing domain comprises the amino acid sequence:

(SEQ ID NO: 6192)
MAPRRARGCRTLGLPALLLLLLLRPPATRGITCPPPMSVEHADIWVKSY
SLYSRERYICNSGFKRKAGTSSLTECVL,

a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 6192. In some embodiments, the cytokine molecule (e.g., IL-15) and the receptor dimerizing domain (e.g., an IL15Ralpha dimerizing domain) of the multispecific molecule are covalently linked, e.g., via a linker (e.g., a Gly-Ser linker, e.g., a linker comprising the amino acid sequence SGGSGGGGSGGGSGGGGSLQ (SEQ ID NO: 6193). In other embodiments, the cytokine molecule (e.g., IL-15) and the receptor dimerizing domain (e.g., an IL15Ralpha dimerizing domain) of the multispecific molecule are not covalently linked, e.g., are non-covalently associated.

In other embodiments, the cytokine molecule is IL-2, e.g., human IL-2 (e.g., comprising the amino acid sequence:

(SEQ ID NO: 6194)
APTSSSTKKTQLQLEHLLLDLQMILNGINNYKNPKLTRMLTFKFYMPKK
ATELKHLQCLEEELKPLEEVLNLAQSKNFHLRPRDLISNINVIVLELKG
SETTFMCEYADETATIVEFLNRWITFCQSIISTLT,

a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO:6194).

In other embodiments, the cytokine molecule is IL-18, e.g., human IL-18 (e.g., comprising the amino acid sequence:

(SEQ ID NO: 6195)
YFGKLESKLSVIRNLNDQVLFIDQGNRPLFEDMTDSDCRDNAPRTIFII
SMYKDSQPRGMAVTISVKCEKISTLSCENKIISFKEMNPPDNIKDTKSD
IIFFQRSVPGHDNKMQFESSSYEGYFLACEKERDLFKLILKKEDELGDR
SIMFTVQNED,

a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 6195).

In other embodiments, the cytokine molecule is IL-21, e.g., human IL-21 (e.g., comprising the amino acid sequence:

(SEQ ID NO: 6196)
QGQDRHMIRMRQLIDIVDQLKNYVNDLVPEFLPAPEDVETNCEWSAFSC
FQKAQLKSANTGNNERIINVSIKKLKRKPPSTNAGRRQKHRLTCPSCDS
YEKKPPKEFLERFKSLLQKMIHQHLSSRTHGSEDS,

a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 6196).

In yet other embodiments, the cytokine molecule is interferon gamma, e.g., human interferon gamma (e.g., comprising the amino acid sequence:

(SEQ ID NO: 6197)
QDPYVKEAENLKKYFNAGHSDVADNGTLFLGILKNWKEESDRKIMQSQI
VSFYFKLFKNFKDDQSIQKSVETIKEDMNVKFFNSNKKKRDDFEKLTNY
SVTDLNVQRKAIHELIQVMAELSPAAKTGKRKRSQMLFRG,

a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 6197).

### TGF-β Inhibitor

In one aspect, provided herein is a multispecific or multifunctional polypeptide (e.g., antibody molecule) comprising a modulator of TGF-β (e.g., a TGF-β inhibitor). In some embodiments, the TGF-β inhibitor binds to and inhibits TGF-β, e.g., reduces the activity of TGF-β. In some embodiments, the TGF-β inhibitor inhibits (e.g., reduces the activity of) TGF-β 1. In some embodiments, the TGF-β inhibitor inhibits (e.g., reduces the activity of) TGF-β 2. In some embodiments, the TGF-β inhibitor inhibits (e.g., reduces the activity of) TGF-β 3. In some embodiments, the TGF-β inhibitor inhibits (e.g., reduces the activity of) TGF-β 1 and TGF-β 3. In some embodiments, the TGF-β inhibitor inhibits (e.g., reduces the activity of) TGF-β 1, TGF-β 2, and TGF-β 3.

In some embodiments, the TGF-β inhibitor comprises a portion of a TGF-β receptor (e.g., an extracellular domain of a TGF-β receptor) that is capable of inhibiting (e.g., reducing the activity of) TGF-β, or functional fragment or variant thereof. In some embodiments, the TGF-β inhibitor comprises a TGFBR1 polypeptide (e.g., an extracellular domain of TGFBR1 or functional variant thereof). In some embodiments, the TGF-β inhibitor comprises a TGFBR2 polypeptide (e.g., an extracellular domain of TGFBR2 or functional variant thereof). In some embodiments, the TGF-β inhibitor comprises a TGFBR3 polypeptide (e.g., an extracellular domain of TGFBR3 or functional variant thereof). In some embodiments, the TGF-β inhibitor comprises a TGFBR1 polypeptide (e.g., an extracellular domain of TGFBR1 or functional variant thereof) and a TGFBR2 polypeptide (e.g., an extracellular domain of TGFBR2 or functional variant thereof). In some embodiments, the TGF-β inhibitor comprises a TGFBR1 polypeptide (e.g., an extracellular domain of TGFBR1 or functional variant thereof) and a TGFBR3 polypeptide (e.g., an extracellular domain of TGFBR3 or functional variant thereof). In some embodiments, the TGF-β inhibitor comprises a TGFBR2 polypeptide (e.g., an extracellular domain of TGFBR2 or functional variant thereof) and a TGFBR3 polypeptide (e.g., an extracellular domain of TGFBR3 or functional variant thereof).

Exemplary TGF-β receptor polypeptides that can be used as TGF-β inhibitors have been disclosed in U.S. Pat. Nos. 8,993,524, 9,676,863, 8,658,135, US20150056199, US20070184052, and WO2017037634, all of which are herein incorporated by reference in their entirety.

In some embodiments, the TGF-β inhibitor comprises an extracellular domain of TGFBR1 or a sequence substantially identical thereto (e.g., a sequence that is at least 80%, 85%, 90%, or 95% identical thereto). In some embodiments, the TGF-β inhibitor comprises an extracellular domain of SEQ ID NO: 6381, or a sequence substantially identical thereto (e.g., a sequence that is at least 80%, 85%, 90%, or 95% identical thereto). In some embodiments, the TGF-β inhibitor comprises an extracellular domain of SEQ ID NO: 6382, or a sequence substantially identical thereto (e.g., a sequence that is at least 80%, 85%, 90%, or 95% identical thereto). In some embodiments, the TGF-β inhibitor comprises an extracellular domain of SEQ ID NO: 6383, or a sequence substantially identical thereto (e.g., a sequence that is at least 80%, 85%, 90%, or 95% identical thereto). In some embodiments, the TGF-β inhibitor comprises the amino acid sequence of SEQ ID NO: 6390, or a sequence substantially identical thereto (e.g., a sequence that is at least 80%, 85%, 90%, or 95% identical thereto). In some embodiments, the TGF-β inhibitor comprises the amino acid sequence of SEQ ID NO: 6391, or a sequence substantially identical thereto (e.g., a sequence that is at least 80%, 85%, 90%, or 95% identical thereto).

In some embodiments, the TGF-β inhibitor comprises an extracellular domain of TGFBR2 or a sequence substantially identical thereto (e.g., a sequence that is at least 80%, 85%, 90%, or 95% identical thereto). In some embodiments, the TGF-β inhibitor comprises an extracellular domain of SEQ ID NO: 6384, or a sequence substantially identical thereto (e.g., a sequence that is at least 80%, 85%, 90%, or 95% identical thereto). In some embodiments, the TGF-β inhibitor comprises an extracellular domain of SEQ ID NO: 6385, or a sequence substantially identical thereto (e.g., a sequence that is at least 80%, 85%, 90%, or 95% identical thereto). In some embodiments, the TGF-β inhibitor comprises the amino acid sequence of SEQ ID NO: 6386, or a sequence substantially identical thereto (e.g., a sequence that is at least 80%, 85%, 90%, or 95% identical thereto). In some embodiments, the TGF-β inhibitor comprises the amino acid sequence of SEQ ID NO: 6387, or a sequence substantially identical thereto (e.g., a sequence that is at least 80%, 85%, 90%, or 95% identical thereto). In some embodiments, the TGF-β inhibitor comprises the amino acid sequence of SEQ ID NO: 6388, or a sequence substantially identical thereto (e.g., a sequence that is at least 80%, 85%, 90%, or 95% identical thereto). In some embodiments, the TGF-β inhibitor comprises the amino acid sequence of SEQ ID NO: 6389, or a sequence substantially identical thereto (e.g., a sequence that is at least 80%, 85%, 90%, or 95% identical thereto).

In some embodiments, the TGF-β inhibitor comprises an extracellular domain of TGFBR3 or a sequence substantially identical thereto (e.g., a sequence that is at least 80%, 85%, 90%, or 95% identical thereto). In some embodiments, the TGF-β inhibitor comprises an extracellular domain of SEQ ID NO: 6392, or a sequence substantially identical thereto (e.g., a sequence that is at least 80%, 85%, 90%, or 95% identical thereto). In some embodiments, the TGF-β inhibitor comprises an extracellular domain of SEQ ID NO: 6393, or a sequence substantially identical thereto (e.g., a sequence that is at least 80%, 85%, 90%, or 95% identical thereto). In some embodiments, the TGF-β inhibitor comprises the amino acid sequence of SEQ ID NO: 6394, or a sequence substantially identical thereto (e.g., a sequence that is at least 80%, 85%, 90%, or 95% identical thereto).

In some embodiments, the TGF-β inhibitor comprises no more than one TGF-β receptor extracellular domain. In some embodiments, the TGF-β inhibitor comprises two or more (e.g., two, three, four, five, or more) TGF-β receptor extracellular domains, linked together, e.g., via a linker.

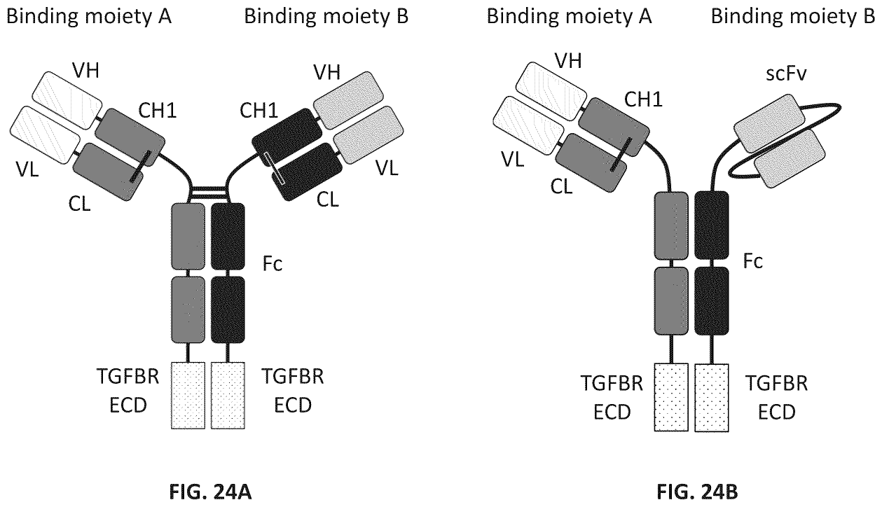

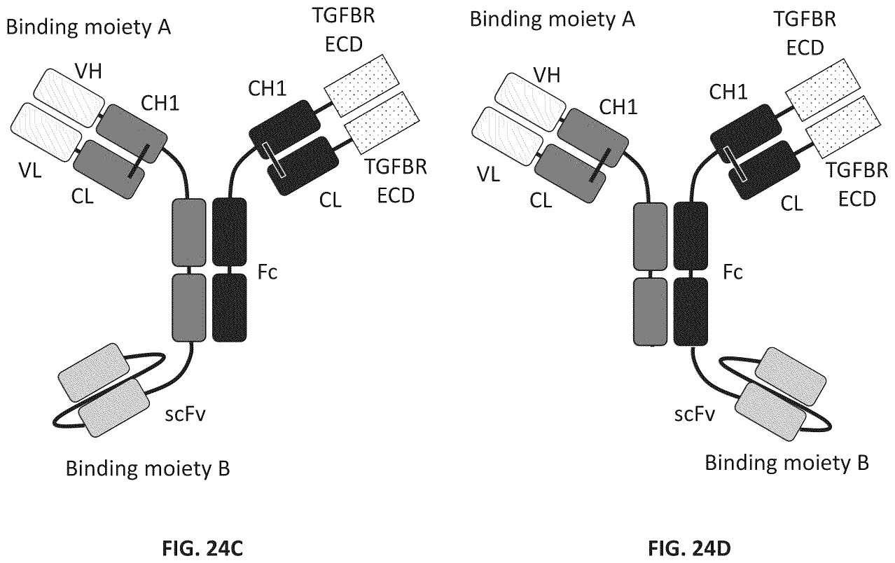

In some embodiments, the multispecific molecule comprises a configuration shown in FIGS. 24A-24D. In some embodiments, the TGFRβ inhibitor comprises a TGF-beta receptor ECD homodimer. In some embodiments, the TGFRβ inhibitor comprises a TGF-beta receptor ECD heterodimer. In some embodiments, the two TGFBR ECD domains are linked to two Fc regions, e.g., the C-terminus of two Fc regions. In some embodiments, the two TGFBR ECD domains are linked to CH1 and CL, respectively.

TABLE 37
Exemplary amino acid sequences of TGF-β polypeptides or TGF-β receptor polypeptides

| SEQ ID | | |
|---|---|---|
| NO | Description | Amino acid sequence |
| SEQ ID | Immature | MPPSGLRLLLLLLPLLWLLVLTPGRPAAGLSTCKTIDMELVKRKRIE |
| NO: | human | AIRGQILSKLRLASPPSQGEVPPGPLPEAVLALYNSTRDRVAGESAEP |
| 6378 | TGF-ß 1 | EPEPEADYYAKEVTRVLMVETHNEIYDKFKQSTHSIYMFFNTSELRE |
| | (P01137-1) | AVPEPVLLSRAELRLLRLKLKVEQHVELYQKYSNNSWRYLSNRLLA |
| | | PSDSPEWLSFDVTGVVRQWLSRGGEIEGFRLSAHCSCDSRDNTLQV |
| | | DINGFTTGRRGDLATIHGMNRPFLLLMATPLERAQHLQSSRHRRAL |
| | | DTNYCFSSTEKNCCVRQLYIDFRKDLGWKWIHEPKGYHANFCLGPC |
| | | PYIWSLDTQYSKVLALYNQHNPGASAAPCCVPQALEPLPIVYYVGR |
| | | KPKVEQLSNMIVRSCKCS |
| SEQ ID | Human | LSTCKTIDMELVKRKRIEAIRGQILSKLRLASPPSQGEVPPGPLPEAVL |
| NO: | TGF-ß 1 | ALYNSTRDRVAGESAEPEPEPEADYYAKEVTRVLMVETHNEIYDKF |
| 6395 | (P01137-1) | KQSTHSIYMFFNTSELREAVPEPVLLSRAELRLLRLKLKVEQHVELY |
| | | QKYSNNSWRYLSNRLLAPSDSPEWLSFDVTGVVRQWLSRGGEIEGF |
| | | RLSAHCSCDSRDNTLQVDINGFTTGRRGDLATIHGMNRPFLLLMAT |
| | | PLERAQHLQSSRHRRALDTNYCFSSTEKNCCVRQLYIDFRKDLGWK |
| | | WIHEPKGYHANFCLGPCPYIWSLDTQYSKVLALYNQHNPGASAAPC |
| | | CVPQALEPLPIVYYVGRKPKVEQLSNMIVRSCKCS |
| SEQ ID | Immature | MHYCVLSAFLILHLVTVALSLSTCSTLDMDQFMRKRIEAIRGQILSK |
| NO: | human | LKLTSPPEDYPEPEEVPPEVISIYNSTRDLLQEKASRRAAACERERSD |
| 6379 | TGF-ß 2 | EEYYAKEVYKIDMPPFFPSENAIPPTFYRPYFRIVRFDVSAMEKNASN |
| | (P61812-1) | LVKAEFRVFRLQNPKARVPEQRIELYQILKSKDLTSPTQRYIDSKVV |
| | | KTRAEGEWLSFDVTDAVHEWLHHKDRNLGFKISLHCPCCTFVPSNN |
| | | YIIPNKSEELEARFAGIDGTSTYTSGDQKTIKSTRKKNSGKTPHLLLM |
| | | LLPSYRLESQQTNRRKKRALDAAYCFRNVQDNCCLRPLYIDFKRDL |
| | | GWKWIHEPKGYNANFCAGACPYLWSSDTQHSRVLSLYNTINPEASA |
| | | SPCCVSQDLEPLTILYYIGKTPKIEQLSNMIVKSCKCS |
| SEQ ID | Human | LSTCSTLDMDQFMRKRIEAIRGQILSKLKLTSPPEDYPEPEEVPPEVIS |
| NO: | TGF-ß 2 | IYNSTRDLLQEKASRRAAACERERSDEEYYAKEVYKIDMPPFFPSEN |
| 6396 | (P61812-1) | AIPPTFYRPYFRIVRFDVSAMEKNASNLVKAEFRVFRLQNPKARVPE |
| | | QRIELYQILKSKDLTSPTQRYIDSKVVKTRAEGEWLSFDVTDAVHE |
| | | WLHHKDRNLGFKISLHCPCCTFVPSNNYIIPNKSEELEARFAGIDGTS |
| | | TYTSGDQKTIKSTRKKNSGKTPHLLLMLLPSYRLESQQTNRRKKRA |
| | | LDAAYCFRNVQDNCCLRPLYIDFKRDLGWKWIHEPKGYNANFCAG |
| | | ACPYLWSSDTQHSRVLSLYNTINPEASASPCCVSQDLEPLTILYYIGK |
| | | TPKIEQLSNMIVKSCKCS |
| SEQ ID | Immature | MKMHLQRALVVLALLNFATVSLSLSTCTTLDFGHIKKKRVEAIRGQI |
| NO: | human | LSKLRLTSPPEPTVMTHVPYQVLALYNSTRELLEEMHGEREEGCTQE |
| 6380 | TGF-β 3 | NTESEYYAKEIHKFDMIQGLAEHNELAVCPKGITSKVFRFNVSSVEK |
| | (P10600-1) | NRTNLFRAEFRVLRVPNPSSKRNEQRIELFQILRPDEHIAKQRYIGGK |
| | | NLPTRGTAEWLSFDVTDTVREWLLRRESNLGLEISIHCPCHTFQPNG |
| | | DILENIHEVMEIKFKGVDNEDDHGRGDLGRLKKQKDHHNPHLILM |
| | | MIPPHRLDNPGQGGQRKKRALDTNYCFRNLEENCCVRPLYIDFRQD |
| | | LGWKWVHEPKGYYANFCSGPCPYLRSADTTHSTVLGLYNTLNPEA |
| | | SASPCCVPQDLEPLTILYYVGRTPKVEQLSNMVVKSCKCS |
| SEQ ID | Human | LSTCTTLDFGHIKKKRVEAIRGQILSKLRLTSPPEPTVMTHVPYQVLA |
| NO: | TGF-ß 3 | LYNSTRELLEEMHGEREEGCTQENTESEYYAKEIHKFDMIQGLAEH |
| 6397 | (P10600-1) | NELAVCPKGITSKVFRFNVSSVEKNRTNLFRAEFRVLRVPNPSSKRN |
| | | EQRIELFQILRPDEHIAKQRYIGGKNLPTRGTAEWLSFDVTDTVREW |
| | | LLRRESNLGLEISIHCPCHTFQPNGDILENIHEVMEIKFKGVDNEDDH |
| | | GRGDLGRLKKQKDHHNPHLILMMIPPHRLDNPGQGGQRKKRALDT |
| | | NYCFRNLEENCCVRPLYIDFRQDLGWKWVHEPKGYYANFCSGPCP |
| | | YLRSADTTHSTVLGLYNTLNPEASASPCCVPQDLEPLTILYYVGRTP |
| | | KVEQLSNMVVKSCKCS |
| SEQ ID | Immature | MEAAVAAPRPRLLLLVLAAAAAAAAALLPGATALQCFCHLCTKDN |
| NO: | human | FTCVTDGLCFVSVTETTDKVIHNSMCIAEIDLIPRDRPFVCAPSSKTG |
| 6381 | TGFBR1 | SVTTTYCCNQDHCNKIELPTTVKSSPGLGPVELAAVIAGPVCFVCISL |
| | isoform 1 | MLMVYICHNRTVIHHRVPNEEDPSLDRPFISEGTTLKDLIYDMTTSG |
| | (P36897-1) | SGSGLPLLVQRTIARTIVLQESIGKGRFGEVWRGKWRGEEVAVKIFS |
| | | SREERSWFREAEIYQTVMLRHENILGFIAADNKDNGTWTQLWLVSD |
| | | YHEHGSLFDYLNRYTVTVEGMIKLALSTASGLAHLHMEIVGTQGKP |
| | | AIAHRDLKSKNILVKKNGTCCIADLGLAVRHDSATDTIDIAPNHRVG |
| | | TKRYMAPEVLDDSINMKHFESFKRADIYAMGLVFWEIARRCSIGGIH |
| | | EDYQLPYYDLVPSDPSVEEMRKVVCEQKLRPNIPNRWQSCEALRV |
| | | MAKIMRECWYANGAARLTALRIKKTLSQLSQQEGIKM |
| SEQ ID | Human | LQCFCHLCTKDNFTCVTDGLCFVSVTETTDKVIHNSMCIAEIDLIPRD |
| NO: | TGFBR1 | RPFVCAPSSKTGSVTTTYCCNQDHCNKIELPTTVKSSPGLGPVELAA |
| 6398 | isoform 1 | VIAGPVCFVCISLMLMVYICHNRTVIHHRVPNEEDPSLDRPFISEGTT |
| | (P36897-1) | LKDLIYDMTTSGSGSGLPLLVQRTIARTIVLQESIGKGRFGEVWRGK |
| | | WRGEEVAVKIFSSREERSWFREAEIYQTVMLRHENILGFIAADNKDN |
| | | GTWTQLWLVSDYHEHGSLFDYLNRYTVTVEGMIKLALSTASGLAH |
| | | LHMEIVGTQGKPAIAHRDLKSKNILVKKNGTCCIADLGLAVRHDSA |
| | | TDTIDIAPNHRVGTKRYMAPEVLDDSINMKHFESFKRADIYAMGLV |
| | | FWEIARRCSIGGIHEDYQLPYYDLVPSDPSVEEMRKVVCEQKLRPNI |
| | | PNRWQSCEALRVMAKIMRECWYANGAARLTALRIKKTLSQLSQQE |
| | | GIKM |
| SEQ ID | Immature | MEAAVAAPRPRLLLLVLAAAAAAAAALLPGATALQCFCHLCTKDN |
| NO: | human | FTCVTDGLCFVSVTETTDKVIHNSMCIAEIDLIPRDRPFVCAPSSKTG |
| 6382 | TGFBR1 | SVTTTYCCNQDHCNKIELPTTGPFSVKSSPGLGPVELAAVIAGPVCF |
| | isoform 2 | VCISLMLMVYICHNRTVIHHRVPNEEDPSLDRPFISEGTTLKDLIYDM |
| | (P36897-2) | TTSGSGSGLPLLVQRTIARTIVLQESIGKGRFGEVWRGKWRGEEVAV |
| | | KIFSSREERSWFREAEIYQTVMLRHENILGFIAADNKDNGTWTQLWL |
| | | VSDYHEHGSLFDYLNRYTVTVEGMIKLALSTASGLAHLHMEIVGTQ |
| | | GKPAIAHRDLKSKNILVKKNGTCCIADLGLAVRHDSATDTIDIAPNH |
| | | RVGTKRYMAPEVLDDSINMKHFESFKRADIYAMGLVFWEIARRCSI |
| | | GGIHEDYQLPYYDLVPSDPSVEEMRKVVCEQKLRPNIPNRWQSCEA |
| | | LRVMAKIMRECWYANGAARLTALRIKKTLSQLSQQEGIKM |
| SEQ ID | Human | LQCFCHLCTKDNFTCVTDGLCFVSVTETTDKVIHNSMCIAEIDLIPRD |
| NO: | TGFBR1 | RPFVCAPSSKTGSVTTTYCCNQDHCNKIELPTTGPFSVKSSPGLGPVE |
| 6399 | isoform 2 | LAAVIAGPVCFVCISLMLMVYICHNRTVIHHRVPNEEDPSLDRPFISE |
| | (P36897-2) | GTTLKDLIYDMTTSGSGSGLPLLVQRTIARTIVLQESIGKGRFGEVW |
| | | RGKWRGEEVAVKIFSSREERSWFREAEIYQTVMLRHENILGFIAADN |
| | | KDNGTWTQLWLVSDYHEHGSLFDYLNRYTVTVEGMIKLALSTASG |
| | | LAHLHMEIVGTQGKPAIAHRDLKSKNILVKKNGTCCIADLGLAVRH |
| | | DSATDTIDIAPNHRVGTKRYMAPEVLDDSINMKHFESFKRADIYAM |
| | | GLVFWEIARRCSIGGIHEDYQLPYYDLVPSDPSVEEMRKVVCEQKLR |
| | | PNIPNRWQSCEALRVMAKIMRECWYANGAARLTALRIKKTLSQLSQ |
| | | QEGIKM |
| SEQ ID | Immature | MEAAVAAPRPRLLLLVLAAAAAAAAALLPGATALQCFCHLCTKDN |
| NO: | human | FTCVTDGLCFVSVTETTDKVIHNSMCIAEIDLIPRDRPFVCAPSSKTG |
| 6383 | TGFBR1 | SVTTTYCCNQDHCNKIELPTTGLPLLVQRTIARTIVLQESIGKGRFGE |
| | isoform 3 | VWRGKWRGEEVAVKIFSSREERSWFREAEIYQTVMLRHENILGFIA |
| | (P36897-3) | ADNKDNGTWTQLWLVSDYHEHGSLFDYLNRYTVTVEGMIKLALST |
| | | ASGLAHLHMEIVGTQGKPAIAHRDLKSKNILVKKNGTCCIADLGLA |
| | | VRHDSATDTIDIAPNHRVGTKRYMAPEVLDDSINMKHFESFKRADIY |
| | | AMGLVFWEIARRCSIGGIHEDYQLPYYDLVPSDPSVEEMRKVVCEQ |
| | | KLRPNIPNRWQSCEALRVMAKIMRECWYANGAARLTALRIKKTLS |
| | | QLSQQEGIKM |
| SEQ ID | Human | LQCFCHLCTKDNFTCVTDGLCFVSVTETTDKVIHNSMCIAEIDLIPRD |
| NO: | TGFBR1 | RPFVCAPSSKTGSVTTTYCCNQDHCNKIELPTTGLPLLVQRTIARTIV |
| 6400 | isoform 3 | LQESIGKGRFGEVWRGKWRGEEVAVKIFSSREERSWFREAEIYQTV |
| | (P36897-3) | MLRHENILGFIAADNKDNGTWTQLWLVSDYHEHGSLFDYLNRYTV |
| | | TVEGMIKLALSTASGLAHLHMEIVGTQGKPAIAHRDLKSKNILVKK |
| | | NGTCCIADLGLAVRHDSATDTIDIAPNHRVGTKRYMAPEVLDDSIN |
| | | MKHFESFKRADIYAMGLVFWEIARRCSIGGIHEDYQLPYYDLVPSDP |
| | | SVEEMRKVVCEQKLRPNIPNRWQSCEALRVMAKIMRECWYANGA |
| | | ARLTALRIKKTLSQLSQQEGIKM |
| SEQ ID | Human | LQCFCHLCTKDNFTCVTDGLCFVSVTETTDKVIHNSMCIAEIDLIPRD |
| NO: | TGFBR1 | RPFVCAPSSKTGSVTTTYCCNQDHCNKIELPTTVKSSPGLGPVEL |
| 6390 | fragment 1 | |
| SEQ ID | Human | ALQCFCHLCTKDNFTCVTDGLCFVSVTETTDKVIHNSMCIAEIDLIPR |
| NO: | TGFBR1 | DRPFVCAPSSKTGSVTTTYCCNQDHCNKIEL |
| 6391 | fragment 2 | |
| SEQ ID | Immature | MGRGLLRGLWPLHIVLWTRIASTIPPHVQKSVNNDMIVTDNNGAVK |
| NO: | human | FPQLCKFCDVRFSTCDNQKSCMSNCSITSICEKPQEVCVAVWRKND |
| 6384 | TGFBR2 | ENITLETVCHDPKLPYHDFILEDAASPKCIMKEKKKPGETFFMCSCSS |
| | isoform B | DECNDNIIFSEEYNTSNPDLLLVIFQVTGISLLPPLGVAISVIIIFYCYRV |
| | (short | NRQQKLSSTWETGKTRKLMEFSEHCAIILEDDRSDISSTCANNINHN |
| | isoform) | TELLPIELDTLVGKGRFAEVYKAKLKQNTSEQFETVAVKIFPYEEYA |
| | (P37173-1) | SWKTEKDIFSDINLKHENILQFLTAEERKTELGKQYWLITAFHAKGN |
| | | LQEYLTRHVISWEDLRKLGSSLARGIAHLHSDHTPCGRPKMPIVHRD |
| | | LKSSNILVKNDLTCCLCDFGLSLRLDPTLSVDDLANSGQVGTARYM |
| | | APEVLESRMNLENVESFKQTDVYSMALVLWEMTSRCNAVGEVKD |
| | | YEPPFGSKVREHPCVESMKDNVLRDRGRPEIPSFWLNHQGIQMVCE |
| | | TLTECWDHDPEARLTAQCVAERFSELEHLDRLSGRSCSEEKIPEDGS |
| | | LNTTK |
| SEQ ID | Human | TIPPHVQKSVNNDMIVTDNNGAVKFPQLCKFCDVRFSTCDNQKSCM |
| NO: | TGFBR2 | SNCSITSICEKPQEVCVAVWRKNDENITLETVCHDPKLPYHDFILEDA |
| 6401 | isoform B | ASPKCIMKEKKKPGETFFMCSCSSDECNDNIIFSEEYNTSNPDLLLVIF |
| | (short | QVTGISLLPPLGVAISVIIIFYCYRVNRQQKLSSTWETGKTRKLMEFS |
| | isoform) | EHCAIILEDDRSDISSTCANNINHNTELLPIELDTLVGKGRFAEVYKA |
| | (P37173-1) | KLKQNTSEQFETVAVKIFPYEEYASWKTEKDIFSDINLKHENILQFLT |
| | | AEERKTELGKQYWLITAFHAKGNLQEYLTRHVISWEDLRKLGSSLA |
| | | RGIAHLHSDHTPCGRPKMPIVHRDLKSSNILVKNDLTCCLCDFGLSL |
| | | RLDPTLSVDDLANSGQVGTARYMAPEVLESRMNLENVESFKQTDV |
| | | YSMALVLWEMTSRCNAVGEVKDYEPPFGSKVREHPCVESMKDNVL |
| | | RDRGRPEIPSFWLNHQGIQMVCETLTECWDHDPEARLTAQCVAERF |
| | | SELEHLDRLSGRSCSEEKIPEDGSLNTTK |
| SEQ ID | Immature | MGRGLLRGLWPLHIVLWTRIASTIPPHVQKSDVEMEAQKDEIICPSC |
| NO: | human | NRTAHPLRHINNDMIVTDNNGAVKFPQLCKFCDVRFSTCDNQKSCM |
| 6385 | TGFBR2 | SNCSITSICEKPQEVCVAVWRKNDENITLETVCHDPKLPYHDFILEDA |
| | isoform A | ASPKCIMKEKKKPGETFFMCSCSSDECNDNIIFSEEYNTSNPDLLLVIF |
| | (long | QVTGISLLPPLGVAISVIIIFYCYRVNRQQKLSSTWETGKTRKLMEFS |
| | isoform) | EHCAIILEDDRSDISSTCANNINHNTELLPIELDTLVGKGRFAEVYKA |
| | (P37173-2) | KLKQNTSEQFETVAVKIFPYEEYASWKTEKDIFSDINLKHENILQFLT |
| | | AEERKTELGKQYWLITAFHAKGNLQEYLTRHVISWEDLRKLGSSLA |
| | | RGIAHLHSDHTPCGRPKMPIVHRDLKSSNILVKNDLTCCLCDFGLSL |
| | | RLDPTLSVDDLANSGQVGTARYMAPEVLESRMNLENVESFKQTDV |
| | | YSMALVLWEMTSRCNAVGEVKDYEPPFGSKVREHPCVESMKDNVL |
| | | RDRGRPEIPSFWLNHQGIQMVCETLTECWDHDPEARLTAQCVAERF |
| | | SELEHLDRLSGRSCSEEKIPEDGSLNTTK |
| SEQ ID | Human | TIPPHVQKSDVEMEAQKDEIICPSCNRTAHPLRHINNDMIVTDNNGA |
| NO: | TGFBR2 | VKFPQLCKFCDVRFSTCDNQKSCMSNCSITSICEKPQEVCVAVWRK |
| 6402 | isoform A | NDENITLETVCHDPKLPYHDFILEDAASPKCIMKEKKKPGETFFMCS |
| | (long | CSSDECNDNIIFSEEYNTSNPDLLLVIFQVTGISLLPPLGVAISVIIIFYC |
| | isoform) | YRVNRQQKLSSTWETGKTRKLMEFSEHCAIILEDDRSDISSTCANNI |
| | (P37173-2) | NHNTELLPIELDTLVGKGRFAEVYKAKLKQNTSEQFETVAVKIFPYE |
| | | EYASWKTEKDIFSDINLKHENILQFLTAEERKTELGKQYWLITAFHA |
| | | KGNLQEYLTRHVISWEDLRKLGSSLARGIAHLHSDHTPCGRPKMPIV |
| | | HRDLKSSNILVKNDLTCCLCDFGLSLRLDPTLSVDDLANSGQVGTA |
| | | RYMAPEVLESRMNLENVESFKQTDVYSMALVLWEMTSRCNAVGE |
| | | VKDYEPPFGSKVREHPCVESMKDNVLRDRGRPEIPSFWLNHQGIQM |
| | | VCETLTECWDHDPEARLTAQCVAERFSELEHLDRLSGRSCSEEKIPE |
| | | DGSLNTTK |
| SEQ ID | Human | TIPPHVQKSVNNDMIVTDNNGAVKFPQLCKFCDVRFSTCDNQKSCM |
| NO: | TGFBR2 | SNCSITSICEKPQEVCVAVWRKNDENITLETVCHDPKLPYHDFILEDA |
| 6386 | fragment 1 | ASPKCIMKEKKKPGETFFMCSCSSDECNDNIIFSEEYNTSNPD |
| | (ECD of | |
| | human | |
| | TGFBR2 | |
| | isoform B) | |
| SEQ ID | Human | IPPHVQKSVNNDMIVTDNNGAVKFPQLCKFCDVRFSTCDNQKSCMS |
| NO: | TGFBR2 | NCSITSICEKPQEVCVAVWRKNDENITLETVCHDPKLPYHDFILEDA |
| 6387 | fragment 2 | ASPKCIMKEKKKPGETFFMCSCSSDECNDNIIFSEEYNTSNPD |
| SEQ ID | Human | TIPPHVQKSDVEMEAQKDEIICPSCNRTAHPLRHINNDMIVTDNNGA |
| NO: | TGFBR2 | VKFPQLCKFCDVRFSTCDNQKSCMSNCSITSICEKPQEVCVAVWRK |
| 6388 | fragment 3 | NDENITLETVCHDPKLPYHDFILEDAASPKCIMKEKKKPGETFFMCS |
| | (ECD of | CSSDECNDNIIFSEEYNTSNPD |
| | human | |
| | TGFBR2 | |
| | isoform A) | |
| SEQ ID | Human | QLCKFCDVRFSTCDNQKSCMSNCSITSICEKPQEVCVAVWRKNDENI |
| NO: | TGFBR2 | TLETVCHDPKLPYHDFILEDAASPKCIMKEKKKPGETFFMCSCSSDE |
| 6389 | fragment 4 | CNDNIIF |
| SEQ ID | Immature | MTSHYVIAIFALMSSCLATAGPEPGALCELSPVSASHPVQALMESFT |
| NO: | human | VLSGCASRGTTGLPQEVHVLNLRTAGQGPGQLQREVTLHLNPISSV |
| 6392 | TGFBR3 | HIHHKSVVFLLNSPHPLVWHLKTERLATGVSRLFLVSEGSVVQFSSA |
| | isoform 1 | NFSLTAETEERNFPHGNEHLLNWARKEYGAVTSFTELKIARNIYIKV |
| | (Q03167-1) | GEDQVFPPKCNIGKNFLSLNYLAEYLQPKAAEGCVMSSQPQNEEVH |
| | | IIELITPNSNPYSAFQVDITIDIRPSQEDLEVVKNLILILKCKKSVNWVI |
| | | KSFDVKGSLKIIAPNSIGFGKESERSMTMTKSIRDDIPSTQGNLVKWA |
| | | LDNGYSPITSYTMAPVANRFHLRLENNAEEMGDEEVHTIPPELRILL |
| | | DPGALPALQNPPIRGGEGQNGGLPFPFPDISRRVWNEEGEDGLPRPK |
| | | DPVIPSIQLFPGLREPEEVQGSVDIALSVKCDNEKMIVAVEKDSFQAS |
| | | GYSGMDVTLLDPTCKAKMNGTHFVLESPLNGCGTRPRWSALDGVV |
| | | YYNSIVIQVPALGDSSGWPDGYEDLESGDNGFPGDMDEGDASLFTR |
| | | PEIVVFNCSLQQVRNPSSFQEQPHGNITFNMELYNTDLFLVPSQGVFS |
| | | VPENGHVYVEVSVTKAEQELGFAIQTCFISPYSNPDRMSHYTIIENIC |
| | | PKDESVKFYSPKRVHFPIPQADMDKKRFSFVFKPVFNTSLLFLQCEL |
| | | TLCTKMEKHPQKLPKCVPPDEACTSLDASIIWAMMQNKKTFTKPLA |
| | | VIHHEAESKEKGPSMKEPNPISPPIFHGLDTLTVMGIAFAAFVIGALL |
| | | TGALWYIYSHTGETAGRQQVPTSPPASENSSAAHSIGSTQSTPCSSSS |
| | | TA |
| SEQ ID | Human | GPEPGALCELSPVSASHPVQALMESFTVLSGCASRGTTGLPQEVHVL |
| NO: | TGFBR3 | NLRTAGQGPGQLQREVTLHLNPISSVHIHHKSVVFLLNSPHPLVWHL |
| 6403 | isoform 1 | KTERLATGVSRLFLVSEGSVVQFSSANFSLTAETEERNFPHGNEHLL |
| | (Q03167-1) | NWARKEYGAVTSFTELKIARNIYIKVGEDQVFPPKCNIGKNFLSLNY |
| | | LAEYLQPKAAEGCVMSSQPQNEEVHIIELITPNSNPYSAFQVDITIDIR |
| | | PSQEDLEVVKNLILILKCKKSVNWVIKSFDVKGSLKIIAPNSIGFGKES |
| | | ERSMTMTKSIRDDIPSTQGNLVKWALDNGYSPITSYTMAPVANRFH |
| | | LRLENNAEEMGDEEVHTIPPELRILLDPGALPALQNPPIRGGEGQNG |
| | | GLPFPFPDISRRVWNEEGEDGLPRPKDPVIPSIQLFPGLREPEEVQGSV |
| | | DIALSVKCDNEKMIVAVEKDSFQASGYSGMDVTLLDPTCKAKMNG |
| | | THFVLESPLNGCGTRPRWSALDGVVYYNSIVIQVPALGDSSGWPDG |
| | | YEDLESGDNGFPGDMDEGDASLFTRPEIVVFNCSLQQVRNPSSFQEQ |
| | | PHGNITFNMELYNTDLFLVPSQGVFSVPENGHVYVEVSVTKAEQEL |
| | | GFAIQTCFISPYSNPDRMSHYTIIENICPKDESVKFYSPKRVHFPIPQA |
| | | DMDKKRFSFVFKPVFNTSLLFLQCELTLCTKMEKHPQKLPKCVPPD |
| | | EACTSLDASIIWAMMQNKKTFTKPLAVIHHEAESKEKGPSMKEPNPI |
| | | SPPIFHGLDTLTVMGIAFAAFVIGALLTGALWYIYSHTGETAGRQQV |
| | | PTSPPASENSSAAHSIGSTQSTPCSSSSTA |
| SEQ ID | Immature | MTSHYVIAIFALMSSCLATAGPEPGALCELSPVSASHPVQALMESFT |
| NO: | human | VLSGCASRGTTGLPQEVHVLNLRTAGQGPGQLQREVTLHLNPISSV |
| 6393 | TGFBR3 | HIHHKSVVFLLNSPHPLVWHLKTERLATGVSRLFLVSEGSVVQFSSA |
| | isoform 2 | NFSLTAETEERNFPHGNEHLLNWARKEYGAVTSFTELKIARNIYIKV |
| | (Q03167-2) | GEDQVFPPKCNIGKNFLSLNYLAEYLQPKAAEGCVMSSQPQNEEVH |
| | | IIELITPNSNPYSAFQVDITIDIRPSQEDLEVVKNLILILKCKKSVNWVI |
| | | KSFDVKGSLKIIAPNSIGFGKESERSMTMTKSIRDDIPSTQGNLVKWA |
| | | LDNGYSPITSYTMAPVANRFHLRLENNEEMGDEEVHTIPPELRILLDP |
| | | GALPALQNPPIRGGEGQNGGLPFPFPDISRRVWNEEGEDGLPRPKDP |
| | | VIPSIQLFPGLREPEEVQGSVDIALSVKCDNEKMIVAVEKDSFQASGY |
| | | SGMDVTLLDPTCKAKMNGTHFVLESPLNGCGTRPRWSALDGVVYY |
| | | NSIVIQVPALGDSSGWPDGYEDLESGDNGFPGDMDEGDASLFTRPEI |
| | | VVFNCSLQQVRNPSSFQEQPHGNITFNMELYNTDLFLVPSQGVFSVP |
| | | ENGHVYVEVSVTKAEQELGFAIQTCFISPYSNPDRMSHYTIIENICPK |
| | | DESVKFYSPKRVHFPIPQADMDKKRFSFVFKPVFNTSLLFLQCELTL |
| | | CTKMEKHPQKLPKCVPPDEACTSLDASIIWAMMQNKKTFTKPLAVI |
| | | HHEAESKEKGPSMKEPNPISPPIFHGLDTLTVMGIAFAAFVIGALLTG |
| | | ALWYIYSHTGETAGRQQVPTSPPASENSSAAHSIGSTQSTPCSSSSTA |
| SEQ ID | Human | GPEPGALCELSPVSASHPVQALMESFTVLSGCASRGTTGLPQEVHVL |
| NO: | TGFBR3 | NLRTAGQGPGQLQREVTLHLNPISSVHIHHKSVVFLLNSPHPLVWHL |
| 6404 | isoform 2 | KTERLATGVSRLFLVSEGSVVQFSSANFSLTAETEERNFPHGNEHLL |
| | (Q03167-2) | NWARKEYGAVTSFTELKIARNIYIKVGEDQVFPPKCNIGKNFLSLNY |
| | | LAEYLQPKAAEGCVMSSQPQNEEVHIIELITPNSNPYSAFQVDITIDIR |
| | | PSQEDLEVVKNLILILKCKKSVNWVIKSFDVKGSLKIIAPNSIGFGKES |
| | | ERSMTMTKSIRDDIPSTQGNLVKWALDNGYSPITSYTMAPVANRFH |
| | | LRLENNEEMGDEEVHTIPPELRILLDPGALPALQNPPIRGGEGQNGG |
| | | LPFPFPDISRRVWNEEGEDGLPRPKDPVIPSIQLFPGLREPEEVQGSVD |
| | | IALSVKCDNEKMIVAVEKDSFQASGYSGMDVTLLDPTCKAKMNGT |
| | | HFVLESPLNGCGTRPRWSALDGVVYYNSIVIQVPALGDSSGWPDGY |
| | | EDLESGDNGFPGDMDEGDASLFTRPEIVVFNCSLQQVRNPSSFQEQP |
| | | HGNITFNMELYNTDLFLVPSQGVFSVPENGHVYVEVSVTKAEQELG |
| | | FAIQTCFISPYSNPDRMSHYTIIENICPKDESVKFYSPKRVHFPIPQAD |
| | | MDKKRFSFVFKPVFNTSLLFLQCELTLCTKMEKHPQKLPKCVPPDE |
| | | ACTSLDASIIWAMMQNKKTFTKPLAVIHHEAESKEKGPSMKEPNPIS |
| | | PPIFHGLDTLTVMGIAFAAFVIGALLTGALWYIYSHTGETAGRQQVP |
| | | TSPPASENSSAAHSIGSTQSTPCSSSSTA |
| SEQ ID | Human | GPEPGALCELSPVSASHPVQALMESFTVLSGCASRGTTGLPQEVHVL |
| NO: | TGFBR3 | NLRTAGQGPGQLQREVTLHLNPISSVHIHHKSVVFLLNSPHPLVWHL |
| 6394 | fragment 1 | KTERLATGVSRLFLVSEGSVVQFSSANFSLTAETEERNFPHGNEHLL |
| | | NWARKEYGAVTSFTELKIARNIYIKVGEDQVFPPKCNIGKNFLSLNY |
| | | LAEYLQPKAAEGCVMSSQPQNEEVHIIELITPNSNPYSAFQVDITIDIR |
| | | PSQEDLEVVKNLILILKCKKSVNWVIKSFDVKGSLKIIAPNSIGFGKES |
| | | ERSMTMTKSIRDDIPSTQGNLVKWALDNGYSPITSYTMAPVANRFH |
| | | LRLENNAEEMGDEEVHTIPPELRILLDPGALPALQNPPIRGGEGQNG |
| | | GLPFPFPDISRRVWNEEGEDGLPRPKDPVIPSIQLFPGLREPEEVQGSV |
| | | DIALSVKCDNEKMIVAVEKDSFQASGYSGMDVTLLDPTCKAKMNG |
| | | THFVLESPLNGCGTRPRWSALDGVVYYNSIVIQVPALGDSSGWPDG |
| | | YEDLESGDNGFPGDMDEGDASLFTRPEIVVFNCSLQQVRNPSSFQEQ |
| | | PHGNITFNMELYNTDLFLVPSQGVFSVPENGHVYVEVSVTKAEQEL |
| | | GFAIQTCFISPYSNPDRMSHYTIIENICPKDESVKFYSPKRVHFPIPQA |
| | | DMDKKRFSFVFKPVFNTSLLFLQCELTLCTKMEKHPQKLPKCVPPD |
| | | EACTSLDASIIWAMMQNKKTFTKPLAVIHHEAESKEKGPSMKEPNPI |
| | | SPPIFHGLDTLTV |
| SEQ ID | hCH1- | ASTKGPSVFPLAPSSKSTSGGTAALGCLVKDYFPEPVTVSWNSGALT |
| NO: | hFc_Hole- | SGVHTFPAVLQSSGLYSLSSVVTVPSSSLGTQTYICNVNHKPSNTKV |
| 6405 | 3x4GS- | DKRVEPKSCDKTHTCPPCPAPELLGGPSVFLFPPKPKDTLMISRTPEV |
| | TGFbR2 | TCVVVDVSHEDPEVKFNWYVDGVEVHNAKTKPREEQYNSTYRVVS |
| | | VLTVLHQDWLNGKEYKCKVSNKALPAPIEKTISKAKGQPREPQVCT |
| | | LPPSREEMTKNQVSLSCAVKGFYPSDIAVEWESNGQPENNYKTTPP |
| | | VLDSDGSFFLVSKLTVDKSRWQQGNVFSCSVMHEALHNHYTQKSL |
| | | SLSPGXGGGGSGGGGSGGGGSIPPHVQKSVNNDMIVTDNNGAVKFP |
| | | QLCKFCDVRFSTCDNQKSCMSNCSITSICEKPQEVCVAVWRKNDENI |
| | | TLETVCHDPKLPYHDFILEDAASPKCIMKEKKKPGETFFMCSCSSDE |
| | | CNDNIIFSEEYNTSNPD, wherein X is K or absent |
| SEQ ID | hCH1- | ASTKGPSVFPLAPSSKSTSGGTAALGCLVKDYFPEPVTVSWNSGALT |
| NO: | hFc_Knob- | SGVHTFPAVLQSSGLYSLSSVVTVPSSSLGTQTYICNVNHKPSNTKV |
| 6406 | 3x4GS- | DKRVEPKSCDKTHTCPPCPAPELLGGPSVFLFPPKPKDTLMISRTPEV |
| | TGFbR2 | TCVVVDVSHEDPEVKFNWYVDGVEVHNAKTKPREEQYNSTYRVVS |
| | | VLTVLHQDWLNGKEYKCKVSNKALPAPIEKTISKAKGQPREPQVYT |
| | | LPPCREEMTKNQVSLWCLVKGFYPSDIAVEWESNGQPENNYKTTPP |
| | | VLDSDGSFFLYSKLTVDKSRWQQGNVFSCSVMHEALHNHYTQKSL |
| | | SLSPGXGGGGSGGGGSGGGGSIPPHVQKSVNNDMIVTDNNGAVKFP |
| | | QLCKFCDVRFSTCDNQKSCMSNCSITSICEKPQEVCVAVWRKNDENI |
| | | TLETVCHDPKLPYHDFILEDAASPKCIMKEKKKPGETFFMCSCSSDE |
| | | CNDNIIFSEEYNTSNPD, wherein X is K or absent |
| SEQ ID | hFc_Hole- | DKTHTCPPCPAPELLGGPSVFLFPPKPKDTLMISRTPEVTCVVVDVSH |
| NO: | 3x4GS- | EDPEVKFNWYVDGVEVHNAKTKPREEQYNSTYRVVSVLTVLHQD |
| 6407 | TGFbR2 | WLNGKEYKCKVSNKALPAPIEKTISKAKGQPREPQVCTLPPSREEMT |
| | | KNQVSLSCAVKGFYPSDIAVEWESNGQPENNYKTTPPVLDSDGSFFL |
| | | VSKLTVDKSRWQQGNVFSCSVMHEALHNHYTQKSLSLSPGXGGGG |
| | | SGGGGSGGGGSIPPHVQKSVNNDMIVTDNNGAVKFPQLCKFCDVRF |
| | | STCDNQKSCMSNCSITSICEKPQEVCVAVWRKNDENITLETVCHDPK |
| | | LPYHDFILEDAASPKCIMKEKKKPGETFFMCSCSSDECNDNIIFSEEY |
| | | NTSNPD, wherein X is K or absent |
| SEQ ID | hFc_Knob- | DKTHTCPPCPAPELLGGPSVFLFPPKPKDTLMISRTPEVTCVVVDVSH |
| NO: | 3x4GS- | EDPEVKFNWYVDGVEVHNAKTKPREEQYNSTYRVVSVLTVLHQD |
| 6408 | TGFbR2 | WLNGKEYKCKVSNKALPAPIEKTISKAKGQPREPQVYTLPPCREEM |
| | | TKNQVSLWCLVKGFYPSDIAVEWESNGQPENNYKTTPPVLDSDGSF |
| | | FLYSKLTVDKSRWQQGNVFSCSVMHEALHNHYTQKSLSLSPGXGG |
| | | GGSGGGGSGGGGSIPPHVQKSVNNDMIVTDNNGAVKFPQLCKFCD |
| | | VRFSTCDNQKSCMSNCSITSICEKPQEVCVAVWRKNDENITLETVCH |
| | | DPKLPYHDFILEDAASPKCIMKEKKKPGETFFMCSCSSDECNDNIIFS |
| | | EEYNTSNPD, wherein X is K or absent |
| SEQ ID | TGFbR2- | IPPHVQKSVNNDMIVTDNNGAVKFPQLCKFCDVRFSTCDNQKSCMS |
| NO: | 3x4GS- | NCSITSICEKPQEVCVAVWRKNDENITLETVCHDPKLPYHDFILEDA |
| 6409 | hCH1- | ASPKCIMKEKKKPGETFFMCSCSSDECNDNIIFSEEYNTSNPDGGGGS |
| | hFc_Hole | GGGGSGGGGSASTKGPSVFPLAPSSKSTSGGTAALGCLVKDYFPEPV |
| | | TVSWNSGALTSGVHTFPAVLQSSGLYSLSSVVTVPSSSLGTQTYICN |
| | | VNHKPSNTKVDKRVEPKSCDKTHTCPPCPAPELLGGPSVFLFPPKPK |
| | | DTLMISRTPEVTCVVVDVSHEDPEVKFNWYVDGVEVHNAKTKPRE |
| | | EQYNSTYRVVSVLTVLHQDWLNGKEYKCKVSNKALPAPIEKTISKA |
| | | KGQPREPQVCTLPPSREEMTKNQVSLSCAVKGFYPSDIAVEWESNG |
| | | QPENNYKTTPPVLDSDGSFFLVSKLTVDKSRWQQGNVFSCSVMHEA |
| | | LHNHYTQKSLSLSPGX, wherein X is K or absent |
| SEQ ID | TGFbR2- | IPPHVQKSVNNDMIVTDNNGAVKFPQLCKFCDVRFSTCDNQKSCMS |
| NO: | 3x4GS- | NCSITSICEKPQEVCVAVWRKNDENITLETVCHDPKLPYHDFILEDA |
| 6410 | hCH1- | ASPKCIMKEKKKPGETFFMCSCSSDECNDNIIFSEEYNTSNPDGGGGS |
| | hFc_Knob | GGGGSGGGGSASTKGPSVFPLAPSSKSTSGGTAALGCLVKDYFPEPV |
| | | TVSWNSGALTSGVHTFPAVLQSSGLYSLSSVVTVPSSSLGTQTYICN |
| | | VNHKPSNTKVDKRVEPKSCDKTHTCPPCPAPELLGGPSVFLFPPKPK |
| | | DTLMISRTPEVTCVVVDVSHEDPEVKFNWYVDGVEVHNAKTKPRE |
| | | EQYNSTYRVVSVLTVLHQDWLNGKEYKCKVSNKALPAPIEKTISKA |
| | | KGQPREPQVYTLPPCREEMTKNQVSLWCLVKGFYPSDIAVEWESNG |
| | | QPENNYKTTPPVLDSDGSFFLYSKLTVDKSRWQQGNVFSCSVMHEA |
| | | LHNHYTQKSLSLSPGX, wherein X is K or absent |
| SEQ ID | TGFbR2- | IPPHVQKSVNNDMIVTDNNGAVKFPQLCKFCDVRFSTCDNQKSCMS |
| NO: | 3x4GS- | NCSITSICEKPQEVCVAVWRKNDENITLETVCHDPKLPYHDFILEDA |
| 6411 | hCLIg_vl | ASPKCIMKEKKKPGETFFMCSCSSDECNDNIIFSEEYNTSNPDGGGGS |
| | | GGGGSGGGGSGQPKANPTVTLFPPSSEELQANKATLVCLISDFYPGA |
| | | VTVAWKADGSPVKAGVETTKPSKQSNNKYAASSYLSLTPEQWKSH |
| | | RSYSCQVTHEGSTVEKTVAPTECS |
| SEQ ID | TGFβR2- | IPPHVQKSVNNDMIVTDNNGAVKFPQLCKFCDVRFSTCDNQKSCMS |
| NO: | 3x4GS- | NCSITSICEKPQEVCVAVWRKNDENITLETVCHDPKLPYHDFILEDA |
| 6412 | hCLIg_vk | ASPKCIMKEKKKPGETFFMCSCSSDECNDNIIFSEEYNTSNPDGGGGS |
| | | GGGGSGGGGSRTVAAPSVFIFPPSDEQLKSGTASVVCLLNNFYPREA |
| | | KVQWKVDNALQSGNSQESVTEQDSKDSTYSLSSTLTLSKADYEKH |
| | | KVYACEVTHQGLSSPVTKSFNRGEC |

### Stromal Modifying Moieties

Solid tumors have a distinct structure that mimics that of normal tissues and comprises two distinct but interdependent compartments: the parenchyma (neoplastic cells) and the stroma that the neoplastic cells induce and in which they are dispersed. All tumors have stroma and require stroma for nutritional support and for the removal of waste products. In the case of tumors which grow as cell suspensions (e.g., leukemias, ascites tumors), the blood plasma serves as stroma (Connolly J L et al. Tumor Structure and Tumor Stroma Generation. In: Kufe D W et al., editors. Holland-Frei Cancer Medicine. 6th edition. Hamilton: BC Decker; 2003). The stroma includes a variety of cell types, including fibroblasts/myofibroblasts, glial, epithelial, fat, vascular, smooth muscle, and immune cells along with extracellular matrix (ECM) and extracellular molecules (Li Hanchen et al. Tumor Microenvironment: The Role of the Tumor Stroma in Cancer. J of Cellular Biochemistry 101: 805-815 (2007)).

Stromal modifying moieties described herein include moieties (e.g., proteins, e.g., enzymes) capable of degrading a component of the stroma, e.g., an ECM component, e.g., a glycosaminoglycan, e.g., hyaluronan (also known as hyaluronic acid or HA), chondroitin sulfate, chondroitin, dermatan sulfate, heparin sulfate, heparin, entactin, tenascin, aggrecan and keratin sulfate; or an extracellular protein, e.g., collagen, laminin, elastin, fibrinogen, fibronectin, and vitronectin.

### Stromal Modifying Enzymes

In some embodiments, the stromal modifying moiety is an enzyme. For example, the stromal modifying moiety can include, but is not limited to a hyaluronidase, a collagenase, a chondroitinase, a matrix metalloproteinase (e.g., macrophage metalloelastase).

Hyaluronidases

Hyaluronidases are a group of neutral- and acid-active enzymes found throughout the animal kingdom. Hyaluronidases vary with respect to substrate specificity, and mechanism of action. There are three general classes of hyaluronidases: (1) Mammalian-type hyaluronidases, (EC 3.2.1.35) which are endo-beta-N-acetylhexosaminidases with tetrasaccharides and hexasaccharides as the major end products. They have both hydrolytic and transglycosidase activities, and can degrade hyaluronan and chondroitin sulfates; (2) Bacterial hyaluronidases (EC 4.2.99.1) degrade hyaluronan and, and to various extents, chondroitin sulfate and dermatan sulfate. They are endo-beta-N-acetylhexosaminidases that operate by a beta elimination reaction that yields primarily disaccharide end products; (3) Hyaluronidases (EC 3.2.1.36) from leeches, other parasites, and crustaceans are endo-beta-glucuronidases that generate tetrasaccharide and hexasaccharide end products through hydrolysis of the beta 1-3 linkage.

Mammalian hyaluronidases can be further divided into two groups: (1) neutral active and (2) acid active enzymes. There are six hyaluronidase-like genes in the human genome, HYAL1, HYAL2, HYAL3 HYAL4 HYALP1 and PH20/SPAM1. HYALP1 is a pseudogene, and HYAL3 has not been shown to possess enzyme activity toward any known substrates. HYAL4 is a chondroitinase and lacks activity towards hyaluronan. HYAL1 is the prototypical acid-active enzyme and PH20 is the prototypical neutral-active enzyme. Acid active hyaluronidases, such as HYAL1 and HYAL2 lack catalytic activity at neutral pH. For example, HYAL1 has no catalytic activity in vitro over pH 4.5 (Frost and Stem, “A Microtiter-Based Assay for Hyaluronidase Activity Not Requiring Specialized Reagents”, Analytical Biochemistry, vol. 251, pp. 263-269 (1997). HYAL2 is an acid active enzyme with a very low specific activity in vitro.

In some embodiments the hyaluronidase is a mammalian hyaluronidase. In some embodiments the hyaluronidase is a recombinant human hyaluronidase. In some embodiments, the hyaluronidase is a neutral active hyaluronidase. In some embodiments, the hyaluronidase is a neutral active soluble hyaluronidase. In some embodiments, the hyaluronidase is a recombinant PH20 neutral-active enzyme. In some embodiments, the hyaluronidase is a recombinant PH20 neutral-active soluble enzyme. In some embodiments the hyaluronidase is glycosylated. In some embodiments, the hyaluronidase possesses at least one N-linked glycan. A recombinant hyaluronidase can be produced using conventional methods known to those of skill in the art, e.g., U.S. Pat. No. 7,767,429, the entire contents of which are incorporated by reference herein.

In some embodiments the hyaluronidase is rHuPH20 (also referred to as Hylenex®; presently manufactured by Halozyme; approved by the FDA in 2005 (see e.g., Scodeller P (2014) Hyaluronidase and other Extracellular Matrix Degrading Enzymes for Cancer Therapy: New Uses and Nano-Formulations. J Carcinog Mutage 5:178; U.S. Pat. Nos. 7,767,429; 8,202,517; 7,431,380; 8,450,470; 8,772,246; 8,580,252, the entire contents of each of which is incorporated by reference herein). rHuPH20 is produced by genetically engineered CHO cells containing a DNA plasmid encoding for a soluble fragment of human hyaluronidase PH20. In some embodiments the hyaluronidase is glycosylated. In some embodiments, the hyaluronidase possesses at least one N-linked glycan. A recombinant hyaluronidase can be produced using conventional methods known to those of skill in the art, e.g., U.S. Pat. No. 7,767,429, the entire contents of which are incorporated by reference herein. In some embodiments, rHuPH20 has a sequence at least 95% (e.g., at least 96° 98% 99%, 100%) identical to the amino acid sequence of

(SEQ ID NO: 6213)
LNFRAPPVIPNVPFLWAWNAPSEFCLGKFDEPLDMSLFSFIGSPRINAT
GQGVTIFYVDRLGYYPYIDSITGVTVNGGIPQKISLQDHLDKAKKDITF
YMPVDNLGMAVIDWEEWRPTWARNWKPKDVYKNRSIELVQQQNVQLSLT
EATEKAKQEFEKAGKDFLVETIKLGKLLRPNHLWGYYLFPDCYNHHYKK
PGYNGSCFNVEIKRNDDLSWLWNESTALYPSIYLNTQQSPVAATLYVRN
RVREAIRVSKIPDAKSPLPVFAYTRIVFTDQVLKFLSQDELVYTFGETV
ALGASGIVIWGTLSIMRSMKSCLLLDNYMETILNPYIINVTLAAKMCSQ
VLCQEQGVCIRKNWNSSDYLHLNPDNFAIQLEKGGKFTVRGKPTLEDLE
QFSEKFYCSCYSTLSCKEKADVKDTDAVDVCIADGVCIDAFLKPPMETE
EPQIFYNASPSTLS.

In any of the methods provided herein, the anti-hyaluronan agent can be an agent that degrades hyaluronan or can be an agent that inhibits the synthesis of hyaluronan. For example, the anti-hyaluronan agent can be a hyaluronan degrading enzyme. In another example, the anti-hyaluronan agent or is an agent that inhibits hyaluronan synthesis. For example, the anti-hyaluronan agent is an agent that inhibits hyaluronan synthesis such as a sense or antisense nucleic acid molecule against an HA synthase or is a small molecule drug. For example, an anti-hyaluronan agent is 4-methylumbelliferone (MU) or a derivative thereof, or leflunomide or a derivative thereof. Such derivatives include, for example, a derivative of 4-methylumbelliferone (MU) that is 6,7-dihydroxy-4-methyl coumarin or 5,7-dihydroxy-4-methyl coumarin.

In further examples of the methods provided herein, the hyaluronan degrading enzyme is a hyaluronidase. In some examples, the hyaluronan-degrading enzyme is a PH20 hyaluronidase or truncated form thereof to lacking a C-terminal glycosylphosphatidylinositol (GPI) attachment site or a portion of the GPI attachment site. In specific examples, the hyaluronidase is a PH20 selected from a human, monkey, bovine, ovine, rat, mouse or guinea pig PH20. For example, the hyaluronan-degrading enzyme is a human PH20 hyaluronidase that is neutral active and N-glycosylated and is selected from among (a) a hyaluronidase polypeptide that is a full-length PH20 or is a C-terminal truncated form of the PH20, wherein the truncated form includes at least amino acid residues 36-464 of SEQ ID NO: 6213, such as 36-481, 36-482, 36-483, where the full-length PH20 has the sequence of amino acids set forth in SEQ ID NO: 6213; or (b) a hyaluronidase polypeptide comprising a sequence of amino acids having at least 85%, 86%, 87%, 88%, 89%, 90%, 91%, 92%, 93%, 94%, 95%, 96%, 97%, 98%, 99% or more sequence identity with the polypeptide or truncated form of sequence of amino acids set forth in SEQ ID NO: 6213; or (c) a hyaluronidase polypeptide of (a) or (b) comprising amino acid substitutions, whereby the hyaluronidase polypeptide has a sequence of amino acids having at least 85%, 86%, 87%, 88%, 89%, 90%, 91%, 92%, 93%, 94%, 95%, 96%, 97%, 98%, 99% or more sequence identity with the polypeptide set forth in SEQ ID NO: 6213 or the with the corresponding truncated forms thereof. In exemplary examples, the hyaluronan-degrading enzyme is a PH20 that comprises a composition designated rHuPH20.

In other examples, the anti-hyaluronan agent is a hyaluronan degrading enzyme that is modified by conjugation to a polymer. The polymer can be a PEG and the anti-hyaluronan agent a PEGylated hyaluronan degrading enzyme. Hence, in some examples of the methods provided herein the hyaluronan-degrading enzyme is modified by conjugation to a polymer. For example, the hyaluronan-degrading enzyme is conjugated to a PEG, thus the hyaluronan degrading enzyme is PEGylated. In an exemplary example, the hyaluronan-degrading enzyme is a PEGylated PH20 enzyme (PEGPH20). In the methods provided herein, the corticosteroid can be a glucocorticoid that is selected from among cortisones, dexamethasones, hydrocortisones, methylprednisolones, prednisolones and prednisones.

Chondroitinases

Chondroitinases are enzymes found throughout the animal kingdom which degrade glycosaminoglycans, specifically chondroitins and chondroitin sulfates, through an endoglycosidase reaction. In some embodiments the chondroitinase is a mammalian chondroitinase. In some embodiments the chondroitinase is a recombinant human chondroitinase. In some embodiments the chondroitinase is HYAL4. Other exemplary chondroitinases include chondroitinase ABC (derived from Proteus vulgaris; Japanese Patent Application Laid-open No 6-153947, T. Yamagata et al. J. Biol. Chem., 243, 1523 (1968), S. Suzuki et al, J. Biol. Chem., 243, 1543 (1968)), chondroitinase AC (derived from Flavobacterium heparinum; T. Yamagata et al., J. Biol. Chem., 243, 1523 (1968)), chondroitinase AC II (derived from Arthrobacter aurescens; K. Hiyama, and S. Okada, J. Biol. Chem., 250, 1824 (1975), K. Hiyama and S. Okada, J. Biochem. (Tokyo), 80, 1201 (1976)), Hyaluronidase ACIII (derived from Flavobacterium sp. Hp102; Hirofumi Miyazono et al., Seikagaku, 61, 1023 (1989)), chondroitinase B (derived from Flavobacterium heparinum; Y. M. Michelacci and C. P. Dietrich, Biochem. Biophys. Res. Commun., 56, 973 (1974), Y. M. Michelacci and C. P. Dietrich, Biochem. J., 151, 121 (1975), Kenichi Maeyama et al, Seikagaku, 57, 1189 (1985)), chondroitinase C (derived from Flavobacterium sp. Hp102; Hirofumi Miyazono et al, Seikagaku, 61, 1023 (1939)), and the like.

Matrix Metalloproteinases

Matrix metalloproteases (MMPs) are zinc-dependent endopeptidases that are the major proteases involved in extracellular matrix (ECM) degradation. MMPs are capable of degrading a wide range of extracellular molecules and a number of bioactive molecules. Twenty-four MMP genes have been identified in humans, which can be organized into six groups based on domain organization and substrate preference: Collagenases (MMP-1, -8 and -13), Gelatinases (MMP-2 and MMP-9), Stromelysins (MMP-3, -10 and -11), Matrilysin (MMP-7 and MMP-26), Membrane-type (MT)-MMPs (MMP-14, -15, -16, -17, -24 and -25) and others (MMP-12, -19, -20, -21, -23, -27 and -28). In some embodiments, the stromal modifying moiety is a human recombinant MMP (e.g., MMP-1, -2, -3, -4, -5, -6, -7, -8, -9, 10, -11, -12, -13, -14, 15, -15, -17, -18, -19, 20, -21, -22, -23, or -24).

Collagenases

The three mammalian collagenases (MMP-1, -8, and -13) are the principal secreted endopeptidases capable of cleaving collagenous extracellular matrix. In addition to fibrillar collagens, collagenases can cleave several other matrix and non-matrix proteins including growth factors. Collagenases are synthesized as inactive pro-forms, and once activated, their activity is inhibited by specific tissue inhibitors of metalloproteinases, TIMPs, as well as by non-specific proteinase inhibitors (Ala-aho R et al. Biochimie. Collagenases in cancer. 2005 Mar-Apr; 87(3-4):273-86). In some embodiments, the stromal modifying moiety is a collagenase. In some embodiments, the collagenase is a human recombinant collagenase. In some embodiments, the collagenase is MMP-1. In some embodiments, the collagenase is MMP-8. In some embodiments, the collagenase is MMP-13.

Macrophage Metalloelastase

Macrophage metalloelastase (MME), also known as MMP-12, is a member of the stromelysin subgroup of MMPs and catalyzes the hydrolysis of soluble and insoluble elastin and a broad selection of matrix and nonmatrix substrates including type IV collagen, fibronectin, laminin, vitronectin, entactin, heparan, and chondroitin sulfates (Erja Kerkelä et al. Journal of Investigative Dermatology (2000) 114, 1113-1119; doi: 10.1046/j.1523-1747.2000.00993). In some embodiments, the stromal modifying moiety is a MME. In some embodiments, the MME is a human recombinant MME. In some embodiments, the MME is MMP-12.

### Additional Stromal Modifying Moieties

In some embodiments, the stromal modifying moiety causes one or more of: decreases the level or production of a stromal or extracellular matrix (ECM) component; decreases tumor fibrosis; increases interstitial tumor transport; improves tumor perfusion; expands the tumor microvasculature; decreases interstitial fluid pressure (IFP) in a tumor; or decreases or enhances penetration or diffusion of an agent, e.g., a cancer therapeutic or a cellular therapy, into a tumor or tumor vasculature.

In some embodiments, the stromal or ECM component decreased is chosen from a glycosaminoglycan or an extracellular protein, or a combination thereof. In some embodiments, the glycosaminoglycan is chosen from hyaluronan (also known as hyaluronic acid or HA), chondroitin sulfate, chondroitin, dermatan sulfate, heparin, heparin sulfate, entactin, tenascin, aggrecan and keratin sulfate. In some embodiments, the extracellular protein is chosen from collagen, laminin, elastin, fibrinogen, fibronectin, or vitronectin. In some embodiments, the stromal modifying moiety includes an enzyme molecule that degrades a tumor stroma or extracellular matrix (ECM). In some embodiments, the enzyme molecule is chosen from a hyaluronidase molecule, a collagenase molecule, a chondroitinase molecule, a matrix metalloproteinase molecule (e.g., macrophage metalloelastase), or a variant (e.g., a fragment) of any of the aforesaid. The term “enzyme molecule” includes a full length, a fragment or a variant of the enzyme, e.g., an enzyme variant that retains at least one functional property of the naturally-occurring enzyme.

In some embodiments, the stromal modifying moiety decreases the level or production of hyaluronic acid. In other embodiments, the stromal modifying moiety comprises a hyaluronan degrading enzyme, an agent that inhibits hyaluronan synthesis, or an antibody molecule against hyaluronic acid.

In some embodiments, the hyaluronan degrading enzyme is a hyaluronidase molecule, e.g., a full length or a variant (e.g., fragment thereof) thereof. In some embodiments, the hyaluronan degrading enzyme is active in neutral or acidic pH, e.g., pH of about 4-5. In some embodiments, the hyaluronidase molecule is a mammalian hyaluronidase molecule, e.g., a recombinant human hyaluronidase molecule, e.g., a full length or a variant (e.g., fragment thereof, e.g., a truncated form) thereof. In some embodiments, the hyaluronidase molecule is chosen from HYAL1, HYAL2, or PH-20/SPAM1, or a variant thereof (e.g., a truncated form thereof). In some embodiments, the truncated form lacks a C-terminal glycosylphosphatidylinositol (GPI) attachment site or a portion of the GPI attachment site. In some embodiments, the hyaluronidase molecule is glycosylated, e.g., comprises at least one N-linked glycan.

In some embodiments, the hyaluronidase molecule comprises the amino acid sequence:

(SEQ ID NO: 6213)
LNFRAPPVIPNVPFLWAWNAPSEFCLGKFDEPLDMSLFSFIGSPRINAT
GQGVTIFYVDRLGYYPYIDSITGVTVNGGIPQKISLQDHLDKAKKDITF
YMPVDNLGMAVIDWEEWRPTWARNWKPKDVYKNRSIELVQQQNVQLSLT
EATEKAKQEFEKAGKDFLVETIKLGKLLRPNHLWGYYLFPDCYNHHYKK
PGYNGSCFNVEIKRNDDLSWLWNESTALYPSIYLNTQQSPVAATLYVRN
RVREAIRVSKIPDAKSPLPVFAYTRIVFTDQVLKFLSQDELVYTFGETV
ALGASGIVIWGTLSIMRSMKSCLLLDNYMETILNPYIINVTLAAKMCSQ
VLCQEQGVCIRKNWNSSDYLHLNPDNFAIQLEKGGKFTVRGKPTLEDLE
QFSEKFYCSCYSTLSCKEKADVKDTDAVDVCIADGVCIDAFLKPPMETE
EPQIFYNASPSTLS,

or a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 6213.

In some embodiments, the hyaluronidase molecule comprises:
(i) the amino acid sequence of 36-464 of SEQ ID NO: 6213;
(ii) the amino acid sequence of 36481, 36482, or 36-483 of PH20, wherein PH20 has the sequence of amino acids set forth in SEQ ID NO: 6213; or
(iii) an amino acid sequence having at least 95% to 100% sequence identity to the polypeptide or truncated form of sequence of amino acids set forth in SEQ ID NO: 6213; or
(iv) an amino acid sequence having 30, 20, 10, 5 or fewer amino acid substitutions to the amino acid sequence set forth in SEQ ID NO: 6213. In some embodiments, the hyaluronidase molecule comprises an amino acid sequence at least 95% (e.g., at least 95%, 96%, 97%, 98%, 99%, 100%) identical to the amino acid sequence of SEQ ID NO: 6213. In some embodiments, the hyaluronidase molecule is encoded by a nucleotide sequence at least 95% (e.g., at least 96%, 97%, 98%, 99%, 100%) identical to the nucleotide sequence of SEQ ID NO: 6213.

In some embodiments, the hyaluronidase molecule is PH20, e.g., rHuPH20. In some embodiments, the hyaluronidase molecule is HYAL1 and comprises the amino acid sequence:

(SEQ ID NO: 6218)
FRGPLLPNRPFTTVWNANTQWCLERHGVDVDVSVFDVVANPGQTFRGPD
MTIFYSSQGTYPYYTPTGEPVFGGLPQNASLIAHLARTFQDILAAIPAP
RSRALVQAQHPDWPAPQVEAVAQDQFQGAARAWMAGTLQLGRALRPRGL
WDFSGLAVIDWEAWRPRWAFNWDTKDIYRQGFYGFPDCYNYDFLSPNYT
GQCPSGIRAQNDQLGWLWGQSRALYPSIYMPAVLEGTGKSQMYVQHRVA
EAFRVAVAAGDPNLPVLPYVQIFYDTTNHFLPLDELEHSLGESAAQGAA
GVVLWVSWENTRTKESCQAIKEYMDTTLGPFILNVTSGALLCSQALCSG
HGRCVRRTSHPKALLLLNPASFSIQLTPGGGPLSLRGALSLEDQAQMAV
EFKCRCYPGWQAPWCERKSMW,

or a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 6218.

In some embodiments, the hyaluronan degrading enzyme, e.g., the hyaluronidase molecule, further comprises a polymer, e.g., is conjugated to a polymer, e.g., PEG. In some embodiments, the hyaluronan-degrading enzyme is a PEGylated PH20 enzyme (PEGPH20). In some embodiments, the hyaluronan degrading enzyme, e.g., the hyaluronidase molecule, further comprises an immunoglobulin chain constant region (e.g., Fc region) chosen from, e.g., the heavy chain constant regions of IgG1, IgG2, IgG3, and IgG4, more particularly, the heavy chain constant region of human IgG1, IgG2, IgG3, or IgG4. In some embodiments, the immunoglobulin constant region (e.g., the Fc region) is linked, e.g., covalently linked to, the hyaluronan degrading enzyme, e.g., the hyaluronidase molecule. In some embodiments, the immunoglobulin chain constant region (e.g., Fc region) is altered, e.g., mutated, to increase or decrease one or more of: Fe receptor binding, antibody glycosylation, the number of cysteine residues, effector cell function, or complement function. In some embodiments, the hyaluronan degrading enzyme, e.g., the hyaluronidase molecule forms a dimer.

In some embodiments, the stromal modifying moiety comprises an inhibitor of the synthesis of hyaluronan, e.g., an HA synthase. In some embodiments, the inhibitor comprises a sense or an antisense nucleic acid molecule against an HA synthase or is a small molecule drug. In some embodiments, the inhibitor is 4-methylumbelliferone (MU) or a derivative thereof (e.g., 6,7-dihydroxy-4-methyl coumarin or 5,7-dihydroxy-4-methyl coumarin), or leflunomide or a derivative thereof.

In some embodiments, the stromal modifying moiety comprises antibody molecule against hyaluronic acid.

In some embodiments, the stromal modifying moiety comprises a collagenase molecule, e.g., a mammalian collagenase molecule, or a variant (e.g., fragment) thereof. In some embodiments, the collagenase molecule is collagenase molecule IV, e.g., comprising the amino acid sequence of:

(SEQ ID NO: 6219)
YNFFPRKPKWDKNQITYRIIGYTPDLDPETVDDAFARAFQVWSDVTPLR
FSRIHDGEADIMINFGRWEHGDGYPFDGKDGLLAHAFAPGTGVGGDSHF
DDDELWTLGEGQVVRVKYGNADGEYCKFPFLFNGKEYNSCTDTGRSDGF
LWCSTTYNFEKDGKYGFCPHEALFTMGGNAEGQPCKFPFRFQGTSYDSC
TTEGRTDGYRWCGTTEDYDRDKKYGFCPETAMSTVGGNSEGAPCVFPFT
FLGNKYESCTSAGRSDGKMWCATTANYDDDRKWGFCPDQGYSLFLVAAH
EFGHAMGLEHSQDPGALMAPIYTYTKNFRLSQDDIKGIQELYGASPDID
LGTGPTPTLGPVTPEICKQDIVFDGIAQIRGEIFFFKDRFIWRTVTPRD
KPMGPLLVATFWPELPEKIDAVYEAPQEEKAVFFAGNEYWIYSASTLER
GYPKPLTSLGLPPDVQRVDAAFNWSKNKKTYIFAGDKFWRYNEVKKKMD
PGFPKLIADAWNAIPDNLDAVVDLQGGGHSYFFKGAYYLKLENQSLKSV
KFGSIKSDWLGC,

or a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 6219.

### Targeting Moieties

In some embodiments, the multispecific and/or multifunctional molecules disclosed herein comprise a tumor-targeting moiety. In some embodiments, the tumor-targeting moiety targets (e.g., binds to) a tumor antigen selected from: G6B, CD34, CD41, P-selectin, Clec2, cKIT, FLT3, MPL, ITGB3, ITGB2, GP5, GP6, GP9, GP1BA, DSC2, FCGR2A, TNFRSF10A, TNFRSF10B, or TM4SF1. In some embodiments, the tumor-targeting moiety targets (e.g., binds to) G6B.

G6B refers to MPIG6B, also known as megakaryocyte and platelet inhibitory receptor G6b or C6orf25. Swiss-Prot accession number 095866 provides exemplary human G6B amino acid sequences. In some embodiments, G6B or G6B molecule is a naturally-existing G6B or a functional variant or fragment thereof.

CD34 refers to hematopoietic progenitor cell antigen CD34. Swiss-Prot accession number P28906 provides exemplary human CD34 amino acid sequences. In some embodiments, CD34 or CD34 molecule is a naturally-existing CD34 or a functional variant or fragment thereof.

CD41 refers to ITGA2B, also known as Integrin alpha-IIb. Swiss-Prot accession number P08514 provides exemplary human CD41 amino acid sequences. In some embodiments, CD41 or CD41 molecule is a naturally-existing CD41 or a functional variant or fragment thereof.

P-selectin refers to SELP, also known as CD62P, GMP-140 or LECAM3. Swiss-Prot accession number P16109 provides exemplary human P-selectin amino acid sequences. In some embodiments, P-selectin or P-selectin molecule is a naturally-existing P-selectin or a functional variant or fragment thereof.

Clec2 refers to CLECIB, also known as C-type lectin domain family 1 member B. Swiss-Prot accession number Q9P126 provides exemplary human Clec2 amino acid sequences. In some embodiments, Clec2 or Clec2 molecule is a naturally-existing Clec2 or a functional variant or fragment thereof.

cKIT refers to mast/stem cell growth factor receptor kit, also known as CD117. Swiss-Prot accession number P10721 provides exemplary human cKIT amino acid sequences. In some embodiments, cKIT or cKIT molecule is a naturally-existing cKIT or a functional variant or fragment thereof.

FLT3 refers to receptor-type tyrosine-protein kinase FLT3, also known as CD135. Swiss-Prot accession number P36888 provides exemplary human FLT3 amino acid sequences. In some embodiments, FLT3 or FLT3 molecule is a naturally-existing FLT3 or a functional variant or fragment thereof.

MPL refers to thrombopoietin receptor, also known as C7403. Swiss-Prot accession number P40238 provides exemplary human MPL amino acid sequences. In some embodiments, MPL or MPL molecule is a naturally-existing MPL or a functional variant or fragment thereof.

ITGB3 refers to Integrin beta-3, also known as CD61. Swiss-Prot accession number P05106 provides exemplary human ITGB3 amino acid sequences. In some embodiments, ITGB3 or ITGB3 molecule is a naturally-existing ITGB3 or a functional variant or fragment thereof.

ITGB2 refers to Integrin beta-2, also known as CD18. Swiss-Prot accession number P05107 provides exemplary human ITGB2 amino acid sequences. In some embodiments, ITGB2 or ITGB2 molecule is a naturally-existing ITGB2 or a functional variant or fragment thereof.

GP5 refers to platelet glycoprotein V, also known as CD42d. Swiss-Prot accession number P40197 provides exemplary human GP5 amino acid sequences. In some embodiments, GP5 or GP5 molecule is a naturally-existing GP5 or a functional variant or fragment thereof.

GP6 refers to platelet glycoprotein VI. Swiss-Prot accession number Q9HCN6 provides exemplary human GP6 amino acid sequences. In some embodiments, GP6 or GP6 molecule is a naturally-existing GP6 or a functional variant or fragment thereof.

GP9 refers to platelet glycoprotein IX, also known as CD42a. Swiss-Prot accession number P14770 provides exemplary human GP9 amino acid sequences. In some embodiments, GP9 or GP9 molecule is a naturally-existing GP9 or a functional variant or fragment thereof.

GP1BA refers to platelet glycoprotein Ib alpha chain, also known as CD42b. Swiss-Prot accession number P07359 provides exemplary human GP1BA amino acid sequences. In some embodiments, GP1BA or GP1BA molecule is a naturally-existing GP1BA or a functional variant or fragment thereof.

DSC2 refers to desmocollin-2, also known as cadherin family member 2. Swiss-Prot accession number Q02487 provides exemplary human DSC2 amino acid sequences. In some embodiments, DSC2 or DSC2 molecule is a naturally-existing DSC2 or a functional variant or fragment thereof.

FCGR2A refers to Fc-gamma-RIIa, also known as CD32. Swiss-Prot accession number P12318 provides exemplary human FCGR2A amino acid sequences. In some embodiments, FCGR2A or FCGR2A molecule is a naturally-existing FCGR2A or a functional variant or fragment thereof.

TNFRSF10A refers to Tumor necrosis factor receptor superfamily member 10A, also known as Death receptor 4, TNF-related apoptosis-inducing ligand receptor 1, TRAIL-R1, or CD261. Swiss-Prot accession number 000220 provides exemplary human TNFRSF10A amino acid sequences. In some embodiments, TNFRSF10A or TNFRSF10A molecule is a naturally-existing TNFRSF10A or a functional variant or fragment thereof.

TNFRSF10B refers to Tumor necrosis factor receptor superfamily member 10B, also known as Death receptor 5, TNF-related apoptosis-inducing ligand receptor 2, TRAIL-R2, or CD262. Swiss-Prot accession number 014763 provides exemplary human TNFRSF10B amino acid sequences. In some embodiments, TNFRSF10B or TNFRSF10B molecule is a naturally-existing TNFRSF10B or a functional variant or fragment thereof.

TM4SF1 refers to transmembrane 4 L6 family member 1. Swiss-Prot accession number P30408 provides exemplary human TM4SF1 amino acid sequences. In some embodiments, TM4SF1 or TM4SF1 molecule is a naturally-existing TM4SF1 or a functional variant or fragment thereof.

In some embodiments, the multispecific and/or multifunctional molecule comprises one or more additional tumor-targeting moieties. In some embodiments, the one or more additional tumor-targeting moieties target (e.g., bind to) the same tumor antigen as the first tumor-targeting moiety. In some embodiments, the one or more additional tumor-targeting moieties target (e.g., bind to) a different tumor antigen from the first tumor-targeting moiety. In some embodiments, the multispecific and/or multifunctional molecule comprises a plurality of tumor-targeting moieties targeting different tumor antigens present on the same cell (e.g., a tumor cell). In some embodiments, the multispecific and/or multifunctional molecule comprises a plurality of tumor-targeting moieties targeting different tumor antigens present on different cells (e.g., 2, 3, 4, 5, 6, 7, 8, 9, 10, or more different tumor cells). In some embodiments, each of the tumor antigens is selected from: G6B, CD34, CD41, P-selectin, Clec2, cKIT, FLT3, MPL, ITGB3, ITGB2, GP5, GP6, GP9, GP1BA, DSC2, FCGR2A, TNFRSF10A, TNFRSF10B, or TM4SF1.

In some embodiments, the multispecific and/or multifunctional molecule comprises a first tumor-targeting moiety (e.g., targeting a first tumor antigen) and a second tumor-targeting moiety (e.g., targeting a second tumor antigen). In some embodiments, the first and second tumor antigens are present on the same tumor cell. In some embodiments, the first and third tumor antigens are present on the same tumor cell. In some embodiments, the second and third tumor antigens are present on the same tumor cell. In some embodiments, the first, second, and third tumor antigens are present on the same tumor cell. In some embodiments, the first and second tumor antigens are present on different tumor cells. In some embodiments, the first and third tumor antigens are present on different tumor cells. In some embodiments, the second and third tumor antigens are present on different tumor cells. In some embodiments, the first, second, and third tumor antigens are present on different tumor cells.

In some embodiments, the first, second, and/or third tumor antigens show higher expression in a tumor cell, e.g., a myeloproliferative neoplasm cell, than a non-tumor cell. In some embodiments, the expression of the first, second, and/or third tumor antigens in a tumor cell, e.g., a myeloproliferative neoplasm cell, is at least 1.5, 2, 4, 6, 8, or 10-fold higher than the expression of the first, second, and/or third tumor antigens in a non-tumor cell. In some embodiments, the multifunctional molecule preferentially binds to a tumor cell, e.g., a myeloproliferative neoplasm cell, over a non-tumor cell. In some embodiments, the binding between the multifunctional molecule and the tumor cell, e.g., a myeloproliferative neoplasm cell, is more than 10, 20, 30, 40, 50-fold greater than the binding between the multifunctional molecule and a non-tumor cell. In some embodiments, the affinity, e.g., the combined affinity, of the first and second tumor-targeting moieties for a tumor cell, e.g., a myeloproliferative neoplasm cell, is greater than the affinity of a similar multifunctional molecule having only one of the first tumor-targeting moiety or the second tumor-targeting moiety. In some embodiments, the affinity, e.g., the combined affinity, of the first and second tumor-targeting moieties for a tumor cell, e.g., a myeloproliferative neoplasm cell, is at least 2, 5, 10, 20, 30, 40, 50, 75 or 100 times greater than the affinity of a similar multifunctional molecule having only one of the first tumor-targeting moiety or the second tumor-targeting moiety.

In some embodiments, the affinity, e.g., the combined affinity, of the first, second, and third tumor-targeting moieties for a tumor cell, e.g., a myeloproliferative neoplasm cell, is greater than the affinity of a similar multifunctional molecule having only one of the first tumor-targeting moiety, the second tumor-targeting moiety, or the third tumor-targeting moiety, or a similar multifunctional molecule having only two of the first tumor-targeting moiety, the second tumor-targeting moiety, or the third tumor-targeting moiety. In some embodiments, the affinity, e.g., the combined affinity, of the first, second, and third tumor-targeting moieties for a tumor cell, e.g., a myeloproliferative neoplasm cell, is at least 2, 5, 10, 20, 30, 40, 50, 75 or 100 times greater than the affinity of a similar multifunctional molecule having only one of the first tumor-targeting moiety, the second tumor-targeting moiety, or the third tumor-targeting moiety, or a similar multifunctional molecule having only two of the first tumor-targeting moiety, the second tumor-targeting moiety, or the third tumor-targeting moiety.

In some embodiments, the affinity, e.g., the combined affinity, for the first and second tumor antigens of the first tumor-targeting moiety and the second tumor-targeting moiety is equal to or greater than the affinity of (iii), (iv) or (v), either alone or as part of the multifunctional molecule, for its corresponding binding member. In some embodiments, the affinity, e.g., the combined affinity, for the first and second tumor antigens of the first tumor-targeting moiety and the second tumor-targeting moiety is at least 2, 5, 10, 20, 30, 40, 50, 75 or 100 times greater than the affinity of (iii), (iv) or (v), either alone or as part of the multifunctional molecule, for its corresponding binding member.

In some embodiments, the affinity, e.g., the combined affinity, for the first, second, and third tumor antigens of the first tumor-targeting moiety, the second tumor-targeting moiety, and the third tumor-targeting moiety is equal to or greater than the affinity of (iii), (iv) or (v), either alone or as part of the multifunctional molecule, for its corresponding binding member. In some embodiments, the affinity, e.g., the combined affinity, for the first, second, and third tumor antigens of the first tumor-targeting moiety, the second tumor-targeting moiety, and the third tumor-targeting moiety is at least 2, 5, 10, 20, 30, 40, 50, 75 or 100 times greater than the affinity of (iii), (iv) or (v), either alone or as part of the multifunctional molecule, for its corresponding binding member.

In some embodiments of the aforementioned aspects, the first tumor antigen is CD34 and the second tumor antigen is CD41. In some embodiments, the first tumor antigen is CD34 and the second tumor antigen is G6B. In some embodiments, the first tumor antigen is CD41 and the second tumor antigen is G6B. In some embodiments, the first tumor antigen is CD34, the second tumor antigen is CD41, and the third tumor antigen is G6B.

In some embodiments of the aforementioned aspects, the first tumor antigen is P-selectin and the second tumor antigen is Clec2. In some embodiments, the first tumor antigen is CD34 and the second tumor antigen is P-selectin. In some embodiments, the first tumor antigen is CD41 and the second tumor antigen is P-selectin. In some embodiments, the first tumor antigen is G6B and the second tumor antigen is P-selectin. In some embodiments, the first tumor antigen is CD34 and the second tumor antigen is Clec2. In some embodiments, the first tumor antigen is CD41 and the second tumor antigen is Clec2. In some embodiments, the first tumor antigen is G6B and the second tumor antigen is Clec2. In some embodiments, the first tumor antigen is CD34, the second tumor antigen is CD41, and the third tumor antigen is P-selectin. In some embodiments, the first tumor antigen is CD34, the second tumor antigen is G6B, and the third tumor antigen is P-selectin. In some embodiments, the first tumor antigen is CD41, the second tumor antigen is G6B, and the third tumor antigen is P-selectin. In some embodiments, the first tumor antigen is CD34, the second tumor antigen is CD41, and the third tumor antigen is Clec2. In some embodiments, the first tumor antigen is CD34, the second tumor antigen is G6B, and the third tumor antigen is Clec2. In some embodiments, the first tumor antigen is CD41, the second tumor antigen is G6B, and the third tumor antigen is Clec2. In some embodiments, the first tumor antigen is CD34, the second tumor antigen is P-selectin, and the third tumor antigen is Clec2. In some embodiments, the first tumor antigen is CD41, the second tumor antigen is P-selectin, and the third tumor antigen is Clec2. In some embodiments, the first tumor antigen is G6B, the second tumor antigen is P-selectin, and the third tumor antigen is Clec2.

In some embodiments of the aforementioned aspects, the first tumor antigen is CD34 and the second tumor antigen is CD34. In some embodiments, the first tumor antigen is CD34 and the second tumor antigen is CD41. In some embodiments, the first tumor antigen is CD34 and the second tumor antigen is G6B. In some embodiments, the first tumor antigen is CD34 and the second tumor antigen is P-selectin. In some embodiments, the first tumor antigen is CD34 and the second tumor antigen is Clec2. In some embodiments, the first tumor antigen is CD34 and the second tumor antigen is cKIT. In some embodiments, the first tumor antigen is CD34 and the second tumor antigen is FLT3. In some embodiments, the first tumor antigen is CD34 and the second tumor antigen is MPL. In some embodiments, the first tumor antigen is CD34 and the second tumor antigen is ITGB3. In some embodiments, the first tumor antigen is CD34 and the second tumor antigen is ITGB2. In some embodiments, the first tumor antigen is CD34 and the second tumor antigen is GP5. In some embodiments, the first tumor antigen is CD34 and the second tumor antigen is GP6. In some embodiments, the first tumor antigen is CD34 and the second tumor antigen is GP9. In some embodiments, the first tumor antigen is CD34 and the second tumor antigen is GP1BA. In some embodiments, the first tumor antigen is CD34 and the second tumor antigen is DSC2. In some embodiments, the first tumor antigen is CD34 and the second tumor antigen is FCGR2A. In some embodiments, the first tumor antigen is CD34 and the second tumor antigen is TNFRSF10A. In some embodiments, the first tumor antigen is CD34 and the second tumor antigen is TNFRSF10B. In some embodiments, the first tumor antigen is CD34 and the second tumor antigen is TM4SF1.

In some embodiments of the aforementioned aspects, the first tumor antigen is CD41 and the second tumor antigen is CD34. In some embodiments, the first tumor antigen is CD41 and the second tumor antigen is CD41. In some embodiments, the first tumor antigen is CD41 and the second tumor antigen is G6B. In some embodiments, the first tumor antigen is CD41 and the second tumor antigen is P-selectin. In some embodiments, the first tumor antigen is CD41 and the second tumor antigen is Clec2. In some embodiments, the first tumor antigen is CD41 and the second tumor antigen is cKIT. In some embodiments, the first tumor antigen is CD41 and the second tumor antigen is FLT3. In some embodiments, the first tumor antigen is CD41 and the second tumor antigen is MPL. In some embodiments, the first tumor antigen is CD41 and the second tumor antigen is ITGB3. In some embodiments, the first tumor antigen is CD41 and the second tumor antigen is ITGB2. In some embodiments, the first tumor antigen is CD41 and the second tumor antigen is GP5. In some embodiments, the first tumor antigen is CD41 and the second tumor antigen is GP6. In some embodiments, the first tumor antigen is CD41 and the second tumor antigen is GP9. In some embodiments, the first tumor antigen is CD41 and the second tumor antigen is GP1BA. In some embodiments, the first tumor antigen is CD41 and the second tumor antigen is DSC2. In some embodiments, the first tumor antigen is CD41 and the second tumor antigen is FCGR2A. In some embodiments, the first tumor antigen is CD41 and the second tumor antigen is TNFRSF10A. In some embodiments, the first tumor antigen is CD41 and the second tumor antigen is TNFRSF10B. In some embodiments, the first tumor antigen is CD41 and the second tumor antigen is TM4SF1.

In some embodiments of the aforementioned aspects, the first tumor antigen is G6B and the second tumor antigen is CD34. In some embodiments, the first tumor antigen is G6B and the second tumor antigen is CD41. In some embodiments, the first tumor antigen is G6B and the second tumor antigen is G6B. In some embodiments, the first tumor antigen is G6B and the second tumor antigen is P-selectin. In some embodiments, the first tumor antigen is G6B and the second tumor antigen is Clec2. In some embodiments, the first tumor antigen is G6B and the second tumor antigen is cKIT. In some embodiments, the first tumor antigen is G6B and the second tumor antigen is FLT3. In some embodiments, the first tumor antigen is G6B and the second tumor antigen is MPL. In some embodiments, the first tumor antigen is G6B and the second tumor antigen is ITGB3. In some embodiments, the first tumor antigen is G6B and the second tumor antigen is ITGB2. In some embodiments, the first tumor antigen is G6B and the second tumor antigen is GP5. In some embodiments, the first tumor antigen is G6B and the second tumor antigen is GP6. In some embodiments, the first tumor antigen is G6B and the second tumor antigen is GP9. In some embodiments, the first tumor antigen is G6B and the second tumor antigen is GP1BA. In some embodiments, the first tumor antigen is G6B and the second tumor antigen is DSC2. In some embodiments, the first tumor antigen is G6B and the second tumor antigen is FCGR2A. In some embodiments, the first tumor antigen is G6B and the second tumor antigen is TNFRSF10A. In some embodiments, the first tumor antigen is G6B and the second tumor antigen is TNFRSF10B. In some embodiments, the first tumor antigen is G6B and the second tumor antigen is TM4SF1.

In some embodiments of the aforementioned aspects, the first tumor antigen is P-selectin and the second tumor antigen is CD34. In some embodiments, the first tumor antigen is P-selectin and the second tumor antigen is CD41. In some embodiments, the first tumor antigen is P-selectin and the second tumor antigen is G6B. In some embodiments, the first tumor antigen is P-selectin and the second tumor antigen is P-selectin. In some embodiments, the first tumor antigen is P-selectin and the second tumor antigen is Clec2. In some embodiments, the first tumor antigen is P-selectin and the second tumor antigen is cKIT. In some embodiments, the first tumor antigen is P-selectin and the second tumor antigen is FLT3. In some embodiments, the first tumor antigen is P-selectin and the second tumor antigen is MPL. In some embodiments, the first tumor antigen is P-selectin and the second tumor antigen is ITGB3. In some embodiments, the first tumor antigen is P-selectin and the second tumor antigen is ITGB2. In some embodiments, the first tumor antigen is P-selectin and the second tumor antigen is GP5. In some embodiments, the first tumor antigen is P-selectin and the second tumor antigen is GP6. In some embodiments, the first tumor antigen is P-selectin and the second tumor antigen is GP9. In some embodiments, the first tumor antigen is P-selectin and the second tumor antigen is GP1BA. In some embodiments, the first tumor antigen is P-selectin and the second tumor antigen is DSC2. In some embodiments, the first tumor antigen is P-selectin and the second tumor antigen is FCGR2A. In some embodiments, the first tumor antigen is P-selectin and the second tumor antigen is TNFRSF10A. In some embodiments, the first tumor antigen is P-selectin and the second tumor antigen is TNFRSF10B. In some embodiments, the first tumor antigen is P-selectin and the second tumor antigen is TM4SF1.

In some embodiments of the aforementioned aspects, the first tumor antigen is Clec2 and the second tumor antigen is CD34. In some embodiments, the first tumor antigen is Clec2 and the second tumor antigen is CD41. In some embodiments, the first tumor antigen is Clec2 and the second tumor antigen is G6B. In some embodiments, the first tumor antigen is Clec2 and the second tumor antigen is P-selectin. In some embodiments, the first tumor antigen is Clec2 and the second tumor antigen is Clec2. In some embodiments, the first tumor antigen is Clec2 and the second tumor antigen is cKIT. In some embodiments, the first tumor antigen is Clec2 and the second tumor antigen is FLT3. In some embodiments, the first tumor antigen is Clec2 and the second tumor antigen is MPL. In some embodiments, the first tumor antigen is Clec2 and the second tumor antigen is ITGB3. In some embodiments, the first tumor antigen is Clec2 and the second tumor antigen is ITGB2. In some embodiments, the first tumor antigen is Clec2 and the second tumor antigen is GP5. In some embodiments, the first tumor antigen is Clec2 and the second tumor antigen is GP6. In some embodiments, the first tumor antigen is Clec2 and the second tumor antigen is GP9. In some embodiments, the first tumor antigen is Clec2 and the second tumor antigen is GP1BA. In some embodiments, the first tumor antigen is Clec2 and the second tumor antigen is DSC2. In some embodiments, the first tumor antigen is Clec2 and the second tumor antigen is FCGR2A. In some embodiments, the first tumor antigen is Clec2 and the second tumor antigen is TNFRSF10A. In some embodiments, the first tumor antigen is Clec2 and the second tumor antigen is TNFRSF10B. In some embodiments, the first tumor antigen is Clec2 and the second tumor antigen is TM4SF1.

In some embodiments of the aforementioned aspects, the first tumor antigen is cKIT and the second tumor antigen is CD34. In some embodiments, the first tumor antigen is cKIT and the second tumor antigen is CD41. In some embodiments, the first tumor antigen is cKIT and the second tumor antigen is G6B. In some embodiments, the first tumor antigen is cKIT and the second tumor antigen is P-selectin. In some embodiments, the first tumor antigen is cKIT and the second tumor antigen is Clec2. In some embodiments, the first tumor antigen is cKIT and the second tumor antigen is cKIT. In some embodiments, the first tumor antigen is cKIT and the second tumor antigen is FLT3. In some embodiments, the first tumor antigen is cKIT and the second tumor antigen is MPL. In some embodiments, the first tumor antigen is cKIT and the second tumor antigen is ITGB3. In some embodiments, the first tumor antigen is cKIT and the second tumor antigen is ITGB2. In some embodiments, the first tumor antigen is cKIT and the second tumor antigen is GP5. In some embodiments, the first tumor antigen is cKIT and the second tumor antigen is GP6. In some embodiments, the first tumor antigen is cKIT and the second tumor antigen is GP9. In some embodiments, the first tumor antigen is cKIT and the second tumor antigen is GP1BA. In some embodiments, the first tumor antigen is cKIT and the second tumor antigen is DSC2. In some embodiments, the first tumor antigen is cKIT and the second tumor antigen is FCGR2A. In some embodiments, the first tumor antigen is cKIT and the second tumor antigen is TNFRSF10A. In some embodiments, the first tumor antigen is cKIT and the second tumor antigen is TNFRSF10B. In some embodiments, the first tumor antigen is cKIT and the second tumor antigen is TM4SF1.

In some embodiments of the aforementioned aspects, the first tumor antigen is FLT3 and the second tumor antigen is CD34. In some embodiments, the first tumor antigen is FLT3 and the second tumor antigen is CD41. In some embodiments, the first tumor antigen is FLT3 and the second tumor antigen is G6B. In some embodiments, the first tumor antigen is FLT3 and the second tumor antigen is P-selectin. In some embodiments, the first tumor antigen is FLT3 and the second tumor antigen is Clec2. In some embodiments, the first tumor antigen is FLT3 and the second tumor antigen is cKIT. In some embodiments, the first tumor antigen is FLT3 and the second tumor antigen is FLT3. In some embodiments, the first tumor antigen is FLT3 and the second tumor antigen is MPL. In some embodiments, the first tumor antigen is FLT3 and the second tumor antigen is ITGB3. In some embodiments, the first tumor antigen is FLT3 and the second tumor antigen is ITGB2. In some embodiments, the first tumor antigen is FLT3 and the second tumor antigen is GP5. In some embodiments, the first tumor antigen is FLT3 and the second tumor antigen is GP6. In some embodiments, the first tumor antigen is FLT3 and the second tumor antigen is GP9. In some embodiments, the first tumor antigen is FLT3 and the second tumor antigen is GP1BA. In some embodiments, the first tumor antigen is FLT3 and the second tumor antigen is DSC2. In some embodiments, the first tumor antigen is FLT3 and the second tumor antigen is FCGR2A. In some embodiments, the first tumor antigen is FLT3 and the second tumor antigen is TNFRSF10A. In some embodiments, the first tumor antigen is FLT3 and the second tumor antigen is TNFRSF10B. In some embodiments, the first tumor antigen is FLT3 and the second tumor antigen is TM4SF1.

In some embodiments of the aforementioned aspects, the first tumor antigen is MPL and the second tumor antigen is CD34. In some embodiments, the first tumor antigen is MPL and the second tumor antigen is CD41. In some embodiments, the first tumor antigen is MPL and the second tumor antigen is G6B. In some embodiments, the first tumor antigen is MPL and the second tumor antigen is P-selectin. In some embodiments, the first tumor antigen is MPL and the second tumor antigen is Clec2. In some embodiments, the first tumor antigen is MPL and the second tumor antigen is cKIT. In some embodiments, the first tumor antigen is MPL and the second tumor antigen is FLT3. In some embodiments, the first tumor antigen is MPL and the second tumor antigen is MPL. In some embodiments, the first tumor antigen is MPL and the second tumor antigen is ITGB3. In some embodiments, the first tumor antigen is MPL and the second tumor antigen is ITGB2. In some embodiments, the first tumor antigen is MPL and the second tumor antigen is GP5. In some embodiments, the first tumor antigen is MPL and the second tumor antigen is GP6. In some embodiments, the first tumor antigen is MPL and the second tumor antigen is GP9. In some embodiments, the first tumor antigen is MPL and the second tumor antigen is GP1BA. In some embodiments, the first tumor antigen is MPL and the second tumor antigen is DSC2. In some embodiments, the first tumor antigen is MPL and the second tumor antigen is FCGR2A. In some embodiments, the first tumor antigen is MPL and the second tumor antigen is TNFRSF10A. In some embodiments, the first tumor antigen is MPL and the second tumor antigen is TNFRSF10B. In some embodiments, the first tumor antigen is MPL and the second tumor antigen is TM4SF1.

In some embodiments of the aforementioned aspects, the first tumor antigen is ITGB3 and the second tumor antigen is CD34. In some embodiments, the first tumor antigen is ITGB3 and the second tumor antigen is CD41. In some embodiments, the first tumor antigen is ITGB3 and the second tumor antigen is G6B. In some embodiments, the first tumor antigen is ITGB3 and the second tumor antigen is P-selectin. In some embodiments, the first tumor antigen is ITGB3 and the second tumor antigen is Clec2. In some embodiments, the first tumor antigen is ITGB3 and the second tumor antigen is cKIT. In some embodiments, the first tumor antigen is ITGB3 and the second tumor antigen is FLT3. In some embodiments, the first tumor antigen is ITGB3 and the second tumor antigen is MPL. In some embodiments, the first tumor antigen is ITGB3 and the second tumor antigen is ITGB3. In some embodiments, the first tumor antigen is ITGB3 and the second tumor antigen is ITGB2. In some embodiments, the first tumor antigen is ITGB3 and the second tumor antigen is GP5. In some embodiments, the first tumor antigen is ITGB3 and the second tumor antigen is GP6. In some embodiments, the first tumor antigen is ITGB3 and the second tumor antigen is GP9. In some embodiments, the first tumor antigen is ITGB3 and the second tumor antigen is GP1BA. In some embodiments, the first tumor antigen is ITGB3 and the second tumor antigen is DSC2. In some embodiments, the first tumor antigen is ITGB3 and the second tumor antigen is FCGR2A. In some embodiments, the first tumor antigen is ITGB3 and the second tumor antigen is TNFRSF10A. In some embodiments, the first tumor antigen is ITGB3 and the second tumor antigen is TNFRSF10B. In some embodiments, the first tumor antigen is ITGB3 and the second tumor antigen is TM4SF1.

In some embodiments of the aforementioned aspects, the first tumor antigen is ITGB2 and the second tumor antigen is CD34. In some embodiments, the first tumor antigen is ITGB2 and the second tumor antigen is CD41. In some embodiments, the first tumor antigen is ITGB2 and the second tumor antigen is G6B. In some embodiments, the first tumor antigen is ITGB2 and the second tumor antigen is P-selectin. In some embodiments, the first tumor antigen is ITGB2 and the second tumor antigen is Clec2. In some embodiments, the first tumor antigen is ITGB2 and the second tumor antigen is cKIT. In some embodiments, the first tumor antigen is ITGB2 and the second tumor antigen is FLT3. In some embodiments, the first tumor antigen is ITGB2 and the second tumor antigen is MPL. In some embodiments, the first tumor antigen is ITGB2 and the second tumor antigen is ITGB3. In some embodiments, the first tumor antigen is ITGB2 and the second tumor antigen is ITGB2. In some embodiments, the first tumor antigen is ITGB2 and the second tumor antigen is GP5. In some embodiments, the first tumor antigen is ITGB2 and the second tumor antigen is GP6. In some embodiments, the first tumor antigen is ITGB2 and the second tumor antigen is GP9. In some embodiments, the first tumor antigen is ITGB2 and the second tumor antigen is GP1BA. In some embodiments, the first tumor antigen is ITGB2 and the second tumor antigen is DSC2. In some embodiments, the first tumor antigen is ITGB2 and the second tumor antigen is FCGR2A. In some embodiments, the first tumor antigen is ITGB2 and the second tumor antigen is TNFRSF10A. In some embodiments, the first tumor antigen is ITGB2 and the second tumor antigen is TNFRSF10B. In some embodiments, the first tumor antigen is ITGB2 and the second tumor antigen is TM4SF1.

In some embodiments of the aforementioned aspects, the first tumor antigen is GP5 and the second tumor antigen is CD34. In some embodiments, the first tumor antigen is GP5 and the second tumor antigen is CD41. In some embodiments, the first tumor antigen is GP5 and the second tumor antigen is G6B. In some embodiments, the first tumor antigen is GP5 and the second tumor antigen is P-selectin. In some embodiments, the first tumor antigen is GP5 and the second tumor antigen is Clec2. In some embodiments, the first tumor antigen is GP5 and the second tumor antigen is cKIT. In some embodiments, the first tumor antigen is GP5 and the second tumor antigen is FLT3. In some embodiments, the first tumor antigen is GP5 and the second tumor antigen is MPL. In some embodiments, the first tumor antigen is GP5 and the second tumor antigen is ITGB3. In some embodiments, the first tumor antigen is GP5 and the second tumor antigen is ITGB2. In some embodiments, the first tumor antigen is GP5 and the second tumor antigen is GP5. In some embodiments, the first tumor antigen is GP5 and the second tumor antigen is GP6. In some embodiments, the first tumor antigen is GP5 and the second tumor antigen is GP9. In some embodiments, the first tumor antigen is GP5 and the second tumor antigen is GP1BA. In some embodiments, the first tumor antigen is GP5 and the second tumor antigen is DSC2. In some embodiments, the first tumor antigen is GP5 and the second tumor antigen is FCGR2A. In some embodiments, the first tumor antigen is GP5 and the second tumor antigen is TNFRSF10A. In some embodiments, the first tumor antigen is GP5 and the second tumor antigen is TNFRSF10B. In some embodiments, the first tumor antigen is GP5 and the second tumor antigen is TM4SF1.

In some embodiments of the aforementioned aspects, the first tumor antigen is GP6 and the second tumor antigen is CD34. In some embodiments, the first tumor antigen is GP6 and the second tumor antigen is CD41. In some embodiments, the first tumor antigen is GP6 and the second tumor antigen is G6B. In some embodiments, the first tumor antigen is GP6 and the second tumor antigen is P-selectin. In some embodiments, the first tumor antigen is GP6 and the second tumor antigen is Clec2. In some embodiments, the first tumor antigen is GP6 and the second tumor antigen is cKIT. In some embodiments, the first tumor antigen is GP6 and the second tumor antigen is FLT3. In some embodiments, the first tumor antigen is GP6 and the second tumor antigen is MPL. In some embodiments, the first tumor antigen is GP6 and the second tumor antigen is ITGB3. In some embodiments, the first tumor antigen is GP6 and the second tumor antigen is ITGB2. In some embodiments, the first tumor antigen is GP6 and the second tumor antigen is GP5. In some embodiments, the first tumor antigen is GP6 and the second tumor antigen is GP6. In some embodiments, the first tumor antigen is GP6 and the second tumor antigen is GP9. In some embodiments, the first tumor antigen is GP6 and the second tumor antigen is GP1BA. In some embodiments, the first tumor antigen is GP6 and the second tumor antigen is DSC2. In some embodiments, the first tumor antigen is GP6 and the second tumor antigen is FCGR2A. In some embodiments, the first tumor antigen is GP6 and the second tumor antigen is TNFRSF10A. In some embodiments, the first tumor antigen is GP6 and the second tumor antigen is TNFRSF10B. In some embodiments, the first tumor antigen is GP6 and the second tumor antigen is TM4SF1.

In some embodiments of the aforementioned aspects, the first tumor antigen is GP9 and the second tumor antigen is CD34. In some embodiments, the first tumor antigen is GP9 and the second tumor antigen is CD41. In some embodiments, the first tumor antigen is GP9 and the second tumor antigen is G6B. In some embodiments, the first tumor antigen is GP9 and the second tumor antigen is P-selectin. In some embodiments, the first tumor antigen is GP9 and the second tumor antigen is Clec2. In some embodiments, the first tumor antigen is GP9 and the second tumor antigen is cKIT. In some embodiments, the first tumor antigen is GP9 and the second tumor antigen is FLT3. In some embodiments, the first tumor antigen is GP9 and the second tumor antigen is MPL. In some embodiments, the first tumor antigen is GP9 and the second tumor antigen is ITGB3. In some embodiments, the first tumor antigen is GP9 and the second tumor antigen is ITGB2. In some embodiments, the first tumor antigen is GP9 and the second tumor antigen is GP5. In some embodiments, the first tumor antigen is GP9 and the second tumor antigen is GP6. In some embodiments, the first tumor antigen is GP9 and the second tumor antigen is GP9. In some embodiments, the first tumor antigen is GP9 and the second tumor antigen is GP1BA. In some embodiments, the first tumor antigen is GP9 and the second tumor antigen is DSC2. In some embodiments, the first tumor antigen is GP9 and the second tumor antigen is FCGR2A. In some embodiments, the first tumor antigen is GP9 and the second tumor antigen is TNFRSF10A. In some embodiments, the first tumor antigen is GP9 and the second tumor antigen is TNFRSF10B. In some embodiments, the first tumor antigen is GP9 and the second tumor antigen is TM4SF1.

In some embodiments of the aforementioned aspects, the first tumor antigen is GP1BA and the second tumor antigen is CD34. In some embodiments, the first tumor antigen is GP1BA and the second tumor antigen is CD41. In some embodiments, the first tumor antigen is GP1BA and the second tumor antigen is G6B. In some embodiments, the first tumor antigen is GP1BA and the second tumor antigen is P-selectin. In some embodiments, the first tumor antigen is GP1BA and the second tumor antigen is Clec2. In some embodiments, the first tumor antigen is GP1BA and the second tumor antigen is cKIT. In some embodiments, the first tumor antigen is GP1BA and the second tumor antigen is FLT3. In some embodiments, the first tumor antigen is GP1BA and the second tumor antigen is MPL. In some embodiments, the first tumor antigen is GP1BA and the second tumor antigen is ITGB3. In some embodiments, the first tumor antigen is GP1BA and the second tumor antigen is ITGB2. In some embodiments, the first tumor antigen is GP1BA and the second tumor antigen is GP5. In some embodiments, the first tumor antigen is GP1BA and the second tumor antigen is GP6. In some embodiments, the first tumor antigen is GP1BA and the second tumor antigen is GP9. In some embodiments, the first tumor antigen is GP1BA and the second tumor antigen is GP1BA. In some embodiments, the first tumor antigen is GP1BA and the second tumor antigen is DSC2. In some embodiments, the first tumor antigen is GP1BA and the second tumor antigen is FCGR2A. In some embodiments, the first tumor antigen is GP1BA and the second tumor antigen is TNFRSF10A. In some embodiments, the first tumor antigen is GP1BA and the second tumor antigen is TNFRSF10B. In some embodiments, the first tumor antigen is GP1BA and the second tumor antigen is TM4SF1.

In some embodiments of the aforementioned aspects, the first tumor antigen is DSC2 and the second tumor antigen is CD34. In some embodiments, the first tumor antigen is DSC2 and the second tumor antigen is CD41. In some embodiments, the first tumor antigen is DSC2 and the second tumor antigen is G6B. In some embodiments, the first tumor antigen is DSC2 and the second tumor antigen is P-selectin. In some embodiments, the first tumor antigen is DSC2 and the second tumor antigen is Clec2. In some embodiments, the first tumor antigen is DSC2 and the second tumor antigen is cKIT. In some embodiments, the first tumor antigen is DSC2 and the second tumor antigen is FLT3. In some embodiments, the first tumor antigen is DSC2 and the second tumor antigen is MPL. In some embodiments, the first tumor antigen is DSC2 and the second tumor antigen is ITGB3. In some embodiments, the first tumor antigen is DSC2 and the second tumor antigen is ITGB2. In some embodiments, the first tumor antigen is DSC2 and the second tumor antigen is GP5. In some embodiments, the first tumor antigen is DSC2 and the second tumor antigen is GP6. In some embodiments, the first tumor antigen is DSC2 and the second tumor antigen is GP9. In some embodiments, the first tumor antigen is DSC2 and the second tumor antigen is GP1BA. In some embodiments, the first tumor antigen is DSC2 and the second tumor antigen is DSC2. In some embodiments, the first tumor antigen is DSC2 and the second tumor antigen is FCGR2A. In some embodiments, the first tumor antigen is DSC2 and the second tumor antigen is TNFRSF10A. In some embodiments, the first tumor antigen is DSC2 and the second tumor antigen is TNFRSF10B. In some embodiments, the first tumor antigen is DSC2 and the second tumor antigen is TM4SF1.

In some embodiments of the aforementioned aspects, the first tumor antigen is FCGR2A and the second tumor antigen is CD34. In some embodiments, the first tumor antigen is FCGR2A and the second tumor antigen is CD41. In some embodiments, the first tumor antigen is FCGR2A and the second tumor antigen is G6B. In some embodiments, the first tumor antigen is FCGR2A and the second tumor antigen is P-selectin. In some embodiments, the first tumor antigen is FCGR2A and the second tumor antigen is Clec2. In some embodiments, the first tumor antigen is FCGR2A and the second tumor antigen is cKIT. In some embodiments, the first tumor antigen is FCGR2A and the second tumor antigen is FLT3. In some embodiments, the first tumor antigen is FCGR2A and the second tumor antigen is MPL. In some embodiments, the first tumor antigen is FCGR2A and the second tumor antigen is ITGB3. In some embodiments, the first tumor antigen is FCGR2A and the second tumor antigen is ITGB2. In some embodiments, the first tumor antigen is FCGR2A and the second tumor antigen is GP5. In some embodiments, the first tumor antigen is FCGR2A and the second tumor antigen is GP6. In some embodiments, the first tumor antigen is FCGR2A and the second tumor antigen is GP9. In some embodiments, the first tumor antigen is FCGR2A and the second tumor antigen is GP1BA. In some embodiments, the first tumor antigen is FCGR2A and the second tumor antigen is DSC2. In some embodiments, the first tumor antigen is FCGR2A and the second tumor antigen is FCGR2A. In some embodiments, the first tumor antigen is FCGR2A and the second tumor antigen is TNFRSF10A. In some embodiments, the first tumor antigen is FCGR2A and the second tumor antigen is TNFRSF10B. In some embodiments, the first tumor antigen is FCGR2A and the second tumor antigen is TM4SF1.

In some embodiments of the aforementioned aspects, the first tumor antigen is TNFRSF10A and the second tumor antigen is CD34. In some embodiments, the first tumor antigen is TNFRSF10A and the second tumor antigen is CD41. In some embodiments, the first tumor antigen is TNFRSF10A and the second tumor antigen is G6B. In some embodiments, the first tumor antigen is TNFRSF10A and the second tumor antigen is P-selectin. In some embodiments, the first tumor antigen is TNFRSF10A and the second tumor antigen is Clec2. In some embodiments, the first tumor antigen is TNFRSF10A and the second tumor antigen is cKIT. In some embodiments, the first tumor antigen is TNFRSF10A and the second tumor antigen is FLT3. In some embodiments, the first tumor antigen is TNFRSF10A and the second tumor antigen is MPL. In some embodiments, the first tumor antigen is TNFRSF10A and the second tumor antigen is ITGB3. In some embodiments, the first tumor antigen is TNFRSF10A and the second tumor antigen is ITGB2. In some embodiments, the first tumor antigen is TNFRSF10A and the second tumor antigen is GP5. In some embodiments, the first tumor antigen is TNFRSF10A and the second tumor antigen is GP6. In some embodiments, the first tumor antigen is TNFRSF10A and the second tumor antigen is GP9. In some embodiments, the first tumor antigen is TNFRSF10A and the second tumor antigen is GP1BA. In some embodiments, the first tumor antigen is TNFRSF10A and the second tumor antigen is DSC2. In some embodiments, the first tumor antigen is TNFRSF10A and the second tumor antigen is FCGR2A. In some embodiments, the first tumor antigen is TNFRSF10A and the second tumor antigen is TNFRSF10A. In some embodiments, the first tumor antigen is TNFRSF10A and the second tumor antigen is TM4SF1.

In some embodiments of the aforementioned aspects, the first tumor antigen is TNFRSF10B and the second tumor antigen is CD34. In some embodiments, the first tumor antigen is TNFRSF10B and the second tumor antigen is CD41. In some embodiments, the first tumor antigen is TNFRSF10B and the second tumor antigen is G6B. In some embodiments, the first tumor antigen is TNFRSF10B and the second tumor antigen is P-selectin. In some embodiments, the first tumor antigen is TNFRSF10B and the second tumor antigen is Clec2. In some embodiments, the first tumor antigen is TNFRSF10B and the second tumor antigen is cKIT. In some embodiments, the first tumor antigen is TNFRSF10B and the second tumor antigen is FLT3. In some embodiments, the first tumor antigen is TNFRSF10B and the second tumor antigen is MPL. In some embodiments, the first tumor antigen is TNFRSF10B and the second tumor antigen is ITGB3. In some embodiments, the first tumor antigen is TNFRSF10B and the second tumor antigen is ITGB2. In some embodiments, the first tumor antigen is TNFRSF10B and the second tumor antigen is GP5. In some embodiments, the first tumor antigen is TNFRSF10B and the second tumor antigen is GP6. In some embodiments, the first tumor antigen is TNFRSF10B and the second tumor antigen is GP9. In some embodiments, the first tumor antigen is TNFRSF10B and the second tumor antigen is GP1BA. In some embodiments, the first tumor antigen is TNFRSF10B and the second tumor antigen is DSC2. In some embodiments, the first tumor antigen is TNFRSF10B and the second tumor antigen is FCGR2A. In some embodiments, the first tumor antigen is TNFRSF10B and the second tumor antigen is TNFRSF10B. In some embodiments, the first tumor antigen is TNFRSF10B and the second tumor antigen is TM4SF1.

In some embodiments of the aforementioned aspects, the first tumor antigen is TM4SF1 and the second tumor antigen is CD34. In some embodiments, the first tumor antigen is TM4SF1 and the second tumor antigen is CD41. In some embodiments, the first tumor antigen is TM4SF1 and the second tumor antigen is G6B. In some embodiments, the first tumor antigen is TM4SF1 and the second tumor antigen is P-selectin. In some embodiments, the first tumor antigen is TM4SF1 and the second tumor antigen is Clec2. In some embodiments, the first tumor antigen is TM4SF1 and the second tumor antigen is cKIT. In some embodiments, the first tumor antigen is TM4SF1 and the second tumor antigen is FLT3. In some embodiments, the first tumor antigen is TM4SF1 and the second tumor antigen is MPL. In some embodiments, the first tumor antigen is TM4SF1 and the second tumor antigen is ITGB3. In some embodiments, the first tumor antigen is TM4SF1 and the second tumor antigen is ITGB2. In some embodiments, the first tumor antigen is TM4SF1 and the second tumor antigen is GP5. In some embodiments, the first tumor antigen is TM4SF1 and the second tumor antigen is GP6. In some embodiments, the first tumor antigen is TM4SF1 and the second tumor antigen is GP9. In some embodiments, the first tumor antigen is TM4SF1 and the second tumor antigen is GP1BA. In some embodiments, the first tumor antigen is TM4SF1 and the second tumor antigen is DSC2. In some embodiments, the first tumor antigen is TM4SF1 and the second tumor antigen is FCGR2A. In some embodiments, the first tumor antigen is TM4SF1 and the second tumor antigen is TNFRSF10A. In some embodiments, the first tumor antigen is TM4SF1 and the second tumor antigen is TNFRSF10B. In some embodiments, the first tumor antigen is TM4SF1 and the second tumor antigen is TM4SF1.

### Antibody Molecules Targeting Tumor Antigens

In some embodiments, the tumor-targeting moiety comprises a CDR, a framework region, or a variable region sequence shown in Table 38 (or a sequence having at least about 75%, 80%, 85%, 90%, 95%, or 99% sequence identity thereto).

TABLE 38
Sequences for exemplary antibodies capable of binding to exemplary target molecules

| Target | Description | SEQ ID NO | Sequence |
|---|---|---|---|
| CD34 | Exemplary | SEQ ID | EVQLQQSGPELVKPGASVKISCKASGYSFIGYFMNWV |
| | anti-CD34 | NO: 2001 | MQSHGRSLEWIGRINPYNGYTFYNQKFKGKATLTVD |
| | VH | | KSSSTAHMELRSLASEDSAVYYCARHFRYDGVFYYA |
| | | | MDYWGQGTSVTVSS |
| | Exemplary | SEQ ID | QLVLTQSSSASFSLGASAKLTCTLSSQHSTFTIEWYQQ |
| | anti-CD34 | NO: 2002 | QPLKPPKYVMDLKKDGSHSTGDGVPDRFSGSSSGAD |
| | VL | | RYLSISNIQPEDEATYICGVGDTIKEQFVYVFGGGTKV |
| | | | TVL |
| cKIT | Exemplary | SEQ ID | EVQLVESGGGLVQPGGSLRLSCAASGFAFSGYYMAW |
| (CD117) | anti-cKIT | NO: 2003 | VRQAPGKGLEWVANINYPGSSTYYLDSVKGRFTISRD |
| | VH | | NAKNSLYLQMNSLRAEDTAVYYCARGDYYGTTYWY |
| | | | FDVWGQGTTVTVSS |
| | Exemplary | SEQ ID | DIQMTQSPSSLSASVGDRVTITCRASQSISSYLNWYQQ |
| | anti-cKIT | NO: 2004 | KPGKAPKLLIYYTSRLQSGVPSRFSGSGSGTDFTLTISS |
| | VL | | LQPEDFATYYCQQGRRLWSFGGGTKVEIK |
| FLT3 | Exemplary | SEQ ID | QVQLQQPGAELVKPGASLKLSCKSSGYTFTSYWMHW |
| | anti-FLT3 | NO: 2005 | VRQRPGHGLEWIGEIDPSDSYKDYNQKFKDKATLTVD |
| | VH | | RSSNTAYMHLSSLTSDDSAVYYCARAITTTPFDFWGQ |
| | | | GTTLTVSS |
| | Exemplary | SEQ ID | DIVLTQSPATLSVTPGDSVSLSCRASQSISNNLHWYQQ |
| | anti-FLT3 | NO: 2006 | KSHESPRLLIKYASQSISGIPSRFSGSGSGTDFTLSINSV |
| | VL | | ETEDFGVYFCQQSNTWPYTFGGGTKLEIKR |
| CD41 | Exemplary | SEQ ID | EVQLQQSGAELVKPGASVKLSCTASGFNIKDTYVHW |
| (ITGA2B) | anti-CD41 | NO: 2007 | VKQRPEQGLEWIGRIDPANGYTKYDPKFQGKATITAD |
| | VH | | TSSNTAYLQLSSLTSEDTAVYYCVRPLYDYYAMDYW |
| | | | GQGTSVTVSS |
| | Exemplary | SEQ ID | DILMTQSPSSMSVSLGDTVSITCHASQGISSNIGWLQQ |
| | anti-CD41 | NO: 2008 | KPGKSFMGLIYYGTNLVDGVPSRFSGSGSGADYSLTIS |
| | VL | | SLDSEDFADYYCVQYAQLPYTFGGGTKLEIK |
| MPL | 1.75 VH | SEQ ID | EVLVESGGGLVQPKGSLKLSCAASGFSFNTYAMNW |
| | | NO: 2009 | VRQAPGKGLEWIAHIRSKSNNFATYYADSVKDRFSIS |
| | | | RDASENILFLQMNNLKTEDTAMYYCVRQGGDFPMDY |
| | | | WGQGTSVTVSS |
| | 1.75 VL | SEQ ID | QIVLTQSPAIMSASPGEKVTISCSASSSVSYMYWYQQK |
| | | NO: 2010 | PGSSPKPWIYRTSNLASGVPARFSGSGSGTSYSLTISN |
| | | | MEAEDAAAYYCQQYHSYPTTFGGGTKLEVK |
| | 1.78 VH | SEQ ID | QVQLQQSGPELVKPGASVKMSCKASGYAFSSSWLNW |
| | | NO: 2011 | VRQRPGKGLEWIGRIYPGDGENHYNGKFKGKATLTA |
| | | | DKSSSTGYMQLSSLTSEDSAVYFCASYYEGGYWGQG |
| | | | TLITVSA |
| | 1.78 VL | SEQ ID | DIVMTQAAPSIPVTPGESVSISCRSDKSLLHSNGNTYLF |
| | | NO: 2012 | WFLQRPGQSPQLLIYRMSNLASGVPDRFSGSGSGTAF |
| | | | TLRISGVEAEDVGVYYCMQHLEYPYTFGGGTKLEIK |
| P- | Exemplary | SEQ ID | EVLVESGGGLVRPGGSLRLSCAASGFTFSNYDMHW |
| Selectin | anti-P- | NO: 2013 | VRQATGKGLEWVSAITAAGDIYYPGSVKGRFTISREN |
| (SELP) | Selectin VH | | AKNSLYLQMNSLRAGDTAVYYCARGRYSGSGSYYN |
| | | | DWFDPWGQGTLVTVSS |
| | Exemplary | SEQ ID | EIVLTQSPATLSLSPGERATLSCRASQSVSSYLAWYQQ |
| | anti-P- | NO: 2014 | KPGQAPRLLIYDASNRATGIPARFSGSGSGTDFTLTISS |
| | Selectin VL | | LEPEDFAVYYCQQRSNWPLTFGGGTKVEIK |
| DSC2 | Exemplary | SEQ ID | MDSRLNLVFLVLILKGVQCDVQLVESGGGLVQPGGS |
| | anti-DSC2 | NO: 2015 | RKLSCAASGFTFSSFGMHWVRQAPEKGLEWVAYISSG |
| | #1 VH | | SSTIYYADTVKGRFTISRDNPKNTLFLQMTSLRSEDTA |
| | | | MYYCARVHYYYFDYWGQGTTLTVSS |
| | Exemplary | SEQ ID | MRPSIQFLGLLLFWLHGAQCDIQMTQSPSSLSASLGGK |
| | anti-DSC2 | NO: 2016 | VTITCKASQDINKYIAWYQHKPGKGPRLLIHYTSTLQP |
| | #1 VL | | GIPSRFSGSGSGRDYSFSISNLEPEDIATYYCLQYDNLW |
| | | | TFGGGTKL |
| | Exemplary | SEQ ID | MAWVWTLLFLMAAAQSIQAQIQLVQSGPELKKPGET |
| | anti-DSC2 | NO: 2017 | VKISCKASGYTFTDYSMHWVKQAPGKGLKWMGWIN |
| | #2 VH | | TETGEPTYADDFKGRFAFSLETSASTAYLQINNLKNED |
| | | | TATYFCARWLLFDYWGQGTTLTVSS |
| | Exemplary | SEQ ID | MESQTQVLMFLLLWVSGACADIVMTQSPSSLAMSVG |
| | anti-DSC2 | NO: 2018 | QKVTMSCKSSQSLLNSSNQKNYLAWYQQKPGQSPKL |
| | #2 VL | | LVYFASTRESGVPDRFIGSGSGTDFTLTISSVQAEDLA |
| | | | DYFCQQHYSTPLTFGAGTKL |
| FCGR2A | AT-10 VH | SEQ ID | EVKLEESGGGLVQPGGSMKLSCVASGFTFSYYWMN |
| (CD32a) | | NO: 2019 | WVRQSPEKGLEWVAEIRLKSNNYATHYAESVKGRFTI |
| | | | SRDDSKNNVYLQMNNLRAEDTGIYYCNRRDEYYAM |
| | | | DYWGQGTSVSVSS |
| | AT-10 VL | SEQ ID | DIVLTQSPGSLAVSLGQRATISCRASESVDNFGISFMN |
| | | NO: 2020 | WFQQKPGQPPRLLIYGASNQGSGVPARFSGSGSGTDF |
| | | | SLNIHPVEEDDAAMYFCQQSKEVPWTFGGGTKLEIK |
| | IV.3 VH | SEQ ID | QIQLVQSGPELKKPGETVKISCKASGYTFTNYGMNWV |
| | | NO: 2021 | KQAPGKGLKWMGWLNTYTGESIYPDDFKGRFAFSSE |
| | | | TSASTAYLQINNLKNEDMATYFCARGDYGYDDPLDY |
| | | | WGQGTSVTVSS |
| | IV.3 VL | SEQ ID | DIVMTQAAPSVPVTPGESVSISCRSSKSLLHTNGNTYL |
| | | NO: 2022 | HWFLQRPGQSPQLLIYRMSVLASGVPDRFSGSGSGTA |
| | | | FTLSISRVEAEDVGVFYCMQHLEYPLTFGAGTKLELK |
| | MDE-8 VH | SEQ ID | QVHLVESGGGVVQPGRSLRLSCAASGFTFSSYGMHW |
| | | NO: 2023 | VRQAPGKGLEWVAVIWYDGSNYYYTDSVKGRFTISR |
| | | | DNSKNTLYLQMNSLRAEDTAVYYCARDLGAAASDY |
| | | | WGQGTLVTVSS |
| | MDE-8 VL | SEQ ID | AIQLTQSPSSLSASVGDRVTITCRASQGINSALAWYQQ |
| | | NO: 2024 | KPGKAPKLLIYDASSLESGVPSRFSGSGSGTDFTLTISS |
| | | | LQPEDFATYYCQQFNSYPHTFGQGTKLEIK |
| TNFRS | E-11-13 VH | SEQ ID | MDLMCKKMKHLWFFLLLVAAPRWVLSQLQLQESGP |
| F10A | | NO: 2025 | GLVKPSETLSLTCTVSGGSIISKSSYWGWIRQPPGKGL |
| or | | | EWIGSIYYSGSTFYNPSLKSRVTISVDTSKNQFSLKLSS |
| TNFRS | | | VTAADTAVYYCARLTVAEFDYWGQGTLVTVSSAS |
| F10B | E-11-13 VL | SEQ ID | MEAPAQLLFLLLLWLPDTTGEIVLTQSPATLSLSPGER |
| | | NO: 2026 | ATLSCRASQSVSSFLAWYQQKPGQAPRLLIYDASNRA |
| | | | TGIPARFSGSGSGTDFTLTISSLEPEDFAVYYCQQRSN |
| | | | WPLTFGPGTKVDIKRT |
| | L-30-10 VH | SEQ ID | MDLMCKKMKHLWFFLLLVAAPRWVLSQLQLQESGP |
| | | NO: 2027 | GLVKPSETLSLTCTVSGGSISSRSNYWGWIRQPPGKGL |
| | | | EWIGNVYYRGSTYYNSSLKSRVTISVDTSKNQFSLKLS |
| | | | SVTVADTAVYYCARLSVAEFDYWGQGILVTVSSAS |
| | L-30-10 VL | SEQ ID | MEAPAQLLFLLLLWLPDTTGEIVLTQSPATLSLSPGER |
| | | NO: 2028 | ATLSCRASQSVSSFLAWYQQKPGQAPRLLIYDASNRA |
| | | | TGSPARFSGSGSGTDFTLTISSLEPEDFAVYYCQQRSD |
| | | | WPLTFGPGTKVDIKRT |
| | H-48-2 VH | SEQ ID | MDLMCKKMKHLWFFLLLVAAPRWVLSQLQLQESGP |
| | | NO: 2029 | GLVKPSETLSLTCTVSGGSISSSSYYWGWVRQPPGKG |
| | | | LEWIGSIHYSGSTFYNPSLKSRVTISVDTSKNQFSLKLS |
| | | | SVTAADTTVYYCARQGSTVVRGVYYYGMDVWGQG |
| | | | TTVTVSSAS |
| | H-48-2 VL | SEQ ID | METPAQLLFLLLLWLPDTTGEIVLTQSPGTLSLSPGER |
| | | NO: 2030 | ATLSCRASQSVSSSYLAWYQQKPGQAPRLLIYGASSR |
| | | | ATGIPDRFSGSGSGTDFTLTISRLEPEDFAVYYCQQYG |
| | | | SSPLYTFGQGTKLEIKRT |
| | 0304 VH | SEQ ID | MDWTWRILFLVAAATSAHSQVQLVQSGAEMKKPGA |
| | | NO: 2031 | SVKVSCKTSGYTFTNYKINWVRQAPGQGLEWMGWM |
| | | | NPDTDSTGYPQKFQGRVTMTRNTSISTAYMELSSLRS |
| | | | EDTAVYYCARSYGSGSYYRDYYYGMDVWGQGTTVT |
| | | | VSS |
| | 0304 VL | SEQ ID | MEAPAQLLFLLLLWLPDTTGEIVLTQSPATLSLSPGER |
| | | NO: 2032 | ATLSCRASQSVSSYLAWYQQKPGQAPRLLIYDASNRA |
| | | | TGIPARFSGSGSGTDFTLTISSLEPEDFAVYYCQQRSN |
| | | | WPLTFGGGTKVEIKR |
| | KMTR1 VH | SEQ ID | MEFGLSWLFLVAILKGVQCEVQLLESGGGLVQPGRSL |
| | | NO: 2033 | RLSCAASGFTFSSYAMSWVRQAPGKGLEWVSAISGSG |
| | | | GSRYYADSVKGRFTISRDNSKNTLYLQMNSLRAEDTA |
| | | | VYYCAKESSGWFGAFDYWGQGTLVTVSS |
| | KMTRI VL | SEQ ID | MSPSQLIGFLLLWVPASRGEIVLTQSPDFQSVTPKEKV |
| | | NO: 2034 | TITCRASQSIGSSLHWYQQKPDQSPKLLIKYASQSFSG |
| | | | VPSRFSGSGSGTDFTLTINSLEAEDAAAYYCHQSSSLPI |
| | | | TFGQGTRLEIKR |
| TM4SF1 | Exemplary | SEQ ID | EVILVESGGGLVKPGGSLKLSCAASGFTFSSFAMSWV |
| | anti- | NO: 2035 | RQTPEKRLEWVATISSGSIYIYYTDGVKGRFTISRDNA |
| | TM4SF1 | | KNTVHLQMSSLRSEDTAMYYCARRGIYYGYDGYAM |
| | VH | | DYWGQGTSVTVSS |
| | Exemplary | SEQ ID | AVVMTQTPLSLPVSLGDQASISCRSSQSLVHSNGNTYL |
| | anti- | NO: 2036 | HWYMQKPGQSPKVLIYKVSNRFSGVPDRFSGSGSGTD |
| | TM4SF1 | | FTLKISRVEADDLGIYFCSQSTHIPLAFGAGTKLELK |
| | VL | | |

In some embodiments, the first, second, or third tumor antigen is CD34. In some embodiments, the first, second, or third tumor-targeting moiety comprises:
(i) a HCDR1, HCDR2, and/or HCDR3 from SEQ ID NO: 2001 (or a sequence with no more than 1, 2, 3, or 4 mutations, e.g., substitutions, additions, or deletions),
(ii) a VH of SEQ ID NO: 2001 (or a sequence having at least about 75%, 80%, 85%, 90%, 95%, or 99% sequence identity thereto),
(iii) a LCDR1, LCDR2, and/or LCDR3 from SEQ ID NO: 2002 (or a sequence with no more than 1, 2, 3, or 4 mutations, e.g., substitutions, additions, or deletions), and/or
(iv) a VL of SEQ ID NO: 2002 (or a sequence having at least about 75%, 80%, 85%, 90%, 95%, or 99% sequence identity thereto).

In some embodiments, wherein the first, second, or third tumor antigen is CD41. In some embodiments, the first, second, or third tumor-targeting moiety comprises:
(i) a HCDR1, HCDR2, and/or HCDR3 from SEQ ID NO: 2007 (or a sequence with no more than 1, 2, 3, or 4 mutations, e.g., substitutions, additions, or deletions),
(ii) a VH of SEQ ID NO: 2007 (or a sequence having at least about 75%, 80%, 85%, 90%, 95%, or 99% sequence identity thereto),
(iii) a LCDR1, LCDR2, and/or LCDR3 from SEQ ID NO: 2008 (or a sequence with no more than 1, 2, 3, or 4 mutations, e.g., substitutions, additions, or deletions), and/or
(iv) a VL of SEQ ID NO: 2008 (or a sequence having at least about 75%, 80%, 85%, 90%, 95%, or 99% sequence identity thereto).

In some embodiments, the first, second, or third tumor antigen is P-selectin. In some embodiments, the first, second, or third tumor-targeting moiety comprises:
(i) a HCDR1, HCDR2, and/or HCDR3 from SEQ ID NO: 2013 (or a sequence with no more than 1, 2, 3, or 4 mutations, e.g., substitutions, additions, or deletions),
(ii) a VH of SEQ ID NO: 2013 (or a sequence having at least about 75%, 80%, 85%, 90%, 95%, or 99% sequence identity thereto),
(iii) a LCDR1, LCDR2, and/or LCDR3 from SEQ ID NO: 2014 (or a sequence with no more than 1, 2, 3, or 4 mutations, e.g., substitutions, additions, or deletions), and/or
(iv) a VL of SEQ ID NO: 2014 (or a sequence having at least about 75%, 80%, 85%, 90%, 95%, or 99% sequence identity thereto).

In some embodiments, the first, second, or third tumor antigen is cKIT. In some embodiments, the first, second, or third tumor-targeting moiety comprises:
(i) a HCDR1, HCDR2, and/or HCDR3 from SEQ ID NO: 2003 (or a sequence with no more than 1, 2, 3, or 4 mutations, e.g., substitutions, additions, or deletions),
(ii) a VH of SEQ ID NO: 2003 (or a sequence having at least about 75%, 80%, 85%, 90%, 95%, or 99% sequence identity thereto),
(iii) a LCDR1, LCDR2, and/or LCDR3 from SEQ ID NO: 2004 (or a sequence with no more than 1, 2, 3, or 4 mutations, e.g., substitutions, additions, or deletions), and/or
(iv) a VL of SEQ ID NO: 2004 (or a sequence having at least about 75%, 80%, 85%, 90%, 95%, or 99% sequence identity thereto).

In some embodiments, the first, second, or third tumor antigen is FLT3. In some embodiments, the first, second, or third tumor-targeting moiety comprises:
(i) a HCDR1, HCDR2, and/or HCDR3 from SEQ ID NO: 2005 (or a sequence with no more than 1, 2, 3, or 4 mutations, e.g., substitutions, additions, or deletions),
(ii) a VH of SEQ ID NO: 2005 (or a sequence having at least about 75%, 80%, 85%, 90%, 95%, or 99% sequence identity thereto),
(iii) a LCDR1, LCDR2, and/or LCDR3 from SEQ ID NO: 2006 (or a sequence with no more than 1, 2, 3, or 4 mutations, e.g., substitutions, additions, or deletions), and/or
(iv) a VL of SEQ ID NO: 2006 (or a sequence having at least about 75%, 80%, 85%, 90%, 95%, or 99% sequence identity thereto).

In some embodiments, the first, second, or third tumor antigen is MPL. In some embodiments, the first, second, or third tumor-targeting moiety comprises:
(i) (a) a HCDR1, HCDR2, and/or HCDR3 from SEQ ID NO: 2009 (or a sequence with no more than 1, 2, 3, or 4 mutations, e.g., substitutions, additions, or deletions),
(b) a VH of SEQ ID NO: 2009 (or a sequence having at least about 75%, 80%, 85%, 90%, 95%, or 99% sequence identity thereto),
(c) a LCDR1, LCDR2, and/or LCDR3 from SEQ ID NO: 2010 (or a sequence with no more than 1, 2, 3, or 4 mutations, e.g., substitutions, additions, or deletions), and/or
(d) a VL of SEQ ID NO: 2010 (or a sequence having at least about 75%, 80%, 85%, 90%, 95%, or 99% sequence identity thereto); or
(ii) (a) a HCDR1, HCDR2, and/or HCDR3 from SEQ ID NO: 2011 (or a sequence with no more than 1, 2, 3, or 4 mutations, e.g., substitutions, additions, or deletions),
(b) a VH of SEQ ID NO: 2011 (or a sequence having at least about 75%, 80%, 85%, 90%, 95%, or 99% sequence identity thereto),
(c) a LCDR1, LCDR2, and/or LCDR3 from SEQ ID NO: 2012 (or a sequence with no more than 1, 2, 3, or 4 mutations, e.g., substitutions, additions, or deletions), and/or
(d) a VL of SEQ ID NO: 2012 (or a sequence having at least about 75%, 80%, 85%, 90%, 95%, or 99% sequence identity thereto).

In some embodiments, the first, second, or third tumor antigen is DSC2. In some embodiments, the first, second, or third tumor-targeting moiety comprises:
(i) (a) a HCDR1, HCDR2, and/or HCDR3 from SEQ ID NO: 2015 (or a sequence with no more than 1, 2, 3, or 4 mutations, e.g., substitutions, additions, or deletions),
(b) a VH of SEQ ID NO: 2015 (or a sequence having at least about 75%, 80%, 85%, 90%, 95%, or 99% sequence identity thereto),
(c) a LCDR1, LCDR2, and/or LCDR3 from SEQ ID NO: 2016 (or a sequence with no more than 1, 2, 3, or 4 mutations, e.g., substitutions, additions, or deletions), and/or
(d) a VL of SEQ ID NO: 2016 (or a sequence having at least about 75%, 80%, 85%, 90%, 95%, or 99% sequence identity thereto); or
(ii) (a) a HCDR1, HCDR2, and/or HCDR3 from SEQ ID NO: 2017 (or a sequence with no more than 1, 2, 3, or 4 mutations, e.g., substitutions, additions, or deletions),
(b) a VH of SEQ ID NO: 2017 (or a sequence having at least about 75%, 80%, 85%, 90%, 95%, or 99% sequence identity thereto),
(c) a LCDR1, LCDR2, and/or LCDR3 from SEQ ID NO: 2018 (or a sequence with no more than 1, 2, 3, or 4 mutations, e.g., substitutions, additions, or deletions), and/or
(d) a VL of SEQ ID NO: 2018 (or a sequence having at least about 75%, 80%, 85%, 90%, 95%, or 99% sequence identity thereto).

In some embodiments, the first, second, or third tumor antigen is FCGR2A. In some embodiments, the first, second, or third tumor-targeting moiety comprises:
(i) (a) a HCDR1, HCDR2, and/or HCDR3 from SEQ ID NO: 2019 (or a sequence with no more than 1, 2, 3, or 4 mutations, e.g., substitutions, additions, or deletions),
(b) a VH of SEQ ID NO: 2019 (or a sequence having at least about 75%, 80%, 85%, 90%, 95%, or 99% sequence identity thereto),
(c) a LCDR1, LCDR2, and/or LCDR3 from SEQ ID NO: 2020 (or a sequence with no more than 1, 2, 3, or 4 mutations, e.g., substitutions, additions, or deletions), and/or
(d) a VL of SEQ ID NO: 2020 (or a sequence having at least about 75%, 80%, 85%, 90%, 95%, or 99% sequence identity thereto);
(ii) (a) a HCDR1, HCDR2, and/or HCDR3 from SEQ ID NO: 2021 (or a sequence with no more than 1, 2, 3, or 4 mutations, e.g., substitutions, additions, or deletions),
(b) a VH of SEQ ID NO:20 21 (or a sequence having at least about 75%, 80%, 85%, 90%, 95%, or 99% sequence identity thereto),
(c) a LCDR1, LCDR2, and/or LCDR3 from SEQ ID NO: 2022 (or a sequence with no more than 1, 2, 3, or 4 mutations, e.g., substitutions, additions, or deletions), and/or
(d) a VL of SEQ ID NO: 2022 (or a sequence having at least about 75%, 80%, 85%, 90%, 95%, or 99% sequence identity thereto), or
(iii) (a) a HCDR1, HCDR2, and/or HCDR3 from SEQ ID NO: 2023 (or a sequence with no more than 1, 2, 3, or 4 mutations, e.g., substitutions, additions, or deletions),
(b) a VH of SEQ ID NO: 2023 (or a sequence having at least about 75%, 80%, 85%, 90%, 95%, or 99% sequence identity thereto),
(c) a LCDR1, LCDR2, and/or LCDR3 from SEQ ID NO: 2024 (or a sequence with no more than 1, 2, 3, or 4 mutations, e.g., substitutions, additions, or deletions), and/or
(d) a VL of SEQ ID NO: 2024 (or a sequence having at least about 75%, 80%, 85%, 90%, 95%, or 99% sequence identity thereto).

In some embodiments, the first, second, or third tumor antigen is TNFRSF10A or TNFRSF10B. In some embodiments, the first, second, or third tumor-targeting moiety comprises:
(i) (a) a HCDR1, HCDR2, and/or HCDR3 from SEQ ID NO: 2025 (or a sequence with no more than 1, 2, 3, or 4 mutations, e.g., substitutions, additions, or deletions),
(b) a VH of SEQ ID NO: 2025 (or a sequence having at least about 75%, 80%, 85%, 90%, 95%, or 99% sequence identity thereto),
(c) a LCDR1, LCDR2, and/or LCDR3 from SEQ ID NO: 2026 (or a sequence with no more than 1, 2, 3, or 4 mutations, e.g., substitutions, additions, or deletions), and/or
(d) a VL of SEQ ID NO: 2026 (or a sequence having at least about 75%, 80%, 85%, 90%, 95%, or 99% sequence identity thereto); or
(ii) (a) a HCDR1, HCDR2, and/or HCDR3 from SEQ ID NO: 2027 (or a sequence with no more than 1, 2, 3, or 4 mutations, e.g., substitutions, additions, or deletions),
(b) a VH of SEQ ID NO: 2027 (or a sequence having at least about 75%, 80%, 85%, 90%, 95%, or 99% sequence identity thereto),
(c) a LCDR1, LCDR2, and/or LCDR3 from SEQ ID NO: 2028 (or a sequence with no more than 1, 2, 3, or 4 mutations, e.g., substitutions, additions, or deletions), and/or
(d) a VL of SEQ ID NO: 2028 (or a sequence having at least about 75%, 80%, 85%, 90%, 95%, or 99% sequence identity thereto);
(iii) (a) a HCDR1, HCDR2, and/or HCDR3 from SEQ ID NO: 2029 (or a sequence with no more than 1, 2, 3, or 4 mutations, e.g., substitutions, additions, or deletions),
(b) a VH of SEQ ID NO: 2029 (or a sequence having at least about 75%, 80%, 85%, 90%, 95%, or 99% sequence identity thereto),
(c) a LCDR1, LCDR2, and/or LCDR3 from SEQ ID NO: 2030 (or a sequence with no more than 1, 2, 3, or 4 mutations, e.g., substitutions, additions, or deletions), and/or
(d) a VL of SEQ ID NO: 2030 (or a sequence having at least about 75%, 80%, 85%, 90%, 95%, or 99% sequence identity thereto);
(iv) (a) a HCDR1, HCDR2, and/or HCDR3 from SEQ ID NO: 2031 (or a sequence with no more than 1, 2, 3, or 4 mutations, e.g., substitutions, additions, or deletions),
(b) a VH of SEQ ID NO: 2031 (or a sequence having at least about 75%, 80%, 85%, 90%, 95%, or 99% sequence identity thereto),
(c) a LCDR1, LCDR2, and/or LCDR3 from SEQ ID NO: 2032 (or a sequence with no more than 1, 2, 3, or 4 mutations, e.g., substitutions, additions, or deletions), and/or
(d) a VL of SEQ ID NO: 2032 (or a sequence having at least about 75%, 80%, 85%, 90%, 95%, or 99% sequence identity thereto); or
(v) (a) a HCDR1, HCDR2, and/or HCDR3 from SEQ ID NO: 2033 (or a sequence with no more than 1, 2, 3, or 4 mutations, e.g., substitutions, additions, or deletions),
(b) a VH of SEQ ID NO: 2033 (or a sequence having at least about 75%, 80%, 85%, 90%, 95%, or 99% sequence identity thereto),
(c) a LCDR1, LCDR2, and/or LCDR3 from SEQ ID NO: 2034 (or a sequence with no more than 1, 2, 3, or 4 mutations, e.g., substitutions, additions, or deletions), and/or
(d) a VL of SEQ ID NO: 2034 (or a sequence having at least about 75%, 80%, 85%, 90%, 95%, or 99% sequence identity thereto).

In some embodiments, the first, second, or third tumor antigen is TM4SF1. In some embodiments, the first, second, or third tumor-targeting moiety comprises:
(i) a HCDR1, HCDR2, and/or HCDR3 from SEQ ID NO: 2035 (or a sequence with no more than 1, 2, 3, or 4 mutations, e.g., substitutions, additions, or deletions),
(ii) a VH of SEQ ID NO: 2035 (or a sequence having at least about 75%, 80%, 85%, 90%, 95%, or 99% sequence identity thereto),
(iii) a LCDR1, LCDR2, and/or LCDR3 from SEQ ID NO: 2036 (or a sequence with no more than 1, 2, 3, or 4 mutations, e.g., substitutions, additions, or deletions), and/or
(iv) a VL of SEQ ID NO: 2036 (or a sequence having at least about 75%, 80%, 85%, 90%, 95%, or 99% sequence identity thereto).

### Exemplary Anti-CD34 Antibody Sequences

In one aspect, provided herein is a multispecific or multifunctional molecule comprising a tumor targeting moiety that comprises a CD34-targeting moiety. In another aspect, provided herein is an anti-CD34 antibody molecule (e.g., a monoclonal anti-CD34 antibody molecule).

In some embodiments, the CD34-targeting moiety or anti-CD34 antibody molecule comprises an antibody, or an antigen-binding fragment thereof, disclosed in Table 20 or Table 21. In some embodiments, the CD34-targeting moiety or anti-CD34 antibody molecule comprises a CDR, a framework region, or a variable region sequence disclosed in Table 20 or Table 21 (or a sequence having at least about 75%, 80%, 85%, 90%, 95%, or 99% sequence identity thereto).

In some embodiments, the CD34-targeting moiety or anti-CD34 antibody molecule comprises a VH comprising a heavy chain complementarity determining region 1 (VHCDR1) amino acid sequence of SEQ ID NO: 6239 (or a sequence with no more than 1, 2, 3, or 4 mutations, e.g., substitutions, additions, or deletions), a VHCDR2 amino acid sequence of SEQ ID NO: 6241 (or a sequence with no more than 1, 2, 3, or 4 mutations, e.g., substitutions, additions, or deletions), and/or a VHCDR3 amino acid sequence of SEQ ID NO: 6243 (or a sequence with no more than 1, 2, 3, or 4 mutations, e.g., substitutions, additions, or deletions). In some embodiments, the CD34-targeting moiety or anti-CD34 antibody molecule comprises a VH comprising a VHCDR1 amino acid sequence of SEQ ID NO: 6239, a VHCDR2 amino acid sequence of SEQ ID NO: 6241, and/or a VHCDR3 amino acid sequence of SEQ ID NO: 6243. In some embodiments, the CD34-targeting moiety or anti-CD34 antibody molecule comprises a VL comprising a light chain complementarity determining region 1 (VLCDR1) amino acid sequence of SEQ ID NO: 6245 (or a sequence with no more than 1, 2, 3, or 4 mutations, e.g., substitutions, additions, or deletions), a VLCDR2 amino acid sequence of SEQ ID NO: 1236 (or a sequence with no more than 1, 2, 3, or 4 mutations, e.g., substitutions, additions, or deletions), and/or a VLCDR3 amino acid sequence of SEQ ID NO: 6246 (or a sequence with no more than 1, 2, 3, or 4 mutations, e.g., substitutions, additions, or deletions). In some embodiments, the CD34-targeting moiety or anti-CD34 antibody molecule comprises a VL comprising a VLCDR1 amino acid sequence of SEQ ID NO: 6245, a VLCDR2 amino acid sequence of SEQ ID NO: 1236, and a VLCDR3 amino acid sequence of SEQ ID NO: 6246.

In some embodiments, the CD34-targeting moiety or anti-CD34 antibody molecule comprises a VH comprising the amino acid sequence of SEQ ID NO: 79, 6225, 6227, or 6229, or an amino acid sequence having at least about 80%, 85%, 90%, 95%, or 99% sequence identity thereto. In some embodiments, the CD34-targeting moiety or anti-CD34 antibody molecule comprises a VL comprising the amino acid sequence of SEQ ID NO: 6231, 6233, 6235, or 6237, or an amino acid sequence having at least about 80%, 85%, 90%, 95%, or 99% sequence identity thereto. In some embodiments, the CD34-targeting moiety or anti-CD34 antibody molecule comprises a VH comprising the amino acid sequence of SEQ ID NO: 79 (or an amino acid sequence having at least about 80%, 85%, 90%, 95%, or 99% sequence identity thereto) and a VL comprising the amino acid sequence of SEQ ID NO: 6231 (or an amino acid sequence having at least about 80%, 85%, 90%, 95%, or 99% sequence identity thereto). In some embodiments, the CD34-targeting moiety or anti-CD34 antibody molecule comprises a VH comprising the amino acid sequence of SEQ ID NO: 79 and a VL comprising the amino acid sequence of SEQ ID NO: 6231. In some embodiments, the CD34-targeting moiety or anti-CD34 antibody molecule comprises a VH comprising the amino acid sequence of SEQ ID NO: 6225 (or an amino acid sequence having at least about 80%, 85%, 90%, 95%, or 99% sequence identity thereto) and a VL comprising the amino acid sequence of SEQ ID NO: 6231 (or an amino acid sequence having at least about 80%, 85%, 90%, 95%, or 99% sequence identity thereto). In some embodiments, the CD34-targeting moiety or anti-CD34 antibody molecule comprises a VH comprising the amino acid sequence of SEQ ID NO: 6225 and a VL comprising the amino acid sequence of SEQ ID NO: 6231. In some embodiments, the CD34-targeting moiety or anti-CD34 antibody molecule comprises a VH comprising the amino acid sequence of SEQ ID NO: 6227 (or an amino acid sequence having at least about 80%, 85%, 90%, 95%, or 99% sequence identity thereto) and a VL comprising the amino acid sequence of SEQ ID NO: 6231 (or an amino acid sequence having at least about 80%, 85%, 90%, 95%, or 99% sequence identity thereto). In some embodiments, the CD34-targeting moiety or anti-CD34 antibody molecule comprises a VH comprising the amino acid sequence of SEQ ID NO: 6227 and a VL comprising the amino acid sequence of SEQ ID NO: 6231. In some embodiments, the CD34-targeting moiety or anti-CD34 antibody molecule comprises a VH comprising the amino acid sequence of SEQ ID NO: 6229 (or an amino acid sequence having at least about 80%, 85%, 90%, 95%, or 99% sequence identity thereto) and a VL comprising the amino acid sequence of SEQ ID NO: 6231 (or an amino acid sequence having at least about 80%, 85%, 90%, 95%, or 99% sequence identity thereto). In some embodiments, the CD34-targeting moiety or anti-CD34 antibody molecule comprises a VH comprising the amino acid sequence of SEQ ID NO: 6229 and a VL comprising the amino acid sequence of SEQ ID NO: 6231. In some embodiments, the CD34-targeting moiety or anti-CD34 antibody molecule comprises a VH comprising the amino acid sequence of SEQ ID NO: 79 (or an amino acid sequence having at least about 80%, 85%, 90%, 95%, or 99% sequence identity thereto) and a VL comprising the amino acid sequence of SEQ ID NO: 6233 (or an amino acid sequence having at least about 80%, 85%, 90%, 95%, or 99% sequence identity thereto). In some embodiments, the CD34-targeting moiety or anti-CD34 antibody molecule comprises a VH comprising the amino acid sequence of SEQ ID NO: 79 and a VL comprising the amino acid sequence of SEQ ID NO: 6233. In some embodiments, the CD34-targeting moiety or anti-CD34 antibody molecule comprises a VH comprising the amino acid sequence of SEQ ID NO: 6225 (or an amino acid sequence having at least about 80%, 85%, 90%, 95%, or 99% sequence identity thereto) and a VL comprising the amino acid sequence of SEQ ID NO: 6233 (or an amino acid sequence having at least about 80%, 85%, 90%, 95%, or 99% sequence identity thereto). In some embodiments, the CD34-targeting moiety or anti-CD34 antibody molecule comprises a VH comprising the amino acid sequence of SEQ ID NO: 6225 and a VL comprising the amino acid sequence of SEQ ID NO: 6233. In some embodiments, the CD34-targeting moiety or anti-CD34 antibody molecule comprises a VH comprising the amino acid sequence of SEQ ID NO: 6227 (or an amino acid sequence having at least about 80%, 85%, 90%, 95%, or 99% sequence identity thereto) and a VL comprising the amino acid sequence of SEQ ID NO: 6233 (or an amino acid sequence having at least about 80%, 85%, 90%, 95%, or 99% sequence identity thereto). In some embodiments, the CD34-targeting moiety or anti-CD34 antibody molecule comprises a VH comprising the amino acid sequence of SEQ ID NO: 6227 and a VL comprising the amino acid sequence of SEQ ID NO: 6233. In some embodiments, the CD34-targeting moiety or anti-CD34 antibody molecule comprises a VH comprising the amino acid sequence of SEQ ID NO: 6229 (or an amino acid sequence having at least about 80%, 85%, 90%, 95%, or 99% sequence identity thereto) and a VL comprising the amino acid sequence of SEQ ID NO: 6233 (or an amino acid sequence having at least about 80%, 85%, 90%, 95%, or 99% sequence identity thereto). In some embodiments, the CD34-targeting moiety or anti-CD34 antibody molecule comprises a VH comprising the amino acid sequence of SEQ ID NO: 6229 and a VL comprising the amino acid sequence of SEQ ID NO: 6233. In some embodiments, the CD34-targeting moiety or anti-CD34 antibody molecule comprises a VH comprising the amino acid sequence of SEQ ID NO: 79 (or an amino acid sequence having at least about 80%, 85%, 90%, 95%, or 99% sequence identity thereto) and a VL comprising the amino acid sequence of SEQ ID NO: 6235 (or an amino acid sequence having at least about 80%, 85%, 90%, 95%, or 99% sequence identity thereto). In some embodiments, the CD34-targeting moiety or anti-CD34 antibody molecule comprises a VH comprising the amino acid sequence of SEQ ID NO: 79 and a VL comprising the amino acid sequence of SEQ ID NO: 6235. In some embodiments, the CD34-targeting moiety or anti-CD34 antibody molecule comprises a VH comprising the amino acid sequence of SEQ ID NO: 6225 (or an amino acid sequence having at least about 80%, 85%, 90%, 95%, or 99% sequence identity thereto) and a VL comprising the amino acid sequence of SEQ ID NO: 6235 (or an amino acid sequence having at least about 80%, 85%, 90%, 95%, or 99% sequence identity thereto). In some embodiments, the CD34-targeting moiety or anti-CD34 antibody molecule comprises a VH comprising the amino acid sequence of SEQ ID NO: 6225 and a VL comprising the amino acid sequence of SEQ ID NO: 6235. In some embodiments, the CD34-targeting moiety or anti-CD34 antibody molecule comprises a VH comprising the amino acid sequence of SEQ ID NO: 6227 (or an amino acid sequence having at least about 80%, 85%, 90%, 95%, or 99% sequence identity thereto) and a VL comprising the amino acid sequence of SEQ ID NO: 6235 (or an amino acid sequence having at least about 80%, 85%, 90%, 95%, or 99% sequence identity thereto). In some embodiments, the CD34-targeting moiety or anti-CD34 antibody molecule comprises a VH comprising the amino acid sequence of SEQ ID NO: 6227 and a VL comprising the amino acid sequence of SEQ ID NO: 6235. In some embodiments, the CD34-targeting moiety or anti-CD34 antibody molecule comprises a VH comprising the amino acid sequence of SEQ ID NO: 6229 (or an amino acid sequence having at least about 80%, 85%, 90%, 95%, or 99% sequence identity thereto) and a VL comprising the amino acid sequence of SEQ ID NO: 6235 (or an amino acid sequence having at least about 80%, 85%, 90%, 95%, or 99% sequence identity thereto). In some embodiments, the CD34-targeting moiety or anti-CD34 antibody molecule comprises a VH comprising the amino acid sequence of SEQ ID NO: 6229 and a VL comprising the amino acid sequence of SEQ ID NO: 6235. In some embodiments, the CD34-targeting moiety or anti-CD34 antibody molecule comprises a VH comprising the amino acid sequence of SEQ ID NO: 79 (or an amino acid sequence having at least about 80%, 85%, 90%, 95%, or 99% sequence identity thereto) and a VL comprising the amino acid sequence of SEQ ID NO: 6237 (or an amino acid sequence having at least about 80%, 85%, 90%, 95%, or 99% sequence identity thereto). In some embodiments, the CD34-targeting moiety or anti-CD34 antibody molecule comprises a VH comprising the amino acid sequence of SEQ ID NO: 79 and a VL comprising the amino acid sequence of SEQ ID NO: 6237. In some embodiments, the CD34-targeting moiety or anti-CD34 antibody molecule comprises a VH comprising the amino acid sequence of SEQ ID NO: 6225 (or an amino acid sequence having at least about 80%, 85%, 90%, 95%, or 99% sequence identity thereto) and a VL comprising the amino acid sequence of SEQ ID NO: 6237 (or an amino acid sequence having at least about 80%, 85%, 90%, 95%, or 99% sequence identity thereto). In some embodiments, the CD34-targeting moiety or anti-CD34 antibody molecule comprises a VH comprising the amino acid sequence of SEQ ID NO: 6225 and a VL comprising the amino acid sequence of SEQ ID NO: 6237. In some embodiments, the CD34-targeting moiety or anti-CD34 antibody molecule comprises a VH comprising the amino acid sequence of SEQ ID NO: 6227 (or an amino acid sequence having at least about 80%, 85%, 90%, 95%, or 99% sequence identity thereto) and a VL comprising the amino acid sequence of SEQ ID NO: 6237 (or an amino acid sequence having at least about 80%, 85%, 90%, 95%, or 99% sequence identity thereto). In some embodiments, the CD34-targeting moiety or anti-CD34 antibody molecule comprises a VH comprising the amino acid sequence of SEQ ID NO: 6227 and a VL comprising the amino acid sequence of SEQ ID NO: 6237. In some embodiments, the CD34-targeting moiety or anti-CD34 antibody molecule comprises a VH comprising the amino acid sequence of SEQ ID NO: 6229 (or an amino acid sequence having at least about 80%, 85%, 90%, 95%, or 99% sequence identity thereto) and a VL comprising the amino acid sequence of SEQ ID NO: 6237 (or an amino acid sequence having at least about 80%, 85%, 90%, 95%, or 99% sequence identity thereto). In some embodiments, the CD34-targeting moiety or anti-CD34 antibody molecule comprises a VH comprising the amino acid sequence of SEQ ID NO: 6229 and a VL comprising the amino acid sequence of SEQ ID NO: 6237.

In some embodiments, the CD34-targeting moiety or anti-CD34 antibody molecule comprises a VH comprising the amino acid sequence of SEQ ID NO: 79 and a VL comprising the amino acid sequence of SEQ ID NO: 6233.

TABLE 20
Exemplary variable region sequences of anti-CD34 antibodies

| SEQ ID | | |
|---|---|---|
| NO | Description | Sequence |
| SEQ ID | Mouse VH | EIQLQQSGPELMKPGASLKISCKTSGYSFTSYYMHWVKQSHGQ |
| NO: 6222 | | SLEWIGFIDPFKVITGYNHNFRGKATLTVDRSSTTAYMHLRSLT |
| | | SEDSAVYYCARRYYSDYDGYALDYWGQGTSVTVSS |
| SEQ ID | Mouse VL | DVVMTQTPLSLPVSLGDQASIFCRSSQSLVHSDGNTYLHWYLQ |
| NO: 6223 | | KPGQSPKLLIYKVSNRFSGVPDRFSGSGSGTDFTLKISRVEAEDL |
| | | GVYFCSQSTHVPPYTFGGGTKLEIK |
| SEQ ID | Humanization | QIQLQESGPGLVKPSETLSLTCTTSGYSFTSYYMHWIRQPPGKG |
| NO: 79 | variant VH1 | LEWIGFIDPFKVITGYNHNFRGRVTISVDRSKTQASLKLSSVTAA |
| | | DTAVYYCARRYYSDYDGYALDYWGQGTLVTVSS |
| SEQ ID | Humanization | EIQLVQSGAEVKKPGATVKISCKTSGYSFTSYYMHWVQQAPGK |
| NO: 6225 | variant VH2 | GLEWMGFIDPFKVITGYNHNFRGRVTITVDRSTTTAYMELSSLR |
| | | SEDTAVYYCARRYYSDYDGYALDYWGQGTLVTVSS |
| SEQ ID | Humanization | QIQLVQSGAEVKKTGSSVKVSCKTSGYSFTSYYMHWVRQAPG |
| NO: 6227 | variant VH3 | QALEWMGFIDPFKVITGYNHNFRGRVTITVDRSMTTAYMELSS |
| | | LRSEDTAMYYCARRYYSDYDGYALDYWGQGTLVTVSS |
| SEQ ID | Humanization | QIQLVQSGAEVKKPGASVKVSCKTSGYSFTSYYMHWVRQAPG |
| NO: 6229 | variant VH4 | QGLEWMGFIDPFKVITGYNHNFRGRVTSTVDRSITTAYMELSRL |
| | | RSDDTVVYYCARRYYSDYDGYALDYWGQGTLVTVSS |
| SEQ ID | Humanization | EVVMTQSPGTLSLSPGERATLSCRSSQSLVHSDGNTYLHWYQQ |
| NO: 6231 | variant VL1 | KPGQAPRLLIYKVSNRFSGIPDRFSGSGSGTDFTLTISRLEPEDFA |
| | | VYFCSQSTHVPPYTFGGGTKVEIK |
| SEQ ID | Humanization | EVVMTQSPATLSLSPGERATLSCRSSQSLVHSDGNTYLHWYQQ |
| NO: 6233 | variant VL2 | KPGQAPRLLIYKVSNRFSGIPARFSGSGSGTDFTLTISSLEPEDFA |
| | | VYFCSQSTHVPPYTFGGGTKVEIK |
| SEQ ID | Humanization | EVVMTQSPATLSVSPGERATLSCRSSQSLVHSDGNTYLHWYQQ |
| NO: 6235 | variant VL3 | KPGQAPRLLIYKVSNRFSGIPARFSGSGSGTEFTLTISSLQSEDFA |
| | | VYFCSQSTHVPPYTFGGGTKVEIK |
| SEQ ID | Humanization | VVWMTQSPSLLSASTGDRVTISCRSSQSLVHSDGNTYLHWYQQ |
| NO: 6237 | variant VL4 | KPGKAPELLIYKVSNRFSGVPSRFSGSGSGTDFTLTISCLQSEDF |
| | | ATYFCSQSTHVPPYTFGGGTKVEIK |

TABLE 21
Exemplary CDRs of anti-CD34 antibodies

| SEQ ID NO | Description | Sequence |
|---|---|---|
| SEQ ID NO: 6239 | VH CDR1 | FTSYYMH |
| SEQ ID NO: 6241 | VH CDR2 | FIDPFKVITGYNHNFRG |
| SEQ ID NO: 6243 | VH CDR3 | RYYSDYDGYALDY |
| SEQ ID NO: 6245 | VL CDR1 | RSSQSLVHSDGNTYLH |
| SEQ ID NO: 1236 | VL CDR2 | KVSNRFS |
| SEQ ID NO: 6246 | VL CDR3 | SQSTHVPPYT |

### Linkers

The multispecific or multifunctional molecule disclosed herein can further include a linker, e.g., a linker between one or more of: the antigen binding domain and the cytokine molecule, the antigen binding domain and the immune cell engager, the antigen binding domain and the stromal modifying moiety, the cytokine molecule and the immune cell engager, the cytokine molecule and the stromal modifying moiety, the immune cell engager and the stromal modifying moiety, the antigen binding domain and the immunoglobulin chain constant region, the cytokine molecule and the immunoglobulin chain constant region, the immune cell engager and the immunoglobulin chain constant region, or the stromal modifying moiety and the immunoglobulin chain constant region. In some embodiments, the linker is chosen from: a cleavable linker, a non-cleavable linker, a peptide linker, a flexible linker, a rigid linker, a helical linker, or a non-helical linker, or a combination thereof.

In one embodiment, the multispecific molecule can include one, two, three or four linkers, e.g., a peptide linker. In one embodiment, the peptide linker includes Gly and Ser. In some embodiments, the peptide linker is selected from GGGGS (SEQ ID NO: 6214); GGGGSGGGGS (SEQ ID NO: 6215); GGGGSGGGGSGGGGS (SEQ ID NO: 6216); and DVPSGPGGGGGSGGGGS (SEQ ID NO: 6217). In some embodiments, the peptide linker is a A(EAAAK)nA (SEQ ID NO: 6413) family of linkers (e.g., as described in Protein Eng. (2001) 14 (8): 529-532). These are stiff helical linkers with n ranging from 2-5. In some embodiments, the peptide linker is selected from AEAAAKEAAAKAAA (SEQ ID NO: 6220); AEAAAKEAAAKEAAAKAAA (SEQ ID NO: 6221); AEAAAKEAAAKEAAAKEAAAKAAA (SEQ ID NO: 77); and AEAAAKEAAAKEAAAKEAAAKEAAAKAAA(SEQ ID NO: 78).

### Nucleic Acids

Nucleic acids encoding the aforementioned multispecific or multifunctional molecules are also disclosed.

In certain embodiments, the invention features nucleic acids comprising nucleotide sequences that encode heavy and light chain variable regions and CDRs or hypervariable loops of the antibody molecules, as described herein. For example, the invention features a first and second nucleic acid encoding heavy and light chain variable regions, respectively, of an antibody molecule chosen from one or more of the antibody molecules disclosed herein. The nucleic acid can comprise a nucleotide sequence as set forth in the tables herein, or a sequence substantially identical thereto (e.g., a sequence at least about 85%, 90%, 95%, 99% or more identical thereto, or which differs by no more than 3, 6, 15, 30, or 45 nucleotides from the sequences shown in the tables herein.

In certain embodiments, the nucleic acid can comprise a nucleotide sequence encoding at least one, two, or three CDRs or hypervariable loops from a heavy chain variable region having an amino acid sequence as set forth in the tables herein, or a sequence substantially homologous thereto (e.g., a sequence at least about 85%, 90%, 95%, 99% or more identical thereto, and/or having one or more substitutions, e.g., conserved substitutions). In other embodiments, the nucleic acid can comprise a nucleotide sequence encoding at least one, two, or three CDRs or hypervariable loops from a light chain variable region having an amino acid sequence as set forth in the tables herein, or a sequence substantially homologous thereto (e.g., a sequence at least about 85%, 90%, 95%, 99% or more identical thereto, and/or having one or more substitutions, e.g., conserved substitutions). In yet another embodiment, the nucleic acid can comprise a nucleotide sequence encoding at least one, two, three, four, five, or six CDRs or hypervariable loops from heavy and light chain variable regions having an amino acid sequence as set forth in the tables herein, or a sequence substantially homologous thereto (e.g., a sequence at least about 85%, 90%, 95%, 99% or more identical thereto, and/or having one or more substitutions, e.g., conserved substitutions).

In certain embodiments, the nucleic acid can comprise a nucleotide sequence encoding at least one, two, or three CDRs or hypervariable loops from a heavy chain variable region having the nucleotide sequence as set forth in the tables herein, a sequence substantially homologous thereto (e.g., a sequence at least about 85%, 90%, 95%, 99% or more identical thereto, and/or capable of hybridizing under the stringency conditions described herein). In another embodiment, the nucleic acid can comprise a nucleotide sequence encoding at least one, two, or three CDRs or hypervariable loops from a light chain variable region having the nucleotide sequence as set forth in the tables herein, or a sequence substantially homologous thereto (e.g., a sequence at least about 85%, 90%, 95%, 99% or more identical thereto, and/or capable of hybridizing under the stringency conditions described herein). In yet another embodiment, the nucleic acid can comprise a nucleotide sequence encoding at least one, two, three, four, five, or six CDRs or hypervariable loops from heavy and light chain variable regions having the nucleotide sequence as set forth in the tables herein, or a sequence substantially homologous thereto (e.g., a sequence at least about 85%, 90%, 95%, 99% or more identical thereto, and/or capable of hybridizing under the stringency conditions described herein).

In certain embodiments, the nucleic acid can comprise a nucleotide sequence encoding a cytokine molecule, an immune cell engager, or a stromal modifying moiety disclosed herein.

In another aspect, the application features host cells and vectors containing the nucleic acids described herein. The nucleic acids may be present in a single vector or separate vectors present in the same host cell or separate host cell, as described in more detail hereinbelow.

### Vectors

Further provided herein are vectors comprising the nucleotide sequences encoding a multispecific or multifunctional molecule described herein. In one embodiment, the vectors comprise nucleotides encoding a multispecific or multifunctional molecule described herein. In one embodiment, the vectors comprise the nucleotide sequences described herein. The vectors include, but are not limited to, a virus, plasmid, cosmid, lambda phage or a yeast artificial chromosome (YAC).

Numerous vector systems can be employed. For example, one class of vectors utilizes DNA elements which are derived from animal viruses such as, for example, bovine papilloma virus, polyoma virus, adenovirus, vaccinia virus, baculovirus, retroviruses (Rous Sarcoma Virus, MMTV or MOMLV) or SV40 virus. Another class of vectors utilizes RNA elements derived from RNA viruses such as Semliki Forest virus, Eastern Equine Encephalitis virus and Flaviviruses.

Additionally, cells which have stably integrated the DNA into their chromosomes may be selected by introducing one or more markers which allow for the selection of transfected host cells. The marker may provide, for example, prototropy to an auxotrophic host, biocide resistance (e.g., antibiotics), or resistance to heavy metals such as copper, or the like. The selectable marker gene can be either directly linked to the DNA sequences to be expressed, or introduced into the same cell by cotransformation. Additional elements may also be needed for optimal synthesis of mRNA. These elements may include splice signals, as well as transcriptional promoters, enhancers, and termination signals.

Once the expression vector or DNA sequence containing the constructs has been prepared for expression, the expression vectors may be transfected or introduced into an appropriate host cell. Various techniques may be employed to achieve this, such as, for example, protoplast fusion, calcium phosphate precipitation, electroporation, retroviral transduction, viral transfection, gene gun, lipid based transfection or other conventional techniques. In the case of protoplast fusion, the cells are grown in media and screened for the appropriate activity. Methods and conditions for culturing the resulting transfected cells and for recovering the antibody molecule produced are known to those skilled in the art, and may be varied or optimized depending upon the specific expression vector and mammalian host cell employed, based upon the present description.

### Cells

In another aspect, the application features host cells and vectors containing the nucleic acids described herein. The nucleic acids may be present in a single vector or separate vectors present in the same host cell or separate host cell. The host cell can be a eukaryotic cell, e.g., a mammalian cell, an insect cell, a yeast cell, or a prokaryotic cell, e.g., E. coli. For example, the mammalian cell can be a cultured cell or a cell line. Exemplary mammalian cells include lymphocytic cell lines (e.g., NSO), Chinese hamster ovary cells (CHO), COS cells, oocyte cells, and cells from a transgenic animal, e.g., mammary epithelial cell.

The invention also provides host cells comprising a nucleic acid encoding an antibody molecule as described herein.

In one embodiment, the host cells are genetically engineered to comprise nucleic acids encoding the antibody molecule.

In one embodiment, the host cells are genetically engineered by using an expression cassette. The phrase “expression cassette,” refers to nucleotide sequences, which are capable of affecting expression of a gene in hosts compatible with such sequences. Such cassettes may include a promoter, an open reading frame with or without introns, and a termination signal. Additional factors necessary or helpful in effecting expression may also be used, such as, for example, an inducible promoter.

The invention also provides host cells comprising the vectors described herein.

The cell can be, but is not limited to, a eukaryotic cell, a bacterial cell, an insect cell, or a human cell. Suitable eukaryotic cells include, but are not limited to, Vero cells, HeLa cells, COS cells, CHO cells, HEK293 cells, BHK cells and MDCKII cells. Suitable insect cells include, but are not limited to, Sf9 cells.

### Uses and Combination Therapies

Methods described herein include treating a cancer in a subject by using a multispecific or multifunctional molecule described herein, e.g., using a pharmaceutical composition described herein. Also provided are methods for reducing or ameliorating a symptom of a cancer in a subject, as well as methods for inhibiting the growth of a cancer and/or killing one or more cancer cells. In some embodiments, the methods described herein decrease the size of a tumor and/or decrease the number of cancer cells in a subject administered with a described herein or a pharmaceutical composition described herein.

In some embodiments, the cancer is a hematological cancer. In some embodiments, the hematological cancer is a leukemia or a lymphoma. As used herein, a “hematologic cancer” refers to a tumor of the hematopoietic or lymphoid tissues, e.g., a tumor that affects blood, bone marrow, or lymph nodes. Exemplary hematologic malignancies include, but are not limited to, leukemia (e.g., acute lymphoblastic leukemia (ALL), acute myeloid leukemia (AML), chronic lymphocytic leukemia (CLL), chronic myelogenous leukemia (CML), hairy cell leukemia, acute monocytic leukemia (AMoL), chronic myelomonocytic leukemia (CMML), juvenile myelomonocytic leukemia (JMML), or large granular lymphocytic leukemia), lymphoma (e.g., AIDS-related lymphoma, cutaneous T-cell lymphoma, Hodgkin lymphoma (e.g., classical Hodgkin lymphoma or nodular lymphocyte-predominant Hodgkin lymphoma), mycosis fungoides, non-Hodgkin lymphoma (e.g., B-cell non-Hodgkin lymphoma (e.g., Burkitt lymphoma, small lymphocytic lymphoma (CLL/SLL), diffuse large B-cell lymphoma, follicular lymphoma, immunoblastic large cell lymphoma, precursor B-lymphoblastic lymphoma, or mantle cell lymphoma) or T-cell non-Hodgkin lymphoma (mycosis fungoides, anaplastic large cell lymphoma, or precursor T-lymphoblastic lymphoma)), primary central nervous system lymphoma, Sézary syndrome, Waldenstrom macroglobulinemia), chronic myeloproliferative neoplasm, Langerhans cell histiocytosis, multiple myeloma/plasma cell neoplasm, myelodysplastic syndrome, or myelodysplastic/myeloproliferative neoplasm.

In some embodiments, the cancer is a myeloproliferative neoplasm, e.g., primary or idiopathic myelofibrosis (MF), essential thrombocytosis (ET), polycythemia vera (PV), or chronic myelogenous leukemia (CML). In some embodiments, the cancer is myelofibrosis. In some embodiments, the subject has myelofibrosis. In some embodiments, the subject has a calreticulin mutation, e.g., a calreticulin mutation disclosed herein. In some embodiments, the subject does not have the JAK2-V617F mutation. In some embodiments, the subject has the JAK2-V617F mutation. In some embodiments, the subject has a MPL mutation. In some embodiments, the subject does not have a MPL mutation.

In some embodiments, the cancer is a solid cancer. Exemplary solid cancers include, but are not limited to, ovarian cancer, rectal cancer, stomach cancer, testicular cancer, cancer of the anal region, uterine cancer, colon cancer, rectal cancer, renal-cell carcinoma, liver cancer, non-small cell carcinoma of the lung, cancer of the small intestine, cancer of the esophagus, melanoma, Kaposi's sarcoma, cancer of the endocrine system, cancer of the thyroid gland, cancer of the parathyroid gland, cancer of the adrenal gland, bone cancer, pancreatic cancer, skin cancer, cancer of the head or neck, cutaneous or intraocular malignant melanoma, uterine cancer, brain stem glioma, pituitary adenoma, epidermoid cancer, carcinoma of the cervix squamous cell cancer, carcinoma of the fallopian tubes, carcinoma of the endometrium, carcinoma of the vagina, sarcoma of soft tissue, cancer of the urethra, carcinoma of the vulva, cancer of the penis, cancer of the bladder, cancer of the kidney or ureter, carcinoma of the renal pelvis, spinal axis tumor, neoplasm of the central nervous system (CNS), primary CNS lymphoma, tumor angiogenesis, metastatic lesions of said cancers, or combinations thereof.

In some embodiments, the multispecific or multifunctional molecules (or pharmaceutical composition) are administered in a manner appropriate to the disease to be treated or prevented. The quantity and frequency of administration will be determined by such factors as the condition of the patient, and the type and severity of the patient's disease. Appropriate dosages may be determined by clinical trials. For example, when “an effective amount” or “a therapeutic amount” is indicated, the precise amount of the pharmaceutical composition (or multispecific or multifunctional molecules) to be administered can be determined by a physician with consideration of individual differences in tumor size, extent of infection or metastasis, age, weight, and condition of the subject. In some embodiments, the pharmaceutical composition described herein can be administered at a dosage of 10⁴ to 10⁹ cells/kg body weight, e.g., 10⁵ to 10⁶ cells/kg body weight, including all integer values within those ranges. In some embodiments, the pharmaceutical composition described herein can be administered multiple times at these dosages. In some embodiments, the pharmaceutical composition described herein can be administered using infusion techniques described in immunotherapy (see, e.g., Rosenberg et al., New Eng. J. of Med. 319:1676, 1988).

In some embodiments, the multispecific or multifunctional molecules or pharmaceutical composition is administered to the subject parenterally. In some embodiments, the cells are administered to the subject intravenously, subcutaneously, intratumorally, intranodally, intramuscularly, intradermally, or intraperitoneally. In some embodiments, the cells are administered, e.g., injected, directly into a tumor or lymph node. In some embodiments, the cells are administered as an infusion (e.g., as described in Rosenberg et al., New Eng. J. of Med. 319:1676, 1988) or an intravenous push. In some embodiments, the cells are administered as an injectable depot formulation.

In some embodiments, the subject is a mammal. In some embodiments, the subject is a human, monkey, pig, dog, cat, cow, sheep, goat, rabbit, rat, or mouse. In embodiments, the subject is a human. In some embodiments, the subject is a pediatric subject, e.g., less than 18 years of age, e.g., less than 17, 16, 15, 14, 13, 12, 11, 10, 9, 8, 7, 6, 5, 4, 3, 2, 1 or less years of age. In some embodiments, the subject is an adult, e.g., at least 18 years of age, e.g., at least 19, 20, 21, 22, 23, 24, 25, 25-30, 30-35, 35-40, 40-50, 50-60, 60-70, 70-80, or 80-90 years of age.

### Combination Therapies

The multispecific or multifunctional molecules disclosed herein can be used in combination with a second therapeutic agent or procedure.

In some embodiments, the multispecific or multifunctional molecule and the second therapeutic agent or procedure are administered/performed after a subject has been diagnosed with a cancer, e.g., before the cancer has been eliminated from the subject. In some embodiments, the multispecific or multifunctional molecule and the second therapeutic agent or procedure are administered/performed simultaneously or concurrently. For example, the delivery of one treatment is still occurring when the delivery of the second commences, e.g., there is an overlap in administration of the treatments. In other embodiments, the multispecific or multifunctional molecule and the second therapeutic agent or procedure are administered/performed sequentially. For example, the delivery of one treatment ceases before the delivery of the other treatment begins.

In some embodiments, combination therapy can lead to more effective treatment than monotherapy with either agent alone. In some embodiments, the combination of the first and second treatment is more effective (e.g., leads to a greater reduction in symptoms and/or cancer cells) than the first or second treatment alone. In some embodiments, the combination therapy permits use of a lower dose of the first or the second treatment compared to the dose of the first or second treatment normally required to achieve similar effects when administered as a monotherapy. In some embodiments, the combination therapy has a partially additive effect, wholly additive effect, or greater than additive effect.

In one embodiment, the multispecific or multifunctional molecule is administered in combination with a therapy, e.g., a cancer therapy (e.g., one or more of anti-cancer agents, immunotherapy, photodynamic therapy (PDT), surgery and/or radiation). The terms “chemotherapeutic,” “chemotherapeutic agent,” and “anti-cancer agent” are used interchangeably herein. The administration of the multispecific or multifunctional molecule and the therapy, e.g., the cancer therapy, can be sequential (with or without overlap) or simultaneous. Administration of the multispecific or multifunctional molecule can be continuous or intermittent during the course of therapy (e.g., cancer therapy). Certain therapies described herein can be used to treat cancers and non-cancerous diseases. For example, PDT efficacy can be enhanced in cancerous and non-cancerous conditions (e.g., tuberculosis) using the methods and compositions described herein (reviewed in, e.g., Agostinis, P. et al. (2011) CA Cancer J. Clin. 61:250-281).

### Anti-Cancer Therapies

In other embodiments, the multispecific or multifunctional molecule is administered in combination with a low or small molecular weight chemotherapeutic agent. Exemplary low or small molecular weight chemotherapeutic agents include, but not limited to, 13-cis-retinoic acid (isotretinoin, ACCUTANE®), 2-CdA (2-chlorodeoxyadenosine, cladribine, LEUSTATIN™), 5-azacitidine (azacitidine, VIDAZA®), 5-fluorouracil (5-FU, fluorouracil, ADRUCIL®), 6-mercaptopurine (6-MP, mercaptopurine, PURINETHOL®), 6-TG (6-thioguanine, thioguanine, THIOGUANINE TABLOID®), abraxane (paclitaxel protein-bound), actinomycin-D (dactinomycin, COSMEGEN®), alitretinoin (PANRETIN®), all-transretinoic acid (ATRA, tretinoin, VESANOID®), altretamine (hexamethylmelamine, HMM, HEXALEN®), amethopterin (methotrexate, methotrexate sodium, MTX, TREXALL™ RHEUMATREX®), amifostine (ETHYOL®), arabinosylcytosine (Ara-C, cytarabine, CYTOSAR-U®), arsenic trioxide (TRISENOX®), asparaginase (Erwinia L-asparaginase, L-asparaginase, ELSPAR®, KIDROLASE®), BCNU (carmustine, BiCNUo), bendamustine (TREANDA®), bexarotene (TARGRETIN®), bleomycin (BLENOXANE®), busulfan (BUSULFEX®, MYLERAN®), calcium leucovorin (Citrovorum Factor, folinic acid, leucovorin), camptothecin-11 (CPT-11, irinotecan, CAMPTOSAR®), capecitabine (XELODA®), carboplatin (PARAPLATIN®), carmustine wafer (prolifeprospan 20 with carmustine implant, GLIADEL® wafer), CCI-779 (temsirolimus, TORISEL®), CCNU (lomustine, CeeNU), CDDP (cisplatin, PLATINOL®, PLATINOL-AQ®), chlorambucil (leukeran), cyclophosphamide (CYTOXAN®, NEOSAR®), dacarbazine (DIC, DTIC, imidazole carboxamide, DTIC-DOME®), daunomycin (daunorubicin, daunorubicin hydrochloride, rubidomycin hydrochloride, CERUBIDINE®), decitabine (DACOGEN®), dexrazoxane (ZINECARD®), DHAD (mitoxantrone, NOVANTRONE®), docetaxel (TAXOTERE®), doxorubicin (ADRIAMYCIN®, RUBEX®), epirubicin (ELLENCE™), estramustine (EMCYT®), etoposide (VP-16, etoposide phosphate, TOPOSAR®, VEPESID®, ETOPOPHOS®), floxuridine (FUDR®), fludarabine (FLUDARA®), fluorouracil (cream) (CARAC™, EFUDEX®, FLUOROPLEX®), gemcitabine (GEMZAR®), hydroxyurea (HYDREA®, DROXIA™, MYLOCEL™), idarubicin (IDAMYCIN®), ifosfamide (IFEX®), ixabepilone (IXEMPRA™), LCR (leurocristine, vincristine, VCR, ONCOVIN®, VINCASAR PFS®), L-PAM (L-sarcolysin, melphalan, phenylalanine mustard, ALKERAN®), mechlorethamine (mechlorethamine hydrochloride, mustine, nitrogen mustard, MUSTARGEN®), mesna (MESNEX™) mitomycin (mitomycin-C, MTC, MUTAMYCIN®), nelarabine (ARRANON®), oxaliplatin (ELOXATIN™), paclitaxel (TAXOL®, ONXAL™), pegaspargase (PEG-L-asparaginase, ONCOSPAR®), PEMETREXED (ALIMTA®), pentostatin (NIPENT®), procarbazine (MATULANE®), streptozocin (ZANOSAR®), temozolomide (TEMODAR®), teniposide (VM-26, VUMON®), TESPA (thiophosphoamide, thiotepa, TSPA, THIOPLEX®), topotecan (HYCAMTIN®), vinblastine (vinblastine sulfate, vincaleukoblastine, VLB, ALKABAN-AQ®, VELBAN®), vinorelbine (vinorelbine tartrate, NAVELBINE®), and vorinostat (ZOLINZA®).

In another embodiment, the multispecific or multifunctional molecule is administered in conjunction with a biologic. Biologics useful in the treatment of cancers are known in the art and a binding molecule of the invention may be administered, for example, in conjunction with such known biologics. For example, the FDA has approved the following biologics for the treatment of breast cancer: HERCEPTIN® (trastuzumab, Genentech Inc., South San Francisco, Calif.; a humanized monoclonal antibody that has anti-tumor activity in HER2-positive breast cancer); FASLODEX® (fulvestrant, AstraZeneca Pharmaceuticals, LP, Wilmington, Del.; an estrogen-receptor antagonist used to treat breast cancer); ARIMIDEX® (anastrozole, AstraZeneca Pharmaceuticals, LP; a nonsteroidal aromatase inhibitor which blocks aromatase, an enzyme needed to make estrogen); Aromasin® (exemestane, Pfizer Inc., New York, N.Y.; an irreversible, steroidal aromatase inactivator used in the treatment of breast cancer); FEMARA® (letrozole, Novartis Pharmaceuticals, East Hanover, N.J.; a nonsteroidal aromatase inhibitor approved by the FDA to treat breast cancer); and NOLVADEX® (tamoxifen, AstraZeneca Pharmaceuticals, LP; a nonsteroidal antiestrogen approved by the FDA to treat breast cancer). Other biologics with which the binding molecules of the invention may be combined include: AVASTIN® (bevacizumab, Genentech Inc.; the first FDA-approved therapy designed to inhibit angiogenesis); and ZEVALIN® (ibritumomab tiuxetan, Biogen Idec, Cambridge, Mass.; a radiolabeled monoclonal antibody currently approved for the treatment of B-cell lymphomas).

In addition, the FDA has approved the following biologics for the treatment of colorectal cancer: AVASTIN®; ERBITUX® (cetuximab, ImClone Systems Inc., New York, N.Y., and Bristol-Myers Squibb, New York, N.Y.; is a monoclonal antibody directed against the epidermal growth factor receptor (EGFR)); GLEEVEC® (imatinib mesylate; a protein kinase inhibitor); and ERGAMISOL® (levamisole hydrochloride, Janssen Pharmaceutica Products, LP, Titusville, N.J.; an immunomodulator approved by the FDA in 1990 as an adjuvant treatment in combination with 5-fluorouracil after surgical resection in patients with Dukes' Stage C colon cancer).

For the treatment of lung cancer, exemplary biologics include TARCEVA® (erlotinib HCL, OSI Pharmaceuticals Inc., Melville, N.Y.; a small molecule designed to target the human epidermal growth factor receptor 1 (HER1) pathway).

For the treatment of multiple myeloma, exemplary biologics include VELCADE® Velcade (bortezomib, Millennium Pharmaceuticals, Cambridge Mass.; a proteasome inhibitor). Additional biologics include THALIDOMID® (thalidomide, Clegene Corporation, Warren, N.J.; an immunomodulatory agent and appears to have multiple actions, including the ability to inhibit the growth and survival of myeloma cells and anti-angiogenesis).

Additional exemplary cancer therapeutic antibodies include, but are not limited to, 3F8, abagovomab, adecatumumab, afutuzumab, alacizumab pegol, alemtuzumab (CAMPATH®, MABCAMPATH®), altumomab pentetate (HYBRI-CEAKER®), anatumomab mafenatox, anrukinzumab (IMA-638), apolizumab, arcitumomab (CEA-SCAN®), bavituximab, bectumomab (LYMPHOSCAN®), belimumab (BENLYSTA®, LYMPHOSTAT-B®), besilesomab (SCINTIMUN®), bevacizumab (AVASTIN®), bivatuzumab mertansine, blinatumomab, brentuximab vedotin, cantuzumab mertansine, capromab pendetide (PROSTASCINT®), catumaxomab (REMOVAB®), CC49, cetuximab (C225, ERBITUX®), citatuzumab bogatox, cixutumumab, clivatuzumab tetraxetan, conatumumab, dacetuzumab, denosumab (PROLIA®), detumomab, ecromeximab, edrecolomab (PANOREX®), elotuzumab, epitumomab cituxetan, epratuzumab, ertumaxomab (REXOMUN®), etaracizumab, farletuzumab, figitumumab, fresolimumab, galiximab, gemtuzumab ozogamicin (MYLOTARG®), girentuximab, glembatumumab vedotin, ibritumomab (ibritumomab tiuxetan, ZEVALIN®), igovomab (INDIMACIS-125®), intetumumab, inotuzumab ozogamicin, ipilimumab, iratumumab, labetuzumab (CEA-CIDE®), lexatumumab, lintuzumab, lucatumumab, lumiliximab, mapatumumab, matuzumab, milatuzumab, minretumomab, mitumomab, nacolomab tafenatox, naptumomab estafenatox, necitumumab, nimotuzumab (THERACIM®, THERALOC®), nofetumomab merpentan (VERLUMA®), ofatumumab (ARZERRA®), olaratumab, oportuzumab monatox, oregovomab (OVAREX®), panitumumab (VECTIBIX®), pemtumomab (THERAGYN®), pertuzumab (OMNITARG®), pintumomab, pritumumab, ramucirumab, ranibizumab (LUCENTIS®), rilotumumab, rituximab (MABTHERA®, RITUXAN®), robatumumab, satumomab pendetide, sibrotuzumab, siltuximab, sontuzumab, tacatuzumab tetraxetan (AFP-CIDE®), taplitumomab paptox, tenatumomab, TGN1412, ticilimumab (tremelimumab), tigatuzumab, TNX-650, tositumomab (BEXXAR®), trastuzumab (HERCEPTIN®), tremelimumab, tucotuzumab celmoleukin, veltuzumab, volociximab, votumumab (HUMASPECT®), zalutumumab (HUMAX-EGFR®), and zanolimumab (HUMAX-CD4®).

In other embodiments, the multispecific or multifunctional molecule is administered in combination with a viral cancer therapeutic agent. Exemplary viral cancer therapeutic agents include, but not limited to, vaccinia virus (vvDD-CDSR), carcinoembryonic antigen-expressing measles virus, recombinant vaccinia virus (TK-deletion plus GM-CSF), Seneca Valley virus-001, Newcastle virus, coxsackie virus A21, GL-ONC1, EBNA1 C-terminal/LMP2 chimeric protein-expressing recombinant modified vaccinia Ankara vaccine, carcinoembryonic antigen-expressing measles virus, G207 oncolytic virus, modified vaccinia virus Ankara vaccine expressing p53, OncoVEX GM-CSF modified herpes-simplex 1 virus, fowlpox virus vaccine vector, recombinant vaccinia prostate-specific antigen vaccine, human papillomavirus 16/18 L1 virus-like particle/AS04 vaccine, MVA-EBNA1/LMP2 Inj. vaccine, quadrivalent HPV vaccine, quadrivalent human papillomavirus (types 6, 11, 16, 18) recombinant vaccine (GARDASIL®), recombinant fowlpox-CEA(6D)/TRICOM vaccine; recombinant vaccinia-CEA(6D)-TRICOM vaccine, recombinant modified vaccinia Ankara-5T4 vaccine, recombinant fowlpox-TRICOM vaccine, oncolytic herpes virus NV1020, HPV L1 VLP vaccine V504, human papillomavirus bivalent (types 16 and 18) vaccine (CERVARIX®), herpes simplex virus HF10, Ad5CMV-p53 gene, recombinant vaccinia DF3/MUC1 vaccine, recombinant vaccinia-MUC-1 vaccine, recombinant vaccinia-TRICOM vaccine, ALVAC MART-1 vaccine, replication-defective herpes simplex virus type I (HSV-1) vector expressing human Preproenkephalin (NP2), wild-type reovirus, reovirus type 3 Dearing (REOLYSIN®), oncolytic virus HSV1716, recombinant modified vaccinia Ankara (MVA)-based vaccine encoding Epstein-Barr virus target antigens, recombinant fowlpox-prostate specific antigen vaccine, recombinant vaccinia prostate-specific antigen vaccine, recombinant vaccinia-B7.1 vaccine, rAd-p53 gene, Ad5-delta24RGD, HPV vaccine 580299, JX-594 (thymidine kinase-deleted vaccinia virus plus GM-CSF), HPV-16/18 L1/AS04, fowlpox virus vaccine vector, vaccinia-tyrosinase vaccine, MEDI-517 HPV-16/18 VLP AS04 vaccine, adenoviral vector containing the thymidine kinase of herpes simplex virus TK99UN, HspE7, FP253/Fludarabine, ALVAC(2) melanoma multi-antigen therapeutic vaccine, ALVAC-hB7.1, canarypox-hIL-12 melanoma vaccine, Ad-REIC/Dkk-3, rAd-IFN SCH 721015, TIL-Ad-INFg, Ad-ISF35, and coxsackievirus A21 (CVA21, CAVATAK®).

In other embodiments, the multispecific or multifunctional molecule is administered in combination with a nanopharmaceutical. Exemplary cancer nanopharmaceuticals include, but not limited to, ABRAXANE® (paclitaxel bound albumin nanoparticles), CRLX101 (CPT conjugated to a linear cyclodextrin-based polymer), CRLX288 (conjugating docetaxel to the biodegradable polymer poly (lactic-co-glycolic acid)), cytarabine liposomal (liposomal Ara-C, DEPOCYT^{M}), daunorubicin liposomal (DAUNOXOME®), doxorubicin liposomal (DOXIL®, CAELYX®), encapsulated-daunorubicin citrate liposome (DAUNOXOME®), and PEG anti-VEGF aptamer (MACUGEN®).

In some embodiments, the multispecific or multifunctional molecule is administered in combination with paclitaxel or a paclitaxel formulation, e.g., TAXOL®, protein-bound paclitaxel (e.g., ABRAXANE®). Exemplary paclitaxel formulations include, but are not limited to, nanoparticle albumin-bound paclitaxel (ABRAXANE®, marketed by Abraxis Bioscience), docosahexaenoic acid bound-paclitaxel (DHA-paclitaxel, Taxoprexin, marketed by Protarga), polyglutamate bound-paclitaxel (PG-paclitaxel, paclitaxel poliglumex, CT-2103, XYOTAX, marketed by Cell Therapeutic), the tumor-activated prodrug (TAP), ANG105 (Angiopep-2 bound to three molecules of paclitaxel, marketed by ImmunoGen), paclitaxel-EC-1 (paclitaxel bound to the erbB2-recognizing peptide EC-1; see Li et al., Biopolymers (2007) 87:225-230), and glucose-conjugated paclitaxel (e.g., 2′-paclitaxel methyl 2-glucopyranosyl succinate, see Liu et al., Bioorganic & Medicinal Chemistry Letters (2007) 17:617-620).

Exemplary RNAi and antisense RNA agents for treating cancer include, but not limited to, CALAA-01, siG12D LODER (Local Drug EluteR), and ALN-VSP02.

Other cancer therapeutic agents include, but not limited to, cytokines (e.g., aldesleukin (IL-2, Interleukin-2, PROLEUKIN®), alpha Interferon (IFN-alpha, Interferon alfa, INTRON® A (Interferon alfa-2b), ROFERON-A® (Interferon alfa-2a)), Epoetin alfa (PROCRIT®), filgrastim (G-CSF, Granulocyte-Colony Stimulating Factor, NEUPOGEN®), GM-CSF (Granulocyte Macrophage Colony Stimulating Factor, sargramostim, LEUKINE™), IL-11(Interleukin-11, oprelvekin, NEUMEGA®), Interferon alfa-2b (PEG conjugate) (PEG interferon, PEG-INTRON™), and pegfilgrastim (NEULASTA™)), hormone therapy agents (e.g., aminoglutethimide (CYTADREN®), anastrozole (ARIMIDEX®), bicalutamide (CASODEX®), exemestane (AROMASIN®), fluoxymesterone (HALOTESTIN®), flutamide (EULEXIN®), fulvestrant (FASLODEX®), goserelin (ZOLADEX®), letrozole (FEMARA®), leuprolide (ELIGARD™, LUPRON®, LUPRON DEPOT®, VIADUR™) megestrol (megestrol acetate, MEGACE®), nilutamide (ANANDRON®, NILANDRON®), octreotide (octreotide acetate, SANDOSTATIN®, SANDOSTATIN LAR®), raloxifene (EVISTA®), romiplostim (NPLATE®), tamoxifen (NOVALDEX®), and toremifene (FARESTON®)), phospholipase A2 inhibitors (e.g., anagrelide (AGRYLIN®)), biologic response modifiers (e.g., BCG (THERACYS®, TICE®), and Darbepoetin alfa (ARANESP®)), target therapy agents (e.g., bortezomib (VELCADE®), dasatinib (SPRYCEL™), denileukin diftitox (ONTAK®), erlotinib (TARCEVA®), everolimus (AFINITOR®), gefitinib (IRESSA®), imatinib mesylate (STI-571, GLEEVEC™), lapatinib (TYKERB®), sorafenib (NEXAVAR®), and SU11248 (sunitinib, SUTENT®)), immunomodulatory and antiangiogenic agents (e.g., CC-5013 (lenalidomide, REVLIMID®), and thalidomide (THALOMID®)), glucocorticosteroids (e.g., cortisone (hydrocortisone, hydrocortisone sodium phosphate, hydrocortisone sodium succinate, ALA-CORT®, HYDROCORT ACETATE®, hydrocortone phosphate LANACORT®, SOLU-CORTEF®), decadron (dexamethasone, dexamethasone acetate, dexamethasone sodium phosphate, DEXASONE®, DIODEX®, HEXADROL®, MAXIDEX®), methylprednisolone (6-methylprednisolone, methylprednisolone acetate, methylprednisolone sodium succinate, DURALONE®, MEDRALONE®, MEDROL®, M-PREDNISOL®, SOLU-MEDROL®), prednisolone (DELTA-CORTEF®, ORAPRED®, PEDIAPRED®, PRELONE®), and prednisone (DELTASONE®, LIQUID PRED®, METICORTEN®, ORASONE®)), and bisphosphonates (e.g., pamidronate (AREDIA®), and zoledronic acid (ZOMETA®))

In some embodiments, the multispecific or multifunctional molecule is used in combination with a tyrosine kinase inhibitor (e.g., a receptor tyrosine kinase (RTK) inhibitor). Exemplary tyrosine kinase inhibitor include, but are not limited to, an epidermal growth factor (EGF) pathway inhibitor (e.g., an epidermal growth factor receptor (EGFR) inhibitor), a vascular endothelial growth factor (VEGF) pathway inhibitor (e.g., an antibody against VEGF, a VEGF trap, a vascular endothelial growth factor receptor (VEGFR) inhibitor (e.g., a VEGFR-1 inhibitor, a VEGFR-2 inhibitor, a VEGFR-3 inhibitor)), a platelet derived growth factor (PDGF) pathway inhibitor (e.g., a platelet derived growth factor receptor (PDGFR) inhibitor (e.g., a PDGFR-β inhibitor)), a RAF-1 inhibitor, a KIT inhibitor and a RET inhibitor. In some embodiments, the anti-cancer agent used in combination with the AHCM agent is selected from the group consisting of: axitinib (AG013736), bosutinib (SKI-606), cediranib (RECENTIN™ AZD2171), dasatinib (SPRYCEL®, BMS-354825), erlotinib (TARCEVA®), gefitinib (IRESSA®), imatinib (Gleevec®, CGP57148B, STI-571), lapatinib (TYKERB®, TYVERB®), lestaurtinib (CEP-701), neratinib (HKI-272), nilotinib (TASIGNA®), semaxanib (semaxinib, SU5416), sunitinib (SUTENT®, SU11248), toceranib (PALLADIA®), vandetanib (ZACTIMA®, ZD6474), vatalanib (PTK787, PTK/ZK), trastuzumab (HERCEPTIN®), bevacizumab (AVASTIN®), rituximab (RITUXAN®), cetuximab (ERBITUX®), panitumumab (VECTIBIX®), ranibizumab (Lucentis®), nilotinib (TASIGNA®), sorafenib (NEXAVAR®), alemtuzumab (CAMPATH®), gemtuzumab ozogamicin (MYLOTARG®), ENMD-2076, PCI-32765, AC220, dovitinib lactate (TKI258, CHIR-258), BIBW 2992 (TOVOK™), SGX523, PF-04217903, PF-02341066, PF-299804, BMS-777607, ABT-869, MP470, BIBF 1120 (VARGATEF®), AP24534, JNJ-26483327, MGCD265, DCC-2036, BMS-690154, CEP-11981, tivozanib (AV-951), OSI-930, MM-121, XL-184, XL-647, XL228, AEE788, AG-490, AST-6, BMS-599626, CUDC-101, PD153035, pelitinib (EKB-569), vandetanib (zactima), WZ3146, WZ4002, WZ8040, ABT-869 (linifanib), AEE788, AP24534 (ponatinib), AV-951 (tivozanib), axitinib, BAY 73-4506 (regorafenib), brivanib alaninate (BMS-582664), brivanib (BMS-540215), cediranib (AZD2171), CHIR-258 (dovitinib), CP 673451, CYC116, E7080, Ki8751, masitinib (AB1010), MGCD-265, motesanib diphosphate (AMG-706), MP-470, OSI-930, Pazopanib Hydrochloride, PD173074, Sorafenib Tosylate (Bay 43-9006), SU 5402, TSU-68 (SU6668), vatalanib, XL880 (GSK1363089, EXEL-2880). Selected tyrosine kinase inhibitors are chosen from sunitinib, erlotinib, gefitinib, or sorafenib. In one embodiment, the tyrosine kinase inhibitor is sunitinib.

In one embodiment, the multispecific or multifunctional molecule is administered in combination with one of more of: an anti-angiogenic agent, or a vascular targeting agent or a vascular disrupting agent. Exemplary anti-angiogenic agents include, but are not limited to, VEGF inhibitors (e.g., anti-VEGF antibodies (e.g., bevacizumab); VEGF receptor inhibitors (e.g., itraconazole); inhibitors of cell proliferatin and/or migration of endothelial cells (e.g., carboxyamidotriazole, TNP-470); inhibitors of angiogenesis stimulators (e.g., suramin), among others. A vascular-targeting agent (VTA) or vascular disrupting agent (VDA) is designed to damage the vasculature (blood vessels) of cancer tumors causing central necrosis (reviewed in, e.g., Thorpe, P. E. (2004) Clin. Cancer Res. Vol. 10:415-427). VTAs can be small-molecule. Exemplary small-molecule VTAs include, but are not limited to, microtubule destabilizing drugs (e.g., combretastatin A-4 disodium phosphate (CA4P), ZD6126, AVE8062, Oxi 4503); and vadimezan (ASA404).

### Immune Checkpoint Inhibitors

In other embodiments, methods described herein comprise use of an immune checkpoint inhibitor in combination with the multispecific or multifunctional molecule. The methods can be used in a therapeutic protocol in vivo.

In some embodiments, an immune checkpoint inhibitor inhibits a checkpoint molecule. Exemplary checkpoint molecules include but are not limited to CTLA4, PD1, PD-L1, PD-L2, TIM3, LAG3, CD160, 2B4, CD80, CD86, B7-H3 (CD276), B7-H4 (VTCN1), HVEM (TNFRSF14 or CD270), BTLA, KIR, MHC class I, MHC class II, GAL9, VISTA, BTLA, TIGIT, LAIRI, and A2aR. See, e.g., Pardoll. Nat. Rev. Cancer 12.4(2012):252-64, incorporated herein by reference.

In some embodiments, the immune checkpoint inhibitor is a PD-1 inhibitor, e.g., an anti-PD-1 antibody such as Nivolumab, Pembrolizumab or Pidilizumab. Nivolumab (also called MDX-1106, MDX-1106-04, ONO-4538, or BMS-936558) is a fully human IgG4 monoclonal antibody that specifically inhibits PD1. See, e.g., U.S. Pat. No. 8,008,449 and WO2006/121168. Pembrolizumab (also called Lambrolizumab, MK-3475, MK03475, SCH-900475 or KEYTRUDA®; Merck) is a humanized IgG4 monoclonal antibody that binds to PD-1. See, e.g., Hamid, O. et al. (2013) New England Journal of Medicine 369 (2): 134-44, U.S. Pat. No. 8,354,509 and WO2009/114335. Pidilizumab (also called CT-011 or Cure Tech) is a humanized IgGik monoclonal antibody that binds to PDi. See, e.g., WO2009/101611. In one embodiment, the inhibitor of PD-1 is an antibody molecule having a sequence substantially identical or similar thereto, e.g., a sequence at least 85%, 90%, 95% identical or higher to the sequence of Nivolumab, Pembrolizumab or Pidilizumab. Additional anti-PD1 antibodies, e.g., AMP 514 (Amplimmune), are described, e.g., in U.S. Pat. No. 8,609,089, US 2010028330, and/or US 20120114649.

In some embodiments, the PD-1 inhibitor is an immunoadhesin, e.g., an immunoadhesin comprising an extracellular/PD-1 binding portion of a PD-1 ligand (e.g., PD-L1 or PD-L2) that is fused to a constant region (e.g., an Fc region of an immunoglobulin). In some embodiments, the PD-1 inhibitor is AMP-224 (B7-DCIg, e.g., described in WO2011/066342 and WO2010/027827), a PD-L2 Fc fusion soluble receptor that blocks the interaction between B7-H1 and PD-1.

In some embodiments, the immune checkpoint inhibitor is a PD-L1 inhibitor, e.g., an antibody molecule. In some embodiments, the PD-L1 inhibitor is YW243.55.S70, MPDL3280A, MEDI-4736, MSB-0010718C, or MDX-1105. In some embodiments, the anti-PD-L1 antibody is MSB0010718C (also called A09-246-2; Merck Serono), which is a monoclonal antibody that binds to PD-L1. Exemplary humanized anti-PD-L1 antibodies are described, e.g., in WO2013/079174. In one embodiment, the PD-L1 inhibitor is an anti-PD-L1 antibody, e.g., YW243.55.S70. The YW243.55.S70 antibody is described, e.g., in WO 2010/077634. In one embodiment, the PD-L1 inhibitor is MDX-1105 (also called BMS-936559), which is described, e.g., in WO2007/005874. In one embodiment, the PD-L1 inhibitor is MDPL3280A (Genentech/Roche), which is a human Fc-optimized IgG1 monoclonal antibody against PD-L1. See, e.g., U.S. Pat. No. 7,943,743 and U.S Publication No.: 20120039906. In one embodiment, the inhibitor of PD-L1 is an antibody molecule having a sequence substantially identical or similar thereto, e.g., a sequence at least 85%, 90%, 95% identical or higher to the sequence of YW243.55.S70, MPDL3280A, MEDI-4736, MSB-0010718C, or MDX-1105.

In some embodiments, the immune checkpoint inhibitor is a PD-L2 inhibitor, e.g., AMP-224 (which is a PD-L2 Fc fusion soluble receptor that blocks the interaction between PD1 and B7-H1. See, e.g., WO2010/027827 and WO2011/066342.

In one embodiment, the immune checkpoint inhibitor is a LAG-3 inhibitor, e.g., an anti LAG-3 antibody molecule. In some embodiments, the anti-LAG-3 antibody is BMS-986016 (also called BMS986016; Bristol-Myers Squibb). BMS-986016 and other humanized anti-LAG-3 antibodies are described, e.g., in US 2011/0150892, WO2010/019570, and WO2014/008218.

In some embodiments, the immune checkpoint inhibitor is a TIM-3 inhibitor, e.g., anti-TIM3 antibody molecule, e.g., described in U.S. Pat. No. 8,552,156, WO 2011/155607, EP 2581113 and U.S Publication No.: 2014/044728.

In some embodiments, the immune checkpoint inhibitor is a CTLA-4 inhibitor, e.g., anti-CTLA-4 antibody molecule. Exemplary anti-CTLA4 antibodies include Tremelimumab (IgG2 monoclonal antibody from Pfizer, formerly known as ticilimumab, CP-675,206); and Ipilimumab (also called MDX-010, CAS No. 477202-00-9). Other exemplary anti-CTLA-4 antibodies are described, e.g., in U.S. Pat. No. 5,811,097.

### CRS Grading

In some embodiments, the compositions described herein may induce lower levels of cytokine release syndrome (CRS) and/or may have a lower chance of causing (e.g., may not cause) CRS compared to other compositions. In some embodiments, CRS can be graded in severity from 1-5 as follows. Grades 1-3 are less than severe CRS. Grades 4-5 are severe CRS. For Grade 1 CRS, only symptomatic treatment is needed (e.g., nausea, fever, fatigue, myalgias, malaise, headache) and symptoms are not life threatening. For Grade 2 CRS, the symptoms require moderate intervention and generally respond to moderate intervention. Subjects having Grade 2 CRS develop hypotension that is responsive to either fluids or one low-dose vasopressor; or they develop grade 2 organ toxicity or mild respiratory symptoms that are responsive to low flow oxygen (<40% oxygen). In Grade 3 CRS subjects, hypotension generally cannot be reversed by fluid therapy or one low-dose vasopressor. These subjects generally require more than low flow oxygen and have grade 3 organ toxicity (e.g., renal or cardiac dysfunction or coagulopathy) and/or grade 4 transaminitis. Grade 3 CRS subjects require more aggressive intervention, e.g., oxygen of 40% or higher, high dose vasopressor(s), and/or multiple vasopressors. Grade 4 CRS subjects suffer from immediately life-threatening symptoms, including grade 4 organ toxicity or a need for mechanical ventilation. Grade 4 CRS subjects generally do not have transaminitis. In Grade 5 CRS subjects, the toxicity causes death. Sets of criteria for grading CRS are provided herein as Table 39, Table 40, and Table 41. Unless otherwise specified, CRS as used herein refers to CRS according to the criteria of Table 40.

In some embodiments, CRS is graded according to Table 39:

TABLE 39
CRS grading

| | |
|---|---|
| Gr1 | Supportive care only |
| Gr2 | IV therapies +/− hospitalization. |
| Gr3 | Hypotension requiring IV fluids or low-dose vasoactives or |
| | hypoxemia requiring oxygen, CPAP, or BIPAP. |
| Gr4 | Hypotension requiring high-dose vasoactives or hypoxemia |
| | requiring mechanical ventilation. |
| Gr 5 | Death |

TABLE 40
CTCAE v 4.0 CRS grading scale

| CRS | |
|---|---|
| grade | Characteristics |
| Grade 1 | Mild; No infusion interruption; No intervention |
| Grade 2 | Infusion interruption indicated but responds promptly to |
| | symptomatic treatment (e.g., antihistamines, NSAIDS, narcotics, |
| | IV fluids); prophylactic medications indicated for <=24 hrs |
| Grade 3 | Prolonged (e.g., not rapidly responsive to symptomatic |
| | medications and/or brief interruption of infusion); recurrence |
| | of symptoms following initial improvement; hospitalization |
| | indicated for clinical sequelae (e.g., renal impairment, |
| | pulmonary infiltrates) |
| Grade 4 | Life threatening consequences; pressor or ventilator support |

TABLE 41
NCI CRS grading scale

| CRS | |
|---|---|
| grade | Characteristics |
| Grade | Symptoms are not life threatening and require symptomatic |
| 1 | treatment only; e.g., fever, nausea, fatigue, headache, myalgias, |
| | malaise |
| Grade | Symptoms require and respond to moderate intervention; |
| 2 | Oxygen requirement <40% or hypotension responsive to fluids |
| | or low dose pressors or Grade 2 organ toxicity |
| Grade | Symptoms require and respond to aggressive intervention; |
| 3 | Oxygen requirement >=40% or Hypotension requiring high dose |
| | or multiple pressors or grade 3 organ toxicity or grade 4 |
| | transaminitis |
| Grade | Life threatening symptoms Requirement for ventilator support or |
| 4 | Grade 4; organ toxicity (excluding transaminitis) |

### Example 1. Humanization of α-TRBV6-5 Antibody Clone Antibody A

The germline for the mouse α-TCRβ antibody clone Antibody A VH and VL were assigned using IMGT nomenclature, with CDR regions defined by a combined Kabat and Chothia classification. SEQ ID NO: 1 and SEQ ID NO: 2 are the Antibody A VH and VL sequences respectively where the VH germline is mouse IGHV1S12*01 and the VL germline is mouse IGKV6-15*01. SEQ ID NOs: 3-5 are the Antibody A VH CDR regions 1-3 respectively and SEQ ID NOs: 6-8 correspond to the VL CDR regions 1-3 (as described in TABLE 30).

Humanization of the Antibody A VH and VL sequences was done separately using similar methodology. Amino acids positions were identified in the framework regions which were important for the success of CDR grafting. Human germline sequences were identified which preserved the necessary residues and contained a high amount of overall identity. When the human germline framework sequence did not contain a matching important amino acid, it was back mutated to match the mouse sequence. CDR regions were grafted onto the human germline unchanged. The Antibody A VH was humanized into human IGHV1-69*01 and the Antibody A VL was humanized into IGKV1-17*01 and IGKV1-27*01. All 3 humanized sequences were confirmed to contain no introduced potential negative post translational modification sites such as NG, DG, NS, NN, DS, NT, NXS, or NXT as a result of the humanization process. SEQ ID NO: 9 is the humanized Antibody A-H.1 VH and SEQ ID NOs: 10 and 11 are the humanized VL IGKV1-17*01 and IGKV1-27*01 germlines respectively (as described in TABLE 30). FIGS. 1A and 1B show the murine and humanized sequences with annotations depicting the CDR and framework regions (FR).

### Example 2: Humanization of α-TRBV12-3 and TRBV12-4 Antibody Clone Antibody B

The germline for the mouse α-TCRβ antibody clone Antibody B VH and VL were assigned using IMGT nomenclature, with CDR regions defined by a combined Kabat and Chothia classification. SEQ ID NO: 15 and SEQ ID NO: 16 are the Antibody B VH and VL sequences respectively where the VH germline is mouse IGHV5-17*02 and the VL germline is mouse IGKV4-50*01. SEQ ID NOs: 17-19 are the B-H VH CDR regions 1-3 respectively and SEQ ID NOs: 20-22 are the B-H VL CDR regions 1-3 (as described in Table 31).

The method applied to humanize Antibody A described in Example 1 was used to humanize Antibody B. The Antibody B VH was humanized into human IGHV3-30*01, IGHV3-48*01, and IGHV3-66*01 and the Antibody B VL was humanized into human IGKV1-9*01, IGKV1-39*01, IGKV3-15*01, IGLV1-47*01 and IGLV3-10*01. SEQ ID NOs: 23-25 are the B-H.1A, B-H.1B, and B-H.1C humanized heavy chains and SEQ ID NOs: 26-30 are the B-H.1D, B-H.1E, B-H.1F, B-H.1G and B-H.1H humanized light chains (as described in Table 31). FIGS. 2A and 2B show the murine and humanized sequences with annotations depicting the CDR and framework regions (FR).

### Example 3: Characteristics of Anti-TCRβV Antibodies

Introduction

Current bispecific constructs designed to redirect T cells to promote tumor cell lysis for cancer immunotherapy typically utilize single chain variable fragments (scFvs) that are derived from monoclonal antibodies (mAb) directed against the CD3e subunit of the T cell receptor (TCR). However, there are limitations to this approach which may prevent the full realization of the therapeutic potential for such bispecific constructs. Previous studies have shown that, e.g., low “activating” doses of anti-CD3e mAb can cause long-term T cell dysfunction and exert immunosuppressive effects. In addition, anti-CD3e mAbs bind to all T cells and thus activate equally all T cells, which has been associated with the first dose side effects of anti-CD3e mAbs that result from massive T cell activation. These large number of activated T cells secrete substantial amounts of cytokines, the most important of which is Interferon gamma (IFNg). This excess amount of IFNg in turn, e.g., activates macrophages which then can overproduce proinflammatory cytokines such as IL-1, IL-6 and TNF-alpha, causing a “cytokine storm” known as the cytokine release syndrome (CRS). Thus, it might be advantageous to develop antibodies that are capable of binding and activating only a subset of necessary effector T cells to reduce the CRS.

Results

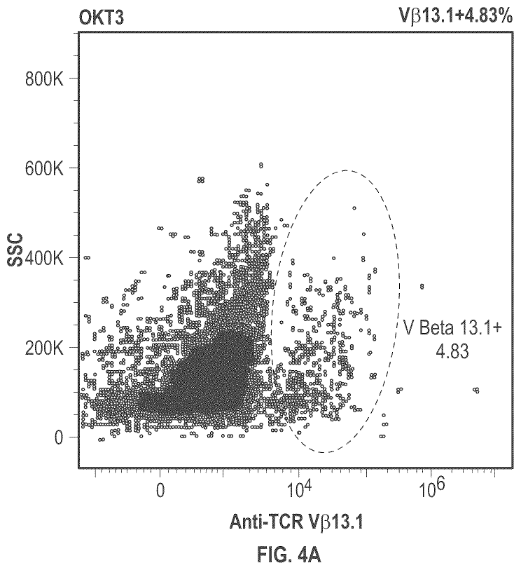

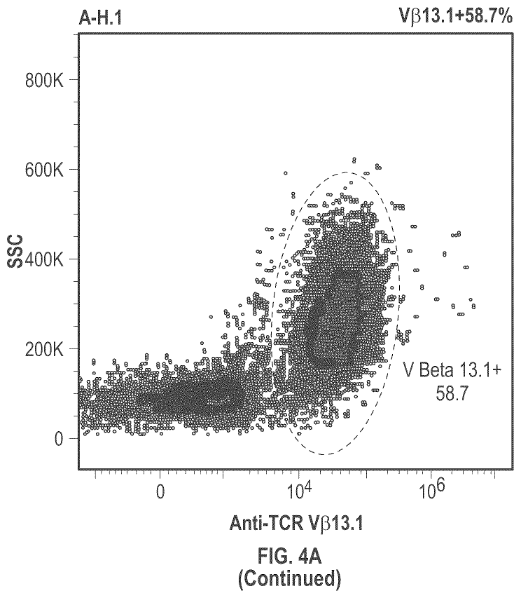

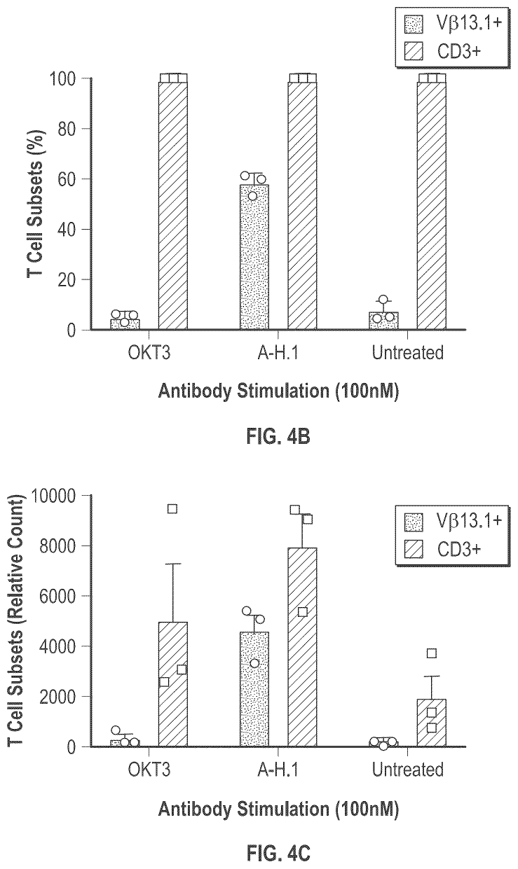

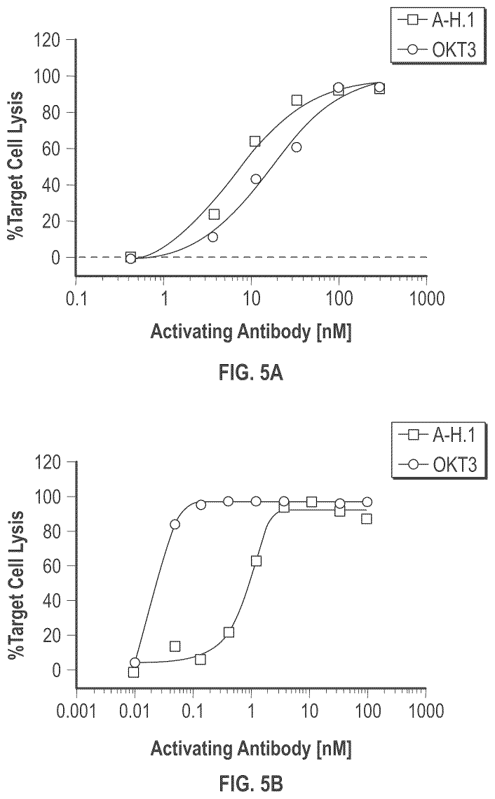

To that end, antibodies directed to the variable chain of the beta subunit of TCR (TCR Vb) were identified. These anti-TCR Vb antibodies bind and activate a subset of T cells, but with, e.g., no or markedly reduced CRS. Using plate-bound anti-TCR Vb13.1 mAbs (A-H.1 and A-H.2) it was shown that a population of T cells, defined by positive staining with A-H.1, can be expanded (from ˜5% of T cells on day 0 to almost 60% of total T cells on day 6 of cell culture) (FIGS. 4A-4C). For this experiment, human CD3+ T cells were isolated using magnetic-bead separation (negative selection) and activated with immobilized (plate-coated) A-H.1 or OKT3 (anti-CD3e) antibodies at 100 nM for 6 days. The expanded Vb13.1+ T cells display cytolytic activity against transformed cell line RPMI-8226 when co-cultured with purified CD3+ T cells (FIGS. 5A-5B).

Next, the ability of PBMCs activated by anti-TCR VB antibodies to produce cytokines was assessed. The cytokine production of PBMCs activated with anti-TCR VB antibodies was compared to the cytokine production of PBMCs activated with: (i) anti-CD3e antibodies (OKT3 or SP34-2); (ii) anti-TCR V alpha (TCR VA) antibodies including anti-TCR VA 12.1 antibody 6D6.6, anti-TCR VA24JA18 antibody 6B11; (iii) anti-TCR alpha beta antibody T10B9; and/or (iv) isotype control (BGM0109). The anti-TCR VB antibodies tested include: humanized anti-TCRVB 13.1 antibodies (A-H.1, or A-H.2), murine anti-TCR VB5 antibody E, murine anti-TCR VB8.1 antibody B, and murine anti-TCR VB12 antibody D. BGM0109 comprises the amino acid sequence of

(SEQ ID NO: 3282)
METDTLLLWVLLLWVPGSTGGLNDIFEAQKIEWHEGGGGSEPRTDTDTC
PNPPDPCPTCPTPDLLGGPSVFIFPPKPKDVLMISLTPKITCVVVDVSE
EEPDVQFNWYVNNVEDKTAQTETRQRQYNSTYRVVSVLPIKHQDWMSGK
VFKCKVNNNALPSPIEKTISKPRGQVRVPQIYTFPPPIEQTVKKDVSVT
CLVTGFLPQDIHVEWESNGQPQPEQNYKNTQPVLDSDGSYFLYSKLNVP
KSRWDQGDSFTCSVIHEALHNHHMTKTISRSLGNGGGGS.

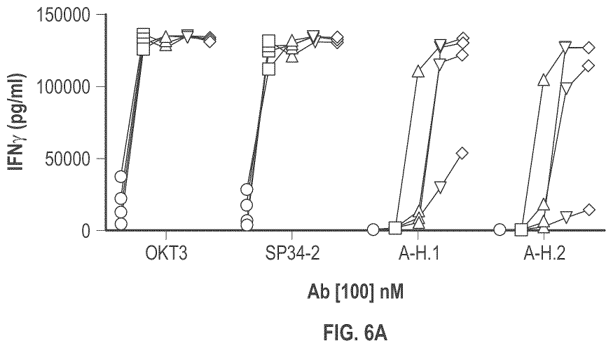

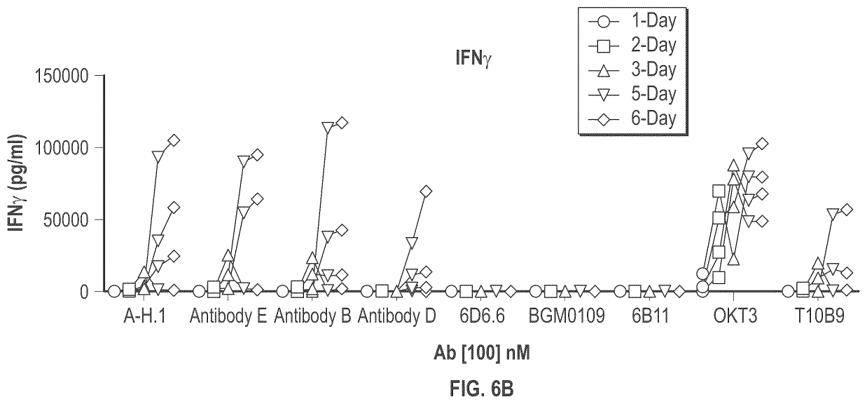

As shown in FIG. 6A, when plate-bound A-H.1 or A-H.2, or anti-CD3e antibodies (OKT3 or SP34-2) were used to activate human PBMCs, the T cell cytokine IFNg was induced (FIG. 6A). All anti-TCR VB antibodies tested had a similar effect on the production of IFNg (FIG. 6B). The anti-TCR VA antibodies did not induce similar IFNg production.

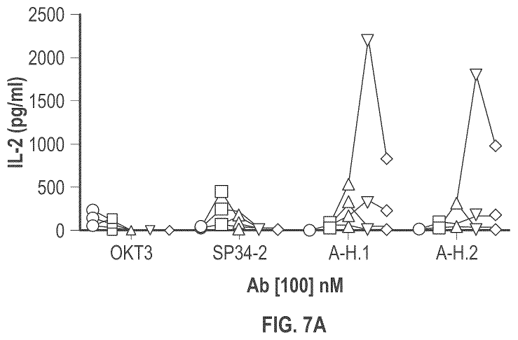

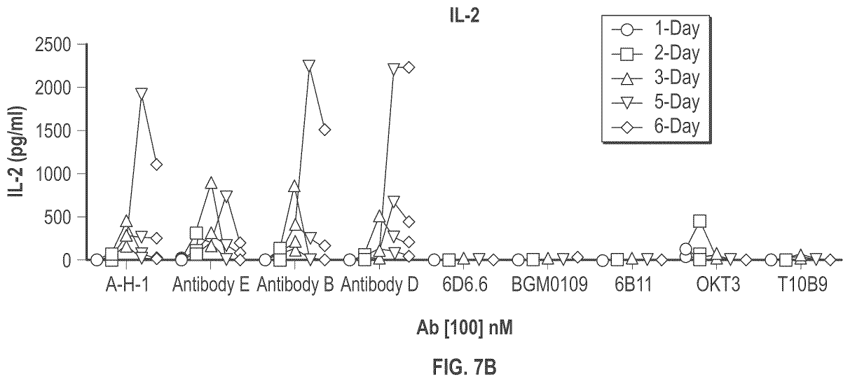

With respect to IL-2 production, PBMCs activated with A-H.1 and A-H.2 resulted in increased IL-2 production (FIG. 7A) with delayed kinetics (FIG. 7B) as compared to PBMCs activated with anti-CD3e antibodies (OKT3 or SP34-2). FIG. 7B shows that anti-TCR VB antibody activated PBMCs demonstrate peak production of IL-2 at Day 5 or Day 6 post-activation (incubation with plate-coated antibodies). In contrast, IL-2 production in PBMCs activated with OKT3 peaked at day 2 post-activation. As with IFNG, the IL-2 effect (e.g., enhanced production of IL-2 and delayed kinetics) was similar across all anti-TCR VB antibodies tested (FIG. 7B).

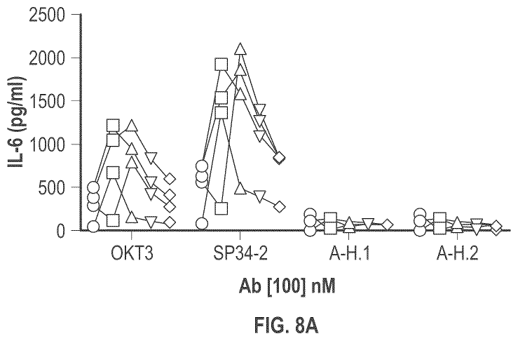

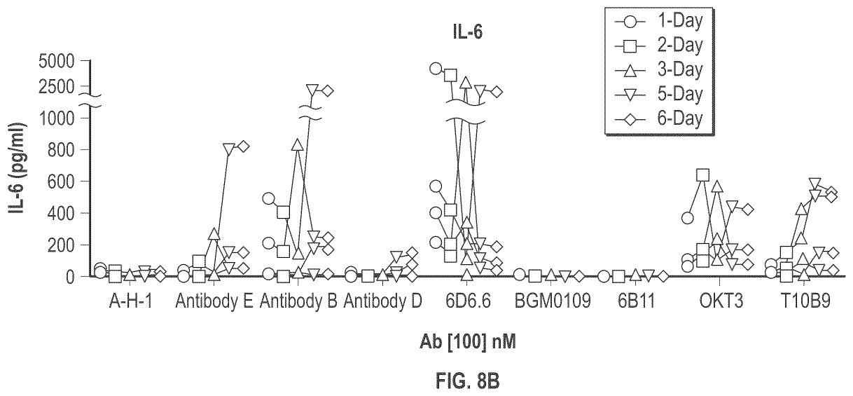

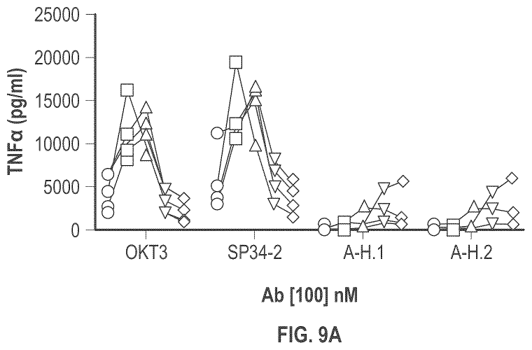

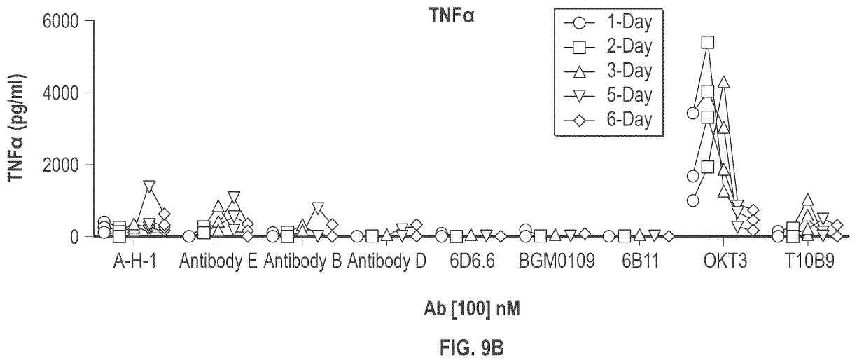

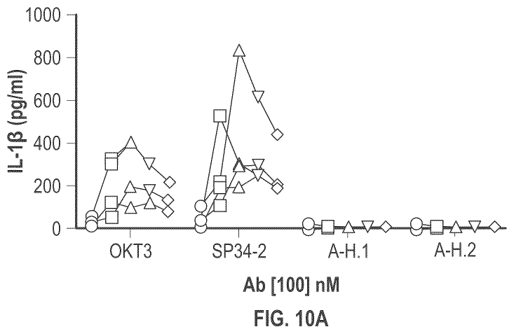

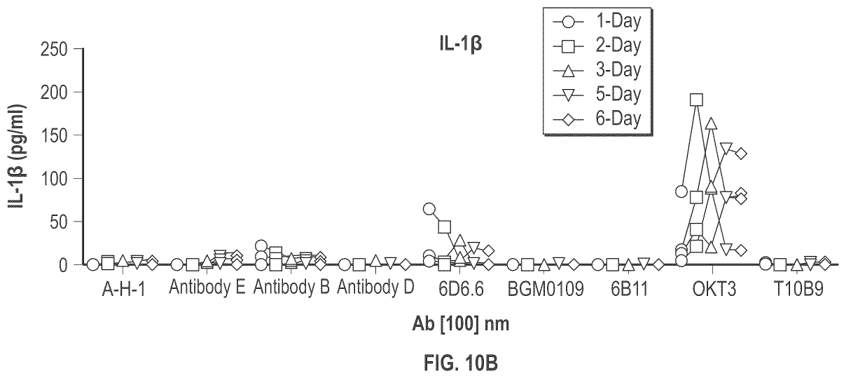

The production of cytokines IL-6, IL-1β and TNF-alpha which are associated with “cytokine storms” (and accordingly CRS) was also assessed under similar conditions. FIGS. 8A, 9A and 10A shows that while PBMCs activated with anti-CD3e antibodies demonstrate production of IL-6 (FIG. 8A), TNF-alpha (FIG. 9A) and IL-1β (FIG. 10A), no or little induction of these cytokines was observed with PBMCs activated with A-H.1 or A-H.2. As shown in FIGS. 9B and 10B, TNF-alpha and IL-1β production was not induced by activation of PBMCs with any of the anti-TCR VB antibodies.

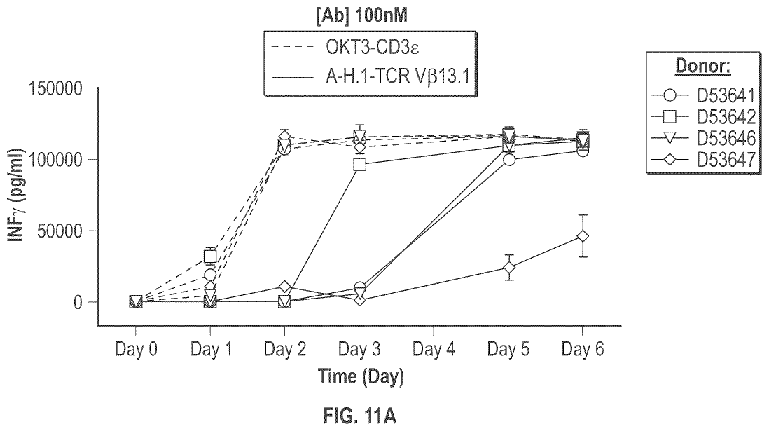

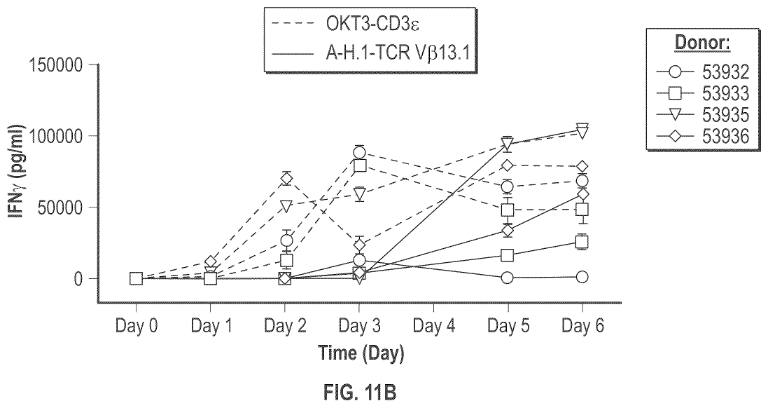

It was further noted that the kinetics of IFNg production by A-H.1-activated CD3+ T cells was delayed relative to those produced by CD3+ T cells activated by anti-CD3e mAbs (OKT3 and SP34-2) (FIGS. 11A and 11B).

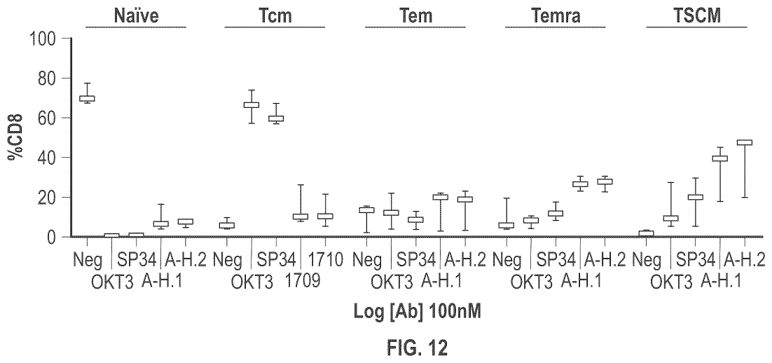

Finally, it was observed that the subset of memory effector T cells known as T_{EM}RA was preferentially expanded in CD8+ T cells activated by A-H.1 or A-H.2 (FIG. 12). Isolated human PBMCs were activated with immobilized (plate-coated) anti-CD3e or anti-TCR Vβ13.1 at 100 nM for 6-days. After a 6-day incubation, T-cell subsets were identified by FACS staining for surface markers for Naive T cell (CD8+, CD95-, CD45RA+, CCR7+), T stem cell memory (TSCM; CD8+, CD95+, CD45RA+, CCR7+), T central memory (Tcm; CD8+, CD95+, CD45RA-, CCR7+), T effector memory (Tem; CD8+, CD95+, CD45RA-, CCR7-), and T effector memory re-expressing CD45RA (Temra; CD8+, CD95+, CD45RA+, CCR7-). Human PBMCs activated by anti-TCR Vβ 13.1 antibodies (A-H.1 or A-H.2) increased CD8+TSCM and Temra T cell subsets when compared to PBMCs activated by anti-CD3e antibodies (OKT3 or SP34-2). Similar expansion was observed with CD4+ T cells.

### CONCLUSION

The data provided in this Example show that antibodies directed against TCR Vb can, e.g., preferentially activate a subset of T cells, leading to an expansion of T_{EMRA}, which can, e.g., promote tumor cell lysis but not CRS. Thus, bispecific constructs utilizing either a Fab or scFv or a peptide directed to the TCR Vb can, e.g., be used to activate and redirect T cells to promote tumor cell lysis for cancer immunotherapy, without, e.g., the harmful side-effects of CRS associated with anti-CD3e targeting.

### Example 4: On-Target T Cell Mediated Cytotoxicity of Multiple Myeloma (MM) Cells with a Dual-Targeting Antibody Molecule Against BCMA and a T Cell Engager

This example shows on-target T cell mediated cytotoxicity of multiple myeloma (MM) cells with dual-targeting antibody molecules that recognize a T cell engager, e.g., TCRVb, on T cells and BCMA on MM cells.

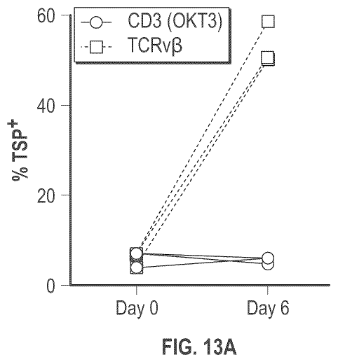

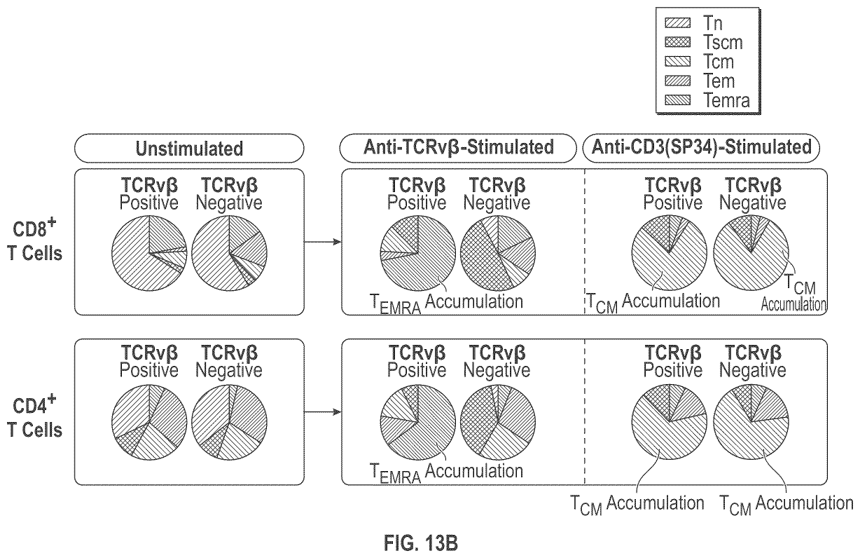

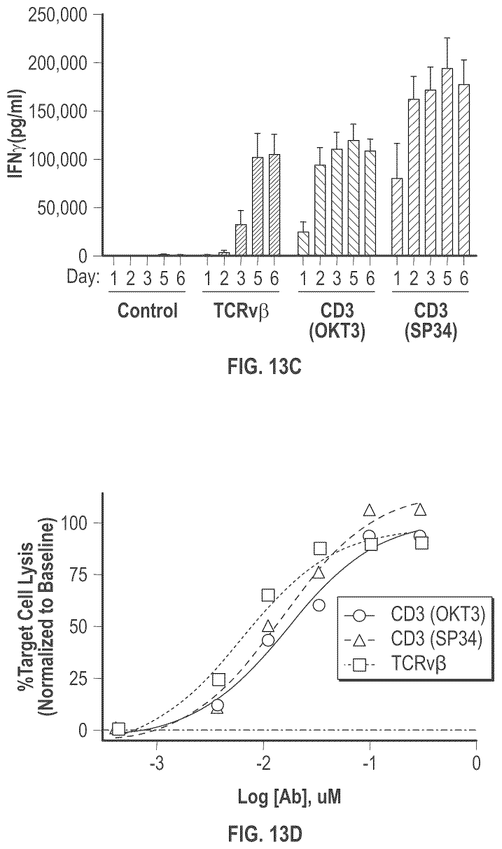

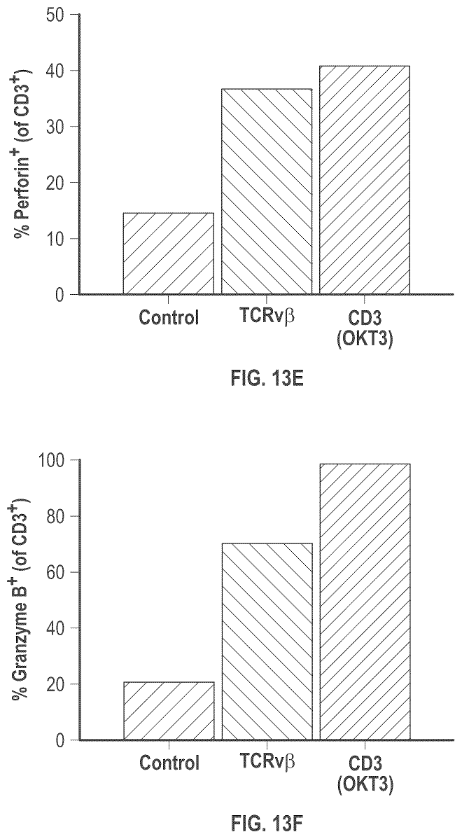

As shown in FIG. 13A, purified human T cells activated with plate-bound anti-TCRVb antibody for 5 days proliferate at a higher rate than purified human T cells activated with plate-bound anti-CD3 (OKT3) antibody. Anti-TCRVb antibody stimulation of T cells resulted in selective expansion of CD45RA+ effector memory CD8+ and CD4+ T cells (TEMRA) cells (FIG. 13B). Both CD8+ and CD4+ Temra cell populations expanded more when stimulated with an anti-TCRVb antibody, compared to unstimulated cells or cells stimulated with an anti-CD3 (SP34) antibody. Anti-TCRVb antibodies resulted in delayed secretion of IFN-g by PBMCs stimulated with an anti-TCRVb antibody compared to PBMCs stimulated with anti-CD3 antibodies (FIG. 13C). Additionally, T cells stimulated with anti-TCRVb antibody or anti-CD3 antibodies resulted in comparable lysis of multiple myeloma target cells, as shown in FIG. 13D. As shown in FIGS. 13E-13F, T cells stimulated for 5 days with 100 ng/ml plate-bound an anti-TCRVb antibody, or an anti-CD3 antibody secreted perforin and Granzyme B.

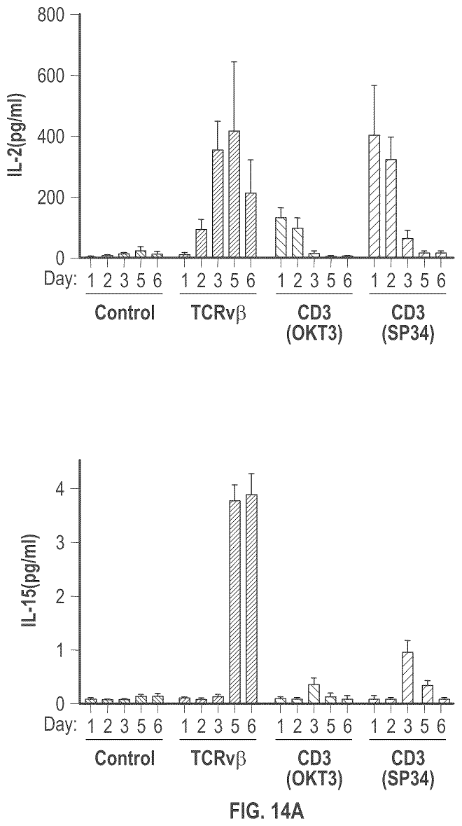

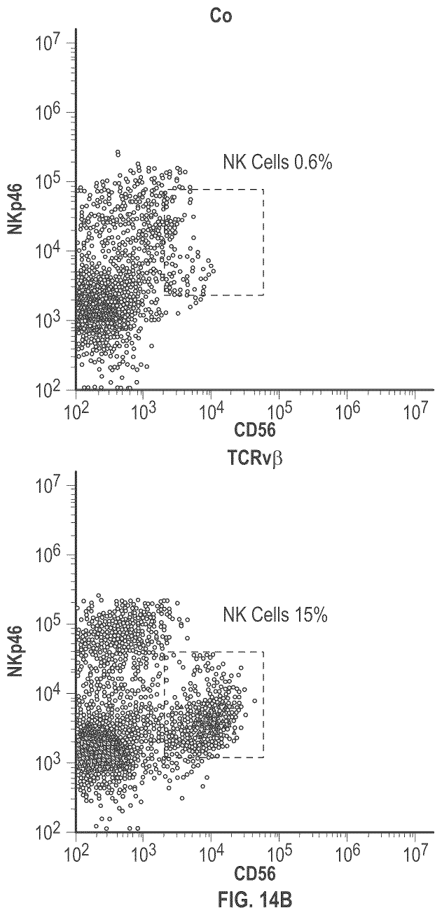

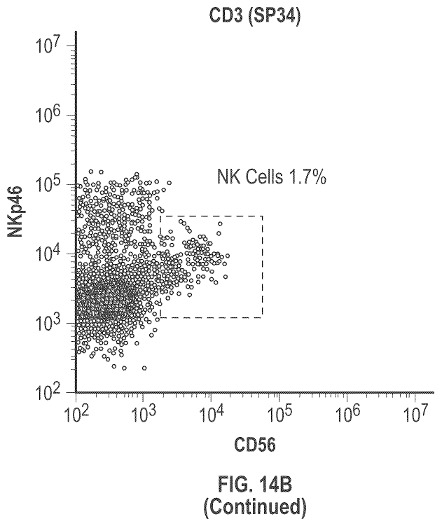

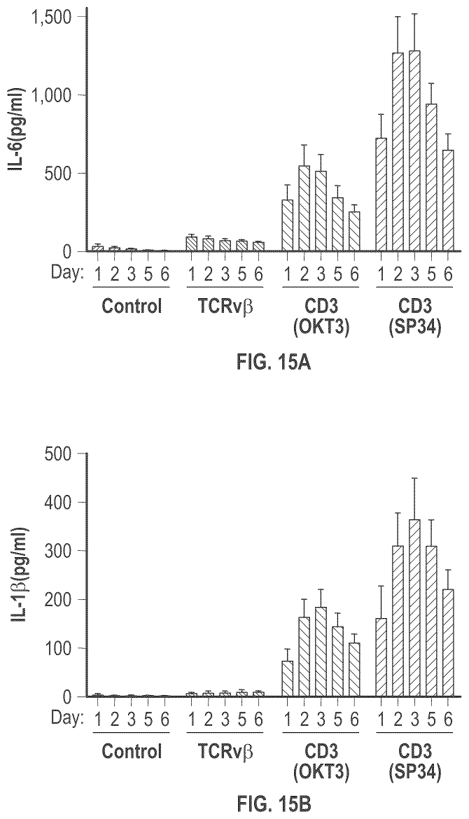

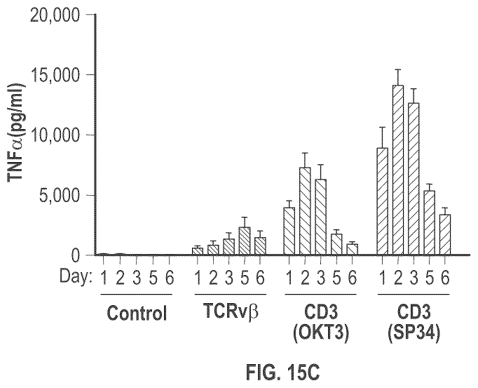

Activation of PBMCs with anti-TCRVb antibody resulted in higher production and/or secretion of IL-2 and/or IL-15 compared to PBMCs activated with an anti-OKT3 antibody (FIG. 14A). Anti-TCRVb antibody activated of PBMCs also resulted in expansion and/or survival, e.g., proliferation of Natural Killer (NK) cells (FIG. 14B). In comparison, PBMCS activated with an anti-OKT3 antibody did not result in NK cell expansion. Further, as described in Example 3, PBMCs activated with an anti-TCRVb antibody did not result in the production of cytokines IL-6, IL-1β and TNF-alpha which are associated with CRS (FIG. 15). These in vitro characterization studies show that in some embodiments, anti-TCRVb antibodies, e.g., activate and/or stimulate, T cells to promote T cell killing as evidenced by target cell lysis, perforin secretion and granzyme B secretion, and secretion of IFN-g with, e.g., delayed kinetics.

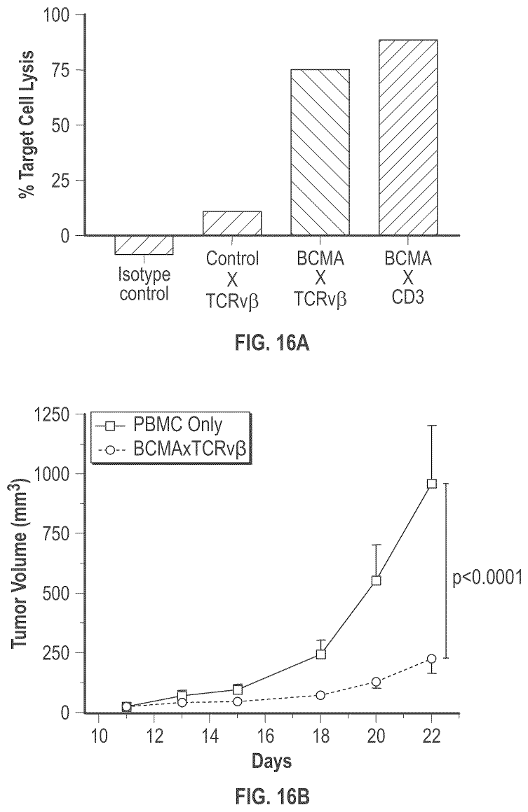

Next, the ability of a dual-targeting antibody molecule, which targets BCMA on one arm and TCRVb on the other arm, to target and kill multiple myeloma (MM) cells was tested. Healthy donor PBMCs were co-incubated with the RMPI8226 MM cell line and one of the following dual-targeting antibody molecules: BCMA-TCRVb, BCMA-CD3, or Control-TCRVb; or an isotype control Target cell lysis was then assessed using flow cytometry. As shown in FIG. 16A, the dual-targeting BCMA-TCRVb antibody molecule resulted in killing of MM cells in vitro.

The dual-targeting BCMA-TCRVb antibody molecule was further tested in vivo for its ability to inhibit MM tumor growth in a MM mouse model. The NCI-H929 cell line was injected in NOD-scid IL2rγnull (NSG)recipient mice on Day 0 followed by delivery of PBMCs on Day 9. On Days 12, 15, 18 and 21, the dual-targeting BCMA-TCRVb antibody molecule was administered via intraperitoneal injection at a dose of 0.5 mg/kg. FIG. 16B shows prevention, e.g., inhibition, of MM tumor growth in vivo with the dual-targeting BCMA-TCRVb antibody molecule. These results demonstrate that in some embodiments the dual-targeting BCMA-TCRVb antibody molecule, e.g., can kill tumor cells, e.g., MM tumor cells, in vitro and in vivo. Accordingly, in some embodiments, a dual-targeting BCMA-TCRVb antibody molecule can be used, e.g., as a therapy for cancer, e.g., a hematological cancer, e.g., MM.

### Example 5: In Vitro Cytotoxicity of a Dual-Targeting Antibody Molecule Against FcRH5 and a T Cell Engager

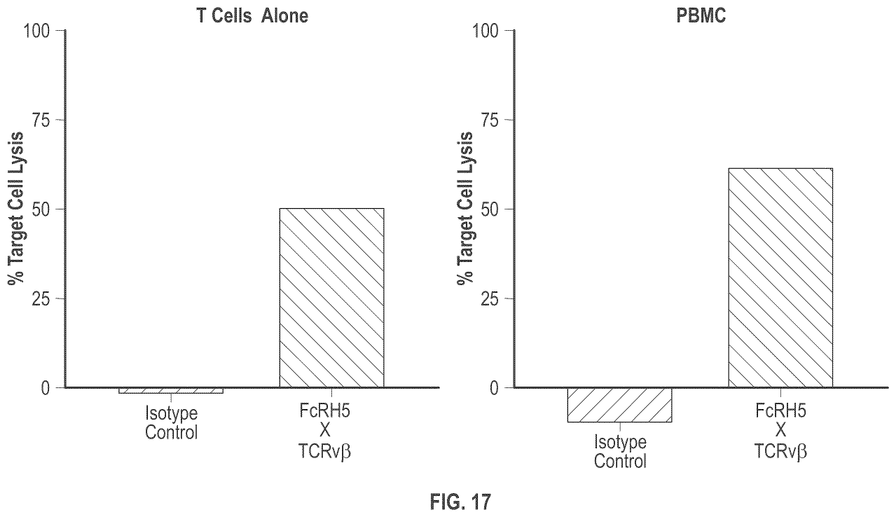

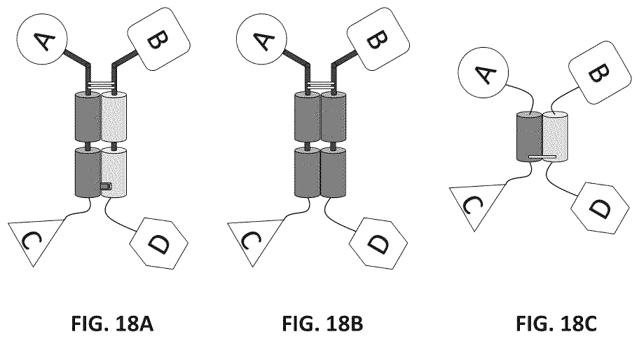

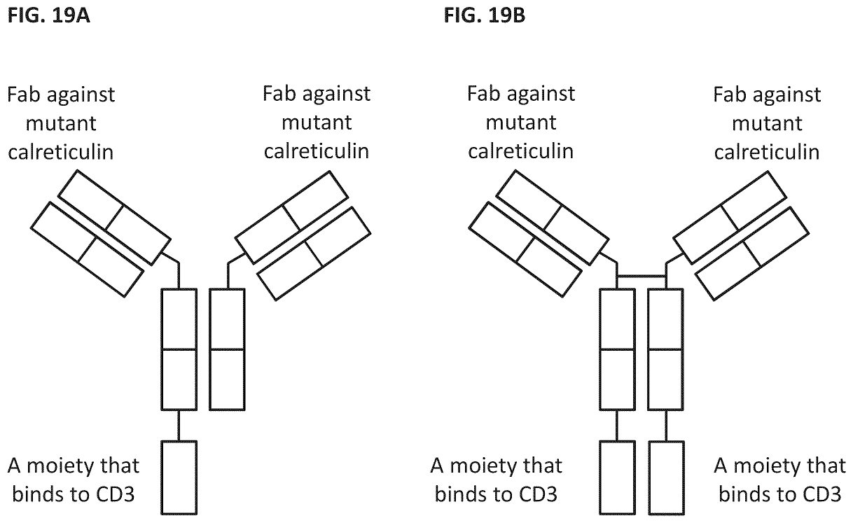

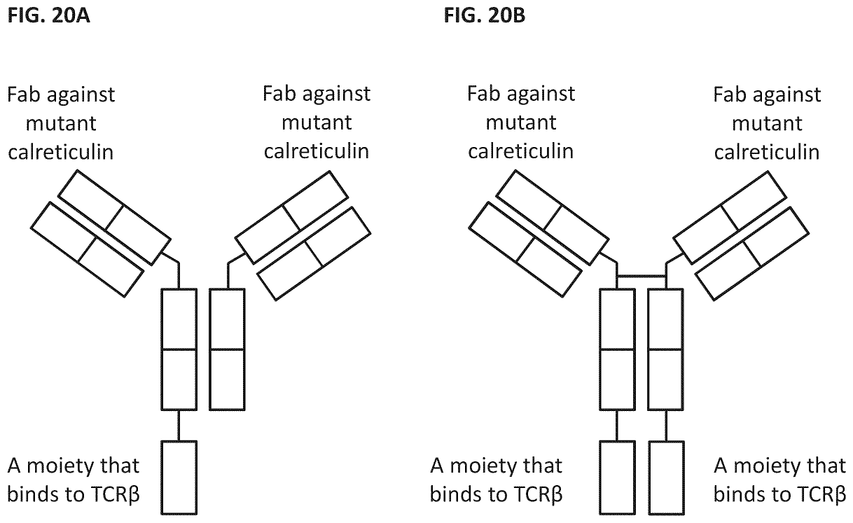

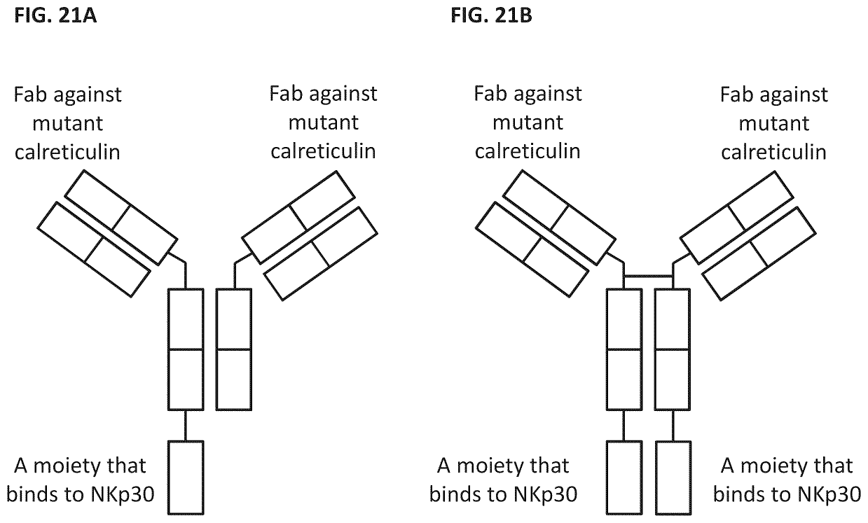

This example shows in vitro cytotoxicity on multiple myeloma (MM) cells with a dual-targeting antibody molecule that recognizes a T cell engager, e.g., TCRVb, on T cells and FcRH5 on MM cells. Healthy donor PBMCs or purified T cells were co-incubated with the MOL8M MM cell line and a dual-targeting antibody molecule which targets FcRH5 on one arm and TCRVb on the other arm, or with an isotype control antibody. Target cell lysis was then assessed using flow cytometry. As shown in FIG. 17, the dual targeting FcRH5-TCRVb molecule resulted in killing of MM cells by both purified T cells or PBMCs. This shows that the dual targeting FcRH5-TCRVb molecule can target and promote killing of MM cells by immune cells, e.g., in PBMCs, including T cells.

### Example 6: Immunization of Armenian Hamster to Generate Anti-NKp30 Antibodies

Briefly, Armenian hamsters were immunized with the extracellular domain of human NKp30 protein in complete Freund's adjuvant and boosted twice on day 14 and day 28 with NKp30 in incomplete Freund's adjuvant (IFA). On day 56 one more boost in IFA was given and the animals harvested three days later. Spleens were collected and fused with P3X63Ag8.653 murine myeloma cell line. 0.9×10\hat{ }5 cells/well in 125 μl were seated in 96 well plate and feed with 125 μl of I-20+2ME+ HAT (IMDM (4 g/L glucose) supplemented with 20% fetal bovine serum, 4 mM L-glutamine, 1 mM sodium pyruvate, 50 U penicillin, 50 μg streptomycin and 50 μM 2-ME in the absence or presence of HAT or HT for selection, and Hybridoma Cloning Factor (1% final) on days 7, 11 and thereafter as needed. At approximately 2 weeks after fusion (cells are about 50% confluent) supernatant was collected and assayed for binding.

### Example 7: Hybridoma Screen for NKp30 mAbs

Expi293 cells were transfected with BG160 (hNKp30 cell antigen) 18 hours prior to screening. The day of screening, transfected cells were diluted to 0.05×10\hat{ }⁶/mL and anti-Armenian hamster Fc Alexa Fluor 488 added to a final concentration of 0.4 ug/mL. 50 uL (2,500 cells) of this mixture was added to each well of a 384 well plate. The same density of untransfected 293 cells with secondary were used as a negative control. 5 uL of hybridoma supernatant was added to the cell mixture and the plate incubated for 1 hour at 37° C. The plates were then imaged on Mirrorball. Positive clones were identified and subcloned by serial dilution to obtain clonal selected hybridoma. After reconfirmation using the same protocols the hybridoma cells were harvested and the corresponding heavy and light chain sequences recovered. The DNA was subcloned into pcDNA3.4 for subsequent expression of the corresponding antibodies and further validation.

### Example 8: Binding of NKp30 Antibodies to NK92 Cells

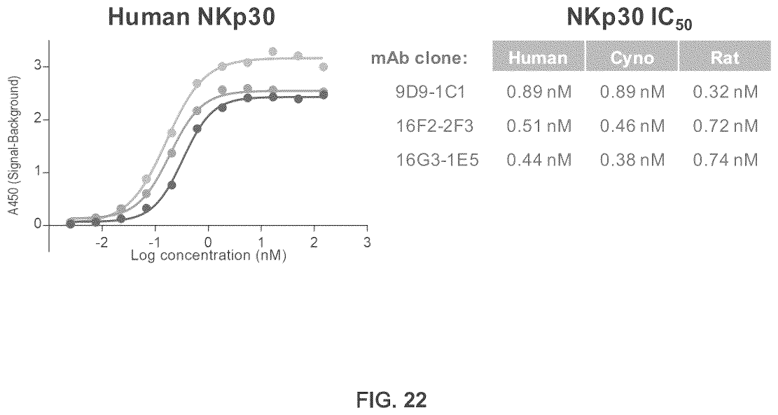

NK-92 cells were washed with PBS containing 0.5% BSA and 0.1% sodium azide (staining buffer) and added to 96-well V-bottom plates with 200,000 cells/well. Hamster NKp30 antibodies were added to the cells in 2.0 fold serial dilutions and incubated for 1 hour at room temperature. The plates were washed twice with staining buffer. The secondary antibody against hamster Fc conjugated to AF647 (Jackson, 127-605-160) was added at 1:100 dilution (1.4 mg/ml stock) and incubated with the cells for 30 minutes at 4° C. followed by washing with staining buffer. Cells were subsequently were fixed for 10 minutes with 4% paraformaldehyde at room temperature. The plates were read on CytoFLEX LS (Beckman Coulter). Data was calculated as the percent-AF747 positive population (FIG. 22).

### Example 9: Bioassay to Measure Activity of NKp30 Antibodies Using NK92 Cell Line

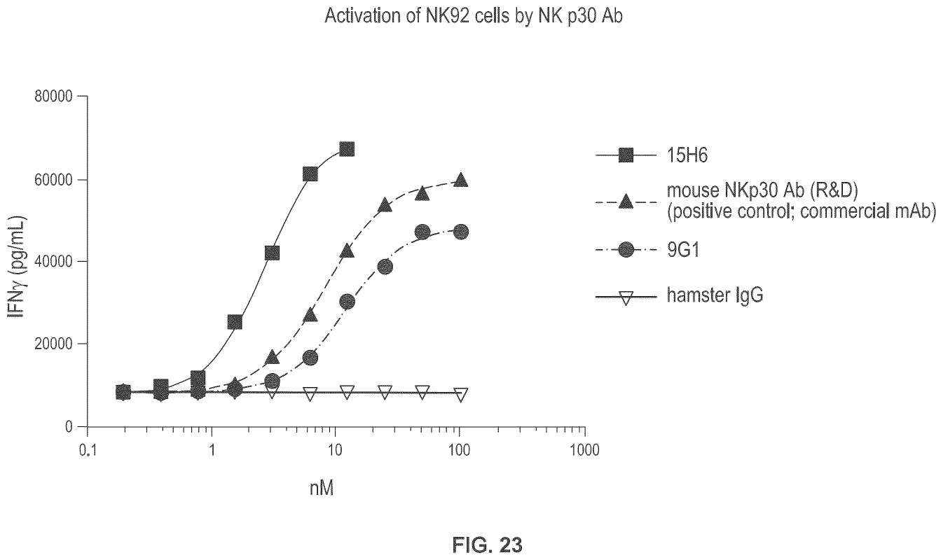

NKp30 antibodies were three-fold serially diluted in PBS and incubated at 2-8° C. overnight in flat bottom 96 well plates. Plates were washed twice in PBS and 40,000 NK-92 cells were added in growth medium containing IL-2. Plates were incubated at 37° C., 5% CO2, humidified incubator for 16-24 hours before supernatants were collected. IFNγ levels in supernatants was measured following MSD assay instructions (FIG. 23). Supernatant collected from cells incubated with hamster isotype IgG was used as negative control and supernatants from cells incubated with NKp30 monoclonal antibody (R&D, clone 210847) was utilized as a positive control. Data were generated using hamster anti-NKp30 mABs.

### Example 10: Characterization of Anti-Calreticulin Antibodies

A murine anti-calreticulin antibody AbM-1 (also referred to as BIM0031) which comprises a VH of SEQ ID NO: 6250 and a VL of SEQ ID NO: 6252 was humanized. Five humanized VHs (SEQ ID NOs: 6372 and 234-237 shown in Table 16) and five humanized VLs (SEQ ID NOs: 238-242 shown in Table 16) were generated. All the humanized VHs comprise a cysteine to alanine substitution in HCDR2. Antibodies BJM0040-BJM0064, as disclosed in Table 19, were synthesized and characterized for their biochemical and functional activities.

Briefly, expression level of purified proteins was measured after protein A elution. Proteins were analyzed by analytical SEC to assess aggregation and tested by differential scanning fluorimetry (DSF) to identify more stable cantidates. Binding affinity of the cantidates was measured in ELISA assay against mutant calreticulin C-terminal peptide fused to a human Fc. The results were summarized in Table 22. Humanized antibodies comprising the cysteine to alanine substitution in HCDR2 demonstrated reduced aggregation compared to the parental murine antibody.

TABLE 22
Summary of characterization of anti-calreticulin antibodies

| | yield | % aggregation | Tm | ELISA |
|---|---|---|---|---|
| | (mg/L) | after ProA | (C.) | IC50 |
| BJM0040 | 95.7 | 0 | 75 | 12.34 |
| BJM0041 | 193.6 | 5.3 | 75 | 18.79 |
| BJM0042 | 106.7 | 0 | 75 | 10.52 |
| BJM0043 | 181.5 | 3 | 75 | 8.279 |
| BJM0044 | 161.7 | 5.6 | 75 | 16.19 |
| BJM0045 | 42.9 | 8 | 73 | ~836101 |
| BJM0046 | 116.6 | 7.5 | 76 | ~362165 |
| BJM0047 | 93.5 | 7 | 76 | 802.9 |
| BJM0048 | 111.1 | 6 | 75 | 430.3 |
| BJM0049 | 103.4 | 6.8 | 76 | 943.6 |
| BJM0050 | 261.8 | 10.3 | — | 597627 |
| BJM0051 | 112.2 | 7.4 | 77 | 780.7 |
| BJM0052 | 123.2 | 12.4 | 77 | 776.2 |
| BJM0053 | 132 | 10.3 | 76 | 357.2 |
| BJM0054 | 128.7 | 12.3 | 77 | 657.2 |
| BJM0055 | 72.6 | 17.1 | 69 | 1E+06 |
| BJM0056 | 113.3 | 11.6 | 69 | 889.5 |
| BJM0057 | 67.1 | 12.1 | 69 | 4E+06 |
| BJM0058 | 92.4 | 9.1 | 69 | 498.4 |
| BJM0059 | 136.4 | 12 | 68 | 83.11 |
| BJM0060 | 134.2 | 7.5 | 72 | ~4347 |
| BJM0061 | 140.8 | 8.5 | 73 | 356 |
| BJM0062 | 91.3 | 8.5 | 73 | 351.8 |
| BJM0063 | 145.2 | 8.8 | 73 | 988.6 |
| BJM0064 | 139.7 | 10 | 73 | 637.4 |
| BIM0031 | | | | 24.32 |

### Example 11: Generation and Characterization of Humanized Anti-NKp30 Antibodies

A series of hamster anti-NKp30 antibodies were selected. These antibodies were shown to bind to human NKp30 and cynomolgus NKp30 and induce IFNγ production from NK-90 cells (data not shown). The VH and VL sequences of exemplary hamster anti-NKp30 antibodies 15E1, 9G1, 151H6, 9D9, 3A12, and 12D10 are disclosed in Table 9. The VH and VL sequences of exemplary humanized anti-NKp30 antibodies based on 15E1, 9G1, and 151H6 are also disclosed in Table 9. The Kabat CDRs of these antibodies are disclosed in Table 34 and Table 8.

Two humanized constructs based on 15E1 were selected. The first construct BJM0407 is a Fab comprising a heavy chain variable region comprising the amino acid sequence of SEQ ID NO: 7302 and a lambda light chain variable region comprising the amino acid sequence of SEQ ID NO: 7305. Its corresponding scFv construct BJM0859 comprises the amino acid sequence of SEQ ID NO: 7310. The second construct BJM0411 is a Fab comprising a heavy chain variable region comprising the amino acid sequence of SEQ ID NO: 7302 and a kappa light chain variable region comprising the amino acid sequence of SEQ ID NO: 7309. Its corresponding scFv construct BJM0860 comprises the amino acid sequence of SEQ ID NO: 7311. BJM0407 and BJM0411 showed comparable biophysical characteristics, e.g., binding affinity to NKp30 and thermal stability. The scFv constructs BJM0859 and BJM0860 also showed comparable biophysical properties.

### Example 12: Binding of an Exemplary Anti-Calreticulin Antibody Molecule to Wild-Type or Mutant Calreticulin

In this example, an exemplary anti-calreticulin antibody molecule, BKM0106 (parental IgG form of antibody 6C10), was tested for binding to wild-type calreticulin (CALR WT) compared to two calreticulin mutants (CALR ins and CALR del, the sequences of which are listed in Table 2 as SEQ ID Nos: 1002 and 1003, respectively). Two ELISA tests were run, one in which the antigen was coated in the plate, and one in which the antibody was coated on the plate.

For the experiment in which antigen was coated on the plate, a Maxisorp plate was coated with CALR WT, CALR in, or CALR del protein. The plate was blocked with 3% BSA. BKM0106 was diluted to 100 nM and added to the wells. Anti-Human HRP (Jackson Immunoresearch 309-035-008) 1:20,000 was added to each well. 1-Step Turbo TMB-ELISA Substrate solution was added and the reaction was stopped by adding 1M HCl. The plate was read at 450 nm.

For the experiment in which antibody was coated on the plate, a Maxisorp plate was coated with BKM0106. The plate was blocked with 3% BSA. CALR WT, CALR ins, and CALR del were diluted to 100 nM and added to separate wells. Anti-His-Tag-HRP (Southern Biotech 4603-05) 1:5,000 was added to each well. 1-Step Turbo TMB-ELISA Substrate solution was added and the reaction was stopped by adding 1M HCl. The plate was read at 450 nm.

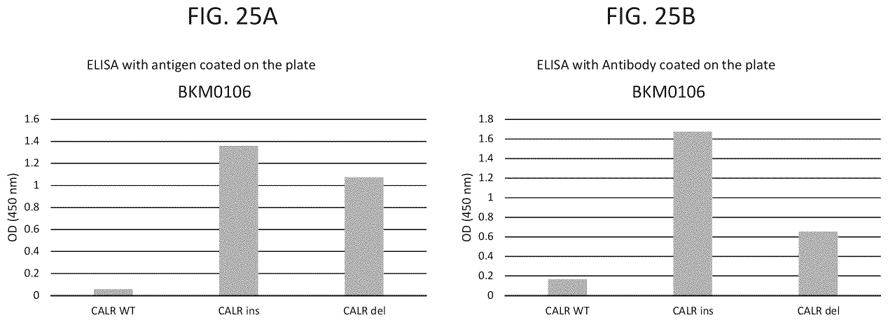

As shown in FIGS. 25A-25B, the exemplary antibody BKM0106 bound to the CALR ins and CALR del mutants, but did not bind to wild-type calreticulin.

### Example 13: Binding of an Exemplary Anti-Calreticulin Antibody Molecule to Cells Expressing Mutant Calreticulin

In this example, an exemplary anti-calreticulin antibody molecule, BKM0106 (parental IgG form of antibody 6C10), was tested for binding to cells expressing mutant calreticulin (CALR ins and CALR del, the sequences of which are listed in Table 2 as SEQ ID Nos: 1002 and 1003, respectively). Briefly, Expi293F cells (Thermo Fisher A14527) were triple transfected with BH470 (human TpoR), BH472 (human JAK2) and either human CALR ins, human CALR del, or BH800 (human CALR WT) cell antigens. BKM0106 and the hIgG1 isotype control at 0.78, 1.56, 3.125, 6.25, 12.5, 25, and 50 nM were added to the wells. Cells were incubated with secondary antibody Alexa Fluor 488 Anti-Human IgG (Jackson ImmunoResearch 109-545-088) 1:200. Zombie Violet BV412 (BioLegend 423114) viability dye was added to the cells 1:100.

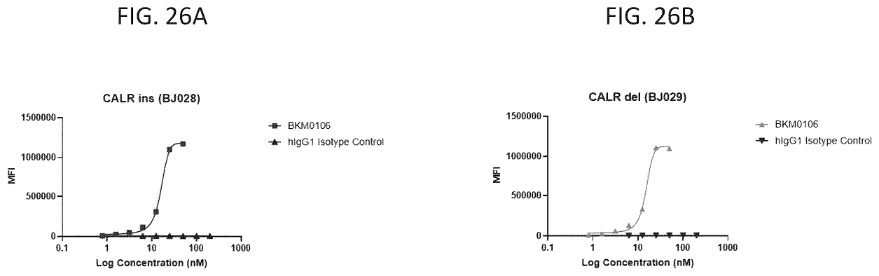

As shown in FIGS. 26A-26B, BKM0106 bound to both CALR ins and CALR del with similar responses.

### Example 14: Targeting mtCALR+ Cells Via Bridging mtCALR+ Cells to Immune Effector Cells Via Bispecific Antibodies

In this example, a series of antibody molecules was tested for therapeutic efficacy in a syngeneic murine cancer model. Briefly, surrogate molecules with a mIgG2a backbone were generated from the exemplary anti-calreticulin antibody molecule 6C10 and each of the following effector arms:
BKM0201-mutCALR-6C10 monoclonal-ADCC enabled
BKM0202-mutCALR-6C10×TCRvB (2×2) bispecific-LALAPG
BKM0204-mutCALR-6C10×CD3-2C11 (1×1) bispecific-LALAPG

The therapeutic efficacy of the above molecules were tested in a systemic murine model generated using Ba/F3 cells engineered cells expressing mtCALR, hMPL, and hJAK2. On day 1 of the study, 2×10\hat{ }6 engineered Ba/F3 mtCALR, hMPL, hJAK2 cells, suspended in PBS, were intravenously injected in to female Balb/C mice. Mice were treated a dose of 1 mg/kg or 5 mg/kg of CALR×/TCRvB and control bispecific molecules every three days for a total of 4 doses (day 4, 7, 10 and 13) by intravenous bolus injection. Animals were monitored for humane endpoints and Body weights were monitored twice weekly. All animals were euthanized on day 15 and spleens were excised, and spleen weights were monitored as a surrogate for disease burden. Data indicates that vehicle treated mice in Ba/F3 mtCALR, hMPL, hJAK2 cell engrafted mice show nearly three-fold increase in spleen weight as compared to the naïve mice suggesting increased disease burden.

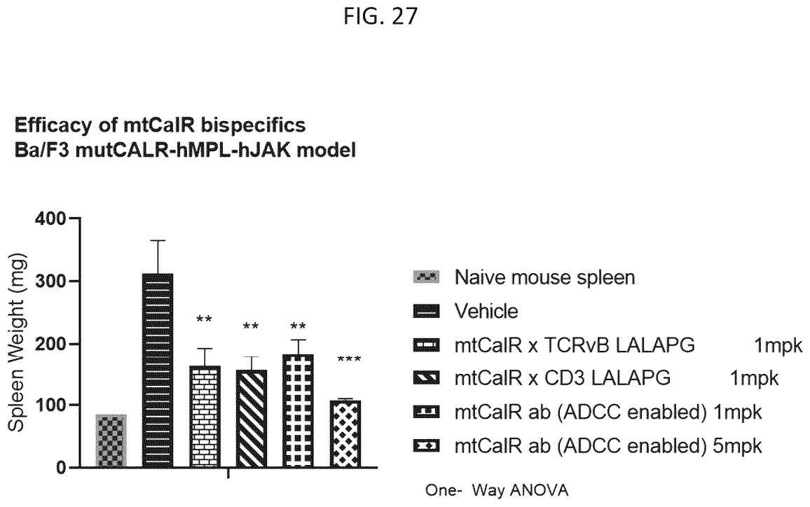

As shown in FIG. 27, all three antibody treated mice showed a significant decrease in spleen weights as compared to vehicle control.

### Example 15: Biacore Analysis of Exemplary Anti-NKp30 Antibody Molecules

In this example, a series of exemplary anti-NKp30 antibody molecules were analyzed for their binding affinity for NKp30. Briefly, surface plasmon resonance (SPR) measurements were performed by using the BIAcore T200. Human NKp30 (BKM0179) was immobilized on a CM5 chip via anti-mouse Fc antibody to a response of 50 RU. Each exemplary antibody construct were injected at concentrations of 3.9, 7.8, 15.6, 31.2, 62.5, and 125 nM, and at a flow rate of 20 μl/min, over the surface on which the human NKp30 was immobilized. The data was fit using a 1:1 binding model.

As shown in Table 23, most of the exemplary antibodies showed preserved affinity to human NKp30 compared to the parental antibody.

TABLE 23
Biacore results

| | | Human Nkp30 | |
|---|---|---|---|
| Construct | Description | (BKM0179) | |
| BJM1078 | BJM0407 Parental | 1.48 | nM |
| BJM1079 | BJM0411 Parental | 1.26 | nM |
| BKM0138 | BJM0411 VL-N95A | 3.2 | nM |
| BKM0139 | BJM0411 VL-D92A | 3.2 | nM |
| BKM0140 | BJM0407 VL-D92A | 3.3 | nM |
| BKM0141 | BJM0407 VL-N95A | 3.0 | nM |
| BKM0142 | BJM0411 VH-N60A | 1.28 | nM |
| BKM0143 | BJM0407 VH-N60A | 1.45 | nM |
| BKM0144 | BJM0411 VH-N60A-VL-D92A-N95A | 6.4 | nM |
| BKM0145 | BJM0407 VH-N60A-VL-D92A-N95A | 4.2 | nM |

### Example 16: Production and Assessment of Exemplary Anti-CD3 Antibody Molecules

Production of Anti-CD3 Antibody Molecules

Four C57/BL6 mice were immunized with KLH conjugated CD3e peptide bearing the sequence QDGNEEMGGITQTPYKVSISGTTVILTC (SEQ ID NO: 7487). Animals were bled three days after fourth immunization to check for titers. After the fourth immunization, animals were boosted twice with the antigen. Three to four days after final boost, animals are sacrificed for spleen or lymph node tissue harvest. Spleen was fused using standard fusion methods which produced 20 plates of hybridoma cells. The primary screen was performed using ELISA by checking binding to the peptide followed by CD3e protein. One clone, 4D4, was selected for expansion and following subcloning protocol, generated monoclonal hybridoma.

Binding of Anti-CD3 Antibody Molecules to CD3 In Vitro

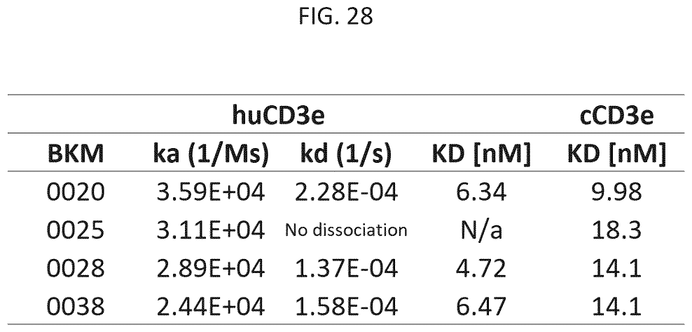

In this example, exemplary humanized anti-CD3 antibody molecules BKM0020, BKM0025, BKM0028, BKM0038 (as described herein) were tested for their binding affinity to human or cynomolgus CD3e using surface plasmon resonance (SPR). SPR measurements were performed by using the BIAcore T200. Each construct was immobilized on a CM5 chip via Anti-human Fc antibody to a response of 200 RU. Human CD3e (Acro Biosystems CDE-H5223) was diluted to 500 nM and then diluted two-fold. Each analyte concentration was injected at a flow rate of 20 μl/min over the surface on which each antibody was immobilized. The data was fit using a 1:1 binding model. Binding affinity results are shown in FIG. 28. Affinity to human CD3e was preserved compared to the parental and affinity to cyno CD3e was two to three-fold lower compared to the parental antibody.

Binding of Anti-CD3 Antibody Molecules to CD3 Expressed on Cells

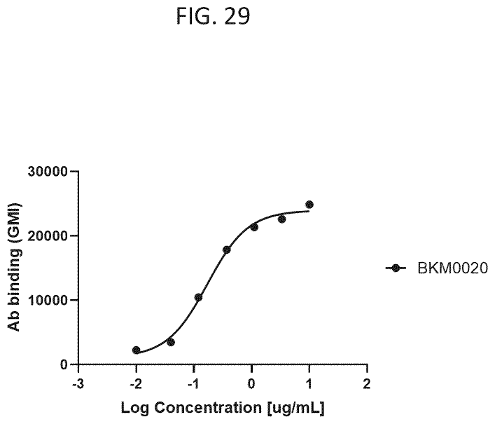

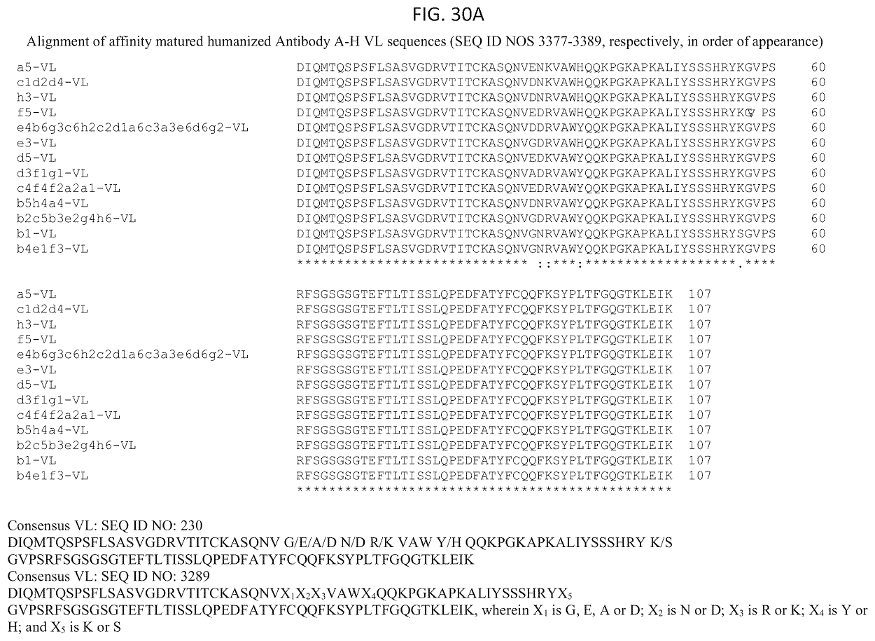

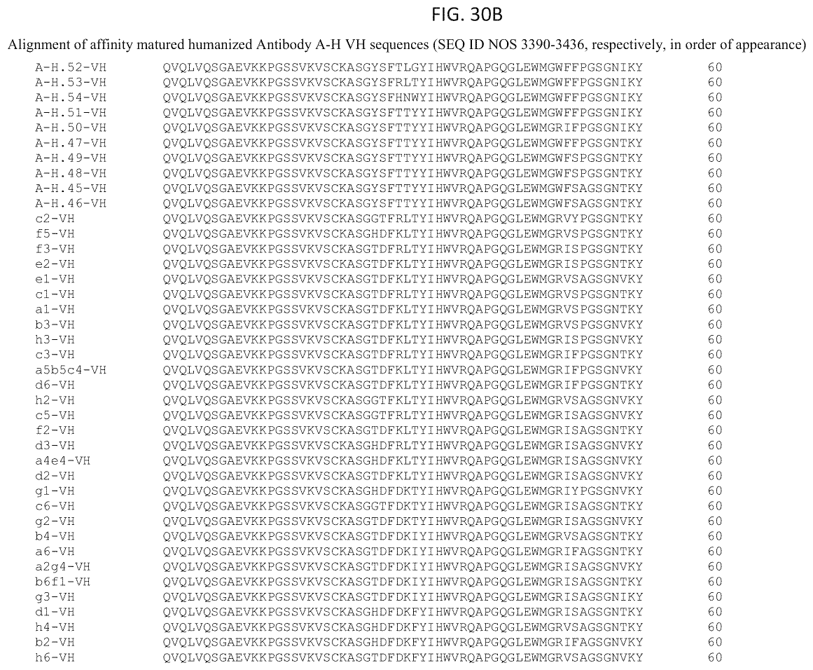

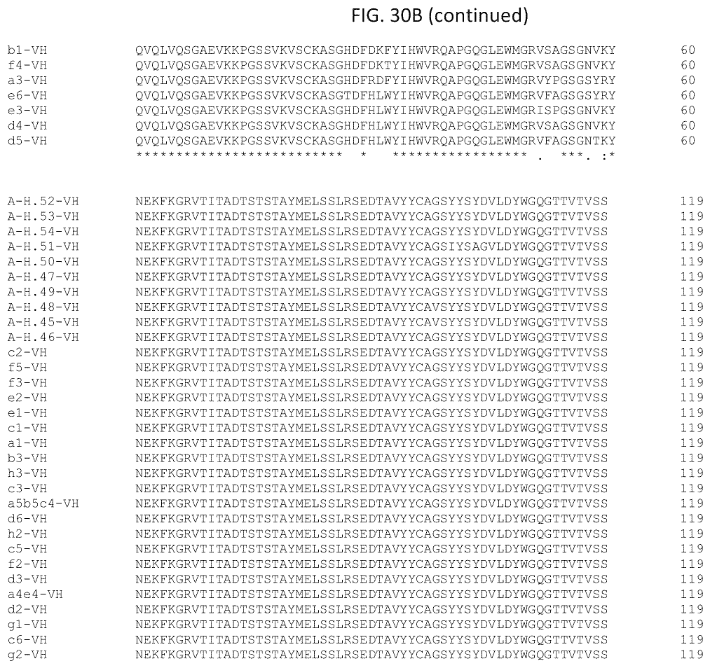

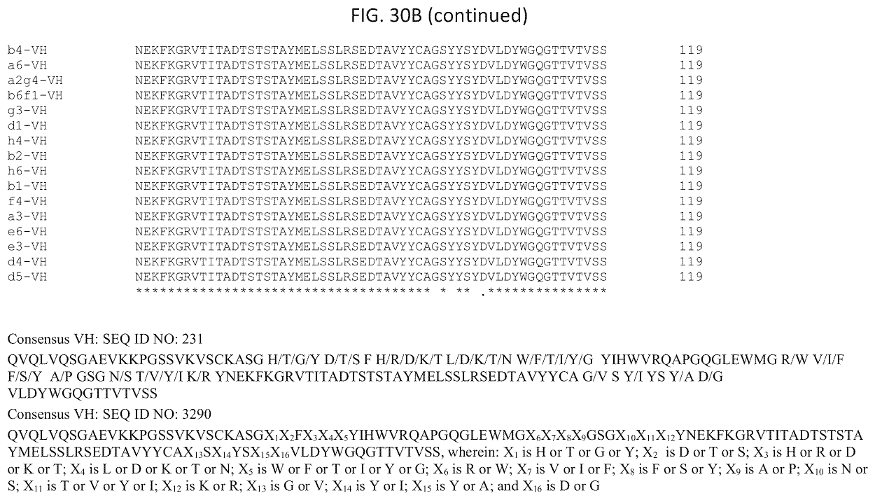

Binding of the exemplary humanized anti-CD3 antibodies to CD3-expressing Jurkat cells was performed using FACS. Antibodies were tested in a series of 3-fold dilutions starting with 10 ug/ml concentration and detected using anti-human IgG secondary antibodies conjugated to AF647. As shown in FIG. 29, humanized anti-CD3 mAb showed strong binding to Jurkat cells expressing CD3.

### Example 17: Generation of Anti-Calreticulin (CALR) Antibodies

Two Armenian Hamsters (86 and 87) were immunized with mutCALR 5 bp ins (BJ028) antigen. Hamster #86 was chosen to perform fusion using standard procedures. Fusion produced 12 hybridoma plates that were screened by ELISA for binding to BJ028 (mut CALR ins), BJ027 (wild-type CALR) and BIM0167 (CALR mutant peptide fused to a human Fc). 15 clones were further selected for expansion and subcloning. Two clones were selected for further characterization and sequencing to obtain V gene sequences. The hamster sequences were further humanized by grafting CDRs on to human frameworks. The resulting humanized mAbs were tested for binding using Biacore and FACS, e.g., as described above.

### Example 18: Optimization of α-TRBV6-5 Antibody

The anti TRBV6-5 antibody was optimized to improve affinity for the human and cyno antigen, improve thermal stability, and remove sequence motifs that might pose chemical stability liabilities. ScFv libraries were built using random mutagenesis (Caldwell et al. (1992) Randomization of genes by PCR mutagenesis. PCR Meth. Appl. 2:28) or a modified version of Kunkel mutagenesis (Kunkel T A. (1985) Rapid and efficient site-specific mutagenesis without phenotypic selection. PNAS 82(2): 488-92). For affinity improvement, library selections vs human and cyno antigens were performed using standard phage display (Lee, C M et al. (2007) Selection of human antibody fragments by phage display. Nature protocols 2, 3001) and yeast display techniques (Chao G, et al. (2006) Isolating and engineering human antibodies using yeast surface display. Nature Protocols. 1(2):755-69). Thermal challenge of phage or yeast populations was used to select for clones with improved thermal stability. Selections were followed by standard screening methods such as ELISA and flow cytometry to identify individual clones with improved properties. Following hit sequencing and analysis of mutation-activity correlation, second-generation libraries were constructed using the same methods above. Library selections and individual clone screening were repeated as above with the modification that more stringent conditions were applied to select for clones with maximized activity. Following hit sequencing, scFv genes were reformatted into the biologically relevant antibody format for expression, purification, and triaging.

### INCORPORATION BY REFERENCE

All publications and patents mentioned herein are hereby incorporated by reference in their entirety as if each individual publication or patent was specifically and individually indicated to be incorporated by reference.

### EQUIVALENTS

Those skilled in the art will recognize, or be able to ascertain using no more than routine experimentation, many equivalents to the specific embodiments of the invention described herein. Such equivalents are intended to be encompassed by the following claims.

### EXEMPLARY EMBODIMENTS

Additional features of any of the aforesaid multifunctional molecules, nucleic acids, vectors, host cells, or methods include one or more of the following exemplary embodiments.

Those skilled in the art will recognize, or be able to ascertain using no more than routine experimentation, many equivalents to the specific embodiments of the invention described herein. Such equivalents are intended to be encompassed by the following exemplary embodiments.

### Exemplary Embodiment 1

The disclosure relates, inter alia, to novel multispecific or multifunctional molecules that include (i) an antigen binding domain that binds to a calreticulin protein (e.g., a wild-type or mutant calreticulin protein); and one, two or all of: (ii) an immune cell engager (e.g., chosen from an NK cell engager, a T cell engager, a B cell engager, a dendritic cell engager, or a macrophage cell engager); (iii) a cytokine molecule; and/or (iv) a stromal modifying moiety. The terms “multispecific” or “multifunctional” are used interchangeably herein.

Without wishing to be bound by theory, the multispecific or multifunctional molecules disclosed herein are expected to target (e.g., localize, bridge and/or activate) an immune cell (e.g., an immune effector cell chosen form an NK cell, a T cell, a B cell, a dendritic cell or a macrophage), at a target cell, e.g., a cancer cell, expressing a calreticulin protein (e.g., a wild-type or mutant calreticulin protein), and/or alter the tumor stroma, e.g., alter the tumor microenvironment near the cancer site. Increasing the proximity and/or activity of the immune cell using the multispecific molecules described herein is expected to enhance an immune response against the target cell (e.g., the cancer cell), thereby providing a more effective therapy (e.g., a more effective cancer therapy). Without being bound by theory, a targeted, localized immune response against the target cell (e.g., the cancer cell) is believed to reduce the effects of systemic toxicity of the multispecific molecules described herein.

Accordingly, provided herein are, inter alia, multispecific molecules (e.g., multispecific or multifunctional antibody molecules) that include the aforesaid moieties, nucleic acids encoding the same, methods of producing the aforesaid molecules, and methods of treating a cancer using the aforesaid molecules.

In an aspect, the disclosure features a method of detecting calreticulin (e.g., wild-type or mutant calreticulin) in a sample or subject, comprising: contacting the sample or subject with an anti-calreticulin antibody molecule described herein; and detecting formation of a complex between the antibody molecule and the sample or subject, thereby detecting calreticulin (e.g., wild-type or mutant calreticulin).

In some embodiments, calreticulin (e.g., wild-type or mutant calreticulin) is detected in vitro or in vivo.

In some embodiments, the method further comprises contacting a reference sample or subject with the antibody molecule; and detecting formation of a complex between the antibody molecule and the reference sample or subject, wherein a change, e.g., a statistically significant change, in the formation of the complex in the sample or subject, relative to the reference sample or subject is indicative of the presence of calreticulin (e.g., wild-type or mutant calreticulin) in the sample or subject.

In some embodiments, the method further comprises obtaining a sample from a subject.

In some embodiments, the sample comprises one or more of plasma, tissue (e.g., cancerous tissue), biopsy, blood (e.g., whole blood), PBMCs, bone marrow, and/or lymphatic tissue, e.g., lymph node. In some embodiments, the sample has not been frozen and/or fixed. In some embodiments, the sample has been formalin-fixed (e.g., formalin-fixed, paraffin-embedded (FFPE). In some embodiments, the sample has been stained (e.g., for analysis by immunohistochemistry). In some embodiments, the sample has been frozen and/or fixed.

In some embodiments, the subject has, or is at risk of having, a disease or disorder described herein (e.g., cancer, e.g., a myelofibrosis).

In some embodiments, the method further comprises performing a flow cytometry analysis, e.g., using a multi-panel method. In some embodiments, the method further comprises performing an immunohistochemical (IHC) analysis, e.g. monochrome or in a multiplexed format. In some embodiments, the IHC method comprises brightfield chromogenic IHC. In embodiments, the brightfield chromogenic IHC method comprises direct detection of antigens by primary antibodies, e.g., which are directly labeled with different chromogens. In some embodiments, the IHC method comprises fluorescent IHC. In embodiments, the fluorescent IHC method comprises direct detection of antigens by primary antibodies, e.g., which are directly labeled with different fluorophores. In some embodiments, the method further comprises performing immunohistochemistry on a sample, e.g., a fixed sample, e.g., an FFPE sample. In some embodiments, the method further comprises measuring the level of calreticulin+(e.g., wild-type calreticulin+ or mutant calreticulin+) cells from the biological sample (e.g., determining if calreticulin+(e.g., wild-type calreticulin+ or mutant calreticulin+) cells are depleted, e.g., relative to a reference sample or subject. In some embodiments, the method further comprises measuring the intracellular level of calreticulin (e.g., wild-type or mutant calreticulin). In some embodiments, the method further comprises measuring the membrane level of calreticulin (e.g., wild-type or mutant calreticulin).

In some embodiments, the method comprises combining two or more of the detection methods described herein. In embodiments, the method comprises a nucleic acid-based method and an antibody-based method.

In some embodiments, the method further comprises evaluating the subject for a change in prognosis, severity, or presence or absence of a disease or disorder (e.g., cancer, e.g., myelofibrosis), e.g., after treatment (e.g., with an antibody molecule described herein).

In some embodiments, the antibody molecule is detectably labeled. In some embodiments, the antibody molecule is an anti-calreticulin (e.g., wild-type or mutant calreticulin) antibody molecule.

In an aspect, the disclosure features a method of evaluating a subject, comprising: contacting a sample (e.g., a sample described herein) from the subject with an anti-calreticulin (e.g., wild-type or mutant calreticulin) antibody molecule described herein; and
detecting formation of a complex between the antibody molecule and the sample, thereby evaluating the subject.

In some embodiments, the subject has, or is at risk of having, a disease or disorder described herein (e.g., cancer, e.g., myelofibrosis). In some embodiments, the subject has not been treated with an antibody molecule described herein. In some embodiments, the subject has been treated with an antibody molecule described herein.

In an aspect, the disclosure features a kit comprising an anti-calreticulin (e.g., wild-type or mutant calreticulin) antibody molecule described herein and instructions for use in a method of detecting calreticulin (e.g., wild-type or mutant calreticulin) in a sample or subject, e.g., in accordance with a method described herein.

Accordingly, in one aspect, the disclosure features a multifunctional molecule that includes:
(i) a first antigen binding domain that binds to a calreticulin protein (e.g., a wild-type or mutant calreticulin protein), e.g., a calreticulin-targeting antigen binding domain disclosed in any one of Table 4, Table 5, Table 6, Table 24, Table 25, Table 16, Table 17, Table 18, or Table 19,
and
(ii) a second antigen binding domain that binds to TCRβV, e.g., an anti-TCRβV antigen binding domain disclosed in any one of Table 30, Table 31, Table 32, Table 33, Table 11, Table 12, or Table 13, or a second antigen binding domain that binds to NKp30, e.g., an anti-NKp30 antigen binding domain disclosed in Tables 7, Table 8, Table 35, Table 36, Table 9, Table 10, or Table 34.

In some embodiments, the second antigen binding domain binds to TCRβV.

In some embodiments, the second antigen binding domain activates a T cell or the second antigen binding domain does not activate a T cell.

In some embodiments, the second antigen binding domain binds to TCRβ V12 or TCRβ V6 (e.g., comprising the amino acid sequence of SEQ ID NO: 1044).

In some embodiments, the second antigen binding domain comprises one or more amino acid sequences as listed in Table 30, Table 31, Table 32, Table 33, Table 11, Table 12, or Table 13.

In some embodiments, the second antigen binding domain comprises:
(a) a heavy chain variable region (VH) and/or a light chain variable region (VL), wherein:
(i) the VH comprises a heavy chain complementarity determining region 1 (VHCDR1) having an amino acid sequence of a VHCDR1 in Table 30, Table 31, Table 33, Table 11, Table 12, or Table 13 (or a sequence with no more than 1, 2, 3, or 4 mutations, e.g., substitutions, additions, or deletions), a VHCDR2 having an amino acid sequence of a VHCDR2 in Table 30, Table 31, Table 33, Table 11, Table 12, or Table 13 (or a sequence with no more than 1, 2, 3, or 4 mutations, e.g., substitutions, additions, or deletions), and/or a VHCDR3 having an amino acid sequence of a VHCDR3 in Table 30, Table 31, Table 33, Table 11, Table 12, or Table 13 (or a sequence with no more than 1, 2, 3, or 4 mutations, e.g., substitutions, additions, or deletions),
(ii) the VL comprises a light chain complementarity determining region 1 (VLCDR1) having an amino acid sequence of a VLCDR1 in Table 30, Table 31, Table 33, Table 11, Table 12, or Table 13 (or a sequence with no more than 1, 2, 3, or 4 mutations, e.g., substitutions, additions, or deletions), a VLCDR2 having an amino acid sequence of a VLCDR2 in Table 30, Table 31, Table 33, Table 11, Table 12, or Table 13 (or a sequence with no more than 1, 2, 3, or 4 mutations, e.g., substitutions, additions, or deletions), and/or a VLCDR3 having an amino acid sequence of a VLCDR3 in Table 30, Table 31, Table 33, Table 11, Table 12, or Table 13 (or a sequence with no more than 1, 2, 3, or 4 mutations, e.g., substitutions, additions, or deletions);
(b) a heavy chain variable region (VH) and/or a light chain variable region (VL), wherein:
(i) the VH comprises a heavy chain complementarity determining region 1 (VHCDR1) amino acid sequence of SEQ ID NO: 3 (or a sequence with no more than 1, 2, 3, or 4 mutations, e.g., substitutions, additions, or deletions), a VHCDR2 amino acid sequence of SEQ ID NO: 4 (or a sequence with no more than 1, 2, 3, or 4 mutations, e.g., substitutions, additions, or deletions), and/or a VHCDR3 amino acid sequence of SEQ ID NO: 5 (or a sequence with no more than 1, 2, 3, or 4 mutations, e.g., substitutions, additions, or deletions), and/or
(ii) the VL comprises a light chain complementarity determining region 1 (VHCDR1) amino acid sequence of SEQ ID NO: 6 (or a sequence with no more than 1, 2, 3, or 4 mutations, e.g., substitutions, additions, or deletions), a VHCDR2 amino acid sequence of SEQ ID NO: 7 (or a sequence with no more than 1, 2, 3, or 4 mutations, e.g., substitutions, additions, or deletions), and/or a VHCDR3 amino acid sequence of SEQ ID NO: 8 (or a sequence with no more than 1, 2, 3, or 4 mutations, e.g., substitutions, additions, or deletions);
(c) a heavy chain variable region (VH) and/or a light chain variable region (VL), wherein:
(i) the VH comprises a heavy chain complementarity determining region 1 (VHCDR1) amino acid sequence of SEQ ID NO: 45 (or a sequence with no more than 1, 2, 3, or 4 mutations, e.g., substitutions, additions, or deletions), a VHCDR2 amino acid sequence of SEQ ID NO: 46 (or a sequence with no more than 1, 2, 3, or 4 mutations, e.g., substitutions, additions, or deletions), and/or a VHCDR3 amino acid sequence of SEQ ID NO: 47 (or a sequence with no more than 1, 2, 3, or 4 mutations, e.g., substitutions, additions, or deletions), and/or
(ii) the VL comprises a light chain complementarity determining region 1 (VHCDR1) amino acid sequence of SEQ ID NO: 51 (or a sequence with no more than 1, 2, 3, or 4 mutations, e.g., substitutions, additions, or deletions), a VHCDR2 amino acid sequence of SEQ ID NO: 52 (or a sequence with no more than 1, 2, 3, or 4 mutations, e.g., substitutions, additions, or deletions), and/or a VHCDR3 amino acid sequence of SEQ ID NO: 53 (or a sequence with no more than 1, 2, 3, or 4 mutations, e.g., substitutions, additions, or deletions); and/or
(d) a heavy chain variable region (VH) and/or a light chain variable region (VL), wherein:
(i) the VH comprises a heavy chain complementarity determining region 1 (VHCDR1) amino acid sequence of SEQ ID NO: 48 (or a sequence with no more than 1, 2, 3, or 4 mutations, e.g., substitutions, additions, or deletions), a VHCDR2 amino acid sequence of SEQ ID NO: 49 (or a sequence with no more than 1, 2, 3, or 4 mutations, e.g., substitutions, additions, or deletions), and/or a VHCDR3 amino acid sequence of SEQ ID NO: 50 (or a sequence with no more than 1, 2, 3, or 4 mutations, e.g., substitutions, additions, or deletions), and/or
(ii) the VL comprises a light chain complementarity determining region 1 (VHCDR1) amino acid sequence of SEQ ID NO: 54 (or a sequence with no more than 1, 2, 3, or 4 mutations, e.g., substitutions, additions, or deletions), a VHCDR2 amino acid sequence of SEQ ID NO: 55 (or a sequence with no more than 1, 2, 3, or 4 mutations, e.g., substitutions, additions, or deletions), and/or a VHCDR3 amino acid sequence of SEQ ID NO: 56 (or a sequence with no more than 1, 2, 3, or 4 mutations, e.g., substitutions, additions, or deletions).

In some embodiments, the second antigen binding domain comprises:
(a) a heavy chain variable region (VH) and/or a light chain variable region (VL), wherein:
(i) the VH comprises the amino acid sequence of a VH in Table 30, Table 31, Table 33, Table 11, Table 12, or Table 13 (or an amino acid sequence having at least about 77%, 80%, 85%, 90%, 95%, or 99% sequence identity thereto), and/or
(ii) the VL comprises the amino acid sequence of a VL in Table 30, Table 31, Table 33, Table 11, Table 12, or Table 13 (or an amino acid sequence having at least about 77%, 80%, 85%, 90%, 95%, or 99% sequence identity thereto)
(iii) the VH comprises the amino acid sequence of SEQ ID NO: 9 (or an amino acid sequence having at least about 77%, 80%, 85%, 90%, 95%, or 99% sequence identity thereto), and/or
(iv) the VL comprises the amino acid sequence of SEQ ID NO: 10 (or an amino acid sequence having at least about 77%, 80%, 85%, 90%, 95%, or 99% sequence identity thereto);
(b) a heavy chain variable region (VH) and/or a light chain variable region (VL), wherein:
(i) the VH comprises the amino acid sequence of SEQ ID NO: 9 (or an amino acid sequence having at least about 77%, 80%, 85%, 90%, 95%, or 99% sequence identity thereto), and/or
(ii) the VL comprises the amino acid sequence of SEQ ID NO: 11 (or an amino acid sequence having at least about 77%, 80%, 85%, 90%, 95%, or 99% sequence identity thereto); and/or
(c) a heavy chain variable region (VH) and/or a light chain variable region (VL), wherein:
(i) the VH comprises the amino acid sequence of SEQ ID NO: 1312 (or an amino acid sequence having at least about 77%, 80%, 85%, 90%, 95%, or 99% sequence identity thereto), and/or
(ii) the VL comprises the amino acid sequence of SEQ ID NO: 1314 (or an amino acid sequence having at least about 77%, 80%, 85%, 90%, 95%, or 99% sequence identity thereto).

In some embodiments, the second antigen binding domain comprises:
(a) a heavy chain variable region (VH) and/or a light chain variable region (VL), wherein:
(i) the VH comprises a heavy chain complementarity determining region 1 (VHCDR1) amino acid sequence of SEQ ID NO: 17 (or a sequence with no more than 1, 2, 3, or 4 mutations, e.g., substitutions, additions, or deletions), a VHCDR2 amino acid sequence of SEQ ID NO: 18 (or a sequence with no more than 1, 2, 3, or 4 mutations, e.g., substitutions, additions, or deletions), and/or a VHCDR3 amino acid sequence of SEQ ID NO: 19 (or a sequence with no more than 1, 2, 3, or 4 mutations, e.g., substitutions, additions, or deletions), and/or
(ii) the VL comprises a light chain complementarity determining region 1 (VHCDR1) amino acid sequence of SEQ ID NO: 20 (or a sequence with no more than 1, 2, 3, or 4 mutations, e.g., substitutions, additions, or deletions), a VHCDR2 amino acid sequence of SEQ ID NO: 21 (or a sequence with no more than 1, 2, 3, or 4 mutations, e.g., substitutions, additions, or deletions), and/or a VHCDR3 amino acid sequence of SEQ ID NO: 22 (or a sequence with no more than 1, 2, 3, or 4 mutations, e.g., substitutions, additions, or deletions);
(b) a heavy chain variable region (VH) and/or a light chain variable region (VL), wherein:
(i) the VH comprises a heavy chain complementarity determining region 1 (VHCDR1) amino acid sequence of SEQ ID NO: 57 (or a sequence with no more than 1, 2, 3, or 4 mutations, e.g., substitutions, additions, or deletions), a VHCDR2 amino acid sequence of SEQ ID NO: 58 (or a sequence with no more than 1, 2, 3, or 4 mutations, e.g., substitutions, additions, or deletions), and/or a VHCDR3 amino acid sequence of SEQ ID NO: 59 (or a sequence with no more than 1, 2, 3, or 4 mutations, e.g., substitutions, additions, or deletions), and/or
(ii) the VL comprises a light chain complementarity determining region 1 (VHCDR1) amino acid sequence of SEQ ID NO: 63 (or a sequence with no more than 1, 2, 3, or 4 mutations, e.g., substitutions, additions, or deletions), a VHCDR2 amino acid sequence of SEQ ID NO: 64 (or a sequence with no more than 1, 2, 3, or 4 mutations, e.g., substitutions, additions, or deletions), and/or a VHCDR3 amino acid sequence of SEQ ID NO: 65 (or a sequence with no more than 1, 2, 3, or 4 mutations, e.g., substitutions, additions, or deletions); and/or
(c) a heavy chain variable region (VH) and/or a light chain variable region (VL), wherein:
(i) the VH comprises a heavy chain complementarity determining region 1 (VHCDR1) amino acid sequence of SEQ ID NO: 60 (or a sequence with no more than 1, 2, 3, or 4 mutations, e.g., substitutions, additions, or deletions), a VHCDR2 amino acid sequence of SEQ ID NO: 61 (or a sequence with no more than 1, 2, 3, or 4 mutations, e.g., substitutions, additions, or deletions), and/or a VHCDR3 amino acid sequence of SEQ ID NO: 62 (or a sequence with no more than 1, 2, 3, or 4 mutations, e.g., substitutions, additions, or deletions), and/or
(ii) the VL comprises a light chain complementarity determining region 1 (VHCDR1) amino acid sequence of SEQ ID NO: 66 (or a sequence with no more than 1, 2, 3, or 4 mutations, e.g., substitutions, additions, or deletions), a VHCDR2 amino acid sequence of SEQ ID NO: 67 (or a sequence with no more than 1, 2, 3, or 4 mutations, e.g., substitutions, additions, or deletions), and/or a VHCDR3 amino acid sequence of SEQ ID NO: 68 (or a sequence with no more than 1, 2, 3, or 4 mutations, e.g., substitutions, additions, or deletions).

In some embodiments, the second antigen binding domain comprises:
(a) a heavy chain variable region (VH) and/or a light chain variable region (VL), wherein:
(i) the VH comprises the amino acid sequence of SEQ ID NO: 15 (or an amino acid sequence having at least about 77%, 80%, 85%, 90%, 95%, or 99% sequence identity thereto), and/or
(ii) the VL comprises the amino acid sequence of SEQ ID NO: 16 (or an amino acid sequence having at least about 77%, 80%, 85%, 90%, 95%, or 99% sequence identity thereto); and/or
(b) a heavy chain variable region (VH) and/or a light chain variable region (VL), wherein:
(i) the VH comprises: the amino acid sequence of SEQ ID NO: 23 (or an amino acid sequence having at least about 77%, 80%, 85%, 90%, 95%, or 99% sequence identity thereto), the amino acid sequence of SEQ ID NO: 24 (or an amino acid sequence having at least about 77%, 80%, 85%, 90%, 95%, or 99% sequence identity thereto), or the amino acid sequence of SEQ ID NO: 25 (or an amino acid sequence having at least about 77%, 80%, 85%, 90%, 95%, or 99% sequence identity thereto); and/or
(ii) the VL comprises: the amino acid sequence of SEQ ID NO: 26 (or an amino acid sequence having at least about 77%, 80%, 85%, 90%, 95%, or 99% sequence identity thereto), the amino acid sequence of SEQ ID NO: 27 (or an amino acid sequence having at least about 77%, 80%, 85%, 90%, 95%, or 99% sequence identity thereto), the amino acid sequence of SEQ ID NO: 28 (or an amino acid sequence having at least about 77%, 80%, 85%, 90%, 95%, or 99% sequence identity thereto), the amino acid sequence of SEQ ID NO: 29 (or an amino acid sequence having at least about 77%, 80%, 85%, 90%, 95%, or 99% sequence identity thereto), or the amino acid sequence of SEQ ID NO: 30 (or an amino acid sequence having at least about 77%, 80%, 85%, 90%, 95%, or 99% sequence identity thereto).

In some embodiments, the multifunctional molecule comprises: a first polypeptide comprising, e.g., from N-terminus to C-terminus, a first VL and a first CL, a second polypeptide comprising, e.g., from N-terminus to C-terminus, a first VH, a first CH1, a first dimerization domain (e.g., a first Fc), and a first moiety that binds to TCR (e.g., TCRVβ) (e.g., a first scFv that binds to TCR (e.g., TCRVβ)),
a third polypeptide comprising, e.g., from N-terminus to C-terminus, a second VH, a second CH1, a second dimerization domain (e.g., a second Fc), and optionally a second moiety that binds to TCR (e.g., TCRVβ) (e.g., a second scFv that binds to TCR (e.g., TCRVβ)),
a fourth polypeptide comprising, e.g., from N-terminus to C-terminus, a second VL and a second CL, wherein:
the first VL and the first VH form a first antigen binding domain that binds to a first calreticulin protein, and the second VL and the second VH form a third antigen binding domain that binds to a second calreticulin protein,
optionally wherein the first and second calreticulin proteins comprise the amino acid sequence of SEQ ID NO: 6285, 1001, or 6286,
optionally wherein the first and second calreticulin mutant proteins are each independently chosen from: a molecule comprising the amino acid sequence of SEQ ID NO: 6313, or a molecule comprising the amino acid sequence of SEQ ID NO: 6314, optionally wherein the multifunctional molecule comprises the configuration of FIG. 3A or 3B.

In some embodiments, the second antigen binding domain binds to NKp30.

In some embodiments, the second antigen binding domain is chosen from an antibody molecule, e.g., an antigen binding domain, or ligand that binds to (e.g., activates) NKp30, e.g., the second antigen binding domain is an antibody molecule or ligand that binds to (e.g., activates) NKp30.

In some embodiments, the second antigen binding domain comprises:
(i) a heavy chain variable region (VH) comprising a heavy chain complementarity determining region 1 (VHCDR1) having an amino acid sequence of a VHCDR1 of Table 7, Table 35, Table 9, Table 10, or Table 34 (or a sequence with no more than 1, 2, 3, or 4 mutations, e.g., substitutions, additions, or deletions), a VHCDR2 having an amino acid sequence of a VHCDR2 of Table 7, Table 35, Table 9, Table 10, or Table 34 (or a sequence with no more than 1, 2, 3, or 4 mutations, e.g., substitutions, additions, or deletions), and/or a VHCDR3 having an amino acid sequence of a VHCDR3 of Table 7, Table 35, Table 9, Table 10, or Table 34 (or a sequence with no more than 1, 2, 3, or 4 mutations, e.g., substitutions, additions, or deletions), and/or
(ii) a light chain variable region (VL) comprising a light chain complementarity determining region 1 (VLCDR1) having an amino acid sequence of a VLCDR1 of Table 8, Table 36, Table 9, Table 10, or Table 34 (or a sequence with no more than 1, 2, 3, or 4 mutations, e.g., substitutions, additions, or deletions), a VLCDR2 having an amino acid sequence of a VLCDR2 of Table 8, Table 36, Table 9, Table 10, or Table 34 (or a sequence with no more than 1, 2, 3, or 4 mutations, e.g., substitutions, additions, or deletions), and/or a VLCDR3 having an amino acid sequence of a VLCDR3 of Table 8, Table 36, Table 9, Table 10, or Table 34 (or a sequence with no more than 1, 2, 3, or 4 mutations, e.g., substitutions, additions, or deletions).

In some embodiments, the second antigen binding domain comprises:
(i) a heavy chain variable region (VH) comprising a heavy chain complementarity determining region 1 (VHCDR1) amino acid sequence of SEQ ID NO: 7313 (or a sequence with no more than 1, 2, 3, or 4 mutations, e.g., substitutions, additions, or deletions), a VHCDR2 amino acid sequence of SEQ ID NO: 6001 (or a sequence with no more than 1, 2, 3, or 4 mutations, e.g., substitutions, additions, or deletions, and/or a VHCDR3 amino acid sequence of SEQ ID NO: 7315 (or a sequence with no more than 1, 2, 3, or 4 mutations, e.g., substitutions, additions, or deletions; and/or
(ii) a light chain variable region (VL) comprising a light chain complementarity determining region 1 (VLCDR1) amino acid sequence of SEQ ID NO: 7326 (or a sequence with no more than 1, 2, 3, or 4 mutations, e.g., substitutions, additions, or deletions), a VLCDR2 amino acid sequence of SEQ ID NO: 7327 (or a sequence with no more than 1, 2, 3, or 4 mutations, e.g., substitutions, additions, or deletions), and/or a VLCDR3 amino acid sequence of SEQ ID NO: 7329 (or a sequence with no more than 1, 2, 3, or 4 mutations, e.g., substitutions, additions, or deletions).

In some embodiments, the second antigen binding domain comprises:
(i) a VH comprising the amino acid sequence of any of SEQ ID NOs: 7298 or 7300-7304 (or an amino acid sequence having at least about 75%, 80%, 85%, 90%, 95%, or 99% sequence identity to any of SEQ ID NOs: 7298 or 7300-7304); and/or
(ii) a VL comprising the amino acid sequence of any of SEQ ID NOs: 7299 or 7305-7309 (or an amino acid sequence having at least about 93%, 95%, or 99% sequence identity to any of SEQ ID NOs: 7299 or 7305-7309).

In some embodiments, the second antigen binding domain comprises:
(i) a VH comprising the amino acid sequence of SEQ ID NO: 7302 (or an amino acid sequence having at least about 75%, 80%, 85%, 90%, 95%, or 99% sequence identity to 7302), and a VL comprising the amino acid sequence of SEQ ID NO: 7305 (or an amino acid sequence having at least about 75%, 80%, 85%, 90%, 95%, or 99% sequence identity to 7305); or
(ii) a VH comprising the amino acid sequence of SEQ ID NO: 7302 (or an amino acid sequence having at least about 75%, 80%, 85%, 90%, 95%, or 99% sequence identity to 7302), and a VL comprising the amino acid sequence of SEQ ID NO: 7309 (or an amino acid sequence having at least about 75%, 80%, 85%, 90%, 95%, or 99% sequence identity to 7309).

In some embodiments, the second antigen binding domain comprises:
(i) an amino acid sequence of SEQ ID NO: 7310 (or an amino acid sequence having at least about 75%, 80%, 85%, 90%, 95%, or 99% sequence identity to 7310); or
(ii) an amino acid sequence of SEQ ID NO: 7311 (or an amino acid sequence having at least about 75%, 80%, 85%, 90%, 95%, or 99% sequence identity to 7311).

In some embodiments, the second antigen binding domain comprises:
(i) a heavy chain variable region (VH) comprising a heavy chain complementarity determining region 1 (VHCDR1) amino acid sequence of SEQ ID NO: 6000, a VHCDR2 amino acid sequence of SEQ ID NO: 6001, and/or a VHCDR3 amino acid sequence of SEQ ID NO: 6002, and
(ii) a light chain variable region (VL) comprising a light chain complementarity determining region 1 (VLCDR1) amino acid sequence of SEQ ID NO: 6063, a VLCDR2 amino acid sequence of SEQ ID NO: 6064, and/or a VLCDR3 amino acid sequence of SEQ ID NO: 7293.

In some embodiments, the second antigen binding domain comprises:
(1) a heavy chain variable region (VH) comprising a heavy chain framework region 1 (VHFWR1) having an amino acid sequence of a VHFWR1 of Table 7, Table 35, Table 9, Table 10, or Table 34 (or a sequence with no more than 1, 2, 3, 4, 5, or 6 mutations, e.g., substitutions, additions, or deletions, therefrom), a VHFWR2 having an amino acid sequence of a VHFWR2 of Table 7, Table 35, Table 9, Table 10, or Table 34 (or a sequence with no more than 1, 2, 3, 4, 5, or 6 mutations, e.g., substitutions, additions, or deletions, therefrom), a VHFWR3 having an amino acid sequence of a VHFWR3 of Table 7, Table 35, Table 9, Table 10, or Table 34 (or a sequence with no more than 1, 2, 3, 4, 5, or 6 mutations, e.g., substitutions, additions, or deletions, therefrom), or a VHFWR4 having an amino acid sequence of a VHFWR4 of Table 7, Table 35, Table 9, Table 10, or Table 34 (or a sequence with no more than 1, 2, 3, 4, 5, or 6 mutations, e.g., substitutions, additions, or deletions, therefrom), and/or
(2) a light chain variable region (VL) comprising a light chain framework region 1 (VLFWR1) having an amino acid sequence of a VLFWR1 of Table 8, Table 36, Table 9, Table 10, or Table 34 (or a sequence with no more than 1, 2, 3, 4, 5, or 6 mutations, e.g., substitutions, additions, or deletions, therefrom), a VLFWR2 having an amino acid sequence of a VLFWR2 of Table 8, Table 36, Table 9, Table 10, or Table 34 (or a sequence with no more than 1, 2, 3, 4, 5, or 6 mutations, e.g., substitutions, additions, or deletions, therefrom), a VLFWR3 having an amino acid sequence of a VLFWR3 of Table 8, Table 36, Table 9, Table 10, or Table 34 (or a sequence with no more than 1, 2, 3, 4, 5, or 6 mutations, e.g., substitutions, additions, or deletions, therefrom), or a VLFWR4 having an amino acid sequence of a VLFWR4 of Table 8, Table 36, Table 9, Table 10, or Table 34 (or a sequence with no more than 1, 2, 3, 4, 5, or 6 mutations, e.g., substitutions, additions, or deletions, therefrom).

In some embodiments, the second antigen binding domain comprises:
(1) a heavy chain variable region (VH) comprising a heavy chain framework region 1 (VHFWR1) amino acid sequence of SEQ ID NO: 6003, a VHFWR2 amino acid sequence of SEQ ID NO: 6004, a VHFWR3 amino acid sequence of SEQ ID NO: 6005, or a VHFWR4 amino acid sequence of SEQ ID NO: 6006, and
(3) a light chain variable region (VL) comprising a light chain framework region 1 (VLFWR1) amino acid sequence of SEQ ID NO: 6066, a VLFWR2 amino acid sequence of SEQ ID NO: 6067, a VLFWR3 amino acid sequence of SEQ ID NO: 7292, or a VLFWR4 amino acid sequence of SEQ ID NO: 6069.

In some embodiments, the second antigen binding domain comprises:
(i) a VH comprising the amino acid sequence of a VH of Table 7, Table 35, Table 9, Table 10, or Table 34 (or an amino acid sequence having at least about 75%, 80%, 85%, 90%, 95%, or 99% sequence identity thereto), and/or
(ii) a VL comprising the amino acid sequence of a VL of Table 8, Table 36, Table 9, Table 10, or Table 34 (or an amino acid sequence having at least about 93%, 95%, or 99% sequence identity thereto).

In some embodiments, the second antigen binding domain comprises a heavy chain comprising the amino acid sequence of a heavy chain of Table 10 (or an amino acid sequence having at least about 75%, 80%, 85%, 90%, 95%, or 99% sequence identity thereto).

In some embodiments, the second antigen binding domain comprises a light chain comprising the amino acid sequence of a light chain of Table 10 (or an amino acid sequence having at least about 75%, 80%, 85%, 90%, 95%, or 99% sequence identity thereto).

In some embodiments, the second antigen binding domain comprises a heavy chain comprising the amino acid sequence of a heavy chain of Table 10 (or an amino acid sequence having at least about 75%, 80%, 85%, 90%, 95%, or 99% sequence identity thereto), and a light chain comprising the amino acid sequence of a light chain of Table 10 (or an amino acid sequence having at least about 75%, 80%, 85%, 90%, 95%, or 99% sequence identity thereto).

In some embodiments, the multispecific molecule comprises:
a first polypeptide comprising, e.g., from N-terminus to C-terminus, a first VL and a first CL, a second polypeptide comprising, e.g., from N-terminus to C-terminus, a first VH, a first CH1, a first dimerization domain (e.g., a first Fc), and a first moiety that binds to NKp30 (e.g., a first antibody molecule or ligand that binds to NKp30),
a third polypeptide comprising, e.g., from N-terminus to C-terminus, a second VH, a second CH1, a second dimerization domain (e.g., a second Fc), and optionally a second moiety that binds to NKp30 (e.g., a second antibody molecule or ligand that binds to NKp30), a fourth polypeptide comprising, e.g., from N-terminus to C-terminus, a second VL and a second CL, wherein:
the first VL and the first VH form a first antigen binding domain that binds to a first calreticulin protein, and the second VL and the second VH from a third antigen binding domain that binds to a second calreticulin protein,
optionally wherein the first and second calreticulin proteins comprise the amino acid sequence of SEQ ID NO: 6285, 1001, or 6286,
optionally wherein the first and second calreticulin mutant proteins are each independently chosen from: a molecule comprising the amino acid sequence of SEQ ID NO: 6313, or a molecule comprising the amino acid sequence of SEQ ID NO: 6314, optionally wherein the multifunctional molecule comprises the configuration of FIG. 3A or 3B.

In some embodiments, the calreticulin protein comprises an amino acid sequence chosen from SEQ ID NOs: 6285-6312 or 1001, optionally wherein the calreticulin protein comprises an amino acid sequence chosen from SEQ ID NOs: 6313-6346 or 1002-1003.

In some embodiments, the calreticulin protein comprises the amino acid sequence of SEQ ID NO: 6285 or 1001.

In some embodiments, the calreticulin protein comprises the amino acid sequence of SEQ ID NO: 6286.

In some embodiments, the first antigen binding domain binds to an epitope located within the C-terminus of the calreticulin protein, optionally wherein the first antigen binding domain binds to an epitope located within the amino acid sequence of SEQ ID NO: 6285, 1001, or 6286.

In some embodiments, the multispecific molecule further comprises: a third antigen binding domain that binds to a second calreticulin protein, e.g., wherein the second calreticulin mutant protein comprises the amino acid sequence of SEQ ID NO: 6285, 1001, or 6286, optionally wherein:
(i) the third antigen binding domain is different from the first antigen binding domain, or
(ii) the third antigen binding domain is the same as the first antigen binding domain.

In some embodiments, the second calreticulin molecule is the same as the calreticulin molecule bound by the first antigen binding domain.

In some embodiments, the second calreticulin molecule is different from the calreticulin molecule bound by the first antigen binding domain.

In some embodiments, the second calreticulin protein comprises an amino acid sequence chosen from SEQ ID NOs: 6285-6312 or 1001, optionally wherein the second calreticulin protein comprises an amino acid sequence chosen from SEQ ID NOs: 6313-6346 or 1002-1003.

In some embodiments, the calreticulin protein bound by the first antigen binding domain comprises the amino acid sequence of SEQ ID NO: 6285 or 1001, and the second calreticulin protein comprises the amino acid sequence of SEQ ID NO: 6286.

In some embodiments, the third antigen binding domain binds to an epitope located within the C-terminus of the second calreticulin protein, optionally wherein the third antigen binding domain binds to an epitope located within the amino acid sequence of SEQ ID NO: 6285, 1001, or 6286.

In some embodiments, the first antigen binding domain comprises:
(i) a heavy chain variable region (VH) comprising a heavy chain complementarity determining region 1 (VHCDR1) having an amino acid sequence of a VHCDR1 in Table 4, Table 24, Table 25, or Table 17 (or a sequence with no more than 1, 2, 3, or 4 mutations, e.g., substitutions, additions, or deletions), a VHCDR2 having an amino acid sequence of a VHCDR2 in Table 4, Table 24, Table 25, or Table 17 (or a sequence with no more than 1, 2, 3, or 4 mutations, e.g., substitutions, additions, or deletions), and/or a VHCDR3 having an amino acid sequence of a VHCDR3 in Table 4, Table 24, Table 25, or Table 17 (or a sequence with no more than 1, 2, 3, or 4 mutations, e.g., substitutions, additions, or deletions);
(ii) a light chain variable region (VL) comprising a light chain complementarity determining region 1 (VHCDR1) having an amino acid sequence of a VLCDR1 in Table 5, Table 24, Table 25, or Table 18 (or a sequence with no more than 1, 2, 3, or 4 mutations, e.g., substitutions, additions, or deletions), a VHCDR2 having an amino acid sequence of a VLCDR2 in Table 5, Table 24, Table 25, or Table 18 (or a sequence with no more than 1, 2, 3, or 4 mutations, e.g., substitutions, additions, or deletions), and/or a VHCDR3 having an amino acid sequence of a VLCDR3 in Table 5, Table 24, Table 25, or Table 18 (or a sequence with no more than 1, 2, 3, or 4 mutations, e.g., substitutions, additions, or deletions);
(iii) a VH comprising the amino acid sequence of a VH in Table 24, Table 25, or Table 16 (or an amino acid sequence having at least about 77%, 80%, 85%, 90%, 95%, or 99% sequence identity thereto);
(iv) a VL comprising the amino acid sequence of a VL in Table 24, Table 25, or Table 16 (or an amino acid sequence having at least about 93%, 95%, or 99% sequence identity thereto);
(v) a VH comprising a heavy chain framework region 1 (VHFWR1) having an amino acid sequence of a VHFWR1 in Table 4 or Table 6 (or a sequence with no more than 1, 2, 3, 4, 5, 6, 7, 8, or 9 mutations, e.g., substitutions, additions, or deletions), a VHFWR2 having an amino acid sequence of a VHFWR2 in Table 4 or Table 6 (or a sequence with no more than 1, 2, 3, 4, 5, 6, 7, 8, or 9 mutations, e.g., substitutions, additions, or deletions), a VHFWR3 having an amino acid sequence of a VHFWR3 in Table 4 or Table 6 (or a sequence with no more than 1, 2, 3, 4, 5, 6, 7, 8, or 9 mutations, e.g., substitutions, additions, or deletions), and/or a VHFWR4 having an amino acid sequence of a VHFWR4 in Table 4 or Table 6 (or a sequence with no more than 1, 2, 3, 4, 5, 6, 7, 8, or 9 mutations, e.g., substitutions, additions, or deletions), and/or
(vi) a VL comprising a light chain framework region 1 (VLFWR1) having an amino acid sequence of a VLFWR1 in Table 5 or Table 6 (or a sequence with no more than 1, 2, 3, 4, 5, 6, 7, 8, or 9 mutations, e.g., substitutions, additions, or deletions), a VLFWR2 having an amino acid sequence of a VLFWR2 in Table 5 or Table 6 (or a sequence with no more than 1, 2, 3, 4, 5, 6, 7, 8, or 9 mutations, e.g., substitutions, additions, or deletions), a VLFWR3 having an amino acid sequence of a VLFWR3 in Table 5 or Table 6 (or a sequence with no more than 1, 2, 3, 4, 5, 6, 7, 8, or 9 mutations, e.g., substitutions, additions, or deletions), and/or a VLFWR4 having an amino acid sequence of a VLFWR4 in Table 5 or Table 6 (or a sequence with no more than 1, 2, 3, 4, 5, 6, 7, 8, or 9 mutations, e.g., substitutions, additions, or deletions).

In some embodiments, the multifunctional molecule further comprises a tumor-targeting moiety.

In some embodiments, the tumor-targeting moiety binds to a tumor antigen.

In some embodiments, the tumor antigen is selected from G6B, CD34, CD41, P-selectin, Clec2, cKIT, FLT3, MPL, ITGB3, ITGB2, GP5, GP6, GP9, GP1BA, DSC2, FCGR2A, TNFRSF10A, TNFRSF10B, or TM4SF1.

In some embodiments, the tumor-targeting moiety comprises an antibody molecule, e.g., that binds to a tumor antigen selected from G6B, CD34, CD41, P-selectin, Clec2, cKIT, FLT3, MPL, ITGB3, ITGB2, GP5, GP6, GP9, GP1BA, DSC2, FCGR2A, TNFRSF10A, TNFRSF10B, or TM4SF1.

In some embodiments, the tumor-targeting moiety comprises a VH and/or VL sequence, e.g., as listed in Table 38 or Table 20.

In some embodiments, the multifunctional molecule preferentially binds to a myeloproliferative neoplasm cell over a non-tumor cell, optionally wherein the binding between the multifunctional molecule and the myeloproliferative neoplasm cell is more than 10, 20, 30, 40, 50-fold greater than the binding between the multifunctional molecule and a non-tumor cell.

In some embodiments, the myeloproliferative neoplasm cell is chosen from a myelofibrosis cell, an essential thrombocythemia cell, a polycythemia vera cell, or a chronic myeloid cancer cell, optionally wherein: the myeloproliferative neoplasm cell does not comprise a JAK2 V617F mutation, or the myeloproliferative neoplasm cell does not comprise a MPL mutation.

In some embodiments, the multispecific molecule further comprises a linker, e.g., a linker between the first antigen binding domain and the second antigen binding domain.

In some embodiments, the linker is chosen from: a cleavable linker, a non-cleavable linker, a peptide linker, a flexible linker, a rigid linker, a helical linker, or a non-helical linker.

In some embodiments, the linker is a peptide linker.

In some embodiments, the peptide linker comprises Gly and Ser.

In some embodiments, the peptide linker comprises an amino acid sequence chosen from SEQ ID NOs: 6214-6217 or 6220-6221 and 77-78.

In another aspect, the disclosure provides a nucleic acid molecule encoding the multifunctional molecule as described herein.

In another aspect, the disclosure provides a vector, e.g., an expression vector, comprising the nucleic acid molecule as described herein.

In another aspect, the disclosure provides a host cell comprising the nucleic acid molecule or a vector as described herein.

In another aspect, the disclosure provides a method of making, e.g., producing, the multifunctional molecule as described herein, comprising culturing the host cell described herein, under suitable conditions, e.g., conditions suitable for gene expression and/or homo- or heterodimerization.

In another aspect, the disclosure provides a pharmaceutical composition comprising the multifunctional molecule as described herein and a pharmaceutically acceptable carrier, excipient, or stabilizer.

In another aspect, the disclosure provides a method of treating a cancer, comprising administering to a subject in need thereof the multifunctional molecule as disclosed herein, wherein the multifunctional molecule is administered in an amount effective to treat the cancer.

In another aspect, the disclosure provides a use of the multifunctional molecule as described herein in treating a cancer. In another aspect, the disclosure provides a multifunctional molecule disclosed herein for use in treating a cancer.

In some embodiments, the subject has cancer cells that express the first and/or second calreticulin protein.

In some embodiments, wherein the subject has the JAK2 V617F mutation.

In some embodiments, the subject does not have the JAK2 V617F mutation.

In some embodiments, the subject has an MPL mutation.

In some embodiments, the subject does not have an MPL mutation.

In some embodiments, the cancer is a hematological cancer, optionally wherein the cancer is a myeloproliferative neoplasm, e.g., primary or idiopathic myelofibrosis (MF), essential thrombocytosis (ET), polycythemia vera (PV), or chronic myelogenous leukemia (CML), optionally wherein the cancer is myelofibrosis.

In some embodiments, the cancer is a solid tumor cancer.

In some embodiments, the method or use further comprises administering a second therapeutic treatment.

In some embodiments, the second therapeutic treatment comprises a therapeutic agent (e.g., a chemotherapeutic agent, a biologic agent, hormonal therapy), radiation, or surgery.

In some embodiments, the therapeutic agent is selected from: a chemotherapeutic agent, or a biologic agent.

In another aspect, the disclosure features a multifunctional molecule (e.g., polypeptide or nucleic acid encoding the same) that includes:
(i) a first antigen binding domain that binds to a calreticulin protein (e.g., a wild-type or mutant calreticulin protein), and
(ii) one, two, or all of:
(a) an immune cell engager chosen from a T cell engager, an NK cell engager, a B cell engager, a dendritic cell engager, or a macrophage cell engager;
(b) a cytokine molecule;
(c) a stromal modifying moiety; or
(d) a tumor-targeting moiety that binds to a tumor antigen, e.g., chosen from: G6B, CD34, CD41, P-selectin, Clec2, cKIT, FLT3, MPL, ITGB3, ITGB2, GP5, GP6, GP9, GP1BA, DSC2, FCGR2A, TNFRSF10A, TNFRSF10B, or TM4SF1.

In an aspect, the disclosure features a multifunctional molecule (e.g., polypeptide or nucleic acid encoding the same) that includes:
(i) a first antigen binding domain that binds to a calreticulin protein (e.g., a wild-type or mutant calreticulin protein), and
(ii) a second antigen binding domain comprising an immune cell engager (e.g., a T cell engager, e.g., an antigen binding domain that binds to TCRβV, e.g., as described herein).

In an aspect, the disclosure features a multifunctional molecule (e.g., polypeptide or nucleic acid encoding the same) that includes:
(i) a first antigen binding domain that binds to a calreticulin protein (e.g., a wild-type or mutant calreticulin protein), and
(ii) a second antigen binding domain comprising a tumor-targeting moiety, e.g., that binds to a tumor antigen chosen from: G6B, CD34, CD41, P-selectin, Clec2, cKIT, FLT3, MPL, ITGB3, ITGB2, GP5, GP6, GP9, GP1BA, DSC2, FCGR2A, TNFRSF10A, TNFRSF10B, or TM4SF1.

In an aspect, the disclosure features a multifunctional molecule (e.g., polypeptide or nucleic acid encoding the same) that includes:
(i) a first antigen binding domain that binds to a calreticulin protein (e.g., a wild-type or mutant calreticulin protein),
(ii) a second antigen binding domain comprising an immune cell engager (e.g., a T cell engager, e.g., an antigen binding domain that binds to TCRβV, e.g., as described herein, e.g., an anti-TCRβV antibody molecule described herein), and
(iii) a third antigen binding domain comprising a tumor-targeting moiety, e.g., that binds to a tumor antigen chosen from: G6B, CD34, CD41, P-selectin, Clec2, cKIT, FLT3, MPL, ITGB3, ITGB2, GP5, GP6, GP9, GP1BA, DSC2, FCGR2A, TNFRSF10A, TNFRSF10B, or TM4SF1.

In some embodiments, the multifunctional molecule further comprises a cytokine molecule or a modulator of a cytokine molecule, e.g., a TGF-β inhibitor, e.g., as described herein.

In some embodiments, the multifunctional molecule further comprises an NK cell engager, e.g., an antigen binding domain that binds to Nkp30, e.g., as described herein.

In some embodiments, the calreticulin protein (e.g., the wild-type or mutant calreticulin protein) comprises the amino acid sequence of SEQ ID NO: 6285, 1001, or 6286. In some embodiments, the wild type calreticulin protein comprises the amino acid sequence of SEQ ID NO: 6285 or 1001. In some embodiments, the calreticulin mutant protein comprises the amino acid sequence of SEQ ID NO: 6286.

In some embodiments, the first antigen binding domain comprises a heavy chain variable region (VH) comprising a heavy chain framework region 1 (VHFWR1) amino acid sequence of SEQ ID NO: 6224, a VHFWR2 amino acid sequence of SEQ ID NO: 6226, a VHFWR3 amino acid sequence of SEQ ID NO: 6228, or a VHFWR4 amino acid sequence of SEQ ID NO: 6230. In some embodiments, the first antigen binding domain comprises a heavy chain variable region (VH) comprising a heavy chain framework region 1 (VHFWR1) amino acid sequence of SEQ ID NO: 6232, a VHFWR2 amino acid sequence of SEQ ID NO: 6234, a VHFWR3 amino acid sequence of SEQ ID NO: 6236, or a VHFWR4 amino acid sequence of SEQ ID NO: 6230. In some embodiments, the first antigen binding domain comprises a light chain variable region (VL) comprising a light chain framework region 1 (VLFWR1) amino acid sequence of SEQ ID NO: 6238, a VLFWR2 amino acid sequence of SEQ ID NO: 6240, a VLFWR3 amino acid sequence of SEQ ID NO: 6242, or a VLFWR4 amino acid sequence of SEQ ID NO: 6244.

In some embodiments, the calreticulin protein (e.g., a wild-type or mutant calreticulin protein) comprises an amino acid sequence chosen from SEQ ID NOs: 6285-6312 or 1001. In some embodiments, the calreticulin protein (e.g., a wild-type or mutant calreticulin protein) comprises an amino acid sequence chosen from SEQ ID NOs: 6313-6346 or 1002-1003. In some embodiments, the calreticulin protein (e.g., a wild-type or mutant calreticulin protein) is a calreticulin protein (e.g., a wild-type or mutant calreticulin protein) disclosed in Table 2 or 3. In some embodiments, the calreticulin protein (e.g., a wild-type or mutant calreticulin protein) comprises the amino acid sequence of SEQ ID NO: 6287. In some embodiments, the calreticulin protein (e.g., a wild-type or mutant calreticulin protein) comprises the amino acid sequence of SEQ ID NO: 6313. In some embodiments, the calreticulin protein (e.g., a wild-type or mutant calreticulin protein) comprises the amino acid sequence of SEQ ID NO: 6288. In some embodiments, the calreticulin protein (e.g., a wild-type or mutant calreticulin protein) comprises the amino acid sequence of SEQ ID NO: 6314.

In some embodiments, the multifunctional molecule further comprising a second antigen binding domain that preferentially binds to a second calreticulin protein (e.g., a wild-type or mutant calreticulin protein). In some embodiments, the second calreticulin protein (e.g., a wild-type or mutant calreticulin protein) comprises the amino acid sequence of SEQ ID NO: 6286. In some embodiments, the second antigen binding domain is different from the first antigen binding domain. In some embodiments, the second antigen binding domain is the same as the first antigen binding domain. In some embodiments, the second calreticulin protein (e.g., a wild-type or mutant calreticulin protein) comprises an amino acid sequence chosen from SEQ ID NOs: 6287-6312. In some embodiments, the second calreticulin protein (e.g., a wild-type or mutant calreticulin protein) comprises an amino acid sequence chosen from SEQ ID NOs: 6313-6346 or 1002-1003. In some embodiments, the second calreticulin protein (e.g., a wild-type or mutant calreticulin protein) is a calreticulin protein (e.g., a wild-type or mutant calreticulin protein) disclosed in Table 2 or 3. In some embodiments, the second calreticulin protein comprises the amino acid sequence of SEQ ID NO: 6287. In some embodiments, the second calreticulin protein comprises the amino acid sequence of SEQ ID NO: 6313. In some embodiments, the second calreticulin protein comprises the amino acid sequence of SEQ ID NO: 6288. In some embodiments, the second calreticulin protein comprises the amino acid sequence of SEQ ID NO: 6314.

In some embodiments, the first calreticulin protein (e.g., a wild-type or mutant calreticulin protein) is a Type 1 calreticulin protein (e.g., a wild-type or mutant calreticulin protein), and the second calreticulin protein (e.g., a wild-type or mutant calreticulin protein) is a Type 2 calreticulin protein (e.g., a wild-type or mutant calreticulin protein). In some embodiments, the first calreticulin protein comprises the amino acid sequence of SEQ ID NO: 6287, and the second calreticulin protein the amino acid sequence of SEQ ID NO: 6288. In some embodiments, the first calreticulin protein comprises the amino acid sequence of SEQ ID NO: 6313, and the first calreticulin protein comprises the amino acid sequence of SEQ ID NO: 6314.

In some embodiments, the wild type calreticulin protein comprises the amino acid sequence of SEQ ID NO: 6285 or 1001.

In some embodiments, the first antigen binding domain has about the same affinity (e.g., equal affinity) for the first calreticulin protein (e.g., a mutant calreticulin protein) and for a wild-type calreticulin protein.

In some embodiments, the second antigen binding domain has about the same affinity (e.g., equal affinity) for the second calreticulin protein (e.g., a mutant calreticulin protein) and for a wild-type calreticulin protein.

In some embodiments, the first antigen binding domain has a higher affinity for a first calreticulin mutant protein than for the wild type calreticulin protein. In some embodiments, the K_{D} for the binding between the first antigen binding domain and the first calreticulin mutant protein is no more than 40%, 30%, 20%, 10%, 1%, 0.1%, or 0.01% of the K_{D} for the binding between the first antigen binding domain and the wild type calreticulin protein. In some embodiments, the first antigen binding domain binds to an epitope located within the C-terminus of the first calreticulin mutant protein. In some embodiments, the first antigen binding domain binds to an epitope located within the amino acid sequence of SEQ ID NO: 6286. In some embodiments, the first antigen binding domain does not bind to the wild type calreticulin protein. In some embodiments, the wild type calreticulin protein comprises the amino acid sequence of SEQ ID NO: 6285 or 1001.

In some embodiments, the second antigen binding domain has a higher affinity for a second calreticulin mutant protein than for the wild type calreticulin protein. In some embodiments, the K_{D} for the binding between the second antigen binding domain and the second calreticulin mutant protein is no more than 40%, 30%, 20%, 10%, 1%, 0.1%, or 0.01% of the K_{D} for the binding between the second antigen binding domain and the wild type calreticulin protein. In some embodiments, the second antigen binding domain binds to an epitope located within the C-terminus of the second calreticulin mutant protein. In some embodiments, the second antigen binding domain binds to an epitope located within the amino acid sequence of SEQ ID NO: 6286. In some embodiments, the second antigen binding domain does not bind to the wild type calreticulin protein. In some embodiments, the wild type calreticulin protein comprises the amino acid sequence of SEQ ID NO: 6285 or 1001.

In some embodiments, the multifunctional molecule preferentially binds to a myeloproliferative neoplasm cell over a non-tumor cell. In some embodiments, the binding between the multifunctional molecule and the myeloproliferative neoplasm cell is more than 10, 20, 30, 40, 50-fold greater than the binding between the multifunctional molecule and a non-tumor cell. In some embodiments, the myeloproliferative neoplasm cell is chosen from a myelofibrosis cell, an essential thrombocythemia cell, a polycythemia vera cell, or a chronic myeloid cancer cell. In some embodiments, the myeloproliferative neoplasm cell does not comprise a JAK2 V617F mutation. In some embodiments, the myeloproliferative neoplasm cell does not comprise an MPL mutation.

In some embodiments, the first and/or second antigen binding domain comprises a heavy chain variable region (VH) comprising a heavy chain complementarity determining region 1 (VHCDR1) amino acid sequence of SEQ ID NO: 6253 (or a sequence with no more than 1, 2, 3, or 4 mutations, e.g., substitutions, additions, or deletions), a VHCDR2 amino acid sequence of SEQ ID NO: 6254 (or a sequence with no more than 1, 2, 3, or 4 mutations, e.g., substitutions, additions, or deletions), and/or a VHCDR3 amino acid sequence of SEQ ID NO: 6255 (or a sequence with no more than 1, 2, 3, or 4 mutations, e.g., substitutions, additions, or deletions). In some embodiments, the first and/or second antigen binding domain comprises a light chain variable region (VL) comprising a light chain complementarity determining region 1 (VLCDR1) amino acid sequence of SEQ ID NO: 6259 (or a sequence with no more than 1, 2, 3, or 4 mutations, e.g., substitutions, additions, or deletions), a VLCDR2 amino acid sequence of SEQ ID NO: 6260 (or a sequence with no more than 1, 2, 3, or 4 mutations, e.g., substitutions, additions, or deletions), and/or a VLCDR3 amino acid sequence of SEQ ID NO: 6261 (or a sequence with no more than 1, 2, 3, or 4 mutations, e.g., substitutions, additions, or deletions).

In some embodiments, the first and/or second antigen binding domain comprises:
(i) a heavy chain variable region (VH) comprising a heavy chain complementarity determining region 1 (VHCDR1) amino acid sequence of SEQ ID NO: 6253 (or a sequence with no more than 1, 2, 3, or 4 mutations, e.g., substitutions, additions, or deletions), a VHCDR2 amino acid sequence of SEQ ID NO: 6254 (or a sequence with no more than 1, 2, 3, or 4 mutations, e.g., substitutions, additions, or deletions), and/or a VHCDR3 amino acid sequence of SEQ ID NO: 6255 (or a sequence with no more than 1, 2, 3, or 4 mutations, e.g., substitutions, additions, or deletions), and
(ii) a light chain variable region (VL) comprising a light chain complementarity determining region 1 (VLCDR1) amino acid sequence of SEQ ID NO: 6259 (or a sequence with no more than 1, 2, 3, or 4 mutations, e.g., substitutions, additions, or deletions), a VLCDR2 amino acid sequence of SEQ ID NO: 6260 (or a sequence with no more than 1, 2, 3, or 4 mutations, e.g., substitutions, additions, or deletions), and/or a VLCDR3 amino acid sequence of SEQ ID NO: 6261 (or a sequence with no more than 1, 2, 3, or 4 mutations, e.g., substitutions, additions, or deletions).

In some embodiments, the first and/or second antigen binding domain comprises a VH comprising a VHCDR1 amino acid sequence of SEQ ID NO: 6253, a VHCDR2 amino acid sequence of SEQ ID NO: 6254, and a VHCDR3 amino acid sequence of SEQ ID NO: 6255. In some embodiments, the first and/or second antigen binding domain comprises a VL comprising a VLCDR1 amino acid sequence of SEQ ID NO: 6259, a VLCDR2 amino acid sequence of SEQ ID NO: 6260, and a VLCDR3 amino acid sequence of SEQ ID NO: 6261.

In some embodiments, the first and/or second antigen binding domain comprises:
(i) a VH comprising a VHCDR1 amino acid sequence of SEQ ID NO: 6253, a VHCDR2 amino acid sequence of SEQ ID NO: 6254, and a VHCDR3 amino acid sequence of SEQ ID NO: 6255, and
(ii) a VL comprising a VLCDR1 amino acid sequence of SEQ ID NO: 6259, a VLCDR2 amino acid sequence of SEQ ID NO: 6260, and a VLCDR3 amino acid sequence of SEQ ID NO: 6261.

In some embodiments, the first and/or second antigen binding domain comprises a VH comprising a heavy chain framework region 1 (VHFWR1) amino acid sequence of SEQ ID NO: 6224, a VHFWR2 amino acid sequence of SEQ ID NO: 6226, a VHFWR3 amino acid sequence of SEQ ID NO: 6228, and/or a VHFWR4 amino acid sequence of SEQ ID NO: 6230. In some embodiments, the first and/or second antigen binding domain comprises a VL comprising a light chain framework region 1 (VLFWR1) amino acid sequence of SEQ ID NO: 6238, a VLFWR2 amino acid sequence of SEQ ID NO: 6240, a VLFWR3 amino acid sequence of SEQ ID NO: 6242, and/or a VLFWR4 amino acid sequence of SEQ ID NO: 6244.

In some embodiments, the first and/or second antigen binding domain comprises:
(i) a VH comprising a heavy chain framework region 1 (VHFWR1) amino acid sequence of SEQ ID NO: 6224, a VHFWR2 amino acid sequence of SEQ ID NO: 6226, a VHFWR3 amino acid sequence of SEQ ID NO: 6228, and/or a VHFWR4 amino acid sequence of SEQ ID NO: 6230, and
(ii) a VL comprising a light chain framework region 1 (VLFWR1) amino acid sequence of SEQ ID NO: 6238, a VLFWR2 amino acid sequence of SEQ ID NO: 6240, a VLFWR3 amino acid sequence of SEQ ID NO: 6242, and/or a VLFWR4 amino acid sequence of SEQ ID NO: 6244.

In some embodiments, the first and/or second antigen binding domain comprises a VH comprising a VHFWR1 amino acid sequence of SEQ ID NO: 6263 (or a sequence with no more than 1, 2, 3, 4, 5, or 6 mutations, e.g., substitutions, additions, or deletions), a VHFWR2 amino acid sequence of SEQ ID NO: 6264 (or a sequence with no more than 1, 2, 3, 4, 5, or 6 mutations, e.g., substitutions, additions, or deletions), a VHFWR3 amino acid sequence of SEQ ID NO: 6265 (or a sequence with no more than 1, 2, 3, 4, 5, 6, 7, 8, 9, 10, or 11 mutations, e.g., substitutions, additions, or deletions), and/or a VHFWR4 amino acid sequence of SEQ ID NO: 228. In some embodiments, the first and/or second antigen binding domain comprises a VL comprising a VLFWR1 amino acid sequence of SEQ ID NO: 6277 (or a sequence with no more than 1, 2, or 3 mutations, e.g., substitutions, additions, or deletions), a VLFWR2 amino acid sequence of SEQ ID NO: 6278 (or a sequence with no more than 1 mutation, e.g., substitution, addition, or deletion), a VLFWR3 amino acid sequence of SEQ ID NO: 6279 (or a sequence with no more than 1 mutation, e.g., substitution, addition, or deletion), and/or a VLFWR4 amino acid sequence of SEQ ID NO: 6280.

In some embodiments, the first and/or second antigen binding domain comprises:
(i) a VH comprising a VHFWR1 amino acid sequence of SEQ ID NO: 6263 (or a sequence with no more than 1, 2, 3, 4, 5, or 6 mutations, e.g., substitutions, additions, or deletions), a VHFWR2 amino acid sequence of SEQ ID NO: 6264 (or a sequence with no more than 1, 2, 3, 4, 5, or 6 mutations, e.g., substitutions, additions, or deletions), a VHFWR3 amino acid sequence of SEQ ID NO: 6265 (or a sequence with no more than 1, 2, 3, 4, 5, 6, 7, 8, 9, 10, or 11 mutations, e.g., substitutions, additions, or deletions), and/or a VHFWR4 amino acid sequence of SEQ ID NO: 228, and
(ii) a VL comprising a VLFWR1 amino acid sequence of SEQ ID NO: 6277 (or a sequence with no more than 1, 2, or 3 mutations, e.g., substitutions, additions, or deletions), a VLFWR2 amino acid sequence of SEQ ID NO: 6278 (or a sequence with no more than 1 mutation, e.g., substitution, addition, or deletion), a VLFWR3 amino acid sequence of SEQ ID NO: 6279 (or a sequence with no more than 1 mutation, e.g., substitution, addition, or deletion), and/or a VLFWR4 amino acid sequence of SEQ ID NO: 6280.

In some embodiments, the first and/or second antigen binding domain comprises a VH comprising a VHFWR1 amino acid sequence of SEQ ID NO: 6263, a VHFWR2 amino acid sequence of SEQ ID NO: 6264, a VHFWR3 amino acid sequence of SEQ ID NO: 6265, and/or a VHFWR4 amino acid sequence of SEQ ID NO: 228. In some embodiments, the first and/or second antigen binding domain comprises a VL comprising a VLFWR1 amino acid sequence of SEQ ID NO: 6277, a VLFWR2 amino acid sequence of SEQ ID NO: 6278, a VLFWR3 amino acid sequence of SEQ ID NO: 6279, and/or a VLFWR4 amino acid sequence of SEQ ID NO: 6280.

In some embodiments, the first and/or second antigen binding domain comprises:
(i) a VH comprising a VHFWR1 amino acid sequence of SEQ ID NO: 6263, a VHFWR2 amino acid sequence of SEQ ID NO: 6264, a VHFWR3 amino acid sequence of SEQ ID NO: 6265, and/or a VHFWR4 amino acid sequence of SEQ ID NO: 228, and
(ii) a VL comprising a VLFWR1 amino acid sequence of SEQ ID NO: 6277, a VLFWR2 amino acid sequence of SEQ ID NO: 6278, a VLFWR3 amino acid sequence of SEQ ID NO: 6279, and/or a VLFWR4 amino acid sequence of SEQ ID NO: 6280.

In some embodiments, the first and/or second antigen binding domain comprises a VH comprising the amino acid sequence of SEQ ID NO: 6247 (or an amino acid sequence having at least about 77%, 80%, 85%, 90%, 95%, or 99% sequence identity to SEQ ID NO: 6247). In some embodiments, the first and/or second antigen binding domain comprises a VL comprising the amino acid sequence of SEQ ID NO: 6249 (or an amino acid sequence having at least about 93%, 95%, or 99% sequence identity to SEQ ID NO: 6249).

In some embodiments, the first and/or second antigen binding domain comprises:
(i) a VH comprising the amino acid sequence of SEQ ID NO: 6247 (or an amino acid sequence having at least about 77%, 80%, 85%, 90%, 95%, or 99% sequence identity to SEQ ID NO: 6247), and
(ii) a VL comprising the amino acid sequence of SEQ ID NO: 6249 (or an amino acid sequence having at least about 93%, 95%, or 99% sequence identity to SEQ ID NO: 6249).

In some embodiments, the first and/or second antigen binding domain comprises a VH comprising the amino acid sequence of SEQ ID NO: 6247. In some embodiments, the first and/or second antigen binding domain comprises a VL comprising the amino acid sequence of SEQ ID NO: 6249. In some embodiments, the first and/or second antigen binding domain comprises (i) a VH comprising the amino acid sequence of SEQ ID NO: 6247, and (ii) a VL comprising the amino acid sequence of SEQ ID NO: 6249.

In some embodiments, the first and/or second antigen binding domain comprises a VH comprising an amino acid sequence of at least 70% or 75% sequence identity to SEQ ID NO: 6250. In some embodiments, the first and/or second antigen binding domain comprises a VL comprising an amino acid sequence of at least 85% or 90% sequence identity to SEQ ID NO: 6252. In some embodiments, the first and/or second antigen binding domain comprises (i) a VH comprising an amino acid sequence of at least 70% or 75% sequence identity to SEQ ID NO: 6250, and (ii) a VL comprising an amino acid sequence of at least 85% or 90% sequence identity to SEQ ID NO: 6252.

In some embodiments, the first and/or second antigen binding domain comprises a heavy chain variable region (VH) comprising a heavy chain complementarity determining region 1 (VHCDR1) amino acid sequence of SEQ ID NO: 6256 (or a sequence with no more than 1, 2, 3, or 4 mutations, e.g., substitutions, additions, or deletions), a VHCDR2 amino acid sequence of SEQ ID NO: 6257 (or a sequence with no more than 1, 2, 3, or 4 mutations, e.g., substitutions, additions, or deletions), and/or a VHCDR3 amino acid sequence of SEQ ID NO: 6258 or 116 (or a sequence with no more than 1, 2, 3, or 4 mutations, e.g., substitutions, additions, or deletions). In some embodiments, the first and/or second antigen binding domain comprises a light chain variable region (VL) comprising a light chain complementarity determining region 1 (VLCDR1) amino acid sequence of SEQ ID NO: 6259 (or a sequence with no more than 1, 2, 3, or 4 mutations, e.g., substitutions, additions, or deletions), a VLCDR2 amino acid sequence of SEQ ID NO: 6260 (or a sequence with no more than 1, 2, 3, or 4 mutations, e.g., substitutions, additions, or deletions), and/or a VLCDR3 amino acid sequence of SEQ ID NO: 6261 (or a sequence with no more than 1, 2, 3, or 4 mutations, e.g., substitutions, additions, or deletions).

In some embodiments, the first and/or second antigen binding domain comprises:
(i) a heavy chain variable region (VH) comprising a heavy chain complementarity determining region 1 (VHCDR1) amino acid sequence of SEQ ID NO: 6256 (or a sequence with no more than 1, 2, 3, or 4 mutations, e.g., substitutions, additions, or deletions), a VHCDR2 amino acid sequence of SEQ ID NO: 6257 (or a sequence with no more than 1, 2, 3, or 4 mutations, e.g., substitutions, additions, or deletions), and/or a VHCDR3 amino acid sequence of SEQ ID NO: 6258 or 116 (or a sequence with no more than 1, 2, 3, or 4 mutations, e.g., substitutions, additions, or deletions), and
(ii) a light chain variable region (VL) comprising a light chain complementarity determining region 1 (VLCDR1) amino acid sequence of SEQ ID NO: 6259 (or a sequence with no more than 1, 2, 3, or 4 mutations, e.g., substitutions, additions, or deletions), a VLCDR2 amino acid sequence of SEQ ID NO: 6260 (or a sequence with no more than 1, 2, 3, or 4 mutations, e.g., substitutions, additions, or deletions), and/or a VLCDR3 amino acid sequence of SEQ ID NO: 6261 (or a sequence with no more than 1, 2, 3, or 4 mutations, e.g., substitutions, additions, or deletions).

In some embodiments, the first and/or second antigen binding domain comprises a VH comprising a heavy chain framework region 1 (VHFWR1) amino acid sequence of SEQ ID NO: 6232, a VHFWR2 amino acid sequence of SEQ ID NO: 6234, a VHFWR3 amino acid sequence of SEQ ID NO: 6236, and/or a VHFWR4 amino acid sequence of SEQ ID NO: 6230. In some embodiments, the first and/or second antigen binding domain comprises a VL comprising a light chain framework region 1 (VLFWR1) amino acid sequence of SEQ ID NO: 6238, a VLFWR2 amino acid sequence of SEQ ID NO: 6240, a VLFWR3 amino acid sequence of SEQ ID NO: 6242, and/or a VLFWR4 amino acid sequence of SEQ ID NO: 6244.

In some embodiments, the first and/or second antigen binding domain comprises:
(i) a VH comprising a heavy chain framework region 1 (VHFWR1) amino acid sequence of SEQ ID NO: 6232, a VHFWR2 amino acid sequence of SEQ ID NO: 6234, a VHFWR3 amino acid sequence of SEQ ID NO: 6236, and/or a VHFWR4 amino acid sequence of SEQ ID NO: 6230, and
(ii) a VL comprising a light chain framework region 1 (VLFWR1) amino acid sequence of SEQ ID NO: 6238, a VLFWR2 amino acid sequence of SEQ ID NO: 6240, a VLFWR3 amino acid sequence of SEQ ID NO: 6242, and/or a VLFWR4 amino acid sequence of SEQ ID NO: 6244.

In some embodiments, the first and/or second antigen binding domain comprises a VH comprising a heavy chain framework 1 (VHFWR1) amino acid sequence of SEQ ID NO: 6266 (or a sequence with no more than 1, 2, 3, 4, 5, 6, 7, 8, or 9 mutations, e.g., substitutions, additions, or deletions), a VHFWR2 amino acid sequence of SEQ ID NO: 6267 (or a sequence with no more than 1, 2, 3, or 4 mutations, e.g., substitutions, additions, or deletions), a VHFWR3 amino acid sequence of SEQ ID NO: 6268 (or a sequence with no more than 1, 2, 3, 4, 5, 6, 7, 8, 9, 10, or 11 mutations, e.g., substitutions, additions, or deletions), and/or a VHFWR4 amino acid sequence of SEQ ID NO: 6269. In some embodiments, the first and/or second antigen binding domain comprises a VL comprising a VLFWR1 amino acid sequence of SEQ ID NO: 6277 (or a sequence with no more than 1, 2, or 3 mutations, e.g., substitutions, additions, or deletions), a VLFWR2 amino acid sequence of SEQ ID NO: 6278 (or a sequence with no more than 1 mutation, e.g., substitution, addition, or deletion), a VLFWR3 amino acid sequence of SEQ ID NO: 6279 (or a sequence with no more than 1 mutation, e.g., substitution, addition, or deletion), and/or a VLFWR4 amino acid sequence of SEQ ID NO: 6280.

In some embodiments, the first and/or second antigen binding domain comprises:
(i) a VH comprising a heavy chain framework 1 (VHFWR1) amino acid sequence of SEQ ID NO: 6266 (or a sequence with no more than 1, 2, 3, 4, 5, 6, 7, 8, or 9 mutations, e.g., substitutions, additions, or deletions), a VHFWR2 amino acid sequence of SEQ ID NO: 6267 (or a sequence with no more than 1, 2, 3, or 4 mutations, e.g., substitutions, additions, or deletions), a VHFWR3 amino acid sequence of SEQ ID NO: 6268 (or a sequence with no more than 1, 2, 3, 4, 5, 6, 7, 8, 9, 10, or 11 mutations, e.g., substitutions, additions, or deletions), and/or a VHFWR4 amino acid sequence of SEQ ID NO: 6269, and
(ii) a VL comprising a VLFWR1 amino acid sequence of SEQ ID NO: 6277 (or a sequence with no more than 1, 2, or 3 mutations, e.g., substitutions, additions, or deletions), a VLFWR2 amino acid sequence of SEQ ID NO: 6278 (or a sequence with no more than 1 mutation, e.g., substitution, addition, or deletion), a VLFWR3 amino acid sequence of SEQ ID NO: 6279 (or a sequence with no more than 1 mutation, e.g., substitution, addition, or deletion), and/or a VLFWR4 amino acid sequence of SEQ ID NO: 6280.

In some embodiments, the first and/or second antigen binding domain comprises a VH comprising the amino acid sequence of SEQ ID NO: 6248 (or an amino acid sequence having at least about 75%, 80%, 85%, 90%, 95%, or 99% sequence identity to SEQ ID NO: 6248). In some embodiments, the first and/or second antigen binding domain comprises a VL comprising the amino acid sequence of SEQ ID NO: 6249 (or an amino acid sequence having at least about 93%, 95%, or 99% sequence identity to SEQ ID NO: 6249).

In some embodiments, the first and/or second antigen binding domain comprises
(i) a VH comprising the amino acid sequence of SEQ ID NO: 6248 (or an amino acid sequence having at least about 75%, 80%, 85%, 90%, 95%, or 99% sequence identity to SEQ ID NO: 6248), and
(ii) a VL comprising the amino acid sequence of SEQ ID NO: 6249 (or an amino acid sequence having at least about 93%, 95%, or 99% sequence identity to SEQ ID NO: 6249).

In some embodiments, the first and/or second antigen binding domain comprises a VH comprising the amino acid sequence of SEQ ID NO: 6248. In some embodiments, the first and/or second antigen binding domain comprises a VL comprising the amino acid sequence of SEQ ID NO: 6249. In some embodiments, the first and/or second antigen binding domain comprises (i) a VH comprising the amino acid sequence of SEQ ID NO: 6248, and (ii) a VL comprising the amino acid sequence of SEQ ID NO: 6249.

In some embodiments, the first and/or second antigen binding domain comprises a VH comprising an amino acid sequence of at least 70% or 74% sequence identity to SEQ ID NO: 6251. In some embodiments, the first and/or second antigen binding domain comprises a VL comprising an amino acid sequence of at least 85% or 90% sequence identity to SEQ ID NO: 6252. In some embodiments, the first and/or second antigen binding domain comprises (i) a VH comprising an amino acid sequence of at least 70% or 74% sequence identity to SEQ ID NO: 6251, and/or (ii) a VL comprising an amino acid sequence of at least 85% or 90% sequence identity to SEQ ID NO: 6252.

In some embodiments, the multifunctional molecule comprises an immune cell engager chosen from a T cell engager, an NK cell engager, a B cell engager, a dendritic cell engager, or a macrophage cell engager. In some embodiments, the immune cell engager binds to and activates an immune cell, e.g., an effector cell. In some embodiments, the immune cell engager binds to, but does not activate, an immune cell, e.g., an effector cell.

In some embodiments, the immune cell engager is a T cell engager, e.g., a T cell engager that mediates binding to and activation of a T cell, or a T cell engager that mediates binding to but not activation of a T cell. In some embodiments, the T cell engager binds to CD3, TCRα, TCRβ, TCRγ, TCRξ, ICOS, CD28, CD27, HVEM, LIGHT, CD40, 4-1BB, OX40, DR3, GITR, CD30, TIM1, SLAM, CD2, or CD226. In some embodiments, the T cell engager is an anti-CD3 antibody molecule. In some embodiments, the T cell engager is an anti-TCRβ antibody molecule, e.g., an anti-TCRβV antibody molecule described herein.

In some embodiments, the immune cell engager is an NK cell engager, e.g., an NK cell engager that mediates binding to and activation of an NK cell, or an NK cell engager that mediates binding to but not activation of an NK cell. In some embodiments, the NK cell engager is chosen from an antibody molecule, e.g., an antigen binding domain, or ligand that binds to (e.g., activates): NKp30, NKp40, NKp44, NKp46, NKG2D, DNAM1, DAP10, CD16 (e.g., CD16a, CD16b, or both), CRTAM, CD27, PSGL1, CD96, CD100 (SEMA4D), NKp80, CD244 (also known as SLAMF4 or 2B4), SLAMF6, SLAMF7, KIR2DS2, KIR2DS4, KIR3DS1, KIR2DS3, KIR2DS5, KIR2DS1, CD94, NKG2C, NKG2E, or CD160. In some embodiments, the NK cell engager is an antibody molecule or ligand that binds to (e.g., activates) NKp30. In some embodiments, the NK cell engager is an antibody molecule, e.g., an antigen binding domain. In some embodiments, the NK cell engager is an antibody molecule, e.g., an antigen binding domain, that binds to NKp30 or NKp46. In some embodiments, the NK cell engager is a ligand, optionally, the ligand further comprises an immunoglobulin constant region, e.g., an Fc region. In some embodiments, the NK cell engager is a ligand of NKp44 or NKp46, e.g., a viral HA. In some embodiments, the NK cell engager is a ligand of DAP10, e.g., a coreceptor for NKG2D. In some embodiments, the NK cell engager is a ligand of CD16, e.g., a CD16a/b ligand, e.g., a CD16a/b ligand further comprising an antibody Fc region. In some embodiments, the immune cell engager mediates binding to, or activation of, or both of, one or more of a B cell, a macrophage, and/or a dendritic cell.

In some embodiments, the immune cell engager comprises a B cell, macrophage, and/or dendritic cell engager chosen from one or more of CD40 ligand (CD40L) or a CD70 ligand; an antibody molecule that binds to CD40 or CD70; an antibody molecule to OX40; an OX40 ligand (OX40L); an agonist of a Toll-like receptor (e.g., a TLR4, e.g., a constitutively active TLR4 (caTLR4) or a TLR9 agonist); a 41BB; a CD2 agonist; a CD47; or a STING agonist, or a combination thereof. In some embodiments, the immune cell engager is a B cell engager, e.g., a CD40L, an OX40L, or a CD70 ligand, or an antibody molecule that binds to OX40, CD40 or CD70. In some embodiments, the immune cell engager is a macrophage cell engager, e.g., a CD2 agonist; a CD40L; an OX40L; an antibody molecule that binds to OX40, CD40 or CD70; an agonist of a Toll-like receptor (TLR) (e.g., a TLR4, e.g., a constitutively active TLR4 (caTLR4) or a TLR9 agonist); CD47; or a STING agonist. In some embodiments, the immune cell engager is a dendritic cell engager, e.g., a CD2 agonist, an OX40 antibody, an OX40L, 41BB agonist, a Toll-like receptor agonist or a fragment thereof (e.g., a TLR4, e.g., a constitutively active TLR4 (caTLR4)), CD47 agonist, or a STING agonist. In some embodiments, the STING agonist comprises a cyclic dinucleotide, e.g., a cyclic di-GMP (cdGMP), a cyclic di-AMP (cdAMP), or a combination thereof, optionally with 2′,5′ or 3′,5′ phosphate linkages, e.g., wherein the STING agonist is covalently coupled to the multifunctional molecule.

In some embodiments, the multifunctional molecule comprises a cytokine molecule or a modulator thereof. In some embodiments, the cytokine molecule is chosen from TGF-β, interleukin-2 (IL-2), interleukin-7 (IL-7), interleukin-12 (IL-12), interleukin-15 (IL-15), interleukin-18 (IL-18), interleukin-21 (IL-21), or interferon gamma, or a fragment or variant thereof, or a combination of any of the aforesaid cytokines. In some embodiments, the cytokine molecule is a monomer or a dimer. In some embodiments, the cytokine molecule further comprises a receptor dimerizing domain, e.g., an IL15Ralpha dimerizing domain. In some embodiments, the cytokine molecule (e.g., IL-15) and the receptor dimerizing domain (e.g., an IL15Ralpha dimerizing domain) are not covalently linked, e.g., are non-covalently associated.

In some embodiments, the modulator of the cytokine molecule comprises a TGF-β inhibitor.

In some embodiments, the multifunctional molecule comprises a stromal modifying moiety. In some embodiments, the stromal modifying moiety causes one or more of: decreases the level or production of a stromal or extracellular matrix (ECM) component; decreases tumor fibrosis; increases interstitial tumor transport; improves tumor perfusion; expands the tumor microvasculature; decreases interstitial fluid pressure (IFP) in a tumor; or decreases or enhances penetration or diffusion of an agent, e.g., a cancer therapeutic or a cellular therapy, into a tumor or tumor vasculature. In some embodiments, the stromal or ECM component decreased is chosen from a glycosaminoglycan or an extracellular protein, or a combination thereof. In some embodiments, the glycosaminoglycan is chosen from hyaluronan (also known as hyaluronic acid or HA), chondroitin sulfate, chondroitin, dermatan sulfate, heparan sulfate, heparin, entactin, tenascin, aggrecan or keratin sulfate. In some embodiments, the extracellular protein is chosen from collagen, laminin, elastin, fibrinogen, fibronectin, or vitronectin. In some embodiments, the stromal modifying moiety comprises an enzyme molecule that degrades a tumor stroma or extracellular matrix (ECM). In some embodiments, the enzyme molecule is chosen from a hyaluronidase molecule, a collagenase molecule, a chondroitinase molecule, a matrix metalloproteinase molecule (e.g., macrophage metalloelastase), or a variant (e.g., a fragment) of any of the aforesaid. In some embodiments, the stromal modifying moiety decreases the level or production of hyaluronic acid. In some embodiments, the stromal modifying moiety comprises a hyaluronan degrading enzyme, an agent that inhibits hyaluronan synthesis, or an antibody molecule against hyaluronic acid. In some embodiments, the hyaluronan degrading enzyme is a hyaluronidase molecule or a variant (e.g., fragment thereof) thereof. In some embodiments, the hyaluronan degrading enzyme is active in neutral or acidic pH, e.g., pH of about 4-5. In some embodiments, the hyaluronidase molecule is a mammalian hyaluronidase molecule, e.g., a recombinant human hyaluronidase molecule, or a variant thereof (e.g., a truncated form thereof). In some embodiments, the hyaluronidase molecule is chosen from HYAL1, HYAL2, or PH-20/SPAM1, or a variant thereof (e.g., a truncated form thereof). In some embodiments, the truncated form lacks a C-terminal glycosylphosphatidylinositol (GPI) attachment site or a portion of the GPI attachment site. In some embodiments, the hyaluronidase molecule is glycosylated, e.g., comprises at least one N-linked glycan. In some embodiments, the hyaluronidase molecule comprises the amino acid sequence of SEQ ID NO: 6213, or a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 6213). In some embodiments, the hyaluronidase molecule comprises the amino acid residues 36-464 of SEQ ID NO: 6213. In some embodiments, the hyaluronidase molecule comprises the amino acid residues 36-481, 36-482, or 36-483 of PH20, wherein PH20 has the amino acid sequence of SEQ ID NO: 6213. In some embodiments, the hyaluronidase molecule comprises an amino acid sequence having at least 95% to 100% sequence identity to the polypeptide or truncated form of the amino acid sequence of SEQ ID NO: 6213. In some embodiments, the hyaluronidase molecule comprises an amino acid sequence having 30, 20, 10, 5 or fewer amino acid substitutions to the amino acid sequence of SEQ ID NO: 6213. In some embodiments, the hyaluronidase molecule comprises an amino acid sequence at least 95% (e.g., at least 95%, 96%, 97%, 98%, 99%, 100%) identical to the amino acid sequence of SEQ ID NO: 6213. In some embodiments, the hyaluronidase molecule is encoded by a nucleotide sequence at least 95% (e.g., at least 96%, 97%, 98%, 99%, 100%) identical to the nucleotide sequence of SEQ ID NO: 6213. In some embodiments, the hyaluronidase molecule is PH20, e.g., rHuPH20. In some embodiments, the hyaluronidase molecule is HYAL1 and comprises the amino acid sequence of SEQ ID NO: 6218, or a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 6218). In some embodiments, the hyaluronan degrading enzyme, e.g., the hyaluronidase molecule, further comprises a polymer, e.g., is conjugated to a polymer, e.g., PEG. In some embodiments, the hyaluronan-degrading enzyme is a PEGylated PH20 enzyme (PEGPH20). In some embodiments, the hyaluronan degrading enzyme, e.g., the hyaluronidase molecule, further comprises an immunoglobulin chain constant region (e.g., Fc region) chosen from, e.g., the heavy chain constant regions of IgG1, IgG2, IgG3, or IgG4, more particularly, the heavy chain constant region of human IgG1, IgG2, IgG3, or IgG4. In some embodiments, the immunoglobulin constant region (e.g., the Fc region) is linked, e.g., covalently linked to, the hyaluronan degrading enzyme, e.g., the hyaluronidase molecule. In some embodiments, the immunoglobulin chain constant region (e.g., Fc region) is altered, e.g., mutated, to increase or decrease one or more of: Fc receptor binding, antibody glycosylation, the number of cysteine residues, effector cell function, or complement function. In some embodiments, the hyaluronan degrading enzyme, e.g., the hyaluronidase molecule, forms a dimer. In some embodiments, the stromal modifying moiety comprises an inhibitor of the synthesis of hyaluronan, e.g., an HA synthase. In some embodiments, the inhibitor comprises a sense or an antisense nucleic acid molecule against an HA synthase or is a small molecule drug. In some embodiments, the inhibitor is 4-methylumbelliferone (MU) or a derivative thereof (e.g., 6,7-dihydroxy-4-methyl coumarin or 5,7-dihydroxy-4-methyl coumarin), or leflunomide or a derivative thereof. In some embodiments, the stromal modifying moiety comprises a collagenase molecule, e.g., a mammalian collagenase molecule, or a variant (e.g., fragment) thereof. In some embodiments, the collagenase molecule is collagenase molecule IV, e.g., comprising the amino acid sequence of SEQ ID NO: 6219, or a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 6219.

In some embodiments, the multifunctional molecule comprises an immune cell engager (e.g., a T cell engager, an NK cell engager, a B cell engager, a dendritic cell engager, or a macrophage cell engager) and a cytokine molecule. In some embodiments, the multifunctional molecule comprises an immune cell engager (e.g., a T cell engager, an NK cell engager, a B cell engager, a dendritic cell engager, or a macrophage cell engager) and a stromal modifying moiety. In some embodiments, the multifunctional molecule comprises a cytokine molecule and a stromal modifying moiety. In some embodiments, the multifunctional molecule comprises an immune cell engager (e.g., a T cell engager, an NK cell engager, a B cell engager, a dendritic cell engager, or a macrophage cell engager), a cytokine molecule, and a stromal modifying moiety.

In some embodiments, the multifunctional molecule comprises at least two non-contiguous polypeptide chains.

In some embodiments, the multifunctional molecule comprises the following configuration:
A, B-[dimerization module]-C, -D
   e.g., the configuration shown in FIGS. 1A, 1B, and 1C, wherein:
   (1) the dimerization module comprises an immunoglobulin constant domain, e.g., a heavy chain constant domain (e.g., a homodimeric or heterodimeric heavy chain constant region, e.g., an Fc region), or a constant domain of an immunoglobulin variable region (e.g., a Fab region); and
   (2) A, B, C, and D are independently absent; (i) an antigen binding domain that binds to a calreticulin protein (e.g., a wild type calreticulin protein or a mutant calreticulin protein), wherein the calreticulin protein comprises the amino acid sequence of SEQ ID NO: 6286; (ii) an immune cell engager chosen from a T cell engager, an NK cell engager, a B cell engager, a dendritic cell engager, or a macrophage cell engager; (iii) a cytokine molecule; or (iv) a stromal modifying moiety, provided that: at least one, two, or three of A, B, C, and D comprises an antigen binding domain that binds to a calreticulin protein (e.g., a wild type calreticulin protein or a calreticulin mutant protein), wherein the calreticulin protein comprises the amino acid sequence of SEQ ID NO: 6286, and any of the remaining A, B, C, and D is absent or comprises one of an immune cell engager, a cytokine molecule, or a stromal modifying moiety.

In some embodiments,
(i) A comprises an antigen binding domain that binds to a calreticulin protein (e.g., a wild type calreticulin protein or a calreticulin mutant protein), wherein the calreticulin protein comprises the amino acid sequence of SEQ ID NO: 6286, and B, C, or D comprises an immune cell engager, e.g., a T cell engager, e.g., an anti-CD3 antibody molecule;
(ii) A comprises an antigen binding domain that binds to a calreticulin protein (e.g., a wild type calreticulin protein or a calreticulin mutant protein), wherein the calreticulin protein comprises the amino acid sequence of SEQ ID NO: 6286, and B, C, or D comprises a cytokine molecule;
(iii) A comprises an antigen binding domain that binds to a calreticulin protein (e.g., a wild type calreticulin protein or a calreticulin mutant protein), wherein the calreticulin protein comprises the amino acid sequence of SEQ ID NO: 6286, and B, C, or D comprises a stromal modifying moiety;
(iv) A comprises a first antigen binding domain that binds to a first calreticulin protein (e.g., a wild type calreticulin protein or a calreticulin mutant protein), wherein the first calreticulin protein comprises the amino acid sequence of SEQ ID NO: 6286, B comprises a second antigen binding domain that binds to a second calreticulin protein (e.g., a wild type calreticulin protein or a calreticulin mutant protein), wherein the second calreticulin protein comprises the amino acid sequence of SEQ ID NO: 6286, and C or D comprises an immune cell engager, e.g., a T cell engager, e.g., an anti-CD3 antibody molecule;
(v) A comprises a first antigen binding domain that binds to a first calreticulin protein (e.g., a wild type calreticulin protein or a calreticulin mutant protein), wherein the first calreticulin protein comprises the amino acid sequence of SEQ ID NO: 6286, B comprises a second antigen binding domain that binds to a second calreticulin protein (e.g., a wild type calreticulin protein or a calreticulin mutant protein), wherein the second calreticulin protein comprises the amino acid sequence of SEQ ID NO: 6286, and C or D comprises a cytokine molecule;
(vi) A comprises a first antigen binding domain that binds to a first calreticulin protein (e.g., a wild type calreticulin protein or a calreticulin mutant protein), wherein the first calreticulin protein comprises the amino acid sequence of SEQ ID NO: 6286, B comprises a second antigen binding domain that binds to a second calreticulin protein (e.g., a wild type calreticulin protein or a calreticulin mutant protein), wherein the second calreticulin protein comprises the amino acid sequence of SEQ ID NO: 6286, and C or D comprises a stromal modifying moiety;
(vii) A comprises a first antigen binding domain that binds to a first calreticulin protein (e.g., a wild type calreticulin protein or a calreticulin mutant protein), wherein the first calreticulin protein comprises the amino acid sequence of SEQ ID NO: 6286, C comprises a second antigen binding domain that binds to a second calreticulin protein (e.g., a wild type calreticulin protein or a calreticulin mutant protein), wherein the second calreticulin protein comprises the amino acid sequence of SEQ ID NO: 6286, and B or D comprises an immune cell engager, e.g., a T cell engager, e.g., an anti-CD3 antibody molecule;
(viii) A comprises a first antigen binding domain that binds to a first calreticulin protein (e.g., a wild type calreticulin protein or a calreticulin mutant protein), wherein the first calreticulin protein comprises the amino acid sequence of SEQ ID NO: 6286, C comprises a second antigen binding domain that binds to a second calreticulin protein (e.g., a wild type calreticulin protein or a calreticulin mutant protein), wherein the second calreticulin protein comprises the amino acid sequence of SEQ ID NO: 6286, and B or D comprises a cytokine molecule;
(ix) A comprises a first antigen binding domain that binds to a first calreticulin protein (e.g., a wild type calreticulin protein or a calreticulin mutant protein), wherein the first calreticulin protein comprises the amino acid sequence of SEQ ID NO: 6286, C comprises a second antigen binding domain that binds to a second calreticulin protein (e.g., a wild type calreticulin protein or a calreticulin mutant protein), wherein the second calreticulin protein comprises the amino acid sequence of SEQ ID NO: 6286, and B or D comprises a stromal modifying moiety;
(x) A comprises a first antigen binding domain that binds to a first calreticulin protein (e.g., a wild type calreticulin protein or a calreticulin mutant protein), wherein the first calreticulin protein comprises the amino acid sequence of SEQ ID NO: 6286, and B, C, or D comprises (a) an immune cell engager, e.g., a T cell engager, e.g., an anti-CD3 antibody molecule and (b) a cytokine molecule;
(xi) A comprises a first antigen binding domain that binds to a first calreticulin protein (e.g., a wild type calreticulin protein or a calreticulin mutant protein), wherein the first calreticulin protein comprises the amino acid sequence of SEQ ID NO: 6286, and B, C, or D comprises (a) an immune cell engager, e.g., a T cell engager, e.g., an anti-CD3 antibody molecule and (b) a stromal modifying moiety;
(xii) A comprises a first antigen binding domain that binds to a first calreticulin protein (e.g., a wild type calreticulin protein or a calreticulin mutant protein), wherein the first calreticulin protein comprises the amino acid sequence of SEQ ID NO: 6286, and B, C, or D comprises (a) a cytokine molecule and (b) a stromal modifying moiety;
(xiii) A comprises a first antigen binding domain that binds to a first calreticulin protein (e.g., a wild type calreticulin protein or a calreticulin mutant protein), wherein the first calreticulin protein comprises the amino acid sequence of SEQ ID NO: 6286, B comprises a second antigen binding domain that binds to a second calreticulin protein (e.g., a wild type calreticulin protein or a calreticulin mutant protein), wherein the second calreticulin protein comprises the amino acid sequence of SEQ ID NO: 6286, and C or D comprises (a) an immune cell engager, e.g., a T cell engager, e.g., an anti-CD3 antibody molecule and (b) a cytokine molecule;
(xiv) A comprises a first antigen binding domain that binds to a first calreticulin protein (e.g., a wild type calreticulin protein or a calreticulin mutant protein), wherein the first calreticulin protein comprises the amino acid sequence of SEQ ID NO: 6286, B comprises a second antigen binding domain that binds to a second calreticulin protein (e.g., a wild type calreticulin protein or a calreticulin mutant protein), wherein the second calreticulin protein comprises the amino acid sequence of SEQ ID NO: 6286, and C or D comprises (a) an immune cell engager, e.g., a T cell engager, e.g., an anti-CD3 antibody molecule and (b) a stromal modifying moiety;
(xv) A comprises a first antigen binding domain that binds to a first calreticulin protein (e.g., a wild type calreticulin protein or a calreticulin mutant protein), wherein the first calreticulin protein comprises the amino acid sequence of SEQ ID NO: 6286, B comprises a second antigen binding domain that binds to a second calreticulin protein (e.g., a wild type calreticulin protein or a calreticulin mutant protein), wherein the second calreticulin protein comprises the amino acid sequence of SEQ ID NO: 6286, and C or D comprises (a) a cytokine molecule and (b) a stromal modifying moiety;
(xvi) A comprises a first antigen binding domain that binds to a first calreticulin protein (e.g., a wild type calreticulin protein or a calreticulin mutant protein), wherein the first calreticulin protein comprises the amino acid sequence of SEQ ID NO: 6286, C comprises a second antigen binding domain that binds to a second calreticulin protein (e.g., a wild type calreticulin protein or a calreticulin mutant protein), wherein the second calreticulin protein comprises the amino acid sequence of SEQ ID NO: 6286, and B or D comprises (a) an immune cell engager, e.g., a T cell engager, e.g., an anti-CD3 antibody molecule and (b) a cytokine molecule;
(xvii) A comprises a first antigen binding domain that binds to a first calreticulin protein (e.g., a wild type calreticulin protein or a calreticulin mutant protein), wherein the first calreticulin protein comprises the amino acid sequence of SEQ ID NO: 6286, C comprises a second antigen binding domain that binds to a second calreticulin protein (e.g., a wild type calreticulin protein or a calreticulin mutant protein), wherein the second calreticulin protein comprises the amino acid sequence of SEQ ID NO: 6286, and B or D comprises (a) an immune cell engager, e.g., a T cell engager, e.g., an anti-CD3 antibody molecule and (b) a stromal modifying moiety;
(xviii) A comprises a first antigen binding domain that binds to a first calreticulin protein (e.g., a wild type calreticulin protein or a calreticulin mutant protein), wherein the first calreticulin protein comprises the amino acid sequence of SEQ ID NO: 6286, C comprises a second antigen binding domain that binds to a second calreticulin protein (e.g., a wild type calreticulin protein or a calreticulin mutant protein), wherein the second calreticulin protein comprises the amino acid sequence of SEQ ID NO: 6286, and B or D comprises (a) a cytokine molecule and (b) a stromal modifying moiety;
(xix) A comprises a first antigen binding domain that binds to a first calreticulin protein (e.g., a wild type calreticulin protein or a calreticulin mutant protein), wherein the first calreticulin protein comprises the amino acid sequence of SEQ ID NO: 6286, and B, C, or D comprises (a) an immune cell engager, e.g., a T cell engager, e.g., an anti-CD3 antibody molecule, (b) a cytokine molecule, and (c) a stromal modifying moiety;
(xx) A comprises a first antigen binding domain that binds to a first calreticulin protein (e.g., a wild type calreticulin protein or a calreticulin mutant protein), wherein the first calreticulin protein comprises the amino acid sequence of SEQ ID NO: 6286, B comprises a second antigen binding domain that binds to a second calreticulin protein (e.g., a wild type calreticulin protein or a calreticulin mutant protein), wherein the second calreticulin protein comprises the amino acid sequence of SEQ ID NO: 6286, and C or D comprises (a) an immune cell engager, e.g., a T cell engager, e.g., an anti-CD3 antibody molecule, (b) a cytokine molecule, and (c) a stromal modifying moiety; or
(xxi) A comprises a first antigen binding domain that binds to a first calreticulin protein (e.g., a wild type calreticulin protein or a calreticulin mutant protein), wherein the first calreticulin protein comprises the amino acid sequence of SEQ ID NO: 6286, C comprises a second antigen binding domain that binds to a second calreticulin protein (e.g., a wild type calreticulin protein or a calreticulin mutant protein), wherein the second calreticulin protein comprises the amino acid sequence of SEQ ID NO: 6286, and B or D comprises (a) an immune cell engager, e.g., a T cell engager, e.g., an anti-CD3 antibody molecule, (b) a cytokine molecule, and (c) a stromal modifying moiety.

In some embodiments, the dimerization module comprises one or more immunoglobulin chain constant regions (e.g., Fc regions) comprising one or more of: a paired cavity-protuberance (“knob-in-a hole”), an electrostatic interaction, or a strand-exchange. In some embodiments, the one or more immunoglobulin chain constant regions (e.g., Fc regions) comprise an amino acid substitution at a position chosen from one or more of 347, 349, 350, 351, 366, 368, 370, 392, 394, 395, 397, 398, 399, 405, 407, or 409, e.g., of the Fc region of human IgG1. In some embodiments, the one or more immunoglobulin chain constant regions (e.g., Fc regions) comprise an amino acid substitution chosen from: T366S, L368A, or Y407V (e.g., corresponding to a cavity or hole), or T366W (e.g., corresponding to a protuberance or knob), or a combination thereof.

In some embodiments, the multifunctional molecule further comprises a linker, e.g., a linker between one or more of: the antigen binding domain and the immune cell engager, the antigen binding domain and the cytokine molecule, the antigen binding domain and the stromal modifying moiety, the immune cell engager and the cytokine molecule, the immune cell engager and the stromal modifying moiety, the cytokine molecule and the stromal modifying moiety, the antigen binding domain and the dimerization module, the immune cell engager and the dimerization module, the cytokine molecule and the dimerization module, or the stromal modifying moiety and the dimerization module. In some embodiments, the linker is chosen from: a cleavable linker, a non-cleavable linker, a peptide linker, a flexible linker, a rigid linker, a helical linker, or a non-helical linker. In some embodiments, the linker is a peptide linker. In some embodiments, the peptide linker comprises Gly and Ser. In some embodiments, the peptide linker comprises an amino acid sequence chosen from SEQ ID NOs: 6214-6217 or 6220-6221 and 77-78.

In one aspect, the invention provides a multifunctional molecule, comprising:
(i) an antigen binding domain that binds to a calreticulin protein (e.g., a wild type calreticulin protein or a calreticulin mutant protein), e.g., wherein the calreticulin mutant protein comprises the amino acid sequence of SEQ ID NO: 6286, and
(ii) a moiety that binds to CD3, e.g., an antibody molecule that binds to CD3.

In some embodiments, the multifunctional molecule comprises: a first polypeptide comprising, e.g., from N-terminus to C-terminus, a first VL and a first CL, a second polypeptide comprising, e.g., from N-terminus to C-terminus, a first VH, a first CH1, a first dimerization domain (e.g., a first Fc), and a first moiety that binds to CD3 (e.g., a first scFv that binds to CD3),
a third polypeptide comprising, e.g., from N-terminus to C-terminus, a second VH, a second CH1, a second dimerization domain (e.g., a second Fc), and optionally a second moiety that binds to CD3 (e.g., a second scFv that binds to CD3),
a fourth polypeptide comprising, e.g., from N-terminus to C-terminus, a second VL and a second CL, wherein:
the first VL and the first VH form a first antigen binding domain that binds to a first calreticulin protein (e.g., a wild-type calreticulin protein or a calreticulin mutant protein), and the second VL and the second VH form a second antigen binding domain that binds to a second calreticulin protein (e.g., a wild-type calreticulin protein or a calreticulin mutant protein), wherein the first and second calreticulin proteins comprise the amino acid sequence of SEQ ID NO: 6286, optionally wherein the first and second calreticulin proteins are each independently chosen from: a molecule comprising the amino acid sequence of SEQ ID NO: 6313, or a molecule comprising the amino acid sequence of SEQ ID NO: 6314.

In some embodiments, the multifunctional molecule comprises the configuration of FIG. 2A or 2B.

In one aspect, the invention provides a multifunctional molecule, comprising:
(i) an antigen binding domain that binds to a calreticulin protein (e.g., a wild type calreticulin protein or a calreticulin mutant protein), e.g., wherein the calreticulin mutant protein comprises the amino acid sequence of SEQ ID NO: 6286, and
(ii) a moiety that binds to TCR (e.g., TCRβ), e.g., an antibody molecule that binds to TCR (e.g., TCRβ).

In some embodiments, the multifunctional molecule comprises: a first polypeptide comprising, e.g., from N-terminus to C-terminus, a first VL and a first CL, a second polypeptide comprising, e.g., from N-terminus to C-terminus, a first VH, a first CH1, a first dimerization domain (e.g., a first Fc), and a first moiety that binds to TCR (e.g., TCRβ) (e.g., a first scFv that binds to TCR (e.g., TCRβ)),
a third polypeptide comprising, e.g., from N-terminus to C-terminus, a second VH, a second CH1, a second dimerization domain (e.g., a second Fc), and optionally a second moiety that binds to TCR (e.g., TCRβ) (e.g., a second scFv that binds to TCR (e.g., TCRβ)), a fourth polypeptide comprising, e.g., from N-terminus to C-terminus, a second VL and a second CL, wherein:
the first VL and the first VH form a first antigen binding domain that binds to a first calreticulin protein (e.g., a wild-type calreticulin protein or a calreticulin mutant protein), and the second VL and the second VH form a second antigen binding domain that binds to a second calreticulin protein (e.g., a wild-type calreticulin protein or a calreticulin mutant protein), wherein the first and second calreticulin proteins comprise the amino acid sequence of SEQ ID NO: 6286, optionally wherein the first and second calreticulin proteins are each independently chosen from: a molecule comprising the amino acid sequence of SEQ ID NO: 6313, or a molecule comprising the amino acid sequence of SEQ ID NO: 6314.

In some embodiments, the multifunctional molecule comprises the configuration of FIG. 3A or 3B.

In one aspect, the invention provides a multifunctional molecule, comprising:
(i) an antigen binding domain that binds to a calreticulin protein (e.g., a wild-type calreticulin protein or a calreticulin mutant protein), e.g., wherein the calreticulin protein comprises the amino acid sequence of SEQ ID NO: 6286, and
(ii) a moiety that binds to NKp30, e.g., an antibody molecule or ligand that binds to (e.g., activates) NKp30.

In some embodiments, the multifunctional molecule comprises:
a first polypeptide comprising, e.g., from N-terminus to C-terminus, a first VL and a first CL, a second polypeptide comprising, e.g., from N-terminus to C-terminus, a first VH, a first CH1, a first dimerization domain (e.g., a first Fc), and a first moiety that binds to NKp30 (e.g., a first antibody molecule or ligand that binds to NKp30),
a third polypeptide comprising, e.g., from N-terminus to C-terminus, a second VH, a second CH1, a second dimerization domain (e.g., a second Fc), and optionally a second moiety that binds to NKp30 (e.g., a second antibody molecule or ligand that binds to NKp30), a fourth polypeptide comprising, e.g., from N-terminus to C-terminus, a second VL and a second CL, wherein:
the first VL and the first VH form a first antigen binding domain that binds to a first calreticulin protein (e.g., a wild-type calreticulin protein or a calreticulin mutant protein), and the second VL and the second VH form a second antigen binding domain that binds to a second calreticulin protein (e.g., a wild-type calreticulin protein or a calreticulin mutant protein), wherein the first and second calreticulin proteins comprise the amino acid sequence of SEQ ID NO: 6286, optionally wherein the first and second calreticulin proteins are each independently chosen from: a molecule comprising the amino acid sequence of SEQ ID NO: 6313, or a molecule comprising the amino acid sequence of SEQ ID NO: 6314.

In some embodiments, the multifunctional molecule comprises the configuration of FIG. 4A or 4B.

In another aspect, the disclosure provides an isolated nucleic acid molecule encoding any multispecific or multifunctional molecule described herein. In another aspect, the disclosure provides an isolated nucleic acid molecule, which comprises the nucleotide sequence encoding any of the multispecific or multifunctional molecules described herein, or a nucleotide sequence substantially homologous thereto (e.g., at least 80%, 90%, 95%, or 99.9% identical thereto). In another aspect, the disclosure provides a host cell comprising a nucleic acid molecule or a vector described herein.

In another aspect, the disclosure provides a method of making, e.g., producing, a multispecific or multifunctional molecule polypeptide described herein, comprising culturing a host cell described herein, under suitable conditions, e.g., conditions suitable for gene expression and/or homo- or heterodimerization.

In another aspect, the disclosure provides a pharmaceutical composition comprising a multispecific or multifunctional molecule polypeptide described herein and a pharmaceutically acceptable carrier, excipient, or stabilizer.

In another aspect, the disclosure provides a method of treating a cancer, comprising administering to a subject in need thereof a multispecific or multifunctional molecule polypeptide described herein, wherein the multispecific antibody is administered in an amount effective to treat the cancer. In some embodiments, the subject has cancer cells that express the first and/or second calreticulin mutant. In some embodiments, the subject has tumor cells that express the first, second, or third tumor antigen, e.g., the subject has tumor cells that express a tumor antigen chosen from G6B, CD34, CD41, P-selectin, Clec2, cKIT, FLT3, MPL, ITGB3, ITGB2, GP5, GP6, GP9, GP1BA, DSC2, FCGR2A, TNFRSF10A, TNFRSF10B, or TM4SF1. In some embodiments, the subject has the JAK2 V617F mutation. In some embodiments, the subject does not have the JAK2 V617F mutation. In some embodiments, the subject has a MPL mutation. In some embodiments, the subject does not have a MPL mutation. In some embodiments, the cancer is a hematological cancer, optionally wherein the cancer is a myeloproliferative neoplasm, e.g., primary or idiopathic myelofibrosis (MF), essential thrombocytosis (ET), polycythemia vera (PV), or chronic myelogenous leukemia (CML). In some embodiments, the cancer is myelofibrosis. In some embodiments, the cancer is a solid tumor cancer. In some embodiments, the solid tumor cancer is one or more of pancreatic (e.g., pancreatic adenocarcinoma), breast, colorectal, lung (e.g., small or non-small cell lung cancer), skin, ovarian, or liver cancer.

In some embodiments, the cancer cell comprises a myeloproliferative neoplasm cell. In some embodiments, the myeloproliferative neoplasm cell is chosen from a myelofibrosis cell, an essential thrombocythemia cell, a polycythemia vera cell, or a chronic myeloid cancer cell. In some embodiments, the myeloproliferative neoplasm cell is a myelofibrosis cell. In some embodiments, the myeloproliferative neoplasm cell is an essential thrombocythemia cell. In some embodiments, the myeloproliferative neoplasm cell is a polycythemia vera cell. In some embodiments, the myeloproliferative neoplasm cell is a chronic myeloid cancer cell. In some embodiments, the myeloproliferative neoplasm cell comprises a JAK2 mutation (e.g., a JAK2 V617F mutation). In some embodiments, the myeloproliferative neoplasm cell comprises a calreticulin mutation. In some embodiments, the myeloproliferative neoplasm cell comprises a MPL mutation.

In some embodiments, the method further comprises administering a second therapeutic treatment. In some embodiments, second therapeutic treatment comprises a therapeutic agent (e.g., a chemotherapeutic agent, a biologic agent, hormonal therapy), radiation, or surgery. In some embodiments, therapeutic agent is selected from: a chemotherapeutic agent, or a biologic agent.

### Exemplary Embodiment 2

1. A multifunctional molecule comprising:
(i) a first antigen binding domain that binds to a calreticulin protein (e.g., a wild-type or mutant calreticulin protein), e.g., a calreticulin-targeting antigen binding domain disclosed in any one of Table 4, Table 5, Table 6, Table 24, Table 25, Table 16, Table 17, Table 18, or Table 19, and
(ii) a second antigen binding domain that binds to TCRβV, e.g., an anti-TCRβV antigen binding domain disclosed in any one of Table 30, Table 31, Table 32, Table 33, Table 11, Table 12, or Table 13, or a second antigen binding domain that binds to NKp30, e.g., an anti-NKp30 antigen binding domain disclosed in Table 7, Table 8, Table 35, Table 36, Table 9, Table 10, or Table 34.

2. The multifunctional molecule of embodiment 1, wherein the second antigen binding domain binds to TCRβV.

3. The multifunctional molecule of embodiment 2, wherein the second antigen binding domain activates a T cell or the second antigen binding domain does not activate a T cell.

4. The multifunctional molecule of embodiment 2 or 3, wherein the second antigen binding domain binds to TCRβ V12 or TCRβ V6 (e.g., comprising the amino acid sequence of SEQ ID NO: 1044).

5. The multifunctional molecule of any of embodiments 2-4, wherein the second antigen binding domain comprises one or more amino acid sequences as listed in Table 30, Table 31, Table 32, Table 33, Table 11, Table 12, or Table 13.

6. The multifunctional molecule of any of embodiments 2-5, wherein the second antigen binding domain comprises:
(a) a heavy chain variable region (VH) and/or a light chain variable region (VL), wherein:
(i) the VH comprises a heavy chain complementarity determining region 1 (VHCDR1) having an amino acid sequence of a VHCDR1 in Table 30, Table 31, Table 33, Table 11, Table 12, or Table 13 (or a sequence with no more than 1, 2, 3, or 4 mutations, e.g., substitutions, additions, or deletions), a VHCDR2 having an amino acid sequence of a VHCDR2 in Table 30, Table 31, Table 33, Table 11, Table 12, or Table 13 (or a sequence with no more than 1, 2, 3, or 4 mutations, e.g., substitutions, additions, or deletions), and/or a VHCDR3 having an amino acid sequence of a VHCDR3 in Table 30, Table 31, Table 33, Table 11, Table 12, or Table 13 (or a sequence with no more than 1, 2, 3, or 4 mutations, e.g., substitutions, additions, or deletions),
(ii) the VL comprises a light chain complementarity determining region 1 (VLCDR1) having an amino acid sequence of a VLCDR1 in Table 30, Table 31, Table 33, Table 11, Table 12, or Table 13 (or a sequence with no more than 1, 2, 3, or 4 mutations, e.g., substitutions, additions, or deletions), a VLCDR2 having an amino acid sequence of a VLCDR2 in Table 30, Table 31, Table 33, Table 11, Table 12, or Table 13 (or a sequence with no more than 1, 2, 3, or 4 mutations, e.g., substitutions, additions, or deletions), and/or a VLCDR3 having an amino acid sequence of a VLCDR3 in Table 30, Table 31, Table 33, Table 11, Table 12, or Table 13 (or a sequence with no more than 1, 2, 3, or 4 mutations, e.g., substitutions, additions, or deletions);
(b) a heavy chain variable region (VH) and/or a light chain variable region (VL), wherein:
(i) the VH comprises a heavy chain complementarity determining region 1 (VHCDR1) amino acid sequence of SEQ ID NO: 3 (or a sequence with no more than 1, 2, 3, or 4 mutations, e.g., substitutions, additions, or deletions), a VHCDR2 amino acid sequence of SEQ ID NO: 4 (or a sequence with no more than 1, 2, 3, or 4 mutations, e.g., substitutions, additions, or deletions), and/or a VHCDR3 amino acid sequence of SEQ ID NO: 5 (or a sequence with no more than 1, 2, 3, or 4 mutations, e.g., substitutions, additions, or deletions), and/or
(ii) the VL comprises a light chain complementarity determining region 1 (VHCDR1) amino acid sequence of SEQ ID NO: 6 (or a sequence with no more than 1, 2, 3, or 4 mutations, e.g., substitutions, additions, or deletions), a VHCDR2 amino acid sequence of SEQ ID NO: 7 (or a sequence with no more than 1, 2, 3, or 4 mutations, e.g., substitutions, additions, or deletions), and/or a VHCDR3 amino acid sequence of SEQ ID NO: 8 (or a sequence with no more than 1, 2, 3, or 4 mutations, e.g., substitutions, additions, or deletions);
(c) a heavy chain variable region (VH) and/or a light chain variable region (VL), wherein:
(i) the VH comprises a heavy chain complementarity determining region 1 (VHCDR1) amino acid sequence of SEQ ID NO: 45 (or a sequence with no more than 1, 2, 3, or 4 mutations, e.g., substitutions, additions, or deletions), a VHCDR2 amino acid sequence of SEQ ID NO: 46 (or a sequence with no more than 1, 2, 3, or 4 mutations, e.g., substitutions, additions, or deletions), and/or a VHCDR3 amino acid sequence of SEQ ID NO: 47 (or a sequence with no more than 1, 2, 3, or 4 mutations, e.g., substitutions, additions, or deletions), and/or
(ii) the VL comprises a light chain complementarity determining region 1 (VHCDR1) amino acid sequence of SEQ ID NO: 51 (or a sequence with no more than 1, 2, 3, or 4 mutations, e.g., substitutions, additions, or deletions), a VHCDR2 amino acid sequence of SEQ ID NO: 52 (or a sequence with no more than 1, 2, 3, or 4 mutations, e.g., substitutions, additions, or deletions), and/or a VHCDR3 amino acid sequence of SEQ ID NO: 53 (or a sequence with no more than 1, 2, 3, or 4 mutations, e.g., substitutions, additions, or deletions); and/or
(d) a heavy chain variable region (VH) and/or a light chain variable region (VL), wherein:
(i) the VH comprises a heavy chain complementarity determining region 1 (VHCDR1) amino acid sequence of SEQ ID NO: 48 (or a sequence with no more than 1, 2, 3, or 4 mutations, e.g., substitutions, additions, or deletions), a VHCDR2 amino acid sequence of SEQ ID NO: 49 (or a sequence with no more than 1, 2, 3, or 4 mutations, e.g., substitutions, additions, or deletions), and/or a VHCDR3 amino acid sequence of SEQ ID NO: 50 (or a sequence with no more than 1, 2, 3, or 4 mutations, e.g., substitutions, additions, or deletions), and/or
(ii) the VL comprises a light chain complementarity determining region 1 (VHCDR1) amino acid sequence of SEQ ID NO: 54 (or a sequence with no more than 1, 2, 3, or 4 mutations, e.g., substitutions, additions, or deletions), a VHCDR2 amino acid sequence of SEQ ID NO: 55 (or a sequence with no more than 1, 2, 3, or 4 mutations, e.g., substitutions, additions, or deletions), and/or a VHCDR3 amino acid sequence of SEQ ID NO: 56 (or a sequence with no more than 1, 2, 3, or 4 mutations, e.g., substitutions, additions, or deletions).

7. The multifunctional molecule of any of embodiments 2-5, wherein the second antigen binding domain comprises:
(a) a heavy chain variable region (VH) and/or a light chain variable region (VL), wherein:
(i) the VH comprises the amino acid sequence of a VH in Table 30, Table 31, Table 33, Table 11, Table 12, or Table 13 (or an amino acid sequence having at least about 77%, 80%, 85%, 90%, 95%, or 99% sequence identity thereto), and/or
(ii) the VL comprises the amino acid sequence of a VL in Table 30, Table 31, Table 33, Table 11, Table 12, or Table 13 (or an amino acid sequence having at least about 77%, 80%, 85%, 90%, 95%, or 99% sequence identity thereto)
(iii) the VH comprises the amino acid sequence of SEQ ID NO: 9 (or an amino acid sequence having at least about 77%, 80%, 85%, 90%, 95%, or 99% sequence identity thereto), and/or
(iv) the VL comprises the amino acid sequence of SEQ ID NO: 10 (or an amino acid sequence having at least about 77%, 80%, 85%, 90%, 95%, or 99% sequence identity thereto);
(b) a heavy chain variable region (VH) and/or a light chain variable region (VL), wherein:
(i) the VH comprises the amino acid sequence of SEQ ID NO: 9 (or an amino acid sequence having at least about 77%, 80%, 85%, 90%, 95%, or 99% sequence identity thereto), and/or
(ii) the VL comprises the amino acid sequence of SEQ ID NO: 11 (or an amino acid sequence having at least about 77%, 80%, 85%, 90%, 95%, or 99% sequence identity thereto); and/or
(c) a heavy chain variable region (VH) and/or a light chain variable region (VL), wherein:
(i) the VH comprises the amino acid sequence of SEQ ID NO: 1312 (or an amino acid sequence having at least about 77%, 80%, 85%, 90%, 95%, or 99% sequence identity thereto), and/or
(ii) the VL comprises the amino acid sequence of SEQ ID NO: 1314 (or an amino acid sequence having at least about 77%, 80%, 85%, 90%, 95%, or 99% sequence identity thereto).

8. The multifunctional molecule of any of embodiments 2-5, wherein the second antigen binding domain comprises:
(a) a heavy chain variable region (VH) and/or a light chain variable region (VL), wherein:
(i) the VH comprises a heavy chain complementarity determining region 1 (VHCDR1) amino acid sequence of SEQ ID NO: 17 (or a sequence with no more than 1, 2, 3, or 4 mutations, e.g., substitutions, additions, or deletions), a VHCDR2 amino acid sequence of SEQ ID NO: 18 (or a sequence with no more than 1, 2, 3, or 4 mutations, e.g., substitutions, additions, or deletions), and/or a VHCDR3 amino acid sequence of SEQ ID NO: 19 (or a sequence with no more than 1, 2, 3, or 4 mutations, e.g., substitutions, additions, or deletions), and/or
(ii) the VL comprises a light chain complementarity determining region 1 (VHCDR1) amino acid sequence of SEQ ID NO: 20 (or a sequence with no more than 1, 2, 3, or 4 mutations, e.g., substitutions, additions, or deletions), a VHCDR2 amino acid sequence of SEQ ID NO: 21 (or a sequence with no more than 1, 2, 3, or 4 mutations, e.g., substitutions, additions, or deletions), and/or a VHCDR3 amino acid sequence of SEQ ID NO: 22 (or a sequence with no more than 1, 2, 3, or 4 mutations, e.g., substitutions, additions, or deletions);
(b) a heavy chain variable region (VH) and/or a light chain variable region (VL), wherein:
(i) the VH comprises a heavy chain complementarity determining region 1 (VHCDR1) amino acid sequence of SEQ ID NO: 57 (or a sequence with no more than 1, 2, 3, or 4 mutations, e.g., substitutions, additions, or deletions), a VHCDR2 amino acid sequence of SEQ ID NO: 58 (or a sequence with no more than 1, 2, 3, or 4 mutations, e.g., substitutions, additions, or deletions), and/or a VHCDR3 amino acid sequence of SEQ ID NO: 59 (or a sequence with no more than 1, 2, 3, or 4 mutations, e.g., substitutions, additions, or deletions), and/or
(ii) the VL comprises a light chain complementarity determining region 1 (VHCDR1) amino acid sequence of SEQ ID NO: 63 (or a sequence with no more than 1, 2, 3, or 4 mutations, e.g., substitutions, additions, or deletions), a VHCDR2 amino acid sequence of SEQ ID NO: 64 (or a sequence with no more than 1, 2, 3, or 4 mutations, e.g., substitutions, additions, or deletions), and/or a VHCDR3 amino acid sequence of SEQ ID NO: 65 (or a sequence with no more than 1, 2, 3, or 4 mutations, e.g., substitutions, additions, or deletions); and/or
(c) a heavy chain variable region (VH) and/or a light chain variable region (VL), wherein:
(i) the VH comprises a heavy chain complementarity determining region 1 (VHCDR1) amino acid sequence of SEQ ID NO: 60 (or a sequence with no more than 1, 2, 3, or 4 mutations, e.g., substitutions, additions, or deletions), a VHCDR2 amino acid sequence of SEQ ID NO: 61 (or a sequence with no more than 1, 2, 3, or 4 mutations, e.g., substitutions, additions, or deletions), and/or a VHCDR3 amino acid sequence of SEQ ID NO: 62 (or a sequence with no more than 1, 2, 3, or 4 mutations, e.g., substitutions, additions, or deletions), and/or
(ii) the VL comprises a light chain complementarity determining region 1 (VHCDR1) amino acid sequence of SEQ ID NO: 66 (or a sequence with no more than 1, 2, 3, or 4 mutations, e.g., substitutions, additions, or deletions), a VHCDR2 amino acid sequence of SEQ ID NO: 67 (or a sequence with no more than 1, 2, 3, or 4 mutations, e.g., substitutions, additions, or deletions), and/or a VHCDR3 amino acid sequence of SEQ ID NO: 68 (or a sequence with no more than 1, 2, 3, or 4 mutations, e.g., substitutions, additions, or deletions).

9. The multifunctional molecule of any of embodiments 2-5, wherein the second antigen binding domain comprises:
(a) a heavy chain variable region (VH) and/or a light chain variable region (VL), wherein:
(i) the VH comprises the amino acid sequence of SEQ ID NO: 15 (or an amino acid sequence having at least about 77%, 80%, 85%, 90%, 95%, or 99% sequence identity thereto), and/or
(ii) the VL comprises the amino acid sequence of SEQ ID NO: 16 (or an amino acid sequence having at least about 77%, 80%, 85%, 90%, 95%, or 99% sequence identity thereto); and/or
(b) a heavy chain variable region (VH) and/or a light chain variable region (VL), wherein:
(i) the VH comprises: the amino acid sequence of SEQ ID NO: 23 (or an amino acid sequence having at least about 77%, 80%, 85%, 90%, 95%, or 99% sequence identity thereto), the amino acid sequence of SEQ ID NO: 24 (or an amino acid sequence having at least about 77%, 80%, 85%, 90%, 95%, or 99% sequence identity thereto), or the amino acid sequence of SEQ ID NO: 25 (or an amino acid sequence having at least about 77%, 80%, 85%, 90%, 95%, or 99% sequence identity thereto); and/or
(ii) the VL comprises:
the amino acid sequence of SEQ ID NO: 26 (or an amino acid sequence having at least about 77%, 80%, 85%, 90%, 95%, or 99% sequence identity thereto),
the amino acid sequence of SEQ ID NO: 27 (or an amino acid sequence having at least about 77%, 80%, 85%, 90%, 95%, or 99% sequence identity thereto),
the amino acid sequence of SEQ ID NO: 28 (or an amino acid sequence having at least about 77%, 80%, 85%, 90%, 95%, or 99% sequence identity thereto),
the amino acid sequence of SEQ ID NO: 29 (or an amino acid sequence having at least about 77%, 80%, 85%, 90%, 95%, or 99% sequence identity thereto), or
the amino acid sequence of SEQ ID NO: 30 (or an amino acid sequence having at least about 77%, 80%, 85%, 90%, 95%, or 99% sequence identity thereto).

10. The multifunctional molecule of any of embodiments 2-9, comprising:
a first polypeptide comprising, e.g., from N-terminus to C-terminus, a first VL and a first CL, a second polypeptide comprising, e.g., from N-terminus to C-terminus, a first VH, a first CH1, a first dimerization domain (e.g., a first Fc), and a first moiety that binds to TCR (e.g., TCRVβ) (e.g., a first scFv that binds to TCR (e.g., TCRVβ)),
a third polypeptide comprising, e.g., from N-terminus to C-terminus, a second VH, a second CH1, a second dimerization domain (e.g., a second Fc), and optionally a second moiety that binds to TCR (e.g., TCRVβ) (e.g., a second scFv that binds to TCR (e.g., TCRVβ)),
a fourth polypeptide comprising, e.g., from N-terminus to C-terminus, a second VL and a second CL, wherein:
the first VL and the first VH form a first antigen binding domain that binds to a first calreticulin protein, and the second VL and the second VH form a third antigen binding domain that binds to a second calreticulin protein,
optionally wherein the first and second calreticulin proteins comprise the amino acid sequence of SEQ ID NO: 6285, 1001, or 6286,
optionally wherein the first and second calreticulin mutant proteins are each independently chosen from: a molecule comprising the amino acid sequence of SEQ ID NO: 6313, or a molecule comprising the amino acid sequence of SEQ ID NO: 6314, optionally wherein the multifunctional molecule comprises the configuration of FIG. 3A or 3B.

11. The multifunctional molecule of embodiment 1, wherein the second antigen binding domain binds to NKp30.

12. The multifunctional molecule of embodiment 11, wherein the second antigen binding domain is chosen from an antibody molecule, e.g., an antigen binding domain, or ligand that binds to (e.g., activates) NKp30, e.g., the second antigen binding domain is an antibody molecule or ligand that binds to (e.g., activates) NKp30.

13. The multifunctional molecule of embodiment 11 or 12, wherein the second antigen binding domain comprises:
(i) a heavy chain variable region (VH) comprising a heavy chain complementarity determining region 1 (VHCDR1) having an amino acid sequence of a VHCDR1 of Table 7, Table 35, Table 9, Table 10, or Table 34 (or a sequence with no more than 1, 2, 3, or 4 mutations, e.g., substitutions, additions, or deletions), a VHCDR2 having an amino acid sequence of a VHCDR2 of Table 7, Table 35, Table 9, Table 10, or Table 34 (or a sequence with no more than 1, 2, 3, or 4 mutations, e.g., substitutions, additions, or deletions), and/or a VHCDR3 having an amino acid sequence of a VHCDR3 of Table 7, Table 35, Table 9, Table 10, or Table 34 (or a sequence with no more than 1, 2, 3, or 4 mutations, e.g., substitutions, additions, or deletions), and/or
(ii) a light chain variable region (VL) comprising a light chain complementarity determining region 1 (VLCDR1) having an amino acid sequence of a VLCDR1 of Table 8, Table 36, Table 9, Table 10, or Table 34 (or a sequence with no more than 1, 2, 3, or 4 mutations, e.g., substitutions, additions, or deletions), a VLCDR2 having an amino acid sequence of a VLCDR2 of Table 8, Table 36, Table 9, Table 10, or Table 34 (or a sequence with no more than 1, 2, 3, or 4 mutations, e.g., substitutions, additions, or deletions), and/or a VLCDR3 having an amino acid sequence of a VLCDR3 of Table 8, Table 36, Table 9, Table 10, or Table 34 (or a sequence with no more than 1, 2, 3, or 4 mutations, e.g., substitutions, additions, or deletions).

14. The multifunctional molecule of embodiment 13, wherein the second antigen binding domain comprises:
(i) a heavy chain variable region (VH) comprising a heavy chain complementarity determining region 1 (VHCDR1) amino acid sequence of SEQ ID NO: 7313 (or a sequence with no more than 1, 2, 3, or 4 mutations, e.g., substitutions, additions, or deletions), a VHCDR2 amino acid sequence of SEQ ID NO: 6001 (or a sequence with no more than 1, 2, 3, or 4 mutations, e.g., substitutions, additions, or deletions, and/or a VHCDR3 amino acid sequence of SEQ ID NO: 7315 (or a sequence with no more than 1, 2, 3, or 4 mutations, e.g., substitutions, additions, or deletions; and/or
(ii) a light chain variable region (VL) comprising a light chain complementarity determining region 1 (VLCDR1) amino acid sequence of SEQ ID NO: 7326 (or a sequence with no more than 1, 2, 3, or 4 mutations, e.g., substitutions, additions, or deletions), a VLCDR2 amino acid sequence of SEQ ID NO: 7327 (or a sequence with no more than 1, 2, 3, or 4 mutations, e.g., substitutions, additions, or deletions), and/or a VLCDR3 amino acid sequence of SEQ ID NO: 7329 (or a sequence with no more than 1, 2, 3, or 4 mutations, e.g., substitutions, additions, or deletions).

15. The multifunctional molecule of embodiment 13 or 14, wherein the second antigen binding domain comprises:
(i) a VH comprising the amino acid sequence of any of SEQ ID NOs: 7298 or 7300-7304 (or an amino acid sequence having at least about 75%, 80%, 85%, 90%, 95%, or 99% sequence identity to any of SEQ ID NOs: 7298 or 7300-7304); and/or
(ii) a VL comprising the amino acid sequence of any of SEQ ID NOs: 7299 or 7305-7309 (or an amino acid sequence having at least about 93%, 95%, or 99% sequence identity to any of SEQ ID NOs: 7299 or 7305-7309).

16. The multifunctional molecule of any of embodiments 13-15, wherein the second antigen binding domain comprises:
(i) a VH comprising the amino acid sequence of SEQ ID NO: 7302 (or an amino acid sequence having at least about 75%, 80%, 85%, 90%, 95%, or 99% sequence identity to 7302), and a VL comprising the amino acid sequence of SEQ ID NO: 7305 (or an amino acid sequence having at least about 75%, 80%, 85%, 90%, 95%, or 99% sequence identity to 7305); or
(ii) a VH comprising the amino acid sequence of SEQ ID NO: 7302 (or an amino acid sequence having at least about 75%, 80%, 85%, 90%, 95%, or 99% sequence identity to 7302), and a VL comprising the amino acid sequence of SEQ ID NO: 7309 (or an amino acid sequence having at least about 75%, 80%, 85%, 90%, 95%, or 99% sequence identity to 7309).

17. The multifunctional molecule of any of embodiments 13-16, wherein the second antigen binding domain comprises:
(i) an amino acid sequence of SEQ ID NO: 7310 (or an amino acid sequence having at least about 75%, 80%, 85%, 90%, 95%, or 99% sequence identity to 7310); or
(ii) an amino acid sequence of SEQ ID NO: 7311 (or an amino acid sequence having at least about 75%, 80%, 85%, 90%, 95%, or 99% sequence identity to 7311).

18. The multifunctional molecule of embodiment 11 or 12, wherein the second antigen binding domain comprises:
(i) a heavy chain variable region (VH) comprising a heavy chain complementarity determining region 1 (VHCDR1) amino acid sequence of SEQ ID NO: 6000, a VHCDR2 amino acid sequence of SEQ ID NO: 6001, and/or a VHCDR3 amino acid sequence of SEQ ID NO: 6002, and
(ii) a light chain variable region (VL) comprising a light chain complementarity determining region 1 (VLCDR1) amino acid sequence of SEQ ID NO: 6063, a VLCDR2 amino acid sequence of SEQ ID NO: 6064, and/or a VLCDR3 amino acid sequence of SEQ ID NO: 7293.

19. The multifunctional molecule of any of embodiments 11, 12, or 18, wherein the second antigen binding domain comprises:
(1) a heavy chain variable region (VH) comprising a heavy chain framework region 1 (VHFWR1) having an amino acid sequence of a VHFWR1 of Table 7, Table 35, Table 9, Table 10, or Table 34 (or a sequence with no more than 1, 2, 3, 4, 5, or 6 mutations, e.g., substitutions, additions, or deletions, therefrom), a VHFWR2 having an amino acid sequence of a VHFWR2 of Table 7, Table 35, Table 9, Table 10, or Table 34 (or a sequence with no more than 1, 2, 3, 4, 5, or 6 mutations, e.g., substitutions, additions, or deletions, therefrom), a VHFWR3 having an amino acid sequence of a VHFWR3 of Table 7, Table 35, Table 9, Table 10, or Table 34 (or a sequence with no more than 1, 2, 3, 4, 5, or 6 mutations, e.g., substitutions, additions, or deletions, therefrom), or a VHFWR4 having an amino acid sequence of a VHFWR4 of Table 7, Table 35, Table 9, Table 10, or Table 34 (or a sequence with no more than 1, 2, 3, 4, 5, or 6 mutations, e.g., substitutions, additions, or deletions, therefrom), and/or
(2) a light chain variable region (VL) comprising a light chain framework region 1 (VLFWR1) having an amino acid sequence of a VLFWR1 of Table 8, Table 36, Table 9, Table 10, or Table 34 (or a sequence with no more than 1, 2, 3, 4, 5, or 6 mutations, e.g., substitutions, additions, or deletions, therefrom), a VLFWR2 having an amino acid sequence of a VLFWR2 of Table 8, Table 36, Table 9, Table 10, or Table 34 (or a sequence with no more than 1, 2, 3, 4, 5, or 6 mutations, e.g., substitutions, additions, or deletions, therefrom), a VLFWR3 having an amino acid sequence of a VLFWR3 of Table 8, Table 36, Table 9, Table 10, or Table 34 (or a sequence with no more than 1, 2, 3, 4, 5, or 6 mutations, e.g., substitutions, additions, or deletions, therefrom), or a VLFWR4 having an amino acid sequence of a VLFWR4 of Table 8, Table 36, Table 9, Table 10, or Table 34 (or a sequence with no more than 1, 2, 3, 4, 5, or 6 mutations, e.g., substitutions, additions, or deletions, therefrom).

20. The multifunctional molecule of embodiment 19, wherein the second antigen binding domain comprises:
(1) a heavy chain variable region (VH) comprising a heavy chain framework region 1 (VHFWR1) amino acid sequence of SEQ ID NO: 6003, a VHFWR2 amino acid sequence of SEQ ID NO: 6004, a VHFWR3 amino acid sequence of SEQ ID NO: 6005, or a VHFWR4 amino acid sequence of SEQ ID NO: 6006, and
(3) a light chain variable region (VL) comprising a light chain framework region 1 (VLFWR1) amino acid sequence of SEQ ID NO: 6066, a VLFWR2 amino acid sequence of SEQ ID NO: 6067, a VLFWR3 amino acid sequence of SEQ ID NO: 7292, or a VLFWR4 amino acid sequence of SEQ ID NO: 6069.

21. The multifunctional molecule of any one of embodiments 11, 12, or 18-20, wherein the second antigen binding domain comprises:
(i) a VH comprising the amino acid sequence of a VH of Table 7, Table 35, Table 9, Table 10, or Table 34 (or an amino acid sequence having at least about 75%, 80%, 85%, 90%, 95%, or 99% sequence identity thereto), and/or
(ii) a VL comprising the amino acid sequence of a VL of Table 8, Table 36, Table 9, Table 10, or Table 34 (or an amino acid sequence having at least about 93%, 95%, or 99% sequence identity thereto).

22. The multifunctional molecule of either of embodiments 11, 12, or 18-21, wherein the second antigen binding domain comprises a heavy chain comprising the amino acid sequence of a heavy chain of Table 10 (or an amino acid sequence having at least about 75%, 80%, 85%, 90%, 95%, or 99% sequence identity thereto).

23. The multifunctional molecule of either of embodiments 11, 12, or 18-22, wherein the second antigen binding domain comprises a light chain comprising the amino acid sequence of a light chain of Table 10 (or an amino acid sequence having at least about 75%, 80%, 85%, 90%, 95%, or 99% sequence identity thereto).

24. The multifunctional molecule of either of embodiments 11, 12, or 18-23, wherein the second antigen binding domain comprises a heavy chain comprising the amino acid sequence of a heavy chain of Table 10 (or an amino acid sequence having at least about 75%, 80%, 85%, 90%, 95%, or 99% sequence identity thereto), and a light chain comprising the amino acid sequence of a light chain of Table 10 (or an amino acid sequence having at least about 75%, 80%, 85%, 90%, 95%, or 99% sequence identity thereto).

25. The multifunctional molecule of any of embodiments 11-24, comprising:
a first polypeptide comprising, e.g., from N-terminus to C-terminus, a first VL and a first CL, a second polypeptide comprising, e.g., from N-terminus to C-terminus, a first VH, a first CH1, a first dimerization domain (e.g., a first Fc), and a first moiety that binds to NKp30 (e.g., a first antibody molecule or ligand that binds to NKp30),
a third polypeptide comprising, e.g., from N-terminus to C-terminus, a second VH, a second CH1, a second dimerization domain (e.g., a second Fc), and optionally a second moiety that binds to NKp30 (e.g., a second antibody molecule or ligand that binds to NKp30),
a fourth polypeptide comprising, e.g., from N-terminus to C-terminus, a second VL and a second CL, wherein:
the first VL and the first VH form a first antigen binding domain that binds to a first calreticulin protein, and the second VL and the second VH from a third antigen binding domain that binds to a second calreticulin protein,
optionally wherein the first and second calreticulin proteins comprise the amino acid sequence of SEQ ID NO: 6285, 1001, or 6286,
optionally wherein the first and second calreticulin mutant proteins are each independently chosen from: a molecule comprising the amino acid sequence of SEQ ID NO: 6313, or a molecule comprising the amino acid sequence of SEQ ID NO: 6314, optionally wherein the multifunctional molecule comprises the configuration of FIG. 3A or 3B.

26. The multifunctional molecule of any of the preceding embodiments, wherein the calreticulin protein comprises an amino acid sequence chosen from SEQ ID NOs: 6285-6312 or 1001, optionally wherein the calreticulin protein comprises an amino acid sequence chosen from SEQ ID NOs: 6313-6346 or 1002-1003.

27. The multifunctional molecule of any of the preceding embodiments, wherein the calreticulin protein comprises the amino acid sequence of SEQ ID NO: 6285 or 1001.

28. The multifunctional molecule of any of the preceding embodiments, wherein the calreticulin protein comprises the amino acid sequence of SEQ ID NO: 6286.

29. The multifunctional molecule of any of the preceding embodiments, wherein the first antigen binding domain binds to an epitope located within the C-terminus of the calreticulin protein, optionally wherein the first antigen binding domain binds to an epitope located within the amino acid sequence of SEQ ID NO: 6285, 1001, or 6286.

30. The multifunctional molecule of any of the preceding embodiments, further comprising a third antigen binding domain that binds to a second calreticulin protein, e.g., wherein the second calreticulin mutant protein comprises the amino acid sequence of SEQ ID NO: 6285, 1001, or 6286, optionally wherein:
(i) the third antigen binding domain is different from the first antigen binding domain, or
(ii) the third antigen binding domain is the same as the first antigen binding domain.

31. The multifunctional molecule of embodiment 30, wherein the second calreticulin molecule is the same as the calreticulin molecule bound by the first antigen binding domain.

32. The multifunctional molecule of embodiment 30, wherein the second calreticulin molecule is different from the calreticulin molecule bound by the first antigen binding domain.

33. The multifunctional molecule of any of embodiments 30-32, wherein the second calreticulin protein comprises an amino acid sequence chosen from SEQ ID NOs: 6285-6312 or 1001, optionally wherein the second calreticulin protein comprises an amino acid sequence chosen from SEQ ID NOs: 6313-6346 or 1002-1003.

34. The multifunctional molecule of embodiment 33, wherein the calreticulin protein bound by the first antigen binding domain comprises the amino acid sequence of SEQ ID NO: 6285 or 1001, and the second calreticulin protein comprises the amino acid sequence of SEQ ID NO: 6286.

35. The multifunctional molecule of any of embodiments 30-34, wherein the third antigen binding domain binds to an epitope located within the C-terminus of the second calreticulin protein, optionally wherein the third antigen binding domain binds to an epitope located within the amino acid sequence of SEQ ID NO: 6285, 1001, or 6286.

36. The multifunctional molecule of any of the preceding embodiments, wherein the first antigen binding domain comprises:
(i) a heavy chain variable region (VH) comprising a heavy chain complementarity determining region 1 (VHCDR1) having an amino acid sequence of a VHCDR1 in Table 4, Table 24, Table 25, or Table 17 (or a sequence with no more than 1, 2, 3, or 4 mutations, e.g., substitutions, additions, or deletions), a VHCDR2 having an amino acid sequence of a VHCDR2 in Table 4, Table 24, Table 25, or Table 17 (or a sequence with no more than 1, 2, 3, or 4 mutations, e.g., substitutions, additions, or deletions), and/or a VHCDR3 having an amino acid sequence of a VHCDR3 in Table 4, Table 24, Table 25, or Table 17 (or a sequence with no more than 1, 2, 3, or 4 mutations, e.g., substitutions, additions, or deletions);
(ii) a light chain variable region (VL) comprising a light chain complementarity determining region 1 (VHCDR1) having an amino acid sequence of a VLCDR1 in Table 5, Table 24, Table 25, or Table 18 (or a sequence with no more than 1, 2, 3, or 4 mutations, e.g., substitutions, additions, or deletions), a VHCDR2 having an amino acid sequence of a VLCDR2 in Table 5, Table 24, Table 25, or Table 18 (or a sequence with no more than 1, 2, 3, or 4 mutations, e.g., substitutions, additions, or deletions), and/or a VHCDR3 having an amino acid sequence of a VLCDR3 in Table 5, Table 24, Table 25, or Table 18 (or a sequence with no more than 1, 2, 3, or 4 mutations, e.g., substitutions, additions, or deletions);
(iii) a VH comprising the amino acid sequence of a VH in Table 24, Table 25, or Table 16 (or an amino acid sequence having at least about 77%, 80%, 85%, 90%, 95%, or 99% sequence identity thereto);
(iv) a VL comprising the amino acid sequence of a VL in Table 24, Table 25, or Table 16 (or an amino acid sequence having at least about 93%, 95%, or 99% sequence identity thereto);
(v) a VH comprising a heavy chain framework region 1 (VHFWR1) having an amino acid sequence of a VHFWR1 in Table 4 or Table 6 (or a sequence with no more than 1, 2, 3, 4, 5, 6, 7, 8, or 9 mutations, e.g., substitutions, additions, or deletions), a VHFWR2 having an amino acid sequence of a VHFWR2 in Table 4 or Table 6 (or a sequence with no more than 1, 2, 3, 4, 5, 6, 7, 8, or 9 mutations, e.g., substitutions, additions, or deletions), a VHFWR3 having an amino acid sequence of a VHFWR3 in Table 4 or Table 6 (or a sequence with no more than 1, 2, 3, 4, 5, 6, 7, 8, or 9 mutations, e.g., substitutions, additions, or deletions), and/or a VHFWR4 having an amino acid sequence of a VHFWR4 in Table 4 or Table 6 (or a sequence with no more than 1, 2, 3, 4, 5, 6, 7, 8, or 9 mutations, e.g., substitutions, additions, or deletions), and/or
(vi) a VL comprising a light chain framework region 1 (VLFWR1) having an amino acid sequence of a VLFWR1 in Table 5 or Table 6 (or a sequence with no more than 1, 2, 3, 4, 5, 6, 7, 8, or 9 mutations, e.g., substitutions, additions, or deletions), a VLFWR2 having an amino acid sequence of a VLFWR2 in Table 5 or Table 6 (or a sequence with no more than 1, 2, 3, 4, 5, 6, 7, 8, or 9 mutations, e.g., substitutions, additions, or deletions), a VLFWR3 having an amino acid sequence of a VLFWR3 in Table 5 or Table 6 (or a sequence with no more than 1, 2, 3, 4, 5, 6, 7, 8, or 9 mutations, e.g., substitutions, additions, or deletions), and/or a VLFWR4 having an amino acid sequence of a VLFWR4 in Table 5 or Table 6 (or a sequence with no more than 1, 2, 3, 4, 5, 6, 7, 8, or 9 mutations, e.g., substitutions, additions, or deletions).

37. The multifunctional molecule of any of the preceding embodiments, wherein the multifunctional molecule further comprises a tumor-targeting moiety.

38. The multifunctional molecule of embodiment 37, wherein the tumor-targeting moiety binds to a tumor antigen.

39. The multifunctional molecule of embodiment 38, wherein the tumor antigen is selected from G6B, CD34, CD41, P-selectin, Clec2, cKIT, FLT3, MPL, ITGB3, ITGB2, GP5, GP6, GP9, GP1BA, DSC2, FCGR2A, TNFRSF10A, TNFRSF10B, or TM4SF1.

40. The multifunctional molecule of embodiment 37, wherein the tumor-targeting moiety comprises an antibody molecule, e.g., that binds to a tumor antigen selected from G6B, CD34, CD41, P-selectin, Clec2, cKIT, FLT3, MPL, ITGB3, ITGB2, GP5, GP6, GP9, GP1BA, DSC2, FCGR2A, TNFRSF10A, TNFRSF10B, or TM4SF1.

41. The multifunctional molecule of embodiment 40, wherein the tumor-targeting moiety comprises a VH and/or VL sequence, e.g., as listed in Table 38 or Table 20.

42. The multifunctional molecule of any one of the preceding embodiments, wherein the multifunctional molecule preferentially binds to a myeloproliferative neoplasm cell over a non-tumor cell, optionally wherein the binding between the multifunctional molecule and the myeloproliferative neoplasm cell is more than 10, 20, 30, 40, 50-fold greater than the binding between the multifunctional molecule and a non-tumor cell.

43. The multifunctional molecule of embodiment 42, wherein the myeloproliferative neoplasm cell is chosen from a myelofibrosis cell, an essential thrombocythemia cell, a polycythemia vera cell, or a chronic myeloid cancer cell, optionally wherein: the myeloproliferative neoplasm cell does not comprise a JAK2 V617F mutation, or the myeloproliferative neoplasm cell does not comprise a MPL mutation.

44. The multifunctional molecule of any one of the preceding embodiments, further comprising a linker, e.g., a linker between the first antigen binding domain and the second antigen binding domain.

45. The multifunctional molecule of embodiment 44, wherein the linker is chosen from: a cleavable linker, a non-cleavable linker, a peptide linker, a flexible linker, a rigid linker, a helical linker, or a non-helical linker.

46. The multifunctional molecule of embodiment 44 or 45, wherein the linker is a peptide linker.

47. The multifunctional molecule of 46, wherein the peptide linker comprises Gly and Ser.

48. The multifunctional molecule of 46, wherein the peptide linker comprises an amino acid sequence chosen from SEQ ID NOs: 6214-6217 or 6220-6221 and 77-78.

49. A nucleic acid molecule encoding the multifunctional molecule of any of the preceding embodiments.

50. A vector, e.g., an expression vector, comprising the nucleic acid molecule of embodiment 49.

51. A cell comprising the nucleic acid molecule of embodiment 49 or the vector of embodiment 50.

52. A method of making, e.g., producing, the multifunctional molecule of any one of embodiments 1-48, comprising culturing the cell of embodiment 51, under suitable conditions, e.g., conditions suitable for gene expression and/or homo- or heterodimerization.

53. A pharmaceutical composition comprising the multifunctional molecule of any one of embodiments 1-48 and a pharmaceutically acceptable carrier, excipient, or stabilizer.

54. A method of treating a cancer, comprising administering to a subject in need thereof the multifunctional molecule of any one of embodiments 1-48, wherein the multifunctional molecule is administered in an amount effective to treat the cancer.

55. Use of the multifunctional molecule of any one of embodiments 1-48 for the manufacture of a medicament for treating a cancer.

56. The method of embodiment 54 or the use of embodiment 55, wherein the subject has cancer cells that express the first and/or second calreticulin protein.

57. The method of embodiment 54 or 56 or the use of embodiment 55 or 56, wherein the subject has the JAK2 V617F mutation.

58. The method of embodiment 54 or 56 or the use of embodiment 55 or 56, wherein the subject does not have the JAK2 V617F mutation.

59. The method of any one of embodiments 54 or 56-58 or the use of any one of embodiments 55-58, wherein the subject has a MPL mutation.

60. The method of any one of embodiments 54 or 56-58 or the use of any one of embodiments 55-58, wherein the subject does not have a MPL mutation.

61. The method of any one of embodiments 54 or 56-60 or the use of any one of embodiments 55-60, wherein the cancer is a hematological cancer, optionally wherein the cancer is a myeloproliferative neoplasm, e.g., primary or idiopathic myelofibrosis (MF), essential thrombocytosis (ET), polycythemia vera (PV), or chronic myelogenous leukemia (CML), optionally wherein the cancer is myelofibrosis.

62. The method of any one of embodiments 54 or 56-60 or the use of any one of embodiments 55-60, the cancer is a solid tumor cancer.

63. The method of any of embodiments 54 or 56-62 or the use of any one of embodiments 55-62, further comprising administering a second therapeutic treatment.

64. The method of embodiment 63 or the use of embodiment 63, wherein the second therapeutic treatment comprises a therapeutic agent (e.g., a chemotherapeutic agent, a biologic agent, hormonal therapy), radiation, or surgery.

65. The method of embodiment 64 or the use of embodiment 64, wherein the therapeutic agent is selected from: a chemotherapeutic agent, or a biologic agent.

66. A method of detecting calreticulin (e.g., wild-type and/or mutant calreticulin) in a sample or subject, comprising: contacting the sample or subject with an anti-calreticulin (e.g., wild-type and/or mutant calreticulin) antibody molecule described herein; and detecting formation of a complex between the antibody molecule and the sample or subject, thereby detecting calreticulin (e.g., wild-type and/or mutant calreticulin).

67. The method of embodiment 66, wherein calreticulin (e.g., wild-type and/or mutant calreticulin) is detected in vitro or in vivo.

68. The method of embodiment 66 or 67, further comprising contacting a reference sample or subject with the antibody molecule; and detecting formation of a complex between the antibody molecule and the reference sample or subject, wherein a change, e.g., a statistically significant change, in the formation of the complex in the sample or subject, relative to the reference sample or subject is indicative of the presence of calreticulin (e.g., wild-type and/or mutant calreticulin) in the sample or subject.

69. The method of any of embodiments 66-68, further comprising obtaining a sample from a subject.

70. The method of any of embodiment 66-69, wherein sample comprises one or more of plasma, tissue (e.g., cancerous tissue), biopsy, blood (e.g., whole blood), PBMCs, bone marrow, and/or lymphatic tissue, e.g., lymph node.

71. The method of any of embodiments 66-70, wherein the sample has not been frozen and/or fixed.

72. The method of any of embodiments 66-70, wherein the sample has been frozen (e.g., snap frozen) and/or fixed (e.g., formalin-fixed paraffin-embedded (FFPE)).

73. The method of any of embodiments 66-72, wherein the subject has, or is at risk of having, a disease or disorder described herein (e.g., cancer, e.g., myelofibrosis).

74. The method of any of embodiments 66-73, further comprising performing a flow analysis, e.g., using a multi-panel method.

75. The method of any of embodiments 66-74, further comprising assessing T-cell clonality, e.g., to determine the presence and/or level of T cell malignancy.

76. The method of any of embodiments 66-75, further comprising measuring the level of calreticulin+(e.g., wild-type calreticulin+ and/or mutant calreticulin+) cells from the biological sample (e.g., determining if the calreticulin+ cells are depleted, e.g., relative to a reference sample or subject).

77. The method of any of embodiments 66-76, further comprising measuring the intracellular level of calreticulin (e.g., wild-type and/or mutant calreticulin).

78. The method of any of embodiments 66-77, further comprising measuring the membrane level of calreticulin (e.g., wild-type and/or mutant calreticulin).

79. The method of any of embodiments 66-78, further comprising evaluating the subject for a change in prognosis, severity, or presence or absence of a disease or disorder (e.g., cancer, e.g., myelofibrosis), e.g., after treatment (e.g., with an antibody molecule described herein).

80. The method of any of embodiments 66-79, wherein the antibody molecule is detectably labeled.

81. A method of evaluating a subject, comprising: contacting a sample (e.g., a sample described herein) from the subject with an anti-calreticulin (e.g., wild-type and/or mutant calreticulin) antibody molecule described herein; and detecting formation of a complex between the antibody molecule and the sample, thereby evaluating the subject.

82. The method of embodiment 81, wherein the subject has, or is at risk of having, a disease or disorder described herein (e.g., cancer, e.g., myelofibrosis).

83. The method of embodiment 81 or 82, wherein the subject has not been treated with an antibody molecule described herein.

84. The method of embodiment 81 or 82, wherein the subject has been treated with an antibody molecule described herein.

85. A kit comprising an anti-calreticulin (e.g., wild-type and/or mutant calreticulin) antibody molecule described herein and instructions for use in a method of detecting calreticulin (e.g., wild-type and/or mutant calreticulin) in a sample or subject.

1.-135. (canceled)

136. A composition comprising a multifunctional molecule comprising:
(a) a first antigen binding domain that binds to a wild-type or a mutant calreticulin protein, wherein the first antigen binding domain comprises:
(i) a heavy chain variable region (VH) comprising a heavy chain complementarity determining region 1 (VHCDR1), a heavy chain complementarity determining region 2 (VHCDR2), and a heavy chain complementarity determining region 3 (VHCDR3), and
(ii) a light chain variable region (VL) comprising a light chain complementarity determining region 1 (VLCDR1), a light chain complementarity determining region 2 (VLCDR2), and a light chain complementarity determining region 3 (VLCDR3), and
(b) a second antigen binding domain that binds to T cell receptor beta chain variable domain (TCRβV) or NKp30, wherein the second antigen binding domain comprises:
(iii) a heavy chain variable region (VH) comprising a heavy chain complementarity determining region 1 (VHCDR1), a heavy chain complementarity determining region 2 (VHCDR2), and a heavy chain complementarity determining region 3 (VHCDR3), and
(iv) a light chain variable region (VL) comprising a light chain complementarity determining region 1 (VLCDR1), a light chain complementarity determining region 2 (VLCDR2), and a light chain complementarity determining region 3 (VLCDR3),
wherein:
(1) the VHCDR1, VHCDR2, and VHCDR3 of the first antigen binding domain comprise the sequences of:
SEQ ID NO: 7396, SEQ ID NO: 7400, and SEQ ID NO: 7403, respectively; or
SEQ ID NO: 7444, SEQ ID NO: 7400, and SEQ ID NO: 7403, respectively; and
the VLCDR1, VLCDR2, and VLCDR3 of the first antigen binding domain comprise the sequences of:
SEQ ID NO: 7387, SEQ ID NO: 7389, and SEQ ID NO: 7410, respectively;
SEQ ID NO: 7387, SEQ ID NO: 7389, and SEQ ID NO: 7415, respectively;
SEQ ID NO: 7387, SEQ ID NO: 7389, and SEQ ID NO: 7417, respectively; or
SEQ ID NO: 7387, SEQ ID NO: 7389, and SEQ ID NO: 7392, respectively; and/or
(2) the second antigen binding domain binds to NKp30, and the VHCDR1, VHCDR2, and VHCDR3 of the second antigen binding domain comprise the sequences of:
SEQ ID NO: 7498, SEQ ID NO: 6001, SEQ ID NO: 7315, respectively; or
SEQ ID NO: 7498, SEQ ID NO: 7437, SEQ ID NO: 7315, respectively; and
the VLCDR1, VLCDR2, and VLCDR3 of the second antigen binding domain comprise the sequences of:
SEQ ID NO: 7326, SEQ ID NO: 7327, SEQ ID NO: 7416, respectively;
SEQ ID NO: 7494, SEQ ID NO: 7496, SEQ ID NO: 44, respectively;
SEQ ID NO: 7326, SEQ ID NO: 7327, SEQ ID NO: 44, respectively; or
SEQ ID NO: 7326, SEQ ID NO: 7327, SEQ ID NO: 7447, respectively.

137. The composition of claim 136, wherein:
(a) the VHCDR1, VHCDR2, and VHCDR3 of the first antigen binding domain comprise the sequences of:
SEQ ID NO: 6253, SEQ ID NO: 6254, and SEQ ID NO: 6255, respectively;
SEQ ID NO: 6256, SEQ ID NO: 6257, and SEQ ID NO: 6258, respectively;
SEQ ID NO: 6256, SEQ ID NO: 6257, and SEQ ID NO: 6262, respectively;
SEQ ID NO: 6358, SEQ ID NO: 6360, and SEQ ID NO: 227, respectively;
SEQ ID NO: 7396, SEQ ID NO: 7400, and SEQ ID NO: 7403, respectively;
SEQ ID NO: 7444, SEQ ID NO: 7400, and SEQ ID NO: 7403, respectively; or
SEQ ID NO: 6253, SEQ ID NO: 243, and SEQ ID NO: 6255, respectively; and
(b) the VLCDR1, VLCDR2, and VLCDR3 of the first antigen binding domain comprise the sequences of:
SEQ ID NO: 6259, SEQ ID NO: 6260, and SEQ ID NO: 6261, respectively;
SEQ ID NO: 251, SEQ ID NO: 246, and SEQ ID NO: 248, respectively;
SEQ ID NO: 251, SEQ ID NO: 253, and SEQ ID NO: 255, respectively;
SEQ ID NO: 258, SEQ ID NO: 260, and SEQ ID NO: 262, respectively;
SEQ ID NO: 265, SEQ ID NO: 267, and SEQ ID NO: 269, respectively;
SEQ ID NO: 272, SEQ ID NO: 274, and SEQ ID NO: 276, respectively;
SEQ ID NO: 279, SEQ ID NO: 281, and SEQ ID NO: 283, respectively;
SEQ ID NO: 7387, SEQ ID NO: 7389, and SEQ ID NO: 7410, respectively;
SEQ ID NO: 7387, SEQ ID NO: 7389, and SEQ ID NO: 7415, respectively;
SEQ ID NO: 7387, SEQ ID NO: 7389, and SEQ ID NO: 7417, respectively;
SEQ ID NO: 7387, SEQ ID NO: 7389, and SEQ ID NO: 7392, respectively; or
SEQ ID NO: 6259, SEQ ID NO: 6260, and SEQ ID NO: 6261, respectively.

138. The composition of claim 136, wherein the VHCDR1, VHCDR2, VHCDR3, VLCDR1, VLCDR2, and VLCDR3 of the first antigen binding domain comprise the sequences of:
SEQ ID NOs: 6253, 6254, 6255, 6259, 6260, and 6261, respectively;
SEQ ID NO: 6256, 6257, 6258, 6259, 6260, and 6261 respectively;
SEQ ID NOs: 6253, 6254, 6255, 6259, 6260, and 6261 respectively;
SEQ ID NO: 6256, 6257, 6262, 6259, 6260, and 6261 respectively;
SEQ ID NO: 6358, 6360, 227, 251, 246, and 248 respectively;
SEQ ID NO: 6358, 6360, 227, 251, 253, and 255 respectively;
SEQ ID NO: 6358, 6360, 227, 258, 260, and 262 respectively;
SEQ ID NO: 6358, 6360, 227, 258, 260, and 248 respectively;
SEQ ID NO: 6358, 6360, 227, 265, 267, 269 and respectively; or
SEQ ID NO: 7396, 7400, 7403, 7387, 7389, and 7392, respectively.

139. The composition of claim 136, wherein:
(a) (i) the VH of the first antigen binding domain comprises a sequence having at least 75% identity to the sequence of: SEQ ID NO: 6247, SEQ ID NO: 6248, SEQ ID NO: 6250, SEQ ID NO: 6251, SEQ ID NO: 6347, SEQ ID NO: 7418, SEQ ID NO: 7425, SEQ ID NO: 7438, SEQ ID NO: 7446, SEQ ID NO: 6349, SEQ ID NO: 6350, SEQ ID NO: 6351, SEQ ID NO:6372, SEQ ID NO: 234, SEQ ID NO: 235, SEQ ID NO: 236, or SEQ ID NO: 237; and
(ii) the VL of the first antigen binding domain comprises a sequence having at least 75% identity to the sequence of: SEQ ID NO: 6249, SEQ ID NO: 6252, SEQ ID NO: 6348, SEQ ID NO: 6352, SEQ ID NO:6353, SEQ ID NO: 6354, SEQ ID NO: 6355, SEQ ID NO: 6356, SEQ ID NO: 7419, SEQ ID NO: 7420, SEQ ID NO: 7421, SEQ ID NO: 7422, SEQ ID NO: 7428, SEQ ID NO: 7432, SEQ ID NO: 7435, SEQ ID NO: 7440, SEQ ID NO: 7441, SEQ ID NO: 7442, 238, SEQ ID NO: 239, SEQ ID NO:240, SEQ ID NO: 241, or SEQ ID NO: 242;
(b) the VH and the VL of the first antigen binding domain comprise sequences having at least 75% identity to the sequences of:
SEQ ID NO: 6372 and SEQ ID NO: 238, respectively;
SEQ ID NO: 234 and SEQ ID NO: 238, respectively;
SEQ ID NO: 235 and SEQ ID NO: 238, respectively;
SEQ ID NO: 236 and SEQ ID NO: 238, respectively;
SEQ ID NO: 237 and SEQ ID NO: 238, respectively;
SEQ ID NO: 6372 and SEQ ID NO: 239, respectively;
SEQ ID NO: 234 and SEQ ID NO: 239, respectively;
SEQ ID NO: 235 and SEQ ID NO: 239, respectively;
SEQ ID NO: 236 and SEQ ID NO: 239, respectively;
SEQ ID NO: 237 and SEQ ID NO: 239, respectively;
SEQ ID NO: 6372 and SEQ ID NO: 240, respectively;
SEQ ID NO: 234 and SEQ ID NO: 240, respectively;
SEQ ID NO: 235 and SEQ ID NO: 240, respectively;
SEQ ID NO: 236 and SEQ ID NO: 240, respectively;
SEQ ID NO: 237 and SEQ ID NO: 240, respectively;
SEQ ID NO: 6372 and SEQ ID NO: 241, respectively;
SEQ ID NO: 234 and SEQ ID NO: 241, respectively;
SEQ ID NO: 235 and SEQ ID NO: 241, respectively;
SEQ ID NO: 236 and SEQ ID NO: 241, respectively;
SEQ ID NO: 237 and SEQ ID NO: 241, respectively;
SEQ ID NO: 6372 and SEQ ID NO: 242, respectively;
SEQ ID NO: 234 and SEQ ID NO: 242, respectively;
SEQ ID NO: 234 and SEQ ID NO: 242, respectively;
SEQ ID NO: 235 and SEQ ID NO: 242, respectively;
SEQ ID NO: 236 and SEQ ID NO: 242, respectively; or
SEQ ID NO: 237 and SEQ ID NO: 242, respectively; or
(c) the first antigen binding domain comprises a sequence having at least 75% identity to the sequence of: SEQ ID NO: 7499, SEQ ID NO: 7500, SEQ ID NO: 7501, SEQ ID NO: 7502, SEQ ID NO: 7503, SEQ ID NO: 7504, SEQ ID NO: 7505, or SEQ ID NO: 7506.

140. The composition of claim 136, wherein:
(a) (i) the VH of the first antigen binding domain comprises the sequence of: SEQ ID NO: 6247, SEQ ID NO: 6248, SEQ ID NO: 6250, SEQ ID NO: 6251, SEQ ID NO: 6347, SEQ ID NO: 7418, SEQ ID NO: 7425, SEQ ID NO: 7438, SEQ ID NO: 7446, SEQ ID NO: 6349, SEQ ID NO: 6350, SEQ ID NO: 6351, SEQ ID NO:6372, SEQ ID NO: 234, SEQ ID NO: 235, SEQ ID NO: 236, or SEQ ID NO: 237; and
(ii) the VL of the first antigen binding domain comprises the sequence of: SEQ ID NO: 6249, SEQ ID NO: 6252, SEQ ID NO: 6348, SEQ ID NO: 6352, SEQ ID NO:6353, SEQ ID NO: 6354, SEQ ID NO: 6355, SEQ ID NO: 6356, SEQ ID NO: 7419, SEQ ID NO: 7420, SEQ ID NO: 7421, SEQ ID NO: 7422, SEQ ID NO: 7428, SEQ ID NO: 7432, SEQ ID NO: 7435, SEQ ID NO: 7440, SEQ ID NO: 7441, SEQ ID NO: 7442, 238, SEQ ID NO: 239, SEQ ID NO:240, SEQ ID NO: 241, or SEQ ID NO: 242;
(b) the VH and the VL of the first antigen binding domain comprise the sequences of:
SEQ ID NO: 6372 and SEQ ID NO: 238, respectively;
SEQ ID NO: 234 and SEQ ID NO: 238, respectively;
SEQ ID NO: 235 and SEQ ID NO: 238, respectively;
SEQ ID NO: 236 and SEQ ID NO: 238, respectively;
SEQ ID NO: 237 and SEQ ID NO: 238, respectively;
SEQ ID NO: 6372 and SEQ ID NO: 239, respectively;
SEQ ID NO: 234 and SEQ ID NO: 239, respectively;
SEQ ID NO: 235 and SEQ ID NO: 239, respectively;
SEQ ID NO: 236 and SEQ ID NO: 239, respectively;
SEQ ID NO: 237 and SEQ ID NO: 239, respectively;
SEQ ID NO: 6372 and SEQ ID NO: 240, respectively;
SEQ ID NO: 234 and SEQ ID NO: 240, respectively;
SEQ ID NO: 235 and SEQ ID NO: 240, respectively;
SEQ ID NO: 236 and SEQ ID NO: 240, respectively;
SEQ ID NO: 237 and SEQ ID NO: 240, respectively;
SEQ ID NO: 6372 and SEQ ID NO: 241, respectively;
SEQ ID NO: 234 and SEQ ID NO: 241, respectively;
SEQ ID NO: 235 and SEQ ID NO: 241, respectively;
SEQ ID NO: 236 and SEQ ID NO: 241, respectively;
SEQ ID NO: 237 and SEQ ID NO: 241, respectively;
SEQ ID NO: 6372 and SEQ ID NO: 242, respectively;
SEQ ID NO: 234 and SEQ ID NO: 242, respectively;
SEQ ID NO: 234 and SEQ ID NO: 242, respectively;
SEQ ID NO: 235 and SEQ ID NO: 242, respectively;
SEQ ID NO: 236 and SEQ ID NO: 242, respectively; or
SEQ ID NO: 237 and SEQ ID NO: 242, respectively; or
(c) the first antigen binding domain comprises the sequence of: SEQ ID NO: 7499, SEQ ID NO: 7500, SEQ ID NO: 7501, SEQ ID NO: 7502, SEQ ID NO: 7503, SEQ ID NO: 7504, SEQ ID NO: 7505, or SEQ ID NO: 7506.

141. The composition of claim 136, wherein the second antigen binding domain binds to TCRβV and comprises:
(a) the VHCDR1, VHCDR2, and VHCDR3 of the second antigen binding domain comprising the sequences of:
SEQ ID NO: 3, SEQ ID NO: 4, and SEQ ID NO: 5, respectively;
SEQ ID NO: 45, SEQ ID NO: 46, and SEQ ID NO: 47, respectively;
SEQ ID NO: 48, SEQ ID NO: 49, and SEQ ID NO: 50, respectively;
SEQ ID NO: 17, SEQ ID NO: 18, and SEQ ID NO: 19, respectively;
SEQ ID NO: 57, SEQ ID NO: 58, and SEQ ID NO: 59, respectively;
SEQ ID NO: 60, SEQ ID NO: 61, and SEQ ID NO: 62, respectively;
SEQ ID NO: 1315, SEQ ID NO: 1316, SEQ ID NO: 1317, respectively;
SEQ ID NO: 1318, SEQ ID NO: 1319, SEQ ID NO: 1317, respectively;
SEQ ID NO: 1320, SEQ ID NO: 1316, SEQ ID NO: 1317, respectively;
SEQ ID NO: 1298, SEQ ID NO: 1299, and SEQ ID NO: 1300, respectively;
SEQ ID NO: 1302, SEQ ID NO: 1303, and SEQ ID NO: 1301, respectively;
SEQ ID NO: 1304, SEQ ID NO: 1299, and SEQ ID NO: 1301, respectively;
SEQ ID NO: 1288, SEQ ID NO: 1289, and SEQ ID NO: 1290, respectively;
SEQ ID NO: 1291, SEQ ID NO: 1292, and SEQ ID NO: 1290, respectively;
SEQ ID NO: 1293, SEQ ID NO: 1289, and SEQ ID NO: 1290, respectively;
SEQ ID NO: 1102, SEQ ID NO: 1103, and SEQ ID NO: 1104, respectively;
SEQ ID NO: 1105, SEQ ID NO: 1106, and SEQ ID NO: 1104, respectively;
SEQ ID NO: 1107, SEQ ID NO: 1103, and SEQ ID NO: 1104, respectively;
SEQ ID NO: 1120, SEQ ID NO: 1121, and SEQ ID NO: 1122, respectively;
SEQ ID NO: 1123, SEQ ID NO: 1124, and SEQ ID NO: 1122, respectively;
SEQ ID NO: 1123, SEQ ID NO: 1124, and SEQ ID NO: 1122, respectively;
SEQ ID NO: 1125, SEQ ID NO: 1121, and SEQ ID NO: 1122, respectively;
SEQ ID NO: 1141, SEQ ID NO: 1142, and SEQ ID NO: 1143, respectively;
SEQ ID NO: 1144, SEQ ID NO: 1145, and SEQ ID NO: 1143, respectively;
SEQ ID NO: 1146, SEQ ID NO: 1142, and SEQ ID NO: 1143, respectively;
SEQ ID NO: 1163, SEQ ID NO: 1164, and SEQ ID NO: 1165, respectively;
SEQ ID NO: 1166, SEQ ID NO: 1167, and SEQ ID NO: 1165, respectively;
SEQ ID NO: 1166, SEQ ID NO: 1164, and SEQ ID NO: 1165, respectively;
SEQ ID NO: 1185, SEQ ID NO: 1186, and SEQ ID NO: 1187, respectively;
SEQ ID NO: 1188, SEQ ID NO: 1189, and SEQ ID NO: 1187, respectively;
SEQ ID NO: 1190, SEQ ID NO: 1186, and SEQ ID NO: 1187, respectively;
SEQ ID NO: 1208, SEQ ID NO: 1209, and SEQ ID NO: 1210, respectively;
SEQ ID NO: 1211, SEQ ID NO: 1212, and SEQ ID NO: 1210, respectively;
SEQ ID NO: 1213, SEQ ID NO: 1209, and SEQ ID NO: 1210, respectively;
SEQ ID NO: 1229, SEQ ID NO: 1230, and SEQ ID NO: 1231, respectively;
SEQ ID NO: 1232, SEQ ID NO: 1233, and SEQ ID NO: 1231, respectively;
SEQ ID NO: 1234, SEQ ID NO: 1230, and SEQ ID NO: 1231, respectively;
SEQ ID NO: 1248, SEQ ID NO: 1249, and SEQ ID NO: 1250, respectively;
SEQ ID NO: 1251, SEQ ID NO: 1252, and SEQ ID NO: 1250, respectively;
SEQ ID NO: 1253, SEQ ID NO: 1249, and SEQ ID NO: 1250, respectively;
SEQ ID NO: 1268, SEQ ID NO: 1269, and SEQ ID NO: 1270, respectively;
SEQ ID NO: 1271, SEQ ID NO: 1272, and SEQ ID NO: 1270, respectively; or
SEQ ID NO: 1273, SEQ ID NO: 1269, and SEQ ID NO: 1270, respectively; and
(b) the VLCDR1, VLCDR2, and VLCDR3 of the second antigen binding domain comprising the sequences of:
SEQ ID NO: 6, SEQ ID NO: 7, and SEQ ID NO: 8, respectively;
SEQ ID NO: 51, SEQ ID NO: 52, and SEQ ID NO: 53, respectively;
SEQ ID NO: 54, SEQ ID NO: 55, and SEQ ID NO: 56, respectively;
SEQ ID NO: 20, SEQ ID NO: 21, and SEQ ID NO: 22, respectively;
SEQ ID NO: 63, SEQ ID NO: 64, and SEQ ID NO: 65, respectively;
SEQ ID NO: 66, SEQ ID NO: 67, and SEQ ID NO: 68, respectively;
SEQ ID NO: 1305, SEQ ID NO: 1306, and SEQ ID NO: 1307, respectively;
SEQ ID NO: 1308, SEQ ID NO: 1306, and SEQ ID NO: 1307, respectively;
SEQ ID NO: 1294, SEQ ID NO: 1295, and SEQ ID NO: 1296, respectively;
SEQ ID NO: 1297, SEQ ID NO: 1295, and SEQ ID NO: 1296, respectively;
SEQ ID NO: 7489, SEQ ID NO: 1108, and SEQ ID NO: 1109, respectively;
SEQ ID NO: 1110, SEQ ID NO: 1108, and SEQ ID NO: 1109, respectively;
SEQ ID NO: 1126, SEQ ID NO: 1127, and SEQ ID NO: 1128, respectively;
SEQ ID NO: 1129, SEQ ID NO: 1127, and SEQ ID NO: 1128, respectively;
SEQ ID NO: 1147, SEQ ID NO: 1148, and SEQ ID NO: 1149, respectively;
SEQ ID NO: 1150, SEQ ID NO: 1148, and SEQ ID NO: 1149, respectively;
SEQ ID NO: 1168, SEQ ID NO: 1169, and SEQ ID NO: 1170, respectively;
SEQ ID NO: 1171, SEQ ID NO: 1169, and SEQ ID NO: 1170, respectively;
SEQ ID NO: 1191, SEQ ID NO: 1192, and SEQ ID NO: 1193, respectively;
SEQ ID NO: 1194, SEQ ID NO: 1192, and SEQ ID NO: 1193, respectively;
SEQ ID NO: 1214, SEQ ID NO: 1215, and SEQ ID NO: 1216, respectively;
SEQ ID NO: 1217, SEQ ID NO: 1215, and SEQ ID NO: 1216, respectively;
SEQ ID NO: 1235, SEQ ID NO: 1236, and SEQ ID NO: 1237, respectively;
SEQ ID NO: 1238, SEQ ID NO: 1236, and SEQ ID NO: 1237, respectively;
SEQ ID NO: 1254, SEQ ID NO: 1255, and SEQ ID NO: 1256, respectively;
SEQ ID NO: 1257, SEQ ID NO: 1255, and SEQ ID NO: 1256, respectively; or
SEQ ID NO: 1275, SEQ ID NO: 1276, and SEQ ID NO: 1277, respectively.

142. The composition of claim 136, wherein the second antigen binding domain binds to TCRβV, and wherein the VHCDR1, VHCDR2, a VHCDR3, VLCDR1, VLCDR2, and VLCDR3 of the second antigen binding domain comprise the sequences of:
SEQ ID NOs: 3, 4, 5, 6, 7, and 8, respectively;
SEQ ID NOs: 45, 46, 47, 51, 52, and 53, respectively;
SEQ ID NOs: 48, 49, 50, 54, 55, and 56, respectively;
SEQ ID NOs: 17, 18, 19, 20, 21, and 22, respectively;
SEQ ID NOs: 57, 58, 59, 63, 64, and 65, respectively;
SEQ ID NOs: 60, 61, 62, 66, 67, and 68, respectively;
SEQ ID NOs: 1315, 1316, 1317, 1321, 1322, and 1323, respectively;
SEQ ID NOs: 1318, 1319, 1317, 1321, 1322, and 1323, respectively;
SEQ ID NOs: 1320, 1316, 1317, 1321, 1322, and 1323, respectively;
SEQ ID NOs: 1298, 1299, 1300, 1305, 1306, and 1307, respectively;
SEQ ID NOs: 1302, 1303, 1301, 1308, 1306, and 1307, respectively;
SEQ ID NOs: 1304, 1299, 1301, 1305, 1306, and 1307, respectively;
SEQ ID NOs: 1288, 1289, 1290, 1294, 1295, and 1296, respectively;
SEQ ID NOs: 1291, 1292, 1290, 1297, 1295, and 1296, respectively;
SEQ ID NOs: 1293, 1289, 1290, 1294, 1295, and 1296, respectively;
SEQ ID NOs: 1102, 1103, 1104, 7489, 1108, and 1109, respectively;
SEQ ID NOs: 1105, 1106, 1104, 1110, 1108, and 1109, respectively;
SEQ ID NOs: 1107, 1103, 1104, 7489, 1108, and 1109, respectively;
SEQ ID NOs: 1120, 1121, 1122, 1126, 1127, and 1128, respectively;
SEQ ID NOs: 1123, 1124, 1122, 1129, 1127, and 1128, respectively;
SEQ ID NOs: 1125, 1121, 1122, 1126, 1127, and 1128, respectively;
SEQ ID NOs: 1141, 1142, 1143, 1147, 1148, and 1149, respectively;
SEQ ID NOs: 1144, 1145, 1143, 1150, 1148, and 1149, respectively;
SEQ ID NOs: 1146, 1142, 1143, 1147, 1148, and 1149, respectively;
SEQ ID NOs: 1163, 1164, 1165, 1168, 1169, and 1170, respectively;
SEQ ID NOs: 1166, 1167, 1165, 1171, 1169, and 1170, respectively;
SEQ ID NOs: 1166, 1164, 1165, 1168, 1169, and 1170, respectively;
SEQ ID NOs: 1185, 1186, 1187, 1191, 1192, and 1193, respectively;
SEQ ID NOs: 1188, 1189, 1187, 1194, 1192, and 1193, respectively;
SEQ ID NOs: 1190, 1186, 1187, 1191, 1192, and 1193, respectively;
SEQ ID NOs: 1208, 1209, 1210, 1214, 1215, and 1216, respectively;
SEQ ID NOs: 1211, 1212 1210, 1217, 1215, and 1216, respectively;
SEQ ID NOs: 1213, 1209, 1210, 1214, 1215, and 1216, respectively;
SEQ ID NOs: 1229, 1230, 1231, 1235, 1236, and 1237, respectively;
SEQ ID NOs: 1232, 1233, 1231, 1238, 1236, and 1237, respectively;
SEQ ID NOs: 1234, 1230, 1231, 1235, 1236, and 1237, respectively;
SEQ ID NOs: 1248, 1249, 1250, 1254, 1255, and 1256, respectively;
SEQ ID NOs: 1251, 1252, 1250, 1257, 1255, and 1256, respectively;
SEQ ID NOs: 1253, 1249, 1250, 1254, 1255, and 1256, respectively;
SEQ ID NOs: 1268, 1269, 1270, 1275, 1276, and 1277, respectively;
SEQ ID NOs: 1271, 1272, 1270, 1275, 1276, and 1277, respectively; or
SEQ ID NOs: 1273, 1269, 1270, 1275, 1276, and 1277, respectively.

143. The composition of claim 136, wherein the second antigen binding domain binds to TCRβV, and wherein:
(a) (i) the VH of the second antigen binding domain comprises a sequence having at least 75% identity to SEQ ID NO: 1, SEQ ID NO: 9, SEQ ID NO: 82, SEQ ID NO: 85, SEQ ID NO: 88, SEQ ID NO: 91, SEQ ID NO: 94, SEQ ID NO: 97, SEQ ID NO: 100, SEQ ID NO: 103, SEQ ID NO: 106, SEQ ID NO: 109, SEQ ID NO: 112, SEQ ID NO: 115, SEQ ID NO: 118, SEQ ID NO: 121, SEQ ID NO: 124, SEQ ID NO: 127, SEQ ID NO: 130, SEQ ID NO: 133, SEQ ID NO: 136, SEQ ID NO: 139, SEQ ID NO: 142, SEQ ID NO: 145, SEQ ID NO: 148, SEQ ID NO: 151, SEQ ID NO: 155, SEQ ID NO: 158, SEQ ID NO: 161, SEQ ID NO: 164, SEQ ID NO: 167, SEQ ID NO: 170, SEQ ID NO: 173, SEQ ID NO: 176, SEQ ID NO: 179, SEQ ID NO: 182, SEQ ID NO: 185, SEQ ID NO: 188, SEQ ID NO: 191, SEQ ID NO: 194, SEQ ID NO: 197, SEQ ID NO: 200, SEQ ID NO: 203, SEQ ID NO: 205, SEQ ID NO: 207, SEQ ID NO: 209, SEQ ID NO: 211, SEQ ID NO: 213, SEQ ID NO: 215, SEQ ID NO:217, SEQ ID NO: 219, SEQ ID NO: 221, SEQ ID NO: 223, SEQ ID NO: 225, SEQ ID NO: 1100, SEQ ID NO: 1310, SEQ ID NO:1311, SEQ ID NO: 1312, SEQ ID NO: 1346, SEQ ID NO:1348, SEQ ID NO: 1350, SEQ ID NO: 1356, SEQ ID NO: 1360, SEQ ID NO: 1362, SEQ ID NO: 1370, SEQ ID NO: 1344, SEQ ID NO: 15, SEQ ID NO: 23, SEQ ID NO: 24, SEQ ID NO: 25, SEQ ID NO: 232, SEQ ID NO: 1325, SEQ ID NOs: 3040-3183, SEQ ID NOs: 3225-3274, SEQ ID NOs: 1112-1116, SEQ ID NOs: 1130-1135, SEQ ID NOs: 1152-1157, SEQ ID NOs: 1172-1177, SEQ ID NOs: 1196-1202, SEQ ID NOs: 1219-1223, SEQ ID NOs: 1240-1243, SEQ ID NOs: 1259-1263, SEQ ID NOs: 1283-1287, or SEQ ID NO: 1274; and
(ii) the VL of the second antigen binding domain comprises a sequence having at least 75% identity to the sequence of:
SEQ ID NO: 2, SEQ ID NO: 10, SEQ ID NO: 11, SEQ ID NO: 81, SEQ ID NO: 84, SEQ ID NO: 87, SEQ ID NO: 90, SEQ ID NO: 93, SEQ ID NO: 96, SEQ ID NO: 99, SEQ ID NO: 102, SEQ ID NO: 105, SEQ ID NO: 108, SEQ ID NO: 111, SEQ ID NO: 114, SEQ ID NO: 117, SEQ ID NO: 120, SEQ ID NO: 123, SEQ ID NO: 126, SEQ ID NO: 129, SEQ ID NO: 132, SEQ ID NO: 135, SEQ ID NO: 138, SEQ ID NO: 141, SEQ ID NO: 144, SEQ ID NO: 147, SEQ ID NO: 150, SEQ ID NO: 154, SEQ ID NO: 157, SEQ ID NO: 160, SEQ ID NO: 163, SEQ ID NO: 166, SEQ ID NO: 169, SEQ ID NO: 172, SEQ ID NO: 175, SEQ ID NO: 178, SEQ ID NO: 181, SEQ ID NO: 184, SEQ ID NO: 187, SEQ ID NO: 190, SEQ ID NO: 193, SEQ ID NO: 196, SEQ ID NO: 199, SEQ ID NO: 202, SEQ ID NO: 1101, SEQ ID NO: 1313, SEQ ID NO: 1314, SEQ ID NO: 1347, SEQ ID NO: 1349, SEQ ID NO: 1351, SEQ ID NO: 1353, SEQ ID NO: 1357, SEQ ID NO: 1361, SEQ ID NO: 1365, SEQ ID NO: 1367, SEQ ID NO: 1369, SEQ ID NO: 16, SEQ ID NOs: 26-30, SEQ ID NOs: 3000-3039, SEQ ID NOs: 3092-3132, SEQ ID NOs: 3184-3224, SEQ ID NOs: 1117-1119, SEQ ID NOs: 1136-1140, SEQ ID NOs: 1158-1162, SEQ ID NOs: 1178-1184, SEQ ID NOs: 1203-1207, SEQ ID NOs: 1224-1227, SEQ ID NOs: 1244-1247, SEQ ID NOs: 1264-1267, SEQ ID NOs: 1278-1282, SEQ ID NO: 7488, SEQ ID NO: 1324, SEQ ID NO: 1111, SEQ ID NO: 1151, SEQ ID NO: 1195, SEQ ID NO: 1218, SEQ ID NO: 1239, SEQ ID NO: 1258, SEQ ID NO: 7490, or SEQ ID NO: 7491;
(b) the VH and the VL of the second antigen binding domain comprise sequences having at least 75% identity to the sequences of:
SEQ ID NO: 1 and SEQ ID NO: 2, respectively;
SEQ ID NO: 9 and SEQ ID NO: 10, respectively;
SEQ ID NO: 9 and SEQ ID NO: 11, respectively;
SEQ ID NO: 82 and SEQ ID NO: 81, respectively;
SEQ ID NO: 85 and SEQ ID NO: 84, respectively;
SEQ ID NO: 88 and SEQ ID NO: 87, respectively;
SEQ ID NO: 91 and SEQ ID NO: 90, respectively;
SEQ ID NO: 94 and SEQ ID NO: 93, respectively;
SEQ ID NO: 97 and SEQ ID NO: 96, respectively;
SEQ ID NO: 100 and SEQ ID NO: 99, respectively;
SEQ ID NO: 103 and SEQ ID NO: 102, respectively;
SEQ ID NO: 106 and SEQ ID NO: 105, respectively;
SEQ ID NO: 109 and SEQ ID NO: 108, respectively;
SEQ ID NO: 112 and SEQ ID NO: 111, respectively;
SEQ ID NO: 115 and SEQ ID NO: 114, respectively;
SEQ ID NO: 118 and SEQ ID NO: 117, respectively;
SEQ ID NO: 121 and SEQ ID NO: 120, respectively;
SEQ ID NO: 124 and SEQ ID NO: 123, respectively;
SEQ ID NO: 127 and SEQ ID NO: 126, respectively;
SEQ ID NO: 130 and SEQ ID NO: 129, respectively;
SEQ ID NO: 133 and SEQ ID NO: 132, respectively;
SEQ ID NO: 136 and SEQ ID NO: 135, respectively;
SEQ ID NO: 139 and SEQ ID NO: 138, respectively;
SEQ ID NO: 142 and SEQ ID NO: 141, respectively;
SEQ ID NO: 145 and SEQ ID NO: 144, respectively;
SEQ ID NO: 148 and SEQ ID NO: 147, respectively;
SEQ ID NO: 151 and SEQ ID NO: 150, respectively;
SEQ ID NO: 155 and SEQ ID NO: 154, respectively;
SEQ ID NO: 158 and SEQ ID NO: 157, respectively;
SEQ ID NO: 161 and SEQ ID NO: 160, respectively;
SEQ ID NO: 164 and SEQ ID NO: 163, respectively;
SEQ ID NO: 167 and SEQ ID NO: 166, respectively;
SEQ ID NO: 170 and SEQ ID NO: 169, respectively;
SEQ ID NO: 173 and SEQ ID NO: 172, respectively;
SEQ ID NO: 176 and SEQ ID NO: 175, respectively;
SEQ ID NO: 179 and SEQ ID NO: 178, respectively;
SEQ ID NO: 182 and SEQ ID NO: 181, respectively;
SEQ ID NO: 185 and SEQ ID NO: 184, respectively;
SEQ ID NO: 188 and SEQ ID NO: 187, respectively;
SEQ ID NO: 191 and SEQ ID NO: 190, respectively;
SEQ ID NO: 194 and SEQ ID NO: 193, respectively;
SEQ ID NO: 197 and SEQ ID NO: 196, respectively;
SEQ ID NO: 200 and SEQ ID NO: 199, respectively;
SEQ ID NO: 203 and SEQ ID NO: 202, respectively;
SEQ ID NO: 1100 and SEQ ID NO: 1101, respectively;
SEQ ID NO: 1346 and SEQ ID NO: 1347, respectively;
SEQ ID NO: 1348 and SEQ ID NO: 1349, respectively;
SEQ ID NO: 1350 and SEQ ID NO: 1351, respectively;
SEQ ID NO: 1350 and SEQ ID NO: 1353, respectively;
SEQ ID NO: 1346 and SEQ ID NO: 1349, respectively;
SEQ ID NO: 1356 and SEQ ID NO: 1357, respectively;
SEQ ID NO: 1350 and SEQ ID NO: 1349, respectively;
SEQ ID NO: 1360 and SEQ ID NO: 1361, respectively;
SEQ ID NO: 1362 and SEQ ID NO: 1361, respectively;
SEQ ID NO: 1350 and SEQ ID NO: 1365, respectively;
SEQ ID NO: 1350 and SEQ ID NO: 1367, respectively;
SEQ ID NO: 1350 and SEQ ID NO: 1369, respectively;
SEQ ID NO: 1370 and SEQ ID NO: 1365, respectively;
SEQ ID NO: 1370 and SEQ ID NO: 1367, respectively;
SEQ ID NO: 1370 and SEQ ID NO: 1369, respectively;
SEQ ID NO: 1344 and SEQ ID NO: 1361, respectively;
SEQ ID NO: 15 and SEQ ID NO: 16, respectively;
SEQ ID NO: 23 and SEQ ID NO: 26, respectively;
SEQ ID NO: 24 and SEQ ID NO: 27, respectively;
SEQ ID NO: 25 and SEQ ID NO: 28, respectively;
SEQ ID NO: 232 and SEQ ID NO: 7488, respectively;
SEQ ID NO: 1325 and SEQ ID NO: 1324, respectively;
SEQ ID NO: 3091 and SEQ ID NO: 3092, respectively;
SEQ ID NO: 3183 and SEQ ID NO: 3184, respectively;
SEQ ID NO: 1112 and SEQ ID NO: 1111, respectively;
SEQ ID NO: 1130 and SEQ ID NO: 7490, respectively;
SEQ ID NO: 1152 and SEQ ID NO: 1151, respectively;
SEQ ID NO: 1172 and SEQ ID NO: 7491, respectively;
SEQ ID NO: 1196 and SEQ ID NO: 1195, respectively;
SEQ ID NO: 1219 and SEQ ID NO: 1218, respectively;
SEQ ID NO: 1240 and SEQ ID NO: 1239, respectively;
SEQ ID NO: 1259 and SEQ ID NO: 1258, respectively; or
SEQ ID NO: 1274 and SEQ ID NO: 1278, respectively; or
(c) the second antigen binding domain comprises a sequence having at least 75% identity to the sequence of: SEQ ID NO: 80, SEQ ID NO: 83, SEQ ID NO: 86, SEQ ID NO: 89, SEQ ID NO: 92, SEQ ID NO: 95, SEQ ID NO: 98, SEQ ID NO: 101, SEQ ID NO: 104, SEQ ID NO: 107, SEQ ID NO: 110, SEQ ID NO: 113, SEQ ID NO: 116, SEQ ID NO: 119, SEQ ID NO: 122, SEQ ID NO: 125, SEQ ID NO: 128, SEQ ID NO: 131, SEQ ID NO: 134, SEQ ID NO: 137, SEQ ID NO: 140, SEQ ID NO: 143, SEQ ID NO: 146, SEQ ID NO: 149, SEQ ID NO: 153, SEQ ID NO: 156, SEQ ID NO: 159, SEQ ID NO: 162, SEQ ID NO: 165, SEQ ID NO: 168, SEQ ID NO: 171, SEQ ID NO: 174, SEQ ID NO: 177, SEQ ID NO: 180, SEQ ID NO: 183, SEQ ID NO: 186, SEQ ID NO: 189, SEQ ID NO: 192, SEQ ID NO: 195, SEQ ID NO: 198, SEQ ID NO: 201, SEQ ID NO: 204, SEQ ID NO: 206, SEQ ID NO: 208, SEQ ID NO: 210, SEQ ID NO: 212, SEQ ID NO: 214, SEQ ID NO: 216, SEQ ID NO: 218, SEQ ID NO: 220, SEQ ID NO: 222, SEQ ID NO: 224, SEQ ID NO: 1309, SEQ ID NO: 3281, SEQ ID NO: 7492 SEQ ID NO: 1326-1342, or SEQ ID NO: 1376.

144. The composition of claim 136, wherein the second antigen binding domain binds to TCRβV, and wherein:
(a) (i) the VH of the second antigen binding domain comprises the sequence of: SEQ ID NO: 1, SEQ ID NO: 9, SEQ ID NO: 82, SEQ ID NO: 85, SEQ ID NO: 88, SEQ ID NO: 91, SEQ ID NO: 94, SEQ ID NO: 97, SEQ ID NO: 100, SEQ ID NO: 103, SEQ ID NO: 106, SEQ ID NO: 109, SEQ ID NO: 112, SEQ ID NO: 115, SEQ ID NO: 118, SEQ ID NO: 121, SEQ ID NO: 124, SEQ ID NO: 127, SEQ ID NO: 130, SEQ ID NO: 133, SEQ ID NO: 136, SEQ ID NO: 139, SEQ ID NO: 142, SEQ ID NO: 145, SEQ ID NO: 148, SEQ ID NO: 151, SEQ ID NO: 155, SEQ ID NO: 158, SEQ ID NO: 161, SEQ ID NO: 164, SEQ ID NO: 167, SEQ ID NO: 170, SEQ ID NO: 173, SEQ ID NO: 176, SEQ ID NO: 179, SEQ ID NO: 182, SEQ ID NO: 185, SEQ ID NO: 188, SEQ ID NO: 191, SEQ ID NO: 194, SEQ ID NO: 197, SEQ ID NO: 200, SEQ ID NO: 203, SEQ ID NO: 205, SEQ ID NO: 207, SEQ ID NO: 209, SEQ ID NO: 211, SEQ ID NO: 213, SEQ ID NO: 215, SEQ ID NO:217, SEQ ID NO: 219, SEQ ID NO: 221, SEQ ID NO: 223, SEQ ID NO: 225, SEQ ID NO: 1100, SEQ ID NO: 1310, SEQ ID NO:1311, SEQ ID NO: 1312, SEQ ID NO: 1346, SEQ ID NO:1348, SEQ ID NO: 1350, SEQ ID NO: 1356, SEQ ID NO: 1360, SEQ ID NO: 1362, SEQ ID NO: 1370, SEQ ID NO: 1344, SEQ ID NO: 15, SEQ ID NO: 23, SEQ ID NO: 24, SEQ ID NO: 25, SEQ ID NO: 232, SEQ ID NO: 1325, SEQ ID NOs: 3040-3183, SEQ ID NOs: 3225-3274, SEQ ID NOs: 1112-1116, SEQ ID NOs: 1130-1135, SEQ ID NOs: 1152-1157, 1172-1177, SEQ ID NOs: 1196-1202, SEQ ID NOs: 1219-1223, SEQ ID NOs: 1240-1243, SEQ ID NOs: 1259-1263, SEQ ID NOs: 1284-1287, or SEQ ID NO: 1274; and
(ii) the VL of the second antigen binding domain comprises the sequence of: SEQ ID NO: 2, SEQ ID NO: 10, SEQ ID NO: 11, SEQ ID NO: 81, SEQ ID NO: 84, SEQ ID NO: 87, SEQ ID NO: 90, SEQ ID NO: 93, SEQ ID NO: 96, SEQ ID NO: 99, SEQ ID NO: 102, SEQ ID NO: 105, SEQ ID NO: 108, SEQ ID NO: 111, SEQ ID NO: 114, SEQ ID NO: 117, SEQ ID NO: 120, SEQ ID NO: 123, SEQ ID NO: 126, SEQ ID NO: 129, SEQ ID NO: 132, SEQ ID NO: 135, SEQ ID NO: 138, SEQ ID NO: 141, SEQ ID NO: 144, SEQ ID NO: 147, SEQ ID NO: 150, SEQ ID NO: 154, SEQ ID NO: 157, SEQ ID NO: 160, SEQ ID NO: 163, SEQ ID NO: 166, SEQ ID NO: 169, SEQ ID NO: 172, SEQ ID NO: 175, SEQ ID NO: 178, SEQ ID NO: 181, SEQ ID NO: 184, SEQ ID NO: 187, SEQ ID NO: 190, SEQ ID NO: 193, SEQ ID NO: 196, SEQ ID NO: 199, SEQ ID NO: 202, SEQ ID NO: 1101, SEQ ID NO: 1313, SEQ ID NO: 1314, SEQ ID NO: 1347, SEQ ID NO: 1349, SEQ ID NO: 1351, SEQ ID NO: 1353, SEQ ID NO: 1357, SEQ ID NO: 1361, SEQ ID NO: 1365, SEQ ID NO: 1367, SEQ ID NO: 1369, SEQ ID NO: 16, SEQ ID NOs: 26-30, SEQ ID NOs: 3000-3039, SEQ ID NOs: 3092-3132, SEQ ID NOs: 3184-3224, SEQ ID NOs: 1117-1119, SEQ ID NOs: 1136-1140, SEQ ID NOs: 1158-1162, SEQ ID NOs: 1178-1184, SEQ ID NOs: 1203-1207, SEQ ID NOs: 1224-1227, SEQ ID NOs: 1244-1247, SEQ ID NOs: 1264-1267, SEQ ID NOs: 1278-1282, SEQ ID NOs: SEQ ID NO: 7488, SEQ ID NO: 1324, SEQ ID NO: 1111, SEQ ID NO: 1151, SEQ ID NO: 1195, SEQ ID NO: 1218, SEQ ID NO: 1239, SEQ ID NO: 1258, SEQ ID NO: 7490, or 7491;
(b) the VH and the VL of the second antigen binding domain comprise the sequences of:
SEQ ID NO: 1 and SEQ ID NO: 2, respectively;
SEQ ID NO: 9 and SEQ ID NO: 10, respectively;
SEQ ID NO: 9 and SEQ ID NO: 11, respectively;
SEQ ID NO: 82 and SEQ ID NO: 81, respectively;
SEQ ID NO: 85 and SEQ ID NO: 84, respectively;
SEQ ID NO: 88 and SEQ ID NO: 87, respectively;
SEQ ID NO: 91 and SEQ ID NO: 90, respectively;
SEQ ID NO: 94 and SEQ ID NO: 93, respectively;
SEQ ID NO: 97 and SEQ ID NO: 96, respectively;
SEQ ID NO: 100 and SEQ ID NO: 99, respectively;
SEQ ID NO: 103 and SEQ ID NO: 102, respectively;
SEQ ID NO: 106 and SEQ ID NO: 105, respectively;
SEQ ID NO: 109 and SEQ ID NO: 108, respectively;
SEQ ID NO: 112 and SEQ ID NO: 111, respectively;
SEQ ID NO: 115 and SEQ ID NO: 114, respectively;
SEQ ID NO: 118 and SEQ ID NO: 117, respectively;
SEQ ID NO: 121 and SEQ ID NO: 120, respectively;
SEQ ID NO: 124 and SEQ ID NO: 123, respectively;
SEQ ID NO: 127 and SEQ ID NO: 126, respectively;
SEQ ID NO: 130 and SEQ ID NO: 129, respectively;
SEQ ID NO: 133 and SEQ ID NO: 132, respectively;
SEQ ID NO: 136 and SEQ ID NO: 135, respectively;
SEQ ID NO: 139 and SEQ ID NO: 138, respectively;
SEQ ID NO: 142 and SEQ ID NO: 141, respectively;
SEQ ID NO: 145 and SEQ ID NO: 144, respectively;
SEQ ID NO: 148 and SEQ ID NO: 147, respectively;
SEQ ID NO: 151 and SEQ ID NO: 150, respectively;
SEQ ID NO: 155 and SEQ ID NO: 154, respectively;
SEQ ID NO: 158 and SEQ ID NO: 157, respectively;
SEQ ID NO: 161 and SEQ ID NO: 160, respectively;
SEQ ID NO: 164 and SEQ ID NO: 163, respectively;
SEQ ID NO: 167 and SEQ ID NO: 166, respectively;
SEQ ID NO: 170 and SEQ ID NO: 169, respectively;
SEQ ID NO: 173 and SEQ ID NO: 172, respectively;
SEQ ID NO: 176 and SEQ ID NO: 175, respectively;
SEQ ID NO: 179 and SEQ ID NO: 178, respectively;
SEQ ID NO: 182 and SEQ ID NO: 181, respectively;
SEQ ID NO: 185 and SEQ ID NO: 184, respectively;
SEQ ID NO: 188 and SEQ ID NO: 187, respectively;
SEQ ID NO: 191 and SEQ ID NO: 190, respectively;
SEQ ID NO: 194 and SEQ ID NO: 193, respectively;
SEQ ID NO: 197 and SEQ ID NO: 196, respectively;
SEQ ID NO: 200 and SEQ ID NO: 199, respectively;
SEQ ID NO: 203 and SEQ ID NO: 202, respectively;
SEQ ID NO: 1100 and SEQ ID NO: 1101, respectively;
SEQ ID NO: 1346 and SEQ ID NO: 1347, respectively;
SEQ ID NO: 1348 and SEQ ID NO: 1349, respectively;
SEQ ID NO: 1350 and SEQ ID NO: 1351, respectively;
SEQ ID NO: 1350 and SEQ ID NO: 1353, respectively;
SEQ ID NO: 1346 and SEQ ID NO: 1349, respectively;
SEQ ID NO: 1356 and SEQ ID NO: 1357, respectively;
SEQ ID NO: 1350 and SEQ ID NO: 1349, respectively;
SEQ ID NO: 1360 and SEQ ID NO: 1361, respectively;
SEQ ID NO: 1362 and SEQ ID NO: 1361, respectively;
SEQ ID NO: 1350 and SEQ ID NO: 1365, respectively;
SEQ ID NO: 1350 and SEQ ID NO: 1367, respectively;
SEQ ID NO: 1350 and SEQ ID NO: 1369, respectively;
SEQ ID NO: 1370 and SEQ ID NO: 1365, respectively;
SEQ ID NO: 1370 and SEQ ID NO: 1367, respectively;
SEQ ID NO: 1370 and SEQ ID NO: 1369, respectively;
SEQ ID NO: 1344 and SEQ ID NO: 1361, respectively;
SEQ ID NO: 15 and SEQ ID NO: 16, respectively;
SEQ ID NO: 23 and SEQ ID NO: 26, respectively;
SEQ ID NO: 24 and SEQ ID NO: 27, respectively;
SEQ ID NO: 25 and SEQ ID NO: 28, respectively;
SEQ ID NO: 232 and SEQ ID NO: 7488, respectively;
SEQ ID NO: 1325 and SEQ ID NO: 1324, respectively;
SEQ ID NO: 3091 and SEQ ID NO: 3092, respectively;
SEQ ID NO: 3183 and SEQ ID NO: 3184, respectively;
SEQ ID NO: 1112 and SEQ ID NO: 1111, respectively;
SEQ ID NO: 1130 and SEQ ID NO: 7490, respectively;
SEQ ID NO: 1152 and SEQ ID NO: 1151, respectively;
SEQ ID NO: 1172 and SEQ ID NO: 7491, respectively;
SEQ ID NO: 1196 and SEQ ID NO: 1195, respectively;
SEQ ID NO: 1219 and SEQ ID NO: 1218, respectively;
SEQ ID NO: 1240 and SEQ ID NO: 1239, respectively;
SEQ ID NO: 1259 and SEQ ID NO: 1258, respectively; or
SEQ ID NO: 1274 and SEQ ID NO: 1278, respectively; or
(c) the second antigen binding domain comprises the sequence of: SEQ ID NO: 80, SEQ ID NO: 83, SEQ ID NO: 86, SEQ ID NO: 89, SEQ ID NO: 92, SEQ ID NO: 95, SEQ ID NO: 98, SEQ ID NO: 101, SEQ ID NO: 104, SEQ ID NO: 107, SEQ ID NO: 110, SEQ ID NO: 113, SEQ ID NO: 116, SEQ ID NO: 119, SEQ ID NO: 122, SEQ ID NO: 125, SEQ ID NO: 128, SEQ ID NO: 131, SEQ ID NO: 134, SEQ ID NO: 137, SEQ ID NO: 140, SEQ ID NO: 143, SEQ ID NO: 146, SEQ ID NO: 149, SEQ ID NO: 153, SEQ ID NO: 156, SEQ ID NO: 159, SEQ ID NO: 162, SEQ ID NO: 165, SEQ ID NO: 168, SEQ ID NO: 171, SEQ ID NO: 174, SEQ ID NO: 177, SEQ ID NO: 180, SEQ ID NO: 183, SEQ ID NO: 186, SEQ ID NO: 189, SEQ ID NO: 192, SEQ ID NO: 195, SEQ ID NO: 198, SEQ ID NO: 201, SEQ ID NO: 204, SEQ ID NO: 206, SEQ ID NO: 208, SEQ ID NO: 210, SEQ ID NO: 212, SEQ ID NO: 214, SEQ ID NO: 216, SEQ ID NO: 218, SEQ ID NO: 220, SEQ ID NO: 222, SEQ ID NO: 224, SEQ ID NO: 1309, SEQ ID NO: 3281, SEQ ID NO: 7492 SEQ ID NO: 1326-1342, or SEQ ID NO: 1376.

145. The composition of claim 136, wherein the second antigen binding domain binds to NKp30 and comprises:
(a) the VHCDR1, VHCDR2, and VHCDR3 of the second antigen binding domain comprising the sequences of:
SEQ ID NO: 6000, SEQ ID NO: 6001, SEQ ID NO: 6002, respectively;
SEQ ID NO: 6007, SEQ ID NO: 6008, SEQ ID NO: 6009, respectively;
SEQ ID NO: 7313, SEQ ID NO: 6001, SEQ ID NO: 6002, respectively;
SEQ ID NO: 7313, SEQ ID NO: 6008, SEQ ID NO: 6009, respectively;
SEQ ID NO: 6007, SEQ ID NO: 6001, SEQ ID NO: 7315, respectively;
SEQ ID NO: 7498, SEQ ID NO: 6001, SEQ ID NO: 7315, respectively; or
SEQ ID NO: 7498, SEQ ID NO: 7437, SEQ ID NO: 7315, respectively; and
(b) the VLCDR1, VLCDR2, and VLCDR3 of the second antigen binding domain comprising the sequences of:
SEQ ID NO: 6063, SEQ ID NO: 6064, SEQ ID NO: 7293, respectively;
SEQ ID NO: 6070, SEQ ID NO: 6071, SEQ ID NO: 6072, respectively;
SEQ ID NO: 6070, SEQ ID NO: 6064, SEQ ID NO: 7321, respectively;
SEQ ID NO: 7326, SEQ ID NO: 7327, SEQ ID NO: 7329, respectively;
SEQ ID NO: 7326, SEQ ID NO: 7327, SEQ ID NO: 7416, respectively;
SEQ ID NO: 7494, SEQ ID NO: 7496, SEQ ID NO: 44, respectively;
SEQ ID NO: 7326, SEQ ID NO: 7327, SEQ ID NO: 44, respectively; or
SEQ ID NO: 7326, SEQ ID NO: 7327, SEQ ID NO: 7447, respectively.

146. The composition of claim 136, wherein the second antigen binding domain binds to NKp30, and wherein the VHCDR1, a VHCDR2, and a VHCDR3 of the second antigen domain comprise the sequences of:
SEQ ID NOs: 6000, 6001, 6002, 6063, 6064, and 7293, respectively;
SEQ ID NO: 6007, 6008, 6009, 6070, 6071, and 6072, respectively;
SEQ ID NOs: 7313, 6001, 6002, 6063, 6064, and 7293, respectively;
SEQ ID NO: 7313, 6008, 6009, 6070, 6071, and 6072, respectively;
SEQ ID NO: 7613, 7385, 7315, 6070, 6064, and 7321, respectively;
SEQ ID NO: 7313, 7318, 6009, 6070, 6064, and 7321, respectively;
SEQ ID NO: 7313, 6008, 6009, 6070, 6064, and 7321, respectively;
SEQ ID NOs: 7313, 6001, 7315, 7326, 7327, and 7329, respectively;
SEQ ID NOs: 7498, 6001, 7315, 7326, 7327, and 7416, respectively;
SEQ ID NOs: 7498, 6001, 7315, 7494, 7496, and 44 respectively;
SEQ ID NOs: 7498, 7437, 7315, 7326, 7327, and 7329, respectively; or
SEQ ID NOs: 7498, 7437, 7315, 7326, 7327, and 7447 respectively.

147. The composition of claim 136, wherein the second antigen binding domain binds to NKp30, and wherein:
(a) (i) the VH of the second antigen binding domain comprises a sequence having at least 75% identity to the sequence of SEQ ID NOs: 6121-6134, SEQ ID NO: 7295, SEQ ID NO: 7297, SEQ ID NO: 6122, SEQ ID NO: 7298, SEQ ID NO: 7300-7304, or SEQ ID NO: 7390; and
(ii) the VL of the second antigen binding domain comprises a sequence having at least 75% identity to any one of sequence selected from the group consisting of: SEQ ID NO: 7294, SEQ ID NOs: 6136-6147, SEQ ID NOs: 7296, SEQ ID NO: 7299, SEQ ID NO: 7305-7309; SEQ ID NO: 7395, SEQ ID NO: 7397, SEQ ID NO: 7399, SEQ ID NO: 7401, SEQ ID NO: 7404, or SEQ ID NO: 7405;
(b) the VH and the VL of the second antigen binding domain comprise sequences having at least 75% identity to the sequences of:
SEQ ID NO: 6121 and SEQ ID NO: 7294, respectively;
SEQ ID NO: 6122 and SEQ ID NO: 6136, respectively;
SEQ ID NO: 6123 and SEQ ID NO: 6137, respectively;
SEQ ID NO: 6124 and SEQ ID NO: 6138, respectively;
SEQ ID NO: 6125 and SEQ ID NO: 6139, respectively;
SEQ ID NO: 6126 and SEQ ID NO: 6140, respectively;
SEQ ID NO: 6127 and SEQ ID NO: 6141, respectively;
SEQ ID NO: 6129 and SEQ ID NO: 6142, respectively;
SEQ ID NO: 6130 and SEQ ID NO: 6143, respectively;
SEQ ID NO: 6131 and SEQ ID NO: 6144, respectively;
SEQ ID NO: 6132 and SEQ ID NO: 6145, respectively;
SEQ ID NO: 6133 and SEQ ID NO: 6146, respectively;
SEQ ID NO: 6134 and SEQ ID NO: 6147, respectively;
SEQ ID NO: 7295 and SEQ ID NO: 7296 respectively;
SEQ ID NO: 7297 and SEQ ID NO: 7296, respectively;
SEQ ID NO: 6122 and SEQ ID NO: 6136, respectively;
SEQ ID NO: 7298 and SEQ ID NO: 7299, respectively;
SEQ ID NO: 7300 and SEQ ID NO: 7305, respectively;
SEQ ID NO: 7301 and SEQ ID NO: 7306, respectively;
SEQ ID NO: 7302 and SEQ ID NO: 7307, respectively;
SEQ ID NO: 7303 and SEQ ID NO: 7308, respectively;
SEQ ID NO: 7304 and SEQ ID NO: 7309, respectively;
SEQ ID NO: 7302 and SEQ ID NO: 7395, respectively;
SEQ ID NO: 7302 and SEQ ID NO: 7397, respectively;
SEQ ID NO: 7302 and SEQ ID NO: 7399, respectively;
SEQ ID NO: 7302 and SEQ ID NO: 7401, respectively;
SEQ ID NO: 7390 and SEQ ID NO: 7309, respectively;
SEQ ID NO: 7390 and SEQ ID NO: 7305, respectively;
SEQ ID NO: 7390 and SEQ ID NO: 7404, respectively; or
SEQ ID NO: 7390 and SEQ ID NO: 7405, respectively; or
(c) the second antigen binding domain comprises a sequence having at least 75% identity to the sequence of: SEQ ID NOs: 6187-6190, SEQ ID NO: 7310, SEQ ID NO: 7311, SEQ ID NO: 7406, SEQ ID NO: 7407, SEQ ID NO: 7408, SEQ ID NO: 7409, SEQ ID NO: 7411, SEQ ID NO: 7412, SEQ ID NO: 7413, or SEQ ID NO: 7414.

148. The composition of claim 136, wherein the second antigen binding domain binds to NKp30, and wherein:
(a) (i) the VH of the second antigen binding domain comprises the sequence of SEQ ID NOs: 6121-6134, SEQ ID NO: 7295, SEQ ID NO: 7297, SEQ ID NO: 6122, SEQ ID NO: 7298, SEQ ID NO: 7300-7304, or SEQ ID NO: 7390; and
(ii) the VL of the second antigen binding domain comprises the sequence of: SEQ ID NO: 7294, SEQ ID NOs: 6136-6147, SEQ ID NOs: 7296, SEQ ID NO: 7299, SEQ ID NO: 7305-7309; SEQ ID NO: 7395, SEQ ID NO: 7397, SEQ ID NO: 7399, SEQ ID NO: 7401, SEQ ID NO: 7404, or SEQ ID NO: 7405; or
(b) the VH and the VL of the second antigen binding domain comprise the sequences of:
SEQ ID NO: 6121 and SEQ ID NO: 7294, respectively;
SEQ ID NO: 6122 and SEQ ID NO: 6136, respectively;
SEQ ID NO: 6123 and SEQ ID NO: 6137, respectively;
SEQ ID NO: 6124 and SEQ ID NO: 6138, respectively;
SEQ ID NO: 6125 and SEQ ID NO: 6139, respectively;
SEQ ID NO: 6126 and SEQ ID NO: 6140, respectively;
SEQ ID NO: 6127 and SEQ ID NO: 6141, respectively;
SEQ ID NO: 6129 and SEQ ID NO: 6142, respectively;
SEQ ID NO: 6130 and SEQ ID NO: 6143, respectively;
SEQ ID NO: 6131 and SEQ ID NO: 6144, respectively;
SEQ ID NO: 6132 and SEQ ID NO: 6145, respectively;
SEQ ID NO: 6133 and SEQ ID NO: 6146, respectively;
SEQ ID NO: 6134 and SEQ ID NO: 6147, respectively;
SEQ ID NO: 7295 and SEQ ID NO: 7296 respectively;
SEQ ID NO: 7297 and SEQ ID NO: 7296, respectively;
SEQ ID NO: 6122 and SEQ ID NO: 6136, respectively;
SEQ ID NO: 7298 and SEQ ID NO: 7299, respectively;
SEQ ID NO: 7300 and SEQ ID NO: 7305, respectively;
SEQ ID NO: 7301 and SEQ ID NO: 7306, respectively;
SEQ ID NO: 7302 and SEQ ID NO: 7307, respectively;
SEQ ID NO: 7303 and SEQ ID NO: 7308, respectively;
SEQ ID NO: 7304 and SEQ ID NO: 7309, respectively;
SEQ ID NO: 7302 and SEQ ID NO: 7395, respectively;
SEQ ID NO: 7302 and SEQ ID NO: 7397, respectively;
SEQ ID NO: 7302 and SEQ ID NO: 7399, respectively;
SEQ ID NO: 7302 and SEQ ID NO: 7401, respectively;
SEQ ID NO: 7390 and SEQ ID NO: 7309, respectively;
SEQ ID NO: 7390 and SEQ ID NO: 7305, respectively;
SEQ ID NO: 7390 and SEQ ID NO: 7404, respectively; or
SEQ ID NO: 7390 and SEQ ID NO: 7405, respectively; or
(c) the second antigen binding domain comprises the sequence of: SEQ ID NOs: 6187-6190, SEQ ID NO: 7310, SEQ ID NO: 7311, SEQ ID NO: 7406, SEQ ID NO: 7407, SEQ ID NO: 7408, SEQ ID NO: 7409, SEQ ID NO: 7411, SEQ ID NO: 7412, SEQ ID NO: 7413, or SEQ ID NO: 7414.

149. The composition of claim 136, wherein the multifunctional molecule further comprises a tumor-targeting moiety.

150. The composition of claim 149, wherein the tumor-targeting moiety binds to a tumor antigen.

151. The composition of claim 150, wherein the tumor antigen is selected from the group consisting of: G6B, CD34, CD41, P-selectin, Clec2, cKIT, FLT3, MPL, ITGB3, ITGB2, GP5, GP6, GP9, GP1BA, DSC2, FCGR2A, TNFRSF10A, TNFRSF10B, and TM4SF1.

152. The composition of claim 136, wherein the multifunctional molecule further comprises one, two, or all of a cytokine molecule, a cytokine inhibitor molecule, a death receptor signal engager, and a stromal modifying moiety.

153. The composition of claim 152, wherein:
(a) the cytokine molecule is selected from the group consisting of interleukin-2 (IL-2) or functional variant thereof, interleukin-7 (IL-7) or functional variant thereof, interleukin-12 (IL-12) or functional variant thereof, interleukin-15 (IL-15) or functional variant thereof, interleukin-18 (IL-18) or functional variant thereof, interleukin-21 (IL-21) or functional variant thereof, interferon gamma or functional variant thereof, and any combination thereof;
(b) the cytokine inhibitor molecule is a TGF-beta inhibitor;
(c) the death receptor signal engager is selected from the group consisting of a TNF-related apoptosis-inducing ligand (TRAIL) molecule, a death receptor molecule, and an antigen binding domain that specifically binds to a death receptor; or
(d) any combination thereof.

154. The composition of claim 136, wherein the multifunctional molecule comprises a dimerization module wherein the dimerization molecule comprises a first immunoglobulin constant region (Fc region) and the second portion of the dimerization module comprises a second Fc region.

155. The composition of claim 154, wherein the first Fc region, the second Fc region, or both comprise an Fc interface with one or more of: a paired cavity-protuberance, an electrostatic interaction, or a strand-exchange, wherein the dimerization of the first Fc region and the second Fc region is enhanced as indicated by a greater ratio of heteromultimer:homomultimer forms relative to a dimerization of Fc regions with a non-engineered interface.

156. The composition of claim 154, wherein the first Fc region, the second Fc region, or both comprise one or more mutations that result in reduced or ablated affinity for at least one Fc receptor as compared to a corresponding wildtype Fc region.

157. The composition of claim 154, wherein the multifunctional molecule comprises a first polypeptide and a second polypeptide, wherein the first polypeptide and the second polypeptide are non-contiguous,
wherein the first polypeptide comprises each of the following operatively linked together:
(a) the first Fc region; and
(b) the first antigen binding domain; and
wherein the second polypeptide comprises each of the following operatively linked together:
(c) the second Fc region; and
(d) an additional first antigen binding domain.

158. The composition of claim 157, wherein the first antigen binding domain or the additional first antigen binding domain comprise:
(a) a single chain variable fragment (scFv) or a single domain antibody (sdAb); or
(b) a first portion of the first antigen binding domain, wherein the first portion of the first antigen binding domain comprises the VH, wherein when the first polypeptide comprises the first portion of the first antigen binding domain, the multifunctional molecule further comprises a third polypeptide comprising a second portion of the first antigen binding domain, wherein the second portion of the first antigen binding domain comprises the VL, wherein the third polypeptide is non-contiguous with the first polypeptide and the second polypeptide.

159. The composition of claim 157, wherein the multifunctional molecule further comprises a fourth polypeptide, wherein the fourth polypeptide is non-contiguous with the first polypeptide, the second polypeptide, or the third polypeptide when the multifunctional molecule comprises the third polypeptide.

160. The composition of claim 159, wherein the fourth polypeptide comprises:
(a) the second antigen binding domain, wherein the second antigen binding domain comprises a single chain variable fragment (scFv) or a single domain antibody (sdAb); or
(b) a first portion of the second antigen binding domain, wherein the first portion of the second antigen binding domain comprises a heavy chain variable domain (VH), wherein when the fourth polypeptide comprises the first portion of the first antigen binding domain, the multifunctional molecule further comprises a fifth polypeptide comprising a second portion of the first antigen binding domain, wherein the second portion of the second antigen binding domain comprises a light chain variable domain (VL), wherein the fifth polypeptide is non-contiguous with the first polypeptide, the second polypeptide, the third polypeptide when the multifunctional molecule comprises the third polypeptide, or the fourth polypeptide when the multifunctional molecule comprises the fourth polypeptide.

161. The composition of claim 160, wherein the fourth polypeptide is linked to the N-terminus of the first polypeptide, the C-terminus of the first polypeptide, the N-terminus of the second polypeptide, the C-terminus of the second polypeptide, the N-terminus of the third polypeptide when the multifunctional molecule comprises the third polypeptide, the C-terminus of the third polypeptide when the multifunctional molecule comprises the third polypeptide, or any combination thereof.

162. The composition of claim 136, wherein the multifunctional molecule preferentially binds to a myeloproliferative neoplasm cell over a non-tumor cell, wherein the binding between the multifunctional molecule and the myeloproliferative neoplasm cell is more than 10, 20, 30, 40, or 50-fold greater than the binding between the multifunctional molecule and a non-tumor cell.

163. The composition of claim 161, wherein the myeloproliferative neoplasm cell is chosen from a myelofibrosis cell, an essential thrombocythemia cell, a polycythemia vera cell, or a chronic myeloid cancer cell, wherein:
the myeloproliferative neoplasm cell does not comprise a JAK2 V617F mutation, or
the myeloproliferative neoplasm cell does not comprise a MPL mutation.

164. A polynucleotide comprising a sequence encoding the multifunctional molecule of claim 136.

165. A method of making the multifunctional molecule of claim 136 comprising: culturing a cell comprising a polynucleotide that comprises a sequence encoding the multifunctional molecule under conditions suitable for gene expression and/or homo- or heterodimerization.

166. A pharmaceutical composition comprising the multifunctional molecule of claim 136, and a pharmaceutically acceptable carrier, excipient, or stabilizer for use in therapy.

167. A method of treating cancer in a subject in need thereof comprising: administering an effective amount of the multifunctional molecule of claim 136 to the subject, thereby treating the cancer in the subject.
